SEQUENCE LISTING (161 sequences)

SEQ ID NO: 1 - DNA, 1713 bp, Saccharomyces cerevisiae
atgtcacttctaatagattctgtaccaacagttgcttataaggaccaaaaaccgggtacttcaggtttacgtaagaagaccaaggttttcatggatgagcctcattatactgagaacttcattcaagcaacaatgcaatctatccctaatggctcagagggaaccactttagttgttggaggagatggtcgtttctacaacgatgttatcatgaacaagattgccgcagtaggtgctgcaaacggtgtcagaaagttagtcattggtcaaggcggtttactttcaacaccagctgcttctcatataattagaacatacgaggaaaagtgtaccggtggtggtatcatattaactgcctcacacaacccaggcggtccagagaatgatttaggtatcaagtataatttacctaatggtgggccagctccagagagtgtcactaacgctatctgggaagcgtctaaaaaattaactcactataaaattataaagaacttccccaagttgaatttgaacaagcttggtaaaaaccaaaaatatggcccattgttagtggacataattgatcctgccaaagcatacgttcaatttctgaaggaaatttttgattttgacttaattaaaagcttcttagcgaaacagcgcaaagacaaagggtggaagttgttgtttgactccttaaatggtattacaggaccatatggtaaggctatatttgttgatgaatttggtttaccggcagaggaagttcttcaaaattggcaccctttacctgatttcggcggtttacatcccgatccgaatctaacctatgcacgaactcttgttgacagggttgaccgcgaaaaaattgcctttggagcagcctccgatggtgatggtgataggaatatgatttacggttatggccctgctttcgtttcgccaggtgattctgttgccattattgccgaatatgcacccgaaattccatacttcgccaaacaaggtatttatggcttggcacgttcatttcctacatcctcagccattgatcgtgttgcagcaaaaaagggattaagatgttacgaagttccaaccggctggaaattcttctgtgccttatttgatgctaaaaagctatcaatctgtggtgaagaatccttcggtacaggttccaatcatatcagagaaaaggacggtctatgggccattattgcttggttaaatatcttggctatctaccataggcgtaaccctgaaaaggaagcttcgatcaaaactattcaggacgaattttggaacgagtatggccgtactttcttcacaagatacgattacgaacatatcgaatgcgagcaggccgaaaaagttgtagctcttttgagtgaatttgtatcaaggccaaacgtttgtggctcccacttcccagctgatgagtctttaaccgttatcgattgtggtgatttttcgtatagagatctagatggctccatctctgaaaatcaaggccttttcgtaaagttttcgaatgggactaaatttgttttgaggttatccggcacaggcagttctggtgcaacaataagattatacgtagaaaagtatactgataaaaaggagaactatggccaaacagctgacgtcttcttgaaacccgtcatcaactccattgtaaaattcttaagatttaaagaaattttaggaacagacgaaccaacagtccgcacatag

SEQ ID NO: 2 - protein, 570 aa, Saccharomyces cerevisiae
MSLLIDSVPTVAYKDQKPGTSGLRKKTKVFMDEPHYTENFIQATMQSIPNGSEGTTLVVGGDGRFYNDVIMNKIAAVGAANGVRKLVIGQGGLLSTPAASHIIRTYEEKCTGGGIILTASHNPGGPENDLGIKYNLPNGGPAPESVTNAIWEASKKLTHYKIIKNFPKLNLNKLGKNQKYGPLLVDIIDPAKAYVQFLKEIFDFDLIKSFLAKQRKDKGWKLLFDSLNGITGPYGKAIFVDEFGLPAEEVLQNWHPLPDFGGLHPDPNLTYARTLVDRVDREKIAFGAASDGDGDRNMIYGYGPAFVSPGDSVAIIAEYAPEIPYFAKQGIYGLARSFPTSSAIDRVAAKKGLRCYEVPTGWKFFCALFDAKKLSICGEESFGTGSNHIREKDGLWAIIAWLNILAIYHRRNPEKEASIKTIQDEFWNEYGRTFFTRYDYEHIECEQAEKVVALLSEFVSRPNVCGSHFPADESLTVIDCGDFSYRDLDGSISENQGLFVKFSNGTKFVLRLSGTGSSGATIRLYVEKYTDKKENYGQTADVFLKPVINSIVKFLRFKEILGTDEPTVRT

SEQ ID NO: 3 - DNA, 1383 bp, Stevia rebaudiana
atggcagagcaacaaaagatcaaaaagtcacctcacgtcttacttattccatttcctctgcaaggacatatcaacccattcatacaatttgggaaaagattgattagtaagggtgtaaagacaacactggtaaccactatccacactttgaattctactctgaaccactcaaatactactactacaagtatagaaattcaagctatatcagacggatgcgatgagggtggctttatgtctgccggtgaatcttacttggaaacattcaagcaagtgggatccaagtctctggccgatctaatcaaaaagttacagagtgaaggcaccacaattgacgccataatctacgattctatgacagagtgggttttagacgttgctatcgaatttggtattgatggaggttcctttttcacacaagcatgtgttgtgaattctctatactaccatgtgcataaagggttaatctctttaccattgggtgaaactgtttcagttccaggttttccagtgttacaacgttgggaaaccccattgatcttacaaaatcatgaacaaatacaatcaccttggtcccagatgttgtttggtcaattcgctaacatcgatcaagcaagatgggtctttactaattcattctataagttagaggaagaggtaattgaatggactaggaagatctggaatttgaaagtcattggtccaacattgccatcaatgtatttggacaaaagacttgatgatgataaagataatggtttcaatttgtacaaggctaatcatcacgaatgtatgaattggctggatgacaaaccaaaggaatcagttgtatatgttgctttcggctctcttgttaaacatggtccagaacaagttgaggagattacaagagcacttatagactctgacgtaaactttttgtgggtcattaagcacaaagaggaggggaaactgccagaaaacctttctgaagtgataaagaccggaaaaggtctaatcgttgcttggtgtaaacaattggatgttttagctcatgaatctgtaggctgttttgtaacacattgcggattcaactctacactagaagccatttccttaggcgtacctgtcgttgcaatgcctcagttctccgatcagacaaccaacgctaaacttttggacgaaatactaggggtgggtgtcagagttaaagcagacgagaatggtatcgtcagaagagggaacctagcttcatgtatcaaaatgatcatggaagaggaaagaggagttatcataaggaaaaacgcagttaagtggaaggatcttgcaaaggttgccgtccatgaaggcggctcttcagataatgatattgttgaatttgtgtccgaactaatcaaagcctaa

SEQ ID NO: 4 - protein, 460 aa, Stevia rebaudiana
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 5 - DNA, 1586 bp, Stevia rebaudiana
atggatgcaatggctacaactgagaagaaaccacacgtcatcttcataccatttccagcacaaagccacattaaagccatgctcaaactagcacaacttctccaccacaaaggactccagataaccttcgtcaacaccgacttcatccacaaccagtttcttgaatcatcgggcccacattgtctagacggtgcaccgggtttccggttcgaaaccattccggatggtgtttctcacagtccggaagcgagcatcccaatcagagaatcactcttgagatccattgaaaccaacttcttggatcgtttcattgatcttgtaaccaaacttccggatcctccgacttgtattatctcagatgggttcttgtcggttttcacaattgacgctgcaaaaaagcttggaattccggtcatgatgtattggacacttgctgcctgtgggttcatgggtttttaccatattcattctctcattgagaaaggatttgcaccacttaaagatgcaagttacttgacaaatgggtatttggacaccgtcattgattgggttccgggaatggaaggcatccgtctcaaggatttcccgctggactggagcactgacctcaatgacaaagttttgatgttcactacggaagctcctcaaaggtcacacaaggtttcacatcatattttccacacgttcgatgagttggagcctagtattataaaaactttgtcattgaggtataatcacatttacaccatcggcccactgcaattacttcttgatcaaatacccgaagagaaaaagcaaactggaattacgagtctccatggatacagtttagtaaaagaagaaccagagtgtttccagtggcttcagtctaaagaaccaaattccgtcgtttatgtaaattttggaagtactacagtaatgtctttagaagacatgacggaatttggttggggacttgctaatagcaaccattatttcctttggatcatccgatcaaacttggtgataggggaaaatgcagttttgccccctgaacttgaggaacatataaagaaaagaggctttattgctagctggtgttcacaagaaaaggtcttgaagcacccttcggttggagggttcttgactcattgtgggtggggatcgaccatcgagagcttgtctgctggggtgccaatgatatgctggccttattcgtgggaccagctgaccaactgtaggtatatatgcaaagaatgggaggttgggctcgagatgggaaccaaagtgaaacgagatgaagtcaagaggcttgtacaagagttgatgggagaaggaggtcacaaaatgaggaacaaggctaaagattggaaagaaaaggctcgcattgcaatagctcctaacggttcatcttctttgaacatagacaaaatggtcaaggaaatcaccgtgctagcaagaaactagttacaaagttgtttcacattgtgctttctatttaagatgtaactttgttctaatttaatattgtctagatgtattgaaccataagtttagttggtctcaggaattgatttttaatgaaataatggtcattaggggtgagt

SEQ ID NO: 6 - DNA, 1446 bp, Artificial Sequence
  Codon-optimized UGT85C2
atggatgcaatggcaactactgagaaaaagcctcatgtgatcttcattccatttcctgcacaatctcacataaaggcaatgctaaagttagcacaactattacaccataagggattacagataactttcgtgaataccgacttcatccataatcaatttctggaatctagtggccctcattgtttggacggagccccagggtttagattcgaaacaattcctgacggtgtttcacattccccagaggcctccatcccaataagagagagtttactgaggtcaatagaaaccaactttttggatcgtttcattgacttggtcacaaaacttccagacccaccaacttgcataatctctgatggctttctgtcagtgtttactatcgacgctgccaaaaagttgggtatcccagttatgatgtactggactcttgctgcatgcggtttcatgggtttctatcacatccattctcttatcgaaaagggttttgctccactgaaagatgcatcatacttaaccaacggctacctggatactgttattgactgggtaccaggtatggaaggtataagacttaaagattttcctttggattggtctacagaccttaatgataaagtattgatgtttactacagaagctccacaaagatctcataaggtttcacatcatatctttcacacctttgatgaattggaaccatcaatcatcaaaaccttgtctctaagatacaatcatatctacactattggtccattacaattacttctagatcaaattcctgaagagaaaaagcaaactggtattacatccttacacggctactctttagtgaaagaggaaccagaatgttttcaatggctacaaagtaaagagcctaattctgtggtctacgtcaacttcggaagtacaacagtcatgtccttggaagatatgactgaatttggttggggccttgctaattcaaatcattactttctatggattatcaggtccaatttggtaataggggaaaacgccgtattacctccagaattggaggaacacatcaaaaagagaggtttcattgcttcctggtgttctcaggaaaaggtattgaaacatccttctgttggtggtttccttactcattgcggttggggctctacaatcgaatcactaagtgcaggagttccaatgatttgttggccatattcatgggaccaacttacaaattgtaggtatatctgtaaagagtgggaagttggattagaaatgggaacaaaggttaaacgtgatgaagtgaaaagattggttcaggagttgatgggggaaggtggccacaagatgagaaacaaggccaaagattggaaggaaaaagccagaattgctattgctcctaacgggtcatcctctctaaacattgataagatggtcaaagagattacagtcttagccagaaactaa

SEQ ID NO: 7 - protein, 481 aa, Stevia rebaudiana
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 8 - DNA, 1377 bp, Artificial Sequence
  Codon-optimized UGT76G1
atggaaaacaagaccgaaacaacagttagacgtaggcgtagaatcattctgtttccagtaccttttcaagggcacatcaatccaatactacaactagccaacgttttgtactctaaaggtttttctattacaatctttcacaccaatttcaacaaaccaaaaacatccaattacccacatttcacattcagattcatacttgataatgatccacaagatgaacgtatttcaaacttacctacccacggtcctttagctggaatgagaattccaatcatcaatgaacatggtgccgatgagcttagaagagaattagagttacttatgttggcatccgaagaggacgaggaagtctcttgtctgattactgacgctctatggtactttgcccaatctgtggctgatagtttgaatttgaggagattggtactaatgacatccagtctgtttaactttcacgctcatgttagtttaccacaatttgacgaattgggatacttggaccctgatgacaagactaggttagaggaacaggcctctggttttcctatgttgaaagtcaaagatatcaagtctgcctattctaattggcaaatcttgaaagagatcttaggaaagatgatcaaacagacaaaggcttcatctggagtgatttggaacagtttcaaagagttagaagagtctgaattggagactgtaatcagagaaattccagcaccttcattcctgataccattaccaaaacatttgactgcttcctcttcctctttgttggatcatgacagaacagtttttcaatggttggaccaacaaccacctagttctgttttgtacgtgtcatttggtagtacttctgaagtcgatgaaaaggacttccttgaaatcgcaagaggcttagtcgatagtaagcagtcattcctttgggtcgtgcgtccaggtttcgtgaaaggctcaacatgggtcgaaccacttccagatggttttctaggcgaaagaggtagaatagtcaaatgggttcctcaacaggaagttttagctcatggcgctattggggcattctggactcattccggatggaattcaactttagaatcagtatgcgaaggggtacctatgatcttttcagattttggtcttgatcaaccactgaacgcaagatacatgtctgatgttttgaaagtgggtgtatatctagaaaatggctgggaaaggggtgaaatagctaatgcaataagacgtgttatggttgatgaagagggggagtatatcagacaaaacgcaagagtgctgaagcaaaaggccgacgtttctctaatgaagggaggctcttcatacgaatccttagaatctcttgtttcctacatttcatcactgtaa

SEQ ID NO: 9 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 10 - DNA, 1422 bp, Artificial Sequence
  Codon-optimized UGT91D2e
atggctacatctgattctattgttgatgacaggaagcagttgcatgtggctactttcccttggcttgctttcggtcatatactgccttacctacaactatcaaaactgatagctgaaaaaggacataaagtgtcattcctttcaacaactagaaacattcaaagattatcttcccacatatcaccattgattaacgtcgttcaattgacacttccaagagtacaggaattaccagaagatgctgaagctacaacagatgtgcatcctgaagatatcccttacttgaaaaaggcatccgatggattacagcctgaggtcactagattccttgagcaacacagtccagattggatcatatacgactacactcactattggttgccttcaattgcagcatcactaggcatttctagggcacatttcagtgtaaccacaccttgggccattgcttacatgggtccatccgctgatgctatgattaacggcagtgatggtagaactaccgttgaagatttgacaaccccaccaaagtggtttccatttccaactaaagtctgttggagaaaacacgacttagcaagactggttccatacaaggcaccaggaatctcagacggctatagaatgggtttagtccttaaagggtctgactgcctattgtctaagtgttaccatgagtttgggacacaatggctaccacttttggaaacattacaccaagttcctgtcgtaccagttggtctattacctccagaaatccctggtgatgagaaggacgagacttgggtttcaatcaaaaagtggttagacgggaagcaaaaaggctcagtggtatatgtggcactgggttccgaagttttagtatctcaaacagaagttgtggaacttgccttaggtttggaactatctggattgccatttgtctgggcctacagaaaaccaaaaggccctgcaaagtccgattcagttgaattgccagacggctttgtcgagagaactagagatagagggttggtatggacttcatgggctccacaattgagaatcctgagtcacgaatctgtgtgcggtttcctaacacattgtggttctggttctatagttgaaggactgatgtttggtcatccacttatcatgttgccaatctttggtgaccagcctttgaatgcacgtctgttagaagataaacaagttggaattgaaatcccacgtaatgaggaagatggatgtttaaccaaggagtctgtggccagatcattacgttccgttgtcgttgaaaaggaaggcgaaatctacaaggccaatgcccgtgaactttcaaagatctacaatgacacaaaagtagagaaggaatatgtttctcaatttgtagattacctagagaaaaacgctagagccgtagctattgatcatgaatcctaa

SEQ ID NO: 11 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 12 - DNA, 1422 bp, Artificial Sequence
  Codon-optimized UGT91D2e-b
atggctacttctgattccatcgttgacgatagaaagcaattgcatgttgctacttttccatggttggctttcggtcatattttgccatacttgcaattgtccaagttgattgctgaaaagggtcacaaggtttcattcttgtctaccaccagaaacatccaaagattgtcctctcatatctccccattgatcaacgttgttcaattgactttgccaagagtccaagaattgccagaagatgctgaagctactactgatgttcatccagaagatatcccttacttgaaaaaggcttccgatggtttacaaccagaagttactagattcttggaacaacattccccagattggatcatctacgattatactcattactggttgccatccattgctgcttcattgggtatttctagagcccatttctctgttactactccatgggctattgcttatatgggtccatctgctgatgctatgattaacggttctgatggtagaactaccgttgaagatttgactactccaccaaagtggtttccatttccaacaaaagtctgttggagaaaacacgatttggctagattggttccatacaaagctccaggtatttctgatggttacagaatgggtatggttttgaaaggttccgattgcttgttgtctaagtgctatcatgaattcggtactcaatggttgcctttgttggaaacattgcatcaagttccagttgttccagtaggtttgttgccaccagaaattccaggtgacgaaaaagacgaaacttgggtttccatcaaaaagtggttggatggtaagcaaaagggttctgttgtttatgttgctttgggttccgaagctttggtttctcaaaccgaagttgttgaattggctttgggtttggaattgtctggtttgccatttgtttgggcttacagaaaacctaaaggtccagctaagtctgattctgttgaattgccagatggtttcgttgaaagaactagagatagaggtttggtttggacttcttgggctccacaattgagaattttgtctcatgaatccgtctgtggtttcttgactcattgtggttctggttctatcgttgaaggtttgatgtttggtcacccattgattatgttgccaatctttggtgaccaaccattgaacgctagattattggaagataagcaagtcggtatcgaaatcccaagaaatgaagaagatggttgcttgaccaaagaatctgttgctagatctttgagatccgttgtcgttgaaaaagaaggtgaaatctacaaggctaacgctagagaattgtccaagatctacaacgataccaaggtcgaaaaagaatacgtttcccaattcgttgactacttggaaaagaatgctagagctgttgccattgatcatgaatcttga

SEQ ID NO: 13 - protein, 473 aa, Artificial Sequence
  UGT91D2e-b
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGMVLKGSDCLLSKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 14 - DNA, 1389 bp, Oryza sativa
atggactccggctactcctcctcctacgccgccgccgccgggatgcacgtcgtgatctgcccgtggctcgccttcggccacctgctcccgtgcctcgacctcgcccagcgcctcgcgtcgcggggccaccgcgtgtcgttcgtctccacgccgcggaacatatcccgcctcccgccggtgcgccccgcgctcgcgccgctcgtcgccttcgtggcgctgccgctcccgcgcgtcgaggggctccccgacggcgccgagtccaccaacgacgtcccccacgacaggccggacatggtcgagctccaccggagggccttcgacgggctcgccgcgcccttctcggagttcttgggcaccgcgtgcgccgactgggtcatcgtcgacgtcttccaccactgggccgcagccgccgctctcgagcacaaggtgccatgtgcaatgatgttgttgggctctgcacatatgatcgcttccatagcagacagacggctcgagcgcgcggagacagagtcgcctgcggctgccgggcagggacgcccagcggcggcgccaacgttcgaggtggcgaggatgaagttgatacgaaccaaaggctcatcgggaatgtccctcgccgagcgcttctccttgacgctctcgaggagcagcctcgtcgtcgggcggagctgcgtggagttcgagccggagaccgtcccgctcctgtcgacgctccgcggtaagcctattaccttccttggccttatgccgccgttgcatgaaggccgccgcgaggacggcgaggatgccaccgtccgctggctcgacgcgcagccggccaagtccgtcgtgtacgtcgcgctaggcagcgaggtgccactgggagtggagaaggtccacgagctcgcgctcgggctggagctcgccgggacgcgcttcctctgggctcttaggaagcccactggcgtctccgacgccgacctcctccccgccggcttcgaggagcgcacgcgcggccgcggcgtcgtggcgacgagatgggttcctcagatgagcatactggcgcacgccgccgtgggcgcgttcctgacccactgcggctggaactcgaccatcgaggggctcatgttcggccacccgcttatcatgctgccgatcttcggcgaccagggaccgaacgcgcggctaatcgaggcgaagaacgccggattgcaggtggcaagaaacgacggcgatggatcgttcgaccgagaaggcgtcgcggcggcgattcgtgcagtcgcggtggaggaagaaagcagcaaagtgtttcaagccaaagccaagaagctgcaggagatcgtcgcggacatggcctgccatgagaggtacatcgacggattcattcagcaattgagatcttacaaggattga

SEQ ID NO: 15 - DNA, 1389 bp, Artificial Sequence
  Codon-optimized EUGT11
atggatagtggctactcctcatcttatgctgctgccgctggtatgcacgttgtgatctgcccttggttggcctttggtcacctgttaccatgtctggatttagcccaaagactggcctcaagaggccatagagtatcatttgtgtctactcctagaaatatctctcgtttaccaccagtcagacctgctctagctcctctagttgcattcgttgctcttccacttccaagagtagaaggattgccagacggcgctgaatctactaatgacgtaccacatgatagacctgacatggtcgaattgcatagaagagcctttgatggattggcagctccattttctgagttcctgggcacagcatgtgcagactgggttatagtcgatgtatttcatcactgggctgctgcagccgcattggaacataaggtgccttgtgctatgatgttgttagggtcagcacacatgatcgcatccatagctgatagaagattggaaagagctgaaacagaatccccagccgcagcaggacaaggtaggccagctgccgccccaacctttgaagtggctagaatgaaattgattcgtactaaaggtagttcagggatgagtcttgctgaaaggttttctctgacattatctagatcatcattagttgtaggtagatcctgcgtcgagttcgaacctgaaacagtacctttactatctactttgagaggcaaacctattactttccttggtctaatgcctccattacatgaaggaaggagagaagatggtgaagatgctactgttaggtggttagatgcccaacctgctaagtctgttgtttacgttgcattgggttctgaggtaccactaggggtggaaaaggtgcatgaattagcattaggacttgagctggccggaacaagattcctttgggctttgagaaaaccaaccggtgtttctgacgccgacttgctaccagctgggttcgaagagagaacaagaggccgtggtgtcgttgctactagatgggtcccacaaatgagtattctagctcatgcagctgtaggggcctttctaacccattgcggttggaactcaacaatagaaggactgatgtttggtcatccacttattatgttaccaatctttggcgatcagggacctaacgcaagattgattgaggcaaagaacgcaggtctgcaggttgcacgtaatgatggtgatggttcctttgatagagaaggcgttgcagctgccatcagagcagtcgccgttgaggaagagtcatctaaagttttccaagctaaggccaaaaaattacaagagattgtggctgacatggcttgtcacgaaagatacatcgatggtttcatccaacaattgagaagttataaagactaa

SEQ ID NO: 16 - protein, 462 aa, Oryza sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 17 - protein, 465 aa, Artificial Sequence
  UGT91D2e-b-EUGT11 chimera 3
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIARNDGDGSFDREGVAAAIRAVAVEEESSKVFQAKAKKLQEIVADMACHERYIDGFIQQLRSYKD

SEQ ID NO: 18 - protein, 470 aa, Artificial Sequence
  UGT91D2e-b-EUGT11 chimera 7
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGMVLKGSDCLLSKCYHEFGTQWLPLLETLHQVPVVPVGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVPRNEEDGCLTKESVARSLRSVVVEKEGEIYKANARELSKIYNDTKVEKEYVSQFVDYLEKNARAVAIDHES

SEQ ID NO: 19 - DNA, 1086 bp, Artificial Sequence
  Codon-optimized GGPPS
atggctttggtaaacccaaccgctcttttctatggtacctctatcagaacaagacctacaaacttactaaatccaactcaaaagctaagaccagtttcatcatcttccttaccttctttctcatcagttagtgcgattcttactgaaaaacatcaatctaatccttctgagaacaacaatttgcaaactcatctagaaactcctttcaactttgatagttatatgttggaaaaagtcaacatggttaacgaggcgcttgatgcatctgtcccactaaaagacccaatcaaaatccatgaatccatgagatactctttattggcaggcggtaagagaatcagaccaatgatgtgtattgcagcctgcgaaatagtcggaggtaatatccttaacgccatgccagccgcatgtgccgtggaaatgattcatactatgtctttggtgcatgacgatcttccatgtatggataatgatgacttcagaagaggtaaacctatttcacacaaggtctacggggaggaaatggcagtattgaccggcgatgctttactaagtttatctttcgaacatatagctactgctacaaagggtgtatcaaaggatagaatcgtcagagctataggggagttggcccgttcagttggctccgaaggtttagtggctggacaagttgtagatatcttgtcagagggtgctgatgttggattagatcacctagaatacattcacatccacaaaacagcaatgttgcttgagtcctcagtagttattggcgctatcatgggaggaggatctgatcagcagatcgaaaagttgagaaaattcgctagatctattggtctactattccaagttgtggatgacattttggatgttacaaaatctaccgaagagttggggaaaacagctggtaaggatttgttgacagataagacaacttacccaaagttgttaggtatagaaaagtccagagaatttgccgaaaaacttaacaaggaagcacaagagcaattaagtggctttgatagacgtaaggcagctcctttgatcgcgttagccaactacaatgcgtaccgtcaaaattga

SEQ ID NO: 20 - protein, 361 aa, Stevia rebaudiana
MALVNPTALFYGTSIRTRPTNLLNPTQKLRPVSSSSLPSFSSVSAILTEKHQSNPSENNNLQTHLETPFNFDSYMLEKVNMVNEALDASVPLKDPIKIHESMRYSLLAGGKRIRPMMCIAACEIVGGNILNAMPAACAVEMIHTMSLVHDDLPCMDNDDFRRGKPISHKVYGEEMAVLTGDALLSLSFEHIATATKGVSKDRIVRAIGELARSVGSEGLVAGQVVDILSEGADVGLDHLEYIHIHKTAMLLESSVVIGAIMGGGSDQQIEKLRKFARSIGLLFQVVDDILDVTKSTEELGKTAGKDLLTDKTTYPKLLGIEKSREFAEKLNKEAQEQLSGFDRRKAAPLIALANYNAYRQN

SEQ ID NO: 21 - DNA, 1029 bp, Artificial Sequence
  Codon-optimized GGPPS
atggctgagcaacaaatatctaacttgctgtctatgtttgatgcttcacatgctagtcagaaattagaaattactgtccaaatgatggacacataccattacagagaaacgcctccagattcctcatcttctgaaggcggttcattgtctagatacgacgagagaagagtctctttgcctctcagtcataatgctgcctctccagatattgtatcacaactatgtttttccactgcaatgtcttcagagttgaatcacagatggaaatctcaaagattaaaggtggccgattctccttacaactatatcctaacattaccatcaaaaggaattagaggtgcctttatcgattccctgaacgtatggttggaggttccagaggatgaaacatcagtcatcaaggaagttattggtatgctccacaactcttcattaatcattgatgacttccaagataattctccacttagaagaggaaagccatctacccatacagtcttcggccctgcccaggctatcaatactgctacttacgttatagttaaagcaatcgaaaagatacaagacatagtgggacacgatgcattggcagatgttacgggtactattacaactattttccaaggtcaggccatggacttgtggtggacagcaaatgcaatcgttccatcaatacaggaatacttacttatggtaaacgataaaaccggtgctctctttagactgagtttggagttgttagctctgaattccgaagccagtatttctgactctgctttagaaagtttatctagtgctgtttccttgctaggtcaatacttccaaatcagagacgactatatgaacttgatcgataacaagtatacagatcagaaaggcttctgcgaagatcttgatgaaggcaagtactcactaacacttattcatgccctccaaactgattcatccgatctactgaccaacatcctttcaatgagaagagtgcaaggaaagttaacggcacaaaagagatgttggttctggaaatga

SEQ ID NO: 22 - protein, 342 aa, Gibberella fujikuroi
MAEQQISNLLSMFDASHASQKLEITVQMMDTYHYRETPPDSSSSEGGSLSRYDERRVSLPLSHNAASPDIVSQLCFSTAMSSELNHRWKSQRLKVADSPYNYILTLPSKGIRGAFIDSLNVWLEVPEDETSVIKEVIGMLHNSSLIIDDFQDNSPLRRGKPSTHTVFGPAQAINTATYVIVKAIEKIQDIVGHDALADVTGTITTIFQGQAMDLWWTANAIVPSIQEYLLMVNDKTGALFRLSLELLALNSEASISDSALESLSSAVSLLGQYFQIRDDYMNLIDNKYTDQKGFCEDLDEGKYSLTLIHALQTDSSDLLTNILSMRRVQGKLTAQKRCWFWK

SEQ ID NO: 23 - DNA, 903 bp, Artificial Sequence
  Codon-optimized GGPPS
atggaaaagactaaggagaaagcagaacgtatcttgctggagccatacagatacttattacaactaccaggaaagcaagtccgttctaaactatcacaagcgttcaatcactggttaaaagttcctgaagataagttacaaatcattattgaagtcacagaaatgctacacaatgcttctttactgatcgatgatatagaggattcttccaaactgagaagaggttttcctgtcgctcattccatatacggggtaccaagtgtaatcaactcagctaattacgtctacttcttgggattggaaaaagtattgacattagatcatccagacgctgtaaagctattcaccagacaacttcttgaattgcatcaaggtcaaggtttggatatctattggagagacacttatacttgcccaacagaagaggagtacaaagcaatggttctacaaaagactggcggtttgttcggacttgccgttggtctgatgcaacttttctctgattacaaggaggacttaaagcctctgttggataccttgggcttgtttttccagattagagatgactacgctaacttacattcaaaggaatattcagaaaacaaatcattctgtgaagatttgactgaagggaagtttagttttccaacaatccacgccatttggtcaagaccagaatctactcaagtgcaaaacattctgcgtcagagaacagagaatattgacatcaaaaagtattgtgttcagtacttggaagatgttggttcttttgcttacacaagacatacacttagagaattagaggcaaaagcatacaagcaaatagaagcctgtggaggcaatccttctctagtggcattggttaaacatttgtccaaaatgttcaccgaggaaaacaagtaa

SEQ ID NO: 24 - protein, 300 aa, Mus musculus
MEKTKEKAERILLEPYRYLLQLPGKQVRSKLSQAFNHWLKVPEDKLQIIIEVTEMLHNASLLIDDIEDSSKLRRGFPVAHSIYGVPSVINSANYVYFLGLEKVLTLDHPDAVKLFTRQLLELHQGQGLDIYWRDTYTCPTEEEYKAMVLQKTGGLFGLAVGLMQLFSDYKEDLKPLLDTLGLFFQIRDDYANLHSKEYSENKSFCEDLTEGKFSFPTIHAIWSRPESTQVQNILRQRTENIDIKKYCVQYLEDVGSFAYTRHTLRELEAKAYKQIEACGGNPSLVALVKHLSKMFTEENK

SEQ ID NO: 25 - DNA, 1020 bp, Artificial Sequence
  Codon-optimized GGPPS
atggcaagattctattttcttaacgcactattgatggttatctcattacaatcaactacagccttcactccagctaaacttgcttatccaacaacaacaacagctctaaatgtcgcctccgccgaaacttctttcagtctagatgaatacttggcctctaagataggacctatagagtctgccttggaagcatcagtcaaatccagaattccacagaccgataagatctgcgaatctatggcctactctttgatggcaggaggcaagagaattagaccagtgttgtgtatcgctgcatgtgagatgttcggtggatcccaagatgtcgctatgcctactgctgtggcattagaaatgatacacacaatgtctttgattcatgatgatttgccatccatggataacgatgacttgagaagaggtaaaccaacaaaccatgtcgttttcggcgaagatgtagctattcttgcaggtgactctttattgtcaacttccttcgagcacgtcgctagagaaacaaaaggagtgtcagcagaaaagatcgtggatgttatcgctagattaggcaaatctgttggtgccgagggccttgctggcggtcaagttatggacttagaatgtgaagctaaaccaggtaccacattagacgacttgaaatggattcatatccataaaaccgctacattgttacaagttgctgtagcttctggtgcagttctaggtggtgcaactcctgaagaggttgctgcatgcgagttgtttgctatgaatataggtcttgcctttcaagttgccgacgatatccttgatgtaaccgcttcatcagaagatttgggtaaaactgcaggcaaagatgaagctactgataagacaacttacccaaagttattaggattagaagagagtaaggcatacgcaagacaactaatcgatgaagccaaggaaagtttggctccttttggagatagagctgcccctttattggccattgcagatttcattattgatagaaagaattga

SEQ ID NO: 26 - protein, 339 aa, Thalassiosira pseudonana
MARFYFLNALLMVISLQSTTAFTPAKLAYPTTTTALNVASAETSFSLDEYLASKIGPIESALEASVKSRIPQTDKICESMAYSLMAGGKRIRPVLCIAACEMFGGSQDVAMPTAVALEMIHTMSLIHDDLPSMDNDDLRRGKPTNHVVFGEDVAILAGDSLLSTSFEHVARETKGVSAEKIVDVIARLGKSVGAEGLAGGQVMDLECEAKPGTTLDDLKWIHIHKTATLLQVAVASGAVLGGATPEEVAACELFAMNIGLAFQVADDILDVTASSEDLGKTAGKDEATDKTTYPKLLGLEESKAYARQLIDEAKESLAPFGDRAAPLLAIADFIIDRKN

SEQ ID NO: 27 - DNA, 1068 bp, Artificial Sequence
  Codon-optimized GGPPS
atgcacttagcaccacgtagagtccctagaggtagaagatcaccacctgacagagttcctgaaagacaaggtgccttgggtagaagacgtggagctggctctactggctgtgcccgtgctgctgctggtgttcaccgtagaagaggaggaggcgaggctgatccatcagctgctgtgcatagaggctggcaagccggtggtggcaccggtttgcctgatgaggtggtgtctaccgcagccgccttagaaatgtttcatgcttttgctttaatccatgatgatatcatggatgatagtgcaactagaagaggctccccaactgttcacagagccctagctgatcgtttaggcgctgctctggacccagatcaggccggtcaactaggagtttctactgctatcttggttggagatctggctttgacatggtccgatgaattgttatacgctccattgactccacatagactggcagcagtactaccattggtaacagctatgagagctgaaaccgttcatggccaatatcttgatataactagtgctagaagacctgggaccgatacttctcttgcattgagaatagccagatataagacagcagcttacacaatggaacgtccactgcacattggtgcagccctggctggggcaagaccagaactattagcagggctttcagcatacgccttgccagctggagaagccttccaattggcagatgacctgctaggcgtcttcggtgatccaagacgtacagggaaacctgacctagatgatcttagaggtggaaagcatactgtcttagtcgccttggcaagagaacatgccactccagaacagagacacacattggatacattattgggtacaccaggtcttgatagacaaggcgcttcaagactaagatgcgtattggtagcaactggtgcaagagccgaagccgaaagacttattacagagagaagagatcaagcattaactgcattgaacgcattaacactgccacctcctttagctgaggcattagcaagattgacattagggtctacagctcatcctgcctaa

SEQ ID NO: 28 - protein, 355 aa, Streptomyces clavuligerus
MHLAPRRVPRGRRSPPDRVPERQGALGRRRGAGSTGCARAAAGVHRRRGGGEADPSAAVHRGWQAGGGTGLPDEVVSTAAALEMFHAFALIHDDIMDDSATRRGSPTVHRALADRLGAALDPDQAGQLGVSTAILVGDLALTWSDELLYAPLTPHRLAAVLPLVTAMRAETVHGQYLDITSARRPGTDTSLALRIARYKTAAYTMERPLHIGAALAGARPELLAGLSAYALPAGEAFQLADDLLGVFGDPRRTGKPDLDDLRGGKHTVLVALAREHATPEQRHTLDTLLGTPGLDRQGASRLRCVLVATGARAEAERLITERRDQALTALNALTLPPPLAEALARLTLGSTAHPA

SEQ ID NO: 29 - DNA, 993 bp, Artificial Sequence
  Codon-optimized GGPPS
atgtcatatttcgataactacttcaatgagatagttaattccgtgaacgacatcattaagtcttacatctctggcgacgtaccaaaactatacgaagcctcctaccatttgtttacatcaggaggaaagagactaagaccattgatccttacaatttcttctgatcttttcggtggacagagagaaagagcatactatgctggcgcagcaatcgaagttttgcacacattcactttggttcacgatgatatcatggatcaagataacattcgtagaggtcttcctactgtacatgtcaagtatggcctacctttggccattttagctggtgacttattgcatgcaaaagcctttcaattgttgactcaggcattgagaggtctaccatctgaaactatcatcaaggcgtttgatatctttacaagatctatcattatcatatcagaaggtcaagctgtcgatatggaattcgaagatagaattgatatcaaggaacaagagtatttggatatgatatctcgtaaaaccgctgccttattctcagcttcttcttccattggggcgttgatagctggagctaatgataacgatgtgagattaatgtccgatttcggtacaaatcttgggatcgcatttcaaattgtagatgatatacttggtttaacagctgatgaaaaagagctaggaaaacctgttttcagtgatatcagagaaggtaaaaagaccatattagtcattaagactttagaattgtgtaaggaagacgagaaaaagattgtgttaaaagcgctaggcaacaagtcagcatcaaaggaagagttgatgagttctgctgacataatcaaaaagtactcattggattacgcctacaacttagctgagaaatactacaaaaacgccatcgattctctaaatcaagtttcaagtaaaagtgatattccagggaaggcattgaaatatcttgctgaattcaccatcagaagacgtaagtaa

SEQ ID NO: 30 - protein, 330 aa, Sulfolobus acidocaldarius
MSYFDNYFNEIVNSVNDIIKSYISGDVPKLYEASYHLFTSGGKRLRPLILTISSDLFGGQRERAYYAGAAIEVLHTFTLVHDDIMDQDNIRRGLPTVHVKYGLPLAILAGDLLHAKAFQLLTQALRGLPSETIIKAFDIFTRSIIIISEGQAVDMEFEDRIDIKEQEYLDMISRKTAALFSASSSIGALIAGANDNDVRLMSDFGTNLGIAFQIVDDILGLTADEKELGKPVFSDIREGKKTILVIKTLELCKEDEKKIVLKALGNKSASKEELMSSADIIKKYSLDYAYNLAEKYYKNAIDSLNQVSSKSDIPGKALKYLAEFTIRRRK

SEQ ID NO: 31 - DNA, 894 bp, Artificial Sequence
  Codon-optimized GGPPS
atggtcgcacaaactttcaacctggatacctacttatcccaaagacaacaacaagttgaagaggccctaagtgctgctcttgtgccagcttatcctgagagaatatacgaagctatgagatactccctcctggcaggtggcaaaagattaagacctatcttatgtttagctgcttgcgaattggcaggtggttctgttgaacaagccatgccaactgcgtgtgcacttgaaatgatccatacaatgtcactaattcatgatgacctgccagccatggataacgatgatttcagaagaggaaagccaactaatcacaaggtgttcggggaagatatagccatcttagcgggtgatgcgcttttagcttacgcttttgaacatattgcttctcaaacaagaggagtaccacctcaattggtgctacaagttattgctagaatcggacacgccgttgctgcaacaggcctcgttggaggccaagtcgtagaccttgaatctgaaggtaaagctatttccttagaaacattggagtatattcactcacataagactggagccttgctggaagcatcagttgtctcaggcggtattctcgcaggggcagatgaagagcttttggccagattgtctcattacgctagagatataggcttggcttttcaaatcgtcgatgatatcctggatgttactgctacatctgaacagttggggaaaaccgctggtaaagaccaggcagccgcaaaggcaacttatccaagtctattgggtttagaagcctctagacagaaagcggaagagttgattcaatctgctaaggaagccttaagaccttacggttcacaagcagagccactcctagcgctggcagacttcatcacacgtcgtcagcattaa

SEQ ID NO: 32 - protein, 297 aa, Synechococcus sp.
MVAQTFNLDTYLSQRQQQVEEALSAALVPAYPERIYEAMRYSLLAGGKRLRPILCLAACELAGGSVEQAMPTACALEMIHTMSLIHDDLPAMDNDDFRRGKPTNHKVFGEDIAILAGDALLAYAFEHIASQTRGVPPQLVLQVIARIGHAVAATGLVGGQVVDLESEGKAISLETLEYIHSHKTGALLEASVVSGGILAGADEELLARLSHYARDIGLAFQIVDDILDVTATSEQLGKTAGKDQAAAKATYPSLLGLEASRQKAEELIQSAKEALRPYGSQAEPLLALADFITRRQH

SEQ ID NO: 33 - DNA, 1680 bp, Artificial Sequence
  Codon-optimized CDPS
atgaaaaccgggtttatctcaccagcaacagtatttcatcacagaatctcaccagcgaccactttcagacatcacttatcacctgctactacaaactctacaggcattgtcgccttaagagacatcaacttcagatgtaaagcagtttctaaagagtactctgatctgttgcagaaagatgaggcttctttcacaaaatgggacgatgacaaggtgaaagatcatcttgataccaacaaaaacttatacccaaatgatgagattaaggaatttgttgaatcagtaaaggctatgttcggtagtatgaatgacggggagataaacgtctctgcatacgatactgcatgggttgctttggttcaagatgtcgatggatcaggtagtcctcagttcccttcttctttagaatggattgccaacaatcaattgtcagatggatcatggggagatcatttgctgttctcagctcacgatagaatcatcaacacattagcatgcgttattgcacttacaagttggaatgttcatccttctaagtgtgaaaaaggtttgaattttctgagagaaaacatttgcaaattagaagatgaaaacgcagaacatatgccaattggttttgaagtaacattcccatcactaattgatatcgcgaaaaagttgaacattgaagtacctgaggatactccagcacttaaagagatctacgcacgtagagatatcaagttaactaagatcccaatggaagttcttcacaaggtacctactactttgttacattctttggaaggaatgcctgatttggagtgggaaaaactgttaaagctacaatgtaaagatggtagtttcttgttttccccatctagtaccgcattcgccctaatgcaaacaaaagatgagaaatgcttacagtatctaacaaatatcgtcactaagttcaacggtggcgtgcctaatgtgtacccagtcgatttgtttgaacatatttgggttgttgatagactgcagagattggggattgccagatacttcaaatcagagataaaagattgtgtagagtatatcaataagtactggaccaaaaatggaatttgttgggctagaaatactcacgttcaagatatcgatgatacagccatgggattcagagtgttgagagcgcacggttatgacgtcactccagatgtttttagacaatttgaaaaagatggtaaattcgtttgctttgcagggcaatcaacacaagccgtgacaggaatgtttaacgtttacagagcctctcaaatgttgttcccaggggagagaattttggaagatgccaaaaagttctcttacaattacttaaaggaaaagcaaagtaccaacgaattgctggataaatggataatcgctaaagatctacctggtgaagttggttatgctctggatatcccatggtatgcttccttaccaagattggaaactcgttattaccttgaacaatacggcggtgaagatgatgtctggataggcaagacattatacagaatgggttacgtgtccaataacacatatctagaaatggcaaagctggattacaataactatgttgcagtccttcaattagaatggtacacaatacaacaatggtacgtcgatattggtatagagaagttcgaatctgacaacatcaagtcagtcctg

SEQ ID NO: 34 - protein, 787 aa, Stevia rebaudiana
MKTGFISPATVFHHRISPATTFRHHLSPATTNSTGIVALRDINFRCKAVSKEYSDLLQKDEASFTKWDDDKVKDHLDTNKNLYPNDEIKEFVESVKAMFGSMNDGEINVSAYDTAWVALVQDVDGSGSPQFPSSLEWIANNQLSDGSWGDHLLFSAHDRIINTLACVIALTSWNVHPSKCEKGLNFLRENICKLEDENAEHMPIGFEVTFPSLIDIAKKLNIEVPEDTPALKEIYARRDIKLTKIPMEVLHKVPTTLLHSLEGMPDLEWEKLLKLQCKDGSFLFSPSSTAFALMQTKDEKCLQYLTNIVTKFNGGVPNVYPVDLFEHIWVVDRLQRLGIARYFKSEIKDCVEYINKYWTKNGICWARNTHVQDIDDTAMGFRVLRAHGYDVTPDVFRQFEKDGKFVCFAGQSTQAVTGMFNVYRASQMLFPGERILEDAKKFSYNYLKEKQSTNELLDKWIIAKDLPGEVGYALDIPWYASLPRLETRYYLEQYGGEDDVWIGKTLYRMGYVSNNTYLEMAKLDYNNYVAVLQLEWYTIQQWYVDIGIEKFESDNIKSVLVSYYLAAASIFEPERSKERIAWAKTTILVDKITSIFDSSQSSKEDITAFIDKFRNKSSSKKHSINGEPWHEVMVALKKTLHGFALDALMTHSQDIHPQLHQAWEMWLTKLQDGVDVTAELMVQMINMTAGRWVSKELLTHPQYQRLSTVTNSVCHDITKLHNFKENSTTVDSKVQELVQLVFSDTPDDLDQDMKQTFLTVMKTFYYKAWCDPNTINDHISKVFEIVI

SEQ ID NO: 35 - DNA, 1584 bp, Artificial Sequence
  Codon-optimized CDPS
atgcctgatgcacacgatgctccacctccacaaataagacagagaacactagtagatgaggctacccaactgctaactgagtccgcagaagatgcatggggtgaagtcagtgtgtcagaatacgaaacagcaaggctagttgcccatgctacatggttaggtggacacgccacaagagtggccttccttctggagagacaacacgaagacgggtcatggggtccaccaggtggatataggttagtccctacattatctgctgttcacgcattattgacatgtcttgcctctcctgctcaggatcatggcgttccacatgatagacttttaagagctgttgacgcaggcttgactgccttgagaagattggggacatctgactccccacctgatactatagcagttgagctggttatcccatctttgctagagggcattcaacacttactggaccctgctcatcctcatagtagaccagccttctctcaacatagaggctctcttgtttgtcctggtggactagatgggagaactctaggagctttgagatcacacgccgcagcaggtacaccagtaccaggaaaagtctggcacgcttccgagactttgggcttgagtaccgaagctgcttctcacttgcaaccagcccaaggtataatcggtggctctgctgctgccacagcaacatggctaaccagggttgcaccatctcaacagtcagattctgccagaagataccttgaggaattacaacacagatactctggcccagttccttccattacccctatcacatacttcgaaagagcatggttattgaacaattttgcagcagccggtgttccttgtgaggctccagctgctttgttggattccttagaagcagcacttacaccacaaggtgctcctgctggagcaggattgcctccagatgctgatgatacagccgctgtgttgcttgcattggcaacacatgggagaggtagaagaccagaagtactgatggattacaggactgacgggtatttccaatgctttattggggaaaggactccatcaatttcaacaaacgctcacgtattggaaacattagggcatcatgtggcccaacatccacaagatagagccagatacggatcagccatggataccgcatcagcttggctgctggcagctcaaaagcaagatggctcttggttagataaatggcatgcctcaccatactacgctactgtttgttgcacacaagccctagccgctcatgcaagtcctgcaactgcaccagctagacagagagctgtcagatgggttttagccacacaaagatccgatggcggttggggtctatggcattcaactgttgaagagactgcttatgccttacagatcttggccccaccttctggtggtggcaatatcccagtccaacaagcacttactagaggcagagcaagattgtgtggagccttgccactgactcctttatggcatgataaggatttgtatactccagtaagagtagtcagagctgccagagctgctgctctgtacactaccagagatctattgttaccaccattgtaa

SEQ ID NO: 36 - protein, 527 aa, Streptomyces clavuligerus
MPDAHDAPPPQIRQRTLVDEATQLLTESAEDAWGEVSVSEYETARLVAHATWLGGHATRVAFLLERQHEDGSWGPPGGYRLVPTLSAVHALLTCLASPAQDHGVPHDRLLRAVDAGLTALRRLGTSDSPPDTIAVELVIPSLLEGIQHLLDPAHPHSRPAFSQHRGSLVCPGGLDGRTLGALRSHAAAGTPVPGKVWHASETLGLSTEAASHLQPAQGIIGGSAAATATWLTRVAPSQQSDSARRYLEELQHRYSGPVPSITPITYFERAWLLNNFAAAGVPCEAPAALLDSLEAALTPQGAPAGAGLPPDADDTAAVLLALATHGRGRRPEVLMDYRTDGYFQCFIGERTPSISTNAHVLETLGHHVAQHPQDRARYGSAMDTASAWLLAAQKQDGSWLDKWHASPYYATVCCTQALAAHASPATAPARQRAVRWVLATQRSDGGWGLWHSTVEETAYALQILAPPSGGGNIPVQQALTRGRARLCGALPLTPLWHDKDLYTPVRVVRAARAAALYTTRDLLLPPL

SEQ ID NO: 37 - DNA, 1551 bp, Artificial Sequence
  Codon-optimized CDPS
atgaacgccctatccgaacacattttgtctgaattgagaagattattgtctgaaatgagtgatggcggatctgttggtccatctgtgtatgatacggcccaggccctaagattccacggtaacgtaacaggtagacaagatgcatatgcttggttgatcgcccagcaacaagcagatggaggttggggctctgccgactttccactctttagacatgctccaacatgggctgcacttctcgcattacaaagagctgatccacttcctggcgcagcagacgcagttcagaccgcaacaagattcttgcaaagacaaccagatccatacgctcatgccgttcctgaggatgcccctattggtgctgaactgatcttgcctcagttttgtggagaggctgcttggttgttgggaggtgtggccttccctagacacccagccctattaccattaagacaggcttgtttagtcaaactgggtgcagtcgccatgttgccttcaggacacccattgctccactcctgggaggcatggggtacttctccaacaacagcctgtccagacgatgatggttctataggtatctcaccagcagctacagccgcctggagagcccaggctgtgaccagaggctcaactcctcaagtgggcagagctgacgcatacttacaaatggcttcaagagcaacgagatcaggcatagaaggagtcttccctaatgtttggcctataaacgtattcgaaccatgctggtcactgtacactctccatcttgccggtctgttcgcccatccagcactggctgaggctgtaagagttatcgttgctcaacttgaagcaagattgggagtgcatggcctcggaccagctttacattttgctgccgacgctgatgatactgcagttgccttatgcgttctgcatttggctggcagagatcctgcagttgacgcattgagacattttgaaattggtgagctctttgttacattcccaggagagagaaatgctagtgtctctacgaacattcacgctcttcatgctttgagattgttaggtaaaccagctgccggagcaagtgcatacgtcgaagcaaatagaaatccacatggtttgtgggacaacgaaaaatggcacgtttcatggctttatccaactgcacacgccgttgcagctctagctcaaggcaagcctcaatggagagatgaaagagcactagccgctctactacaagctcaaagagatgatggtggttggggagctggtagaggatccactttcgaggaaaccgcctacgctcttttcgctttacacgttatggacggatctgaggaagccacaggcagaagaagaatcgctcaagtcgtcgcaagagccttagaatggatgctagctagacatgccgcacatggattaccacaaacaccactctggattggtaaggaattgtactgtcctactagagtcgtaagagtagctgagctagctggcctgtggttagcattaagatggggtagaagagtattagctgaaggtgctggtgctgcaccttaa

SEQ ID NO: 38 - protein, 516 aa, Bradyrhizobium japonicum
MNALSEHILSELRRLLSEMSDGGSVGPSVYDTAQALRFHGNVTGRQDAYAWLIAQQQADGGWGSADFPLFRHAPTWAALLALQRADPLPGAADAVQTATRFLQRQPDPYAHAVPEDAPIGAELILPQFCGEAAWLLGGVAFPRHPALLPLRQACLVKLGAVAMLPSGHPLLHSWEAWGTSPTTACPDDDGSIGISPAATAAWRAQAVTRGSTPQVGRADAYLQMASRATRSGIEGVFPNVWPINVFEPCWSLYTLHLAGLFAHPALAEAVRVIVAQLEARLGVHGLGPALHFAADADDTAVALCVLHLAGRDPAVDALRHFEIGELFVTFPGERNASVSTNIHALHALRLLGKPAAGASAYVEANRNPHGLWDNEKWHVSWLYPTAHAVAALAQGKPQWRDERALAALLQAQRDDGGWGAGRGSTFEETAYALFALHVMDGSEEATGRRRIAQVVARALEWMLARHAAHGLPQTPLWIGKELYCPTRVVRVAELAGLWLALRWGRRVLAEGAGAAP

SEQ ID NO: 39 - DNA, 2490 bp, Artificial Sequence
  Codon-optimized CDPS
atggttttgtcttcttcttgtactacagtaccacacttatcttcattagctgtcgtgcaacttggtccttggagcagtaggattaaaaagaaaaccgatactgttgcagtaccagccgctgcaggaaggtggagaagggccttggctagagcacagcacacatcagaatccgcagctgtcgcaaagggcagcagtttgacccctatagtgagaactgacgctgagtcaaggagaacaagatggccaaccgatgacgatgacgccgaacctttagtggatgagatcagggcaatgcttacttccatgtctgatggtgacatttccgtgagcgcatacgatacagcctgggtcggattggttccaagattagacggcggtgaaggtcctcaatttccagcagctgtgagatggataagaaataaccagttgcctgacggaagttggggcgatgccgcattattctctgcctatgacaggcttatcaatacccttgcctgcgttgtaactttgacaaggtggtccctagaaccagagatgagaggtagaggactatcttttttgggtaggaacatgtggaaattagcaactgaagatgaagagtcaatgcctattggcttcgaattagcatttccatctttgatagagcttgctaagagcctaggtgtccatgacttcccttatgatcaccaggccctacaaggaatctactcttcaagagagatcaaaatgaagaggattccaaaagaagtgatgcataccgttccaacatcaatattgcacagtttggagggtatgcctggcctagattgggctaaactacttaaactacagagcagcgacggaagttttttgttctcaccagctgccactgcatatgctttaatgaataccggagatgacaggtgttttagctacatcgatagaacagtaaagaaattcaacggcggcgtccctaatgtttatccagtggatctatttgaacatatttgggccgttgatagacttgaaagattaggaatctccaggtacttccaaaaggagatcgaacaatgcatggattatgtaaacaggcattggactgaggacggtatttgttgggcaaggaactctgatgtcaaagaggtggacgacacagctatggcctttagacttcttaggttgcacggctacagcgtcagtcctgatgtgtttaaaaacttcgaaaaggacggtgaatttttcgcatttgtcggacagtctaatcaagctgttaccggtatgtacaacttaaacagagcaagccagatatccttcccaggcgaggatgtgcttcatagagctggtgccttctcatatgagttcttgaggagaaaagaagcagagggagctttgagggacaagtggatcatttctaaagatctacctggtgaagttgtgtatactttggattttccatggtacggcaacttacctagagtcgaggccagagactacctagagcaatacggaggtggtgatgacgtttggattggcaagacattgtataggatgccacttgtaaacaatgatgtatatttggaattggcaagaatggatttcaaccactgccaggctttgcatcagttagagtggcaaggactaaaaagatggtatactgaaaataggttgatggactttggtgtcgcccaagaagatgcccttagagcttattttcttgcagccgcatctgtttacgagccttgtagagctgccgagaggcttgcatgggctagagccgcaatactagctaacgccgtgagcacccacttaagaaatagcccatcattcagagaaaggttagagcattctcttaggtgtagacctagtgaagagacagatggctcctggtttaactcctcaagtggctctgatgcagttttagtaaaggctgtcttaagacttactgattcattagccagggaagcacagccaatccatggaggtgacccagaagatattatacacaagttgttaagatctgcttgggccgagtgggttagggaaaaggcagacgctgccgatagcgtgtgcaatggtagttctgcagtagaacaagagggatcaagaatggtccatgataaacagacctgtctattattggctagaatgatcgaaatttctgccggtagggcagctggtgaagcagccagtgaggacggcgatagaagaataattcaattaacaggctccatctgcgacagtcttaagcaaaaaatgctagtttcacaggaccctgaaaaaaatgaagagatgatgtctcacgtggatgacgaattgaagttgaggattagagagttcgttcaatatttgcttagactaggtgaaaaaaagactggatctagcgaaaccaggcaaacatttttaagtatagtgaaatcatgttactatgctgctcattgcccacctcatgtcgttgatagacacattagtagagtgattttcgagccagtaagtgccgcaaagtaaccgcgg

SEQ ID NO: 40 - protein, 827 aa, Zea mays
MVLSSSCTTVPHLSSLAVVQLGPWSSRIKKKTDTVAVPAAAGRWRRALARAQHTSESAAVAKGSSLTPIVRTDAESRRTRWPTDDDDAEPLVDEIRAMLTSMSDGDISVSAYDTAWVGLVPRLDGGEGPQFPAAVRWIRNNQLPDGSWGDAALFSAYDRLINTLACVVTLTRWSLEPEMRGRGLSFLGRNMWKLATEDEESMPIGFELAFPSLIELAKSLGVHDFPYDHQALQGIYSSREIKMKRIPKEVMHTVPTSILHSLEGMPGLDWAKLLKLQSSDGSFLFSPAATAYALMNTGDDRCFSYIDRTVKKFNGGVPNVYPVDLFEHIWAVDRLERLGISRYFQKEIEQCMDYVNRHWTEDGICWARNSDVKEVDDTAMAFRLLRLHGYSVSPDVFKNFEKDGEFFAFVGQSNQAVTGMYNLNRASQISFPGEDVLHRAGAFSYEFLRRKEAEGALRDKWIISKDLPGEVVYTLDFPWYGNLPRVEARDYLEQYGGGDDVWIGKTLYRMPLVNNDVYLELARMDFNHCQALHQLEWQGLKRWYTENRLMDFGVAQEDALRAYFLAAASVYEPCRAAERLAWARAAILANAVSTHLRNSPSFRERLEHSLRCRPSEETDGSWFNSSSGSDAVLVKAVLRLTDSLAREAQPIHGGDPEDIIHKLLRSAWAEWVREKADAADSVCNGSSAVEQEGSRMVHDKQTCLLLARMIEISAGRAAGEAASEDGDRRIIQLTGSICDSLKQKMLVSQDPEKNEEMMSHVDDELKLRIREFVQYLLRLGEKKTGSSETRQTFLSIVKSCYYAAHCPPHVVDRHISRVIFEPVSAAK

SEQ ID NO: 41 - DNA, 2570 bp, Artificial Sequence
  Codon-optimized CDPS
cttcttcactaaatacttagacagagaaaacagagctttttaaagccatgtctcttcagtatcatgttctaaactccattccaagtacaacctttctcagttctactaaaacaacaatatcttcttctttccttaccatctcaggatctcctctcaatgtcgctagagacaaatccagaagcggttccatacattgttcaaagcttcgaactcaagaatacattaattctcaagaggttcaacatgatttgcctctaatacatgagtggcaacagcttcaaggagaagatgctcctcagattagtgttggaagtaatagtaatgcattcaaagaagcagtgaagagtgtgaaaacgatcttgagaaacctaacggacggggaaattacgatatcggcttacgatacagcttgggttgcattgatcgatgccggagataaaactccggcgtttccctccgccgtgaaatggatcgccgagaaccaactttccgatggttcttggggagatgcgtatctcttctcttatcatgatcgtctcatcaatacccttgcatgcgtcgttgctctaagatcatggaatctctttcctcatcaatgcaacaaaggaatcacgtttttccgggaaaatattgggaagctagaagacgaaaatgatgagcatatgccaatcggattcgaagtagcattcccatcgttgcttgagatagctcgaggaataaacattgatgtaccgtacgattctccggtcttaaaagatatatacgccaagaaagagctaaagcttacaaggataccaaaagagataatgcacaagataccaacaacattgttgcatagtttggaggggatgcgtgatttagattgggaaaagctcttgaaacttcaatctcaagacggatctttcctcttctctccttcctctaccgcttttgcattcatgcagacccgagacagtaactgcctcgagtatttgcgaaatgccgtcaaacgtttcaatggaggagttcccaatgtctttcccgtggatcttttcgagcacatatggatagtggatcggttacaacgtttagggatatcgagatactttgaagaagagattaaagagtgtcttgactatgtccacagatattggaccgacaatggcatatgttgggctagatgttcccatgtccaagacatcgatgatacagccatggcatttaggctcttaagacaacatggataccaagtgtccgcagatgtattcaagaactttgagaaagagggagagtttttctgctttgtggggcaatcaaaccaagcagtaaccggtatgttcaacctataccgggcatcacaattggcgtttccaagggaagagatattgaaaaacgccaaagagttttcttataattatctgctagaaaaacgggagagagaggagttgattgataagtggattataatgaaagacttacctggcgagattgggtttgcgttagagattccatggtacgcaagcttgcctcgagtagagacgagattctatattgatcaatatggtggagaaaacgacgtttggattggcaagactctttataggatgccatacgtgaacaataatggatatctggaattagcaaaacaagattacaacaattgccaagctcagcatcagctcgaatgggacatattccaaaagtggtatgaagaaaataggttaagtgagtggggtgtgcgcagaagtgagcttctcgagtgttactacttagcggctgcaactatatttgaatcagaaaggtcacatgagagaatggtttgggctaagtcaagtgtattggttaaagccatttcttcttcttttggggaatcctctgactccagaagaagcttctccgatcagtttcatgaatacattgccaatgctcgacgaagtgatcatcactttaatgacaggaacatgagattggaccgaccaggatcggttcaggccagtcggcttgccggagtgttaatcgggactttgaatcaaatgtcttttgaccttttcatgtctcatggccgtgacgttaacaatctcctctatctatcgtggggagattggatggaaaaatggaaactatatggagatgaaggagaaggagagctcatggtgaagatgataattctaatgaagaacaatgacctaactaacttcttcacccacactcacttcgttcgtctcgcggaaatcatcaatcgaatctgtcttcctcgccaatacttaaaggcaaggagaaacgatgagaaggagaagacaataaagagtatggagaaggagatggggaaaatggttgagttagcattgtcggagagtgacacatttcgtgacgtcagcatcacgtttcttgatgtagcaaaagcattttactactttgctttatgtggcgatcatctccaaactcacatctccaaagtcttgtttcaaaaagtctagtaacctcatcatcatcatcgatccattaacaatcagtggatcgatgtatccatagatgcgtgaataatatttcatgtagagaaggagaacaaattagatcatgtagggttatca

SEQ ID NO: 42 - protein, 802 aa, Arabidopsis thaliana
MSLQYHVLNSIPSTTFLSSTKTTISSSFLTISGSPLNVARDKSRSGSIHCSKLRTQEYINSQEVQHDLPLIHEWQQLQGEDAPQISVGSNSNAFKEAVKSVKTILRNLTDGEITISAYDTAWVALIDAGDKTPAFPSAVKWIAENQLSDGSWGDAYLFSYHDRLINTLACVVALRSWNLFPHQCNKGITFFRENIGKLEDENDEHMPIGFEVAFPSLLEIARGINIDVPYDSPVLKDIYAKKELKLTRIPKEIMHKIPTTLLHSLEGMRDLDWEKLLKLQSQDGSFLFSPSSTAFAFMQTRDSNCLEYLRNAVKRFNGGVPNVFPVDLFEHIWIVDRLQRLGISRYFEEEIKECLDYVHRYWTDNGICWARCSHVQDIDDTAMAFRLLRQHGYQVSADVFKNFEKEGEFFCFVGQSNQAVTGMFNLYRASQLAFPREEILKNAKEFSYNYLLEKREREELIDKWIIMKDLPGEIGFALEIPWYASLPRVETRFYIDQYGGENDVWIGKTLYRMPYVNNNGYLELAKQDYNNCQAQHQLEWDIFQKWYEENRLSEWGVRRSELLECYYLAAATIFESERSHERMVWAKSSVLVKAISSSFGESSDSRRSFSDQFHEYIANARRSDHHFNDRNMRLDRPGSVQASRLAGVLIGTLNQMSFDLFMSHGRDVNNLLYLSWGDWMEKWKLYGDEGEGELMVKMIILMKNNDLTNFFTHTHFVRLAEIINRICLPRQYLKARRNDEKEKTIKSMEKEMGKMVELALSESDTFRDVSITFLDVAKAFYYFALCGDHLQTHISKVLFQKV

SEQ ID NO: 43 - DNA, 2355 bp, Artificial Sequence
  Codon-optimized KS
atgaatttgagtttgtgtatagcatctccactattgaccaaatctaatagaccagctgctttatcagcaattcatacagctagtacatcccatggtggccaaaccaaccctacgaatctgataatcgatacgaccaaggagagaatacaaaaacaattcaaaaatgttgaaatttcagtttcttcttatgatactgcgtgggttgccatggttccatcacctaattctccaaagtctccatgtttcccagaatgtttgaattggctgattaacaaccagttgaatgatggatcttggggtttagtcaatcacacgcacaatcacaaccatccacttttgaaagattctttatcctcaactttggcttgcatcgtggccctaaagagatggaacgtaggtgaggatcagattaacaaggggcttagtttcattgaatctaacttggcttccgcgactgaaaaatctcaaccatctccaataggattcgatatcatctttccaggtctgttagagtacgccaaaaatctagatatcaacttactgtctaagcaaactgatttctcactaatgttacacaagagagaattagaacaaaagagatgtcattcaaacgaaatggatggttacctagcttatatctctgaaggtcttggtaatctttacgattggaatatggtgaaaaagtaccagatgaaaaatggctcagttttcaattccccttctgcaactgcggcagcattcattaaccatcaaaatccaggatgcctgaactatttgaattcactactagacaaattcggcaacgcagttccaactgtataccctcacgatttgtttatcagattgagtatggtggatacaattgaaagacttggtatatcccaccactttagagtcgagatcaaaaatgttttggatgagacataccgttgttgggtggagagagatgaacaaatctttatggatgttgtgacgtgcgcgttggcctttagattgttgcgtattaacggttacgaagttagtccagatccacttgccgaaattacaaacgaattagctttaaaggatgaatacgccgctcttgaaacatatcatgcgtcacatatcctttaccaagaggacttatcatctggaaaacaaattcttaaatctgctgatttcctgaaggaaatcatatccactgatagtaatagactgtccaaactgatccataaagaggttgaaaatgcacttaagttccctattaacaccggcttagaacgtattaacacaagacgtaacatccagctttacaacgtagacaatactagaatcttgaaaaccacttaccattcttccaacatatcaaacactgattacctaagattagctgttgaagatttctacacatgtcagtctatctatagagaagagctgaaaggattagagagatgggtcgttgagaataagctagatcaattgaaatttgccagacaaaagacagcttattgttacttctcagttgccgccactttatcaagtccagaattgtcagatgcacgtatttcttgggctaaaaacggaattttgacaactgttgttgatgatttctttgatattggcgggacaatcgacgaattgacaaacctgattcaatgcgttgaaaagtggaatgtcgatgtcgataaagactgttgctcagaacatgttagaatactgttcttggctctgaaagatgctatctgttggatcggggatgaggctttcaaatggcaagctagagatgtgacgtctcacgtcattcaaacctggctagaactgatgaactctatgttgagagaagcaatttggactagagatgcatacgttcctacattaaacgagtatatggaaaacgcttatgtctcctttgctttgggtcctatcgttaagcctgccatatactttgtaggaccaaagctatccgaggaaatcgtcgaatcatcagaataccataacttgttcaagttaatgtccacacaaggcagattacttaatgatattcattctttcaaaagagagtttaaggaaggaaagttaaatgctgttgctctgcatctttctaatggcgaaagtggtaaagtcgaagaggaagtagttgaggaaatgatgatgatgatcaaaaacaagagaaaggagttgatgaaactaatcttcgaagagaacggttcaattgttcctagagcatgtaaggatgcattttggaacatgtgtcatgtgctaaactttttctacgcaaacgacgatggttttactgggaacacaatactagatacagtaaaagacatcatatacaaccctttggtcttagtaaacgaaaacgaggagcaaagataa

SEQ ID NO: 44 - protein, 784 aa, Stevia rebaudiana
MNLSLCIASPLLTKSNRPAALSAIHTASTSHGGQTNPTNLIIDTTKERIQKQFKNVEISVSSYDTAWVAMVPSPNSPKSPCFPECLNWLINNQLNDGSWGLVNHTHNHNHPLLKDSLSSTLACIVALKRWNVGEDQINKGLSFIESNLASATEKSQPSPIGFDIIFPGLLEYAKNLDINLLSKQTDFSLMLHKRELEQKRCHSNEMDGYLAYISEGLGNLYDWNMVKKYQMKNGSVFNSPSATAAAFINHQNPGCLNYLNSLLDKFGNAVPTVYPHDLFIRLSMVDTIERLGISHHFRVEIKNVLDETYRCWVERDEQIFMDVVTCALAFRLLRINGYEVSPDPLAEITNELALKDEYAALETYHASHILYQEDLSSGKQILKSADFLKEIISTDSNRLSKLIHKEVENALKFPINTGLERINTRRNIQLYNVDNTRILKTTYHSSNISNTDYLRLAVEDFYTCQSIYREELKGLERWVVENKLDQLKFARQKTAYCYFSVAATLSSPELSDARISWAKNGILTTVVDDFFDIGGTIDELTNLIQCVEKWNVDVDKDCCSEHVRILFLALKDAICWIGDEAFKWQARDVTSHVIQTWLELMNSMLREAIWTRDAYVPTLNEYMENAYVSFALGPIVKPAIYFVGPKLSEEIVESSEYHNLFKLMSTQGRLLNDIHSFKREFKEGKLNAVALHLSNGESGKVEEEVVEEMMMMIKNKRKELMKLIFEENGSIVPRACKDAFWNMCHVLNFFYANDDGFTGNTILDTVKDIIYNPLVLVNENEEQR

SEQ ID NO: 45 - DNA, 2355 bp, Artificial Sequence
  Codon-optimized KS
atgaatctgtccctttgtatagctagtccactgttgacaaaatcttctagaccaactgctctttctgcaattcatactgccagtactagtcatggaggtcaaacaaacccaacaaatttgataatcgatactactaaggagagaatccaaaagctattcaaaaatgttgaaatctcagtatcatcttatgacaccgcatgggttgcaatggtgccatcacctaattccccaaaaagtccatgttttccagagtgcttgaattggttaatcaataatcagttaaacgatggttcttggggtttagtcaaccacactcataaccacaatcatccattattgaaggactctttatcatcaacattagcctgtattgttgcattgaaaagatggaatgtaggtgaagatcaaatcaacaagggtttatcattcatagaatccaatctagcttctgctaccgacaaatcacaaccatctccaatcgggttcgacataatcttccctggtttgctggagtatgccaaaaaccttgatatcaacttactgtctaaacaaacagatttctctttgatgctacacaaaagagagttagagcagaaaagatgccattctaacgaaattgacgggtacttagcatatatctcagaaggtttgggtaatttgtatgactggaacatggtcaaaaagtatcagatgaaaaatggatccgtattcaattctccttctgcaactgccgcagcattcattaatcatcaaaaccctgggtgtcttaactacttgaactcactattagataagtttggaaatgcagttccaacagtctatcctttggacttgtacatcagattatctatggttgacactatagagagattaggtatttctcatcatttcagagttgagatcaaaaatgttttggacgagacatacagatgttgggtcgaaagagatgagcaaatctttatggatgtcgtgacctgcgctctggcttttagattgctaaggatacacggatacaaagtatctcctgatcaactggctgagattacaaacgaactggctttcaaagacgaatacgccgcattagaaacataccatgcatcccaaatactttaccaggaagacctaagttcaggaaaacaaatcttgaagtctgcagatttcctgaaaggcattctgtctacagatagtaataggttgtctaaattgatacacaaggaagtagaaaacgcactaaagtttcctattaacactggtttagagagaatcaatactaggagaaacattcagctgtacaacgtagataatacaaggattcttaagaccacctaccatagttcaaacatttccaacacctattacttaagattagctgtcgaagacttttacacttgtcaatcaatctacagagaggagttaaagggcctagaaagatgggtagttcaaaacaagttggatcaactgaagtttgctagacagaagacagcatactgttatttctctgttgctgctaccctttcatccccagaattgtctgatgccagaataagttgggccaaaaatggtattcttacaactgtagtcgatgatttctttgatattggaggtactattgatgaactgacaaatcttattcaatgtgttgaaaagtggaacgtggatgtagataaggattgctgcagtgaacatgtgagaatacttttcctggctctaaaagatgcaatatgttggattggcgacgaggccttcaagtggcaagctagagatgttacatctcatgtcatccaaacttggcttgaactgatgaactcaatgctaagagaagcaatctggacaagagatgcatacgttccaacattgaacgaatacatggaaaacgcttacgtctcatttgccttgggtcctattgttaagccagccatatactttgttgggccaaagttatccgaagagattgttgagtcttccgaatatcataacctattcaagttaatgtcaacacaaggcagacttctgaacgatatccactccttcaaaagagaattcaaggaaggtaagctaaacgctgttgctttgcacttgtctaatggtgaatctggcaaagtggaagaggaagtcgttgaggaaatgatgatgatgatcaaaaacaagagaaaggaattgatgaaattgattttcgaggaaaatggttcaatcgtacctagagcttgtaaagatgctttttggaatatgtgccatgttcttaacttcttttacgctaatgatgatggcttcactggaaatacaatattggatacagttaaagatatcatctacaacccacttgttttggtcaatgagaacgaggaacaaagataa

SEQ ID NO: 46 - protein, 784 aa, Stevia rebaudiana
MNLSLCIASPLLTKSSRPTALSAIHTASTSHGGQTNPTNLIIDTTKERIQKLFKNVEISVSSYDTAWVAMVPSPNSPKSPCFPECLNWLINNQLNDGSWGLVNHTHNHNHPLLKDSLSSTLACIVALKRWNVGEDQINKGLSFIESNLASATDKSQPSPIGFDIIFPGLLEYAKNLDINLLSKQTDFSLMLHKRELEQKRCHSNEIDGYLAYISEGLGNLYDWNMVKKYQMKNGSVFNSPSATAAAFINHQNPGCLNYLNSLLDKFGNAVPTVYPLDLYIRLSMVDTIERLGISHHFRVEIKNVLDETYRCWVERDEQIFMDVVTCALAFRLLRIHGYKVSPDQLAEITNELAFKDEYAALETYHASQILYQEDLSSGKQILKSADFLKGILSTDSNRLSKLIHKEVENALKFPINTGLERINTRRNIQLYNVDNTRILKTTYHSSNISNTYYLRLAVEDFYTCQSIYREELKGLERWVVQNKLDQLKFARQKTAYCYFSVAATLSSPELSDARISWAKNGILTTVVDDFFDIGGTIDELTNLIQCVEKWNVDVDKDCCSEHVRILFLALKDAICWIGDEAFKWQARDVTSHVIQTWLELMNSMLREAIWTRDAYVPTLNEYMENAYVSFALGPIVKPAIYFVGPKLSEEIVESSEYHNLFKLMSTQGRLLNDIHSFKREFKEGKLNAVALHLSNGESGKVEEEVVEEMMMMIKNKRKELMKLIFEENGSIVPRACKDAFWNMCHVLNFFYANDDGFTGNTILDTVKDIIYNPLVLVNENEEQR

SEQ ID NO: 47 - DNA, 1773 bp, Artificial Sequence
  Codon-optimized KS
atggctatgccagtgaagctaacacctgcgtcattatccttaaaagctgtgtgctgcagattctcatccggtggccatgctttgagattcgggagtagtctgccatgttggagaaggacccctacccaaagatctacttcttcctctactactagaccagctgccgaagtgtcatcaggtaagagtaaacaacatgatcaggaagctagtgaagcgactatcagacaacaattacaacttgtggatgtcctggagaatatgggaatatccagacattttgctgcagagataaagtgcatactagacagaacttacagatcttggttacaaagacacgaggaaatcatgctggacactatgacatgtgctatggcttttagaatcctaagattgaacggatacaacgtttcatcagatgaactataccacgttgtagaggcatctggtctgcataattctttgggtgggtatcttaacgataccagaacactacttgaattacacaaggcttcaacagttagtatctctgaggatgaatctatcttagattcaattggctctagatccagaacattgcttagagaacaattggagtctggtggcgcactgagaaagccttctttattcaaagaggttgaacatgcactggatggacctttttacaccacacttgatagacttcatcataggtggaatattgaaaacttcaacattattgagcaacacatgttggagactccatacttatctaaccagcatacatcaagggatatcctagcattgtcaattagagatttttcctcctcacaattcacttatcaacaagagctacagcatctggagagttgggttaaggaatgtagattagatcaactacagttcgcaagacagaaattagcgtacttttacctatcagccgcaggcaccatgttttctcctgagctttctgatgcgagaacattatgggccaaaaacggggtgttgacaactattgttgatgatttctttgatgttgccggttctaaagaggaattggaaaacttagtcatgctggtcgaaatgtgggatgaacatcacaaagttgaattctattctgagcaggtcgaaatcatcttctcttccatctacgattctgtcaaccaattgggtgagaaggcctctttggttcaagacagatcaattacaaaacaccttgttgaaatatggttagacttgttaaagtccatgatgacggaagttgaatggagactgtcaaaatacgtgcctacagaaaaggaatacatgattaatgcctctcttatcttcggcctaggtccaatcgttttaccagctttgtatttcgttggtccaaagatttcagaaagtatagtaaaggacccagaatatgatgaattgttcaaactaatgtcaacatgtggtagattgttgaatgacgtgcaaacgttcgaaagagaatacaatgagggtaaactgaattctgtcagtctattggttcttcacggaggcccaatgtctatttcagacgcaaagaggaaattacaaaagcctattgatacgtgtagaagagatcttctttctttggtccttagagaagagtctgtagtaccaagaccatgtaaggaactattctggaaaatgtgtaaagtgtgctatttcttttactcaacaactgatgggttttctagtcaagtcgaaagagcaaaagaggtagacgctgtcataaatgagccactgaagttgcaaggttctcatacactggtatctgatgtttaa

SEQ ID NO: 48 - protein, 590 aa, Zea mays
MAMPVKLTPASLSLKAVCCRFSSGGHALRFGSSLPCWRRTPTQRSTSSSTTRPAAEVSSGKSKQHDQEASEATIRQQLQLVDVLENMGISRHFAAEIKCILDRTYRSWLQRHEEIMLDTMTCAMAFRILRLNGYNVSSDELYHVVEASGLHNSLGGYLNDTRTLLELHKASTVSISEDESILDSIGSRSRTLLREQLESGGALRKPSLFKEVEHALDGPFYTTLDRLHHRWNIENFNIIEQHMLETPYLSNQHTSRDILALSIRDFSSSQFTYQQELQHLESWVKECRLDQLQFARQKLAYFYLSAAGTMFSPELSDARTLWAKNGVLTTIVDDFFDVAGSKEELENLVMLVEMWDEHHKVEFYSEQVEIIFSSIYDSVNQLGEKASLVQDRSITKHLVEIWLDLLKSMMTEVEWRLSKYVPTEKEYMINASLIFGLGPIVLPALYFVGPKISESIVKDPEYDELFKLMSTCGRLLNDVQTFEREYNEGKLNSVSLLVLHGGPMSISDAKRKLQKPIDTCRRDLLSLVLREESVVPRPCKELFWKMCKVCYFFYSTTDGFSSQVERAKEVDAVINEPLKLQGSHTLVSDV

SEQ ID NO: 49 - DNA, 2232 bp, Artificial Sequence
  Codon-optimized KS
atgcagaacttccatggtacaaaggaaaggatcaaaaagatgtttgacaagattgaattgtccgtttcttcttatgatacagcctgggttgcaatggtcccatcccctgattgcccagaaacaccttgttttccagaatgtactaaatggatcctagaaaatcagttgggtgatggtagttggtcacttcctcatggcaatccacttctagttaaagatgcattatcttccactcttgcttgtattctggctcttaaaagatggggaatcggtgaggaacagattaacaaaggactgagattcatagaactcaactctgctagtgtaaccgataacgaacaacacaaaccaattggatttgacattatctttccaggtatgattgaatacgctatagacttagacctgaatctaccactaaaaccaactgacattaactccatgttgcatcgtagagcccttgaattgacatcaggtggaggcaaaaatctagaaggtagaagagcttacttggcctacgtctctgaaggaatcggtaagctgcaagattgggaaatggctatgaaataccaacgtaaaaacggatctctgttcaatagtccatcaacaactgcagctgcattcatccatatacaagatgctgaatgcctccactatattcgttctcttctccagaaatttggaaacgcagtccctacaatataccctctcgatatctatgccagactttcaatggtagatgccctggaacgtcttggtattgatagacatttcagaaaggagagaaagttcgttctggatgaaacatacagattttggttgcaaggagaagaggagattttctccgataacgcaacctgtgctttggccttcagaatattgagacttaatggttacgatgtctctcttgaagatcacttctctaactctctgggcggttacttaaaggactcaggagcagctttagaactgtacagagccctccaattgtcttacccagacgagtccctcctggaaaagcaaaattctagaacttcttacttcttaaaacaaggtttatccaatgtctccctctgtggtgacagattgcgtaaaaacataattggagaggtgcatgatgctttaaacttttccgaccacgctaacttacaaagattagctattcgtagaaggattaagcattacgctactgacgatacaaggattctaaaaacttcctacagatgctcaacaatcggtaaccaagattttctaaaacttgcagtggaagatttcaatatctgtcaatcaatacaaagagaggaattcaagcatattgaaagatgggtcgttgaaagacgtctagacaagttaaagttcgctagacaaaaagaggcctattgctatttctcagccgcagcaacattgtttgcccctgaattgtctgatgctagaatgtcttgggccaaaaatggtgtattgacaactgtggttgatgatttcttcgatgtcggaggctctgaagaggaattagttaacttgatagaattgatcgagcgttgggatgtgaatggcagtgcagatttttgtagtgaggaagttgagattatctattctgctatccactcaactatctctgaaataggtgataagtcatttggctggcaaggtagagatgtaaagtctcaagttatcaagatctggctggacttattgaaatcaatgttaactgaagctcaatggtcttcaaacaagtctgttcctaccctagatgagtatatgacaaccgcccatgtttcattcgcacttggtccaattgtacttccagccttatacttcgttggcccaaagttgtcagaagaggttgcaggtcatcctgaactactaaacctctacaaagtcacatctacttgtggcagactactgaatgattggagaagttttaagagagaatccgaggaaggtaagctcaacgctattagtttatacatgatccactccggtggtgcttctacagaagaggaaacaatcgaacatttcaaaggtttgattgattctcagagaaggcaactgttacaattggtgttgcaagagaaggatagtatcatacctagaccatgtaaagatctattttggaatatgattaagttattacacactttctacatgaaagatgatggcttcacctcaaatgagatgaggaatgtagttaaggcaatcattaacgaaccaatctcactggatgaattatga

SEQ ID NO: 50 - protein, 775 aa, Populus trichocarpa
MSCIRPWFCPSSISATLTDPASKLVTGEFKTTSLNFHGTKERIKKMFDKIELSVSSYDTAWVAMVPSPDCPETPCFPECTKWILENQLGDGSWSLPHGNPLLVKDALSSTLACILALKRWGIGEEQINKGLRFIELNSASVTDNEQHKPIGFDIIFPGMIEYAKDLDLNLPLKPTDINSMLHRRALELTSGGGKNLEGRRAYLAYVSEGIGKLQDWEMAMKYQRKNGSLFNSPSTTAAAFIHIQDAECLHYIRSLLQKFGNAVPTIYPLDIYARLSMVDALERLGIDRHFRKERKFVLDETYRFWLQGEEEIFSDNATCALAFRILRLNGYDVSLEDHFSNSLGGYLKDSGAALELYRALQLSYPDESLLEKQNSRTSYFLKQGLSNVSLCGDRLRKNIIGEVHDALNFPDHANLQRLAIRRRIKHYATDDTRILKTSYRCSTIGNQDFLKLAVEDFNICQSIQREEFKHIERWVVERRLDKLKFARQKEAYCYFSAAATLFAPELSDARMSWAKNGVLTTVVDDFFDVGGSEEELVNLIELIERWDVNGSADFCSEEVEIIYSAIHSTISEIGDKSFGWQGRDVKSHVIKIWLDLLKSMLTEAQWSSNKSVPTLDEYMTTAHVSFALGPIVLPALYFVGPKLSEEVAGHPELLNLYKVMSTCGRLLNDWRSFKRESEEGKLNAISLYMIHSGGASTEEETIEHFKGLIDSQRRQLLQLVLQEKDSIIPRPCKDLFWNMIKLLHTFYMKDDGFTSNEMRNVVKAIINEPISLDEL

SEQ ID NO: 51 - DNA, 2358 bp, Artificial Sequence
  Codon-optimized KS
atgtctatcaaccttcgctcctccggttgttcgtctccgatctcagctactttggaacgaggattggactcagaagtacagacaagagctaacaatgtgagctttgagcaaacaaaggagaagattaggaagatgttggagaaagtggagctttctgtttcggcctacgatactagttgggtagcaatggttccatcaccgagctcccaaaatgctccacttttcccacagtgtgtgaaatggttattggataatcaacatgaagatggatcttggggacttgataaccatgaccatcaatctcttaagaaggatgtgttatcatctacactggctagtatcctcgcgttaaagaagtggggaattggtgaaagacaaataaacaagggtctccagtttattgagctgaattctgcattagtcactgatgaaaccatacagaaaccaacagggtttgatattatatttcctgggatgattaaatatgctagagatttgaatctgacgattccattgggctcagaagtggtggatgacatgatacgaaaaagagatctggatcttaaatgtgatagtgaaaagttttcaaagggaagagaagcatatctggcctatgttttagaggggacaagaaacctaaaagattgggatttgatagtcaaatatcaaaggaaaaatgggtcactgtttgattctccagccacaacagcagctgcttttactcagtttgggaatgatggttgtctccgttatctctgttctctccttcagaaattcgaggctgcagttccttcagtttatccatttgatcaatatgcacgccttagtataattgtcactcttgaaagcttaggaattgatagagatttcaaaaccgaaatcaaaagcatattggatgaaacctatagatattggcttcgtggggatgaagaaatatgtttggacttggccacttgtgctttggctttccgattattgcttgctcatggctatgatgtgtcttacgatccgctaaaaccatttgcagaagaatctggtttctctgatactttggaaggatatgttaagaatacgttttctgtgttagaattatttaaggctgctcaaagttatccacatgaatcagctttgaagaagcagtgttgttggactaaacaatatctggagatggaattgtccagctgggttaagacctctgttcgagataaatacctcaagaaagaggtcgaggatgctcttgcttttccctcctatgcaagcctagaaagatcagatcacaggagaaaaatactcaatggttctgctgtggaaaacaccagagttacaaaaacctcatatcgtttgcacaatatttgcacctctgatatcctgaagttagctgtggatgacttcaatttctgccagtccatacaccgtgaagaaatggaacgtcttgataggtggattgtggagaatagattgcaggaactgaaatttgccagacagaagctggcttactgttatttctctggggctgcaactttattttctccagaactatctgatgctcgtatatcgtgggccaaaggtggagtacttacaacggttgtagacgacttctttgatgttggagggtccaaagaagaactggaaaacctcatacacttggtcgaaaagtgggatttgaacggtgttcctgagtacagctcagaacatgttgagatcatattctcagttctaagggacaccattctcgaaacaggagacaaagcattcacctatcaaggacgcaatgtgacacaccacattgtgaaaatttggttggatctgctcaagtctatgttgagagaagccgagtggtccagtgacaagtcaacaccaagcttggaggattacatggaaaatgcgtacatatcatttgcattaggaccaattgtcctcccagctacctatctgatcggacctccacttccagagaagacagtcgatagccaccaatataatcagctctacaagctcgtgagcactatgggtcgtcttctaaatgacatacaaggttttaagagagaaagcgcggaagggaagctgaatgcggtttcattgcacatgaaacacgagagagacaatcgcagcaaagaagtgatcatagaatcgatgaaaggtttagcagagagaaagagggaagaattgcataagctagttttggaggagaaaggaagtgtggttccaagggaatgcaaagaagcgttcttgaaaatgagcaaagtgttgaacttattttacaggaaggacgatggattcacatcaaatgatctgatgagtcttgttaaatcagtgatctacgagcctgttagcttacagaaagaatctttaacttga

SEQ ID NO: 52 - protein, 785 aa, Arabidopsis thaliana
MSINLRSSGCSSPISATLERGLDSEVQTRANNVSFEQTKEKIRKMLEKVELSVSAYDTSWVAMVPSPSSQNAPLFPQCVKWLLDNQHEDGSWGLDNHDHQSLKKDVLSSTLASILALKKWGIGERQINKGLQFIELNSALVTDETIQKPTGFDIIFPGMIKYARDLNLTIPLGSEVVDDMIRKRDLDLKCDSEKFSKGREAYLAYVLEGTRNLKDWDLIVKYQRKNGSLFDSPATTAAAFTQFGNDGCLRYLCSLLQKFEAAVPSVYPFDQYARLSIIVTLESLGIDRDFKTEIKSILDETYRYWLRGDEEICLDLATCALAFRLLLAHGYDVSYDPLKPFAEESGFSDTLEGYVKNTFSVLELFKAAQSYPHESALKKQCCWTKQYLEMELSSWVKTSVRDKYLKKEVEDALAFPSYASLERSDHRRKILNGSAVENTRVTKTSYRLHNICTSDILKLAVDDFNFCQSIHREEMERLDRWIVENRLQELKFARQKLAYCYFSGAATLFSPELSDARISWAKGGVLTTVVDDFFDVGGSKEELENLIHLVEKWDLNGVPEYSSEHVEIIFSVLRDTILETGDKAFTYQGRNVTHHIVKIWLDLLKSMLREAEWSSDKSTPSLEDYMENAYISFALGPIVLPATYLIGPPLPEKTVDSHQYNQLYKLVSTMGRLLNDIQGFKRESAEGKLNAVSLHMKHERDNRSKEVIIESMKGLAERKREELHKLVLEEKGSVVPRECKEAFLKMSKVLNLFYRKDDGFTSNDLMSLVKSVIYEPVSLQKESLT

SEQ ID NO: 53 - DNA, 2952 bp, Artificial Sequence
  Codon-optimized CDPS-KS
atggaatttgatgaaccattggttgacgaagcaagatctttagtgcagcgtactttacaagattatgatgacagatacggcttcggtactatgtcatgtgctgcttatgatacagcctgggtgtctttagttacaaaaacagtcgatgggagaaaacaatggcttttcccagagtgttttgaatttctactagaaacacaatctgatgccggaggatgggaaatcgggaattcagcaccaatcgacggtatattgaatacagctgcatccttacttgctctaaaacgtcacgttcaaactgagcaaatcatccaacctcaacatgaccataaggatctagcaggtagagctgaacgtgccgctgcatctttgagagcacaattggctgcattggatgtgtctacaactgaacacgtcggttttgagataattgttcctgcaatgctagacccattagaagccgaagatccatctctagttttcgattttccagctaggaaacctttgatgaagattcatgatgctaagatgagtagattcaggccagaatacttgtatggcaaacaaccaatgaccgccttacattcattagaggctttcataggcaaaatcgacttcgataaggtaagacaccaccgtacccatgggtctatgatgggttctccttcatctaccgcagcctacttaatgcacgcttcacaatgggatggtgactcagaggcttaccttagacacgtgattaaacacgcagcagggcagggaactggtgctgtaccatctgctttcccatcaacacattttgagtcatcttggattcttaccacattgtttagagctggattttcagcttctcatcttgcctgtgatgagttgaacaagttggtcgagatacttgagggctcattcgagaaggaaggtggggcaatcggttacgctccagggtttcaagcagatgttgatgatactgctaaaacaataagtacattagcagtccttggaagagatgctacaccaagacaaatgatcaaggtatttgaagctaatacacattttagaacataccctggtgaaagagatccttctttgacagctaattgtaatgctctatcagccttactacaccaaccagatgcagcaatgtatggatctcaaattcaaaagattaccaaatttgtctgtgactattggtggaagtctgatggtaagattaaagataagtggaacacttgctacttgtacccatctgtcttattagttgaggttttggttgatcttgttagtttattggagcagggtaaattgcctgatgttttggatcaagagcttcaatacagagtcgccatcacattgttccaagcatgtttaaggccattactagaccaagatgccgaaggatcatggaacaagtctatcgaagccacagcctacggcatccttatcctaactgaagctaggagagtttgtttcttcgacagattgtctgagccattgaatgaggcaatccgtagaggtatcgctttcgccgactctatgtctggaactgaagctcagttgaactacatttggatcgaaaaggttagttacgcacctgcattattgactaaatcctatttgttagcagcaagatgggctgctaagtctcctttaggcgcttccgtaggctcttctttgtggactccaccaagagaaggattggataagcatgtcagattattccatcaagctgagttattcagatcccttccagaatgggaattaagagcctccatgattgaagcagctttgttcacaccacttctaagagcacatagactagacgttttccctagacaagatgtaggtgaagacaaatatcttgatgtagttccattcttttggactgccgctaacaacagagatagaacttacgcttccactctattcctttacgatatgtgttttatcgcaatgttaaacttccagttagacgaattcatggaggccacagccggtatcttattcagagatcatatggatgatttgaggcaattgattcatgatcttttggcagagaaaacttccccaaagagttctggtagaagtagtcagggcacaaaagatgctgactcaggtatagaggaagacgtgtcaatgtccgattcagcttcagattcccaggatagaagtccagaatacgacttggttttcagtgcattgagtacctttacaaaacatgtcttgcaacacccatctatacaaagtgcctctgtatgggatagaaaactacttgctagagagatgaaggcttacttacttgctcatatccaacaagcagaagattcaactccattgtctgaattgaaagatgtgcctcaaaagactgatgtaacaagagtttctacatctactactaccttctttaactgggttagaacaacttccgcagaccatatatcctgcccatactccttccactttgtagcatgccatctaggcgcagcattgtcacctaaagggtctaacggtgattgctatccttcagctggtgagaagttcttggcagctgcagtctgcagacatttggccaccatgtgtagaatgtacaacgatcttggatcagctgaacgtgattctgatgaaggtaatttgaactccttggacttccctgaattcgccgattccgcaggaaacggagggatagaaattcagaaggccgctctattaaggttagctgagtttgagagagattcatacttagaggccttccgtcgtttacaagatgaatccaatagagttcacggtccagccggtggtgatgaagccagattgtccagaaggagaatggcaatccttgaattcttcgcccagcaggtagatttgtacggtcaagtatacgtcattagggatatttccgctcgtattcctaaaaacgaggttgagaaaaagagaaaattggatgatgctttcaattga

SEQ ID NO: 54 - protein, 983 aa, Phomopsis amygdali
MEFDEPLVDEARSLVQRTLQDYDDRYGFGTMSCAAYDTAWVSLVTKTVDGRKQWLFPECFEFLLETQSDAGGWEIGNSAPIDGILNTAASLLALKRHVQTEQIIQPQHDHKDLAGRAERAAASLRAQLAALDVSTTEHVGFEIIVPAMLDPLEAEDPSLVFDFPARKPLMKIHDAKMSRFRPEYLYGKQPMTALHSLEAFIGKIDFDKVRHHRTHGSMMGSPSSTAAYLMHASQWDGDSEAYLRHVIKHAAGQGTGAVPSAFPSTHFESSWILTTLFRAGFSASHLACDELNKLVEILEGSFEKEGGAIGYAPGFQADVDDTAKTISTLAVLGRDATPRQMIKVFEANTHFRTYPGERDPSLTANCNALSALLHQPDAAMYGSQIQKITKFVCDYWWKSDGKIKDKWNTCYLYPSVLLVEVLVDLVSLLEQGKLPDVLDQELQYRVAITLFQACLRPLLDQDAEGSWNKSIEATAYGILILTEARRVCFFDRLSEPLNEAIRRGIAFADSMSGTEAQLNYIWIEKVSYAPALLTKSYLLAARWAAKSPLGASVGSSLWTPPREGLDKHVRLFHQAELFRSLPEWELRASMIEAALFTPLLRAHRLDVFPRQDVGEDKYLDVVPFFWTAANNRDRTYASTLFLYDMCFIAMLNFQLDEFMEATAGILFRDHMDDLRQLIHDLLAEKTSPKSSGRSSQGTKDADSGIEEDVSMSDSASDSQDRSPEYDLVFSALSTFTKHVLQHPSIQSASVWDRKLLAREMKAYLLAHIQQAEDSTPLSELKDVPQKTDVTRVSTSTTTFFNWVRTTSADHISCPYSFHFVACHLGAALSPKGSNGDCYPSAGEKFLAAAVCRHLATMCRMYNDLGSAERDSDEGNLNSLDFPEFADSAGNGGIEIQKAALLRLAEFERDSYLEAFRRLQDESNRVHGPAGGDEARLSRRRMAILEFFAQQVDLYGQVYVIRDISARIPKNEVEKKRKLDDAFN

SEQ ID NO: 55 - DNA, 2646 bp, Artificial Sequence
  Codon-optimized CDPS-KS
atggcttctagtacacttatccaaaacagatcatgtggcgtcacatcatctatgtcaagttttcaaatcttcagaggtcaaccactaagatttcctggcactagaaccccagctgcagttcaatgcttgaaaaagaggagatgccttaggccaaccgaatccgtactagaatcatctcctggctctggttcatatagaatagtaactggcccttctggaattaaccctagttctaacgggcacttgcaagagggttccttgactcacaggttaccaataccaatggaaaaatctatcgataacttccaatctactctatatgtgtcagatatttggtctgaaacactacagagaactgaatgtttgctacaagtaactgaaaacgtccagatgaatgagtggattgaggaaattagaatgtactttagaaatatgactttaggtgaaatttccatgtccccttacgacactgcttgggtggctagagttccagcgttggacggttctcatgggcctcaattccacagatctttgcaatggattatcgacaaccaattaccagatggggactggggcgaaccttctcttttcttgggttacgatagagtttgtaatactttagcctgtgtgattgcgttgaaaacatggggtgttggggcacaaaacgttgaaagaggaattcagttcctacaatctaacatatacaagatggaggaagatgacgctaatcatatgccaataggattcgaaatcgtattccctgctatgatggaagatgccaaagcattaggtttggatttgccatacgatgctactattttgcaacagatttcagccgaaagagagaaaaagatgaaaaagatcccaatggcaatggtgtacaaatacccaaccactttacttcactccttagaaggcttgcatagagaagttgattggaataagttgttacaattacaatctgaaaatggtagttttctttattcacctgcttcaaccgcatgcgccttaatgtacactaaggacgttaaatgttttgattacttaaaccagttgttgatcaagttcgaccacgcatgcccaaatgtatatccagtcgatctattcgaaagattatggatggttgacagattgcagagattagggatctccagatactttgaaagagagattagagattgtttacaatacgtctacagatattggaaagattgtggaatcggatgggcttctaactcttccgtacaagatgttgatgatacagccatggcgtttagacttttaaggactcatggtttcgacgtaaaggaagattgctttagacagtttttcaaggacggagaattcttctgcttcgcaggccaatcatctcaagcagttacaggcatgtttaatctttcaagagccagtcaaacattgtttccaggagaatctttattgaaaaaggctagaaccttctctagaaacttcttgagaacaaagcatgagaacaacgaatgtttcgataaatggatcattactaaagatttggctggtgaagtcgagtataacttgaccttcccatggtatgcctctttgcctagattagaacataggacatacttagatcaatatggaatcgatgatatctggataggcaaatctttatacaaaatgcctgctgttaccaacgaagttttcctaaagttggcaaaggcagactttaacatgtgtcaagctctacacaaaaaggaattggaacaagtgataaagtggaacgcgtcctgtcaattcagagatcttgaattcgccagacaaaaatcagtagaatgctattttgctggtgcagccacaatgttcgaaccagaaatggttcaagctagattagtctgggcaagatgttgtgtattgacaactgtcttagacgattactttgaccacgggacacctgttgaggaacttagagtgtttgttcaagctgtcagaacatggaatccagagttgatcaacggtttgccagagcaagctaaaatcttgtttatgggcttatacaaaacagttaacacaattgcagaggaagcattcatggcacagaaaagagacgtccatcatcatttgaaacactattgggacaagttgataacaagtgccctaaaggaggccgaatgggcagagtcaggttacgtcccaacatttgatgaatacatggaagtagctgaaatttctgttgctctagaaccaattgtctgtagtaccttgttctttgcgggtcatagactagatgaggatgttctagatagttacgattaccatctagttatgcatttggtaaacagagtcggtagaatcttgaatgatatacaaggcatgaagagggaggcttcacaaggtaagatctcatcagttcaaatctacatggaggaacatccatctgttccatctgaggccatggcgatcgctcatcttcaagagttagttgataattcaatgcagcaattgacatacgaagttcttaggttcactgcggttccaaaaagttgtaagagaatccacttgaatatggctaaaatcatgcatgccttctacaaggatactgatggattctcatcccttactgcaatgacaggattcgtcaaaaaggttcttttcgaacctgtgcctgagtaa

SEQ ID NO: 56 - protein, 881 aa, Physcomitrella patens
MASSTLIQNRSCGVTSSMSSFQIFRGQPLRFPGTRTPAAVQCLKKRRCLRPTESVLESSPGSGSYRIVTGPSGINPSSNGHLQEGSLTHRLPIPMEKSIDNFQSTLYVSDIWSETLQRTECLLQVTENVQMNEWIEEIRMYFRNMTLGEISMSPYDTAWVARVPALDGSHGPQFHRSLQWIIDNQLPDGDWGEPSLFLGYDRVCNTLACVIALKTWGVGAQNVERGIQFLQSNIYKMEEDDANHMPIGFEIVFPAMMEDAKALGLDLPYDATILQQISAEREKKMKKIPMAMVYKYPTTLLHSLEGLHREVDWNKLLQLQSENGSFLYSPASTACALMYTKDVKCFDYLNQLLIKFDHACPNVYPVDLFERLWMVDRLQRLGISRYFEREIRDCLQYVYRYWKDCGIGWASNSSVQDVDDTAMAFRLLRTHGFDVKEDCFRQFFKDGEFFCFAGQSSQAVTGMFNLSRASQTLFPGESLLKKARTFSRNFLRTKHENNECFDKWIITKDLAGEVEYNLTFPWYASLPRLEHRTYLDQYGIDDIWIGKSLYKMPAVTNEVFLKLAKADFNMCQALHKKELEQVIKWNASCQFRDLEFARQKSVECYFAGAATMFEPEMVQARLVWARCCVLTTVLDDYFDHGTPVEELRVFVQAVRTWNPELINGLPEQAKILFMGLYKTVNTIAEEAFMAQKRDVHHHLKHYWDKLITSALKEAEWAESGYVPTFDEYMEVAEISVALEPIVCSTLFFAGHRLDEDVLDSYDYHLVMHLVNRVGRILNDIQGMKREASQGKISSVQIYMEEHPSVPSEAMAIAHLQELVDNSMQQLTYEVLRFTAVPKSCKRIHLNMAKIMHAFYKDTDGFSSLTAMTGFVKKVLFEPVPE

SEQ ID NO: 57 - DNA, 2859 bp, Artificial Sequence
  Codon-optimized CDPS-KS
atgcctggtaaaattgaaaatggtaccccaaaggacctcaagactggaaatgattttgtttctgctgctaagagtttactagatcgagctttcaaaagtcatcattcctactacggattatgctcaacttcatgtcaagtttatgatacagcttgggttgcaatgattccaaaaacaagagataatgtaaaacagtggttgtttccagaatgtttccattacctcttaaaaacacaagccgcagatggctcatggggttcattgcctacaacacagacagcgggtatcctagatacagcctcagctgtgctggcattattgtgccacgcacaagagcctttacaaatattggatgtatctccagatgaaatggggttgagaatagaacacggtgtcacatccttgaaacgtcaattagcagtttggaatgatgtggaggacaccaaccatattggcgtcgagtttatcataccagccttactttccatgctagaaaaggaattagatgttccatcttttgaatttccatgtaggtccatcttagagagaatgcacggggagaaattaggtcatttcgacctggaacaagtttacggcaagccaagctcattgttgcactcattggaagcatttctcggtaagctagattttgatcgactatcacatcacctataccacggcagtatgatggcatctccatcttcaacggctgcttatcttattggggctacaaaatgggatgacgaagccgaagattacctaagacatgtaatgcgtaatggtgcaggacatgggaatggaggtatttctggtacatttccaactactcatttcgaatgtagctggattatagcaacgttgttaaaggttggctttactttgaagcaaattgacggcgatggcttaagaggtttatcaaccatcttacttgaggcgcttcgtgatgagaatggtgtcataggctttgcccctagaacagcagatgtagatgacacagccaaagctctattggccttgtcattggtaaaccagccagtgtcacctgatatcatgattaaggtctttgagggcaaagaccattttaccacttttggttcagaaagagatccatcattgacttccaacctgcacgtccttttatctttacttaaacaatctaacttgtctcaataccatcctcaaatcctcaaaacaacattattcacttgtagatggtggtggggttccgatcattgtgtcaaagacaaatggaatttgagtcacctatatccaactatgttgttggttgaagccttcactgaagtgctccatctcattgacggtggtgaattgtctagtctgtttgatgaatcctttaagtgtaagattggtcttagcatctttcaagcggtacttagaataatcctcacccaagacaacgacggctcttggagaggatacagagaacagacgtgttacgcaatattggctttagttcaagcgagacatgtatgctttttcactcacatggttgacagactgcaatcatgtgttgatcgaggtttctcatggttgaaatcttgctcttttcattctcaagacctgacttggacctctaaaacagcttatgaagtgggtttcgtagctgaagcatataaactagctgctttacaatctgcttccctggaggttcctgctgccaccattggacattctgtcacgtctgccgttccatcaagtgatcttgaaaaatacatgagattggtgagaaaaactgcgttattctctccactggatgagtggggtctaatggcttctatcatcgaatcttcatttttcgtaccattactgcaggcacaaagagttgaaatataccctagagataatatcaaggtggacgaagataagtacttgtctattatcccattcacatgggtcggatgcaataataggtctagaactttcgcaagtaacagatggctatacgatatgatgtacctttcattactcggctatcaaaccgacgagtacatggaagctgtagctgggccagtgtttggggatgtttccttgttacatcaaacaattgataaggtgattgataatacaatgggtaaccttgcgagagccaatggaacagtacacagtggtaatggacatcagcacgaatctcctaatataggtcaagtcgaggacaccttgactcgtttcacaaattcagtcttgaatcacaaagacgtccttaactctagctcatctgatcaagatactttgagaagagagtttagaacattcatgcacgctcatataacacaaatcgaagataactcacgattcagtaagcaagcctcatccgatgcgttttcctctcctgaacaatcttactttcaatgggtgaactcaactggtggctcacatgtcgcttgcgcctattcatttgccttctctaattgcctcatgtctgcaaatttgttgcagggtaaagacgcatttccaagcggaacgcaaaagtacttaatctcctctgttatgagacatgccacaaacatgtgtagaatgtataacgactttggctctattgccagagacaacgctgagagaaatgttaatagtattcattttcctgagtttactctctgtaacggaacttctcaaaacctagatgaaaggaaggaaagacttctgaaaatcgcaacttacgaacaagggtatttggatagagcactagaggccttggaaagacagagtagagatgatgccggagacagagctggatctaaagatatgagaaagttgaaaatcgttaagttattctgtgatgttacggacttatacgatcagctctacgttatcaaagatttgtcatcctctatgaagtaa

SEQ ID NO: 58 - protein, 952 aa, Gibberella fujikuroi
MPGKIENGTPKDLKTGNDFVSAAKSLLDRAFKSHHSYYGLCSTSCQVYDTAWVAMIPKTRDNVKQWLFPECFHYLLKTQAADGSWGSLPTTQTAGILDTASAVLALLCHAQEPLQILDVSPDEMGLRIEHGVTSLKRQLAVWNDVEDTNHIGVEFIIPALLSMLEKELDVPSFEFPCRSILERMHGEKLGHFDLEQVYGKPSSLLHSLEAFLGKLDFDRLSHHLYHGSMMASPSSTAAYLIGATKWDDEAEDYLRHVMRNGAGHGNGGISGTFPTTHFECSWIIATLLKVGFTLKQIDGDGLRGLSTILLEALRDENGVIGFAPRTADVDDTAKALLALSLVNQPVSPDIMIKVFEGKDHFTTFGSERDPSLTSNLHVLLSLLKQSNLSQYHPQILKTTLFTCRWWWGSDHCVKDKWNLSHLYPTMLLVEAFTEVLHLIDGGELSSLFDESFKCKIGLSIFQAVLRIILTQDNDGSWRGYREQTCYAILALVQARHVCFFTHMVDRLQSCVDRGFSWLKSCSFHSQDLTWTSKTAYEVGFVAEAYKLAALQSASLEVPAATIGHSVTSAVPSSDLEKYMRLVRKTALFSPLDEWGLMASIIESSFFVPLLQAQRVEIYPRDNIKVDEDKYLSIIPFTWVGCNNRSRTFASNRWLYDMMYLSLLGYQTDEYMEAVAGPVFGDVSLLHQTIDKVIDNTMGNLARANGTVHSGNGHQHESPNIGQVEDTLTRFTNSVLNHKDVLNSSSSDQDTLRREFRTFMHAHITQIEDNSRFSKQASSDAFSSPEQSYFQWVNSTGGSHVACAYSFAFSNCLMSANLLQGKDAFPSGTQKYLISSVMRHATNMCRMYNDFGSIARDNAERNVNSIHFPEFTLCNGTSQNLDERKERLLKIATYEQGYLDRALEALERQSRDDAGDRAGSKDMRKLKIVKLFCDVTDLYDQLYVIKDLSSSMK

SEQ ID NO: 59 - DNA, 1542 bp, Artificial Sequence
  Codon-optimized KO
atggatgctgtgacgggtttgttaactgtcccagcaaccgctataactattggtggaactgctgtagcattggcggtagcgctaatcttttggtacctgaaatcctacacatcagctagaagatcccaatcaaatcatcttccaagagtgcctgaagtcccaggtgttccattgttaggaaatctgttacaattgaaggagaaaaagccatacatgacttttacgagatgggcagcgacatatggacctatctatagtatcaaaactggggctacaagtatggttgtggtatcatctaatgagatagccaaggaggcattggtgaccagattccaatccatatctacaaggaacttatctaaagccctgaaagtacttacagcagataagacaatggtcgcaatgtcagattatgatgattatcataaaacagttaagagacacatactgaccgccgtcttgggtcctaatgcacagaaaaagcatagaattcacagagatatcatgatggataacatatctactcaacttcatgaattcgtgaaaaacaacccagaacaggaagaggtagaccttagaaaaatctttcaatctgagttattcggcttagctatgagacaagccttaggaaaggatgttgaaagtttgtacgttgaagacctgaaaatcactatgaatagagacgaaatctttcaagtccttgttgttgatccaatgatgggagcaatcgatgttgattggagagacttctttccatacctaaagtgggtcccaaacaaaaagttcgaaaatactattcaacaaatgtacatcagaagagaagctgttatgaaatctttaatcaaagagcacaaaaagagaatagcgtcaggcgaaaagctaaatagttatatcgattaccttttatctgaagctcaaactttaaccgatcagcaactattgatgtccttgtgggaaccaatcattgaatcttcagatacaacaatggtcacaacagaatgggcaatgtacgaattagctaaaaaccctaaattgcaagataggttgtacagagacattaagtccgtctgtggatctgaaaagataaccgaagagcatctatcacagctgccttacattacagctattttccacgaaacactgagaagacactcaccagttcctatcattcctctaagacatgtacatgaagataccgttctaggcggctaccatgttcctgctggcacagaacttgccgttaacatctacggttgcaacatggacaaaaacgtttgggaaaatccagaggaatggaacccagaaagattcatgaaagagaatgagacaattgattttcaaaagacgatggccttcggtggtggtaagagagtttgtgctggttccttgcaagcccttttaactgcatctattgggattgggagaatggttcaagagttcgaatggaaactgaaggatatgactcaagaggaagtgaacacgataggcctaactacacaaatgttaagaccattgagagctattatcaaacctaggatctaa

SEQ ID NO: 60 - protein, 513 aa, Stevia rebaudiana
MDAVTGLLTVPATAITIGGTAVALAVALIFWYLKSYTSARRSQSNHLPRVPEVPGVPLLGNLLQLKEKKPYMTFTRWAATYGPIYSIKTGATSMVVVSSNEIAKEALVTRFQSISTRNLSKALKVLTADKTMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEIFQVLVVDPMMGAIDVDWRDFFPYLKWVPNKKFENTIQQMYIRREAVMKSLIKEHKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRRHSPVPIIPLRHVHEDTVLGGYHVPAGTELAVNIYGCNMDKNVWENPEEWNPERFMKENETIDFQKTMAFGGGKRVCAGSLQALLTASIGIGRMVQEFEWKLKDMTQEEVNTIGLTTQMLRPLRAIIKPRI

SEQ ID NO: 61 - DNA, 1566 bp, Artificial Sequence
  Codon-optimized KO
aagcttactagtaaaatggacggtgtcatcgatatgcaaaccattccattgagaaccgctattgctattggtggtactgctgttgctttggttgttgcattatacttttggttcttgagatcctacgcttccccatctcatcattctaatcatttgccaccagtacctgaagttccaggtgttccagttttgggtaatttgttgcaattgaaagaaaaaaagccttacatgaccttcaccaagtgggctgaaatgtatggtccaatctactctattagaactggtgctacttccatggttgttgtctcttctaacgaaatcgccaaagaagttgttgttaccagattcccatctatctctaccagaaaattgtcttacgccttgaaggttttgaccgaagataagtctatggttgccatgtctgattatcacgattaccataagaccgtcaagagacatattttgactgctgttttgggtccaaacgcccaaaaaaagtttagagcacatagagacaccatgatggaaaacgtttccaatgaattgcatgccttcttcgaaaagaacccaaatcaagaagtcaacttgagaaagatcttccaatcccaattattcggtttggctatgaagcaagccttgggtaaagatgttgaatccatctacgttaaggatttggaaaccaccatgaagagagaagaaatcttcgaagttttggttgtcgatccaatgatgggtgctattgaagttgattggagagactttttcccatacttgaaatgggttccaaacaagtccttcgaaaacatcatccatagaatgtacactagaagagaagctgttatgaaggccttgatccaagaacacaagaaaagaattgcctccggtgaaaacttgaactcctacattgattacttgttgtctgaagcccaaaccttgaccgataagcaattattgatgtctttgtgggaacctattatcgaatcttctgataccactatggttactactgaatgggctatgtacgaattggctaagaatccaaacatgcaagacagattatacgaagaaatccaatccgtttgcggttccgaaaagattactgaagaaaacttgtcccaattgccatacttgtacgctgttttccaagaaactttgagaaagcactgtccagttcctattatgccattgagatatgttcacgaaaacaccgttttgggtggttatcatgttccagctggtactgaagttgctattaacatctacggttgcaacatggataagaaggtctgggaaaatccagaagaatggaatccagaaagattcttgtccgaaaaagaatccatggacttgtacaaaactatggcttttggtggtggtaaaagagtttgcgctggttctttacaagccatggttatttcttgcattggtatcggtagattggtccaagattttgaatggaagttgaaggatgatgccgaagaagatgttaacactttgggtttgactacccaaaagttgcatccattattggccttgattaacccaagaaagtaactcgagccgcgg

SEQ ID NO: 62 - protein, 512 aa, Lactuca sativa
MDGVIDMQTIPLRTAIAIGGTAVALVVALYFWFLRSYASPSHHSNHLPPVPEVPGVPVLGNLLQLKEKKPYMTFTKWAEMYGPIYSIRTGATSMVVVSSNEIAKEVVVTRFPSISTRKLSYALKVLTEDKSMVAMSDYHDYHKTVKRHILTAVLGPNAQKKFRAHRDTMMENVSNELHAFFEKNPNQEVNLRKIFQSQLFGLAMKQALGKDVESIYVKDLETTMKREEIFEVLVVDPMMGAIEVDWRDFFPYLKWVPNKSFENIIHRMYTRREAVMKALIQEHKKRIASGENLNSYIDYLLSEAQTLTDKQLLMSLWEPIIESSDTTMVTTEWAMYELAKNPNMQDRLYEEIQSVCGSEKITEENLSQLPYLYAVFQETLRKHCPVPIMPLRYVHENTVLGGYHVPAGTEVAINIYGCNMDKKVWENPEEWNPERFLSEKESMDLYKTMAFGGGKRVCAGSLQAMVISCIGIGRLVQDFEWKLKDDAEEDVNTLGLTTQKLHPLLALINPRK

SEQ ID NO: 63 - DNA, 1535 bp, Rubus suavissimus
atggccaccctccttgagcatttccaagctatgccctttgccatccctattgcactggctgctctgtcttggctgttcctcttttacatcaaagtttcattcttttccaacaagagtgctcaggctaagctccctcctgtgccagtggttcctgggctgccggtgattgggaatttactgcaactcaaggagaagaaaccctaccagacttttacaaggtgggctgaggagtatggaccaatctattctatcaggactggtgcttccaccatggtcgttctcaataccacccaagttgcaaaagaggccatggtgaccagatatttatccatctcaaccagaaagctatcaaacgcactaaagattcttactgctgataaatgtatggttgcaataagtgactacaacgattttcacaagatgataaagcgatacatactctcaaatgttcttggacctagtgctcagaagcgtcaccggagcaacagagataccttgagagctaatgtctgcagccgattgcattctcaagtaaagaactctcctcgagaagctgtgaatttcagaagagtttttgagtgggaactctttggaattgcattgaagcaagcctttggaaaggacatagaaaagcccatttatgtggaggaacttggcactacactgtcaagagatgagatctttaaggttctagtgcttgacataatggagggtgcaattgaggttgattggagagatttcttcccttacctgagatggattccgaatacgcgcatggaaacaaaaattcagcgactctatttccgcaggaaagcagtgatgactgccctgatcaacgagcagaagaagcgaattgcttcaggagaggaaatcaactgttatatcgacttcttgcttaaggaagggaagacactgacaatggaccaaataagtatgttgctttgggagacggttattgaaacagcagatactacaatggtaacgacagaatgggctatgtatgaagttgctaaagactcaaagcgtcaggatcgtctctatcaggaaatccaaaaggtttgtggatcggagatggttacagaggaatacttgtcccaactgccgtacctgaatgcagttttccatgaaacgctaaggaagcacagtccggctgcgttagttcctttaagatatgcacatgaagatacccaactaggaggttactacattccagctggaactgagattgctataaacatatacgggtgtaacatggacaagcatcaatgggaaagccctgaggaatggaaaccggagagatttttggacccgaaatttgatcctatggatttgtacaagaccatggcttttggggctggaaagagggtatgtgctggttctcttcaggcaatgttaatagcgtgcccgacgattggtaggctggtgcaggagtttgagtggaagctgagagatggagaagaagaaaatgtagatactgttgggctcaccactcacaaacgctatccaatgcatgcaatcctgaagccaagaagtta

SEQ ID NO: 64 - DNA, 1536 bp, Artificial Sequence
  Codon-optimized KO
atggctaccttgttggaacattttcaagctatgccattcgctattccaattgctttggctgctttgtcttggttgtttttgttctacatcaaggtttctttcttctccaacaaatccgctcaagctaaattgccaccagttccagttgttccaggtttgccagttattggtaatttgttgcaattgaaagaaaagaagccataccaaaccttcactagatgggctgaagaatatggtccaatctactctattagaactggtgcttctactatggttgtcttgaacactactcaagttgccaaagaagctatggttaccagatacttgtctatctctaccagaaagttgtccaacgccttgaaaattttgaccgctgataagtgcatggttgccatttctgattacaacgatttccacaagatgatcaagagatatatcttgtctaacgttttgggtccatctgcccaaaaaagacatagatctaacagagataccttgagagccaacgtttgttctagattgcattcccaagttaagaactctccaagagaagctgtcaactttagaagagttttcgaatgggaattattcggtatcgctttgaaacaagccttcggtaaggatattgaaaagccaatctacgtcgaagaattgggtactactttgtccagagatgaaatcttcaaggttttggtcttggacattatggaaggtgccattgaagttgattggagagattttttcccatacttgcgttggattccaaacaccagaatggaaactaagatccaaagattatactttagaagaaaggccgttatgaccgccttgattaacgaacaaaagaaaagaattgcctccggtgaagaaatcaactgctacatcgatttcttgttgaaagaaggtaagaccttgaccatggaccaaatctctatgttgttgtgggaaaccgttattgaaactgctgataccacaatggttactactgaatgggctatgtacgaagttgctaaggattctaaaagacaagacagattataccaagaaatccaaaaggtctgcggttctgaaatggttacagaagaatacttgtcccaattgccatacttgaatgctgttttccacgaaactttgagaaaacattctccagctgctttggttccattgagatatgctcatgaagatactcaattgggtggttattacattccagccggtactgaaattgccattaacatctacggttgcaacatggacaaacaccaatgggaatctccagaagaatggaagccagaaagatttttggatcctaagtttgacccaatggacttgtacaaaactatggcttttggtgctggtaaaagagtttgcgctggttctttacaagctatgttgattgcttgtccaaccatcggtagattggttcaagaatttgaatggaagttgagagatggtgaagaagaaaacgttgatactgttggtttgaccacccataagagatatccaatgcatgctattttgaagccaagatcttaa

SEQ ID NO: 65 - DNA, 1572 bp, Artificial Sequence
  Codon-optimized KO
aagcttactagtaaaatggcctccatcacccatttcttacaagattttcaagctactccattcgctactgcttttgctgttggtggtgtttctttgttgatattcttcttcttcatccgtggtttccactctactaagaaaaacgaatattacaagttgccaccagttccagttgttccaggtttgccagttgttggtaatttgttgcaattgaaagaaaagaagccatacaagactttcttgagatgggctgaaattcatggtccaatctactctattagaactggtgcttctaccatggttgttgttaactctactcatgttgccaaagaagctatggttaccagattctcttcaatctctaccagaaagttgtccaaggctttggaattattgacctccaacaaatctatggttgccacctctgattacaacgaatttcacaagatggtcaagaagtacatcttggccgaattattgggtgctaatgctcaaaagagacacagaattcatagagacaccttgatcgaaaacgtcttgaacaaattgcatgcccataccaagaattctccattgcaagctgttaacttcagaaagatcttcgaatctgaattattcggtttggctatgaagcaagccttgggttatgatgttgattccttgttcgttgaagaattgggtactaccttgtccagagaagaaatctacaacgttttggtcagtgacatgttgaagggtgctattgaagttgattggagagactttttcccatacttgaaatggatcccaaacaagtccttcgaaatgaagattcaaagattggcctctagaagacaagccgttatgaactctattgtcaaagaacaaaagaagtccattgcctctggtaagggtgaaaactgttacttgaattacttgttgtccgaagctaagactttgaccgaaaagcaaatttccattttggcctgggaaaccattattgaaactgctgatacaactgttgttaccactgaatgggctatgtacgaattggctaaaaacccaaagcaacaagacagattatacaacgaaatccaaaacgtctgcggtactgataagattaccgaagaacatttgtccaagttgccttacttgtctgctgtttttcacgaaaccttgagaaagtattctccatctccattggttccattgagatacgctcatgaagatactcaattgggtggttattatgttccagccggtactgaaattgctgttaatatctacggttgcaacatggacaagaatcaatgggaaactccagaagaatggaagccagaaagatttttggacgaaaagtacgatccaatggacatgtacaagactatgtcttttggttccggtaaaagagtttgcgctggttctttacaagctagtttgattgcttgtacctccatcggtagattggttcaagaatttgaatggagattgaaagacggtgaagttgaaaacgttgataccttgggtttgactacccataagttgtatccaatgcaagctatcttgcaacctagaaactgactcgagccgcgg

SEQ ID NO: 66 - protein, 514 aa, Castanea mollissima
MASITHFLQDFQATPFATAFAVGGVSLLIFFFFIRGFHSTKKNEYYKLPPVPVVPGLPVVGNLLQLKEKKPYKTFLRWAEIHGPIYSIRTGASTMVVVNSTHVAKEAMVTRFSSISTRKLSKALELLTSNKSMVATSDYNEFHKMVKKYILAELLGANAQKRHRIHRDTLIENVLNKLHAHTKNSPLQAVNFRKIFESELFGLAMKQALGYDVDSLFVEELGTTLSREEIYNVLVSDMLKGAIEVDWRDFFPYLKWIPNKSFEMKIQRLASRRQAVMNSIVKEQKKSIASGKGENCYLNYLLSEAKTLTEKQISILAWETIIETADTTVVTTEWAMYELAKNPKQQDRLYNEIQNVCGTDKITEEHLSKLPYLSAVFHETLRKYSPSPLVPLRYAHEDTQLGGYYVPAGTEIAVNIYGCNMDKNQWETPEEWKPERFLDEKYDPMDMYKTMSFGSGKRVCAGSLQASLIACTSIGRLVQEFEWRLKDGEVENVDTLGLTTHKLYPMQAILQPRN

SEQ ID NO: 67 - DNA, 1512 bp, Artificial Sequence
  Codon-optimized KO
atgatttccttgttgttgggttttgttgtctcctccttcttgtttatcttcttcttgaaaaaattgttgttcttcttcagtcgtcacaaaatgtccgaagtttctagattgccatctgttccagttccaggttttccattgattggtaacttgttgcaattgaaagaaaagaagccacacaagactttcaccaagtggtctgaattatatggtccaatctactctatcaagatgggttcctcttctttgatcgtcttgaactctattgaaaccgccaaagaagctatggtcagtagattctcttcaatctctaccagaaagttgtctaacgctttgactgttttgacctgcaacaaatctatggttgctacctctgattacgatgactttcataagttcgtcaagagatgcttgttgaacggtttgttgggtgctaatgctcaagaaagaaaaagacattacagagatgccttgatcgaaaacgttacctctaaattgcatgcccataccagaaatcatccacaagaaccagttaacttcagagccattttcgaacacgaattattcggtgttgctttgaaacaagccttcggtaaagatgtcgaatccatctatgtaaaagaattgggtgtcaccttgtccagagatgaaattttcaaggttttggtccacgacatgatggaaggtgctattgatgttgattggagagatttcttcccatacttgaaatggatcccaaacaactctttcgaagccagaattcaacaaaagcacaagagaagattggctgttatgaacgccttgatccaagacagattgaatcaaaacgattccgaatccgatgatgactgctacttgaatttcttgatgtctgaagctaagaccttgaccatggaacaaattgctattttggtttgggaaaccattatcgaaactgctgataccactttggttactactgaatgggctatgtacgaattggccaaacatcaatctgttcaagatagattattcaaagaaatccaatccgtctgcggtggtgaaaagatcaaagaagaacaattgccaagattgccttacgtcaatggtgtttttcacgaaaccttgagaaagtattctccagctccattggttccaattagatacgctcatgaagatacccaaattggtggttatcatattccagccggttctgaaattgccattaacatctacggttgcaacatggataagaagagatgggaaagacctgaagaatggtggccagaaagatttttggaagatagatacgaatcctccgacttgcataagactatggcttttggtgctggtaaaagagtttgtgctggtgctttacaagctagtttgatggctggtattgctatcggtagattggttcaagaattcgaatggaagttgagagatggtgaagaagaaaacgttgatacttacggtttgacctcccaaaagttgtatccattgatggccattatcaacccaagaagatcttaa

SEQ ID NO: 68 - protein, 506 aa, Thellungiella halophila
MASMISLLLGFVVSSFLFIFFLKKLLFFFSRHKMSEVSRLPSVPVPGFPLIGNLLQLKEKKPHKTFTKWSELYGPIYSIKMGSSSLIVLNSIETAKEAMVSRFSSISTRKLSNALTVLTCNKSMVATSDYDDFHKFVKRCLLNGLLGANAQERKRHYRDALIENVTSKLHAHTRNHPQEPVNFRAIFEHELFGVALKQAFGKDVESIYVKELGVTLSRDEIFKVLVHDMMEGAIDVDWRDFFPYLKWIPNNSFEARIQQKHKRRLAVMNALIQDRLNQNDSESDDDCYLNFLMSEAKTLTMEQIAILVWETIIETADTTLVTTEWAMYELAKHQSVQDRLFKEIQSVCGGEKIKEEQLPRLPYVNGVFHETLRKYSPAPLVPIRYAHEDTQIGGYHIPAGSEIAINIYGCNMDKKRWERPEEWWPERFLEDRYESSDLHKTMAFGAGKRVCAGALQASLMAGIAIGRLVQEFEWKLRDGEEENVDTYGLTSQKLYPLMAIINPRRS

SEQ ID NO: 69 - DNA, 1554 bp, Artificial Sequence
  Codon-optimized KO
aagcttactagtaaaatggacatgatgggtattgaagctgttccatttgctactgctgttgttttgggtggtatttccttggttgttttgatcttcatcagaagattcgtttccaacagaaagagatccgttgaaggtttgccaccagttccagatattccaggtttaccattgattggtaacttgttgcaattgaaagaaaagaagccacataagacctttgctagatgggctgaaacttacggtccaattttctctattagaactggtgcttctaccatgatcgtcttgaattcttctgaagttgccaaagaagctatggtcactagattctcttcaatctctaccagaaagttgtccaacgccttgaagattttgaccttcgataagtgtatggttgccacctctgattacaacgattttcacaaaatggtcaagggtttcatcttgagaaacgttttaggtgctccagcccaaaaaagacatagatgtcatagagataccttgatcgaaaacatctctaagtacttgcatgcccatgttaagacttctccattggaaccagttgtcttgaagaagattttcgaatccgaaattttcggtttggctttgaaacaagccttgggtaaggatatcgaatccatctatgttgaagaattgggtactaccttgtccagagaagaaatttttgccgttttggttgttgatccaatggctggtgctattgaagttgattggagagattttttcccatacttgtcctggattccaaacaagtctatggaaatgaagatccaaagaatggattttagaagaggtgctttgatgaaggccttgattggtgaacaaaagaaaagaatcggttccggtgaagaaaagaactcctacattgatttcttgttgtctgaagctaccactttgaccgaaaagcaaattgctatgttgatctgggaaaccatcatcgaaatttccgatacaactttggttacctctgaatgggctatgtacgaattggctaaagacccaaatagacaagaaatcttgtacagagaaatccacaaggtttgcggttctaacaagttgactgaagaaaacttgtccaagttgccatacttgaactctgttttccacgaaaccttgagaaagtattctccagctccaatggttccagttagatatgctcatgaagatactcaattgggtggttaccatattccagctggttctcaaattgccattaacatctacggttgcaacatgaacaaaaagcaatgggaaaatcctgaagaatggaagccagaaagattcttggacgaaaagtatgacttgatggacttgcataagactatggcttttggtggtggtaaaagagtttgtgctggtgctttacaagcaatgttgattgcttgcacttccatcggtagattcgttcaagaatttgaatggaagttgatgggtggtgaagaagaaaacgttgatactgttgctttgacctcccaaaaattgcatccaatgcaagccattattaaggccagagaatgactcgagccgcgg

SEQ ID NO: 70 - protein, 508 aa, Vitis vinifera
MDMMGIEAVPFATAVVLGGISLVVLIFIRRFVSNRKRSVEGLPPVPDIPGLPLIGNLLQLKEKKPHKTFARWAETYGPIFSIRTGASTMIVLNSSEVAKEAMVTRFSSISTRKLSNALKILTFDKCMVATSDYNDFHKMVKGFILRNVLGAPAQKRHRCHRDTLIENISKYLHAHVKTSPLEPVVLKKIFESEIFGLALKQALGKDIESIYVEELGTTLSREEIFAVLVVDPMAGAIEVDWRDFFPYLSWIPNKSMEMKIQRMDFRRGALMKALIGEQKKRIGSGEEKNSYIDFLLSEATTLTEKQIAMLIWETIIEISDTTLVTSEWAMYELAKDPNRQEILYREIHKVCGSNKLTEENLSKLPYLNSVFHETLRKYSPAPMVPVRYAHEDTQLGGYHIPAGSQIAINIYGCNMNKKQWENPEEWKPERFLDEKYDLMDLHKTMAFGGGKRVCAGALQAMLIACTSIGRFVQEFEWKLMGGEEENVDTVALTSQKLHPMQAIIKARE

SEQ ID NO: 71 - DNA, 1593 bp, Artificial Sequence
  Codon-optimized KO
aagcttaaaatgagtaagtctaatagtatgaattctacatcacacgaaaccctttttcaacaattggtcttgggtttggaccgtatgccattgatggatgttcactggttgatctacgttgctttcggcgcatggttatgttcttatgtgatacatgttttatcatcttcctctacagtaaaagtgccagttgttggatacaggtctgtattcgaacctacatggttgcttagacttagattcgtctgggaaggtggctctatcataggtcaagggtacaataagtttaaagactctattttccaagttaggaaattgggaactgatattgtcattataccacctaactatattgatgaagtgagaaaattgtcacaggacaagactagatcagttgaacctttcattaatgattttgcaggtcaatacacaagaggcatggttttcttgcaatctgacttacaaaaccgtgttatacaacaaagactaactccaaaattggtttccttgaccaaggtcatgaaggaagagttggattatgctttaacaaaagagatgcctgatatgaaaaatgacgaatgggtagaagtagatatcagtagtataatggtgagattgatttccaggatctccgccagagtctttctagggcctgaacactgtcgtaaccaggaatggttgactactacagcagaatattcagaatcacttttcattacagggtttatcttaagagttgtacctcatatcttaagaccattcatcgcccctctattaccttcatacaggactctacttagaaacgtttcaagtggtagaagagtcatcggtgacatcataagatctcagcaaggggatggtaacgaagatatactttcctggatgagagatgctgccacaggagaggaaaagcaaatcgataacattgctcagagaatgttaattctttctttagcatcaatccacactactgcgatgaccatgacacatgccatgtacgatctatgtgcttgccctgagtacattgaaccattaagagatgaagttaaatctgttgttggggcttctggctgggacaagacagcgttaaacagatttcataagttggactccttcctaaaagagtcacaaagattcaacccagtattcttattgacattcaatagaatctaccatcaatctatgaccttatcagatggcactaacattccatctggaacacgtattgctgttccatcacacgcaatgttgcaagattctgcacatgtcccaggtccaaccccacctactgaatttgatggattcagatatagtaagatacgttctgatagtaactacgcacaaaagtacctattctccatgaccgattcttcaaacatggctttcggatacggcaagtatgcttgtccaggtagattttacgcgtctaatgagatgaaactaacattagccattttgttgctacaatttgagttcaaactaccagatggtaaaggtcgtcctagaaatatcactatcgattctgatatgattccagacccaagagctagactttgcgtcagaaaaagatcacttagagatgaatgaccgcgg

SEQ ID NO: 72 - protein, 525 aa, Gibberella fujikuroi
MSKSNSMNSTSHETLFQQLVLGLDRMPLMDVHWLIYVAFGAWLCSYVIHVLSSSSTVKVPVVGYRSVFEPTWLLRLRFVWEGGSIIGQGYNKFKDSIFQVRKLGTDIVIIPPNYIDEVRKLSQDKTRSVEPFINDFAGQYTRGMVFLQSDLQNRVIQQRLTPKLVSLTKVMKEELDYALTKEMPDMKNDEWVEVDISSIMVRLISRISARVFLGPEHCRNQEWLTTTAEYSESLFITGFILRVVPHILRPFIAPLLPSYRTLLRNVSSGRRVIGDIIRSQQGDGNEDILSWMRDAATGEEKQIDNIAQRMLILSLASIHTTAMTMTHAMYDLCACPEYIEPLRDEVKSVVGASGWDKTALNRFHKLDSFLKESQRFNPVFLLTFNRIYHQSMTLSDGTNIPSGTRIAVPSHAMLQDSAHVPGPTPPTEFDGFRYSKIRSDSNYAQKYLFSMTDSSNMAFGYGKYACPGRFYASNEMKLTLAILLLQFEFKLPDGKGRPRNITIDSDMIPDPRARLCVRKRSLRDE

SEQ ID NO: 73 - DNA, 1515 bp, Artificial Sequence
  Codon-optimized KO
aagcttaaaatggaagatcctactgtcttatatgcttgtcttgccattgcagttgcaactttcgttgttagatggtacagagatccattgagatccatcccaacagttggtggttccgatttgcctattctatcttacatcggcgcactaagatggacaagacgtggcagagagatacttcaagagggatatgatggctacagaggatctacattcaaaatcgcgatgttagaccgttggatcgtgatcgcaaatggtcctaaactagctgatgaagtcagacgtagaccagatgaagagttaaactttatggacggattaggagcattcgtccaaactaagtacaccttaggtgaagctattcataacgatccataccatgtcgatatcataagagaaaaactaacaagaggccttccagccgtgcttcctgatgtcattgaagagttgacacttgcggttagacagtacattccaacagaaggtgatgaatgggtgtccgtaaactgttcaaaggccgcaagagatattgttgctagagcttctaatagagtctttgtaggtttgcctgcttgcagaaaccaaggttacttagatttggcaatagactttacattgtctgttgtcaaggatagagccatcatcaatatgtttccagaattgttgaagccaatagttggcagagttgtaggtaacgccaccagaaatgttcgtagagctgttccttttgttgctccattggtggaggaaagacgtagacttatggaagagtacggtgaagactggtctgaaaaacctaatgatatgttacagtggataatggatgaagctgcatccagagatagttcagtgaaggcaatcgcagagagattgttaatggtgaacttcgcggctattcatacctcatcaaacactatcactcatgctttgtaccaccttgccgaaatgcctgaaactttgcaaccacttagagaagagatcgaaccattagtcaaagaggagggctggaccaaggctgctatgggaaaaatgtggtggttagattcatttctaagagaatctcaaagatacaatggcattaacatcgtatctttaactagaatggctgacaaagatattacattgagtgatggcacatttttgccaaaaggtactctagtggccgttccagcgtattctactcatagagatgatgctgtctacgctgatgccttagtattcgatcctttcagattctcacgtatgagagcgagagaaggtgaaggtacaaagcaccagttcgttaatacttcagtcgagtacgttccatttggtcacggaaagcatgcttgtccaggaagattcttcgccgcaaacgaattgaaagcaatgttggcttacattgttctaaactatgatgtaaagttgcctggtgacggtaaacgtccattgaacatgtattggggtccaacagttttgcctgcaccagcaggccaagtattgttcagaaagagacaagttagtctataaccgcgg

SEQ ID NO: 74 - protein, 499 aa, Trametes versicolor
MEDPTVLYACLAIAVATFVVRWYRDPLRSIPTVGGSDLPILSYIGALRWTRRGREILQEGYDGYRGSTFKIAMLDRWIVIANGPKLADEVRRRPDEELNFMDGLGAFVQTKYTLGEAIHNDPYHVDIIREKLTRGLPAVLPDVIEELTLAVRQYIPTEGDEWVSVNCSKAARDIVARASNRVFVGLPACRNQGYLDLAIDFTLSVVKDRAIINMFPELLKPIVGRVVGNATRNVRRAVPFVAPLVEERRRLMEEYGEDWSEKPNDMLQWIMDEAASRDSSVKAIAERLLMVNFAAIHTSSNTITHALYHLAEMPETLQPLREEIEPLVKEEGWTKAAMGKMWWLDSFLRESQRYNGINIVSLTRMADKDITLSDGTFLPKGTLVAVPAYSTHRDDAVYADALVFDPFRFSRMRAREGEGTKHQFVNTSVEYVPFGHGKHACPGRFFAANELKAMLAYIVLNYDVKLPGDGKRPLNMYWGPTVLPAPAGQVLFRKRQVSL

SEQ ID NO: 75 - DNA, 1530 bp, Artificial Sequence
  Codon-optimized KO
atggcatttttctctatgatttcaattttgttgggatttgttatttcttctttcatcttcatctttttcttcaaaaagttacttagttttagtaggaaaaacatgtcagaagtttctactttgccaagtgttccagtagtgcctggttttccagttattgggaatttgttgcaactaaaggagaaaaagcctcataaaactttcactagatggtcagagatatatggacctatctactctataaagatgggttcttcatctcttattgtattgaacagtacagaaactgctaaggaagcaatggtcactagattttcatcaatatctaccagaaaattgtcaaacgccctaacagttctaacctgcgataagtctatggtcgccacttctgattatgatgacttccacaaattagttaagagatgtttgctaaatggacttcttggtgctaatgctcaaaagagaaaaagacactacagagatgctttgattgaaaatgtgagttccaagctacatgcacacgctagagatcatccacaagagccagttaactttagagcaattttcgaacacgaattgtttggtgtagcattaaagcaagccttcggtaaagacgtagaatccatatacgtcaaggagttaggcgtaacattatcaaaagatgaaatctttaaggtgcttgtacatgatatgatggagggtgcaattgatgtagattggagagatttcttcccatatttgaaatggatccctaataagtcttttgaagctaggatacaacaaaagcacaagagaagactagctgttatgaacgcacttatacaggacagattgaagcaaaatgggtctgaatcagatgatgattgttaccttaacttcttaatgtctgaggctaaaacattgactaaggaacagatcgcaatccttgtctgggaaacaatcattgaaacagcagatactaccttagtcacaactgaatgggccatatacgagctagccaaacatccatctgtgcaagataggttgtgtaaggagatccagaacgtgtgtggtggagagaaattcaaggaagagcagttgtcacaagttccttaccttaacggcgttttccatgaaaccttgagaaaatactcacctgcaccattagttcctattagatacgcccacgaagatacacaaatcggtggctaccatgttccagctgggtccgaaattgctataaacatctacgggtgcaacatggacaaaaagagatgggaaagaccagaagattggtggccagaaagattcttagatgatggcaaatatgaaacatctgatttgcataaaacaatggctttcggagctggcaaaagagtgtgtgccggtgctctacaagcctccctaatggctggtatcgctattggtagattggtccaagagttcgaatggaaacttagagatggtgaagaggaaaatgtcgatacttatgggttaacatctcaaaagttatacccactaatggcaatcatcaatcctagaagatcctaa

SEQ ID NO: 76 - protein, 509 aa, Arabidopsis thaliana
MAFFSMISILLGFVISSFIFIFFFKKLLSFSRKNMSEVSTLPSVPVVPGFPVIGNLLQLKEKKPHKTFTRWSEIYGPIYSIKMGSSSLIVLNSTETAKEAMVTRFSSISTRKLSNALTVLTCDKSMVATSDYDDFHKLVKRCLLNGLLGANAQKRKRHYRDALIENVSSKLHAHARDHPQEPVNFRAIFEHELFGVALKQAFGKDVESIYVKELGVTLSKDEIFKVLVHDMMEGAIDVDWRDFFPYLKWIPNKSFEARIQQKHKRRLAVMNALIQDRLKQNGSESDDDCYLNFLMSEAKTLTKEQIAILVWETIIETADTTLVTTEWAIYELAKHPSVQDRLCKEIQNVCGGEKFKEEQLSQVPYLNGVFHETLRKYSPAPLVPIRYAHEDTQIGGYHVPAGSEIAINIYGCNMDKKRWERPEDWWPERFLDDGKYETSDLHKTMAFGAGKRVCAGALQASLMAGIAIGRLVQEFEWKLRDGEEENVDTYGLTSQKLYPLMAIINPRRS

SEQ ID NO: 77 - DNA, 2133 bp, Artificial Sequence
  Codon-optimized CPR
atgcaatcagattcagtcaaagtctctccatttgatttggtttccgctgctatgaatggcaaggcaatggaaaagttgaacgctagtgaatctgaagatccaacaacattgcctgcactaaagatgctagttgaaaatagagaattgttgacactgttcacaacttccttcgcagttcttattgggtgtcttgtatttctaatgtggagacgttcatcctctaaaaagctggtacaagatccagttccacaagttatcgttgtaaagaagaaagagaaggagtcagaggttgatgacgggaaaaagaaagtttctattttctacggcacacaaacaggaactgccgaaggttttgctaaagcattagtcgaggaagcaaaagtgagatatgaaaagacctctttcaaggttatcgatctagatgactacgctgcagatgatgatgaatatgaggaaaaactgaaaaaggaatccttagccttcttcttcttggccacatacggtgatggtgaacctactgataatgctgctaacttctacaagtggttcacagaaggcgacgataaaggtgaatggctgaaaaagttacaatacggagtatttggtttaggtaacagacaatatgaacatttcaacaagatcgctattgtagttgatgataaacttactgaaatgggagccaaaagattagtaccagtaggattaggggatgatgatcagtgtatagaagatgacttcaccgcctggaaggaattggtatggccagaattggatcaacttttaagggacgaagatgatacttctgtgactaccccatacactgcagccgtattggagtacagagtggtttaccatgataaaccagcagactcatatgctgaagatcaaacccatacaaacggtcatgttgttcatgatgcacagcatccttcaagatctaatgtggctttcaaaaaggaactacacacctctcaatcagataggtcttgtactcacttagaattcgatatttctcacacaggactgtcttacgaaactggcgatcacgttggcgtttattccgagaacttgtccgaagttgtcgatgaagcactaaaactgttagggttatcaccagacacatacttctcagtccatgctgataaggaggatgggacacctatcggtggtgcttcactaccaccaccttttcctccttgcacattgagagacgctctaaccagatacgcagatgtcttatcctcacctaaaaaggtagctttgctggcattggctgctcatgctagtgatcctagtgaagccgataggttaaagttcctggcttcaccagccggaaaagatgaatatgcacaatggatcgtcgccaaccaacgttctttgctagaagtgatgcaaagttttccatctgccaagcctccattaggtgtgttcttcgcagcagtagctccacgtttacaaccaagatactactctatcagttcatctcctaagatgtctcctaacagaatacatgttacatgtgctttggtgtacgagactactccagcaggcagaattcacagaggattgtgttcaacctggatgaaaaatgctgtccctttaacagagtcacctgattgctctcaagcatccattttcgttagaacatcaaatttcagacttccagtggatccaaaagttccagtcattatgataggaccaggcactggtcttgccccattcaggggctttcttcaagagagattggccttgaaggaatctggtacagaattgggttcttctatctttttctttggttgccgtaatagaaaagttgactttatctacgaggacgagcttaacaattttgttgagacaggagcattgtcagaattgatcgtcgcattttcaagagaagggactgccaaagagtacgttcagcacaagatgagtcaaaaagcctccgatatatggaaacttctaagtgaaggtgcctatctttatgtctgtggcgatgcaaagggcatggccaaggatgtccatagaactctgcatacaattgttcaggaacaagggagtctggattcttccaaggctgaattgtacgtcaaaaacttacagatgtctggaagatacttaagagatgtttggtaa

SEQ ID NO: 78 - protein, 710 aa, Stevia rebaudiana
MQSDSVKVSPFDLVSAAMNGKAMEKLNASESEDPTTLPALKMLVENRELLTLFTTSFAVLIGCLVFLMWRRSSSKKLVQDPVPQVIVVKKKEKESEVDDGKKKVSIFYGTQTGTAEGFAKALVEEAKVRYEKTSFKVIDLDDYAADDDEYEEKLKKESLAFFFLATYGDGEPTDNAANFYKWFTEGDDKGEWLKKLQYGVFGLGNRQYEHFNKIAIVVDDKLTEMGAKRLVPVGLGDDDQCIEDDFTAWKELVWPELDQLLRDEDDTSVTTPYTAAVLEYRVVYHDKPADSYAEDQTHTNGHVVHDAQHPSRSNVAFKKELHTSQSDRSCTHLEFDISHTGLSYETGDHVGVYSENLSEVVDEALKLLGLSPDTYFSVHADKEDGTPIGGASLPPPFPPCTLRDALTRYADVLSSPKKVALLALAAHASDPSEADRLKFLASPAGKDEYAQWIVANQRSLLEVMQSFPSAKPPLGVFFAAVAPRLQPRYYSISSSPKMSPNRIHVTCALVYETTPAGRIHRGLCSTWMKNAVPLTESPDCSQASIFVRTSNFRLPVDPKVPVIMIGPGTGLAPFRGFLQERLALKESGTELGSSIFFFGCRNRKVDFIYEDELNNFVETGALSELIVAFSREGTAKEYVQHKMSQKASDIWKLLSEGAYLYVCGDAKGMAKDVHRTLHTIVQEQGSLDSSKAELYVKNLQMSGRYLRDVW

SEQ ID NO: 79 - DNA, 2106 bp, Siraitia grosvenorii
atgaaggtcagtccattcgaattcatgtccgctattatcaagggtagaatggacccatctaactcctcatttgaatctactggtgaagttgcctccgttatctttgaaaacagagaattggttgccatcttgaccacttctattgctgttatgattggttgcttcgttgtcttgatgtggagaagagctggttctagaaaggttaagaatgtcgaattgccaaagccattgattgtccatgaaccagaacctgaagttgaagatggtaagaagaaggtttccatcttcttcggtactcaaactggtactgctgaaggttttgctaaggctttggctgatgaagctaaagctagatacgaaaaggctaccttcagagttgttgatttggatgattatgctgccgatgatgaccaatacgaagaaaaattgaagaacgaatccttcgccgttttcttgttggctacttatggtgatggtgaacctactgataatgctgctagattttacaagtggttcgccgaaggtaaagaaagaggtgaatggttgcaaaacttgcactatgctgtttttggtttgggtaacagacaatacgaacacttcaacaagattgctaaggttgccgacgaattattggaagctcaaggtggtaatagattggttaaggttggtttaggtgatgacgatcaatgcatcgaagatgatttttctgcttggagagaatctttgtggccagaattggatatgttgttgagagatgaagatgatgctactactgttactactccatatactgctgctgtcttggaatacagagttgtctttcatgattctgctgatgttgctgctgaagataagtcttggattaacgctaatggtcatgctgttcatgatgctcaacatccattcagatctaacgttgtcgtcagaaaagaattgcatacttctgcctctgatagatcctgttctcatttggaattcaacatttccggttccgctttgaattacgaaactggtgatcatgttggtgtctactgtgaaaacttgactgaaactgttgatgaagccttgaacttgttgggtttgtctccagaaacttacttctctatctacaccgataacgaagatggtactccattgggtggttcttcattgccaccaccatttccatcatgtactttgagaactgctttgaccagatacgctgatttgttgaactctccaaaaaagtctgctttgttggctttagctgctcatgcttctaatccagttgaagctgatagattgagatacttggcttctccagctggtaaagatgaatatgcccaatctgttatcggttcccaaaagtctttgttggaagttatggctgaattcccatctgctaaaccaccattaggtgttttttttgctgctgttgctccaagattgcaacctagattctactccatttcatcctctccaagaatggctccatctagaatccatgttacttgtgctttggtttacgataagatgccaactggtagaattcataagggtgtttgttctacctggatgaagaattctgttccaatggaaaagtcccatgaatgttcttgggctccaattttcgttagacaatccaattttaagttgccagccgaatccaaggttccaattatcatggttggtccaggtactggtttggctccttttagaggttttttacaagaaagattggccttgaaagaatccggtgttgaattgggtccatccattttgtttttcggttgcagaaacagaagaatggattacatctacgaagatgaattgaacaacttcgttgaaaccggtgctttgtccgaattggttattgctttttctagagaaggtcctaccaaagaatacgtccaacataagatggctgaaaaggcttctgatatctggaacttgatttctgaaggtgcttacttgtacgtttgtggtgatgctaaaggtatggctaaggatgttcatagaaccttgcataccatcatgcaagaacaaggttctttggattcttccaaagctgaatccatggtcaagaacttgcaaatgaatggtagatacttaagagatgtttggtaa

SEQ ID NO: 80 - protein, 701 aa, Siraitia grosvenorii
MKVSPFEFMSAIIKGRMDPSNSSFESTGEVASVIFENRELVAILTTSIAVMIGCFVVLMWRRAGSRKVKNVELPKPLIVHEPEPEVEDGKKKVSIFFGTQTGTAEGFAKALADEAKARYEKATFRVVDLDDYAADDDQYEEKLKNESFAVFLLATYGDGEPTDNAARFYKWFAEGKERGEWLQNLHYAVFGLGNRQYEHFNKIAKVADELLEAQGGNRLVKVGLGDDDQCIEDDFSAWRESLWPELDMLLRDEDDATTVTTPYTAAVLEYRVVFHDSADVAAEDKSWINANGHAVHDAQHPFRSNVVVRKELHTSASDRSCSHLEFNISGSALNYETGDHVGVYCENLTETVDEALNLLGLSPETYFSIYTDNEDGTPLGGSSLPPPFPSCTLRTALTRYADLLNSPKKSALLALAAHASNPVEADRLRYLASPAGKDEYAQSVIGSQKSLLEVMAEFPSAKPPLGVFFAAVAPRLQPRFYSISSSPRMAPSRIHVTCALVYDKMPTGRIHKGVCSTWMKNSVPMEKSHECSWAPIFVRQSNFKLPAESKVPIIMVGPGTGLAPFRGFLQERLALKESGVELGPSILFFGCRNRRMDYIYEDELNNFVETGALSELVIAFSREGPTKEYVQHKMAEKASDIWNLISEGAYLYVCGDAKGMAKDVHRTLHTIMQEQGSLDSSKAESMVKNLQMNGRYLRDVW

SEQ ID NO: 81 - DNA, 2142 bp, Artificial Sequence
  Codon-optimized CPR
atggcagaattagatacacttgatatagtagtattaggtgttatctttttgggtactgtggcatactttactaagggtaaattgtggggtgttaccaaggatccatacgctaacggattcgctgcaggtggtgcttccaagcctggcagaactagaaacatcgtcgaagctatggaggaatcaggtaaaaactgtgttgttttctacggcagtcaaacaggtacagcggaggattacgcatcaagacttgcaaaggaaggaaagtccagattcggtttgaacactatgatcgccgatctagaagattatgacttcgataacttagacactgttccatctgataacatcgttatgtttgtattggctacttacggtgaaggcgaaccaacagataacgccgtggatttctatgagttcattactggcgaagatgcctctttcaatgagggcaacgatcctccactaggtaacttgaattacgttgcgttcggtctgggcaacaatacctacgaacactacaactcaatggtcaggaacgttaacaaggctctagaaaagttaggagctcatagaattggagaagcaggtgagggtgacgacggagctggaactatggaagaggactttttagcttggaaagatccaatgtgggaagccttggctaaaaagatgggcttggaggaaagagaagctgtatatgaacctattttcgctatcaatgagagagatgatttgacccctgaagcgaatgaggtatacttgggagaacctaataagctacacttggaaggtacagcgaaaggtccattcaactcccacaacccatatatcgcaccaattgcagaatcatacgaacttttctcagctaaggatagaaattgtctgcatatggaaattgatatttctggtagtaatctaaagtatgaaacaggcgaccatatcgcgatctggcctaccaacccaggtgaagaggtcaacaaatttcttgacattctagatctgtctggtaagcaacattccgtcgtaacagtgaaagccttagaacctacagccaaagttccttttccaaatccaactacctacgatgctatattgagataccatctggaaatatgcgctccagtttctagacagtttgtctcaactttagcagcattcgcccctaatgatgatatcaaagctgagatgaaccgtttgggatcagacaaagattacttccacgaaaagacaggaccacattactacaatatcgctagatttttggcctcagtctctaaaggtgaaaaatggacaaagataccattttctgctttcatagaaggccttacaaaactacaaccaagatactattctatctcttcctctagtttagttcagcctaaaaagattagtattactgctgttgtcgaatctcagcaaattccaggtagagatgacccattcagaggtgtagcgactaactacttgttcgctttgaagcagaaacaaaacggtgatccaaatccagctccttttggccaatcatacgagttgacaggaccaaggaataagtatgatggtatacatgttccagtccatgtaagacattctaactttaagctaccatctgatccaggcaaacctattatcatgatcggtccaggtaccggtgttgccccttttagaggcttcgtccaagagagggcaaaacaagccagagatggtgtagaagttggtaaaacactgctgttctttggatgtagaaagagtacagaagatttcatgtatcaaaaagagtggcaagagtacaaggaagctcttggcgacaaattcgaaatgattacagctttttcaagagaaggatctaaaaaggtttatgttcaacacagactgaaggaaagatcaaaggaagtttctgatcttctatcccaaaaagcatacttctacgtttgcggagacgccgcacatatggcacgtgaagtgaacactgtgttagcacagatcatagcagaaggccgtggtgtatcagaagccaagggtgaggaaattgtcaaaaacatgagatcagcaaatcaataccaagtgtgttctgatttcgtaactttacactgtaaagagacaacatacgcgaattcagaattgcaagaggatgtctggagttaa

SEQ ID NO: 82 - protein, 713 aa, Gibberella fujikuroi
MAELDTLDIVVLGVIFLGTVAYFTKGKLWGVTKDPYANGFAAGGASKPGRTRNIVEAMEESGKNCVVFYGSQTGTAEDYASRLAKEGKSRFGLNTMIADLEDYDFDNLDTVPSDNIVMFVLATYGEGEPTDNAVDFYEFITGEDASFNEGNDPPLGNLNYVAFGLGNNTYEHYNSMVRNVNKALEKLGAHRIGEAGEGDDGAGTMEEDFLAWKDPMWEALAKKMGLEEREAVYEPIFAINERDDLTPEANEVYLGEPNKLHLEGTAKGPFNSHNPYIAPIAESYELFSAKDRNCLHMEIDISGSNLKYETGDHIAIWPTNPGEEVNKFLDILDLSGKQHSVVTVKALEPTAKVPFPNPTTYDAILRYHLEICAPVSRQFVSTLAAFAPNDDIKAEMNRLGSDKDYFHEKTGPHYYNIARFLASVSKGEKWTKIPFSAFIEGLTKLQPRYYSISSSSLVQPKKISITAVVESQQIPGRDDPFRGVATNYLFALKQKQNGDPNPAPFGQSYELTGPRNKYDGIHVPVHVRHSNFKLPSDPGKPIIMIGPGTGVAPFRGFVQERAKQARDGVEVGKTLLFFGCRKSTEDFMYQKEWQEYKEALGDKFEMITAFSREGSKKVYVQHRLKERSKEVSDLLSQKAYFYVCGDAAHMAREVNTVLAQIIAEGRGVSEAKGEEIVKNMRSANQYQVCSDFVTLHCKETTYANSELQEDVWS

SEQ ID NO: 83 - DNA, 2130 bp, Stevia rebaudiana
atgcaatcggaatccgttgaagcatcgacgattgatttgatgactgctgttttgaaggacacagtgatcgatacagcgaacgcatctgataacggagactcaaagatgccgccggcgttggcgatgatgttcgaaattcgtgatctgttgctgattttgactacgtcagttgctgttttggtcggatgtttcgttgttttggtgtggaagagatcgtccgggaagaagtccggcaaggaattggagccgccgaagatcgttgtgccgaagaggcggctggagcaggaggttgatgatggtaagaagaaggttacgattttcttcggaacacaaactggaacggctgaaggtttcgctaaggcacttttcgaagaagcgaaagcgcgatatgaaaaggcagcgtttaaagtgattgatttggatgattatgctgctgatttggatgagtatgcagagaagctgaagaaggaaacatatgctttcttcttcttggctacatatggagatggtgagccaactgataatgctgccaaattttataaatggtttactgagggagacgagaaaggcgtttggcttcaaaaacttcaatatggagtatttggtcttggcaacagacaatatgaacatttcaacaagattggaatagtggttgatgatggtctcaccgagcagggtgcaaaacgcattgttcccgttggtcttggagacgacgatcaatcaattgaagacgatttttcggcatggaaagagttagtgtggcccgaattggatctattgcttcgcgatgaagatgacaaagctgctgcaactccttacacagctgcaatccctgaataccgcgtcgtatttcatgacaaacccgatgcgttttctgatgatcatactcaaaccaatggtcatgctgttcatgatgctcaacatccatgcagatccaatgtggctgttaaaaaagagcttcatactcctgaatccgatcgttcatgcacacatcttgaatttgacatttctcacactggattatcttatgaaactggggatcatgttggtgtatactgtgaaaacctaattgaagtagtggaagaagctgggaaattgttaggattatcaacagatacttatttctcgttacatattgataacgaagatggttcaccacttggtggaccttcattacaacctccttttcctccttgtactttaagaaaagcattgactaattatgcagatctgttaagctctcccaaaaagtcaactttgcttgctctagctgctcatgcttccgatcccactgaagctgatcgtttaagatttcttgcatctcgcgagggcaaggatgaatatgctgaatgggttgttgcaaaccaaagaagtcttcttgaagtcatggaagctttcccgtcagctagaccgccacttggtgttttctttgcagcggttgcaccgcgtttacagcctcgttactactctatttcttcctccccaaagatggaaccaaacaggattcatgttacttgcgcgttggtttatgaaaaaactcccgcaggtcgtatccacaaaggaatctgctcaacctggatgaagaacgctgtacctttgaccgaaagtcaagattgcagttgggcaccgatttttgttagaacatcaaacttcagacttccaattgacccgaaagtcccggttatcatgattggtcctggaaccgggttggctccatttaggggttttcttcaagaaagattggctcttaaagaatccggaaccgaactcgggtcatctattttattcttcggttgtagaaaccgcaaagtggattacatatatgagaatgaactcaacaactttgttgaaaatggtgcgctttctgagcttgatgttgctttctcccgcgatggcccgacgaaagaatacgtgcaacataaaatgacccaaaaggcttctgaaatatggaatatgctttctgagggagcatatttatatgtatgtggtgatgctaaaggcatggctaaagatgtacaccgtacacttcacaccattgtgcaagaacagggaagtttggactcgtctaaagcggagttgtatgtgaagaatctacaaatgtcaggaagatacctccgtgatgtttggtaa

SEQ ID NO: 84 - protein, 709 aa, Stevia rebaudiana
MQSESVEASTIDLMTAVLKDTVIDTANASDNGDSKMPPALAMMFEIRDLLLILTTSVAVLVGCFVVLVWKRSSGKKSGKELEPPKIVVPKRRLEQEVDDGKKKVTIFFGTQTGTAEGFAKALFEEAKARYEKAAFKVIDLDDYAADLDEYAEKLKKETYAFFFLATYGDGEPTDNAAKFYKWFTEGDEKGVWLQKLQYGVFGLGNRQYEHFNKIGIVVDDGLTEQGAKRIVPVGLGDDDQSIEDDFSAWKELVWPELDLLLRDEDDKAAATPYTAAIPEYRVVFHDKPDAFSDDHTQTNGHAVHDAQHPCRSNVAVKKELHTPESDRSCTHLEFDISHTGLSYETGDHVGVYCENLIEVVEEAGKLLGLSTDTYFSLHIDNEDGSPLGGPSLQPPFPPCTLRKALTNYADLLSSPKKSTLLALAAHASDPTEADRLRFLASREGKDEYAEWVVANQRSLLEVMEAFPSARPPLGVFFAAVAPRLQPRYYSISSSPKMEPNRIHVTCALVYEKTPAGRIHKGICSTWMKNAVPLTESQDCSWAPIFVRTSNFRLPIDPKVPVIMIGPGTGLAPFRGFLQERLALKESGTELGSSILFFGCRNRKVDYIYENELNNFVENGALSELDVAFSRDGPTKEYVQHKMTQKASEIWNMLSEGAYLYVCGDAKGMAKDVHRTLHTIVQEQGSLDSSKAELYVKNLQMSGRYLRDVW

SEQ ID NO: 85 - DNA, 2124 bp, Artificial Sequence
  Codon-optimized CPR
atgcaatctaactccgtgaagatttcgccgcttgatctggtaactgcgctgtttagcggcaaggttttggacacatcgaacgcatcggaatcgggagaatctgctatgctgccgactatagcgatgattatggagaatcgtgagctgttgatgatactcacaacgtcggttgctgtattgatcggatgcgttgtcgttttggtgtggcggagatcgtctacgaagaagtcggcgttggagccaccggtgattgtggttccgaagagagtgcaagaggaggaagttgatgatggtaagaagaaagttacggttttcttcggcacccaaactggaacagctgaaggcttcgctaaggcacttgttgaggaagctaaagctcgatatgaaaaggctgtctttaaagtaattgatttggatgattatgctgctgatgacgatgagtatgaggagaaactaaagaaagaatctttggcctttttctttttggctacgtatggagatggtgagccaacagataatgctgccagattttataaatggtttactgagggagatgcgaaaggagaatggcttaataagcttcaatatggagtatttggtttgggtaacagacaatatgaacattttaacaagatcgcaaaagtggttgatgatggtcttgtagaacagggtgcaaagcgtcttgttcctgttggacttggagatgatgatcaatgtattgaagatgacttcaccgcatggaaagagttagtatggccggagttggatcaattacttcgtgatgaggatgacacaactgttgctactccatacacagctgctgttgcagaatatcgcgttgtttttcatgaaaaaccagacgcgctttctgaagattatagttatacaaatggccatgctgttcatgatgctcaacatccatgcagatccaacgtggctgtcaaaaaggaacttcatagtcctgaatctgaccggtcttgcactcatcttgaatttgacatctcgaacaccggactatcatatgaaactggggaccatgttggagtttactgtgaaaacttgagtgaagttgtgaatgatgctgaaagattagtaggattaccaccagacacttactcctccatccacactgatagtgaagacgggtcgccacttggcggagcctcattgccgcctcctttcccgccatgcactttaaggaaagcattgacgtgttatgctgatgttttgagttctcccaagaagtcggctttgcttgcactagctgctcatgccaccgatcccagtgaagctgatagattgaaatttcttgcatcccccgccggaaaggatgaatattctcaatggatagttgcaagccaaagaagtctccttgaagtcatggaagcattcccgtcagctaagccttcacttggtgttttctttgcatctgttgccccgcgcttacaaccaagatactactctatttcttcctcacccaagatggcaccggataggattcatgttacatgtgcattagtctatgagaaaacacctgcaggccgcatccacaaaggagtttgttcaacttggatgaagaacgcagtgcctatgaccgagagtcaagattgcagttgggccccaatatacgtccgaacatccaatttcagactaccatctgaccctaaggtcccggttatcatgattggacctggcactggtttggctccttttagaggtttccttcaagagcggttagctttaaaggaagccggaactgacctcggtttatccattttattcttcggatgtaggaatcgcaaagtggatttcatatatgaaaacgagcttaacaactttgtggagactggtgctctttctgagcttattgttgctttctcccgtgaaggcccgactaaggaatatgtgcaacacaagatgagtgagaaggcttcggatatctggaacttgctttctgaaggagcatatttatacgtatgtggtgatgccaaaggcatggccaaagatgtacatcgaaccctccacacaattgtgcaagaacagggatctcttgactcgtcaaaggcagaactctacgtgaagaatctacaaatgtcaggaagatacctccgtgacgtttggtaa

SEQ ID NO: 86 - protein, 707 aa, Stevia rebaudiana
MQSNSVKISPLDLVTALFSGKVLDTSNASESGESAMLPTIAMIMENRELLMILTTSVAVLIGCVVVLVWRRSSTKKSALEPPVIVVPKRVQEEEVDDGKKKVTVFFGTQTGTAEGFAKALVEEAKARYEKAVFKVIDLDDYAADDDEYEEKLKKESLAFFFLATYGDGEPTDNAARFYKWFTEGDAKGEWLNKLQYGVFGLGNRQYEHFNKIAKVVDDGLVEQGAKRLVPVGLGDDDQCIEDDFTAWKELVWPELDQLLRDEDDTTVATPYTAAVAEYRVVFHEKPDALSEDYSYTNGHAVHDAQHPCRSNVAVKKELHSPESDRSCTHLEFDISNTGLSYETGDHVGVYCENLSEVVNDAERLVGLPPDTYSSIHTDSEDGSPLGGASLPPPFPPCTLRKALTCYADVLSSPKKSALLALAAHATDPSEADRLKFLASPAGKDEYSQWIVASQRSLLEVMEAFPSAKPSLGVFFASVAPRLQPRYYSISSSPKMAPDRIHVTCALVYEKTPAGRIHKGVCSTWMKNAVPMTESQDCSWAPIYVRTSNFRLPSDPKVPVIMIGPGTGLAPFRGFLQERLALKEAGTDLGLSILFFGCRNRKVDFIYENELNNFVETGALSELIVAFSREGPTKEYVQHKMSEKASDIWNLLSEGAYLYVCGDAKGMAKDVHRTLHTIVQEQGSLDSSKAELYVKNLQMSGRYLRDVW

SEQ ID NO: 87 - DNA, 2070 bp, Artificial Sequence
  Codon-optimized CPR
atgtcctccaactccgatttggtcagaagattggaatctgttttgggtgtttctttcggtggttctgttactgattccgttgttgttattgctaccacctctattgctttggttatcggtgttttggttttgttgtggagaagatcctctgacagatctagagaagttaagcaattggctgttccaaagccagttactatcgttgaagaagaagatgaattcgaagttgcttctggtaagaccagagtttctattttctacggtactcaaactggtactgctgaaggttttgctaaggctttggctgaagaaatcaaagccagatacgaaaaagctgccgttaaggttattgatttggatgattacacagccgaagatgacaaatacggtgaaaagttgaagaaagaaactatggccttcttcatgttggctacttatggtgatggtgaacctactgataatgctgctagattttacaagtggttcaccgaaggtactgatagaggtgtttggttggaacatttgagatacggtgtattcggtttgggtaacagacaatacgaacacttcaacaagattgccaaggttgttgatgatttgttggttgaacaaggtgccaagagattggttactgttggtttgggtgatgatgatcaatgcatcgaagatgatttctccgcttggaaagaagccttgtggccagaattggatcaattattgcaagatgataccaacaccgtttctactccatacactgctgttattccagaatacagagttgttatccacgatccatctgttacctcttatgaagatccatactctaacatggctaacggtaatgcctcttacgatattcatcatccatgtagagctaacgttgccgtccaaaaagaattgcataagccagaatctgacagaagttgcatccatttggaattcgatattttcgctactggtttgacttacgaaaccggtgatcatgttggtgtttacgctgataattgtgatgatactgtagaagaagccgctaagttgttgggtcaaccattggatttgttgttctccattcataccgataacaacgacggtacttctttgggttcttctttgccaccaccatttccaggtccatgtactttgagaactgctttggctagatatgccgatttgttgaatccaccaaaaaaggctgctttgattgctttagctgctcatgctgatgaaccatctgaagctgaaagattgaagttcttgtcatctccacaaggtaaggacgaatattctaaatgggttgtcggttcccaaagatccttggttgaagttatggctgaatttccatctgctaaaccaccattgggtgtattttttgctgctgttgttcctagattgcaacctagatattactccatctcttccagtccaagatttgctccacatagagttcatgttacttgcgctttggtttatggtccaactccaactggtagaattcacagaggtgtatgttcattctggatgaagaatgttgtcccattggaaaagtctcaaaactgttcttgggccccaattttcatcagacaatctaatttcaagttgccagccgatcattctgttccaatagttatggttggtccaggtactggtttagctccttttagaggtttcttacaagaaagattggccttgaaagaagaaggtgctcaagttggtcctgctttgttgttttttggttgcagaaacagacaaatggacttcatctacgaagtcgaattgaacaactttgtcgaacaaggtgctttgtccgaattgatcgttgctttttcaagagaaggtccatccaaagaatacgtccaacataagatggttgaaaaggcagcttacatgtggaacttgatttctcaaggtggttacttctacgtttgtggtgatgctaaaggtatggctagagatgttcatagaacattgcataccatcgtccaacaagaagaaaaggttgattctaccaaggccgaatccatcgttaagaaattgcaaatggacggtagatacttgagagatgtttggtga

SEQ ID NO: 88 - protein, 689 aa, Rubus suavissimus
MSSNSDLVRRLESVLGVSFGGSVTDSVVVIATTSIALVIGVLVLLWRRSSDRSREVKQLAVPKPVTIVEEEDEFEVASGKTRVSIFYGTQTGTAEGFAKALAEEIKARYEKAAVKVIDLDDYTAEDDKYGEKLKKETMAFFMLATYGDGEPTDNAARFYKWFTEGTDRGVWLEHLRYGVFGLGNRQYEHFNKIAKVVDDLLVEQGAKRLVTVGLGDDDQCIEDDFSAWKEALWPELDQLLQDDTNTVSTPYTAVIPEYRVVIHDPSVTSYEDPYSNMANGNASYDIHHPCRANVAVQKELHKPESDRSCIHLEFDIFATGLTYETGDHVGVYADNCDDTVEEAAKLLGQPLDLLFSIHTDNNDGTSLGSSLPPPFPGPCTLRTALARYADLLNPPKKAALIALAAHADEPSEAERLKFLSSPQGKDEYSKWVVGSQRSLVEVMAEFPSAKPPLGVFFAAVVPRLQPRYYSISSSPRFAPHRVHVTCALVYGPTPTGRIHRGVCSFWMKNVVPLEKSQNCSWAPIFIRQSNFKLPADHSVPIVMVGPGTGLAPFRGFLQERLALKEEGAQVGPALLFFGCRNRQMDFIYEVELNNFVEQGALSELIVAFSREGPSKEYVQHKMVEKAAYMWNLISQGGYFYVCGDAKGMARDVHRTLHTIVQQEEKVDSTKAESIVKKLQMDGRYLRDVW

SEQ ID NO: 89 - DNA, 2079 bp, Artificial Sequence
  Codon-optimized CPR
atgacttctgcactttatgcctccgatcttttcaaacaattgaaaagtatcatgggaacggattctttgtccgatgatgttgtattagttattgctacaacttctctggcactggttgctggtttcgttgtcttattgtggaaaaagaccacggcagatcgttccggcgagctaaagccactaatgatccctaagtctctgatggcgaaagatgaggatgatgacttagatctaggttctggaaaaacgagagtctctatcttcttcggcacacaaaccggaacagccgaaggattcgctaaagcactttcagaagagatcaaagcaagatacgaaaaggcggctgtaaaagtaatcgatttggatgattacgctgccgatgatgaccaatatgaggaaaagttgaaaaaggaaacattggctttcttttgtgtagccacgtatggtgatggtgaaccaaccgataacgccgcaagattctacaagtggtttactgaagagaacgaaagagatatcaagttgcagcaacttgcttacggcgtttttgccttaggtaacagacaatacgagcactttaacaagataggtattgtcttagatgaagagttatgcaaaaagggtgcgaagagattgattgaagtcggtttaggagatgatgatcaatctatcgaggatgactttaatgcatggaaggaatctttgtggtctgaattagataagttacttaaggacgaagatgataaatccgttgccactccatacacagccgtcattccagaatatagagtagttactcatgatccaagattcacaacacagaaatcaatggaaagtaatgtggctaatggtaatactaccatcgatattcatcatccatgtagagtagacgttgcagttcaaaaggaattgcacactcatgaatcagacagatcttgcatacatcttgaatttgatatatcacgtactggtatcacttacgaaacaggtgatcacgtgggtgtctacgctgaaaaccatgttgaaattgtagaggaagctggaaagttgttgggccatagtttagatcttgttttctcaattcatgccgataaagaggatggctcaccactagaaagtgcagtgcctccaccatttccaggaccatgcaccctaggtaccggtttagctcgttacgcggatctgttaaatcctccacgtaaatcagctctagtggccttggctgcgtacgccacagaaccttctgaggcagaaaaactgaaacatctaacttcaccagatggtaaggatgaatactcacaatggatagtagctagtcaacgttctttactagaagttatggctgctttcccatccgctaaacctcctttgggtgttttcttcgccgcaatagcgcctagactgcaaccaagatactattcaatttcatcctcacctagactggcaccatcaagagttcatgtcacatccgctttagtgtacggtccaactcctactggtagaatccataagggcgtttgttcaacatggatgaaaaacgcggttccagcagagaagtctcacgaatgttctggtgctccaatctttatcagagcctccaacttcaaactgccttccaatccttctactcctattgtcatggtcggtcctggtacaggtcttgctccattcagaggtttcttacaagagagaatggccttaaaggaggatggtgaagagttgggatcttctttgttgtttttcggctgtagaaacagacaaatggatttcatctacgaagatgaactgaataactttgtagatcaaggagttatttcagagttgataatggctttttctagagaaggtgctcagaaggagtacgtccaacacaaaatgatggaaaaggccgcacaagtttgggacttaatcaaagaggaaggctatctatatgtctgtggtgatgcaaagggtatggcaagagatgttcacagaacacttcatactatagtccaggaacaggaaggcgttagttcttctgaagcggaagcaattgtgaaaaagttacaaacagagggaagatacttgagagatgtgtggtaa

SEQ ID NO: 90 - protein, 692 aa, Arabidopsis thaliana [sequence omitted]

SEQ ID NO: 91 - DNA, 2139 bp, Artificial Sequence
  Codon-optimized CPR
atgtcttcctcttcctcttccagtacctctatgattgatttgatggctgctattattaaaggtgaaccagttatcgtctccgacccagcaaatgcctctgcttatgaatcagttgctgcagaattgtcttcaatgttgatcgaaaacagacaattcgccatgatcgtaactacatcaatcgctgttttgatcggttgtattgtcatgttggtatggagaagatccggtagtggtaattctaaaagagtcgaacctttgaaaccattagtaattaagccaagagaagaagaaatagatgacggtagaaagaaagttacaatatttttcggtacccaaactggtacagctgaaggttttgcaaaagccttaggtgaagaagctaaggcaagatacgaaaagactagattcaagatagtcgatttggatgactatgccgctgatgacgatgaatacgaagaaaagttgaagaaagaagatgttgcatttttctttttggcaacctatggtgacggtgaaccaactgacaatgcagccagattctacaaatggtttacagagggtaatgatcgtggtgaatggttgaaaaacttaaagtacggtgttttcggtttgggtaacagacaatacgaacatttcaacaaagttgcaaaggttgtcgacgatattttggtcgaacaaggtgctcaaagattagtccaagtaggtttgggtgacgatgaccaatgtatagaagatgactttactgcctggagagaagctttgtggcctgaattagacacaatcttgagagaagaaggtgacaccgccgttgctaccccatatactgctgcagtattagaatacagagtttccatccatgatagtgaagacgcaaagtttaatgatatcactttggccaatggtaacggttatacagttttcgatgcacaacacccttacaaagctaacgttgcagtcaagagagaattacatacaccagaatccgacagaagttgtatacacttggaatttgatatcgctggttccggtttaaccatgaagttgggtgaccatgtaggtgttttatgcgacaatttgtctgaaactgttgatgaagcattgagattgttggatatgtcccctgacacttattttagtttgcacgctgaaaaagaagatggtacaccaatttccagttctttaccacctccattccctccatgtaacttaagaacagccttgaccagatacgcttgcttgttatcatcccctaaaaagtccgccttggttgctttagccgctcatgctagtgatcctactgaagcagaaagattgaaacacttagcatctccagccggtaaagatgaatattcaaagtgggtagttgaatctcaaagatcattgttagaagttatggcagaatttccatctgccaagcctccattaggtgtcttctttgctggtgtagcacctagattgcaaccaagattctactcaatcagttcttcacctaagatcgctgaaactagaattcatgttacatgtgcattagtctacgaaaagatgccaaccggtagaattcacaagggtgtatgctctacttggatgaaaaatgctgttccttacgaaaaatcagaaaagttgttcttaggtagaccaatcttcgtaagacaatcaaacttcaagttgccttctgattcaaaggttccaataatcatgataggtcctggtacaggtttagccccattcagaggtttcttgcaagaaagattggctttagttgaatctggtgtcgaattaggtccttcagttttgttctttggttgtagaaacagaagaatggatttcatctatgaagaagaattgcaaagattcgtcgaatctggtgcattggccgaattatctgtagctttttcaagagaaggtccaactaaggaatacgttcaacataagatgatggataaggcatccgacatatggaacatgatcagtcaaggtgcttatttgtacgtttgcggtgacgcaaagggtatggccagagatgtccatagatctttgcacacaattgctcaagaacaaggttccatggatagtaccaaagctgaaggtttcgtaaagaacttacaaacttccggtagatacttgagagatgtctggtga

SEQ ID NO: 92 - protein, 712 aa, Arabidopsis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDITLANGNGYTVFDAQHPYKANVAVKRELHTPESDRSCIHLEFDIAGSGLTMKLGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSEKLFLGRPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 93 - DNA, 1503 bp, Artificial Sequence
  Codon-optimized KAH
atggaagcctcttacctatacatttctattttgcttttactggcatcatacctgttcaccactcaacttagaaggaagagcgctaatctaccaccaaccgtgtttccatcaataccaatcattggacacttatacttactcaaaaagcctctttatagaactttagcaaaaattgccgctaagtacggaccaatactgcaattacaactcggctacagacgtgttctggtgatttcctcaccatcagcagcagaagagtgctttaccaataacgatgtaatcttcgcaaatagacctaagacattgtttggcaaaatagtgggtggaacatcccttggcagtttatcctacggcgatcaatggcgtaatctaaggagagtagcttctatcgaaatcctatcagttcataggttgaacgaatttcatgatatcagagtggatgagaacagattgttaattagaaaacttagaagttcatcttctcctgttactcttataacagtcttttatgctctaacattgaacgtcattatgagaatgatctctggcaaaagatatttcgacagtggggatagagaattggaggaggaaggtaagagatttcgagaaatcttagacgaaacgttgcttctagccggtgcttctaatgttggcgactacttaccaatattgaactggttgggagttaagtctcttgaaaagaaattgatcgctttgcagaaaaagagagatgactttttccagggtttgattgaacaggttagaaaatctcgtggtgctaaagtaggcaaaggtagaaaaacgatgatcgaactcttattatctttgcaagagtcagaacctgagtactatacagatgctatgataagatcttttgtcctaggtctgctggctgcaggtagtgatacttcagcgggcactatggaatgggccatgagcttactggtcaatcacccacatgtattgaagaaagctcaagctgaaatcgatagagttatcggtaataacagattgattgacgagtcagacattggaaatatcccttacatcgggtgtattatcaatgaaactctaagactctatccagcagggccattgttgttcccacatgaaagttctgccgactgcgttatttccggttacaatatacctagaggtacaatgttaatcgtaaaccaatgggcgattcatcacgatcctaaagtctgggatgatcctgaaacctttaaacctgaaagatttcaaggattagaaggaactagagatggtttcaaacttatgccattcggttctgggagaagaggatgtccaggtgaaggtttggcaataaggctgttagggatgacactaggctcagtgatccaatgttttgattgggagagagtaggagatgagatggttgacatgacagaaggtttgggtgtcacacttcctaaggccgttccattagttgccaaatgtaagccacgttccgaaatgactaatctcctatccgaactttaa

SEQ ID NO: 94 - protein, 500 aa, Stevia rebaudiana
MEASYLYISILLLLASYLFTTQLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLIRKLRSSSSPVTLITVFYALTLNVIMRMISGKRYFDSGDRELEEEGKRFREILDETLLLAGASNVGDYLPILNWLGVKSLEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVIGNNRLIDESDIGNIPYIGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

SEQ ID NO: 95 - DNA, 1572 bp, Rubus suavissimus
atggaagtaacagtagctagtagtgtagccctgagcctggtctttattagcatagtagtaagatgggcatggagtgtggtgaattgggtgtggtttaagccgaagaagctggaaagatttttgagggagcaaggccttaaaggcaattcctacaggtttttatatggagacatgaaggagaactctatcctgctcaaacaagcaagatccaaacccatgaacctctccacctcccatgacatagcacctcaagtcaccccttttgtcgaccaaaccgtgaaagcttacggtaagaactcttttaattgggttggccccataccaagggtgaacataatgaatccagaagatttgaaggacgtcttaacaaaaaatgttgactttgttaagccaatatcaaacccacttatcaagttgctagctacaggtattgcaatctatgaaggtgagaaatggactaaacacagaaggattatcaacccaacattccattcggagaggctaaagcgtatgttaccttcatttcaccaaagttgtaatgagatggtcaaggaatgggagagcttggtgtcaaaagagggttcatcatgtgagttggatgtctggccttttcttgaaaatatgtcggcagatgtgatctcgagaacagcatttggaactagctacaaaaaaggacagaaaatctttgaactcttgagagagcaagtaatatatgtaacgaaaggctttcaaagtttttacattccaggatggaggtttctcccaactaagatgaacaagaggatgaatgagattaacgaagaaataaaaggattaatcaggggtattataattgacagagagcaaatcattaaggcaggtgaagaaaccaacgatgacttattaggtgcacttatggagtcaaacttgaaggacattcgggaacatgggaaaaacaacaaaaatgttgggatgagtattgaagatgtaattcaggagtgtaagctgttttactttgctgggcaagaaaccacttcagtgttgctggcttggacaatggttttacttggtcaaaatcagaactggcaagatcgagcaagacaagaggttttgcaagtctttggaagcagcaagccagattttgatggtctagctcaccttaaagtcgtaaccatgattttgcttgaagttcttcgattatacccaccagtcattgaacttattcgaaccattcacaagaaaacacaacttgggaagctctcactaccagaaggagttgaagtccgcttaccaacactgctcattcaccatgacaaggaactgtggggtgatgatgcaaaccagttcaatccagagaggttttcggaaggagtttccaaagcaacaaagaaccgactctcattcttccccttcggagccggtccacgcatttgcattggacagaacttttctatgatggaagcaaagttggccttagcattgatcttgcaacacttcacctttgagctttctccatctcatgcacatgctccttcccatcgtataacccttcaaccacagtatggtgttcgtatcattttacatcgacgttag

SEQ ID NO: 96 - DNA, 1572 bp, Artificial Sequence
  Codon-optimized KAH
atggaagtcactgtcgcctcttctgtcgctttatccttagtcttcatttccattgtcgtcagatgggcttggtccgttgtcaactgggtttggttcaaaccaaagaagttggaaagattcttgagagagcaaggtttgaagggtaattcttatagattcttgtacggtgacatgaaggaaaattctattttgttgaagcaagccagatccaaaccaatgaacttgtctacctctcatgatattgctccacaagttactccattcgtcgatcaaactgttaaagcctacggtaagaactctttcaattgggttggtccaattcctagagttaacatcatgaacccagaagatttgaaggatgtcttgaccaagaacgttgacttcgttaagccaatttccaacccattgattaaattgttggctactggtattgccatttacgaaggtgaaaagtggactaagcatagaagaatcatcaaccctaccttccactctgaaagattgaagagaatgttaccatctttccatcaatcctgtaatgaaatggttaaggaatgggaatccttggtttctaaagaaggttcttcttgcgaattggatgtttggccattcttggaaaatatgtctgctgatgtcatttccagaaccgctttcggtacctcctacaagaagggtcaaaagattttcgaattgttgagagagcaagttatttacgttaccaagggtttccaatccttctacatcccaggttggagattcttgccaactaaaatgaacaagcgtatgaacgagatcaacgaagaaattaaaggtttgatcagaggtattattatcgacagagaacaaattattaaagctggtgaagaaaccaacgatgatttgttgggtgctttgatggagtccaacttgaaggatattagagaacatggtaagaacaacaagaatgttggtatgtctattgaagatgttattcaagaatgtaagttattctacttcgctggtcaagagaccacttctgttttgttagcctggactatggtcttgttaggtcaaaaccaaaattggcaagatagagctagacaagaagttttgcaagtcttcggttcttccaagccagactttgatggtttggcccacttgaaggttgttactatgattttgttagaagttttgagattgtacccaccagtcattgagttaatcagaaccattcataaaaagactcaattgggtaaattatctttgccagaaggtgttgaagtcagattaccaaccttgttgattcaccacgataaggaattatggggtgacgacgctaatcaatttaatccagaaagattttccgaaggtgtttccaaggctaccaaaaaccgtttgtccttcttcccatttggtgctggtccacgtatttgtatcggtcaaaacttttccatgatggaagccaagttggctttggctttaatcttgcaacacttcactttcgaattgtctccatcccatgcccacgctccttctcatagaatcactttacaaccacaatacggtgtcagaatcatcttacacagaagataa

SEQ ID NO: 97 - protein, 523 aa, Rubus suavissimus
MEVTVASSVALSLVFISIVVRWAWSVVNWVWFKPKKLERFLREQGLKGNSYRFLYGDMKENSILLKQARSKPMNLSTSHDIAPQVTPFVDQTVKAYGKNSFNWVGPIPRVNIMNPEDLKDVLTKNVDFVKPISNPLIKLLATGIAIYEGEKWTKHRRIINPTFHSERLKRMLPSFHQSCNEMVKEWESLVSKEGSSCELDVWPFLENMSADVISRTAFGTSYKKGQKIFELLREQVIYVTKGFQSFYIPGWRFLPTKMNKRMNEINEEIKGLIRGIIIDREQIIKAGEETNDDLLGALMESNLKDIREHGKNNKNVGMSIEDVIQECKLFYFAGQETTSVLLAWTMVLLGQNQNWQDRARQEVLQVFGSSKPDFDGLAHLKVVTMILLEVLRLYPPVIELIRTIHKKTQLGKLSLPEGVEVRLPTLLIHHDKELWGDDANQFNPERFSEGVSKATKNRLSFFPFGAGPRICIGQNFSMMEAKLALALILQHFTFELSPSHAHAPSHRITLQPQYGVRIILHRR

SEQ ID NO: 98 - DNA, 1566 bp, Prunus avium
atggaagcatcaagggctagttgtgttgcgctatgtgttgtttgggtgagcatagtaattacattggcatggagggtgctgaattgggtgtggttgaggccaaagaaactagaaagatgcttgagggagcaaggccttacaggcaattcttacaggcttttgtttggagacaccaaggatctctcgaagatgctggaacaaacacaatccaaacccatcaaactctccacctcccatgatatagcgccacgagtcaccccatttttccatcgaactgtgaactctaatggcaagaattcttttgtttggatgggccctataccaagagtgcacatcatgaatccagaagatttgaaagatgccttcaacagacatgatgattttcataagacagtaaaaaatcctatcatgaagtctccaccaccgggcattgtaggcattgaaggtgagcaatgggctaaacacagaaagattatcaacccagcattccatttagagaagctaaagggtatggtaccaatattttaccaaagttgtagcgagatgattaacaaatgggagagcttggtgtccaaagagagttcatgtgagttggatgtgtggccttatcttgaaaattttaccagcgatgtgatttcccgagctgcatttggaagtagctatgaagagggaaggaaaatatttcaactactaagagaggaagcaaaagtttattcggtagctctacgaagtgtttacattccaggatggaggtttctaccaaccaagcagaacaagaagacgaaggaaattcacaatgaaattaaaggcttacttaagggcattataaataaaagggaagaggcgatgaaggcaggggaagccactaaagatgacttactaggaatacttatggagtccaacttcagggaaattcaggaacatgggaacaacaaaaatgctggaatgagtattgaagatgtaattggagagtgtaagttgttttactttgctgggcaagagaccacttcggtgttgcttgtttggacaatgattttactaagccaaaatcaggattggcaagctcgtgcaagagaagaggtcttgaaagtctttggaagcaacatcccaacctatgaagagctaagtcacctaaaagttgtgaccatgattttacttgaagttcttcgattatacccatcagtcgttgcgcttcctcgaaccactcacaagaaaacacagcttggaaaattatcattaccagctggagtggaagtctccttgcccatactgcttgttcaccatgacaaagagttgtggggtgaggatgcaaatgagttcaagccagagaggttttcagagggagtttcaaaggcaacaaagaacaaatttacatacttacctttcggagggggtccaaggatttgcattggacaaaactttgccatggtggaagctaaattggccttggccctgattttacaacactttgcctttgagctttctccatcctatgctcatgctccttctgcagttataacccttcaacctcaatttggtgctcatatcattttgcataaacgttga

SEQ ID NO: 99 - DNA, 1567 bp, Artificial Sequence
  Codon-optimized KAH
atggaagcttctagagcatcttgtgttgctttgtgtgttgtttgggtttccatcgttattactttggcttggagagttttgaattgggtctggttaagaccaaaaaagttggaaagatgcttgagagaacaaggtttgactggtaactcttacagattgttgttcggtgataccaaggacttgtctaagatgttggaacaaactcaatccaagcctatcaagttgtctacctctcatgatattgctccaagagttactccattcttccatagaactgttaactccaacggtaagaactcttttgtttggatgggtccaattccaagagtccatattatgaaccctgaagatttgaaggacgctttcaacagacatgatgatttccataagaccgtcaagaacccaattatgaagtctccaccaccaggtatagttggtattgaaggtgaacaatgggccaaacatagaaagattattaacccagccttccacttggaaaagttgaaaggtatggttccaatcttctaccaatcctgctctgaaatgattaacaagtgggaatccttggtttccaaagaatcttcctgtgaattggatgtctggccatatttggaaaacttcacctccgatgttatttccagagctgcttttggttcttcttacgaagaaggtagaaagatcttccaattattgagagaagaagccaaggtttactccgttgctttgagatctgtttacattccaggttggagattcttgccaactaagcaaaacaaaaagaccaaagaaatccacaacgaaatcaagggtttgttgaagggtatcatcaacaagagagaagaagctatgaaggctggtgaagctacaaaagatgatttgttgggtatcttgatggaatccaacttcagagaaatccaagaacacggtaacaacaagaatgccggtatgtctattgaagatgttatcggtgaatgcaagttgttctactttgctggtcaagaaactacctccgttttgttggtttggaccatgattttgttgtcccaaaatcaagattggcaagctagagctagagaagaagtcttgaaagttttcggttctaacatcccaacctacgaagaattgtctcacttgaaggttgtcactatgatcttgttggaagtattgagattatacccatccgttgttgcattgccaagaactactcataagaaaactcaattgggtaaattgtccttgccagctggtgttgaagtttctttgccaattttgttagtccaccacgacaaagaattgtggggtgaagatgctaatgaattcaagccagaaagattctccgaaggtgtttctaaagctaccaagaacaagttcacttacttgccatttggtggtggtccaagaatatgtattggtcaaaatttcgctatggtcgaagctaaattggctttggctttgatcttgcaacatttcgctttcgaattgtcaccatcttatgctcatgctccatctgctgttattacattgcaaccacaatttggtgcccatatcatcttgcataagagataac

SEQ ID NO: 100 - protein, 521 aa, Prunus avium
MEASRASCVALCVVWVSIVITLAWRVLNWVWLRPKKLERCLREQGLTGNSYRLLFGDTKDLSKMLEQTQSKPIKLSTSHDIAPRVTPFFHRTVNSNGKNSFVWMGPIPRVHIMNPEDLKDAFNRHDDFHKTVKNPIMKSPPPGIVGIEGEQWAKHRKIINPAFHLEKLKGMVPIFYQSCSEMINKWESLVSKESSCELDVWPYLENFTSDVISRAAFGSSYEEGRKIFQLLREEAKVYSVALRSVYIPGWRFLPTKQNKKTKEIHNEIKGLLKGIINKREEAMKAGEATKDDLLGILMESNFREIQEHGNNKNAGMSIEDVIGECKLFYFAGQETTSVLLVWTMILLSQNQDWQARAREEVLKVFGSNIPTYEELSHLKVVTMILLEVLRLYPSVVALPRTTHKKTQLGKLSLPAGVEVSLPILLVHHDKELWGEDANEFKPERFSEGVSKATKNKFTYLPFGGGPRICIGQNFAMVEAKLALALILQHFAFELSPSYAHAPSAVITLQPQFGAHIILHKR

SEQ ID NO: 101 - protein, 517 aa, Prunus mume
ASWVAVLSVVWVSMVIAWAWRVLNWVWLRPKKLEKCLREQGLAGNSYRLLFGDTKDLSKMLEQTQSKPIKLSTSHDIAPHVTPFFHQTVNSYGKNSFVWMGPIPRVHIMNPEDLKDTFNRHDDFHKVVKNPIMKSLPQGIVGIEGEQWAKHRKIINPAFHLEKLKGMVPIFYRSCSEMINKWESLVSKESSCELDVWPYLENFTSDVISRAAFGSSYEEGRKIFQLLREEAKIYTVAMRSVYIPGWRFLPTKQNKKAKEIHNEIKGLLKGIINKREEAMKAGEATKDDLLGILMESNFREIQEHGNNKNAGMSIEDVIGECKLFYFAGQETTSVLLVWTMVLLSQNQDWQARAREEVLQVFGSNIPTYEELSQLKVVTMILLEVLRLYPSVVALPRTTHKKTQLGKLSLPAGVEVSLPILLVHHDKELWGEDANEFKPERFSEGVSKATKNQFTYFPFGGGPRICIGQNFAMMEAKLALSLILRHFALELSPLYAHAPSVTITLQPQYGAHIILHKR

SEQ ID NO: 102 - protein, 521 aa, Prunus mume
MEASRPSCVALSVVLVSIVIAWAWRVLNWVWLRPNKLERCLREQGLTGNSYRLLFGDTKEISMMVEQAQSKPIKLSTTHDIAPRVIPFSHQIVYTYGRNSFVWMGPTPRVTIMNPEDLKDAFNKSDEFQRAISNPIVKSISQGLSSLEGEKWAKHRKIINPAFHLEKLKGMLPTFYQSCSEMINKWESLVFKEGSREMDVWPYLENLTSDVISRAAFGSSYEEGRKIFQLLREEAKFYTIAARSVYIPGWRFLPTKQNKRMKEIHKEVRGLLKGIINKREDAIKAGEAAKGNLLGILMESNFREIQEHGNNKNAGMSIEDVIGECKLFYFAGQETTSVLLVWTLVLLSQNQDWQARAREEVLQVFGTNIPTYDQLSHLKVVTMILLEVLRLYPAVVELPRTTYKKTQLGKFLLPAGVEVSLHIMLAHHDKELWGEDAKEFKPERFSEGVSKATKNQFTYFPFGAGPRICIGQNFAMLEAKLALSLILQHFTFELSPSYAHAPSVTITLHPQFGAHFILHKR

SEQ ID NO: 103 - protein, 514 aa, Prunus mume
CVALSVVLVSIVIAWAWRVLNWVWLRPNKLERCLREQGLTGNSYRLLFGDTKEISMMVEQAQSKPIKLSTTHDIAPRVIPFSHQIVYTYGRNSFVWMGPTPRVTIMNPEDLKDAFNKSDEFQRAISNPIVKSISQGLSSLEGEKWAKHRKIINPAFHLEKLKGMLPTFYQSCSEMINKWESLVFKEGSREMDVWPYLENLTSDVISRAAFGSSYEEGRKIFQLLREEAKFYTIAARSVYIPGWRFLPTKQNKRMKEIHKEVRGLLKGIINKREDAIKAGEAAKGNLLGILMESNFREIQEHGNNKNAGMSIEDVIGECKLFYFAGQETTSVLLVWTLVLLSQNQDWQARAREEVLQVFGTNIPTYDQLSHLKVVTMILLEVLRLYPAVVELPRTTYKKTQLGKFLLPAGVEVSLHIMLAHHDKELWGEDAKEFKPERFSEGVSKATKNQFTYFPFGAGPRICIGQNFAMLEAKLALSLILQHFTFELSPSYAHAPSVTITLHPQFGAHFILHKR

SEQ ID NO: 104 - protein, 418 aa, Prunus persica
MGPIPRVHIMNPEDLKDTFNRHDDFHKVVKNPIMKSLPQGIVGIEGDQWAKHRKIINPAFHLEKLKGMVPIFYQSCSEMINIWKSLVSKESSCELDVWPYLENFTSDVISRAAFGSSYEEGRKIFQLLREEAKVYTVAVRSVYIPGWRFLPTKQNKKTKEIHNEIKGLLKGIINKREEAMKAGEATKDDLLGILMESNFREIQEHGNNKNAGMSIEDVIGECKLFYFAGQETTSVLLVWTMVLLSQNQDWQARAREEVLQVFGSNIPTYEELSHLKVVTMILLEVLRLYPSVVALPRTTHKKTQLGKLSLPAGVEVSLPILLVHHDKELWGEDANEFKPERFSEGVSKATKNQFTYFPFGGGPRICIGQNFAMMEAKLALSLILQHFTFELSPQYSHAPSVTITLQPQYGAHLILHKR

SEQ ID NO: 105 - DNA, 1578 bp, Artificial Sequence
  Codon-optimized KAH
atgggtttgttcccattagaggattcctacgcgctggtctttgaaggactagcaataacactggctttgtactatctactgtctttcatctacaaaacatctaaaaagacatgtacacctcctaaagcatctggtgaaatcattccaattacaggaatcatattgaatctgctatctggctcaagtggtctacctattatcttagcacttgcctctttagcagacagatgtggtcctattttcaccattaggctgggtattaggagagtgctagtagtatcaaattgggaaatcgctaaggagattttcactacccacgatttgatagtttctaatagaccaaaatacttagccgctaagattcttggtttcaattatgtttcattctctttcgctccatacggcccatattgggtcggaatcagaaagattattgctacaaaactaatgtcttcttccagacttcagaagttgcaatttgtaagagtttttgaactagaaaactctatgaaatctatcagagaatcatggaaggagaaaaaggatgaagagggaaaggtattagttgagatgaaaaagtggttctgggaactgaatatgaacatagtgttaaggacagttgctggtaaacaatacactggtacagttgatgatgccgatgcaaagcgtatctccgagttattcagagaatggtttcactacactggcagatttgtcgttggagacgcttttccttttctaggttggttggacctgggcggatacaaaaagacaatggaattagttgctagtagattggactcaatggtcagtaaatggttagatgagcatcgtaaaaagcaagctaacgatgacaaaaaggaggatatggatttcatggatatcatgatctccatgacagaagcaaattcaccacttgaaggatacggcactgatactattatcaagaccacatgtatgactttgattgtttcaggagttgatacaacctcaatcgtacttacttgggccttatcacttttgttaaacaacagagatactttgaaaaaggcacaagaggaattagatatgtgcgtaggtaaaggaagacaagtcaacgagtctgatcttgttaacttgatatacttggaagcagtgcttaaagaggctttaagactttacccagcagcgttcttaggcggaccaagagcattcttggaagattgtactgttgctggttatagaattccaaagggcacctgcttgttgattaacatgtggaaactgcatagagatccaaacatttggagtgatccttgcgaattcaagccagaaagatttttgacacctaatcaaaaggatgttgatgtgatcggtatggatttcgaattgataccatttggtgccggcagaagatattgtccaggtactagattggctttacagatgttgcatatcgtattagcgacattgctgcaaaacttcgaaatgtcaacaccaaacgatgcgccagtcgatatgactgcttctgttggcatgacaaatgccaaagcatcacctttagaagtcttgctatcacctcgtgttaaatggtcctaa

SEQ ID NO: 106 - protein, 522 aa, Stevia rebaudiana
MGLFPLEDSYALVFEGLAITLALYYLLSFIYKTSKKTCTPPKASGEHPITGHLNLLSGSSGLPHLALASLADRCGPIFTIRLGIRRVLVVSNWEIAKEIFTTHDLIVSNRPKYLAAKILGFNYVSFSFAPYGPYWVGIRKIIATKLMSSSRLQKLQFVRVFELENSMKSIRESWKEKKDEEGKVLVEMKKWFWELNMNIVLRTVAGKQYTGTVDDADAKRISELFREWFHYTGRFVVGDAFPFLGWLDLGGYKKTMELVASRLDSMVSKWLDEHRKKQANDDKKEDMDFMDIMISMTEANSPLEGYGTDTIIKTTCMTLIVSGVDTTSIVLTWALSLLLNNRDTLKKAQEELDMCVGKGRQVNESDLVNLIYLEAVLKEALRLYPAAFLGGPRAFLEDCTVAGYRIPKGTCLLINMWKLHRDPNIWSDPCEFKPERFLTPNQKDVDVIGMDFELIPFGAGRRYCPGTRLALQMLHIVLATLLQNFEMSTPNDAPVDMTASVGMTNAKASPLEVLLSPRVKWS

SEQ ID NO: 107 - DNA, 1431 bp, Artificial Sequence
  Codon-optimized KAH
atgatacaagttttaactccaattctactcttcctcatcttcttcgttttctggaaagtctacaaacatcaaaagactaaaatcaatctaccaccaggttccttcggctggccatttttgggtgaaaccttagccttacttagagcaggctgggattctgagccagaaagattcgtaagagagcgtatcaaaaagcatggatctccacttgttttcaagacatcactatttggagacagattcgctgttctttgcggtccagctggtaataagtttttgttctgcaacgaaaacaaattagtggcatcttggtggccagtccctgtaaggaagttgttcggtaaaagtttactcacaataagaggagatgaagcaaaatggatgagaaaaatgctattgtcttacttgggtccagatgcatttgccacacattatgccgttactatggatgttgtaacacgtagacatattgatgtccattggaggggcaaggaggaagttaatgtatttcaaacagttaagttgtacgcattcgaattagcttgtagattattcatgaacctagatgacccaaaccacatcgcgaaactcggtagtcttttcaacattttcctcaaagggatcatcgagcttcctatagacgttcctggaactagattttactccagtaaaaaggccgcagctgccattagaattgaattgaaaaagctcattaaagctagaaaactcgaattgaaggagggtaaggcgtcttcttcacaggacttgctttctcatctattaacatcacctgatgagaatgggatgttcttgacagaagaggaaatagtcgataacattctacttttgttattcgctggtcacgatacctctgcactatcaataacacttttgatgaaaaccttaggtgaacacagtgatgtgtacgacaaggttttgaaggaacaattagaaatttccaaaacaaaggaggcttgggaatcactaaagtgggaagatatccagaagatgaagtactcatggtcagtaatctgtgaagtcatgagattgaatcctcctgtcatagggacatacagagaggcgttggttgatatcgactatgctggttacactatcccaaaaggatggaagttgcattggtcagctgtttctactcaaagagacgaagccaatttcgaagatgtaactagattcgatccatccagatttgaaggggcaggccctactccattcacatttgtgcctttcggtggaggtcctagaatgtgtttaggcaaagagtttgccaggttagaagtgttagcatttctccacaacattgttaccaactttaagtgggatcttctaatccctgatgagaagatcgaatatgatccaatggctactccagctaagggcttgccaattagacttcatccacaccaagtctaa

SEQ ID NO: 108 - protein, 476 aa, Stevia rebaudiana
MIQVLTPILLFLIFFVFWKVYKHQKTKINLPPGSFGWPFLGETLALLRAGWDSEPERFVRERIKKHGSPLVFKTSLFGDRFAVLCGPAGNKFLFCNENKLVASWWPVPVRKLFGKSLLTIRGDEAKWMRKMLLSYLGPDAFATHYAVTMDVVTRRHIDVHWRGKEEVNVFQTVKLYAFELACRLFMNLDDPNHIAKLGSLFNIFLKGIIELPIDVPGTRFYSSKKAAAAIRIELKKLIKARKLELKEGKASSSQDLLSHLLTSPDENGMFLTEEEIVDNILLLLFAGHDTSALSITLLMKTLGEHSDVYDKVLKEQLEISKTKEAWESLKWEDIQKMKYSWSVICEVMRLNPPVIGTYREALVDIDYAGYTIPKGWKLHWSAVSTQRDEANFEDVTRFDPSRFEGAGPTPFTFVPFGGGPRMCLGKEFARLEVLAFLHNIVTNFKWDLLIPDEKIEYDPMATPAKGLPIRLHPHQV

SEQ ID NO: 109 - DNA, 1578 bp, Artificial Sequence
  Codon-optimized KAH
atggagtctttagtggttcatacagtaaatgctatctggtgtattgtaatcgtcgggattttctcagttggttatcacgtttacggtagagctgtggtcgaacaatggagaatgagaagatcactgaagctacaaggtgttaaaggcccaccaccatccatcttcaatggtaacgtctcagaaatgcaacgtatccaatccgaagctaaacactgctctggcgataacattatctcacatgattattcttcttcattattcccacacttcgatcactggagaaaacagtacggcagaatctacacatactctactggattaaagcaacacttgtacatcaatcatccagaaatggtgaaggagctatctcagactaacacattgaacttgggtagaatcacccatataaccaaaagattgaatcctatcttaggtaacggaatcataacctctaatggtcctcattgggcccatcagcgtagaattatcgcctacgagtttactcatgataagatcaagggtatggttggtttgatggttgagtctgctatgcctatgttgaataagtgggaggagatggtaaagagaggcggagaaatgggatgcgacataagagttgatgaggacttgaaagatgtttcagcagatgtgattgcaaaagcctgtttcggatcctcattttctaaaggtaaggctattttctctatgataagagatttgcttacagctatcacaaagagaagtgttctattcagattcaacggattcactgatatggtctttgggagtaaaaagcatggtgacgttgatatagacgctttagaaatggaattggaatcatccatttgggaaactgtcaaggaacgtgaaatagaatgtaaagatactcacaaaaaggatctgatgcaattgattttggaaggggcaatgcgttcatgtgacggtaacctttgggataaatcagcatatagaagatttgttgtagataattgtaaatctatctacttcgcagggcatgatagtacagctgtctcagtgtcatggtgtttgatgttactggccctaaacccatcatggcaagttaagatccgtgatgaaattctgtcttcttgcaaaaatggtattccagatgccgaaagtatcccaaaccttaaaacagtgactatggttattcaagagacaatgagattataccctccagcaccaatcgtcgggagagaagcctctaaagatatcagattgggcgatctagttgttcctaaaggcgtctgtatatggacactaataccagctttacacagagatcctgagatttggggaccagatgcaaacgatttcaaaccagaaagattttctgaaggaatttcaaaggcttgtaagtatcctcaaagttacattccatttggtctgggtcctagaacatgcgttggtaaaaactttggcatgatggaagtaaaggttcttgtttccctgattgtctccaagttctctttcactctatctcctacctaccaacatagtcctagtcacaaacttttagtagaaccacaacatggggtggtaattagagtggtttaa

SEQ ID NO: 110 - protein, 525 aa, Arabidopsis thaliana
MESLVVHTVNAIWCIVIVGIFSVGYHVYGRAVVEQWRMRRSLKLQGVKGPPPSIFNGNVSEMQRIQSEAKHCSGDNIISHDYSSSLFPHFDHWRKQYGRIYTYSTGLKQHLYINHPEMVKELSQTNTLNLGRITHITKRLNPILGNGIITSNGPHWAHQRRIIAYEFTHDKIKGMVGLMVESAMPMLNKWEEMVKRGGEMGCDIRVDEDLKDVSADVIAKACFGSSFSKGKAIFSMIRDLLTAITKRSVLFRFNGFTDMVFGSKKHGDVDIDALEMELESSIWETVKEREIECKDTHKKDLMQLILEGAMRSCDGNLWDKSAYRRFVVDNCKSIYFAGHDSTAVSVSWCLMLLALNPSWQVKIRDEILSSCKNGIPDAESIPNLKTVTMVIQETMRLYPPAPIVGREASKDIRLGDLVVPKGVCIWTLIPALHRDPEIWGPDANDFKPERFSEGISKACKYPQSYIPFGLGPRTCVGKNFGMMEVKVLVSLIVSKFSFTLSPTYQHSPSHKLLVEPQHGVVIRVV

SEQ ID NO: 111 - DNA, 1590 bp, Artificial Sequence
  Codon-optimized KAH
atgtacttcctactacaatacctcaacatcacaaccgttggtgtctttgccacattgtttctctcttattgtttacttctctggagaagtagagcgggtaacaaaaagattgccccagaagctgccgctgcatggcctattatcggccacctccacttacttgcaggtggatcccatcaactaccacatattacattgggtaacatggcagataagtacggtcctgtattcacaatcagaataggcttgcatagagctgtagttgtctcatcttgggaaatggcaaaggaatgttcaacagctaatgatcaagtgtcttcttcaagacctgaactattagcttctaagttgttgggttataactacgccatgtttggtttttcaccatacggttcatactggagagaaatgagaaagatcatctctctcgaattactatctaattccagattggaactattgaaagatgttagagcctcagaagttgtcacatctattaaggaactatacaaattgtgggcggaaaagaagaatgagtcaggattggtttctgtcgagatgaaacaatggttcggagatttgactttaaacgtgatcttgagaatggtggctggtaaaagatacttctccgcgagtgacgcttcagaaaacaaacaggcccagcgttgtagaagagtcttcagagaattcttccatctctccggcttgtttgtggttgctgatgctataccttttcttggatggctcgattggggaagacacgagaagaccttgaaaaagaccgccatagaaatggattccatcgcccaggagtggcttgaggaacatagacgtagaaaagattctggagatgataattctacccaagatttcatggacgttatgcaatctgtgctagatggcaaaaatctaggcggatacgatgctgatacgattaacaaggctacatgcttaactcttatatcaggtggcagtgatactactgtagtttctttgacatgggctcttagtcttgtgttaaacaatagagatactttgaaaaaggcacaggaagagttagacatccaagtcggtaaggaaagattggttaacgagcaagacatcagtaagttagtttacttgcaagcaatagtaaaagagacactcagactttatccaccaggtcctttgggtggtttgagacaattcactgaagattgtacactaggtggctatcacgtttcaaaaggaactagattaatcatgaacttatccaagattcaaaaagatccacgtatttggtctgatcctactgaattccaaccagagagattccttacgactcataaagatgtcgatccacgtggtaaacactttgaattcattccattcggtgcaggaagacgtgcatgtcctggtatcacattcggattacaagtactacatctaacattggcatctttcttgcatgcgtttgaattttcaacaccatcaaatgagcaggttaacatgagagaatcattaggtcttacgaatatgaaatctaccccattagaagttttgatttctccaagactatcccttaattgcttcaaccttatgaaaatttga

SEQ ID NO: 112 - protein, 526 aa, Vitis vinifera
MYFLLQYLNITTVGVFATLFLSYCLLLWRSRAGNKKIAPEAAAAWPIIGHLHLLAGGSHQLPHITLGNMADKYGPVFTIRIGLHRAVVVSSWEMAKECSTANDQVSSSRPELLASKLLGYNYAMFGFSPYGSYWREMRKIISLELLSNSRLELLKDVRASEVVTSIKELYKLWAEKKNESGLVSVEMKQWFGDLTLNVILRMVAGKRYFSASDASENKQAQRCRRVFREFFHLSGLFVVADAIPFLGWLDWGRHEKTLKKTAIEMDSIAQEWLEEHRRRKDSGDDNSTQDFMDVMQSVLDGKNLGGYDADTINKATCLTLISGGSDTTVVSLTWALSLVLNNRDTLKKAQEELDIQVGKERLVNEQDISKLVYLQAIVKETLRLYPPGPLGGLRQFTEDCTLGGYHVSKGTRLIMNLSKIQKDPRIWSDPTEFQPERFLTTHKDVDPRGKHFEFIPFGAGRRACPGITFGLQVLHLTLASFLHAFEFSTPSNEQVNMRESLGLTNMKSTPLEVLISPRLSSCSLYN

SEQ ID NO: 113 - DNA, 1440 bp, Artificial Sequence
  Codon-optimized KAH
atggaacctaacttttacttgtcattactattgttgttcgtgaccttcatttctttaagtctgtttttcatcttttacaaacaaaagtccccattgaatttgccaccagggaaaatgggttaccctatcataggtgaaagtttagaattcctatccacaggctggaagggacatcctgaaaagttcatatttgatagaatgcgtaagtacagtagtgagttattcaagacttctattgtaggcgaatccacagttgtttgctgtggggcagctagtaacaaattcctattctctaacgaaaacaaactggtaactgcctggtggccagattctgttaacaaaatcttcccaacaacttcactggattctaatttgaaggaggaatctataaagatgagaaagttgctgccacagttcttcaaaccagaagcacttcaaagatacgtcggcgttatggatgtaatcgcacaaagacattttgtcactcactgggacaacaaaaatgagatcacagtttatccacttgctaaaagatacactttcttgcttgcgtgtagactgttcatgtctgttgaggatgaaaatcatgtggcgaaattctcagacccattccaactaatcgctgcaggcatcatttcacttcctatcgatcttcctggtactccattcaacaaggccataaaggcttcaaatttcattagaaaagagctgataaagattatcaaacaaagacgtgttgatctggcagagggtacagcatctccaacccaggatatcttgtcacatatgctattaacatctgatgaaaacggtaaatctatgaacgagttgaacattgccgacaagattcttggactattgataggaggccacgatacagcttcagtagcttgcacatttctagtgaagtacttaggagaattaccacatatctacgataaagtctaccaagagcaaatggaaattgccaagtccaaacctgctggggaattgttgaattgggatgacttgaaaaagatgaagtattcatggaatgtggcatgtgaggtaatgagattgtcaccacctttacaaggtggttttagagaggctataactgactttatgtttaacggtttctctattccaaaagggtggaagttatactggtccgccaactctacacacaaaaatgcagaatgtttcccaatgcctgagaaattcgatcctaccagatttgaaggtaatggtccagcgccttatacatttgtaccattcggtggaggccctagaatgtgtcctggaaaggaatacgctagattagaaatcttggttttcatgcataatctggtcaaacgttttaagtgggaaaaggttattccagacgaaaagattattgtcgatccattcccaatcccagctaaagatcttccaatccgtttgtatcctcacaaagcttaa

SEQ ID NO: 114 - protein, 479 aa, Medicago truncatula
MEPNFYLSLLLLFVTFISLSLFFIFYKQKSPLNLPPGKMGYPIIGESLEFLSTGWKGHPEKFIFDRMRKYSSELFKTSIVGESTVVCCGAASNKFLFSNENKLVTAWWPDSVNKIFPTTSLDSNLKEESIKMRKLLPQFFKPEALQRYVGVMDVIAQRHFVTHWDNKNEITVYPLAKRYTFLLACRLFMSVEDENHVAKFSDPFQLIAAGIISLPIDLPGTPFNKAIKASNFIRKELIKIIKQRRVDLAEGTASPTQDILSHMLLTSDENGKSMNELNIADKILGLLIGGHDTASVACTFLVKYLGELPHIYDKVYQEQMEIAKSKPAGELLNWDDLKKMKYSWNVACEVMRLSPPLQGGFREAITDFMFNGFSIPKGWKLYWSANSTHKNAECFPMPEKFDPTRFEGNGPAPYTFVPFGGGPRMCPGKEYARLEILVFMHNLVKRFKWEKVIPDEKIIVDPFPIPAKDLPIRLYPHKA

SEQ ID NO: 115 - DNA, 1116 bp, Artificial Sequence
  Codon-optimized GGPPS
atggcctctgttactttgggttcctggatcgtcgtccaccaccataaccatcaccatccatcatctatcctaactaaatctcgttcaagatcctgtcctattacactaaccaaaccaatctcttttcgttcaaagagaacagtttcctctagtagttctatcgtgtcctctagtgtcgtcactaaggaagacaatctgagacagtctgaaccttcttcctttgatttcatgtcatatatcattactaaggcagaactagtgaataaggctcttgattcagcagttccattaagagagccattgaaaatccatgaagcaatgagatactctcttctagctggcgggaagagagtcagacctgtactctgcatagcagcgtgcgaattagttggtggcgaggaatcaaccgctatgcctgccgcttgtgctgtagaaatgattcatacaatgtcactgatacacgatgatttgccatgtatggataacgatgatctgagaaggggtaagccaactaaccataaggttttcggcgaagatgttgccgtcttagctggtgatgctttgttatctttcgcgttcgaacatttggcatccgcaacatcaagtgatgttgtgtcaccagtaagagtagttagagcagttggagaactggctaaagctattggaactgagggtttagttgcaggtcaagtcgtcgatatctcttccgaaggtcttgatttgaatgatgtaggtcttgaacatctcgaattcatccatcttcacaagacagctgcacttttagaagccagtgcggttctcggcgcaattgttggcggagggagtgatgacgaaattgagagattgaggaagtttgctagatgtataggattactgttccaagtagtagacgatatactagatgtgacaaagtcttccaaagagttgggaaaaacagctggtaaagatttgattgccgacaaattgacctaccctaagattatggggctagaaaaatcaagagaatttgccgagaaactcaatagagaggcgcgtgatcaactgttgggtttcgattctgataaagttgcaccactcttagccttagccaactacatcgcttacagacaaaactaa

SEQ ID NO: 116 - protein, 371 aa, Arabidopsis thaliana
MASVTLGSWIVVHHHNHHHPSSILTKSRSRSCPITLTKPISFRSKRTVSSSSSIVSSSVVTKEDNLRQSEPSSFDFMSYIITKAELVNKALDSAVPLREPLKIHEAMRYSLLAGGKRVRPVLCIAACELVGGEESTAMPAACAVEMIHTMSLIHDDLPCMDNDDLRRGKPTNHKVFGEDVAVLAGDALLSFAFEHLASATSSDVVSPVRVVRAVGELAKAIGTEGLVAGQVVDISSEGLDLNDVGLEHLEFIHLHKTAALLEASAVLGAIVGGGSDDEIERLRKFARCIGLLFQVVDDILDVTKSSKELGKTAGKDLIADKLTYPKIMGLEKSREFAEKLNREARDQLLGFDSDKVAPLLALANYIAYRQN

SEQ ID NO: 117 - protein, 511 aa, Rubus suavissimus
MATLLEHFQAMPFAIPIALAALSWLFLFYIKVSFFSNKSAQAKLPPVPVVPGLPVIGNLLQLKEKKPYQTFTRWAEEYGPIYSIRTGASTMVVLNTTQVAKEAMVTRYLSISTRKLSNALKILTADKCMVAISDYNDFHKMIKRYILSNVLGPSAQKRHRSNRDTLRANVCSRLHSQVKNSPREAVNFRRVFEWELFGIALKQAFGKDIEKPIYVEELGTTLSRDEIFKVLVLDIMEGAIEVDWRDFFPYLRWIPNTRMETKIQRLYFRRKAVMTALINEQKKRIASGEEINCYIDFLLKEGKTLTMDQISMLLWETVIETADTTMVTTEWAMYEVAKDSKRQDRLYQEIQKVCGSEMVTEEYLSQLPYLNAVFHETLRKHSPAALVPLRYAHEDTQLGGYYIPAGTEIAINIYGCNMDKHQWESPEEWKPERFLDPKFDPMDLYKTMAFGAGKRVCAGSLQAMLIACPTIGRLVQEFEWKLRDGEEENVDTVGLTTHKRYPMHAILKPRS

SEQ ID NO: 118 - DNA, 1710 bp, Saccharomyces cerevisiae
atgtcatttcaaattgaaacggttcccaccaaaccatatgaagaccaaaagcctggtacctctggtttgcgtaagaagacaaaggtgtttaaagacgaacctaactacacagaaaatttcattcaatcgatcatggaagctattccagagggttctaaaggtgccactcttgttgtcggtggtgatgggcgttactacaatgatgtcattcttcataagattgccgctatcggtgctgccaacggtattaaaaagttagttattggccagcatggtcttctgtctacgccagccgcttctcacatcatgagaacctacgaggaaaaatgtactggtggtattatcttaaccgcctcacataatccaggtggtccagaaaatgacatgggtattaagtataacttatccaatgggggtcctgctcctgaatccgtcacaaatgctatttgggagatttccaaaaagcttaccagctataagattatcaaagacttcccagaactagacttgggtacgataggcaagaacaagaaatacggtccattactcgttgacattatcgatattacaaaagattatgtcaacttcttgaaggaaatcttcgatttcgacttaatcaagaaattcatcgataatcaacgttctactaagaattggaagttactgtttgacagtatgaacggtgtaactggaccatacggtaaggctattttcgttgatgaatttggtttaccggcggatgaggttttacaaaactggcatccttctccggattttggtggtatgcatccagatccaaacttaacttatgccagttcgttagtgaaaagagtagatcgtgaaaagattgagtttggtgctgcatccgatggtgatggtgatagaaatatgatttacggttacggcccatctttcgtttctccaggtgactccgtcgcaattattgccgaatatgcagctgaaatcccatatttcgccaagcaaggtatatatggtctggcccgttcattccctacctcaggagccatagaccgtgttgccaaggcccatggtctaaactgttatgaggtcccaactggctggaaatttttctgtgctttgttcgacgctaaaaaattatctatttgtggtgaagaatcgtttggtactggttccaaccacgtaagggaaaaggacggtgtttgggccattatggcgtggttgaacatcttggccatttacaacaagcatcatccggagaacgaagcttctattaagacgatacagaatgaattctgggcaaagtacggccgtactttcttcactcgttatgattttgaaaaagttgaaacagaaaaagctaacaagattgtcgatcaattgagagcatatgttaccaaatcgggtgttgttaattccgccttcccagccgatgagtctcttaaggtcaccgattgtggtgatttttcatacacagatttggacggttctgtttctgaccatcaaggtttatatgtcaagctttccaatggtgcaagattcgttctaagattgtcaggtacaggttcttcaggtgctaccattagattgtacattgaaaaatactgcgatgataaatcacaataccaaaagacagctgaagaatacttgaagccaattattaactcggtcatcaagttcttgaactttaaacaagttttaggaactgaagaaccaacggttcgtacttaa

SEQ ID NO: 119 - protein, 569 aa, Saccharomyces cerevisiae
MSFQIETVPTKPYEDQKPGTSGLRKKTKVFKDEPNYTENFIQSIMEAIPEGSKGATLVVGGDGRYYNDVILHKIAAIGAANGIKKLVIGQHGLLSTPAASHIMRTYEEKCTGGIILTASHNPGGPENDMGIKYNLSNGGPAPESVTNAIWEISKKLTSYKIIKDFPELDLGTIGKNKKYGPLLVDIIDITKDYVNFLKEIFDFDLIKKFIDNQRSTKNWKLLFDSMNGVTGPYGKAIFVDEFGLPADEVLQNWHPSPDFGGMHPDPNLTYASSLVKRVDREKIEFGAASDGDGDRNMIYGYGPSFVSPGDSVAIIAEYAAEIPYFAKQGIYGLARSFPTSGAIDRVAKAHGLNCYEVPTGWKFFCALFDAKKLSICGEESFGTGSNHVREKDGVWAIMAWLNILAIYNKHHPENEASIKTIQNEFWAKYGRTFFTRYDFEKVETEKANKIVDQLRAYVTKSGVVNSAFPADESLKVTDCGDFSYTDLDGSVSDHQGLYVKLSNGARFVLRLSGTGSSGATIRLYIEKYCDDKSQYQKTAEEYLKPIINSVIKFLNFKQVLGTEEPTVRT

SEQ ID NO: 120 - DNA, 1500 bp, Saccharomyces cerevisiae
atgtccactaagaagcacaccaaaacacattccacttatgcattcgagagcaacacaaacagcgttgctgcctcacaaatgagaaacgccttaaacaagttggcggactctagtaaacttgacgatgctgctcgcgctaagtttgagaacgaactggattcgtttttcacgcttttcaggagatatttggtagagaagtcttctagaaccaccttggaatgggacaagatcaagtctcccaacccggatgaagtggttaagtatgaaattatttctcagcagcccgagaatgtctcaaacctttccaaattggctgttttgaagttgaacggtgggctgggtacctccatgggctgcgttggccctaaatctgttattgaagtgagagagggaaacacctttttggatttgtctgttcgtcaaattgaatacttgaacagacagtacgatagcgacgtgccattgttattgatgaattctttcaacactgacaaggatacggaacacttgattaagaagtattccgctaacagaatcagaatcagatctttcaatcaatccaggttcccaagagtctacaaggattctttattgcctgtccccaccgaatacgattctccactggatgcttggtatccaccaggtcacggtgatttgtttgaatctttacacgtatctggtgaactggatgccttaattgcccaaggaagagaaatattatttgtttctaacggtgacaacttgggtgctaccgtcgacttaaaaattttaaaccacatgatcgagactggtgccgaatatataatggaattgactgataagaccagagccgatgttaaaggtggtactttgatttcttacgatggtcaagtccgtttattggaagtcgcccaagttccaaaagaacacattgacgaattcaaaaatatcagaaagtttaccaacttcaacacgaataacttatggatcaatctgaaagcagtaaagaggttgatcgaatcgagcaatttggagatggaaatcattccaaaccaaaaaactataacaagagacggtcatgaaattaatgtcttacaattagaaaccgcttgtggtgctgctatcaggcattttgatggtgctcacggtgttgtcgttccaagatcaagattcttgcctgtcaagacctgttccgatttgttgctggttaaatcagatctattccgtctggaacacggttctttgaagttagacccatcccgttttggtccaaacccattaatcaagttgggctcgcatttcaaaaaggtttctggttttaacgcaagaatccctcacatcccaaaaatcgtcgagctagatcatttgaccatcactggtaacgtctttttaggtaaagatgtcactttgaggggtactgtcatcatcgtttgctccgacggtcataaaatcgatattccaaacggctccatattggaaaatgttgtcgttactggtaatttgcaaatcttggaacattga

SEQ ID NO: 121 - protein, 499 aa, Saccharomyces cerevisiae
MSTKKHTKTHSTYAFESNTNSVAASQMRNALNKLADSSKLDDAARAKFENELDSFFTLFRRYLVEKSSRTTLEWDKIKSPNPDEVVKYEIISQQPENVSNLSKLAVLKLNGGLGTSMGCVGPKSVIEVREGNTFLDLSVRQIEYLNRQYDSDVPLLLMNSFNTDKDTEHLIKKYSANRIRIRSFNQSRFPRVYKDSLLPVPTEYDSPLDAWYPPGHGDLFESLHVSGELDALIAQGREILFVSNGDNLGATVDLKILNHMIETGAEYIMELTDKTRADVKGGTLISYDGQVRLLEVAQVPKEHIDEFKNIRKFTNFNTNNLWINLKAVKRLIESSNLEMEIIPNQKTITRDGHEINVLQLETACGAAIRHFDGAHGVVVPRSRFLPVKTCSDLLLVKSDLFRLEHGSLKLDPSRFGPNPLIKLGSHFKKVSGFNARIPHIPKIVELDHLTITGNVFLGKDVTLRGTVIIVCSDGHKIDIPNGSILENVVVTGNLQILEH

SEQ ID NO: 122 - DNA, 462 bp, Saccharomyces cerevisiae
atgtctagtcaaacagaaagaacttttattgcggtaaaaccagatggtgtccagaggggcttagtatctcaaattctatctcgttttgaaaaaaaaggttacaaactagttgctattaaattagttaaagcggatgataaattactagagcaacattacgcagagcatgttggtaaaccatttttcccaaagatggtatcctttatgaagtctggtcccattttggccacggtctgggagggaaaagatgtggttagacaaggaagaactattcttggtgctactaatcctttgggcagtgcaccaggtaccattagaggtgatttcggtattgacctaggcagaaacgtctgtcacggcagtgattctgttgatagcgctgaacgtgaaatcaatttgtggtttaagaaggaagagttagttgattgggaatctaatcaagctaagtggatttatgaatga

SEQ ID NO: 123 - protein, 153 aa, Saccharomyces cerevisiae
MSSQTERTFIAVKPDGVQRGLVSQILSRFEKKGYKLVAIKLVKADDKLLEQHYAEHVGKPFFPKMVSFMKSGPILATVWEGKDVVRQGRTILGATNPLGSAPGTIRGDFGIDLGRNVCHGSDSVDSAEREINLWFKKEELVDWESNQAKWIYE

SEQ ID NO: 124 - DNA, 1413 bp, Stevia rebaudiana
atggctgctgctgatactgaaaagttgaacaatttgagatccgccgtttctggtttgacccaaatttctgataacgaaaagtccggtttcatcaacttggtcagtagatatttgtctggtgaagctcaacacgttgaatggtctaaaattcaaactccaaccgataagatcgttgttccatacgatactttgtctgctgttccagaagatgctgctcaaacaaaatctttgttggataagttggtcgtcttgaagttgaacggtggtttgggtactactatgggttgtactggtccaaagtctgttatcgaagttagaaacggtttgaccttcttggatttgatcgtcatccaaatcgaatccttgaacaagaagtacggttgttctgttcctttgttgttgatgaactctttcaacacccatgaagatacccaaaagatcgtcgaaaagtactccggttctaacattgaagttcacaccttcaatcaatcccaatacccaagattggttgtcgatgaatttttgccattgccatctaaaggtgaaactggtaaagatggttggtatccaccaggtcatggtgatgtttttccatccttgatgaattccggtaagttggatgctttgttgtcccaaggtaaagaatacgttttcgttgccaactctgataacttgggtgcagttgttgatttgaagatcttgaaccacttgatccaaaacaagaacgaatactgcatggaagttactccaaagactttggctgatgttaagggtggtactttgatttcttacgatggtaaggttcaattattggaaatcgcccaagttccagatgaacacgttaatgaattcaagtccatcgaaaagtttaagatctttaacactaacaacttgtgggtcaacttgaacgccattaagagattggttcaagctgatgctttgaagatggaaattattccaaatccaaaagaagtcaacggtgtcaaggtattgcaattggaaactgctgctggtgctgctattaagtttttcgataatgccatcggtatcaacgtcccaagatctagatttttgcctgttaaggcttcctctgacttgttgttagttcaatcagacttgtacaccgaaaaggatggttacgttattagaaacccagctagaaaggatccagctaacccatctattgaattgggtccagaattcaaaaaggtcggtgatttcttgaagagattcaagtctatcccatccatcatcgaattggactcattgaaagtttctggtgatgtctggtttggttccaacgttgttttgaaaggtaaggttgttgttgctgccaaatccggtgaaaaattggaaattccagatggtgccttgattgaaaacaaagaagttcatggtgcctccgacatttga

SEQ ID NO: 125 - protein, 470 aa, Stevia rebaudiana
MAAADTEKLNNLRSAVSGLTQISDNEKSGFINLVSRYLSGEAQHVEWSKIQTPTDKIVVPYDTLSAVPEDAAQTKSLLDKLVVLKLNGGLGTTMGCTGPKSVIEVRNGLTFLDLIVIQIESLNKKYGCSVPLLLMNSFNTHEDTQKIVEKYSGSNIEVHTFNQSQYPRLVVDEFLPLPSKGETGKDGWYPPGHGDVFPSLMNSGKLDALLSQGKEYVFVANSDNLGAVVDLKILNHLIQNKNEYCMEVTPKTLADVKGGTLISYDGKVQLLEIAQVPDEHVNEFKSIEKFKIFNTNNLWVNLNAIKRLVQADALKMEIIPNPKEVNGVKVLQLETAAGAAIKFFDNAIGINVPRSRFLPVKASSDLLLVQSDLYTEKDGYVIRNPARKDPANPSIELGPEFKKVGDFLKRFKSIPSIIELDSLKVSGDVWFGSNVVLKGKVVVAAKSGEKLEIPDGALIENKEVHGASDI

SEQ ID NO: 126 - DNA, 1551 bp, Aureobasidium pullulans
atgtcctctgaaatggctactcatttgaaacctaatggtggtgccgaattcgaaaaaagacatcatggtaagacccaatcccatgttgcttttgaaaacacttctacatctgttgctgcctcccaaatgagaaatgctttgaatactttgtgcgattccgttactgatccagctgaaaagcaaagattcgaaaccgaaatggataacttcttcgccttgtttagaagatacttgaacgataaggctaagggtaacgaaatcgaatggtctagaattgctccaccaaaaccagaacaagttgttgcttatcaagacttgcctgaacaagaatccgttgaattcttgaacaaattggccgtcttgaagttgaatggtggtttgggtacttctatgggttgtgttggtccaaagtctgttatcgaagttagagatggtatgtccttcttggatttgtccgttagacaaatcgaatacttgaatagaacctacggtgttaacgttccattcgtcttgatgaattctttcaacactgatgctgataccgccaacattatcaaaaagtacgaaggtcacaacatcgacatcatgaccttcaatcaatctagatacccaagaatcttgaaggattctttgttgccagctccaaaatctgccaactctcaaatttctgattggtatccaccaggtcatggtgacgtttttgaatccttgtacaactctggtatcttggataagttgttggaaagaggtgtcgaaatcgttttcttgtccaatgctgataatttgggtgccgttgttgatttgaagatcttgcaacatatggttgataccaaggccgaatatatcatggaattgactgataagactaaggccgatgttaagggtggtactattattgactatgaaggtcaagccagattattggaaattgcccaagttccaaaagaacacgtcaacgaattcaagtccatcaagaagtttaagtacttcaacaccaacaacatctggatgaacttgagagctgttaagagaatcgtcgaaaacaacgaattggccatggaaattatcccaaacggtaaatctattccagccgacaaaaaaggtgaagccgatgtttctatagttcaattggaaactgctgttggtgctgccattagacattttaacaatgctcatggtgtcaacgtcccaagaagaagatttttgccagttaagacctgctccgatttgatgttggttaagtctgacttgtacactttgaagcacggtcaattgattatggacccaaatagatttggtccagccccattgattaagttgggtggtgattttaagaaggtttcctcattccaatccagaatcccatccattcctaaaatcttggaattggatcatttgaccattaccggtccagttaacttgggtagaggtgttacttttaagggtactgttattatcgttgcctccgaaggtcaaaccattgatattccacctggttccattttggaaaacgttgttgttcaaggttccttgagattattagaacattaa

SEQ ID NO: 127 - protein, 516 aa, Aureobasidium pullulans
MSSEMATHLKPNGGAEFEKRHHGKTQSHVAFENTSTSVAASQMRNALNTLCDSVTDPAEKQRFETEMDNFFALFRRYLNDKAKGNEIEWSRIAPPKPEQVVAYQDLPEQESVEFLNKLAVLKLNGGLGTSMGCVGPKSVIEVRDGMSFLDLSVRQIEYLNRTYGVNVPFVLMNSFNTDADTANIIKKYEGHNIDIMTFNQSRYPRILKDSLLPAPKSANSQISDWYPPGHGDVFESLYNSGILDKLLERGVEIVFLSNADNLGAVVDLKILQHMVDTKAEYIMELTDKTKADVKGGTIIDYEGQARLLEIAQVPKEHVNEFKSIKKFKYFNTNNIWMNLRAVKRIVENNELAMEIIPNGKSIPADKKGEADVSIVQLETAVGAAIRHFNNAHGVNVPRRRFLPVKTCSDLMLVKSDLYTLKHGQLIMDPNRFGPAPLIKLGGDFKKVSSFQSRIPSIPKILELDHLTITGPVNLGRGVTFKGTVIIVASEGQTIDIPPGSILENVVVQGSLRLLEH

SEQ ID NO: 128 - DNA, 1410 bp, Arabidopsis thaliana
atggctgctactactgaaaacttgccacaattgaaatctgccgttgatggtttgactgaaatgtccgaatctgaaaagtccggtttcatctctttggtcagtagatatttgtctggtgaagcccaacatatcgaatggtctaaaattcaaactccaaccgacgaaatcgttgtcccatacgaaaaaatgactccagtttctcaagatgtcgccgaaactaagaatttgttggataagttggtcgtcttgaagttgaatggtggtttgggtactactatgggttgtactggtccaaagtctgttatcgaagttagagatggtttaaccttcttggacttgatcgtcatccaaatcgaaaacttgaacaacaagtacggttgcaaggttccattggtcttgatgaattctttcaacacccatgatgatacccacaagatcgttgaaaagtacaccaactccaacgttgatatccacaccttcaatcaatctaagtacccaagagttgttgccgatgaatttgttccatggccatctaaaggtaagactgacaaagaaggttggtatccaccaggtcatggtgatgtttttccagctttaatgaactccggtaagttggatactttcttgtcccaaggtaaagaatacgttttcgttgccaactctgataacttgggtgctatagttgatttgaccatcttgaagcacttgatccaaaacaagaacgaatactgcatggaagttactccaaagactttggctgatgttaagggtggtactttgatttcttacgaaggtaaggttcaattattggaaatcgcccaagttccagatgaacacgttaatgaattcaagtccatcgaaaagttcaagatcttcaacaccaacaacttgtgggttaacttgaaggccatcaagaaattggttgaagctgatgctttgaagatggaaattatcccaaacccaaaagaagttgacggtgttaaggtattgcaattggaaactgctgctggtgctgctattagatttttcgataatgccatcggtgttaacgtcccaagatctagatttttgccagttaaggcttcctccgatttgttgttggttcaatctgacttgtacaccttggttgacggttttgttacaagaaacaaggctagaactaacccatccaacccatctattgaattgggtccagaattcaaaaaggttgccacattcttgtccagattcaagtctattccatccatcgtcgaattggactcattgaaagtttctggtgatgtctggtttggttcctctatagttttgaagggtaaggttactgttgctgctaaatctggtgttaagttggaaattccagatagagccgttgtcgaaaacaaaaacattaacggtcctgaagatttgtga

SEQ ID NO: 129 - protein, 469 aa, Arabidopsis thaliana
MAATTENLPQLKSAVDGLTEMSESEKSGFISLVSRYLSGEAQHIEWSKIQTPTDEIVVPYEKMTPVSQDVAETKNLLDKLVVLKLNGGLGTTMGCTGPKSVIEVRDGLTFLDLIVIQIENLNNKYGCKVPLVLMNSFNTHDDTHKIVEKYTNSNVDIHTFNQSKYPRVVADEFVPWPSKGKTDKEGWYPPGHGDVFPALMNSGKLDTFLSQGKEYVFVANSDNLGAIVDLTILKHLIQNKNEYCMEVTPKTLADVKGGTLISYEGKVQLLEIAQVPDEHVNEFKSIEKFKIFNTNNLWVNLKAIKKLVEADALKMEIIPNPKEVDGVKVLQLETAAGAAIRFFDNAIGVNVPRSRFLPVKASSDLLLVQSDLYTLVDGFVTRNKARTNPSNPSIELGPEFKKVATFLSRFKSIPSIVELDSLKVSGDVWFGSSIVLKGKVTVAAKSGVKLEIPDRAVVENKNINGPEDL

SEQ ID NO: 130 - DNA, 909 bp, Escherichia coli
atggctgctattaacaccaaggttaagaaggctgttattccagttgctggtttgggtactagaatgttgccagctacaaaagccattccaaaagaaatgttaccattggtcgataagccattgatccaatacgttgtcaacgaatgtattgctgctggtattaccgaaatcgttttggttactcactcctccaagaactccattgaaaatcatttcgacacctcattcgaattggaagccatgttggaaaagagagtcaagagacaattattggacgaagtccaatctatttgcccaccacatgttactatcatgcaagttagacaaggtttggctaaaggtttgggtcatgctgttttgtgtgctcatccagttgttggtgatgaaccagttgcagttattttgccagatgttatcttggacgaatacgaatccgatttgtctcaagataacttggctgaaatgatcagaagattcgacgaaactggtcactcccaaattatggttgaacctgttgctgatgttactgcttatggtgttgttgattgcaagggtgttgaattggctccaggtgaatctgttccaatggttggtgttgtagaaaagccaaaagctgatgttgctccatctaatttggctatcgttggtagatatgttttgtccgctgatatttggcctttgttggctaaaactccaccaggtgctggtgacgaaattcaattgactgatgctatcgacatgttgatcgaaaaagaaaccgttgaagcctaccacatgaagggtaaatctcatgattgtggtaacaagttgggttacatgcaagcttttgttgaatacggtatcagacataacaccttaggtactgaattcaaggcttggttggaagaagaaatgggtatcaagaagtaa

SEQ ID NO: 131 - protein, 302 aa, Escherichia coli
MAAINTKVKKAVIPVAGLGTRMLPATKAIPKEMLPLVDKPLIQYVVNECIAAGITEIVLVTHSSKNSIENHFDTSFELEAMLEKRVKRQLLDEVQSICPPHVTIMQVRQGLAKGLGHAVLCAHPVVGDEPVAVILPDVILDEYESDLSQDNLAEMIRRFDETGHSQIMVEPVADVTAYGVVDCKGVELAPGESVPMVGVVEKPKADVAPSNLAIVGRYVLSADIWPLLAKTPPGAGDEIQLTDAIDMLIEKETVEAYHMKGKSHDCGNKLGYMQAFVEYGIRHNTLGTEFKAWLEEEMGIKK

SEQ ID NO: 132 - DNA, 1416 bp, Rubus suavissimus
atggctgctgttgctactgataagatctctaagttgaagtctgaagttgctgccttgtcccaaatttctgaaaacgaaaagtccggtttcatcaacttggtcagtagatatttgtctggtactgaagctactcacgttgaatggtctaaaattcaaactccaaccgatgaagttgttgttccatatgatactttggctccaactccagaagatccagctgaaactaagaagttgttagataagttggtcgtcttgaagttgaacggtggtttgggtactactatgggttgtactggtccaaagtctgttatcgaagttagaaacggtttgaccttcttggatttgatcgtcattcaaatcgaaaccttgaacaacaagtacggttgtaacgttcctttgttgttgatgaactctttcaacacccatgatgacaccttcaagatcgttgaaagatacaccaagtccaacgttcaaatccataccttcaatcaatcccaatacccaagattggttgtcgaagataattctccattgccatctaagggtcaaactggtaaagatggttggtatccaccaggtcatggtgatgtttttccatctttgagaaactccggtaagttggatttgttgttatcccaaggtaaagaatacgttttcatctccaactctgataacttgggtgcagttgttgatttgaagatcttgtcccatttggtccaaaaaaagaacgaatactgcatggaagttaccccaaaaactttggctgatgttaagggtggtactttgatttcttacgaaggtagaacccaattattggaaattgcccaagttccagatcaacacgttaacgaattcaagtccatcgaaaagttcaagatctttaacaccaacaatttgtgggtcaacttgaacgccattaagagattagttgaagctgatgccttgaaaatggaaatcatcccaaatccaaaagaagtcgacggtattaaggtcttgcaattggaaactgctgctggtgctgctattagatttttcaatcatgccatcggtatcaacgtcccaagatctagatttttgccagttaaggctacctccgatttgttattggttcaatctgacttgtacaccgtcgaagatggtttcgttattagaaacactgctagaaagaatccagccaacccatctgttgaattgggtccagaattcaaaaaggttgccaacttcttgtccagattcaagtctattccatccatcatcgaattggactcattgaaggttgttggtgatgtatggtttggtgctggtgttgttttgaaaggtaaggttactattactgctaagccaggtgttaagttggaaattccagataaggctgtcttggaaaacaaggatattaacggtcctgaagatttgtga

SEQ ID NO: 133 - protein, 471 aa, Rubus suavissimus
MAAVATDKISKLKSEVAALSQISENEKSGFINLVSRYLSGTEATHVEWSKIQTPTDEVVVPYDTLAPTPEDPAETKKLLDKLVVLKLNGGLGTTMGCTGPKSVIEVRNGLTFLDLIVIQIETLNNKYGCNVPLLLMNSFNTHDDTFKIVERYTKSNVQIHTFNQSQYPRLVVEDNSPLPSKGQTGKDGWYPPGHGDVFPSLRNSGKLDLLLSQGKEYVFISNSDNLGAVVDLKILSHLVQKKNEYCMEVTPKTLADVKGGTLISYEGRTQLLEIAQVPDQHVNEFKSIEKFKIFNTNNLWVNLNAIKRLVEADALKMEIIPNPKEVDGIKVLQLETAAGAAIRFFNHAIGINVPRSRFLPVKATSDLLLVQSDLYTVEDGFVIRNTARKNPANPSVELGPEFKKVANFLSRFKSIPSIIELDSLKVVGDVWFGAGVVLKGKVTITAKPGVKLEIPDKAVLENKDINGPEDL

SEQ ID NO: 134 - DNA, 1422 bp, Hordeum vulgare
atggctgctgctgcagttgctgctgattctaaaattgatggtttgagagatgctgttgccaagttgggtgaaatttctgaaaacgaaaaggccggtttcatctccttggtttctagatatttgtctggtgaagccgaacaaatcgaatggtctaaaattcaaactccaaccgatgaagttgttgttccatatgatactttggctccaccacctgaagatttggatgctatgaaggctttgttggataagttggttgtcttgaagttgaatggtggtttgggtactactatgggttgtactggtccaaagtctgttatcgaagttagaaacggtttcaccttcttggatttgatcgttatccaaattgaatccttgaacaagaagtacggttgctctgttcctttgttgttgatgaactctttcaacacccatgatgacacccaaaagatcgttgaaaagtactccaactccaacatcgaaatccacaccttcaatcaatctcaatacccaagaatcgtcaccgaagattttttgccattgccatctaaaggtcaaactggtaaagatggttggtatccaccaggtcatggtgatgtttttccatctttgaacaactccggtaagttggataccttgttgtctcaaggtaaagaatacgttttcgttgccaactctgataacttgggtgctatcgttgatattaagatcttgaaccacttgatccacaatcaaaacgaatactgcatggaagttactccaaagactttggctgatgttaagggtggtactttgatttcttacgaaggtagagttcaattattggaaatcgcccaagttccagatgaacacgttgatgaattcaagtccatcgaaaagttcaaaatcttcaacaccaacaacttgtgggttaacttgaaggccattaagagattggttgatgctgaagctttgaaaatggaaatcatcccaaaccctaaagaagttgacggtgttaaggtattgcaattggaaactgctgctggtgctgctattagattctttgaaaaagccatcggtatcaacgtcccaagatctagatttttgccagttaaggctacctctgacttgttgttggttcaatcagacttgtacaccttggttgacggttacgttattagaaatccagctagagttaagccatccaacccatctattgaattgggtccagaattcaagaaggtcgctaatttcttggctagattcaagtctatcccatccatcgttgaattggactcattgaaagtttctggtgatgtctcttttggttccggtgttgttttgaagggtaatgttactattgctgctaaggctggtgttaagttggaaattccagatggtgctgttttggaaaacaaggatattaacggtccagaagatatttga

SEQ ID NO: 135 - protein, 473 aa, Hordeum vulgare
MAAAAVAADSKIDGLRDAVAKLGEISENEKAGFISLVSRYLSGEAEQIEWSKIQTPTDEVVVPYDTLAPPPEDLDAMKALLDKLVVLKLNGGLGTTMGCTGPKSVIEVRNGFTFLDLIVIQIESLNKKYGCSVPLLLMNSFNTHDDTQKIVEKYSNSNIEIHTFNQSQYPRIVTEDFLPLPSKGQTGKDGWYPPGHGDVFPSLNNSGKLDTLLSQGKEYVFVANSDNLGAIVDIKILNHLIHNQNEYCMEVTPKTLADVKGGTLISYEGRVQLLEIAQVPDEHVDEFKSIEKFKIFNTNNLWVNLKAIKRLVDAEALKMEIIPNPKEVDGVKVLQLETAAGAAIRFFEKAIGINVPRSRFLPVKATSDLLLVQSDLYTLVDGYVIRNPARVKPSNPSIELGPEFKKVANFLARFKSIPSIVELDSLKVSGDVSFGSGVVLKGNVTIAAKAGVKLEIPDGAVLENKDINGPEDI

SEQ ID NO: 136 - DNA, 1404 bp, Oryza sativa
atggctgacgaaaaattggccaaattgagagaagctgttgctggtttgtctcaaatctctgataacgaaaagtccggtttcatttccttggttgctagatatttgtccggtgaagaagaacatgttgaatgggctaaaattcataccccaaccgatgaagttgttgttccatatgatactttggaagctccaccagaagatttggaagaaacaaaaaagttgttgaacaagttggccgtcttgaagttgaatggtggtttgggtactactatgggttgtactggtccaaagtctgttatcgaagttagaaacggtttcaccttcttggatttgatcgtcatccaaatcgaatccttgaacaaaaagtacggttccaacgttcctttgttgttgatgaactctttcaacacccatgaagataccttgaagatcgttgaaaagtacaccaactccaacatcgaagttcacaccttcaatcaatctcaatacccaagagttgttgccgatgaatttttgccatggccatctaaaggtaagacttgtaaagatggttggtatccaccaggtcatggtgatatttttccatccttgatgaacagtggtaagttggacttgttgttgtcccaaggtaaagaatacgttttcattgccaactccgataacttgggtgctatagttgatatgaagattttgaaccacttgatccacaagcaaaacgaatactgtatggaagttactccaaagactttggctgatgttaagggtggtactttgatctcttacgaagataaggttcaattattggaaatcgcccaagttccagatgctcatgttaatgaattcaagtccatcgaaaagttcaagatctttaacaccaacaacttgtgggttaacttgaaggccattaagagattagttgaagctgacgctttgaagatggaaattatcccaaacccaaaagaagttgacggtgttaaggtattgcaattggaaactgctgctggtgctgctattagatttttcgatcatgctatcggtatcaacgtcccaagatctagatttttaccagttaaggctacctccgacttgcaattagttcaatctgacttgtacaccttggttgatggtttcgttactagaaatccagctagaactaatccatccaacccatctattgaattgggtccagaattcaagaaggttggttgttttttgggtagattcaagtctatcccatccatcgttgaattggacactttgaaagtttctggtgatgtttggttcggttcctccattacattgaaaggtaaggttactattaccgctcaaccaggtgttaagttggaaattccagatggtgctgtcatcgaaaacaaggatattaacggtcctgaagatttgtga

SEQ ID NO: 137 - protein, 467 aa, Oryza sativa
MADEKLAKLREAVAGLSQISDNEKSGFISLVARYLSGEEEHVEWAKIHTPTDEVVVPYDTLEAPPEDLEETKKLLNKLAVLKLNGGLGTTMGCTGPKSVIEVRNGFTFLDLIVIQIESLNKKYGSNVPLLLMNSFNTHEDTLKIVEKYTNSNIEVHTFNQSQYPRVVADEFLPWPSKGKTCKDGWYPPGHGDIFPSLMNSGKLDLLLSQGKEYVFIANSDNLGAIVDMKILNHLIHKQNEYCMEVTPKTLADVKGGTLISYEDKVQLLEIAQVPDAHVNEFKSIEKFKIFNTNNLWVNLKAIKRLVEADALKMEIIPNPKEVDGVKVLQLETAAGAAIRFFDHAIGINVPRSRFLPVKATSDLQLVQSDLYTLVDGFVTRNPARTNPSNPSIELGPEFKKVGCFLGRFKSIPSIVELDTLKVSGDVWFGSSITLKGKVTITAQPGVKLEIPDGAVIENKDINGPEDL

SEQ ID NO: 138 - DNA, 1434 bp, Solanum tuberosum
atggctactgctactactttgtctccagctgatgctgaaaagttgaacaatttgaaatctgctgtcgccggtttgaatcaaatctctgaaaacgaaaagtccggtttcatcaacttggttggtagatatttgtctggtgaagcccaacatattgactggtctaaaattcaaactccaaccgatgaagttgttgtcccatatgataagttggctccattgtctgaagatccagctgaaacaaaaaagttgttggacaagttggtcgtcttgaagttgaatggtggtttgggtactactatgggttgtactggtccaaagtctgttatcgaagttagaaacggtttgaccttcttggatttgatcgtcaagcaaattgaagctttgaacgctaagttcggttgttctgttcctttgttgttgatgaactctttcaacacccatgatgacaccttgaagatcgttgaaaagtacgccaactccaacattgatatccacaccttcaatcaatcccaatacccaagattggttaccgaagattttgctccattgccatgtaaaggtaactctggtaaagatggttggtatccaccaggtcatggtgatgtttttccatccttgatgaattccggtaagttggatgctttgttggctaagggtaaagaatacgttttcgttgccaactctgataacttgggtgctatcgttgatttgaaaatcttgaaccacttgatcttgaacaagaacgaatactgcatggaagttactccaaagactttggctgatgttaagggtggtactttgatttcttacgaaggtaaggttcaattattggaaatcgcccaagttccagatgaacacgttaatgaattcaagtccatcgaaaagtttaagatcttcaacactaacaacttgtgggtcaacttgtctgccattaagagattggttgaagctgatgccttgaaaatggaaattattccaaacccaaaagaagtcgatggtgtcaaagtattgcaattggaaactgctgctggtgctgctattaagtttttcgatagagctattggtgccaacgttccaagatctagatttttgccagttaaggctacctctgacttgttgttggttcaatcagacttgtacactttgactgatgaaggttacgttattagaaacccagctagatccaatccatccaacccatctattgaattgggtccagaattcaagaaggtagccaattttttgggtagattcaagtctatcccatccatcatcgatttggattctttgaaagttactggtgatgtctggtttggttctggtgttactttgaaaggtaaagttaccgttgctgctaagtcaggtgttaagttggaaattccagatggtgctgttattgccaacaaggatattaacggtccagaagatatctaa

SEQ ID NO: 139 - protein, 477 aa, Solanum tuberosum
MATATTLSPADAEKLNNLKSAVAGLNQISENEKSGFINLVGRYLSGEAQHIDWSKIQTPTDEVVVPYDKLAPLSEDPAETKKLLDKLVVLKLNGGLGTTMGCTGPKSVIEVRNGLTFLDLIVKQIEALNAKFGCSVPLLLMNSFNTHDDTLKIVEKYANSNIDIHTFNQSQYPRLVTEDFAPLPCKGNSGKDGWYPPGHGDVFPSLMNSGKLDALLAKGKEYVFVANSDNLGAIVDLKILNHLILNKNEYCMEVTPKTLADVKGGTLISYEGKVQLLEIAQVPDEHVNEFKSIEKFKIFNTNNLWVNLSAIKRLVEADALKMEIIPNPKEVDGVKVLQLETAAGAAIKFFDRAIGANVPRSRFLPVKATSDLLLVQSDLYTLTDEGYVIRNPARSNPSNPSIELGPEFKKVANFLGRFKSIPSIIDLDSLKVTGDVWFGSGVTLKGKVTVAAKSGVKLEIPDGAVIANKDINGPEDI

SEQ ID NO: 140 - DNA, 1818 bp, Arabidopsis thaliana
atgttcttgttggttacctcttgcttcttgccagattctggttcttctgttaaggtcagtttgttcatcttcggtgtctcattggtttctacctctccaattgatggtcaaaaaccaggtacttctggtttgagaaagaaggtcaaggttttcaagcaacctaactacttggaaaacttcgttcaagctactttcaacgctttgactaccgaaaaagttaagggtgctactttggttgtttctggtgatggtagatattactccgaacaagccattcaaatcatcgttaagatggctgctgctaacggtgttagaagagtttgggttggtcaaaactctttgttgtctactccagctgtttccgccattattagagaaagagttggtgctgatggttctaaagctactggtgctttcattttgactgcttctcataatccaggtggtccaactgaagatttcggtattaagtacaacatggaaaatggtggtccagccccagaatctattactgataagatatacgaaaacaccaagaccatcaaagaatacccaattgcagaagatttgccaagagttgatatctctactatcggtatcacttctttcgaaggtcctgaaggtaaattcgacgttgaagtttttgattccgctgatgattacgtcaagttgatgaagtccatcttcgacttcgaatccatcaagaagttgttgtcttacccaaagttcaccttttgttacgatgcattgcatggtgttgctggtgcttatgctcatagaattttcgttgaagaattgggtgctccagaatcctctttattgaactgtgttccaaaagaagattttggtggtggtcatccagatccaaatttgacttatgccaaagaattggttgccagaatgggtttgtctaagactgatgatgctggtggtgaaccacctgaatttggtgctgctgcagatggtgatgctgatagaaatatgatcttgggtaaaagattcttcgtcaccccatctgattccgttgctattattgctgctaatgctgttggtgctattccatacttttcatccggtttgaaaggtgttgctagatctatgccaacttctgctgctttggatgttgttgctaagaatttgggtttgaagttcttcgaagttccaactggttggaaattcttcggtaatttgatggatgcaggtatgtgttctgtttgcggtgaagaatcatttggtactggttccgatcatatcagagaaaaggatggtatttgggctgttttggcttggttgtctattttggctcacaagaacaaagaaaccttggatggtaatgccaagttggttactgttgaagatatcgttagacaacattgggctacttacggtagacattactacactagatacgactacgaaaacgttgatgctacagctgctaaagaattgatgggtttattggtcaagttgcaatcctcattgccagaagttaacaagatcatcaagggtatccatcctgaagttgctaatgttgcttctgctgatgaattcgaatacaaggatccagttgatggttccgtttctaaacatcaaggtatcagatacttgtttgaagatggttccagattggttttcagattgtctggtacaggttctgaaggtgctactattagattgtacatcgaacaatacgaaaaggacgcctctaagattggtagagattctcaagatgctttgggtccattggttgatgttgctttgaagttgtccaagatgcaagaattcactggtagatcttctccaaccgttattacctga

SEQ ID NO: 141 - protein, 605 aa, Arabidopsis thaliana
MFLLVTSCFLPDSGSSVKVSLFIFGVSLVSTSPIDGQKPGTSGLRKKVKVFKQPNYLENFVQATFNALTTEKVKGATLVVSGDGRYYSEQAIQIIVKMAAANGVRRVWVGQNSLLSTPAVSAIIRERVGADGSKATGAFILTASHNPGGPTEDFGIKYNMENGGPAPESITDKIYENTKTIKEYPIAEDLPRVDISTIGITSFEGPEGKFDVEVFDSADDYVKLMKSIFDFESIKKLLSYPKFTFCYDALHGVAGAYAHRIFVEELGAPESSLLNCVPKEDFGGGHPDPNLTYAKELVARMGLSKTDDAGGEPPEFGAAADGDADRNMILGKRFFVTPSDSVAIIAANAVGAIPYFSSGLKGVARSMPTSAALDVVAKNLGLKFFEVPTGWKFFGNLMDAGMCSVCGEESFGTGSDHIREKDGIWAVLAWLSILAHKNKETLDGNAKLVTVEDIVRQHWATYGRHYYTRYDYENVDATAAKELMGLLVKLQSSLPEVNKIIKGIHPEVANVASADEFEYKDPVDGSVSKHQGIRYLFEDGSRLVFRLSGTGSEGATIRLYIEQYEKDASKIGRDSQDALGPLVDVALKLSKMQEFTGRSSPTVIT

SEQ ID NO: 142 - DNA, 1641 bp, Escherichia coli
atggccattcataatagagctggtcaaccagcacaacaatccgatttgattaacgttgctcaattgaccgcccaatattacgttttgaaacctgaagctggtaacgctgaacatgctgttaagtttggtacttctggtcatagaggttctgctgctagacattcttttaacgaaccacatattttggctatcgctcaagctattgctgaagaaagagctaagaacggtattactggtccatgttacgttggtaaagatacccatgctttgtctgaaccagctttcatttctgttttggaagttttggctgctaacggtgttgatgttatcgttcaagaaaacaacggtttcactccaactccagctgtttctaatgctattttggttcacaacaaaaagggtggtccattggctgatggtatagttattactccatctcataacccacctgaagatggtggtattaagtacaatccaccaaatggtggtccagctgatacaaatgttactaaggttgttgaagatagagccaacgctttgttagctgatggtttgaaaggtgtcaagagaatctctttggatgaagctatggcttcaggtcatgtcaaagaacaagatttggttcaaccattcgttgaaggtttggctgatatagttgatatggctgctattcaaaaggctggtttgactttgggtgttgatccattgggtggttctggtattgaatactggaaaagaatcggtgaatattacaacttgaacttgaccatcgtcaacgatcaagttgaccaaactttcagattcatgcacttggataaggatggtgctattagaatggactgttcttctgaatgtgctatggctggtttattggctttgagagataagttcgatttggcttttgctaacgatccagattacgatagacatggtatcgttactccagcaggtttgatgaatccaaatcattacttggctgttgccatcaactacttgtttcaacatagaccacaatggggtaaggatgttgctgttggtaaaactttggtttcctccgctatgatcgatagagttgttaacgatttgggtagaaagttggttgaagttccagttggtttcaagtggtttgttgacggtttgtttgatggttcttttggttttggtggtgaagaatctgctggtgcttcatttttgagatttgatggtactccatggtccactgacaaagatggtattatcatgtgtttgttggctgctgaaattactgctgttactggtaagaatccacaagaacactacaacgaattggctaagagatttggtgctccatcttacaatagattgcaagctgctgctacttctgctcaaaaagctgctttatctaagttgtccccagaaatggtttctgcttctactttagctggtgatccaattacagctagattgactgctgctccaggtaatggtgcttctattggtggtttaaaggttatgactgataacggttggtttgctgcaagaccatctggtactgaagatgcttacaaaatctactgcgaatccttcttgggtgaagaacatagaaagcaaattgaaaaagaagccgtcgaaatcgtcagtgaagttttgaagaatgcctaa

SEQ ID NO: 143 - protein, 546 aa, Escherichia coli
MAIHNRAGQPAQQSDLINVAQLTAQYYVLKPEAGNAEHAVKFGTSGHRGSAARHSFNEPHILAIAQAIAEERAKNGITGPCYVGKDTHALSEPAFISVLEVLAANGVDVIVQENNGFTPTPAVSNAILVHNKKGGPLADGIVITPSHNPPEDGGIKYNPPNGGPADTNVTKVVEDRANALLADGLKGVKRISLDEAMASGHVKEQDLVQPFVEGLADIVDMAAIQKAGLTLGVDPLGGSGIEYWKRIGEYYNLNLTIVNDQVDQTFRFMHLDKDGAIRMDCSSECAMAGLLALRDKFDLAFANDPDYDRHGIVTPAGLMNPNHYLAVAINYLFQHRPQWGKDVAVGKTLVSSAMIDRVVNDLGRKLVEVPVGFKWFVDGLFDGSFGFGGEESAGASFLRFDGTPWSTDKDGIIMCLLAAEITAVTGKNPQEHYNELAKRFGAPSYNRLQAAATSAQKAALSKLSPEMVSASTLAGDPITARLTAAPGNGASIGGLKVMTDNGWFAARPSGTEDAYKIYCESFLGEEHRKQIEKEAVEIVSEVLKNA

SEQ ID NO: 144 - DNA, 1749 bp, Rubus suavissimus
atgtcctccggtaagattaagagagttcaaactactccattcgacggtcaaaaaccaggtacttctggtttgagaaagaaggttaaggttttcacccaacctaactacttgcaaaacttcgttcaatctaccttcaacgctttgccatctgataaggtaaaaggtgctagattggttgtttctggtgatggtagatacttctccaaagaagccattcaaatcatcattaagatggctgctggtaacggtgttaagtctgtttgggttggtcaaaatggtttgttgtctactccagctgtttctgctgttgttagagaaagagttggtgctgatggttgtaaagcttctggtgctttcattttgactgcttctcataatccaggtggtccaaatgaagatttcggtatcaagtacaacatggaaaatggtggtccagctccagaatctattaccaacaaaatctacgaaaacaccacccaaatcaaagaatacttgaccgttgatttgccagaagttgatattactaagccaggtgttactaccttcgaagttgaaggtggtactttcactgttgatgttttcgattctgcttccgattacgtcaagttgatgaagtccattttcgacttcgaatccatcagaaagttgttgtcctctccaaagttcaccttttgttttgatgcattgcatggtgttggtggtgcttacgctaaaagaattttcgttgaagaattgggtgccaaagaatcctctttgttgaactgtgttcctaaagaagattttggtggtggtcatccagatccaaatttgacatatgctaaagaattggtcgccagaatgggtttgtctaagtctaatactcaaaacgaaccaccagaatttggtgctgctgcagatggtgatgctgatagaaatatggttttgggtaagagattcttcgttaccccatctgattccgttgctattattgctgctaatgctgttgaagctatcccatacttttctactggtttgaaaggtgttgctagatctatgccaacttctgctgctttggatgttgttgctaaacacttgaacttgaagttcttcgaagtaccaactggttggaagtttttcggtaatttgatggatgctggtttgtgttctgtttgcggtgaagaatcttttggtactggttccgatcatatcagagaaaaggatggtatttgggctgttttggcttggttgtcaattattgccatcaagaacaaggataacatcggtggtgataagttggttaccgttgaagatatcgttagaaaacattgggctacttacggtagacattactacactagatacgattacgaaaacgttgatgctggtaaggctaaagatttgatggcatcattggtcaacttgcaatcatctttgcctgaagttaacaagatcgttaagggtatctgttccgatgttgcaaatgttgttggtgccgatgaattcgaatacaaggattctgttgatggttccatctccaaacatcaaggtatcagatacttgttcgaagatggttcaagattggttttcagattgtctggtacaggttctgaaggtgctactattagattgtacatcgaacaatacgaaaatgacccatccaagatctccagagaatcttctgaagctttggctccattggttgaagttgctttgaaattgtccaagatgcaagaattcactggtagatcagctccaactgttattacctga

SEQ ID NO: 145 - protein, 582 aa, Rubus suavissimus
MSSGKIKRVQTTPFDGQKPGTSGLRKKVKVFTQPNYLQNFVQSTFNALPSDKVKGARLVVSGDGRYFSKEAIQIIIKMAAGNGVKSVWVGQNGLLSTPAVSAVVRERVGADGCKASGAFILTASHNPGGPNEDFGIKYNMENGGPAPESITNKIYENTTQIKEYLTVDLPEVDITKPGVTTFEVEGGTFTVDVFDSASDYVKLMKSIFDFESIRKLLSSPKFTFCFDALHGVGGAYAKRIFVEELGAKESSLLNCVPKEDFGGGHPDPNLTYAKELVARMGLSKSNTQNEPPEFGAAADGDADRNMVLGKRFFVTPSDSVAIIAANAVEAIPYFSTGLKGVARSMPTSAALDVVAKHLNLKFFEVPTGWKFFGNLMDAGLCSVCGEESFGTGSDHIREKDGIWAVLAWLSIIAIKNKDNIGGDKLVTVEDIVRKHWATYGRHYYTRYDYENVDAGKAKDLMASLVNLQSSLPEVNKIVKGICSDVANVVGADEFEYKDSVDGSISKHQGIRYLFEDGSRLVFRLSGTGSEGATIRLYIEQYENDPSKISRESSEALAPLVEVALKLSKMQEFTGRSAPTVIT

SEQ ID NO: 146 - DNA, 1749 bp, Stevia rebaudiana
atggcctctttcaaggttaacagagttgaatcctctccaatcgaaggtcaaaaaccaggtacttctggtttgagaaagaaggttaaggttttcacccaaccacattacttgcacaacttcgttcaatctactttcaacgctttgtctgccgaaaaagttaagggttctactttggttgtttccggtgatggtagatattactccaaggatgccattcaaatcatcattaagatggctgctgctaacggtgttagaagagtttgggttggtcaaaatggtttgttgtctactccagctgtttctgctgttgttagagaaagagttggtgctgatggttctaaatctaacggtgctttcattttgactgcctctcataatccaggtggtccaaatgaagatttcggtatcaagtacaacatggaaaatggtggtccagctccagaaggtattactgataagatttttgaaaacaccaagaccatcaaagaatacttcattgctgaaggtttgccagacgttgatatttccgctattggtatctcttcattctctggtccagatggtcaattcgatgttgatgttttcgattcctcttccgactacgtcaaattgatgaagtccatcttcgacttccaatccatcaagaagttgattacctccccacaattttctttctgttacgatgctttacatggtgttggtggtgcttatgctaagccaatttttgttgatgaattgggtgccaaagaatcctctttgttgaactgtgttcctaaagaagattttggtggtggtcatccagatccaaatttgacttacgctaaagaattggtttccagaatgggtttgggtaagaatccagattctaatccaccagaatttggtgctgctgcagatggtgatgctgatagaaatatgatcttgggtaaaagattcttcgtcaccccatctgattccgttgctattattgctgctaatgccgttcaatcaatcccatacttttcatccggtttgaaaggtgttgctagatctatgccaacttctgctgctttggatgttgttgctaagtctttgaacttgaagttcttcgaagttccaactggttggaagtttttcggtaatttgatggatgctggtttgtgttctgtttgcggtgaagaatcatttggtactggttccgatcatatcagagaaaaggatggtatttgggctgttttggcttggttgtctattttggctcataagaacaaggacaacttgaacggtggtaacttggttactgttgaagatatcgttaagcaacattgggctacttacggtagacattactacactagatacgactacgaaaacgttgatgctggtgctgcaaaagaattgatggctcatttggttaagttgcaatcctccatctctgatgttaacaccttcattaagggtatcagatccgatgttgctaatgttgcatctgctgatgaattcgaatacaaggatccagttgacggttctatttccaaacatcaaggtattagatacttgtttgaagatggttccagattggttttcagattgtctggtacaggttctgaaggtgctactattagattgtacatcgaacaatacgaaaaggattcctctaagaccggtagagattctcaagaagctttggctccattagttgaagttgccttgaaattgtccaagatgcaagaattcactggtagatctgctccaactgttattacctga

SEQ ID NO: 147 - protein, 582 aa, Stevia rebaudiana
MASFKVNRVESSPIEGQKPGTSGLRKKVKVFTQPHYLHNFVQSTFNALSAEKVKGSTLVVSGDGRYYSKDAIQIIIKMAAANGVRRVWVGQNGLLSTPAVSAVVRERVGADGSKSNGAFILTASHNPGGPNEDFGIKYNMENGGPAPEGITDKIFENTKTIKEYFIAEGLPDVDISAIGISSFSGPDGQFDVDVFDSSSDYVKLMKSIFDFQSIKKLITSPQFSFCYDALHGVGGAYAKPIFVDELGAKESSLLNCVPKEDFGGGHPDPNLTYAKELVSRMGLGKNPDSNPPEFGAAADGDADRNMILGKRFFVTPSDSVAIIAANAVQSIPYFSSGLKGVARSMPTSAALDVVAKSLNLKFFEVPTGWKFFGNLMDAGLCSVCGEESFGTGSDHIREKDGIWAVLAWLSILAHKNKDNLNGGNLVTVEDIVKQHWATYGRHYYTRYDYENVDAGAAKELMAHLVKLQSSISDVNTFIKGIRSDVANVASADEFEYKDPVDGSISKHQGIRYLFEDGSRLVFRLSGTGSEGATIRLYIEQYEKDSSKTGRDSQEALAPLVEVALKLSKMQEFTGRSAPTVIT

SEQ ID NO: 148 - DNA, 426 bp, Artificial Sequence
  pTEF1 promoter nucleotide sequence
gcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaggatcc

SEQ ID NO: 149 - DNA, 984 bp, Artificial Sequence
  pPGK1 promoter nucleotide sequence
ggaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaa

SEQ ID NO: 150 - DNA, 666 bp, Artificial Sequence
  pTDH3 promoter nucleotide sequence
cattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 151 - DNA, 565 bp, Artificial Sequence
  pTEF2 promoter nucleotide sequence
gatctgggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaaca

SEQ ID NO: 152 - DNA, 532 bp, Artificial Sequence
  pTPI1 promoter nucleotide sequence
agttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaa

SEQ ID NO: 153 - DNA, 805 bp, Artificial Sequence
  pPDC1 promoter nucleotide sequence
gatctatgcgactgggtgagcatatgttccgctgatgtgatgtgcaagataaacaagcaaggcagaaactaacttcttcttcatgtaataaacacaccccgcgtttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatctatcaaaa

SEQ ID NO: 154 - DNA, 192 bp, Artificial Sequence
  tCYC1 terminator nucleotide sequence
atccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaag

SEQ ID NO: 155 - DNA, 195 bp, Artificial Sequence
  tADH1 terminator nucleotide sequence
gtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctc

SEQ ID NO: 156 - DNA, 4611 bp, Saccharomyces cerevisiae
atgaatagatcattactgctacgtttgtcggataccggtgaacccattacaagctgctcttacggaaaaggtgtcttgacgctaccaccaattccgctccctaaggacgccccaaaggaccaaccgctctatacggtcaagctactggtatctgcaggttcccctgtcgctagggatgggctagtttggactaattgcccaccagatcacaacacgcccttcaagagggacaaattttacaaaaaaatcattcattccagctttcacgaggatgactgcattgacctgaatgtctacgctccaggctcgtactgcttttatctatctttcaggaacgataacgaaaaacttgagacaacaaggaaatactactttgttgccttgcccatgctttatataaacgatcagttcctacctttgaattccatcgctttacaaagtgttgtatcgaaatggctgggctctgactgggagcccatcctatcgaaaattgccgctaaaaactacaatatggtacatttcacccctctacaggaaagaggcgagtctaactcgccttactctatatacgaccaattgcagttcgaccaggaacactttaagtctcctgaagacgtgaaaaatttagttgagcatatacatcgcgatttaaacatgctttcattaacagatattgtttttaaccacacagctaataattctccttggttagttgagcacccggaggctgggtataaccacatcactgcgccacatctaatcagcgccatagagctcgaccaagaattgctcaattttagtaggaatttgaaatcctggggctatcctaccgaactgaaaaatatagaagatctcttcaagatcatggacggtattaaagtgcatgttttagggtcgttgaaactgtgggaatattatgcggtaaacgtgcaaacagctcttcgggatatcaaagcccattggaatgacgaatctaacgaaagttacagttttcccgagaatattaaagacatctcgtccgatttcgtaaaactagcttcctttgtgaaggacaacgtcactgagcctaacttcggcactcttggtgaaagaaactcaaacaggattaacgtgccaaaatttattcaactactgaagctcattaacgatggtggtagtgatgacagtgaatcttcgttggccacggctcaaaacatcttgaacgaggtcaacttacccttatatagagaatacgacgatgatgtcagtgagatactcgagcaactgttcaatcgtatcaaatatttgagattagatgacggtgggcccaagcaaggtccagtgaccgttgacgtgcccttaacagagccttattttacgaggttcaaaggaaaagatggtactgattatgccctcgccaacaatggctggatatggaatggtaacccactagtggattttgcatcgcagaattcaagagcttatttacgtagagaagttatcgtgtggggggactgtgtcaagttaagatacggtaaaagccctgaagactctccgtatctgtgggaaagaatgtccaagtatatagaaatgaacgccaagatatttgacgggttcagaattgacaactgccattctactccaatacatgttggcgaatatttcctagatttggcaagaaaatacaacccgaacctatatgtcgttgcagagctgttttctggttccgaaacactagattgtctgtttgttgaacggttgggtatctcctctttaatcagagaggcaatgcaagcctggtccgaagaagagttgtctagattagtccataagcatggcgggaggcccattggctcctataagtttgttcctatggatgacttctcatatcctgcggatattaatttaaacgaggagcattgtttcaacgactccaacgataactccataagatgtgtatcagagatcatgattccaaagattttaaccgccactccgccacacgctttattcatggactgtacccatgataatgaaactccctttgaaaaaagaacagtggaggatactttgcccaatgctgcattggtggctctttgctcgtccgccattggatctgtttatggctacgacgaaatttttccacatttactgaatttggtcactgaaaaaagacattatgacatttctacgcctactggtagcccctcgataggaataaccaaagtcaaggccactttgaattcgattagaacgagtataggagaaaaggcgtatgacattgaagactcagaaatgcatgtgcatcaccagggccagtacattacttttcatcgtatggatgttaaatccggaaaaggttggtacttgatagcaaggatgaaattttctgacaatgatgaccctaacgagactttaccaccagtggtgttaaaccaatccacctgttctctcaggttttcgtatgctttggaaagagttggcgatgaaattcccaacgacgataaattcattaaaggtattcccacgaaattaaaggagcttgaagggtttgacatttcttatgatgattctaagaagatttcaacgataaaactgcccaatgaattccctcaaggatctattgccatttttgagacccaacagaatggtgtggacgaatccttagatcattttataaggtcaggtgctttaaaggccacttcaagtttgactctagagtcaataaattccgtcttgtatcgtagtgagccggaagaatacgatgttagcgccggcgaaggtggtgcttatattattcctaattttggaaagcctgtgtattgtggtctgcaaggttgggtttccgtattaagaaaaattgtgttttacaatgatttagcacatcccctcagtgcaaatttaagaaatggacattgggctttagactacactatcagtagacttaattactatagcgatgaagcaggaatcaatgaagtgcagaactggctgcgttcaaggtttgatagagtgaaaaagttaccgagctacttagtgcccagttatttcgccttaattatcggcatcctctatggttgttgtcgcttaaaagcaatacagctaatgtcccgtaatattggtaaatctacattgtttgtacaaagcttatctatgacatcaatccagatggtttccagaatgaagtcaacctctattttaccaggcgaaaatgttccatctatggctgcagggttgccacactttagcgtaaactacatgagatgttgggggagagatgtattcatatcgctaagaggtatgctattaacaacaggtagatttgatgaagctaaagctcatatactagcctttgcaaagactttgaagcatggtttaattccaaacttgctggatgccggtagaaacccgagatataatgctcgtgatgctgcctggttcttcttgcaagctgtacaggattatgtttatattgttcctgatggcgaaaaaatattacaagagcaagtaacaaggagattcccactggatgatacttacattcctgtagatgatccaagggcatttagttactctagtaccttggaggagatcatttatgaaattttgagtaggcatgccaagggaattaaattcagagaggctaatgcaggtccaaatttagatcgtgttatgactgataaagggtttaatgttgaaattcatgtcgattggtcgactggcttaattcatggtggatctcagtataactgtggtacttggatggataagatgggtgaaagtgaaaaagcagggtctgttggtattcctggaacacccagagatggagccgcaatagaaatcaatgggcttttaaaaagtgctttaaggtttgttattgaactaaaaaacaagggattgtttaagttttccgatgtggagacgcaggacggcgggaggatcgatttcactgaatggaatcaattacttcaagacaatttcgaaaaaagatattatgttccggaggatccatcacaggatgcagattatgacgtgagcgctaaattgggtgttaatagacgggggatatacagagatttgtacaaatcaggaaagccttatgaagattatcagttaagaccaaattttgctattgccatgactgtggcaccagagttatttgtgcctgagcatgccataaaagcaatcaccattgcagatgaagtcttaagaggtccagtaggtatgcgtactttagacccaagcgattacaattaccgtccgtactacaacaacggagaagattcggatgattttgccacctcaaagggtagaaactatcaccaaggccctgagtgggtctggctttacggctactttttaagagcgttccatcatttccactttaaaaccagtccacgttgtcagaatgctgccaaagagaaaccatcctcttatttgtatcaacaattatactacagattaaaaggccatagaaaatggatttttgaaagtgtgtgggcaggattgacagagctaaccaataaagatggtgaagtatgcaatgactcaagccccacgcaagcctggagttctgcttgtttgttagatctattttatgatttatgggatgcctacgaagatgattcctga

SEQ ID NO: 157 - protein, 1536 aa, Saccharomyces cerevisiae
MNRSLLLRLSDTGEPITSCSYGKGVLTLPPIPLPKDAPKDQPLYTVKLLVSAGSPVARDGLVWTNCPPDHNTPFKRDKFYKKIIHSSFHEDDCIDLNVYAPGSYCFYLSFRNDNEKLETTRKYYFVALPMLYINDQFLPLNSIALQSVVSKWLGSDWEPILSKIAAKNYNMVHFTPLQERGESNSPYSIYDQLQFDQEHFKSPEDVKNLVEHIHRDLNMLSLTDIVFNHTANNSPWLVEHPEAGYNHITAPHLISAIELDQELLNFSRNLKSWGYPTELKNIEDLFKIMDGIKVHVLGSLKLWEYYAVNVQTALRDIKAHWNDESNESYSFPENIKDISSDFVKLASFVKDNVTEPNFGTLGERNSNRINVPKFIQLLKLINDGGSDDSESSLATAQNILNEVNLPLYREYDDDVSEILEQLFNRIKYLRLDDGGPKQGPVTVDVPLTEPYFTRFKGKDGTDYALANNGWIWNGNPLVDFASQNSRAYLRREVIVWGDCVKLRYGKSPEDSPYLWERMSKYIEMNAKIFDGFRIDNCHSTPIHVGEYFLDLARKYNPNLYVVAELFSGSETLDCLFVERLGISSLIREAMQAWSEEELSRLVHKHGGRPIGSYKFVPMDDFSYPADINLNEEHCFNDSNDNSIRCVSEIMIPKILTATPPHALFMDCTHDNETPFEKRTVEDTLPNAALVALCSSAIGSVYGYDEIFPHLLNLVTEKRHYDISTPTGSPSIGITKVKATLNSIRTSIGEKAYDIEDSEMHVHHQGQYITFHRMDVKSGKGWYLIARMKFSDNDDPNETLPPVVLNQSTCSLRFSYALERVGDEIPNDDKFIKGIPTKLKELEGFDISYDDSKKISTIKLPNEFPQGSIAIFETQQNGVDESLDHFIRSGALKATSSLTLESINSVLYRSEPEEYDVSAGEGGAYIIPNFGKPVYCGLQGWVSVLRKIVFYNDLAHPLSANLRNGHWALDYTISRLNYYSDEAGINEVQNWLRSRFDRVKKLPSYLVPSYFALIIGILYGCCRLKAIQLMSRNIGKSTLFVQSLSMTSIQMVSRMKSTSILPGENVPSMAAGLPHFSVNYMRCWGRDVFISLRGMLLTTGRFDEAKAHILAFAKTLKHGLIPNLLDAGRNPRYNARDAAWFFLQAVQDYVYIVPDGEKILQEQVTRRFPLDDTYIPVDDPRAFSYSSTLEEIIYEILSRHAKGIKFREANAGPNLDRVMTDKGFNVEIHVDWSTGLIHGGSQYNCGTWMDKMGESEKAGSVGIPGTPRDGAAIEINGLLKSALRFVIELKNKGLFKFSDVETQDGGRIDFTEWNQLLQDNFEKRYYVPEDPSQDADYDVSAKLGVNRRGIYRDLYKSGKPYEDYQLRPNFAIAMTVAPELFVPEHAIKAITIADEVLRGPVGMRTLDPSDYNYRPYYNNGEDSDDFATSKGRNYHQGPEWVWLYGYFLRAFHHFHFKTSPRCQNAAKEKPSSYLYQQLYYRLKGHRKWIFESVWAGLTELTNKDGEVCNDSSPTQAWSSACLLDLFYDLWDAYEDDS

SEQ ID NO: 158 - DNA, 2709 bp, Saccharomyces cerevisiae
atgccgccagctagtactagtactaccaatgatatgataaccgaagaacctacttctccacaccaaatcccaaggcttacaaggagacttacggggtttcttccccaagaaatcaagtcaattgacacgatgattcctttaaagtcaagagcgttatggaataagcatcaagtcaaaaaatttaacaaggcagaagattttcaagatagattcattgaccatgtggaaactacattagcacgttccctatataattgtgatgacatggctgcttatgaagctgcttcgatgagtattcgtgacaatttggtcattgactggaacaaaactcagcagaaattcaccacaagagacccaaagagagtttactatttgtctttggagtttttgatgggtagggctttggataatgccctgattaatatgaagattgaagatccggaagaccctgctgcctcaaagggaaaaccaagagaaatgattaaaggggctttggatgatttaggtttcaagttagaggatgtcttggaccaagaaccggacgcaggtttaggtaatggtggtctaggtcgtcttgcagcttgcttcgtcgactcaatggcaacggaaggcatccctgcctggggttatggtctacgttatgagtatggtatctttgctcaaaagattattgacggttaccaggtggaaactccagattactggttaaattctggtaatccatgggaaattgaacgtaacgaagtgcaaattcctgtcaccttttatggttatgttgatagaccagaaggcggtaaaactacactgagtgcgtcacaatggatcggtggggaaagagttcttgctgtcgcgtatgatttcccagttccgggtttcaagacttccaatgtaaataacttaagactatggcaagcaaggccaacaacagaatttgattttgcaaaattcaataatggtgactataaaaactctgtggctcagcaacaacgcgcagagtctataaccgctgtgttgtatccaaacgataactttgctcaaggtaaggagttgaggttgaaacagcagtacttctggtgtgctgcatccttacacgacatcttaagaagattcaaaaaatccaagaggccatggactgaatttcctgaccaagtggctattcagttgaatgatactcatccaactttagccatcgttgaattacagagagttttggtcgatctagaaaaactagattggcacgaggcttgggacatcgtgaccaagacttttgcttatactaaccacactgttatgcaagaggccctggaaaaatggcccgtcggcctctttggccatttgctacccagacatttggaaattatatatgatatcaactggttcttcttgcaagatgtggccaaaaaattccccaaggatgttgatcttttgtctcgtatatccatcatcgaagaaaactctccagaaagacagatcagaatggcctttttggctattgttggttcacacaaggttaatggtgttgctgaattgcactctgaattaatcaaaacgaccatatttaaagattttgtcaagttctatggtccatcaaagtttgtcaatgtcactaacggtatcacaccaaggagatggttgaagcaagctaacccttcattggctaaactgatcagtgaaacccttaacgatccaacagaggagtatttgttggacatggccaaactgacccagttgggaaaatatgttgaagataaggagtttttgaaaaaatggaaccaagtcaagcttaataataagatcagattagtagatttaatcaaaaaggaaaatgatggagtagacatcattaacagagagtatttggacgacaccttgtttgatatgcaagttaaacgtattcatgaatataagcgtcaacagctaaacgtctttggtattatataccgttacctggcaatgaagaatatgctgaagaacggtgcttcgatcgaagaagttgccaagaaatatccacgcaaggtttcaatctttggtggtaagagtgctcctggttactacatggctaagctgatcataaaattgatcaactgtgttgctgacattgttaataacgacgagtcaattgagcatttgttgaaggttgtctttgttgctgattataatgtttctaaggctgaaatcattattccagcaagtgacttgagtgagcatatttctactgctggtactgaagcgtctggtacttctaatatgaagtttgttatgaacggtggtttgattattggtactgttgatggtgccaatgtggaaatcacaagggaaattggtgaagataatgtcttcttgtttggtaacctaagtgaaaatgtcgaagaattgagatacaaccatcaataccatccacaagatttaccatctagtttggattctgttttatcctacattgaaagtggacaattttctccagaaaatccaaatgaattcaaacctttagtcgacagtattaagtaccacggcgattattacctggtcagtgatgactttgaatcctatctggccacccatgaattagtggaccaggagttccacaatcaaaggtcagaatggttaaaaaagagtgtcctgagcgttgcaaacgtcggcttctttagcagtgatcgttgtatcgaggaatactccgataccatttggaacgttgaaccagtgacttag

SEQ ID NO: 159 - protein, 902 aa, Saccharomyces cerevisiae
MPPASTSTTNDMITEEPTSPHQIPRLTRRLTGFLPQEIKSIDTMIPLKSRALWNKHQVKKFNKAEDFQDRFIDHVETTLARSLYNCDDMAAYEAASMSIRDNLVIDWNKTQQKFTTRDPKRVYYLSLEFLMGRALDNALINMKIEDPEDPAASKGKPREMIKGALDDLGFKLEDVLDQEPDAGLGNGGLGRLAACFVDSMATEGIPAWGYGLRYEYGIFAQKIIDGYQVETPDYWLNSGNPWEIERNEVQIPVTFYGYVDRPEGGKTTLSASQWIGGERVLAVAYDFPVPGFKTSNVNNLRLWQARPTTEFDFAKFNNGDYKNSVAQQQRAESITAVLYPNDNFAQGKELRLKQQYFWCAASLHDILRRFKKSKRPWTEFPDQVAIQLNDTHPTLAIVELQRVLVDLEKLDWHEAWDIVTKTFAYTNHTVMQEALEKWPVGLFGHLLPRHLEIIYDINWFFLQDVAKKFPKDVDLLSRISIIEENSPERQIRMAFLAIVGSHKVNGVAELHSELIKTTIFKDFVKFYGPSKFVNVTNGITPRRWLKQANPSLAKLISETLNDPTEEYLLDMAKLTQLGKYVEDKEFLKKWNQVKLNNKIRLVDLIKKENDGVDIINREYLDDTLFDMQVKRIHEYKRQQLNVFGIIYRYLAMKNMLKNGASIEEVAKKYPRKVSIFGGKSAPGYYMAKLIIKLINCVADIVNNDESIEHLLKVVFVADYNVSKAEIIIPASDLSEHISTAGTEASGTSNMKFVMNGGLIIGTVDGANVEITREIGEDNVFLFGNLSENVEELRYNHQYHPQDLPSSLDSVLSYIESGQFSPENPNEFKPLVDSIKYHGDYYLVSDDFESYLATHELVDQEFHNQRSEWLKKSVLSVANVGFFSSDRCIEEYSDTIWNVEPVT

SEQ ID NO: 160 - protein, 617 aa, Saccharomyces cerevisiae
MSKQFSHTTNDRRSSIIYSTSVGKAGLFTPADYIPQESEENLIEGEEQEGSEEEPSYTGNDDETEREGEYHSLLDANNSRTLQQEAWQQGYDSHDRKRLLDEERDLLIDNKLLSQHGNGGGDIESHGHGQAIGPDEEERPAEIANTWESAIESGQKISTTFKRETQVITMNALPLIFTFILQNSLSLASIFSVAHLGTKELGGVTLGSMTANITGLAAIQGLCTCLGTLCAQAYGAKNYHLVGVLVQRCAVITILAFLPMMYVWFVWSEKILALMIPERELCALAANYLRVTAFGVPGFILFECGKRFLQCQGIFHASTIVLFVCAPLNALMNYLLVWNDKIGIGYLGAPLSVVINYWLMTLGLLIYAMTTKHKERPLKCWNGIIPKEQAFKNWRKMINLAIPGVVMVEAEFLGFEVLTIFASHLGTDALGAQSIVATIASLAYQVPFSISVSTSTRVANFIGASLYDSCMITCRVSLLLSFVCSSMNMFVICRYKEQIASLFSTESAVVKMVVDTLPLLAFMQLFDAFNASTAGCLRGQGRQKIGGYINLVAFYCLGVPMAYVLAFLYHLGVGGLWLGITSALVMMSVCQGYAVFHGDRRRILGAARKRNAETHTS

SEQ ID NO: 161 - DNA, 331 bp, Saccharomyces cerevisiae
atgtctaaacaatttagtcataccaccaacgacagaagatcatcgattatctactccaccagtgtcggaaaggcagggcttttcacgcctgcagactacatcccacaggagtcagaagaaaacttaattgagggcgaagagcaagagggtagcgaagaagaaccttcctataccggcaatgacgatgagacggagagggaaggtgaataccattcgttattagatgccaacaattcgcggacattgcaacaagaagcgtggcaacaaggttatgactctcacgaccgtaagcgtttgcttgacgaagaacgggacctgctaatagacaaca

(19) United States
(12) Patent Application Publication
(10) Pub. No.: US 2020/0291442 A1
(43) Pub. Date: Sep. 17, 2020

(54) PRODUCTION OF STEVIOL GLYCOSIDES IN RECOMBINANT HOSTS

(71) Applicant: EVOLVA SA, Reinach (CH)

(72) Inventors: Veronique Douchin, Frederiksberg (DK); Swee Chuang Lim Hallwyl, Vallensbaek Strand (DK)

(21) Appl. No.: 16/754,247

(22) PCT Filed: Dec. 5, 2018
(86) PCT No.: PCT/EP2018/083689

§ 371 Date: Apr. 7, 2020

Related U.S. Application Data
(60) Provisional application No. 62/594,612, filed on Dec. 5, 2017.

(51) Int. Cl. C12P 19/56; C12N 1/18; C12N 1/16

(52) U.S. Cl. CPC C12P 19/56; C12N 1/16; C12N 1/18

(57) ABSTRACT

The invention relates to recombinant microorganisms and methods for producing steviol glycosides and steviol glycoside precursors.

### BACKGROUND OF THE INVENTION

### Field of the Invention

This disclosure relates to recombinant production of steviol glycosides, glycosides of steviol precursors, and steviol glycoside precursors in recombinant hosts. In particular, this disclosure relates to production of steviol glycosides comprising steviol-13-O-glucoside (13-SMG), steviol-19-O-glucoside (19-SMG), steviol-1,2-bioside, steviol-1,3-bioside, 1,2-stevioside, 1,3-stevioside, rubusoside, Rebaudioside A (RebA), Rebaudioside B (RebB), Rebaudioside C (RebC), Rebaudioside D (RebD), Rebaudioside E (RebE), Rebaudioside F (RebF), Rebaudioside M (RebM), Rebaudioside Q (RebQ), Rebaudioside I (RebI), dulcoside A, mono-glycosylated ent-kaurenoic acids, di-glycosylated ent-kaurenoic acids, tri-glycosylated ent-kaurenoic acids, mono-glycosylated ent-kaurenols, di-glycosylated ent-kaurenols, tri-glycosylated ent-kaurenols, tri-glycosylated steviol glycosides, tetra-glycosylated steviol glycosides, penta-glycosylated steviol glycosides, hexa-glycosylated steviol glycosides, hepta-glycosylated steviol glycosides, or isomers thereof in recombinant hosts.

### Description of Related Art

Sweeteners are well known as ingredients used most commonly in the food, beverage, or confectionary industries. The sweetener can either be incorporated into a final food product during production or for stand-alone use, when appropriately diluted, as a tabletop sweetener or an at-home replacement for sugars in baking. Sweeteners include natural sweeteners such as sucrose, high fructose corn syrup, molasses, maple syrup, and honey and artificial sweeteners such as aspartame, saccharine, and sucralose. Stevia extract is a natural sweetener that can be isolated and extracted from a perennial shrub, Stevia rebaudiana. Stevia is commonly grown in South America and Asia for commercial production of Stevia extract. Stevia extract, purified to various degrees, is used commercially as a high intensity sweetener in foods and in blends or alone as a tabletop sweetener.

Chemical structures for several steviol glycosides are shown in FIG. 2, including the diterpene steviol and various steviol glycosides. Extracts of the Stevia plant generally comprise steviol glycosides that contribute to the sweet flavor, although the amount of each steviol glycoside often varies, inter alia, among different production batches.

Recovery and purification of steviol glycosides from the Stevia plant have proven to be labor intensive and inefficient. Moreover, steviol glycoside compositions obtained from a plant-derived Stevia extract generally contain Stevia plant-derived components that can contribute to off-flavors. As such, there remains a need for a recombinant production system that can accumulate high yields of desired steviol glycosides, such as Reb A, RebD, and/or RebM and produce steviol glycoside compositions that are enriched for a one or more desired steviol glycosides relative to a steviol glycoside composition of Stevia plant with a reduced level of Stevia plant-derived components relative to a steviol glycoside composition obtained from a plant-derived Stevia extract. There also remains a need for improved production of steviol glycosides in recombinant hosts for commercial uses. As well, there remains a need for increasing uridine diphosphate glucose (UDP-glucose) formation in recombinant hosts in order to produce higher yields of steviol glycosides, including Reb A, RebD, and/or RebM.

### SUMMARY OF THE INVENTION

It is against the above background that the present invention provides certain advantages over the prior art.

Although this invention as disclosed herein is not limited to specific advantages or functionalities (such for example, the ability to scale up production of a one or more steviol glycosides or glycosides of a steviol precursor, purify the one or more steviol glycosides or glycosides of the steviol precursor, and produce steviol glycoside compositions where the different proportions of the various steviol glycosides provide the advantage of having a reduced level of Stevia plant-derived components relative to a steviol glycoside composition obtained from a plant-derived Stevia extract), the invention provides a recombinant host cell capable of producing one or more steviol glycosides or a steviol glycoside composition in a cell culture, comprising:
(a) a recombinant gene encoding a polypeptide capable of debranching glycogen; and/or
(b) a recombinant gene encoding a polypeptide capable of synthesizing glucose-1-phosphate.

In one aspect of the recombinant host cells disclosed herein, the polypeptide capable of debranching glycogen is capable of 4-α-glucanotransferase activity and α-1,6-amyloglucosidase activity.

In one aspect, the recombinant host cells disclosed herein further comprise:
(c) a gene encoding a polypeptide capable of synthesizing uridine 5′-triphosphate (UTP) from uridine diphosphate (UDP);
(d) a gene encoding a polypeptide capable of converting glucose-6-phosphate to glucose-1-phosphate; and/or
(e) a gene encoding a polypeptide capable of synthesizing uridine diphosphate glucose (UDP-glucose) from UTP and glucose-1-phosphate.

In one aspect of the recombinant host cells disclosed herein:
(a) the polypeptide capable of debranching glycogen comprises a polypeptide having at least 60% sequence identity to the amino acid sequence set forth in SEQ ID NO:157;
(b) the polypeptide capable of synthesizing glucose-1-phosphate comprises a polypeptide having at least 55% sequence identity to the amino acid sequence set forth in SEQ ID NO:159;
(c) the polypeptide capable of synthesizing UTP from UDP comprises a polypeptide having at least 60% sequence identity to the amino acid sequence set forth in SEQ ID NO:123;
(d) the polypeptide capable of converting glucose-6-phosphate to glucose-1-phosphate comprises a polypeptide having at least 60% sequence identity to the amino acid sequence set forth in any one of SEQ ID NOs:2, 119, or 143 or a polypeptide having at least 55% sequence identity to the amino acid sequence set forth in any one of SEQ ID NOs:141, 145, or 147; and/or
(e) the polypeptide capable of synthesizing UDP-glucose from UTP and glucose-1-phosphate comprises a polypeptide having at least 60% sequence identity to the amino acid sequence set forth in any one of SEQ ID NOs:121 or 127, a polypeptide having at least 55% sequence identity to the amino acid sequence set forth in any one of SEQ ID NOs:125, 129, 133, 135, 137, or 139 or a polypeptide having at least 70% sequence identity to the amino acid sequence set forth in SEQ ID NO:131.

In one aspect, the recombinant host cells disclosed herein further comprise:
(a) a gene encoding a polypeptide capable of glycosylating the steviol or the steviol glycoside at its C-13 hydroxyl group thereof;
(b) a gene encoding a polypeptide capable of beta 1,3 glycosylation of the C3′ of the 13-O-glucose, 19-O-glucose, or both 13-O-glucose and 19-O-glucose of the steviol glycoside;
(c) a gene encoding a polypeptide capable of glycosylating the steviol or the steviol glycoside at its C-19 carboxyl group thereof;
(d) a gene encoding a polypeptide capable of beta 1,2 glycosylation of the C2′ of the 13-O-glucose, 19-O-glucose, or both 13-O-glucose and 19-O-glucose of the steviol glycoside;
(e) a gene encoding a polypeptide capable of synthesizing geranylgeranyl pyrophosphate (GGPP) from farnesyl diphosphate (FPP) and isopentenyl diphosphate (IPP);
(f) a gene encoding a polypeptide capable of synthesizing ent-copalyl diphosphate from GGPP;
(g) a gene encoding an a polypeptide capable of synthesizing ent-kaurene from ent-copalyl diphosphate;
(h) a gene encoding a polypeptide capable of synthesizing ent-kaurenoic acid from ent-kaurene;
(i) a gene encoding a polypeptide capable of reducing cytochrome P450 complex; and/or
(j) a gene encoding a polypeptide capable of synthesizing steviol from ent-kaurenoic acid;

wherein at least one of the genes is a recombinant gene.

In one aspect of the recombinant host cells disclosed herein:
(a) the polypeptide capable of glycosylating the steviol or the steviol glycoside at its C-13 hydroxyl group thereof comprises a polypeptide having at least 55% sequence identity to the amino acid sequence set forth in SEQ ID NO:7;
(b) the polypeptide capable of beta 1,3 glycosylation of the C3′ of the 13-O-glucose, 19-O-glucose, or both 13-O-glucose and 19-O-glucose of the steviol glycoside comprises a polypeptide having at least 50% sequence identity to the amino acid sequence set forth in SEQ ID NO:9;
(c) the polypeptide capable of glycosylating the steviol or the steviol glycoside at its C-19 carboxyl group thereof comprises a polypeptide having at least 55% sequence identity to the amino acid sequence set forth in SEQ ID NO:4;
(d) the polypeptide capable of beta 1,2 glycosylation of the C2′ of the 13-O-glucose, 19-O-glucose, or both 13-O-glucose and 19-O-glucose of the steviol glycoside comprises a polypeptide having at least 80% sequence identity to the amino acid sequence set forth in SEQ ID NO:11; a polypeptide having at least 80% sequence identity to the amino acid sequence set forth in SEQ ID NO:13; or a polypeptide having at least 65% sequence identity to the amino acid sequence set forth in SEQ ID NO:16;
(e) the polypeptide capable of synthesizing GGPP comprises a polypeptide having at least 70% sequence identity to the amino acid sequence set forth in any one of SEQ ID NOs:20, 22, 24, 26, 28, 30, 32, or 116;
(f) the polypeptide capable of synthesizing ent-copalyl diphosphate comprises a polypeptide having at least 70% sequence identity to the amino acid sequence set forth in any one of SEQ ID NOs:34, 36, 38, 40, 42, or 120;
(g) the polypeptide capable of synthesizing ent-kaurene comprises a polypeptide having at least 70% sequence identity to the amino acid sequence set forth in any one of SEQ ID NOs:44, 46, 48, 50, or 52;
(h) the polypeptide capable of synthesizing ent-kaurenoic acid comprises a polypeptide having at least 70% sequence identity to the amino acid sequence set forth in any one of SEQ ID NOs:60, 62, 66, 68, 70, 72, 74, 76, or 117;
(i) the polypeptide capable of reducing cytochrome P450 complex comprises a polypeptide having at least 70% sequence identity to the amino acid sequence set forth in any one of SEQ ID NOs:78, 80, 82, 84, 86, 88, 90, 92; and/or
(j) the polypeptide capable of synthesizing steviol comprises a polypeptide having at least 70% sequence identity to the amino acid sequence set forth in any one of SEQ ID NOs:94, 97, 100, 101, 102, 103, 104, 106, 108, 110, 112, or 114.

In one aspect, the recombinant host cells disclosed herein comprise:
(a) the gene encoding the polypeptide capable of debranching glycogen having at least 60% sequence identity to the amino acid sequence set forth in SEQ ID NO:157;
(b) the gene encoding the polypeptide capable of synthesizing glucose-1-phosphate having at least 55% sequence identity to the amino acid sequence set forth in SEQ ID NO:159;
(c) the gene encoding the polypeptide capable of synthesizing uridine 5′-triphosphate (UTP) from uridine diphosphate (UDP) having at least 60% sequence identity to the amino acid sequence set forth in SEQ ID NO:123;
(d) the gene encoding the polypeptide capable of converting glucose-6-phosphate to glucose-1-phosphate having at least 60% sequence identity to the amino acid sequences set forth in any one of SEQ ID NOs:2 or 119; and
(e) the gene encoding the polypeptide capable of synthesizing UDP-glucose from UTP and glucose-1-phosphate having at least 60% sequence identity to the amino acid sequence set forth in SEQ ID NO:121; and

one or more of:
(f) the gene encoding the polypeptide capable of glycosylating the steviol or the steviol glycoside at its C-13 hydroxyl group thereof having at least 55% sequence identity to the amino acid sequence set forth in SEQ ID NO:7;
(g) the gene encoding the polypeptide capable of beta 1,3 glycosylation of the C3′ of the 13-O-glucose, 19-O-glucose, or both 13-O-glucose and 19-O-glucose of the steviol glycoside having at least 50% sequence identity to the amino acid sequence set forth in SEQ ID NO:9;
(h) the gene encoding the polypeptide capable of glycosylating the steviol or the steviol glycoside at its C-19 carboxyl group thereof having at least 55% sequence identity to the amino acid sequence set forth in SEQ ID NO:4;
(i) the gene encoding the polypeptide capable of beta 1,2 glycosylation of the C2′ of the 13-O-glucose, 19-O-glucose, or both 13-O-glucose and 19-O-glucose of the steviol glycoside comprises the polypeptide having at least 80% sequence identity to the amino acid sequence set forth in SEQ ID NO:11; the polypeptide having at least 80% sequence identity to the amino acid sequence set forth in SEQ ID NO:13; or the polypeptide having at least 65% sequence identity to the amino acid sequence set forth in SEQ ID NO:16;

wherein at least one of the genes is a recombinant gene.

In one aspect, the recombinant host cells disclosed herein comprise:
(a) the recombinant gene encoding the polypeptide capable of debranching glycogen having at least 60% sequence identity to the amino acid sequence set forth in SEQ ID NO:157; and/or
(b) the recombinant gene encoding the polypeptide capable of synthesizing glucose-1-phosphate having at least 55% sequence identity to the amino acid sequence set forth in SEQ ID NO:159;
wherein the recombinant gene encoding the polypeptide capable of debranching glycogen and/or the recombinant gene encoding the polypeptide capable of synthesizing glucose-1-phosphate are overexpressed relative to a corresponding host cell lacking the one or more recombinant genes.

In one aspect of the recombinant host cells disclosed herein, the gene encoding the polypeptide capable of debranching glycogen and/or the gene encoding the polypeptide capable of synthesizing glucose-1-phosphate are overexpressed by at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% relative to a corresponding host cell lacking the one or more recombinant genes.

In one aspect of the recombinant host cells disclosed herein, expression of the one or more recombinant genes increase the amount of UDP-glucose accumulated by the cell relative to a corresponding host lacking the one or more recombinant genes.

In one aspect of the recombinant host cells disclosed herein, the expression of the one or more recombinant genes increases the amount of UDP-glucose accumulated by the cell by at least 10%, at least 25%, or at least 50%, at least 100%, at least 150%, at least 200%, or at least 250% relative to a corresponding host lacking the one or more recombinant genes.

In one aspect of the recombinant host cells disclosed herein, the expression of the one or more recombinant genes increases an amount of the one or more steviol glycosides or the steviol glycoside composition produced by the cell relative to a corresponding host lacking the one or more recombinant genes.

In one aspect of the recombinant host cells disclosed herein, the expression of the one or more recombinant genes increases the amount of the one or more steviol glycosides produced by the cell by at least 5%, or at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% relative to a corresponding host cell lacking the one or more recombinant genes.

In one aspect of the recombinant host cells disclosed herein, the expression of the one or more recombinant genes increases an amount of RebA, RebD, and/or RebM produced by the cell by at least 5%, or at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% relative to a corresponding host cell lacking the one or more recombinant genes.

In one aspect of the recombinant host cells disclosed herein, the expression of the one or more recombinant genes decreases the amount of the one of one or more steviol glycosides or the steviol glycoside composition accumulated by the cell relative to a corresponding host lacking the one or more recombinant genes.

In one aspect of the recombinant host cells disclosed herein, the expression of the one or more recombinant genes decreases the amount of the one or more steviol glycosides accumulated by the cell by at least 5%, or at least 10%, or at least 15%, or at least 20%, or at least 30%, or at least 40%, or at least 50% relative to a corresponding host cell lacking the one or more recombinant genes relative to a corresponding host lacking the one or more recombinant genes.

In one aspect of the recombinant host cells disclosed herein, the expression of the one or more recombinant genes decreases an amount of 13-SMG accumulated by the cell relative to a corresponding host lacking the one or more recombinant genes.

In one aspect of the recombinant host cells disclosed herein, the expression of the one or more recombinant genes increases the amount of total steviol glycosides produced by the cell by at least 5%, or at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% relative to a corresponding host lacking the one or more recombinant genes.

In one aspect of the recombinant host cells disclosed herein, the expression of the one or more recombinant genes decreases the amount of total steviol glycosides produced by the cell by less than 10%, or less than 5%, or less than 2.5% relative to a corresponding host lacking the one or more recombinant genes.

In one aspect of the recombinant host cells disclosed herein, the one or more steviol glycosides is, or the steviol glycoside composition comprises, steviol-13-O-glucoside (13-SMG), steviol-1,2-Bioside, steviol-1,3-Bioside, steviol-19-O-glucoside (19-SMG), 1,2-Stevioside, 1,3-stevioside (RebG), rubusoside, rebaudioside A (RebA), rebaudioside B (RebB), rebaudioside C (RebC), rebaudioside D (RebD), rebaudioside E (RebE), rebaudioside F (RebF), rebaudioside M (RebM), rebaudioside Q (RebQ), rebaudioside I (RebI), dulcoside A, and/or an isomer thereof.

In one aspect of the recombinant host cells disclosed herein, the recombinant host cell is a plant cell, a mammalian cell, an insect cell, a fungal cell from Aspergillus genus or a yeast cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species, an algal cell or a bacterial cell from Escherichia coli species or Bacillus genus.

In one aspect of the recombinant host cells disclosed herein, the recombinant host cell is a Saccharomyces cerevisiae cell.

In one aspect of the recombinant host cells disclosed herein, the recombinant host cell is a Yarrowia lipolytica cell.

The invention also provides a method of producing one or more steviol glycosides or a steviol glycoside composition in a cell culture, comprising culturing the recombinant host cells disclosed herein in the cell culture, under conditions in which the genes are expressed, and wherein the one or more steviol glycosides or the steviol glycoside composition is produced by the recombinant host cell.

In one aspect of the methods disclosed herein, the genes are constitutively expressed.

In one aspect of the methods disclosed herein, the expression of the genes is induced.

In one aspect of the methods disclosed herein, the amount of RebA, RebD, and/or RebM produced by the cell is increased by at least 5%, or at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% relative to a corresponding host lacking the one or more recombinant genes.

In one aspect of the methods disclosed herein, the amount of 13-SMG accumulated by the cell is decreased by at least 10%, at least 25%, or at least 50% relative to a corresponding host lacking the one or more recombinant genes.

In one aspect of the methods disclosed herein, the amount of total steviol glycosides produced by the cell is increased by at least 5%, or at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% relative to a corresponding host lacking the one or more recombinant genes.

In one aspect of the methods disclosed herein, the amount of total steviol glycosides produced by the cell is decreased by less than 10%, or less than 5%, or less than 2.5% relative to a corresponding host lacking the one or more recombinant genes.

In one aspect of the methods disclosed herein, the recombinant host cell is grown in a fermentor at a temperature for a period of time, wherein the temperature and period of time facilitate the production of the one or more steviol glycosides or the steviol glycoside composition.

In one aspect of the methods disclosed herein, the amount of UDP-glucose accumulated by the cell by at least 10%, at least 25%, or at least 50%, at least 100%, at least 150%, at least 200%, or at least 250% relative to a corresponding host lacking the one or more recombinant genes.

In one aspect, the methods disclosed herein further comprise isolating the produced one or more steviol glycosides or the steviol glycoside composition from the cell culture.

In one aspect of the methods disclosed herein, the isolating step comprises separating a liquid phase of the cell culture from a solid phase of the cell culture to obtain a supernatant comprising the produced one or more steviol glycosides or the steviol glycoside composition, and:
(a) contacting the supernatant with one or more adsorbent resins in order to obtain at least a portion of the produced one or more steviol glycosides or the steviol glycoside composition; or
(b) contacting the supernatant with one or more ion exchange or reversed-phase chromatography columns in order to obtain at least a portion of the produced one or more steviol glycosides or the steviol glycoside composition; or
(c) crystallizing or extracting the produced one or more steviol glycosides or the steviol glycoside composition;

thereby isolating the produced one or more steviol glycosides or the steviol glycoside composition.

In one aspect, the methods disclosed herein further comprise recovering the one or more steviol glycosides or the steviol glycoside composition from the cell culture.

In one aspect of the methods disclosed herein, the recovered one or more steviol glycosides or the steviol glycoside composition is enriched for the one or more steviol glycosides relative to a steviol glycoside composition of Stevia plant and has a reduced level of Stevia plant-derived components relative to a steviol glycoside composition obtained from a plant-derived Stevia extract.

The invention also provides a method for producing one or more steviol glycosides or a steviol glycoside composition, comprising whole-cell bioconversion of a plant-derived or synthetic steviol and/or steviol glycosides in a cell culture of a recombinant host cell using:
(a) a polypeptide capable of debranching glycogen, comprising a polypeptide having at least 60% sequence identity to the amino acid sequence set forth in SEQ ID NO:157; and/or
(b) a polypeptide capable of synthesizing glucose-1-phosphate, comprising a polypeptide having at least 55% sequence identity to the amino acid sequence set forth in SEQ ID NO:159; and

optionally, one or more of:
(c) a polypeptide capable of synthesizing UTP from UDP, comprising a polypeptide having at least 60% sequence identity to the amino acid sequence set forth in SEQ ID NO:123;
(d) a polypeptide capable of converting glucose-6-phosphate to glucose-1-phosphate, comprising a polypeptide having at least 60% sequence identity to the amino acid sequence set forth in any one of SEQ ID NO:2, 119, or 143; or at least 55% sequence identity to the amino acid sequence set forth in any one of SEQ ID NOs:141, 145, or 147; and/or
(e) a polypeptide capable of synthesizing UDP-glucose from UTP and glucose-1-phosphate, comprising a polypeptide having at least 60% sequence identity to the amino acid sequence set forth in any one of SEQ ID NO:121 or 127; at least 55% sequence identity to the amino acid sequence set forth in any one of SEQ ID NOs:125, 129, 133, 135, 137, or 139; or at least 70% sequence identity to the amino acid sequence set forth in SEQ ID NO:131, and

one or more of:
(f) a polypeptide capable of glycosylating a steviol or a steviol glycoside at its C-13 hydroxyl group thereof;
(g) a polypeptide capable of beta 1,3 glycosylation of the C3′ of the 13-O-glucose, 19-O-glucose, or both 13-O-glucose and 19-O-glucose of the steviol glycoside;
(h) a polypeptide capable of glycosylating the steviol or the steviol glycoside at its C-19 carboxyl group thereof; and/or
(i) a polypeptide capable of beta 1,2 glycosylation of the C2′ of the 13-O-glucose, 19-O-glucose, or both 13-O-glucose and 19-O-glucose of a steviol glycoside;

wherein at least one of the polypeptides is a recombinant polypeptide expressed in the recombinant host cell; and producing the one or more steviol glycosides or the steviol glycoside composition thereby.

In one aspect of the methods disclosed herein:
(f) the polypeptide capable of glycosylating the steviol or the steviol glycoside at its C-13 hydroxyl group thereof comprises a polypeptide having at least 55% sequence identity to the amino acid sequence set forth in SEQ ID NO:7;
(g) the polypeptide capable of beta 1,3 glycosylation of the C3′ of the 13-O-glucose, 19-O-glucose, or both 13-O-glucose and 19-O-glucose of the steviol glycoside comprises a polypeptide having at least 50% sequence identity to the amino acid sequence set forth in SEQ ID NO:9;
(h) the polypeptide capable of glycosylating the steviol or the steviol glycoside at its C-19 carboxyl group thereof comprises a polypeptide having at least 55% sequence identity to the amino acid sequence set forth in SEQ ID NO:4;
(i) the polypeptide capable of beta 1,2 glycosylation of the C2′ of the 13-O-glucose, 19-O-glucose, or both 13-O-glucose and 19-O-glucose of the steviol glycoside comprises a polypeptide having at least 80% sequence identity to the amino acid sequence set forth in SEQ ID NO:11; a polypeptide having at least 80% sequence identity to the amino acid sequence set forth in SEQ ID NO:13; or a polypeptide having at least 65% sequence identity to the amino acid sequence set forth in SEQ ID NO:16.

In one aspect of the methods disclosed herein, the recombinant host cell is a plant cell, a mammalian cell, an insect cell, a fungal cell from Aspergillus genus or a yeast cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species, an algal cell or a bacterial cell from Escherichia coli species or Bacillus genus.

In one aspect of the methods disclosed herein, the recombinant host cell is a Saccharomyces cerevisiae cell.

In one aspect of the methods disclosed herein, the recombinant host cell is a Yarrowia lipolytica cell.

In one aspect of the methods disclosed herein, the one or more steviol glycosides is, or the steviol glycoside composition comprises, steviol-13-O-glucoside (13-SMG), steviol-1,2-Bioside, steviol-1,3-Bioside, steviol-19-O-glucoside (19-SMG), 1,2-stevioside, 1,3-stevioside (RebG), rubusoside, rebaudioside A (RebA), rebaudioside B (RebB), rebaudioside C (RebC), rebaudioside D (RebD), rebaudioside E (RebE), rebaudioside F (RebF), rebaudioside M (RebM), rebaudioside Q (RebQ), rebaudioside I (RebI), dulcoside A, and/or an isomer thereof.

The invention also provides a cell culture, comprising the recombinant host cells disclosed herein, the cell culture further comprising:
(a) the one or more steviol glycosides or the steviol glycoside composition produced by the recombinant host cell;
(b) glucose, fructose, sucrose, xylose, rhamnose, UDP-glucose, UDP-rhamnose, UDP-xylose, and/or N-acetyl-glucosamine; and
(c) supplemental nutrients comprising trace metals, vitamins, salts, YNB, and/or amino acids;

wherein the one or more steviol glycosides or the steviol glycoside composition is present at a concentration of at least 1 mg/liter of the cell culture;

wherein the cell culture is enriched for the one or more steviol glycosides or the steviol glycoside composition relative to a steviol glycoside composition from a Stevia plant and has a reduced level of Stevia plant-derived components relative to a plant-derived Stevia extract.

The invention also provides a cell culture, comprising the recombinant host cells disclosed herein, the cell culture further comprising:
(a) the one or more steviol glycosides or the steviol glycoside composition produced by the recombinant host cell;
(b) glucose, fructose, sucrose, xylose, rhamnose, UDP-glucose, UDP-rhamnose, UDP-xylose, and/or N-acetyl-glucosamine; and
(c) supplemental nutrients comprising trace metals, vitamins, salts, YNB, and/or amino acids;

wherein UDP-glucose is present in the cell culture at a concentration of at least 100 μM;

wherein the cell culture is enriched for UGP-glucose relative to a steviol glycoside composition from a Stevia plant and has a reduced level of Stevia plant-derived components relative to a plant-derived Stevia extract.

The invention also provides a cell lysate from the recombinant host cells disclosed herein grown in the cell culture, comprising:
(a) the one or more steviol glycosides or the steviol glycoside composition produced by the recombinant host cell;
(b) glucose, fructose, sucrose, xylose, rhamnose, UDP-glucose, UDP-rhamnose, UDP-xylose, and/or N-acetyl-glucosamine; and/or
(c) supplemental nutrients comprising trace metals, vitamins, salts, yeast nitrogen base, YNB, and/or amino acids;

wherein the one or more steviol glycosides or the steviol glycoside composition produced by the recombinant host cell is present at a concentration of at least 1 mg/liter of the cell culture.

The invention also provides one or more steviol glycosides produced by the recombinant host cells disclosed herein;

wherein the one or more steviol glycosides produced by the recombinant host cell are present in relative amounts that are different from a steviol glycoside composition from a Stevia plant and have a reduced level of Stevia plant-derived components relative to a plant-derived Stevia extract.

The invention also provides one or more steviol glycosides produced by the methods disclosed herein;

wherein the one or more steviol glycosides produced by the recombinant host cell are present in relative amounts that are different from a steviol glycoside composition from a Stevia plant and have a reduced level of Stevia plant-derived components relative to a plant-derived Stevia extract.

The invention also provides a sweetener composition, comprising the one or more steviol glycosides disclosed herein.

The invention also provides a food product comprising, the sweetener composition disclosed herein.

The invention also provides a beverage or a beverage concentrate, comprising the sweetener composition disclosed herein.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows the biochemical pathway for producing steviol from geranylgeranyl diphosphate using geranylgeranyl diphosphate synthase (GGPPS), ent-copalyl diphosphate synthase (CDPS), ent-kaurene synthase (KS), ent-kaurene oxidase (KO), and ent-kaurenoic acid hydroxylase (KAH) polypeptides.

FIG. 2 shows representative primary steviol glycoside glycosylation reactions catalyzed by suitable UGT enzymes and chemical structures for several of the compounds found in Stevia extracts.

FIG. 3 shows representative reactions catalyzed by enzymes involved in the UDP-glucose biosynthetic pathway, including uracil permease (FUR4), uracil phosphoribosyltransferase (FUR1), orotate phosphoribosyltransferase 1 (URA5), orotate phosphoribosyltransferase 2 (URA10), orotidine 5′-phosphate decarboxylase (URA3), uridylate kinase (URA6), nucleoside diphosphate kinase (YNK1), phosphoglucomutase-1 (PGM1), phosphoglucomutase-2 (PGM2), UTP-glucose-1-phosphate uridylyltransferase (UGP1), glycogenin glucosyltransferase-1 (GLG1), glycogenin glucosyltransferase-2 (GLG-2), glycogen synthase-1 (GSY1), glycogen synthase-2 (GSY2), glycogen branching enzyme (GLC3), glycogen debranching enzyme (GDB1), and glycogen phosphorylase (GPH1). See, e.g., Daran et al., 1995, Eur. J. Biochem. 233(2):520-30; François and Parrou, 2001, FEMS Microbiol. Rev. 25(1):125-45.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Before describing the present invention in detail, a number of terms will be defined. As used herein, the singular forms “a,” “an,” and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof, in either single-stranded or double-stranded embodiments depending on context as understood by the skilled worker.

As used herein, the terms “microorganism,” “microorganism host,” and “microorganism host cell” can be used interchangeably. As used herein, the terms “recombinant host” and “recombinant host cell” can be used interchangeably. The person of ordinary skill in the art will appreciate that the terms “microorganism,” microorganism host,” and “microorganism host cell,” when used to describe a cell comprising a recombinant gene, may be taken to mean “recombinant host” or “recombinant host cell.” As used herein, the term “recombinant host” is intended to refer to a host, the genome of which has been augmented by at least one DNA sequence. Such DNA sequences include but are not limited to genes that are not naturally present, DNA sequences that are not normally transcribed into RNA or translated into a protein (“expressed”), and other genes or DNA sequences which one desires to introduce into a host. It will be appreciated that typically the genome of a recombinant host described herein is augmented through stable introduction of one or more recombinant genes. Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. In some aspects, the introduced DNA is introduced into the genome in a location different than where the corresponding endogenous DNA segment originally resided. Suitable recombinant hosts include microorganisms.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species or can be a DNA sequence that originated from or is present in the same species but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. In some aspects, said recombinant genes are encoded by cDNA. In other embodiments, recombinant genes are synthetic and/or codon-optimized for expression in S. cerevisiae.

As used herein, the term “engineered biosynthetic pathway” refers to a biosynthetic pathway that occurs in a recombinant host, as described herein. In some aspects, one or more steps of the biosynthetic pathway do not naturally occur in an unmodified host. In some embodiments, a heterologous version of a gene is introduced into a host that comprises an endogenous version of the gene.

As used herein, the term “endogenous” gene refers to a gene that originates from and is produced or synthesized within a particular organism, tissue, or cell. In some embodiments, the endogenous gene is a yeast gene. In some embodiments, the gene is endogenous to S. cerevisiae, including, but not limited to S. cerevisiae strain S288C. In some embodiments, an endogenous yeast gene is overexpressed. As used herein, the term “overexpress” is used to refer to the expression of a gene in an organism at levels higher than the level of gene expression in a wild type organism. See, e.g., Prelich, 2012, Genetics 190:841-54. See, e.g., Giaever & Nislow, 2014, Genetics 197(2):451-65. In some aspects, overexpression can be performed by integration using the USER cloning system; see, e.g., Nour-Eldin et al., 2010, Methods Mol Biol. 643:185-200. As used herein, the terms “deletion,” “deleted,” “knockout,” and “knocked out” can be used interchangeably to refer to an endogenous gene that has been manipulated to no longer be expressed in an organism, including, but not limited to, S. cerevisiae. In some aspects, the terms “deletion,” “deleted,” “knockout,” and “knocked out” can be used interchangeably to refer to an endogenous gene that has been mutated so that the endogenous gene has reduced activity or no activity.

As used herein, the terms “heterologous sequence” and “heterologous coding sequence” are used to describe a sequence derived from a species other than the recombinant host. In some embodiments, the recombinant host is an S. cerevisiae cell, and a heterologous sequence is derived from an organism other than S. cerevisiae. A heterologous coding sequence, for example, can be from a prokaryotic microorganism, a eukaryotic microorganism, a plant, an animal, an insect, or a fungus different than the recombinant host expressing the heterologous sequence. In some embodiments, a coding sequence is a sequence that is native to the host.

As used herein, the terms “heterologous sequence” and “heterologous coding sequence” are used to describe a sequence derived from a species other than the recombinant host. In some embodiments, the recombinant host is an S. cerevisiae cell, and a heterologous sequence is derived from an organism other than S. cerevisiae. A heterologous coding sequence, for example, can be from a prokaryotic microorganism, a eukaryotic microorganism, a plant, an animal, an insect, or a fungus different than the recombinant host expressing the heterologous sequence. In some embodiments, a coding sequence is a sequence that is native to the host.

As used herein, the term “constitutive,” “constitutive expression,” or “constitutively expressed” refers to a continuous transcription of a gene resulting in the continuous expression of a protein.

As used herein, the term “inducible,” “inducible expression,” or “inducibly expressed” refers to the expression of a gene in response to a stimuli. Stimuli include, but are not limited to, chemicals, stress, or biotic stimuli.

A “selectable marker” can be one of any number of genes that complement host cell auxotrophy, provide antibiotic resistance, or result in a color change. Linearized DNA fragments of the gene replacement vector then are introduced into the cells using methods well known in the art (see below). Integration of the linear fragments into the genome and the disruption of the gene can be determined based on the selection marker and can be verified by, for example, PCR or Southern blot analysis. Subsequent to its use in selection, a selectable marker can be removed from the genome of the host cell by, e.g., Cre-LoxP systems (see, e.g., Gossen et al., 2002, Ann. Rev. Genetics 36:153-173 and U.S. 2006/0014264). Alternatively, a gene replacement vector can be constructed in such a way as to include a portion of the gene to be disrupted, where the portion is devoid of any endogenous gene promoter sequence and encodes none, or an inactive fragment of, the coding sequence of the gene.

As used herein, the terms “variant” and “mutant” are used to describe a protein sequence that has been modified at one or more amino acids, compared to the wild-type sequence of a particular protein.

As used herein, the term “inactive fragment” is a fragment of the gene that encodes a protein having, e.g., less than 10% (e.g., less than 9%, less than 8%, less than 7%, less than 6%, less than 5%, less than 4%, less than 3%, less than 2%, less than 1%, or 0%) of the activity of the protein produced from the full-length coding sequence of the gene. Such a portion of a gene is inserted in a vector in such a way that no known promoter sequence is operably linked to the gene sequence, but that a stop codon and a transcription termination sequence are operably linked to the portion of the gene sequence. This vector can be subsequently linearized in the portion of the gene sequence and transformed into a cell. By way of single homologous recombination, this linearized vector is then integrated in the endogenous counterpart of the gene with inactivation thereof.

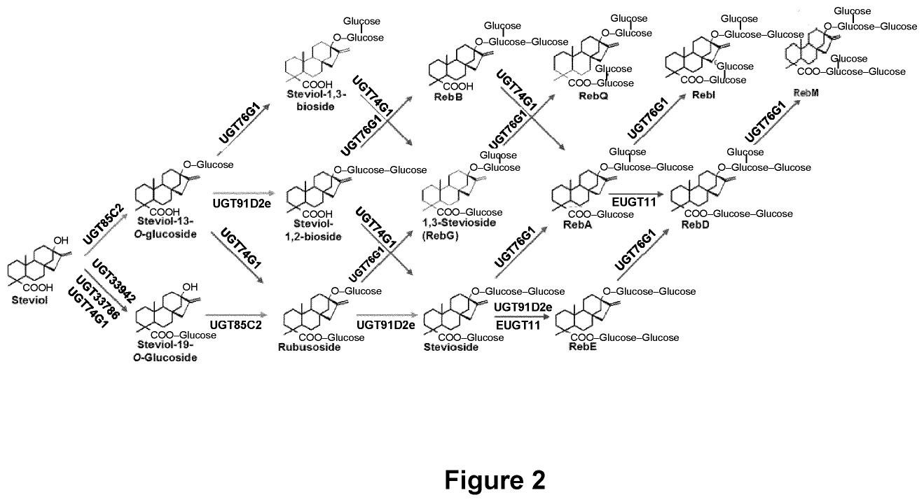

As used herein, the term “steviol glycoside” refers to rebaudioside A (RebA) (CAS #58543-16-1), rebaudioside B (RebB) (CAS #58543-17-2), rebaudioside C (RebC) (CAS #63550-99-2), rebaudioside D (RebD) (CAS #63279-13-0), rebaudioside E (RebE) (CAS #63279-14-1), rebaudioside F (RebF) (CAS #438045-89-7), rebaudioside M (RebM) (CAS #1220616-44-3), Rubusoside (CAS #63849-39-4), Dulcoside A (CAS #64432-06-0), rebaudioside I (RebI) (MassBank Record: FU000332), rebaudioside Q (RebQ), 1,2-Stevioside (CAS #57817-89-7), 1,3-Stevioside (RebG), Steviol-1,2-Bioside (MassBank Record: FU000299), Steviol-1,3-Bioside, Steviol-13-O-glucoside (13-SMG), Steviol-19-O-glucoside (19-SMG), a tri-glycosylated steviol glycoside, a tetra-glycosylated steviol glycoside, a penta-glycosylated steviol glycoside, a hexa-glycosylated steviol glycoside, a hepta-glycosylated steviol glycoside, and isomers thereof. See FIG. 2; see also, Steviol Glycosides Chemical and Technical Assessment 69th JECFA, 2007, prepared by Harriet Wallin, Food Agric. Org.

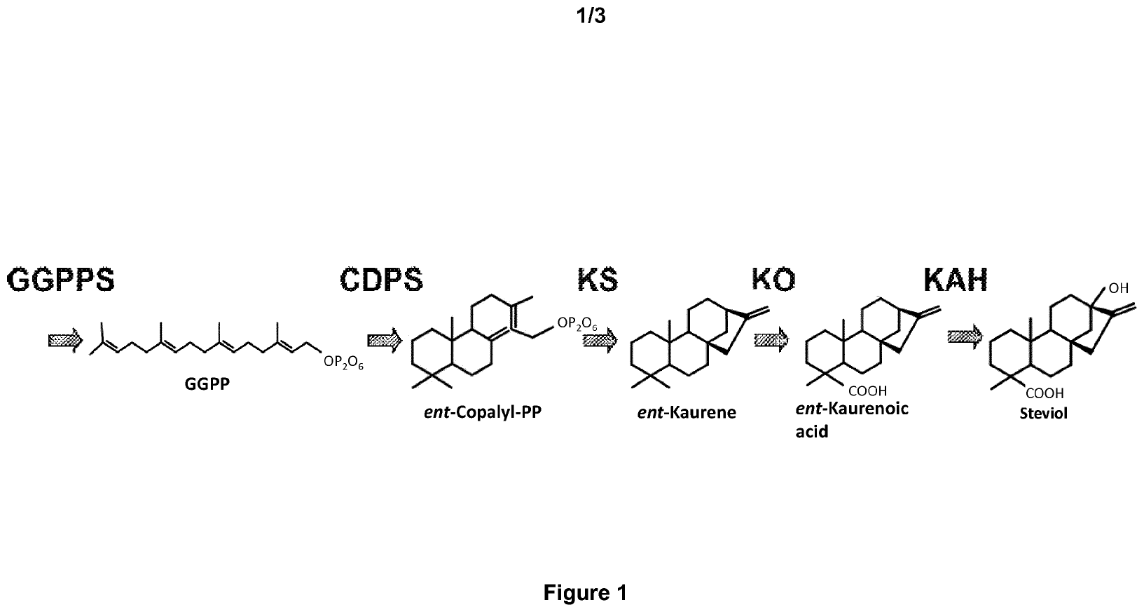

As used herein, the terms “steviol glycoside precursor” and “steviol glycoside precursor compound” are used to refer to intermediate compounds in the steviol glycoside biosynthetic pathway. Steviol glycoside precursors include, but are not limited to, geranylgeranyl diphosphate (GGPP), ent-copalyl-diphosphate, ent-kaurene, ent-kaurenol, ent-kaurenal, ent-kaurenoic acid, and steviol. See FIG. 1. In some embodiments, steviol glycoside precursors are themselves steviol glycoside compounds. For example, 19-SMG, rubusoside, 1,2-stevioside, and RebE are steviol glycoside precursors of RebM. See FIG. 2. Also as used herein, the terms “steviol precursor” and “steviol precursor compound” are used to refer to intermediate compounds in the steviol biosynthetic pathway. Steviol precursors may also be steviol glycoside precursors, and include, but are not limited to, geranylgeranyl diphosphate (GGPP), ent-copalyl-diphosphate, ent-kaurene, ent-kaurenol, ent-kaurenal, and ent-kaurenoic acid.

As used herein, the term “contact” is used to refer to any physical interaction between two objects. For example, the term “contact” may refer to the interaction between an enzyme and a substrate. In another example, the term “contact” may refer to the interaction between a liquid (e.g., a supernatant) and an adsorbent resin.

Steviol glycosides and/or steviol glycoside precursors can be produced in vivo (i.e., in a recombinant host), in vitro (i.e., enzymatically), or by whole cell bioconversion. As used herein, the terms “produce” and “accumulate” can be used interchangeably to describe synthesis of steviol glycosides and steviol glycoside precursors in vivo, in vitro, or by whole cell bioconversion.

As used herein, the terms “culture broth,” “culture medium,” and “growth medium” can be used interchangeably to refer to a liquid or solid that supports growth of a cell. A culture broth can comprise glucose, fructose, sucrose, trace metals, vitamins, salts, yeast nitrogen base (YNB), and/or amino acids. The trace metals can be divalent cations, including, but not limited to, Mn²⁺ and/or Mg²⁺. In some embodiments, Mn²⁺ can be in the form of MnCl₂ dihydrate and range from approximately 0.01 g/L to 100 g/L. In some embodiments, Mg²⁺ can be in the form of MgSO₄ heptahydrate and range from approximately 0.01 g/L to 100 g/L. For example, a culture broth can comprise i) approximately 0.02-0.03 g/L MnCl₂ dihydrate and approximately 0.5-3.8 g/L MgSO₄ heptahydrate, ii) approximately 0.03-0.06 g/L MnCl₂ dihydrate and approximately 0.5-3.8 g/L MgSO₄ heptahydrate, and/or iii) approximately 0.03-0.17 g/L MnCl₂ dihydrate and approximately 0.5-7.3 g/L MgSO₄ heptahydrate. Additionally, a culture broth can comprise one or more steviol glycosides produced by a recombinant host, as described herein.

Recombinant steviol glycoside-producing Saccharomyces cerevisiae (S. cerevisiae) strains are described in WO 2011/153378, WO 2013/022989, WO 2014/122227, and WO 2014/122328, each of which is incorporated by reference in their entirety. Methods of producing steviol glycosides in recombinant hosts, by whole cell bio-conversion, and in vitro are also described in WO 2011/153378, WO 2013/022989, WO 2014/122227, and WO 2014/122328.

In some embodiments, a recombinant host comprising a gene encoding a polypeptide capable of synthesizing geranylgeranyl pyrophosphate (GGPP) from farnesyl diphosphate (FPP) and isopentenyl diphosphate (IPP) (e.g., a geranylgeranyl diphosphate synthase (GGPPS) polypeptide); a gene encoding a polypeptide capable of synthesizing ent-copalyldiphosphate from GGPP (e.g., a ent-copalyl diphosphate synthase (CDPS) polypeptide); a gene encoding a polypeptide capable of synthesizing ent-kaurene from ent-copalyl diphosphate (e.g., a kaurene synthase (KS) polypeptide); a gene encoding a polypeptide capable of synthesizing ent-kaurenoic acid, ent-kaurenol, and/or ent-kaurenal from ent-kaurene (e.g., a kaurene oxidase (KO) polypeptide); a gene encoding a polypeptide capable of reducing cytochrome P450 complex (e.g., a cytochrome P450 reductase (CPR) polypeptide or a P450 oxidoreductase (POR) polypeptide; for example, but not limited to a polypeptide capable of electron transfer from NADPH to cytochrome P450 complex during conversion of NADPH to NADP⁺, which is utilized as a cofactor for terpenoid biosynthesis); a gene encoding a polypeptide capable of synthesizing steviol from ent-kaurenoic acid (e.g., a steviol synthase (KAH) polypeptide); and/or a gene encoding a bifunctional polypeptide capable of synthesizing ent-copalyl diphosphate from GGPP and synthesizing ent-kaurene from ent-copalyl diphosphate (e.g., an ent-copalyl diphosphate synthase (CDPS)—ent-kaurene synthase (KS) polypeptide) can produce steviol in vivo. See, e.g., FIG. 1. The skilled worker will appreciate that one or more of these genes can be endogenous to the host provided that at least one (and in some embodiments, all) of these genes is a recombinant gene introduced into the recombinant host.

In some embodiments, a recombinant host comprising a gene encoding a polypeptide capable of glycosylating a steviol or a steviol glycoside at its C-13 hydroxyl group (e.g., a UGT85C2 polypeptide); a gene encoding a polypeptide capable of beta 1,3 glycosylation of the C3′ of the 13-O-glucose, 19-O-glucose, or both 13-O-glucose and 19-O-glucose of a steviol glycoside (e.g., a UGT76G1 polypeptide); a gene encoding a polypeptide capable of glycosylating the steviol or the steviol glycoside at its C-19 carboxyl group (e.g., a UGT74G1 polypeptide); and/or a gene encoding a polypeptide capable of beta 1,2 glycosylation of the C2′ of the 13-O-glucose, 19-O-glucose, or both 13-O-glucose and 19-O-glucose of a steviol glycoside (e.g., a UGT91D2 or a EUGT11 polypeptide) can produce a steviol glycoside in vivo. The skilled worker will appreciate that one or more of these genes can be endogenous to the host provided that at least one (and in some embodiments, all) of these genes is a recombinant gene introduced into the recombinant host.

In some embodiments, steviol glycosides and/or steviol glycoside precursors are produced in vivo through expression of one or more enzymes involved in the steviol glycoside biosynthetic pathway in a recombinant host. For example, a recombinant host comprising a gene encoding a polypeptide capable of synthesizing GGPP from FPP and IPP; a gene encoding a polypeptide capable of synthesizing ent-copalyl diphosphate from GGPP; a gene encoding a polypeptide capable of synthesizing ent-kaurene from ent-copalyl diphosphate; a gene encoding a polypeptide capable of synthesizing ent-kaurenoic acid, ent-kaurenol, and/or ent-kaurenal from ent-kaurene; a gene encoding a polypeptide capable of reducing cytochrome P450 complex; a gene encoding a bifunctional polypeptide capable of synthesizing ent-copalyl diphosphate from GGPP and synthesizing ent-kaurene from ent-copalyl diphosphate; a gene encoding a polypeptide capable of glycosylating a steviol or a steviol glycoside at its C-13 hydroxyl group; a gene encoding a polypeptide capable of beta 1,3 glycosylation of the C3′ of the 13-O-glucose, 19-O-glucose, or both 13-O-glucose and 19-O-glucose of a steviol glycoside; a gene encoding a polypeptide capable of glycosylating the steviol or the steviol glycoside at its C-19 carboxyl group; and/or a gene encoding a polypeptide capable of beta 1,2 glycosylation of the C2′ of the 13-O-glucose, 19-O-glucose, or both 13-O-glucose and 19-O-glucose of a steviol glycoside can produce a steviol glycoside and/or steviol glycoside precursors in vivo. See, e.g., FIGS. 1 and 2. The skilled worker will appreciate that one or more of these genes can be endogenous to the host provided that at least one (and in some embodiments, all) of these genes is a recombinant gene introduced into the recombinant host.

In some embodiments, a steviol-producing recombinant microorganism comprises heterologous nucleic acids encoding a polypeptide capable of glycosylating a steviol or a steviol glycoside at its C-13 hydroxyl group; a polypeptide capable of beta 1,3 glycosylation of the C3′ of the 13-O-glucose, 19-O-glucose, or both 13-O-glucose and 19-O-glucose of a steviol glycoside; a polypeptide capable of glycosylating the steviol or the steviol glycoside at its C-19 carboxyl group; and a polypeptide capable of beta 1,2 glycosylation of the C2′ of the 13-O-glucose, 19-O-glucose, or both 13-O-glucose and 19-O-glucose of a steviol glycoside.

In some embodiments, a steviol-producing recombinant microorganism comprises heterologous nucleic acids encoding a polypeptide capable of glycosylating a steviol or a steviol glycoside at its C-13 hydroxyl group, a polypeptide capable of beta 1,3 glycosylation of the C3′ of the 13-O-glucose, 19-O-glucose, or both 13-O-glucose and 19-O-glucose of a steviol glycoside, and a polypeptide capable of beta 1,2 glycosylation of the C2′ of the 13-O-glucose, 19-O-glucose, or both 13-O-glucose and 19-O-glucose of a steviol glycoside polypeptides.

In some aspects, a polypeptide capable of glycosylating steviol or a steviol glycoside at its C-13 hydroxyl group, a polypeptide capable of beta 1,3 glycosylation of the C3′ of the 13-O-glucose, 19-O-glucose, or both 13-O-glucose and 19-O-glucose of a steviol glycoside, a polypeptide capable of glycosylating steviol or the steviol glycoside at its C-19 carboxyl group, and/or a polypeptide capable of beta 1,2 glycosylation of the C2′ of the 13-O-glucose, 19-O-glucose, or both 13-O-glucose and 19-O-glucose of a steviol glycoside, transfers a glucose molecule from uridine diphosphate glucose (UDP-glucose) to steviol and/or a steviol glycoside.

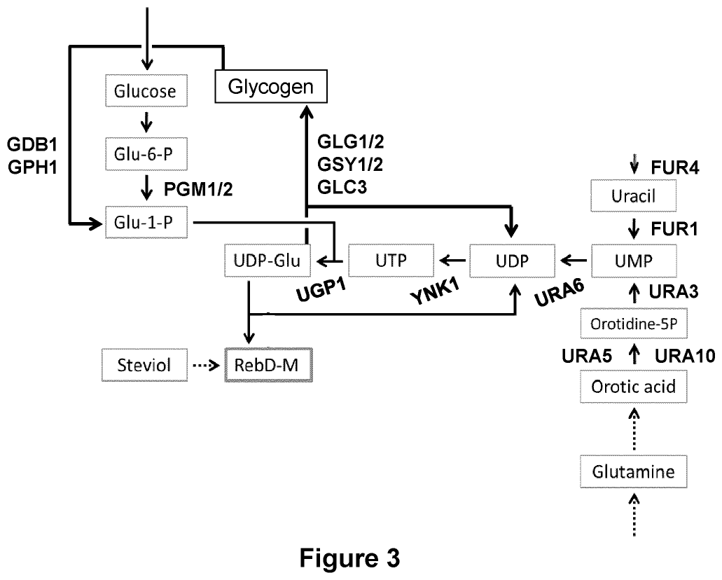

In some aspects, UDP-glucose is produced in vivo through expression of one or more enzymes involved in the UDP-glucose biosynthetic pathway in a recombinant host. For example, a recombinant host comprising a gene encoding a polypeptide capable of transporting uracil into the host cell (e.g., uracil permease (FUR4)); a gene encoding a polypeptide capable of synthesizing uridine monophosphate (UMP) from uracil (e.g., uracil phosphoribosyltransferase (FUR1)); a gene encoding a polypeptide capable of synthesizing orotidine monophosphate (OMP) from orotate or orotic acid (e.g., orotate phosphoribosyltransferase 1 (URA5) and orotate phosphoribosyltransferase 2 (URA10)); a gene encoding a polypeptide capable of synthesizing UMP from OMP (e.g., orotidine 5′-phosphate decarboxylase (URA3)); a gene encoding a polypeptide capable of synthesizing uridine diphosphate (UDP) from UMP (e.g., uridylate kinase (URA6)); a gene encoding a polypeptide capable of synthesizing uridine 5′-triphosphate (UTP) from UDP (i.e., a polypeptide capable of catalyzing the transfer of gamma phosphates from nucleoside triphosphates, e.g., nucleoside diphosphate kinase (YNK1)); a gene encoding a polypeptide capable of converting glucose-6-phosphate to glucose-1-phosphate (e.g., phosphoglucomutase-1 (PGM1) and phosphoglucomutase-2 (PGM2)); a gene encoding a polypeptide capable of debranching glycogen (e.g., glycogen debranching enzyme (GDB1)); a gene encoding a polypeptide capable of synthesizing glucose-1-phosphate from phosphate and glycogen (e.g., glycogen phosphorylase (GPH1)); and/or a gene encoding a polypeptide capable of synthesizing UDP-glucose from UTP and glucose-1-phosphate (e.g., UTP-glucose-1-phosphate uridylyltransferase (UGP1)) can produce UDP-glucose in vivo. See, e.g., FIG. 3. The skilled worker will appreciate that one or more of these genes may be endogenous to the host.

In some embodiments, a recombinant host comprises a gene encoding a polypeptide capable of synthesizing UTP from UDP. In some aspects, the gene encoding a polypeptide capable of synthesizing UTP from UDP is a recombinant gene. In some aspects, the recombinant gene comprises a nucleotide sequence native to the host. In other aspects, the recombinant gene comprises a heterologous nucleotide sequence. In some aspects, the recombinant gene is operably linked to a promoter. In some aspects, the recombinant gene is operably linked to a terminator, for example but not limited to, tCYC1 (SEQ ID NO:154) or tADH1 (SEQ ID NO:155). In some aspects, the promoter and terminator drive high expression of the recombinant gene. In some aspects, the recombinant gene is operably linked to a strong promoter, for example but not limited to, pTEF1 (SEQ ID NO:148), pPGK1 (SEQ ID NO:149), pTDH3 (SEQ ID NO:150), pTEF2 (SEQ ID NO:151), pTPI1 (SEQ ID NO:152), or pPDC1 (SEQ ID NO:153). In some aspects, the recombinant gene comprises a nucleotide sequence that originated from or is present in the same species as the recombinant host. In some aspects, expression of a recombinant gene encoding a polypeptide capable of synthesizing UTP from UDP results in a total expression level of genes encoding a polypeptide capable of synthesizing UTP from UDP that is higher than the expression level of endogenous genes encoding a polypeptide capable of synthesizing UTP from UDP, i.e., an overexpression of a polypeptide capable of synthesizing UTP from UDP.

In some aspects, the gene encoding the polypeptide capable of synthesizing UTP from UDP is a gene present in the same species as the recombinant host, i.e., an endogenous gene. In some embodiments, the wild-type promoter of an endogenous gene encoding the polypeptide capable of synthesizing UTP from UDP can be exchanged for a strong promoter. In some aspects, the strong promoter drives high expression of the endogenous gene (i.e., overexpression of the gene). In other embodiments, the wild-type enhancer of an endogenous gene encoding a polypeptide capable of synthesizing UTP from UDP can be exchanged for a strong enhancer. In some embodiments, the strong enhancer drives high expression of the endogenous gene (i.e., overexpression of the gene). In some embodiments, both the wild-type enhancer (i.e., operably linked to the promoter) and the wild-type promoter (i.e., operably linked to the endogenous gene) of the endogenous gene can be exchanged for a strong enhancer and strong promoter, respectively, resulting in overexpression of a polypeptide capable of synthesizing UTP from UDP (i.e., relative to the expression level of endogenous genes operably linked to wild-type enhancers and/or promoters). The endogenous gene operably linked to the strong enhancer and/or promoter may be located at the native loci, and/or may be located elsewhere in the genome.

For example, in some embodiments, a recombinant host comprising an endogenous gene encoding a polypeptide capable of synthesizing UTP from UDP, operably linked to a wild-type promoter, further comprises a recombinant gene encoding a polypeptide capable of synthesizing UTP from UDP, comprising a nucleotide sequence native to the host, operably linked to, e.g., a wild-type promoter, a promoter native to the host, or a heterologous promoter. In another example, in some embodiments, a recombinant host comprising an endogenous gene encoding a polypeptide capable of synthesizing UTP from UDP, operably linked to a wild-type promoter, further comprises a recombinant gene encoding a polypeptide capable of synthesizing UTP from UDP, comprising a heterologous nucleotide sequence, operably linked to, e.g., a wild-type promoter, a promoter native to the host, or a heterologous promoter. In yet another example, in some embodiments, a recombinant host comprises an endogenous gene encoding a polypeptide capable of synthesizing UTP from UDP, operably linked to, e.g., a strong promoter native to the host, or a heterologous promoter.

The person of ordinary skill in the art will appreciate that, e.g., expression of a recombinant gene encoding a polypeptide capable of synthesizing UTP from UDP; expression of a recombinant gene and an endogenous gene encoding a polypeptide capable of synthesizing UTP from UDP, and expression of an endogenous gene encoding a polypeptide capable of synthesizing UTP from UDP, wherein the wild-type promoter and/or enhancer of the endogenous gene are exchanged for a strong promoter and/or enhancer, each result in overexpression of a polypeptide capable of synthesizing UTP from UDP relative to a corresponding host not expressing a recombinant gene encoding a polypeptide capable of synthesizing UTP from UDP and/or a corresponding host expressing only a native gene encoding a polypeptide capable of synthesizing UTP from UDP, operably linked to the wild-type promoter and enhancer—i.e., as used herein, the term “expression” may include “overexpression.”

In some embodiments, a polypeptide capable of synthesizing UTP from UDP is overexpressed such that the total expression level of genes encoding the polypeptide capable of synthesizing UTP from UDP is at least 5% higher than the expression level of endogenous genes encoding a polypeptide capable of synthesizing UTP from UDP. In some embodiments, the total expression level of genes encoding a polypeptide capable of synthesizing UTP from UDP is at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% higher than the expression level of endogenous genes encoding a polypeptide capable of synthesizing UTP from UDP.

In some embodiments, a recombinant host comprises a gene encoding a polypeptide capable of converting glucose-6-phosphate to glucose-1-phosphate. In some aspects, the gene encoding a polypeptide capable of converting glucose-6-phosphate to glucose-1-phosphate is a recombinant gene. In some aspects, the recombinant gene comprises a nucleotide sequence native to the host. In other aspects, the recombinant gene comprises a heterologous nucleotide sequence. In some aspects, the recombinant gene is operably linked to a promoter. In some aspects, the recombinant gene is operably linked to a terminator, for example but not limited to, tCYC1 (SEQ ID NO:154) or tADH1 (SEQ ID NO:155). In some aspects, the promoter and terminator drive high expression of the recombinant gene. In some aspects, the recombinant gene is operably linked to a strong promoter, for example but not limited to, pTEF1 (SEQ ID NO:148), pPGK1 (SEQ ID NO:149), pTDH3 (SEQ ID NO:150), pTEF2 (SEQ ID NO:151), pTPI1 (SEQ ID NO:152), or pPDC1 (SEQ ID NO:153). In some aspects, the recombinant gene comprises a nucleotide sequence that originated from or is present in the same species as the recombinant host. In some aspects, expression of a recombinant gene encoding a polypeptide capable of converting glucose-6-phosphate to glucose-1-phosphate results in a total expression level of genes encoding a polypeptide capable of converting glucose-6-phosphate to glucose-1-phosphate that is higher than the expression level of endogenous genes encoding a polypeptide capable of converting glucose-6-phosphate to glucose-1-phosphate, i.e., an overexpression of a polypeptide capable of converting glucose-6-phosphate to glucose-1-phosphate.

In some aspects, the gene encoding the polypeptide capable of converting glucose-6-phosphate to glucose-1-phosphate is a gene present in the same species as the recombinant host, i.e., an endogenous gene. In some embodiments, the wild-type promoter of an endogenous gene encoding the polypeptide capable of converting glucose-6-phosphate to glucose-1-phosphate can be exchanged for a strong promoter. In some aspects, the strong promoter drives high expression of the endogenous gene (i.e., overexpression of the gene). In other embodiments, the wild-type enhancer of an endogenous gene encoding a polypeptide capable of converting glucose-6-phosphate to glucose-1-phosphate can be exchanged for a strong enhancer. In some embodiments, the strong enhancer drives high expression of the endogenous gene (i.e., overexpression of the gene). In some embodiments, both the wild-type enhancer (i.e., operably linked to the promoter) and the wild-type promoter (i.e., operably linked to the endogenous gene) of the endogenous gene can be exchanged for a strong enhancer and strong promoter, respectively, resulting in overexpression of a polypeptide capable of converting glucose-6-phosphate to glucose-1-phosphate (i.e., relative to the expression level of endogenous genes operably linked to wild-type enhancers and/or promoters). The endogenous gene operably linked to the strong enhancer and/or promoter may be located at the native loci, and/or may be located elsewhere in the genome.

For example, in some embodiments, a recombinant host comprising an endogenous gene encoding a polypeptide capable of converting glucose-6-phosphate to glucose-1-phosphate, operably linked to a wild-type promoter, further comprises a recombinant gene encoding a polypeptide capable of converting glucose-6-phosphate to glucose-1-phosphate, comprising a nucleotide sequence native to the host, operably linked to, e.g., a wild-type promoter, a promoter native to the host, or a heterologous promoter. In another example, in some embodiments, a recombinant host comprising an endogenous gene encoding a polypeptide capable of converting glucose-6-phosphate to glucose-1-phosphate, operably linked to a wild-type promoter, further comprises a recombinant gene encoding a polypeptide capable of converting glucose-6-phosphate to glucose-1-phosphate, comprising a heterologous nucleotide sequence, operably linked to, e.g., a wild-type promoter, a promoter native to the host, or a heterologous promoter. In yet another example, in some embodiments, a recombinant host comprises an endogenous gene encoding a polypeptide capable of converting glucose-6-phosphate to glucose-1-phosphate, operably linked to, e.g., a strong promoter native to the host, or a heterologous promoter.

In some embodiments, a polypeptide capable of converting glucose-6-phosphate to glucose-1-phosphate is overexpressed such that the total expression level of genes encoding the polypeptide capable of converting glucose-6-phosphate to glucose-1-phosphate is at least 5% higher than the expression level of endogenous genes encoding a polypeptide capable of converting glucose-6-phosphate to glucose-1-phosphate. In some embodiments, the total expression level of genes encoding a polypeptide capable of converting glucose-6-phosphate to glucose-1-phosphate is at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% higher than the expression level of endogenous genes encoding a polypeptide capable of converting glucose-6-phosphate to glucose-1-phosphate.

In some embodiments, a recombinant host comprises a gene encoding a polypeptide capable of debranching glycogen. In some aspects, debranching glycogen comprises glycogen breakdown and/or glucose mobilization. In some aspects, debranching glycogen comprises breakdown of glycogen into glucose-1-phosphate. In some aspects, the polypeptide capable of debranching glycogen comprises a polypeptide capable of intramolecularly transferring α-1,4-linked glucose and/or α-1,4-linked glucan of glycogen to a new position (i.e., 4-α-glucanotransferase activity), and/or capable of hydrolyzing an α-1,6 linkage of glycogen (i.e., α-1,6-amyloglucosidase activity). In some aspects, the polypeptide capable of debranching glycogen comprises a bifunctional polypeptide capable of 4-α-glucanotransferase activity and capable of α-1,6-amyloglucosidase activity. In some aspects, the recombinant host can comprise a first polypeptide capable of 4-α-glucanotransferase activity and a second peptide capable of α-1,6-amyloglucosidase activity. In some aspects, the gene encoding a polypeptide capable of debranching glycogen is a recombinant gene. In some aspects, the recombinant gene comprises a nucleotide sequence native to the host. In other aspects, the recombinant gene comprises a heterologous nucleotide sequence. In some aspects, the recombinant gene is operably linked to a promoter. In some aspects, the recombinant gene is operably linked to a terminator, for example but not limited to, tCYC1 (SEQ ID NO:154) or tADH1 (SEQ ID NO:155). In some aspects, the promoter and terminator drive high expression of the recombinant gene. In some aspects, the recombinant gene is operably linked to a strong promoter, for example but not limited to, pTEF1 (SEQ ID NO:148), pPGK1 (SEQ ID NO:149), pTDH3 (SEQ ID NO:150), pTEF2 (SEQ ID NO:151), pTPI1 (SEQ ID NO:152), or pPDC1 (SEQ ID NO:153). In some aspects, the recombinant gene comprises a nucleotide sequence that originated from or is present in the same species as the recombinant host. In some aspects, expression of a recombinant gene encoding a polypeptide capable of debranching glycogen results in a total expression level of genes encoding a polypeptide capable of debranching glycogen that is higher than the expression level of endogenous genes encoding a polypeptide capable of debranching glycogen, i.e., an overexpression of a polypeptide capable of debranching glycogen.

In some aspects, the gene encoding the polypeptide capable of debranching glycogen is a gene present in the same species as the recombinant host, i.e., an endogenous gene. In some embodiments, the wild-type promoter of an endogenous gene encoding the polypeptide capable of debranching glycogen can be exchanged for a strong promoter. In some aspects, the strong promoter drives high expression of the endogenous gene (i.e., overexpression of the gene). In other embodiments, the wild-type enhancer of an endogenous gene encoding a polypeptide capable of debranching glycogen can be exchanged for a strong enhancer. In some embodiments, the strong enhancer drives high expression of the endogenous gene (i.e., overexpression of the gene). In some embodiments, both the wild-type enhancer (i.e., operably linked to the promoter) and the wild-type promoter (i.e., operably linked to the endogenous gene) of the endogenous gene can be exchanged for a strong enhancer and strong promoter, respectively, resulting in overexpression of a polypeptide capable of debranching glycogen (i.e., relative to the expression level of endogenous genes operably linked to wild-type enhancers and/or promoters). The endogenous gene operably linked to the strong enhancer and/or promoter may be located at the native loci, and/or may be located elsewhere in the genome.

For example, in some embodiments, a recombinant host comprising an endogenous gene encoding a polypeptide capable of debranching glycogen, operably linked to a wild-type promoter, further comprises a recombinant gene encoding a polypeptide capable of debranching glycogen, comprising a nucleotide sequence native to the host, operably linked to, e.g., a wild-type promoter, a promoter native to the host, or a heterologous promoter. In another example, in some embodiments, a recombinant host comprising an endogenous gene encoding a polypeptide capable of debranching glycogen, operably linked to a wild-type promoter, further comprises a recombinant gene encoding a polypeptide capable of debranching glycogen, comprising a heterologous nucleotide sequence, operably linked to, e.g., a wild-type promoter, a promoter native to the host, or a heterologous promoter. In yet another example, in some embodiments, a recombinant host comprises an endogenous gene encoding a polypeptide capable of debranching glycogen, operably linked to, e.g., a strong promoter native to the host, or a heterologous promoter.

In some embodiments, a polypeptide capable of debranching glycogen is overexpressed such that the total expression level of genes encoding the polypeptide capable of debranching glycogen is at least 5% higher than the expression level of endogenous genes encoding a polypeptide capable of debranching glycogen. In some embodiments, the total expression level of genes encoding a polypeptide capable of debranching glycogen is at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% higher than the expression level of endogenous genes encoding a polypeptide capable of debranching glycogen.

In some embodiments, a recombinant host comprises a gene encoding a polypeptide capable of synthesizing glucose-1-phosphate from phosphate and glycogen. In some aspects, the gene encoding a polypeptide capable of synthesizing glucose-1-phosphate from phosphate and glycogen comprises a polypeptide capable of synthesizing glucose-1-phosphate from phosphate and an α-1,4-linked glucose of glycogen. In some aspects, the gene encoding a polypeptide capable of synthesizing glucose-1-phosphate from phosphate and glycogen is a recombinant gene. In some aspects, the recombinant gene comprises a nucleotide sequence native to the host. In other aspects, the recombinant gene comprises a heterologous nucleotide sequence. In some aspects, the recombinant gene is operably linked to a promoter. In some aspects, the recombinant gene is operably linked to a terminator, for example but not limited to, tCYC1 (SEQ ID NO:154) or tADH1 (SEQ ID NO:155). In some aspects, the promoter and terminator drive high expression of the recombinant gene. In some aspects, the recombinant gene is operably linked to a strong promoter, for example but not limited to, pTEF1 (SEQ ID NO:148), pPGK1 (SEQ ID NO:149), pTDH3 (SEQ ID NO:150), pTEF2 (SEQ ID NO:151), pTPI1 (SEQ ID NO:152), or pPDC1 (SEQ ID NO:153). In some aspects, the recombinant gene comprises a nucleotide sequence that originated from or is present in the same species as the recombinant host. In some aspects, expression of a recombinant gene encoding a polypeptide capable of synthesizing glucose-1-phosphate from phosphate and glycogen results in a total expression level of genes encoding a polypeptide capable of synthesizing glucose-1-phosphate from phosphate and glycogen that is higher than the expression level of endogenous genes encoding a polypeptide capable of synthesizing glucose-1-phosphate from phosphate and glycogen, i.e., an overexpression of a polypeptide capable of synthesizing glucose-1-phosphate from phosphate and glycogen.

In some aspects, the gene encoding the polypeptide capable of synthesizing glucose-1-phosphate from phosphate and glycogen is a gene present in the same species as the recombinant host, i.e., an endogenous gene. In some embodiments, the wild-type promoter of an endogenous gene encoding the polypeptide capable of synthesizing glucose-1-phosphate from phosphate and glycogen can be exchanged for a strong promoter. In some aspects, the strong promoter drives high expression of the endogenous gene (i.e., overexpression of the gene). In other embodiments, the wild-type enhancer of an endogenous gene encoding a polypeptide capable of synthesizing glucose-1-phosphate from phosphate and glycogen can be exchanged for a strong enhancer. In some embodiments, the strong enhancer drives expression of the endogenous gene (i.e., overexpression of the gene). In some embodiments, both the wild-type enhancer (i.e., operably linked to the promoter) and the wild-type promoter (i.e., operably linked to the endogenous gene) of the endogenous gene can be exchanged for a strong enhancer and strong promoter, respectively, resulting in overexpression of a polypeptide capable of synthesizing glucose-1-phosphate from phosphate and glycogen (i.e., relative to the expression level of endogenous genes operably linked to wild-type enhancers and/or promoters). The endogenous gene operably linked to the strong enhancer and/or promoter may be located at the native loci, and/or may be located elsewhere in the genome.

For example, in some embodiments, a recombinant host comprising an endogenous gene encoding a polypeptide capable of synthesizing glucose-1-phosphate from phosphate and glycogen, operably linked to a wild-type promoter, further comprises a recombinant gene encoding a polypeptide capable of synthesizing glucose-1-phosphate from phosphate and glycogen, comprising a nucleotide sequence native to the host, operably linked to, e.g., a wild-type promoter, a promoter native to the host, or a heterologous promoter. In another example, in some embodiments, a recombinant host comprising an endogenous gene encoding a polypeptide capable of synthesizing glucose-1-phosphate from phosphate and glycogen, operably linked to a wild-type promoter, further comprises a recombinant gene encoding a polypeptide capable of synthesizing glucose-1-phosphate from phosphate and glycogen, comprising a heterologous nucleotide sequence, operably linked to, e.g., a wild-type promoter, a promoter native to the host, or a heterologous promoter. In yet another example, in some embodiments, a recombinant host comprises an endogenous gene encoding a polypeptide capable of synthesizing glucose-1-phosphate from phosphate and glycogen, operably linked to, e.g., a strong promoter native to the host, or a heterologous promoter.

In some embodiments, a polypeptide capable of synthesizing glucose-1-phosphate from phosphate and glycogen is overexpressed such that the total expression level of genes encoding the polypeptide capable of synthesizing glucose-1-phosphate from phosphate and glycogen is at least 5% higher than the expression level of endogenous genes encoding a polypeptide capable of synthesizing glucose-1-phosphate from phosphate and glycogen. In some embodiments, the total expression level of genes encoding a polypeptide capable of synthesizing glucose-1-phosphate from phosphate and glycogen is at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% higher than the expression level of endogenous genes encoding a polypeptide capable of synthesizing glucose-1-phosphate from phosphate and glycogen.

In some embodiments, a recombinant host comprises a gene encoding a polypeptide capable of synthesizing UDP-glucose from UTP and glucose-1-phosphate. In some aspects, the gene encoding a polypeptide capable of synthesizing UDP-glucose from UTP and glucose-1-phosphate is a recombinant gene. In some aspects, the recombinant gene comprises a nucleotide sequence native to the host. In other aspects, the recombinant gene comprises a heterologous nucleotide sequence. In some aspects, the recombinant gene is operably linked to a promoter. In some aspects, the recombinant gene is operably linked to a terminator, for example but not limited to, tCYC1 (SEQ ID NO:154) or tADH1 (SEQ ID NO:155). In some aspects, the promoter and terminator drive high expression of the recombinant gene. In some aspects, the recombinant gene is operably linked to a strong promoter, for example but not limited to, pTEF1 (SEQ ID NO:148), pPGK1 (SEQ ID NO:149), pTDH3 (SEQ ID NO:150), pTEF2 (SEQ ID NO:151), pTPI1 (SEQ ID NO:152), or pPDC1 (SEQ ID NO:153). In some aspects, the recombinant gene comprises a nucleotide sequence that originated from or is present in the same species as the recombinant host. In some aspects, expression of a recombinant gene encoding a polypeptide capable of synthesizing UDP-glucose from UTP and glucose-1-phosphate results in a total expression level of genes encoding a polypeptide capable of synthesizing UDP-glucose from UTP and glucose-1-phosphate that is higher than the expression level of endogenous genes encoding a polypeptide capable of synthesizing UDP-glucose from UTP and glucose-1-phosphate, i.e., an overexpression of a polypeptide capable of synthesizing UDP-glucose from UTP and glucose-1-phosphate.

In some aspects, the gene encoding the polypeptide capable of synthesizing UDP-glucose from UTP and glucose-1-phosphate is a gene present in the same species as the recombinant host, i.e., an endogenous gene. In some embodiments, the wild-type promoter of an endogenous gene encoding the polypeptide capable of synthesizing UDP-glucose from UTP and glucose-1-phosphate can be exchanged for a strong promoter. In some aspects, the strong promoter drives high expression of the endogenous gene (i.e., overexpression of the gene). In other embodiments, the wild-type enhancer of an endogenous gene encoding a polypeptide capable of synthesizing UDP-glucose from UTP and glucose-1-phosphate can be exchanged for a strong enhancer. In some embodiments, the strong enhancer drives high expression of the endogenous gene (i.e., overexpression of the gene). In some embodiments, both the wild-type enhancer (i.e., operably linked to the promoter) and the wild-type promoter (i.e., operably linked to the endogenous gene) of the endogenous gene can be exchanged for a strong enhancer and strong promoter, respectively, resulting in overexpression of a polypeptide capable of synthesizing UDP-glucose from UTP and glucose-1-phosphate (i.e., relative to the expression level of endogenous genes operably linked to wild-type enhancers and/or promoters). The endogenous gene operably linked to the strong enhancer and/or promoter may be located at the native loci, and/or may be located elsewhere in the genome.

For example, in some embodiments, a recombinant host comprising an endogenous gene encoding a polypeptide capable of synthesizing UDP-glucose from UTP and glucose-1-phosphate, operably linked to a wild-type promoter, further comprises a recombinant gene encoding a polypeptide capable of synthesizing UDP-glucose from UTP and glucose-1-phosphate, comprising a nucleotide sequence native to the host, operably linked to, e.g., a wild-type promoter, a promoter native to the host, or a heterologous promoter. In another example, in some embodiments, a recombinant host comprising an endogenous gene encoding a polypeptide capable of synthesizing UDP-glucose from UTP and glucose-1-phosphate, operably linked to a wild-type promoter, further comprises a recombinant gene encoding a polypeptide capable of synthesizing UDP-glucose from UTP and glucose-1-phosphate, comprising a heterologous nucleotide sequence, operably linked to, e.g., a wild-type promoter, a promoter native to the host, or a heterologous promoter. In yet another example, in some embodiments, a recombinant host comprises an endogenous gene encoding a polypeptide capable of synthesizing UDP-glucose from UTP and glucose-1-phosphate, operably linked to, e.g., a strong promoter native to the host, or a heterologous promoter.

In some embodiments, a recombinant host comprising a polypeptide capable of synthesizing UDP-glucose from UTP and glucose-1-phosphate is overexpressed such that the total expression level of genes encoding the polypeptide capable of synthesizing UDP-glucose from UTP and glucose-1-phosphate is at least 5% higher than the expression level of endogenous genes encoding a polypeptide capable of synthesizing UDP-glucose from UTP and glucose-1-phosphate. In some embodiments, the total expression level of genes encoding a polypeptide capable of synthesizing UDP-glucose from UTP and glucose-1-phosphate is at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% higher than the expression level of endogenous genes encoding a polypeptide capable of synthesizing UDP-glucose from UTP and glucose-1-phosphate.

In some aspects, a recombinant host comprising one or more genes encoding one or more polypeptides capable of synthesizing UTP from UDP, one or more genes encoding one or more polypeptides capable of converting glucose-6-phosphate to glucose-1-phosphate, one or more genes encoding one or more polypeptide capable of debranching glycogen, one or more genes encoding one or more polypeptides capable of synthesizing glucose-1-phosphate from phosphate and glycogen, and/or one or more genes encoding one or more polypeptides capable of synthesizing UDP-glucose from UTP and glucose-1-phosphate may further comprise a recombinant gene encoding a polypeptide capable of transporting uracil into the host cell; a recombinant gene encoding a polypeptide capable of synthesizing uridine monophosphate (UMP) from uracil; a recombinant gene encoding a polypeptide capable of synthesizing orotidine monophosphate (OMP) from orotate or orotic acid; a recombinant gene encoding a polypeptide capable of synthesizing UMP from OMP; and/or a recombinant gene encoding a polypeptide capable of synthesizing uridine diphosphate (UDP) from UMP. In some embodiments, a recombinant host comprising one or more genes encoding one or more polypeptides capable of synthesizing UTP from UDP, one or more genes encoding one or more polypeptides capable of converting glucose-6-phosphate to glucose-1-phosphate, one or more genes encoding one or more polypeptides capable of debranching glycogen, one or more genes encoding one or more polypeptides capable of synthesizing glucose-1-phosphate from phosphate and glycogen, and/or one or more genes encoding one or more polypeptides capable of synthesizing UDP-glucose from UTP and glucose-1-phosphate may overexpress a gene encoding a polypeptide capable of transporting uracil into the host cell; a gene encoding a polypeptide capable of synthesizing uridine monophosphate (UMP) from uracil; a gene encoding a polypeptide capable of synthesizing orotidine monophosphate (OMP) from orotate or orotic acid; a gene encoding a polypeptide capable of synthesizing UMP from OMP; and/or a gene encoding a polypeptide capable of synthesizing uridine diphosphate (UDP) from UMP.

In some aspects, the polypeptide capable of synthesizing UTP from UDP comprises a polypeptide having the amino acid sequence set forth in SEQ ID NO:123 (which can be encoded by the nucleotide sequence set forth in SEQ ID NO:122).

In some aspects, the polypeptide capable of converting glucose-6-phosphate to glucose-1-phosphate comprises a polypeptide having the amino acid sequence set forth in SEQ ID NO:2 (which can be encoded by the nucleotide sequence set forth in SEQ ID NO:1), SEQ ID NO:119 (encoded by the nucleotide sequence set forth in SEQ ID NO:118), SEQ ID NO:141 (encoded by the nucleotide sequence set forth in SEQ ID NO:140), SEQ ID NO:143 (encoded by the nucleotide sequence set forth in SEQ ID NO:142), SEQ ID NO:145 (encoded by the nucleotide sequence set forth in SEQ ID NO:144), or SEQ ID NO:147 (encoded by the nucleotide sequence set forth in SEQ ID NO:146).

In some aspects, the polypeptide capable of debranching glycogen comprises a polypeptide having the amino acid sequence set forth in SEQ ID NO:157 (which can be encoded by the nucleotide sequence set forth in SEQ ID NO:156).

In some aspects, the polypeptide capable of synthesizing glucose-1-phosphate from phosphate and glycogen comprises a polypeptide having the amino acid sequence set forth in SEQ ID NO:159 (which can be encoded by the nucleotide sequence set forth in SEQ ID NO:158).

In some aspects, the polypeptide capable of synthesizing UDP-glucose from UTP and glucose-1-phosphate comprises a polypeptide having the amino acid sequence set forth in SEQ ID NO:121 (which can be encoded by the nucleotide sequence set forth in SEQ ID NO:120), SEQ ID NO:125 (encoded by the nucleotide sequence set forth in SEQ ID NO:124), SEQ ID NO:127 (encoded by the nucleotide sequence set forth in SEQ ID NO:126), SEQ ID NO:129 (encoded by the nucleotide sequence set forth in SEQ ID NO:128), SEQ ID NO:131 (encoded by the nucleotide sequence set forth in SEQ ID NO:130), SEQ ID NO:133 (encoded by the nucleotide sequence set forth in SEQ ID NO:132), SEQ ID NO:135 (encoded by the nucleotide sequence set forth in SEQ ID NO:134), SEQ ID NO:137 (encoded by the nucleotide sequence set forth in SEQ ID NO:136), or SEQ ID NO:139 (encoded by the nucleotide sequence set forth in SEQ ID NO:138).

In some embodiments, a recombinant host comprises a recombinant gene encoding a polypeptide capable of synthesizing UTP from UDP and a recombinant gene encoding a polypeptide capable of converting glucose-6-phosphate to glucose-1-phosphate. In some embodiments, a recombinant host comprises a recombinant gene encoding a polypeptide capable of synthesizing UTP from UDP and a recombinant gene encoding a polypeptide capable of synthesizing UDP-glucose from UTP and glucose-1-phosphate. In some embodiments, a recombinant host comprises a recombinant gene encoding a polypeptide capable of converting glucose-6-phosphate to glucose-1-phosphate and a recombinant gene encoding a polypeptide capable of synthesizing UDP-glucose from UTP and glucose-1-phosphate. In some embodiments, a recombinant host comprises a recombinant gene encoding a polypeptide capable of synthesizing UTP from UDP, a recombinant gene encoding a polypeptide capable of converting glucose-6-phosphate to glucose-1-phosphate, and a recombinant gene encoding a polypeptide capable of synthesizing UDP-glucose from UTP and glucose-1-phosphate.

In some embodiments, a recombinant host comprises a recombinant gene encoding a polypeptide capable of debranching glycogen and a recombinant gene encoding a polypeptide capable of synthesizing glucose-1-phosphate from phosphate and glycogen. In some embodiments, a recombinant host comprises a recombinant gene encoding a polypeptide capable of debranching glycogen, a recombinant gene encoding a polypeptide capable of synthesizing glucose-1-phosphate from phosphate and glycogen, a polypeptide capable of synthesizing UTP from UDP, and a recombinant gene encoding a polypeptide capable of converting glucose-6-phosphate to glucose-1-phosphate. In some embodiments, a recombinant host comprises a recombinant gene encoding a polypeptide capable of debranching glycogen, a recombinant gene encoding a polypeptide capable of synthesizing glucose-1-phosphate from phosphate and glycogen, a recombinant gene encoding a polypeptide capable of synthesizing UTP from UDP, and a recombinant gene encoding a polypeptide capable of synthesizing UDP-glucose from UTP and glucose-1-phosphate. In some embodiments, a recombinant host comprises a recombinant gene encoding a polypeptide capable of debranching glycogen, a recombinant gene encoding a polypeptide capable of synthesizing glucose-1-phosphate from phosphate and glycogen, a recombinant gene encoding a polypeptide capable of converting glucose-6-phosphate to glucose-1-phosphate, and a recombinant gene encoding a polypeptide capable of synthesizing UDP-glucose from UTP and glucose-1-phosphate. In some embodiments, a recombinant host comprises a recombinant gene encoding a polypeptide capable of debranching glycogen, a recombinant gene encoding a polypeptide capable of synthesizing glucose-1-phosphate from phosphate and glycogen, a recombinant gene encoding a polypeptide capable of synthesizing UTP from UDP, a recombinant gene encoding a polypeptide capable of converting glucose-6-phosphate to glucose-1-phosphate, and a recombinant gene encoding a polypeptide capable of synthesizing UDP-glucose from UTP and glucose-1-phosphate.

In some embodiments, a recombinant host comprises two or more recombinant genes encoding a polypeptide involved in the UDP-glucose biosynthetic pathway, e.g., a gene encoding a polypeptide capable of converting glucose-6-phosphate having a first amino acid sequence and a gene encoding a polypeptide capable of converting glucose-6-phosphate having a second amino acid sequence distinct from the first amino acid sequence. For example, in some embodiments, a recombinant host comprises a gene encoding a polypeptide having the amino acid sequence of PGM1 (e.g., a polypeptide having the amino acid sequence set forth in SEQ ID NO:2) and a gene encoding a polypeptide having the amino acid sequence of PGM2 (e.g., a polypeptide having the amino acid sequence set forth in SEQ ID NO:119, SEQ ID NO:141, SEQ ID NO:143, SEQ ID NO:145, or SEQ ID NO:147). In certain such embodiments, the two or more genes encoding a polypeptide involved in the UDP-glucose biosynthetic pathway comprise nucleotide sequences native to the recombinant host cell (e.g., a recombinant S. cerevisiae host cell comprising a gene encoding a polypeptide having the amino acid sequence set forth in SEQ ID NO:2 and a gene encoding a polypeptide having the amino acid sequence set forth in SEQ ID NO:119). In other such embodiments, one of the two or more genes encoding a polypeptide involved in the UDP-glucose biosynthetic pathway comprises a nucleotide sequence native to the recombinant host cell, while one or more of the two or more genes encoding a polypeptide involved in the UDP-glucose biosynthetic pathway comprises a heterologous nucleotide sequence. For example, in some embodiments, a recombinant S. cerevisiae host cell expressing a recombinant gene encoding a polypeptide capable of synthesizing UDP-glucose from UTP and glucose-1-phosphate having the amino acid sequence set forth in SEQ ID NO:121 (i.e., a recombinant host overexpressing the polypeptide) further expresses a recombinant gene encoding a polypeptide capable of synthesizing UDP-glucose from UTP and glucose-1-phosphate having the amino acid sequence set forth in, e.g., SEQ ID NO:125, SEQ ID NO:127, SEQ ID NO:129, SEQ ID NO:131, SEQ ID NO:133, SEQ ID NO:135, SEQ ID NO:137, or SEQ ID NO:139. In another example, in some embodiments, a recombinant S. cerevisiae host cell expressing a recombinant gene encoding a polypeptide capable of converting glucose-6-phosphate to glucose-1-phosphate having the amino acid sequence set forth in SEQ ID NO:119 (i.e., a recombinant host overexpressing the polypeptide) further expresses a recombinant gene encoding a polypeptide capable of converting glucose-6-phosphate to glucose-1-phosphate having the amino acid sequence set forth in, e.g., SEQ ID NO:141, SEQ ID NO:143, SEQ ID NO:145, or SEQ ID NO:147. Accordingly, as used herein, the term “a recombinant gene” may include “one or more recombinant genes.”

In some embodiments, a recombinant host comprises two or more copies of a recombinant gene encoding a polypeptide involved in the UDP-glucose biosynthetic pathway or the steviol glycoside biosynthetic pathway. In some embodiments, a recombinant host is preferably transformed with, e.g., two copies, three copies, four copies, or five copies of a recombinant gene encoding a polypeptide involved in the UDP-glucose biosynthetic pathway or the steviol glycoside biosynthetic pathway. For example, in some embodiments, a recombinant host is transformed with two copies of a recombinant gene encoding a polypeptide capable of synthesizing UTP from UDP (e.g., a polypeptide having the amino acid sequence set forth in SEQ ID NO:123), two copies of a recombinant gene encoding a polypeptide capable of debranching glycogen (e.g., a polypeptide having the amino acid sequence set forth in SEQ ID NO:157), or two copies of a recombinant gene encoding a polypeptide capable of synthesizing glucose-1-phosphate from phosphate and glycogen (e.g., a polypeptide having the amino acid sequence set forth in SEQ ID NO:159). The person of ordinary skill in the art will appreciate that, in some embodiments, recombinant genes may be replicated in a host cell independently of cell replication; accordingly, a recombinant host cell may comprise, e.g., more copies of a recombinant gene than the number of copies the cell was transformed with. Accordingly, as used herein, the term “a recombinant gene” may include “one or more copies of a recombinant gene.”

In some aspects, expression of a polypeptide capable of synthesizing UTP from UDP, a polypeptide capable of converting glucose-6-phosphate to glucose-1-phosphate, a polypeptide capable of debranching glycogen, a polypeptide capable of synthesizing glucose-1-phosphate from phosphate and glycogen, and/or a polypeptide capable of synthesizing UDP-glucose from UTP and glucose-1-phosphate in a recombinant host cell increases the amount of UDP-glucose produced by the cell. In some aspects, expression of a polypeptide capable of synthesizing UTP from UDP, a polypeptide capable of converting glucose-6-phosphate to glucose-1-phosphate, a polypeptide capable of debranching glycogen, a polypeptide capable of synthesizing glucose-1-phosphate from phosphate and glycogen, and/or a polypeptide capable of synthesizing UDP-glucose from UTP and glucose-1-phosphate in a recombinant host cell maintains, or even increases, the pool of UDP-glucose available for, e.g., glycosylation of a steviol or a steviol glycoside. In some aspects, expression of a polypeptide capable of synthesizing UTP from UDP, a polypeptide capable of converting glucose-6-phosphate to glucose-1-phosphate, a polypeptide capable of debranching glycogen, a polypeptide capable of synthesizing glucose-1-phosphate from phosphate and glycogen, and/or a polypeptide capable of synthesizing UDP-glucose from UTP and glucose-1-phosphate in a recombinant host cell increases the speed with which UDP-glucose is regenerated, thus maintaining, or even increasing, the UDP-glucose pool, which can be used to synthesize one or more steviol glycosides.

In some embodiments, expression of a recombinant gene encoding a polypeptide capable of debranching glycogen (e.g., a polypeptide having the amino acid sequence set forth in SEQ ID NO:157) and a recombinant gene encoding a polypeptide capable of synthesizing glucose-1-phosphate from phosphate and glycogen (e.g., a polypeptide having the amino acid sequence set forth in SEQ ID NO:159) in a recombinant host cell increases the amount of UDP-glucose produced by the cell by at least 10%, e.g., at least 25%, or at least 50%, or at least 75%, or at least 100%, or at least 125%, or at least 150%, or at least 175%, or at least 200%, or at least 225%, or at least 250%, or at least 275%, or at least 300%, calculated as an increase in intracellular UDP-glucose concentration relative to a corresponding host lacking the recombinant genes.

In some embodiments, expression of a recombinant gene encoding a polypeptide capable of synthesizing UTP from UDP (e.g., a polypeptide having the amino acid sequence set forth in SEQ ID NO:123), a recombinant gene encoding a polypeptide capable of converting glucose-6-phosphate to glucose-1-phosphate (e.g. a polypeptide having the amino acid sequence set forth in SEQ ID NO:2, SEQ ID NO:119, SEQ ID NO:141, SEQ ID NO:143, SEQ ID NO:145, or SEQ ID NO:147), a recombinant gene encoding a polypeptide capable of debranching glycogen (e.g., a polypeptide having the amino acid sequence set forth in SEQ ID NO:157), a recombinant gene encoding a polypeptide capable of synthesizing glucose-1-phosphate from phosphate and glycogen (e.g., a polypeptide having the amino acid sequence set forth in SEQ ID NO:159), and a recombinant gene encoding a polypeptide capable of synthesizing UDP-glucose from UTP and glucose-1-phosphate (e.g., a polypeptide having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, or SEQ ID NO:139) in a recombinant host cell increases the amount of UDP-glucose produced by the cell by at least 10%, e.g., at least 25%, or at least 50%, or at least 75%, or at least 100%, or at least 125%, or at least 150%, or at least 175%, or at least 200%, or at least 225%, or at least 250%, or at least 275%, or at least 300%, calculated as an increase in intracellular UDP-glucose concentration relative to a corresponding host lacking the recombinant genes.

In certain such embodiments, one or more of the recombinant gene encoding a polypeptide capable of synthesizing UTP from UDP, the recombinant gene encoding a polypeptide capable of converting glucose-6-phosphate to glucose-1-phosphate, the recombinant gene encoding a polypeptide capable of debranching glycogen, the recombinant gene encoding a polypeptide capable of synthesizing glucose-1-phosphate from phosphate and glycogen, and the recombinant gene encoding a polypeptide capable of synthesizing UDP-glucose from UTP and glucose-1-phosphate comprise a nucleotide sequence native to the host cell. For example, in some embodiments, expression of a recombinant gene encoding a polypeptide capable of debranching glycogen having the amino acid sequence set forth in SEQ ID NO:157 and a recombinant gene encoding a polypeptide capable of synthesizing glucose-1-phosphate from phosphate and glycogen having the amino acid sequence set forth in SEQ ID NO:159 in a steviol glycoside-producing S. cerevisiae host cell (i.e., providing a recombinant host overexpressing the polypeptides) increases the amount of UDP-glucose produced by the cell by at least 10%, e.g., at least 25%, or at least 50%, or at least 75%, or at least 100%, or at least 125%, or at least 150%, or at least 175%, or at least 200%, or at least 225%, or at least 250%, or at least 275%, or at least 300%, calculated as an increase in intracellular UDP-glucose concentration relative to a corresponding host lacking the recombinant genes.

In another example, in some embodiments, expression of a recombinant gene encoding a polypeptide capable of synthesizing UTP from UDP having the amino acid sequence set forth in SEQ ID NO:123, a recombinant gene encoding a polypeptide capable of converting glucose-6-phosphate to glucose-1-phosphate having the amino acid sequence set forth in SEQ ID NO:2 and/or SEQ ID NO:119, a recombinant gene encoding a polypeptide capable of debranching glycogen having the amino acid sequence set forth in SEQ ID NO:157, a recombinant gene encoding a polypeptide capable of synthesizing glucose-1-phosphate from phosphate and glycogen having the amino acid sequence set forth in SEQ ID NO:159, and a recombinant gene encoding a polypeptide capable of synthesizing UDP-glucose from UTP and glucose-1-phosphate having the amino acid sequence set forth in SEQ ID NO:121 in a steviol glycoside-producing S. cerevisiae host cell (i.e., providing a recombinant host overexpressing the polypeptides) increases the amount of UDP-glucose produced by the cell by at least 10%, e.g., at least 25%, or at least 50%, or at least 75%, or at least 100%, or at least 125%, or at least 150%, or at least 175%, or at least 200%, or at least 225%, or at least 250%, or at least 275%, or at least 300%, calculated as an increase in intracellular UDP-glucose concentration relative to a corresponding host lacking the recombinant genes.

In some aspects, expression of a polypeptide capable of synthesizing UTP from UDP, a polypeptide capable of converting glucose-6-phosphate to glucose-1-phosphate, a polypeptide capable of debranching glycogen, a polypeptide capable of synthesizing glucose-1-phosphate from phosphate and glycogen, and/or a polypeptide capable of synthesizing UDP-glucose from UTP and glucose-1-phosphate in a steviol-glycoside producing recombinant host cell further expressing a gene encoding a polypeptide capable of glycosylating a steviol or a steviol glycoside at its C-13 hydroxyl group; a gene encoding a polypeptide capable of beta 1,3 glycosylation of the C3′ of the 13-O-glucose, 19-O-glucose, or both 13-O-glucose and 19-O-glucose of a steviol glycoside; a gene encoding a polypeptide capable of glycosylating the steviol or the steviol glycoside at its C-19 carboxyl group; and/or a gene encoding a polypeptide capable of beta 1,2 glycosylation of the C2′ of the 13-O-glucose, 19-O-glucose, or both 13-O-glucose and 19-O-glucose of a steviol glycoside, increases the amount of one or more steviol glycosides produced by the cell, and/or decreases the amount of one or more steviol glycosides produced by the cell. In some embodiments, the steviol glycoside-producing host further expresses a gene encoding a polypeptide capable of synthesizing GGPP from FPP and IPP; a gene encoding a polypeptide capable of synthesizing ent-copalyl diphosphate from GGPP; a gene encoding a polypeptide capable of synthesizing ent-kaurene from ent-copalyl diphosphate; a gene encoding a polypeptide capable of synthesizing ent-kaurenoic acid, ent-kaurenol, and/or ent-kaurenal from ent-kaurene; a gene encoding a polypeptide capable of reducing cytochrome P450 complex; and a gene encoding a polypeptide capable of synthesizing steviol from ent-kaurenoic acid; and/or a gene encoding a bifunctional polypeptide capable of synthesizing ent-copalyldiphosphate from GGPP and synthesizing ent-kaurene from ent-copalyl diphosphate.

In some aspects, the polypeptide capable of synthesizing geranylgeranyl pyrophosphate (GGPP) from farnesyl diphosphate (FPP) and isopentenyl diphosphate (IPP) comprises a polypeptide having an amino acid sequence set forth in SEQ ID NO:20 (which can be encoded by the nucleotide sequence set forth in SEQ ID NO:19), SEQ ID NO:22 (encoded by the nucleotide sequence set forth in SEQ ID NO:21), SEQ ID NO:24 (encoded by the nucleotide sequence set forth in SEQ ID NO:23), SEQ ID NO:26 (encoded by the nucleotide sequence set forth in SEQ ID NO:25), SEQ ID NO:28 (encoded by the nucleotide sequence set forth in SEQ ID NO:27), SEQ ID NO:30 (encoded by the nucleotide sequence set forth in SEQ ID NO:29), SEQ ID NO:32 (encoded by the nucleotide sequence set forth in SEQ ID NO:31), or SEQ ID NO:116 (encoded by the nucleotide sequence set forth in SEQ ID NO:115). In some embodiments, a recombinant host comprising a gene encoding a polypeptide capable of synthesizing geranylgeranyl pyrophosphate (GGPP) from farnesyl diphosphate (FPP) and isopentenyl diphosphate (IPP) further comprises one or more genes encoding one or more polypeptides capable of synthesizing UTP from UDP (e.g., a polypeptide having the amino acid sequence set forth in SEQ ID NO:123), one or more genes encoding one or more polypeptides capable of converting glucose-6-phosphate to glucose-1-phosphate (e.g., a polypeptide having the amino acid sequence set forth in SEQ ID NO:2, SEQ ID NO:119, SEQ ID NO:141, SEQ ID NO:143, SEQ ID NO:145, and/or SEQ ID NO:147), one or more genes encoding one or more polypeptides capable of debranching glycogen (e.g., a polypeptide having the amino acid sequence set forth in SEQ ID NO:157), one or more genes encoding one or more polypeptides capable of synthesizing glucose-1-phosphate from phosphate and glycogen (e.g., a polypeptide having the amino acid sequence set forth in SEQ ID NO:159), and/or one or more genes encoding one or more polypeptides capable of synthesizing UDP-glucose from UTP and glucose-1-phosphate (e.g., a polypeptide having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139). In some embodiments, the recombinant host is an S. cerevisiae host cell overexpressing one or more genes encoding one or more polypeptides involved in the UDP-glucose biosynthetic pathway (e.g., a polypeptide having the amino acid sequence set forth in SEQ ID NO:2, SEQ ID NO:119, SEQ ID NO:121, SEQ ID NO:123, SEQ ID NO:157, and/or SEQ ID NO:159).

In some aspects, the polypeptide capable of synthesizing ent-copalyl diphosphate from GGPP comprises a polypeptide having an amino acid sequence set forth in SEQ ID NO:34 (which can be encoded by the nucleotide sequence set forth in SEQ ID NO:33), SEQ ID NO:36 (encoded by the nucleotide sequence set forth in SEQ ID NO:35), SEQ ID NO:38 (encoded by the nucleotide sequence set forth in SEQ ID NO:37), SEQ ID NO:40 (encoded by the nucleotide sequence set forth in SEQ ID NO:39), or SEQ ID NO:42 (encoded by the nucleotide sequence set forth in SEQ ID NO:41). In some embodiments, the polypeptide capable of synthesizing ent-copalyl diphosphate from GGPP lacks a chloroplast transit peptide. In some embodiments, a recombinant host comprising a gene encoding a polypeptide capable of synthesizing ent-copalyl diphosphate from GGPP further comprises one or more genes encoding one or more polypeptides capable of synthesizing UTP from UDP (e.g., a polypeptide having the amino acid sequence set forth in SEQ ID NO:123), one or more genes encoding one or more polypeptides capable of converting glucose-6-phosphate to glucose-1-phosphate (e.g., a polypeptide having the amino acid sequence set forth in SEQ ID NO:2, SEQ ID NO:119, SEQ ID NO:141, SEQ ID NO:143, SEQ ID NO:145, and/or SEQ ID NO:147), one or more genes encoding one or more polypeptides capable of debranching glycogen (e.g., a polypeptide having the amino acid sequence set forth in SEQ ID NO:157), one or more genes encoding one or more polypeptides capable of synthesizing glucose-1-phosphate from phosphate and glycogen (e.g., a polypeptide having the amino acid sequence set forth in SEQ ID NO:159), and/or one or more genes encoding one or more polypeptides capable of synthesizing UDP-glucose from UTP and glucose-1-phosphate (e.g., a polypeptide having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139). In some embodiments, the recombinant host is an S. cerevisiae host cell overexpressing one or more genes encoding one or more polypeptides involved in the UDP-glucose biosynthetic pathway (e.g., a polypeptide having the amino acid sequence set forth in SEQ ID NO:2, SEQ ID NO:119, SEQ ID NO:121, SEQ ID NO:123, SEQ ID NO:157, and/or SEQ ID NO:159).

In some aspects, the polypeptide capable of synthesizing ent-kaurene from ent-copalyl diphosphate comprises a polypeptide having an amino acid sequence set forth in SEQ ID NO:44 (which can be encoded by the nucleotide sequence set forth in SEQ ID NO:43), SEQ ID NO:46 (encoded by the nucleotide sequence set forth in SEQ ID NO:45), SEQ ID NO:48 (encoded by the nucleotide sequence set forth in SEQ ID NO:47), SEQ ID NO:50 (encoded by the nucleotide sequence set forth in SEQ ID NO:49), or SEQ ID NO:52 (encoded by the nucleotide sequence set forth in SEQ ID NO:51). In some embodiments, a recombinant host comprising a gene encoding a polypeptide capable of synthesizing ent-kaurene from ent-copalyl diphosphate further comprises one or more genes encoding one or more polypeptides capable of synthesizing UTP from UDP (e.g., a polypeptide having the amino acid sequence set forth in SEQ ID NO:123), one or more genes encoding one or more polypeptides capable of converting glucose-6-phosphate to glucose-1-phosphate (e.g., a polypeptide having the amino acid sequence set forth in SEQ ID NO:2, SEQ ID NO:119, SEQ ID NO:141, SEQ ID NO:143, SEQ ID NO:145, and/or SEQ ID NO:147), one or more genes encoding one or more polypeptides capable of debranching glycogen (e.g., a polypeptide having the amino acid sequence set forth in SEQ ID NO:157), one or more genes encoding one or more polypeptides capable of synthesizing glucose-1-phosphate from phosphate and glycogen (e.g., a polypeptide having the amino acid sequence set forth in SEQ ID NO:159), and/or one or more genes encoding one or more polypeptides capable of synthesizing UDP-glucose from UTP and glucose-1-phosphate (e.g., a polypeptide having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139). In some embodiments, the recombinant host is an S. cerevisiae host cell overexpressing one or more genes encoding one or more polypeptides involved in the UDP-glucose biosynthetic pathway (e.g., a polypeptide having the amino acid sequence set forth in SEQ ID NO:2, SEQ ID NO:119, SEQ ID NO:121, SEQ ID NO:123, SEQ ID NO:157, and/or SEQ ID NO:159).

In some embodiments, a recombinant host comprises a gene encoding a bifunctional polypeptide capable of synthesizing ent-copalyl diphosphate from GGPP and synthesizing ent-kaurene from ent-copalyl diphosphate. In some aspects, the bifunctional polypeptide comprises a polypeptide having an amino acid sequence set forth in SEQ ID NO:54 (which can be encoded by the nucleotide sequence set forth in SEQ ID NO:53), SEQ ID NO:56 (encoded by the nucleotide sequence set forth in SEQ ID NO:55), or SEQ ID NO:58 (encoded by the nucleotide sequence set forth in SEQ ID NO:57). In some embodiments, a recombinant host comprising a gene encoding a bifunctional polypeptide capable of synthesizing ent-copalyl diphosphate from GGPP and synthesizing ent-kaurene from ent-copalyl diphosphate further comprises one or more genes encoding one or more polypeptides capable of synthesizing UTP from UDP (e.g., a polypeptide having the amino acid sequence set forth in SEQ ID NO:123), one or more genes encoding one or more polypeptides capable of converting glucose-6-phosphate to glucose-1-phosphate (e.g., a polypeptide having the amino acid sequence set forth in SEQ ID NO:2, SEQ ID NO:119, SEQ ID NO:141, SEQ ID NO:143, SEQ ID NO:145, and/or SEQ ID NO:147), one or more genes encoding one or more polypeptides capable of debranching glycogen (e.g., a polypeptide having the amino acid sequence set forth in SEQ ID NO:157), one or more genes encoding one or more polypeptides capable of synthesizing glucose-1-phosphate from phosphate and glycogen (e.g., a polypeptide having the amino acid sequence set forth in SEQ ID NO:159), and/or one or more genes encoding one or more polypeptides capable of synthesizing UDP-glucose from UTP and glucose-1-phosphate (e.g., a polypeptide having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139). In some embodiments, the recombinant host is an S. cerevisiae host cell overexpressing one or more genes encoding one or more polypeptides involved in the UDP-glucose biosynthetic pathway (e.g., a polypeptide having the amino acid sequence set forth in SEQ ID NO:2, SEQ ID NO:119, SEQ ID NO:121, SEQ ID NO:123, SEQ ID NO:157, and/or SEQ ID NO:159).

In some aspects, the polypeptide capable of synthesizing ent-kaurenoic acid, ent-kaurenol, and/or ent-kaurenal from ent-kaurene comprises a polypeptide having an amino acid sequence set forth in SEQ ID NO:60 (which can be encoded by the nucleotide sequence set forth in SEQ ID NO:59), SEQ ID NO:62 (encoded by the nucleotide sequence set forth in SEQ ID NO:61), SEQ ID NO:117 (encoded by the nucleotide sequence set forth in SEQ ID NO:63 or SEQ ID NO:64), SEQ ID NO:66 (encoded by the nucleotide sequence set forth in SEQ ID NO:65), SEQ ID NO:68 (encoded by the nucleotide sequence set forth in SEQ ID NO:67), SEQ ID NO:70 (encoded by the nucleotide sequence set forth in SEQ ID NO:69), SEQ ID NO:72 (encoded by the nucleotide sequence set forth in SEQ ID NO:71), SEQ ID NO:74 (encoded by the nucleotide sequence set forth in SEQ ID NO:73), or SEQ ID NO:76 (encoded by the nucleotide sequence set forth in SEQ ID NO:75). In some embodiments, a recombinant host comprising a gene encoding a polypeptide capable of synthesizing ent-kaurenoic acid, ent-kaurenol, and/or ent-kaurenal from ent-kaurene further comprises one or more genes encoding one or more polypeptides capable of synthesizing UTP from UDP (e.g., a polypeptide having the amino acid sequence set forth in SEQ ID NO:123), one or more genes encoding one or more polypeptides capable of converting glucose-6-phosphate to glucose-1-phosphate (e.g., a polypeptide having the amino acid sequence set forth in SEQ ID NO:2, SEQ ID NO:119, SEQ ID NO:141, SEQ ID NO:143, SEQ ID NO:145, and/or SEQ ID NO:147), one or more genes encoding one or more polypeptides capable of debranching glycogen (e.g., a polypeptide having the amino acid sequence set forth in SEQ ID NO:157), one or more genes encoding one or more polypeptides capable of synthesizing glucose-1-phosphate from phosphate and glycogen (e.g., a polypeptide having the amino acid sequence set forth in SEQ ID NO:159), and/or one or more genes encoding one or more polypeptides capable of synthesizing UDP-glucose from UTP and glucose-1-phosphate (e.g., a polypeptide having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139). In some embodiments, the recombinant host is an S. cerevisiae host cell overexpressing one or more genes encoding one or more polypeptides involved in the UDP-glucose biosynthetic pathway (e.g., a polypeptide having the amino acid sequence set forth in SEQ ID NO:2, SEQ ID NO:119, SEQ ID NO:121, SEQ ID NO:123, SEQ ID NO:157, and/or SEQ ID NO:159).

In some aspects, the polypeptide capable of reducing cytochrome P450 complex comprises a polypeptide having an amino acid sequence set forth in SEQ ID NO:78 (which can be encoded by the nucleotide sequence set forth in SEQ ID NO:77), SEQ ID NO:80 (encoded by the nucleotide sequence set forth in SEQ ID NO:79), SEQ ID NO:82 (encoded by the nucleotide sequence set forth in SEQ ID NO:81), SEQ ID NO:84 (encoded by the nucleotide sequence set forth in SEQ ID NO:83), SEQ ID NO:86 (encoded by the nucleotide sequence set forth in SEQ ID NO:85), SEQ ID NO:88 (encoded by the nucleotide sequence set forth in SEQ ID NO:87), SEQ ID NO:90 (encoded by the nucleotide sequence set forth in SEQ ID NO:89), or SEQ ID NO:92 (encoded by the nucleotide sequence set forth in SEQ ID NO:91). In some embodiments, a recombinant host comprising a gene encoding a polypeptide capable of reducing cytochrome P450 complex further comprises one or more genes encoding one or more polypeptides capable of synthesizing UTP from UDP (e.g., a polypeptide having the amino acid sequence set forth in SEQ ID NO:123), one or more genes encoding one or more polypeptides capable of converting glucose-6-phosphate to glucose-1-phosphate (e.g., a polypeptide having the amino acid sequence set forth in SEQ ID NO:2, SEQ ID NO:119, SEQ ID NO:141, SEQ ID NO:143, SEQ ID NO:145, and/or SEQ ID NO:147), one or more genes encoding one or more polypeptides capable of debranching glycogen (e.g., a polypeptide having the amino acid sequence set forth in SEQ ID NO:157), one or more genes encoding one or more polypeptides capable of synthesizing glucose-1-phosphate from phosphate and glycogen (e.g., a polypeptide having the amino acid sequence set forth in SEQ ID NO:159), and/or one or more genes encoding one or more polypeptides capable of synthesizing UDP-glucose from UTP and glucose-1-phosphate (e.g., a polypeptide having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139). In some embodiments, the recombinant host is an S. cerevisiae host cell overexpressing one or more genes encoding one or more polypeptides involved in the UDP-glucose biosynthetic pathway (e.g., a polypeptide having the amino acid sequence set forth in SEQ ID NO:2, SEQ ID NO:119, SEQ ID NO:121, SEQ ID NO:123, SEQ ID NO:157, and/or SEQ ID NO:159).

In some aspects, the polypeptide capable of synthesizing steviol from ent-kaurenoic acid comprises a polypeptide having an amino acid sequence set forth in SEQ ID NO:94 (which can be encoded by the nucleotide sequence set forth in SEQ ID NO:93), SEQ ID NO:97 (encoded by the nucleotide sequence set forth in SEQ ID NO:95 or SEQ ID NO:96), SEQ ID NO:100 (encoded by the nucleotide sequence set forth in SEQ ID NO:98 or SEQ ID NO:99), SEQ ID NO:101, SEQ ID NO:102, SEQ ID NO:103, SEQ ID NO:104, SEQ ID NO:106 (encoded by the nucleotide sequence set forth in SEQ ID NO:105), SEQ ID NO:108 (encoded by the nucleotide sequence set forth in SEQ ID NO:107), SEQ ID NO:110 (encoded by the nucleotide sequence set forth in SEQ ID NO:109), SEQ ID NO:112 (encoded by the nucleotide sequence set forth in SEQ ID NO:111), or SEQ ID NO:114 (encoded by the nucleotide sequence set forth in SEQ ID NO:113). In some embodiments, a recombinant host comprising a gene encoding a polypeptide capable of synthesizing steviol from ent-kaurenoic acid further comprises one or more genes encoding one or more polypeptides capable of synthesizing UTP from UDP (e.g., a polypeptide having the amino acid sequence set forth in SEQ ID NO:123), one or more genes encoding one or more polypeptides capable of converting glucose-6-phosphate to glucose-1-phosphate (e.g., a polypeptide having the amino acid sequence set forth in SEQ ID NO:2, SEQ ID NO:119, SEQ ID NO:141, SEQ ID NO:143, SEQ ID NO:145, and/or SEQ ID NO:147), one or more genes encoding one or more polypeptides capable of debranching glycogen (e.g., a polypeptide having the amino acid sequence set forth in SEQ ID NO:157), one or more genes encoding one or more polypeptides capable of synthesizing glucose-1-phosphate from phosphate and glycogen (e.g., a polypeptide having the amino acid sequence set forth in SEQ ID NO:159), and/or one or more genes encoding one or more polypeptides capable of synthesizing UDP-glucose from UTP and glucose-1-phosphate (e.g., a polypeptide having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139). In some embodiments, the recombinant host is an S. cerevisiae host cell overexpressing one or more genes encoding one or more polypeptides involved in the UDP-glucose biosynthetic pathway (e.g., a polypeptide having the amino acid sequence set forth in SEQ ID NO:2, SEQ ID NO:119, SEQ ID NO:121, SEQ ID NO:123, SEQ ID NO:157, and/or SEQ ID NO:159).

In some embodiments, a recombinant host comprises a nucleic acid encoding a polypeptide capable of glycosylating a steviol or a steviol glycoside at its C-13 hydroxyl group (e.g., UGT85C2 polypeptide) (SEQ ID NO:7), a nucleic acid encoding a polypeptide capable of beta 1,3 glycosylation of the C3′ of the 13-O-glucose, 19-O-glucose, or both 13-O-glucose and 19-O-glucose of a steviol glycoside (e.g., UGT76G1 polypeptide) (SEQ ID NO:9), a nucleic acid encoding a polypeptide capable of glycosylating the steviol or the steviol glycoside at its C-19 carboxyl group (e.g., UGT74G1 polypeptide) (SEQ ID NO:4), a nucleic acid encoding a polypeptide capable of beta 1,2 glycosylation of the C2′ of the 13-O-glucose, 19-O-glucose, or both 13-O-glucose and 19-O-glucose of a steviol glycoside (e.g., EUGT11 polypeptide) (SEQ ID NO:16). In some aspects, the polypeptide capable of beta 1,2 glycosylation of the C2′ of the 13-O-glucose, 19-O-glucose, or both 13-O-glucose and 19-O-glucose of a steviol glycoside (e.g., UGT91D2 polypeptide) can be a UGT91D2e polypeptide (SEQ ID NO:11) or a UGT91D2e-b polypeptide (SEQ ID NO:13). In some embodiments, a recombinant host comprising a gene encoding a polypeptide capable of glycosylating the steviol or the steviol glycoside further comprises one or more genes encoding one or more polypeptides capable of synthesizing UTP from UDP (e.g., a polypeptide having the amino acid sequence set forth in SEQ ID NO:123), one or more genes encoding one or more polypeptides capable of converting glucose-6-phosphate to glucose-1-phosphate (e.g., a polypeptide having the amino acid sequence set forth in SEQ ID NO:2, SEQ ID NO:119, SEQ ID NO:141, SEQ ID NO:143, SEQ ID NO:145, and/or SEQ ID NO:147), one or more genes encoding one or more polypeptides capable of debranching glycogen (e.g., a polypeptide having the amino acid sequence set forth in SEQ ID NO:157), one or more genes encoding one or more polypeptides capable of synthesizing glucose-1-phosphate from phosphate and glycogen (e.g., a polypeptide having the amino acid sequence set forth in SEQ ID NO:159), and/or one or more genes encoding one or more polypeptides capable of synthesizing UDP-glucose from UTP and glucose-1-phosphate (e.g., a polypeptide having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139). In some embodiments, the recombinant host is an S. cerevisiae host cell overexpressing one or more genes encoding one or more polypeptides involved in the UDP-glucose biosynthetic pathway (e.g., a polypeptide having the amino acid sequence set forth in SEQ ID NO:2, SEQ ID NO:119, SEQ ID NO:121, SEQ ID NO:123, SEQ ID NO:157, and/or SEQ ID NO:159).

In some aspects, the polypeptide capable of glycosylating a steviol or a steviol glycoside at its C-13 hydroxyl group is encoded by the nucleotide sequence set forth in SEQ ID NO:5 or SEQ ID NO:6, the polypeptide capable of beta 1,3 glycosylation of the C3′ of the 13-O-glucose, 19-O-glucose, or both 13-O-glucose and 19-O-glucose of a steviol glycoside is encoded by the nucleotide sequence set forth in SEQ ID NO:8, the polypeptide capable of glycosylating the steviol or the steviol glycoside at its C-19 carboxyl group is encoded by the nucleotide sequence set forth in SEQ ID NO:3, the polypeptide capable of beta 1,2 glycosylation of the C2′ of the 13-O-glucose, 19-O-glucose, or both 13-O-glucose and 19-O-glucose of a steviol glycoside is encoded by the nucleotide sequence set forth in SEQ ID NO:10, SEQ ID NO:12, SEQ ID NO:14, or SEQ ID NO:15. The skilled worker will appreciate that expression of these genes may be necessary to produce a particular steviol glycoside but that one or more of these genes can be endogenous to the host provided that at least one (and in some embodiments, all) of these genes is a recombinant gene introduced into the recombinant host.

In some embodiments, expression of a recombinant gene encoding a polypeptide capable of debranching glycogen and a recombinant gene encoding a polypeptide capable of synthesizing glucose-1-phosphate from phosphate and glycogen in a steviol glycoside-producing recombinant host increases the amount of one or more steviol glycosides, e.g., RebA, RebD, and/or RebM, produced by the cell by at least 10%, at least 25%, or at least 50%, at least 100%, at least 150%, at least 200%, or at least 250%, calculated as an increase in intracellular steviol glycoside concentration relative to a corresponding host lacking the one or more recombinant genes.

For example, in some embodiments, expression of a recombinant gene encoding a polypeptide capable of debranching glycogen (e.g., a polypeptide having the amino acid sequence set forth in SEQ ID NO:157) and a recombinant gene encoding a polypeptide capable of synthesizing glucose-1-phosphate from phosphate and glycogen (e.g., a polypeptide having the amino acid sequence set forth in SEQ ID NO:159) in a steviol glycoside-producing host increases the amount of one or more steviol glycosides, e.g., RebA, RebD, and/or RebM, produced by the cell by at least 10%, at least 25%, or at least 50%, at least 100%, at least 150%, at least 200%, or at least 250%, calculated as an increase in intracellular steviol glycoside concentration relative to a corresponding host lacking the one or more recombinant genes.

In some embodiments, expression of a recombinant gene encoding a polypeptide capable of synthesizing UTP from UDP, a recombinant gene encoding a polypeptide capable of converting glucose-6-phosphate to glucose-1-phosphate, a recombinant gene encoding a polypeptide capable of debranching glycogen, a recombinant gene encoding a polypeptide capable of synthesizing glucose-1-phosphate from phosphate and glycogen, and a recombinant gene encoding a polypeptide capable of synthesizing UDP-glucose from UTP and glucose-1-phosphate in a steviol glycoside-producing recombinant host increases the amount of one or more steviol glycosides, e.g., rubusoside, RebB, RebA, RebD, and/or RebM, produced by the cell by at least 10%, at least 25%, or at least 50%, at least 100%, at least 150%, at least 200%, or at least 250%, calculated as an increase in intracellular steviol glycoside concentration relative to a corresponding host lacking the one or more recombinant genes.

For example, in some embodiments, expression of a recombinant gene encoding a polypeptide capable of synthesizing UTP from UDP (e.g., a polypeptide having the amino acid sequence set forth in SEQ ID NO:123), a recombinant gene encoding a polypeptide capable of converting glucose-6-phosphate to glucose-1-phosphate (e.g. a polypeptide having the amino acid sequence set forth in SEQ ID NO:2, SEQ ID NO:119, SEQ ID NO:141, SEQ ID NO:143, SEQ ID NO:145, or SEQ ID NO:147), a recombinant gene encoding a polypeptide capable of debranching glycogen (e.g., a polypeptide having the amino acid sequence set forth in SEQ ID NO:157), a recombinant gene encoding a polypeptide capable of synthesizing glucose-1-phosphate from phosphate and glycogen (e.g., a polypeptide having the amino acid sequence set forth in SEQ ID NO:159), and a recombinant gene encoding a polypeptide capable of synthesizing UDP-glucose from UTP and glucose-1-phosphate (e.g., a polypeptide having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, or SEQ ID NO:139) in a steviol glycoside-producing host increases the amount of one or more steviol glycosides, e.g., rubusoside, RebB, RebA, RebD, and/or RebM, produced by the cell by at least 10%, at least 25%, or at least 50%, at least 100%, at least 150%, at least 200%, or at least 250%, calculated as an increase in intracellular steviol glycoside concentration relative to a corresponding host lacking the one or more recombinant genes.

In some embodiments, expression of a recombinant gene encoding a recombinant gene encoding a polypeptide capable of debranching glycogen and a recombinant gene encoding a polypeptide capable of synthesizing glucose-1-phosphate from phosphate and glycogen in a steviol glycoside-producing recombinant host decreases the amount of one or more steviol glycosides, e.g., 13-SMG, produced by the cell by at least 5%, e.g., at least 10%, or at least 15%, or at least 20%, or at least 25%, calculated as a decrease in intracellular steviol glycoside concentration relative to a corresponding steviol glycoside-producing host lacking the recombinant genes.

For example, in some embodiments, expression of a recombinant gene encoding a polypeptide capable of debranching glycogen having the amino acid sequence set forth in SEQ ID NO:157 and a recombinant gene encoding a polypeptide capable of synthesizing glucose-1-phosphate from phosphate and glycogen having the amino acid sequence set forth in SEQ ID NO:159 in a steviol glycoside-producing recombinant host decreases the amount of 13-SMG produced by the cell by at least 5%, e.g., at least 7.5%, or at least 10%, or at least 15%, or at least 20%, at least 25%, or at least 50%, calculated as decrease in intracellular 13-SMG concentration relative to a corresponding host lacking the one or more recombinant genes.

In some embodiments, expression of a recombinant gene encoding a polypeptide capable of synthesizing UTP from UDP, a recombinant gene encoding a polypeptide capable of converting glucose-6-phosphate to glucose-1-phosphate, a recombinant gene encoding a polypeptide capable of debranching glycogen, a recombinant gene encoding a polypeptide capable of synthesizing glucose-1-phosphate from phosphate and glycogen, and a recombinant gene encoding a polypeptide capable of synthesizing UDP-glucose from UTP and glucose-1-phosphate in a steviol glycoside-producing recombinant host decreases the amount of one or more steviol glycosides, e.g., 13-SMG and RebD, produced by the cell by at least 5%, e.g., at least 10%, or at least 15%, or at least 20%, or at least 25%, or at least 30%, or at least 35%, or at least 40%, or at least 45%, or at least 50%, calculated as a decrease in intracellular steviol glycoside concentration relative to a corresponding steviol glycoside-producing host lacking the recombinant genes.

For example, in some embodiments, expression of a recombinant gene encoding a polypeptide capable of synthesizing UTP from UDP having the amino acid sequence set forth in SEQ ID NO:123, a recombinant gene encoding a polypeptide capable of converting glucose-6-phosphate to glucose-1-phosphate having the amino acid sequence set forth in SEQ ID NO:2, a recombinant gene encoding a polypeptide capable of converting glucose-6-phosphate to glucose-1-phosphate having the amino acid sequence set forth in SEQ ID NO:119, a recombinant gene encoding a polypeptide capable of debranching glycogen having the amino acid sequence set forth in SEQ ID NO:157, a recombinant gene encoding a polypeptide capable of synthesizing glucose-1-phosphate from phosphate and glycogen having the amino acid sequence set forth in SEQ ID NO:159, a recombinant gene encoding a polypeptide capable of synthesizing UDP-glucose from UTP and glucose-1-phosphate having the amino acid sequence set forth in SEQ ID NO:121, and further expression of a recombinant gene encoding a polypeptide capable of synthesizing UDP-glucose from UTP and glucose-1-phosphate having the amino acid sequence set forth in, e.g., SEQ ID NO:127, SEQ ID NO:133, SEQ ID NO:129, SEQ ID NO:125, SEQ ID NO:139, or SEQ ID NO:135, in a steviol glycoside-producing recombinant host decreases the amount of 13-SMG produced by the cell by at least 5%, e.g., at least 7.5%, or at least 10%, or at least 15%, or at least 20%, or at least 25%, or at least 30%, at least 35%, or at least 50%, calculated as a decrease in intracellular 13-SMG concentration relative to a corresponding host lacking the one or more recombinant genes.

In some embodiments, expression of a recombinant gene encoding a polypeptide capable of synthesizing UTP from UDP, a recombinant gene encoding a polypeptide capable of converting glucose-6-phosphate to glucose-1-phosphate, a recombinant gene encoding a polypeptide capable of debranching glycogen, a recombinant gene encoding a polypeptide capable of synthesizing glucose-1-phosphate from phosphate and glycogen, and a recombinant gene encoding a polypeptide capable of synthesizing UDP-glucose from UTP and glucose-1-phosphate in a steviol glycoside-producing recombinant host increases the total amount of steviol glycosides (i.e., the total amount of mono-, di-, tri-, tetra-penta-, hexa-, and hepta-glycosylated steviol compounds) by at least 5%, e.g., at least 7.5%, or at least 10%, or at least 12.5%, or at least 15%, or at least 17.5%, or at least 20%, or at least 25%, or at least 27.5%, or at least 30%, or at least 35%, calculated as an increase in intracellular steviol glycoside concentration relative to a corresponding steviol glycoside-producing host lacking the recombinant genes.

For example, in some embodiments, expression of a recombinant gene encoding a polypeptide capable of synthesizing UTP from UDP having the amino acid sequence set forth in SEQ ID NO:123, a recombinant gene encoding a polypeptide capable of converting glucose-6-phosphate to glucose-1-phosphate having the amino acid sequence set forth in SEQ ID NO:2, a recombinant gene encoding a polypeptide capable of converting glucose-6-phosphate to glucose-1-phosphate having the amino acid sequence set forth in SEQ ID NO:119, a recombinant gene encoding a polypeptide capable of debranching glycogen having the amino acid sequence set forth in SEQ ID NO:157, a recombinant gene encoding a polypeptide capable of synthesizing glucose-1-phosphate from phosphate and glycogen having the amino acid sequence set forth in SEQ ID NO:159, a recombinant gene encoding a polypeptide capable of synthesizing UDP-glucose from UTP and glucose-1-phosphate having the amino acid sequence set forth in SEQ ID NO:121, and further expression of a recombinant gene encoding a polypeptide capable of synthesizing UDP-glucose from UTP and glucose-1-phosphate having the amino acid sequence set forth in, e.g., SEQ ID NO:133, SEQ ID NO:129, SEQ ID NO:131, SEQ ID NO:125, SEQ ID NO:139, or SEQ ID NO:135, in a steviol glycoside-producing recombinant host increases the total amount of steviol glycosides (i.e., the total amount of mono-, di-, tri-, tetra-penta-, hexa-, and hepta-glycosylated steviol compounds) by at least 5%, e.g., at least 7.5%, or at least 10%, or at least 12.5%, or at least 15%, or at least 17.5%, or at least 20%, or at least 25%, or at least 27.5%, or at least 30%, or at least 35%, calculated as an increase in intracellular steviol glycoside concentration relative to a corresponding steviol glycoside-producing host lacking the recombinant genes.

In some other embodiments, the total amount of steviol glycosides produced by a steviol glycoside-producing recombinant host cell is unchanged (i.e., increased or decreased by less than 5%, or less than 4%, or less than 3%, or less than 2%, or less than 1%) by expression in the host of a recombinant gene encoding a polypeptide capable of synthesizing UTP from UDP, a recombinant gene encoding a polypeptide capable of converting glucose-6-phosphate to glucose-1-phosphate, a recombinant gene encoding a polypeptide capable of debranching glycogen, a recombinant gene encoding a polypeptide capable of synthesizing glucose-1-phosphate from phosphate and glycogen, and/or a recombinant gene encoding a polypeptide capable of synthesizing UDP-glucose from UTP and glucose-1-phosphate.

For example, in some embodiments, expression of a recombinant gene encoding a polypeptide capable of debranching glycogen having the amino acid sequence set forth in SEQ ID NO:157 and a recombinant gene encoding a polypeptide capable of synthesizing glucose-1-phosphate from phosphate and glycogen having the amino acid sequence set forth in SEQ ID NO:159 in a steviol glycoside-producing recombinant host increases the total amount of steviol glycosides produced by the host by less than 5%, e.g., less than 4%, or less than 3%, or less than 2%.

In another example, in some embodiments, expression of a recombinant gene encoding a polypeptide capable of synthesizing UTP from UDP having the amino acid sequence set forth in SEQ ID NO:123, a recombinant gene encoding a polypeptide capable of converting glucose-6-phosphate to glucose-1-phosphate having the amino acid sequence set forth in SEQ ID NO:2, a recombinant gene encoding a polypeptide capable of converting glucose-6-phosphate to glucose-1-phosphate having the amino acid sequence set forth in SEQ ID NO:119, a recombinant gene encoding a polypeptide capable of debranching glycogen having the amino acid sequence set forth in SEQ ID NO:157, a recombinant gene encoding a polypeptide capable of synthesizing glucose-1-phosphate from phosphate and glycogen having the amino acid sequence set forth in SEQ ID NO:159, and a recombinant gene encoding a polypeptide capable of synthesizing UDP-glucose from UTP and glucose-1-phosphate having the amino acid sequence set forth in SEQ ID NO:121 in a steviol glycoside-producing recombinant host increases the total amount of steviol glycosides produced by the host by less than 5%, e.g., less than 4%, or less than 3%, or less than 2%.

The person of ordinary skill in the art will appreciate that, in such embodiments, expression of one or more genes encoding a polypeptide involved in the involved in the UDP-glucose biosynthetic pathway may affect the relative levels of steviol glycosides produced by the recombinant host, e.g., by increasing the level of UDP-glucose available as a substrate for a polypeptide capable of glycosylating a steviol or a steviol glycoside.

For example, in some embodiments, expression of a recombinant gene encoding a polypeptide capable of debranching glycogen having the amino acid sequence set forth in SEQ ID NO:157 and a recombinant gene encoding a polypeptide capable of synthesizing glucose-1-phosphate from phosphate and glycogen having the amino acid sequence set forth in SEQ ID NO:159 in a steviol glycoside-producing recombinant host increases the total amount of steviol glycosides produced by the host by less than 5%, e.g., less than 4%, or less than 3%, or less than 2%, increases the amount of RebA, RebD, and/or RebM produced by the host by at least 10%, at least 25%, or at least 50%, at least 100%, at least 150%, at least 200%, or at least 250%, calculated as an increase in intracellular steviol glycoside concentration relative to a corresponding host lacking the one or more recombinant genes and decreases the amount of 13-SMG produced by the host cell by at least 5%, e.g., at least 10%, at least 20%, at least 25%, or at least 50%, calculated as a decrease in intracellular 13-SMG concentration relative to a corresponding host lacking the one or more recombinant genes.

In another example, in some embodiments, expression of a recombinant gene encoding a polypeptide capable of synthesizing UTP from UDP having the amino acid sequence set forth in SEQ ID NO:123, a recombinant gene encoding a polypeptide capable of converting glucose-6-phosphate to glucose-1-phosphate having the amino acid sequence set forth in SEQ ID NO:2, a recombinant gene encoding a polypeptide capable of converting glucose-6-phosphate to glucose-1-phosphate having the amino acid sequence set forth in SEQ ID NO:119, a recombinant gene encoding a polypeptide capable of debranching glycogen having the amino acid sequence set forth in SEQ ID NO:157, a recombinant gene encoding a polypeptide capable of synthesizing glucose-1-phosphate from phosphate and glycogen having the amino acid sequence set forth in SEQ ID NO:159, and a recombinant gene encoding a polypeptide capable of synthesizing UDP-glucose from UTP and glucose-1-phosphate having the amino acid sequence set forth in SEQ ID NO:121 in a steviol glycoside-producing recombinant host increases the total amount of steviol glycosides produced by the host by less than 5%, e.g., less than 4%, or less than 3%, or less than 2%, increases the amount of RebM produced by the host by at least 10%, at least 25%, or at least 50%, at least 100%, at least 150%, at least 200%, or at least 250%, calculated as an increase in intracellular RebM concentration relative to a corresponding host lacking the one or more recombinant genes, and decreases the amount of RebD produced by the host by at least 10%, e.g., at least 20%, or at least 30%, at least 40%, or at least 50%, calculated as a decrease in intracellular RebD concentration relative to a corresponding host lacking the one or more recombinant genes.

In some embodiments, a recombinant host cell comprises one or more genes encoding one or more polypeptides capable of debranching glycogen (e.g., a polypeptide having the amino acid sequence set forth in SEQ ID NO:157) and/or one or more genes encoding one or more polypeptides capable of synthesizing glucose-1-phosphate from phosphate and glycogen (e.g., a polypeptide having the amino acid sequence set forth in SEQ ID NO:159). In some embodiments, a recombinant host cell comprises one or more genes encoding one or more polypeptides capable of synthesizing UTP from UDP (e.g., a polypeptide having the amino acid sequence set forth in SEQ ID NO:123), one or more genes encoding one or more polypeptides capable of converting glucose-6-phosphate to glucose-1-phosphate (e.g., a polypeptide having the amino acid sequence set forth in SEQ ID NO:2, SEQ ID NO:119, SEQ ID NO:141, SEQ ID NO:143, SEQ ID NO:145, and/or SEQ ID NO:147), one or more genes encoding one or more polypeptides capable of debranching glycogen (e.g., a polypeptide having the amino acid sequence set forth in SEQ ID NO:157), one or more genes encoding one or more polypeptides capable of synthesizing glucose-1-phosphate from phosphate and glycogen (e.g., a polypeptide having the amino acid sequence set forth in SEQ ID NO:159), and/or one or more genes encoding one or more polypeptides capable of synthesizing UDP-glucose from UTP and glucose-1-phosphate (e.g., a polypeptide having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139).

In certain embodiments, a recombinant host comprises one or more recombinant genes having a nucleotide sequence native to the host that encode one or more polypeptides capable of synthesizing UTP from UDP, one or more polypeptides capable of converting glucose-6-phosphate to glucose-1-phosphate, one or more polypeptides capable of debranching glycogen, one or more polypeptides capable of synthesizing glucose-1-phosphate from phosphate and glycogen, and/or one or more polypeptides capable of synthesizing UDP-glucose from UTP and glucose-1-phosphate, i.e., a recombinant host overexpresses one or more polypeptides capable of synthesizing UTP from UDP, one or more polypeptides capable of converting glucose-6-phosphate to glucose-1-phosphate, one or more polypeptides capable of debranching glycogen, one or more polypeptides capable of synthesizing glucose-1-phosphate from phosphate and glycogen, and/or one or more polypeptides capable of synthesizing UDP-glucose from UTP and glucose-1-phosphate.

In certain such embodiments, a recombinant host cell overexpresses one or more genes encoding one or more polypeptides capable of synthesizing UTP from UDP (e.g., an S. cerevisiae host cell expressing a recombinant gene encoding a polypeptide having the amino acid sequence set forth in SEQ ID NO:123), one or more genes encoding one or more polypeptides capable of converting glucose-6-phosphate to glucose-1-phosphate (e.g., an S. cerevisiae host cell expressing a recombinant gene encoding a polypeptide having the amino acid sequence set forth in SEQ ID NO:2, and/or SEQ ID NO:119), one or more genes encoding one or more polypeptides capable of debranching glycogen (e.g., an S. cerevisiae host cell expressing a recombinant gene encoding a polypeptide having the amino acid sequence set forth in SEQ ID NO:157), one or more genes encoding one or more polypeptides capable of synthesizing glucose-1-phosphate from phosphate and glycogen (e.g., an S. cerevisiae host cell expressing a recombinant gene encoding a polypeptide having the amino acid sequence set forth in SEQ ID NO:159), and/or one or more genes encoding one or more polypeptides capable of synthesizing UDP-glucose from UTP and glucose-1-phosphate (e.g., an S. cerevisiae host cell expressing a recombinant gene encoding a polypeptide having the amino acid sequence set forth in SEQ ID NO:121).

In one example, a recombinant S. cerevisiae host cell overexpresses a gene encoding a polypeptide having the amino acid sequence set forth in SEQ ID NO:157 and a gene encoding a polypeptide having the amino acid sequence set forth in SEQ ID NO:159. In another example, a recombinant S. cerevisiae host cell overexpresses a gene encoding a polypeptide having the amino acid sequence set forth in SEQ ID NO:123, a gene encoding a polypeptide having the amino acid sequence set forth in SEQ ID NO:2, a gene encoding a polypeptide having the amino acid sequence set forth in SEQ ID NO:119, a gene encoding a polypeptide having the amino acid sequence set forth in SEQ ID NO:121, a gene encoding a polypeptide having the amino acid sequence set forth in SEQ ID NO:157, and a gene encoding a polypeptide having the amino acid sequence set forth in SEQ ID NO:159.

In certain embodiments, a recombinant host cell comprising one or more genes encoding one or more polypeptides capable of synthesizing UTP from UDP (e.g., a polypeptide having the amino acid sequence set forth in SEQ ID NO:123), one or more genes encoding one or more polypeptides capable of converting glucose-6-phosphate to glucose-1-phosphate (e.g., a polypeptide having the amino acid sequence set forth in SEQ ID NO:2, SEQ ID NO:119, SEQ ID NO:141, SEQ ID NO:143, SEQ ID NO:145, and/or SEQ ID NO:147), one or more genes encoding one or more polypeptides capable of debranching glycogen (e.g., a polypeptide having the amino acid sequence set forth in SEQ ID NO:157), one or more genes encoding one or more polypeptides capable of synthesizing glucose-1-phosphate from phosphate and glycogen (e.g., a polypeptide having the amino acid sequence set forth in SEQ ID NO:159), and/or one or more genes encoding one or more polypeptides capable of synthesizing UDP-glucose from UTP and glucose-1-phosphate (e.g., a polypeptide having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139), further comprises a gene encoding a polypeptide capable of glycosylating a steviol or a steviol glycoside at its C-13 hydroxyl group (e.g., a polypeptide having the amino acid sequence set forth in SEQ ID NO:7); a gene encoding a polypeptide capable of beta 1,3 glycosylation of the C3′ of the 13-O-glucose, 19-O-glucose, or both 13-O-glucose and 19-O-glucose of a steviol glycoside (e.g., a polypeptide having the amino acid sequence set forth in SEQ ID NO:9); a gene encoding a polypeptide capable of glycosylating the steviol or the steviol glycoside at its C-19 carboxyl group (e.g., a polypeptide having the amino acid sequence set forth in SEQ ID NO:4); and/or a gene encoding a polypeptide capable of beta 1,2 glycosylation of the C2′ of the 13-O-glucose, 19-O-glucose, or both 13-O-glucose and 19-O-glucose of a steviol glycoside (e.g., a polypeptide having the amino acid sequence set forth in SEQ ID NO:11, SEQ ID NO:13, or SEQ ID NO:16). In certain such embodiments, the recombinant host cell further comprises a gene encoding a polypeptide capable of synthesizing GGPP from FPP and IPP (e.g., a polypeptide having the amino acid sequence set forth in SEQ ID NO:20); a gene encoding a polypeptide capable of synthesizing ent-copalyl diphosphate from GGPP (e.g., a polypeptide having the amino acid sequence set forth in SEQ ID NO:40); a gene encoding a polypeptide capable of synthesizing ent-kaurene from ent-copalyl diphosphate (e.g., a polypeptide having the amino acid sequence set forth in SEQ ID NO:52); a gene encoding a polypeptide capable of synthesizing ent-kaurenoic acid, ent-kaurenol, and/or ent-kaurenal from ent-kaurene (e.g., a polypeptide having the amino acid sequence set forth in SEQ ID NO:60 or SEQ ID NO:117); a gene encoding a polypeptide capable of reducing cytochrome P450 complex (e.g., a polypeptide having the amino acid sequence set forth in SEQ ID NO:78, SEQ ID NO:86, or SEQ ID NO:92); and/or a gene encoding a polypeptide capable of synthesizing steviol from ent-kaurenoic acid (e.g., a polypeptide having the amino acid sequence set forth in SEQ ID NO:94).

In some embodiments, a recombinant host comprises two or more genes encoding two or more polypeptides capable of converting glucose-6-phosphate to glucose-1-phosphate (e.g., two or more polypeptides having the amino acid sequence set forth in SEQ ID NO:2, SEQ ID NO:119, SEQ ID NO:141, SEQ ID NO:143, SEQ ID NO:145, and/or SEQ ID NO:147), and/or two or more genes encoding two or more polypeptides capable of synthesizing UDP-glucose from UTP and glucose-1-phosphate (e.g., two or more polypeptides having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139).

In certain such embodiments, a recombinant host comprises two or more genes encoding two or more polypeptides capable of converting glucose-6-phosphate to glucose-1-phosphate, e.g., two or more genes encoding two or more polypeptides having the amino acid sequence set forth in SEQ ID NO:2, SEQ ID NO:119, SEQ ID NO:141, SEQ ID NO:143, SEQ ID NO:145, and/or SEQ ID NO:147. In one example, a recombinant host comprises a gene encoding a polypeptide having the amino acid sequence set forth in SEQ ID NO:2 and a polypeptide having the amino acid sequence set forth in SEQ ID NO:119. In another example, a recombinant host comprises a gene encoding a polypeptide having the amino acid sequence set forth in SEQ ID NO:2, a polypeptide having the amino acid sequence set forth in SEQ ID NO:119, and a polypeptide having the amino acid sequence set forth in SEQ ID NO:145. In some embodiments, the recombinant host further comprises a gene encoding a polypeptide capable of synthesizing UTP from UDP (e.g., a polypeptide having the amino acid sequence set forth in SEQ ID NO:123), a gene encoding a polypeptide capable of debranching glycogen (e.g., a polypeptide having the amino acid sequence set forth in SEQ ID NO:157), a gene encoding a polypeptide capable of synthesizing glucose-1-phosphate from phosphate and glycogen (e.g., a polypeptide having the amino acid sequence set forth in SEQ ID NO:159), and/or one or more genes encoding one or more polypeptides capable of synthesizing UDP-glucose from UTP and glucose-1-phosphate (e.g., a polypeptide having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139).

In certain such embodiments, a recombinant host comprises two or more genes encoding two or more polypeptides capable of synthesizing UDP-glucose from UTP and glucose-1-phosphate, e.g., two or more genes encoding two or more polypeptides having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139. In one example, a recombinant host comprises a gene encoding a polypeptide having the amino acid sequence set forth in SEQ ID NO:121 and a polypeptide having the amino acid sequence set forth in SEQ ID NO:125. In another example, a recombinant host comprises a gene encoding a polypeptide having the amino acid sequence set forth in SEQ ID NO:121 and a polypeptide having the amino acid sequence set forth in SEQ ID NO:127. In another example, a recombinant host comprises a gene encoding a polypeptide having the amino acid sequence set forth in SEQ ID NO:121 and a polypeptide having the amino acid sequence set forth in SEQ ID NO:129. In another example, a recombinant host comprises a gene encoding a polypeptide having the amino acid sequence set forth in SEQ ID NO:121 and a polypeptide having the amino acid sequence set forth in SEQ ID NO:131. In another example, a recombinant host comprises a gene encoding a polypeptide having the amino acid sequence set forth in SEQ ID NO:121 and a gene encoding a gene encoding a polypeptide having the amino acid sequence set forth in SEQ ID NO:133. In another example, a recombinant host comprises a gene encoding a polypeptide having the amino acid sequence set forth in SEQ ID NO:121 and a gene encoding a polypeptide having the amino acid sequence set forth in SEQ ID NO:135. In another example, a recombinant host comprises a gene encoding a polypeptide having the amino acid sequence set forth in SEQ ID NO:121 and a gene encoding a polypeptide having the amino acid sequence set forth in SEQ ID NO:137. In another example, a recombinant host comprises a gene encoding a polypeptide having the amino acid sequence set forth in SEQ ID NO:121 and a gene encoding a polypeptide having the amino acid sequence set forth in SEQ ID NO:139. In some embodiments, the recombinant host further comprises a gene encoding a polypeptide capable of synthesizing UTP from UDP (e.g., a polypeptide having the amino acid sequence set forth in SEQ ID NO:123), a gene encoding a polypeptide capable of debranching glycogen (e.g., a polypeptide having the amino acid sequence set forth in SEQ ID NO:157), a gene encoding a polypeptide capable of synthesizing glucose-1-phosphate from phosphate and glycogen (e.g., a polypeptide having the amino acid sequence set forth in SEQ ID NO:159), and/or one or more genes encoding one or more polypeptides capable of converting glucose-6-phosphate to glucose-1-phosphate (e.g., one or more polypeptides having the amino acid sequence set forth in SEQ ID NO:2, SEQ ID NO:119, SEQ ID NO:141, SEQ ID NO:143, SEQ ID NO:145, and/or SEQ ID NO:147).

In certain such embodiments, a recombinant host comprising two or more genes encoding two or more polypeptides capable of converting glucose-6-phosphate to glucose-1-phosphate (e.g., two or more polypeptides having the amino acid sequence set forth in SEQ ID NO:2, SEQ ID NO:119, SEQ ID NO:141, SEQ ID NO:143, SEQ ID NO:145, and/or SEQ ID NO:147), and/or two or more genes encoding two or more polypeptides capable of synthesizing UDP-glucose from UTP and glucose-1-phosphate (e.g., two or more polypeptides having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139) is a host cell overexpressing one or more genes encoding one or more polypeptides involved in the UDP-glucose biosynthetic pathway (e.g., an S. cerevisiae host cell expressing one or more genes encoding one or more polypeptides having the amino acid sequence set forth in SEQ ID NO:2, SEQ ID NO:119, SEQ ID NO:121, SEQ ID NO:123, SEQ ID NO:157, and/or SEQ ID NO:159).

In certain embodiments, a recombinant host cell comprising two or more genes encoding two or more polypeptides capable of converting glucose-6-phosphate to glucose-1-phosphate (e.g., two or more polypeptides having the amino acid sequence set forth in SEQ ID NO:2, SEQ ID NO:119, SEQ ID NO:141, SEQ ID NO:143, SEQ ID NO:145, and/or SEQ ID NO:147), and/or two or more genes encoding two or more polypeptides capable of synthesizing UDP-glucose from UTP and glucose-1-phosphate (e.g., two or more polypeptides having the amino acid sequence set forth in SEQ ID NO:121, SEQ ID NO:125, SEQ ID NO:127, SEQ ID NO:129, SEQ ID NO:131, SEQ ID NO:133, SEQ ID NO:135, SEQ ID NO:137, and/or SEQ ID NO:139), further comprises a gene encoding polypeptide capable of synthesizing UTP from UDP (e.g., a polypeptide having the amino acid sequence set forth in SEQ ID NO:123), a gene encoding a polypeptide capable of debranching glycogen (e.g., a polypeptide having the amino acid sequence set forth in SEQ ID NO:157), a gene encoding a polypeptide capable of synthesizing glucose-1-phosphate from phosphate and glycogen (e.g., a polypeptide having the amino acid sequence set forth in SEQ ID NO:159), a gene encoding a polypeptide capable of glycosylating a steviol or a steviol glycoside at its C-13 hydroxyl group (e.g., a polypeptide having the amino acid sequence set forth in SEQ ID NO:7); a gene encoding a polypeptide capable of beta 1,3 glycosylation of the C3′ of the 13-O-glucose, 19-O-glucose, or both 13-O-glucose and 19-O-glucose of a steviol glycoside (e.g., a polypeptide having the amino acid sequence set forth in SEQ ID NO:9); a gene encoding a polypeptide capable of glycosylating the steviol or the steviol glycoside at its C-19 carboxyl group (e.g., a polypeptide having the amino acid sequence set forth in SEQ ID NO:4); and/or a gene encoding a polypeptide capable of beta 1,2 glycosylation of the C2′ of the 13-O-glucose, 19-O-glucose, or both 13-O-glucose and 19-O-glucose of a steviol glycoside (e.g., a polypeptide having the amino acid sequence set forth in SEQ ID NO:11, SEQ ID NO:13, or SEQ ID NO:16). In certain such embodiments, the recombinant host cell further comprises a gene encoding a polypeptide capable of synthesizing GGPP from FPP and IPP (e.g., a polypeptide having the amino acid sequence set forth in SEQ ID NO:20); a gene encoding a polypeptide capable of synthesizing ent-copalyl diphosphate from GGPP (e.g., a polypeptide having the amino acid sequence set forth in SEQ ID NO:40); a gene encoding a polypeptide capable of synthesizing ent-kaurene from ent-copalyl diphosphate (e.g., a polypeptide having the amino acid sequence set forth in SEQ ID NO:52); a gene encoding a polypeptide capable of synthesizing ent-kaurenoic acid, ent-kaurenol, and/or ent-kaurenal from ent-kaurene (e.g., a polypeptide having the amino acid sequence set forth in SEQ ID NO:60 or SEQ ID NO:117); a gene encoding a polypeptide capable of reducing cytochrome P450 complex (e.g., a polypeptide having the amino acid sequence set forth in SEQ ID NO:78, SEQ ID NO:86, or SEQ ID NO:92); and/or a gene encoding a polypeptide capable of synthesizing steviol from ent-kaurenoic acid (e.g., a polypeptide having the amino acid sequence set forth in SEQ ID NO:94).

In some embodiments, one or more steviol glycosides or a steviol glycoside composition is produced in an in vitro method, comprising adding a polypeptide capable of debranching glycogen comprises a polypeptide having at least 60% sequence identity to the amino acid sequence set forth in SEQ ID NO:157 and/or a polypeptide capable of synthesizing glucose-1-phosphate comprises a polypeptide having at least 55% sequence identity to the amino acid sequence set forth in SEQ ID NO:159; and, optionally, one or more of: a polypeptide capable of synthesizing UTP from UDP comprises a polypeptide having at least 60% sequence identity to the amino acid sequence set forth in SEQ ID NO:123; a polypeptide capable of converting glucose-6-phosphate to glucose-1-phosphate comprises a polypeptide having at least 60% sequence identity to the amino acid sequence set forth in any one of SEQ ID NOs:2, 119, or 143 or a polypeptide having at least 55% sequence identity to the amino acid sequence set forth in any one of SEQ ID NOs:141, 145, or 147; and/or a polypeptide capable of synthesizing UDP-glucose from UTP and glucose-1-phosphate comprises a polypeptide having at least 60% sequence identity to the amino acid sequence set forth in any one of SEQ ID NOs:121 or 127, a polypeptide having at least 55% sequence identity to the amino acid sequence set forth in any one of SEQ ID NOs:125, 129, 133, 135, 137, or 139 or a polypeptide having at least 70% sequence identity to the amino acid sequence set forth in SEQ ID NO:131; and one or more of: a polypeptide capable of glycosylating the steviol or the steviol glycoside at its C-13 hydroxyl group thereof comprises a polypeptide having at least 55% sequence identity to the amino acid sequence set forth in SEQ ID NO:7; a polypeptide capable of beta 1,3 glycosylation of the C3′ of the 13-O-glucose, 19-O-glucose, or both 13-O-glucose and 19-O-glucose of the steviol glycoside comprises a polypeptide having at least 50% sequence identity to the amino acid sequence set forth in SEQ ID NO:9; a polypeptide capable of glycosylating the steviol or the steviol glycoside at its C-19 carboxyl group thereof comprises a polypeptide having at least 55% sequence identity to the amino acid sequence set forth in SEQ ID NO:4; a polypeptide capable of beta 1,2 glycosylation of the C2′ of the 13-O-glucose, 19-O-glucose, or both 13-O-glucose and 19-O-glucose of the steviol glycoside comprises a polypeptide having at least 80% sequence identity to the amino acid sequence set forth in SEQ ID NO:11; a polypeptide having at least 80% sequence identity to the amino acid sequence set forth in SEQ ID NO:13; or a polypeptide having at least 65% sequence identity to the amino acid sequence set forth in SEQ ID NO:16; and a plant-derived or synthetic steviol, steviol precursors, and/or steviol glycosides to a reaction mixture; wherein at least one of the polypeptide is a recombinant polypeptide; and producing the one or more steviol glycosides or the steviol glycoside composition thereby.

In one aspect of the in vitro methods disclosed herein, the reaction mixture comprises: (a) one or more steviol glycosides or steviol glycoside composition; (b) a polypeptide capable of debranching glycogen having at least 60% sequence identity to the amino acid sequence set forth in SEQ ID NO:157 and/or a polypeptide capable of synthesizing glucose-1-phosphate comprises a polypeptide having at least 55% sequence identity to the amino acid sequence set forth in SEQ ID NO:159; and, optionally, one or more of: a polypeptide capable of synthesizing UTP from UDP comprises a polypeptide having at least 60% sequence identity to the amino acid sequence set forth in SEQ ID NO:123; a polypeptide capable of converting glucose-6-phosphate to glucose-1-phosphate comprises a polypeptide having at least 60% sequence identity to the amino acid sequence set forth in any one of SEQ ID NOs:2, 119, or 143 or a polypeptide having at least 55% sequence identity to the amino acid sequence set forth in any one of SEQ ID NOs:141, 145, or 147; and/or a polypeptide capable of synthesizing UDP-glucose from UTP and glucose-1-phosphate comprises a polypeptide having at least 60% sequence identity to the amino acid sequence set forth in any one of SEQ ID NOs:121 or 127, a polypeptide having at least 55% sequence identity to the amino acid sequence set forth in any one of SEQ ID NOs:125, 129, 133, 135, 137, or 139 or a polypeptide having at least 70% sequence identity to the amino acid sequence set forth in SEQ ID NO:131; and one or more of: a polypeptide capable of glycosylating the steviol or the steviol glycoside at its C-13 hydroxyl group thereof comprises a polypeptide having at least 55% sequence identity to the amino acid sequence set forth in SEQ ID NO:7; a polypeptide capable of beta 1,3 glycosylation of the C3′ of the 13-O-glucose, 19-O-glucose, or both 13-O-glucose and 19-O-glucose of the steviol glycoside comprises a polypeptide having at least 50% sequence identity to the amino acid sequence set forth in SEQ ID NO:9; a polypeptide capable of glycosylating the steviol or the steviol glycoside at its C-19 carboxyl group thereof comprises a polypeptide having at least 55% sequence identity to the amino acid sequence set forth in SEQ ID NO:4; a polypeptide capable of beta 1,2 glycosylation of the C2′ of the 13-O-glucose, 19-O-glucose, or both 13-O-glucose and 19-O-glucose of the steviol glycoside comprises a polypeptide having at least 80% sequence identity to the amino acid sequence set forth in SEQ ID NO:11; a polypeptide having at least 80% sequence identity to the amino acid sequence set forth in SEQ ID NO:13; or a polypeptide having at least 65% sequence identity to the amino acid sequence set forth in SEQ ID NO:16; (c) uridine diphosphate (UDP)-glucose, UDP-rhamnose, UDP-xylose, and/or N-acetyl-glucosamine ; and/or (d) reaction buffer and/or salts.

In one aspect of the in vitro methods disclosed herein, the one or more steviol glycosides is, or the steviol glycoside composition comprises, steviol-13-O-glucoside (13-SMG), steviol-1,2-Bioside, steviol-1,3-Bioside, steviol-19-O-glucoside (19-SMG), 1,2-stevioside, 1,3-stevioside (RebG), rubusoside, rebaudioside A (RebA), rebaudioside B (RebB), rebaudioside C (RebC), rebaudioside D (RebD), rebaudioside E (RebE), rebaudioside F (RebF), rebaudioside M (RebM), rebaudioside Q (RebQ), rebaudioside I (RebI), dulcoside A, and/or an isomer thereof.

In some embodiments, one or more steviol glycosides or a steviol glycoside composition is produced by whole cell bioconversion. For whole cell bioconversion to occur, a host cell expressing one or more enzymes involved in the steviol glycoside pathway takes up and modifies a steviol glycoside precursor in the cell; following modification in vivo, a steviol glycoside remains in the cell and/or is excreted into the culture medium. For example, a host cell expressing a gene encoding a polypeptide capable of synthesizing UTP from UDP, a gene encoding a polypeptide capable of converting glucose-6-phosphate to glucose-1-phosphate, a gene encoding a polypeptide capable of debranching glycogen, a gene encoding a polypeptide capable of synthesizing glucose-1-phosphate from phosphate and glycogen, and/or a gene encoding a polypeptide capable of synthesizing UDP-glucose from UTP and glucose-1-phosphate; and further expressing a gene encoding a polypeptide capable of glycosylating a steviol or a steviol glycoside at its C-13 hydroxyl group; a gene encoding a polypeptide capable of beta 1,3 glycosylation of the C3′ of the 13-O-glucose, 19-O-glucose, or both 13-O-glucose and 19-O-glucose of a steviol glycoside; a gene encoding a polypeptide capable of glycosylating the steviol or the steviol glycoside at its C-19 carboxyl group; and/or a gene encoding a polypeptide capable of beta 1,2 glycosylation of the C2′ of the 13-O-glucose, 19-O-glucose, or both 13-O-glucose and 19-O-glucose of a steviol glycoside can take up steviol and glycosylate steviol in the cell; following glycosylation in vivo, a steviol glycoside can be excreted into the culture medium. In certain such embodiments, the host cell may further express a gene encoding a polypeptide capable of synthesizing GGPP from FPP and IPP; a gene encoding a polypeptide capable of synthesizing ent-copalyl diphosphate from GGPP; a gene encoding a polypeptide capable of synthesizing ent-kaurene from ent-copalyl diphosphate; a gene encoding a polypeptide capable of synthesizing ent-kaurenoic acid, ent-kaurenol, and/or ent-kaurenal from ent-kaurene; a gene encoding a polypeptide capable of reducing cytochrome P450 complex; a gene encoding a polypeptide capable of synthesizing steviol from ent-kaurenoic acid; and/or a gene encoding a bifunctional polypeptide capable of synthesizing ent-copalyl diphosphate from GGPP and synthesizing ent-kaurene from ent-copalyl diphosphate.

In some embodiments, the method for producing one or more steviol glycosides or a steviol glycoside composition disclosed herein comprises whole-cell bioconversion of plant-derived or synthetic steviol and/or steviol glycosides in a cell culture medium of a recombinant host cell using: (a) a polypeptide capable of debranching glycogen, and/or (b) a polypeptide capable of synthesizing glucose-1-phosphate from phosphate and glycogen; optionally, one or more of: (c) a polypeptide capable of synthesizing UTP from UDP, (d) a polypeptide capable of converting glucose-6-phosphate to glucose-1-phosphate, and/or (e) a polypeptide capable of synthesizing UDP-glucose from UTP and glucose-1-phosphate; and one or more of: (f) a polypeptide capable of glycosylating the steviol or the steviol glycoside at its C-13 hydroxyl group thereof; (g) a polypeptide capable of beta 1,3 glycosylation of the C3′ of the 13-O-glucose, 19-O-glucose, or both 13-O-glucose and 19-O-glucose of a steviol glycoside; (h) a polypeptide capable of glycosylating the steviol or the steviol glycoside at its C-19 carboxyl group thereof; and/or (i) a polypeptide capable of beta 1,2 glycosylation of the C2′ of the 13-O-glucose, 19-O-glucose, or both 13-O-glucose and 19-O-glucose of a steviol glycoside; wherein at least one of the polypeptides is a recombinant polypeptide expressed in the recombinant host cell; and producing the one or more steviol glycosides or the steviol glycoside composition thereby.

In some embodiments of the methods for producing one or more steviol glycosides or a steviol glycoside composition disclosed herein comprises whole-cell bioconversion of plant-derived or synthetic steviol and/or steviol glycosides in a cell culture medium of a recombinant host cell disclosed herein, the polypeptide capable of debranching glycogen comprises a polypeptide having the amino acid sequence set forth in SEQ ID NO:157; and/or the polypeptide capable of synthesizing glucose-1-phosphate from phosphate and glycogen comprises a polypeptide having the amino acid sequence set forth in SEQ ID NO:159.

In some embodiments, a polypeptide capable of glycosylating a steviol or a steviol glycoside at its C-13 hydroxyl group thereof; a polypeptide capable of beta 1,3 glycosylation of the C3′ of the 13-O-glucose, 19-O-glucose, or both 13-O-glucose and 19-O-glucose of a steviol glycoside; a polypeptide capable of glycosylating the steviol or the steviol glycoside at its C-19 carboxyl group thereof; and/or a polypeptide capable of beta 1,2 glycosylation of the C2′ of the 13-O-glucose, 19-O-glucose, or both 13-O-glucose and 19-O-glucose of a steviol glycoside can be displayed on the surface of the recombinant host cells disclosed herein by fusing it with anchoring motifs.

In some embodiments, the cell is permeabilized to take up a substrate to be modified or to excrete a modified product. In some embodiments, a permeabilizing agent can be added to aid the feedstock entering into the host and product getting out. In some embodiments, the cells are permeabilized with a solvent such as toluene, or with a detergent such as Triton-X or Tween. In some embodiments, the cells are permeabilized with a surfactant, for example a cationic surfactant such as cetyltrimethylammonium bromide (CTAB). In some embodiments, the cells are permeabilized with periodic mechanical shock such as electroporation or a slight osmotic shock. For example, a crude lysate of the cultured microorganism can be centrifuged to obtain a supernatant. The resulting supernatant can then be applied to a chromatography column, e.g., a C18 column, and washed with water to remove hydrophilic compounds, followed by elution of the compound(s) of interest with a solvent such as methanol. The compound(s) can then be further purified by preparative HPLC. See also, WO 2009/140394.

In some embodiments, steviol, one or more steviol glycoside precursors, one or more steviol glycosides, or a steviol glycoside composition are produced by co-culturing of two or more hosts. In some embodiments, one or more hosts, each expressing one or more enzymes involved in the steviol glycoside pathway, produce steviol, one or more steviol glycoside precursors, and/or one or more steviol glycosides. For example, a host expressing a gene encoding a polypeptide capable of synthesizing GGPP from FPP and IPP; a gene encoding a polypeptide capable of synthesizing ent-copalyl diphosphate from GGPP; a gene encoding a polypeptide capable of synthesizing ent-kaurene from ent-copalyl diphosphate; a gene encoding a polypeptide capable of synthesizing ent-kaurenoic acid, ent-kaurenol, and/or ent-kaurenal from ent-kaurene; a gene encoding a polypeptide capable of reducing cytochrome P450 complex; a gene encoding a polypeptide capable of synthesizing steviol from ent-kaurenoic acid; and/or a gene encoding a bifunctional polypeptide capable of synthesizing ent-copalyl diphosphate from GGPP and synthesizing ent-kaurene from ent-copalyl diphosphate and a host expressing a gene encoding a polypeptide capable of synthesizing UTP from UDP, a gene encoding a polypeptide capable of converting glucose-6-phosphate to glucose-1-phosphate, a gene encoding a polypeptide capable of debranching glycogen, a gene encoding a polypeptide capable of synthesizing glucose-1-phosphate from phosphate and glycogen, and/or a gene encoding a polypeptide capable of synthesizing UDP-glucose from UTP and glucose-1-phosphate; and further expressing a gene encoding a polypeptide capable of glycosylating the steviol or the steviol glycoside at its C-13 hydroxyl group; a gene encoding a polypeptide capable of beta 1,3 glycosylation of the C3′ of the 13-O-glucose, 19-O-glucose, or both 13-O-glucose and 19-O-glucose of a steviol glycoside; a gene encoding a polypeptide capable of glycosylating the steviol or the steviol glycoside at its C-19 carboxyl group; and/or a gene encoding a polypeptide capable of beta 1,2 glycosylation of the C2′ of the 13-O-glucose, 19-O-glucose, or both 13-O-glucose and 19-O-glucose of a steviol glycoside, produce one or more steviol glycosides.

In some embodiments, the steviol glycoside comprises, for example, but not limited to, 13-SMG, steviol-1,2-bioside, steviol-1,3-bioside, 19-SMG, 1,2-stevioside, 1,3-stevioside (RebG), rubusoside, RebA, RebB, RebC, RebD, RebE, RebF, RebM, RebQ, RebI, dulcoside A, di-glycosylated steviol, tri-glycosylated steviol, tetra-glycosylated steviol, penta-glycosylated steviol, hexa-glycosylated steviol, hepta-glycosylated steviol, or isomers thereof.

In some embodiments, a steviol glycoside or steviol glycoside precursor composition produced in vivo, in vitro, or by whole cell bioconversion does not comprise or comprises a reduced amount or reduced level of plant-derived components than a Stevia extract from, inter alia, a Stevia plant. Plant-derived components can contribute to off-flavors and include pigments, lipids, proteins, phenolics, saccharides, spathulenol and other sesquiterpenes, labdane diterpenes, monoterpenes, decanoic acid, 8,11,14-eicosatrienoic acid, 2-methyloctadecane, pentacosane, octacosane, tetracosane, octadecanol, stigmasterol, β-sitosterol, α- and β-amyrin, lupeol, β-amryin acetate, pentacyclic triterpenes, centauredin, quercitin, epi-alpha-cadinol, carophyllenes and derivatives, beta-pinene, beta-sitosterol, and gibberellin. In some embodiments, the plant-derived components referred to herein are non-glycoside compounds.

As used herein, the terms “detectable amount,” “detectable concentration,” “measurable amount,” and “measurable concentration” refer to a level of steviol glycosides measured in AUC, μM/OD₆₀₀, mg/L, μM, or mM. Steviol glycoside production (i.e., total, supernatant, and/or intracellular steviol glycoside levels) can be detected and/or analyzed by techniques generally available to one skilled in the art, for example, but not limited to, liquid chromatography-mass spectrometry (LC-MS), thin layer chromatography (TLC), high-performance liquid chromatography (HPLC), ultraviolet-visible spectroscopy/spectrophotometry (UV-Vis), mass spectrometry (MS), and nuclear magnetic resonance spectroscopy (NMR).

As used herein, the term “undetectable concentration” refers to a level of a compound that is too low to be measured and/or analyzed by techniques such as TLC, HPLC, UV-Vis, MS, or NMR. In some embodiments, a compound of an “undetectable concentration” is not present in a steviol glycoside or steviol glycoside precursor composition.

After the recombinant microorganism has been grown in culture for the period of time, wherein the temperature and period of time facilitate the production of a steviol glycoside, steviol and/or one or more steviol glycosides can then be recovered from the culture using various techniques known in the art. Steviol glycosides can be isolated using a method described herein. For example, following fermentation, a culture broth can be centrifuged for 30 min at 7000 rpm at 4° C. to remove cells, or cells can be removed by filtration. The cell-free lysate can be obtained, for example, by mechanical disruption or enzymatic disruption of the host cells and additional centrifugation to remove cell debris. Mechanical disruption of the dried broth materials can also be performed, such as by sonication. The dissolved or suspended broth materials can be filtered using a micron or sub-micron prior to further purification, such as by preparative chromatography. The fermentation media or cell-free lysate can optionally be treated to remove low molecular weight compounds such as salt; and can optionally be dried prior to purification and re-dissolved in a mixture of water and solvent.

The supernatant or cell-free lysate can be purified as follows: a column can be filled with, for example, HP20 Diaion resin (aromatic type Synthetic Adsorbent; Supelco) or other suitable non-polar adsorbent or reversed-phase chromatography resin, and an aliquot of supernatant or cell-free lysate can be loaded on to the column and washed with water to remove the hydrophilic components. The steviol glycoside product can be eluted by stepwise incremental increases in the solvent concentration in water or a gradient from, e. g., 0%→100% methanol). The levels of steviol glycosides, glycosylated ent-kaurenol, and/or glycosylated ent-kaurenoic acid in each fraction, including the flow-through, can then be analyzed by LC-MS. Fractions can then be combined and reduced in volume using a vacuum evaporator. Additional purification steps can be utilized, if desired, such as additional chromatography steps and crystallization. For example, steviol glycosides can be isolated by methods not limited to ion exchange chromatography, reversed-phase chromatography (i.e., using a C18 column), extraction, crystallization, and carbon columns and/or decoloring steps.

In one embodiment, a recombinant host cell capable of producing one or more steviol glycosides or a steviol glycoside composition in a cell culture comprises a recombinant gene encoding a polypeptide capable of debranching glycogen; and/or a recombinant gene encoding a polypeptide capable of synthesizing glucose-1-phosphate from phosphate and glycogen, wherein the polypeptide capable of debranching glycogen is capable of 4-α-glucanotransferase activity and α-1,6-amyloglucosidase activity, wherein the recombinant host cell further comprises a gene encoding a polypeptide capable of synthesizing uridine 5′-triphosphate (UTP) from uridine diphosphate (UDP); a gene encoding a polypeptide capable of converting glucose-6-phosphate to glucose-1-phosphate; and/or a gene encoding a polypeptide capable of synthesizing uridine diphosphate glucose (UDP-glucose) from UTP and glucose-1-phosphate, wherein: the polypeptide capable of debranching glycogen comprises a polypeptide having at least 60% sequence identity to the amino acid sequence set forth in SEQ ID NO:157; the polypeptide capable of synthesizing glucose-1-phosphate from phosphate and glycogen comprises a polypeptide having at least 55% sequence identity to the amino acid sequence set forth in SEQ ID NO:159; the polypeptide capable of synthesizing UTP from UDP comprises a polypeptide having at least 60% sequence identity to the amino acid sequence set forth in SEQ ID NO:123; the polypeptide capable of converting glucose-6-phosphate to glucose-1-phosphate comprises a polypeptide having at least 60% sequence identity to the amino acid sequence set forth in any one of SEQ ID NO:2, 119, or 143 or a polypeptide having at least 55% sequence identity to the amino acid sequence set forth in any one of SEQ ID NOs:141, 145, or 147; and/or the polypeptide capable of synthesizing UDP-glucose from UTP and glucose-1-phosphate comprises a polypeptide having at least 60% sequence identity to the amino acid sequence set forth in any one of SEQ ID NO:121 or 127, a polypeptide having at least 55% sequence identity to the amino acid sequence set forth in any one of SEQ ID NOs:125, 129, 133, 135, 137, or 139 or a polypeptide having at least 70% sequence identity to the amino acid sequence set forth in SEQ ID NO:131.

In another embodiment, the recombinant host cell discussed above further comprises a gene encoding a polypeptide capable of glycosylating the steviol or the steviol glycoside at its C-13 hydroxyl group thereof; a gene encoding a polypeptide capable of beta 1,3 glycosylation of the C3′ of the 13-O-glucose, 19-O-glucose, or both 13-O-glucose and 19-O-glucose of the steviol glycoside; a gene encoding a polypeptide capable of glycosylating the steviol or the steviol glycoside at its C-19 carboxyl group thereof; and/or a gene encoding a polypeptide capable of beta 1,2 glycosylation of the C2′ of the 13-O-glucose, 19-O-glucose, or both 13-O-glucose and 19-O-glucose of the steviol glycoside; and further comprises a gene encoding a polypeptide capable of synthesizing geranylgeranyl pyrophosphate (GGPP) from farnesyl diphosphate (FPP) and isopentenyl diphosphate (IPP); a gene encoding a polypeptide capable of synthesizing ent-copalyl diphosphate from GGPP; a gene encoding an a polypeptide capable of synthesizing ent-kaurene from ent-copalyl diphosphate; a gene encoding a polypeptide capable of synthesizing ent-kaurenoic acid from ent-kaurene; a gene encoding a polypeptide capable of reducing cytochrome P450 complex; and/or a gene encoding a polypeptide capable of synthesizing steviol from ent-kaurenoic acid, wherein the polypeptide capable of glycosylating the steviol or the steviol glycoside at its C-13 hydroxyl group thereof comprises a polypeptide having at least 55% sequence identity to the amino acid sequence set forth in SEQ ID NO:7; the polypeptide capable of beta 1,3 glycosylation of the C3′ of the 13-O-glucose, 19-O-glucose, or both 13-O-glucose and 19-O-glucose of the steviol glycoside comprises a polypeptide having at least 50% sequence identity to the amino acid sequence set forth in SEQ ID NO:9; the polypeptide capable of glycosylating the steviol or the steviol glycoside at its C-19 carboxyl group thereof comprises a polypeptide having at least 55% sequence identity to the amino acid sequence set forth in SEQ ID NO:4; the polypeptide capable of beta 1,2 glycosylation of the C2′ of the 13-O-glucose, 19-O-glucose, or both 13-O-glucose and 19-O-glucose of a steviol glycoside comprises a polypeptide having at least 80% sequence identity to the amino acid sequence set forth in SEQ ID NO:11; a polypeptide having at least 80% sequence identity to the amino acid sequence set forth in SEQ ID NO:13; or a polypeptide having at least 65% sequence identity to the amino acid sequence set forth in SEQ ID NO:16; the polypeptide capable of synthesizing GGPP comprises a polypeptide having at least 70% sequence identity to the amino acid sequence set forth in any one of SEQ ID NO:20, 22, 24, 26, 28, 30, 32, or 116; the polypeptide capable of synthesizing ent-copalyl diphosphate comprises a polypeptide having at least 70% sequence identity to the amino acid sequence set forth in any one of SEQ ID NO:34, 36, 38, 40, 42, or 120; the polypeptide capable of synthesizing ent-kaurene comprises a polypeptide having at least 70% sequence identity to the amino acid sequence set forth in any one of SEQ ID NO:44, 46, 48, 50, or 52; the polypeptide capable of synthesizing ent-kaurenoic acid comprises a polypeptide having at least 70% sequence identity to the amino acid sequence set forth in any one of SEQ ID NOs:60, 62, 117, SEQ ID NO:66, 68, 70, 72, 74, or 76; the polypeptide capable of reducing cytochrome P450 complex comprises a polypeptide having at least 70% sequence identity to the amino acid sequence set forth in any one of SEQ ID NOs:78, 80, 82, 84, 86, 88, 90, 92; and/or the polypeptide capable of synthesizing steviol comprises a polypeptide having at least 70% sequence identity to the amino acid sequence set forth in any one of SEQ ID NOs:94, 97, 100, 101, 102, 103, 104, 106, 108, 110, 112, or 114.

In another embodiment, the recombinant host cell discussed above comprises a gene encoding a polypeptide capable of debranching glycogen having at least 60% sequence identity to the amino acid sequence set forth in SEQ ID NO:157; a gene encoding a polypeptide capable of synthesizing glucose-1-phosphate from phosphate and glycogen having at least 55% sequence identity to the amino acid sequence set forth in SEQ ID NO:159; a gene encoding a polypeptide capable of synthesizing uridine 5′-triphosphate (UTP) from uridine diphosphate (UDP) having at least 60% sequence identity to the amino acid sequence set forth in SEQ ID NO:123; a gene encoding a polypeptide capable of converting glucose-6-phosphate to glucose-1-phosphate having at least 60% sequence identity to the amino acid sequences set forth in any one of SEQ ID NOs:2 or 119; and a gene encoding a polypeptide capable of synthesizing UDP-glucose from UTP and glucose-1-phosphate having at least 60% sequence identity to the amino acid sequence set forth in SEQ ID NO:121; and one or more of: a gene encoding a polypeptide capable of glycosylating the steviol or the steviol glycoside at its C-13 hydroxyl group thereof having at least 55% sequence identity to the amino acid sequence set forth in SEQ ID NO:7; a gene encoding a polypeptide capable of beta 1,3 glycosylation of the C3′ of the 13-O-glucose, 19-O-glucose, or both 13-O-glucose and 19-O-glucose of a steviol glycoside having at least 50% sequence identity to the amino acid sequence set forth in SEQ ID NO:9; a gene encoding a polypeptide capable of glycosylating the steviol or the steviol glycoside at its C-19 carboxyl group thereof having at least 55% sequence identity to the amino acid sequence set forth in SEQ ID NO:4; a gene encoding a polypeptide capable of beta 1,2 glycosylation of the C2′ of the 13-O-glucose, 19-O-glucose, or both 13-O-glucose and 19-O-glucose of a steviol glycoside comprises a polypeptide having at least 80% sequence identity to the amino acid sequence set forth in SEQ ID NO:11; a polypeptide having at least 80% sequence identity to the amino acid sequence set forth in SEQ ID NO:13; or a polypeptide having at least 65% sequence identity to the amino acid sequence set forth in SEQ ID NO:16.

In another embodiment, the recombinant host cell discussed above comprises a gene encoding a polypeptide capable of debranching glycogen having at least 60% sequence identity to the amino acid sequence set forth in SEQ ID NO:157; and/or a gene encoding a polypeptide capable of synthesizing glucose-1-phosphate from phosphate and glycogen having at least 55% sequence identity to the amino acid sequence set forth in SEQ ID NO:159; wherein the gene encoding a polypeptide capable of debranching glycogen and/or the gene encoding a polypeptide capable of synthesizing glucose-1-phosphate from phosphate and glycogen are overexpressed relative to a corresponding host cell lacking the one or more recombinant genes, wherein the gene encoding a polypeptide capable of debranching glycogen and/or the gene encoding a polypeptide capable of synthesizing glucose-1-phosphate from phosphate and glycogen are overexpressed by at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% relative to a corresponding host cell lacking the one or more recombinant genes.

In another embodiment, the expression of the one or more recombinant genes comprising the recombinant host cell increase the amount of UDP-glucose accumulated by the recombinant host cell relative to a corresponding host lacking the one or more recombinant genes, wherein expression of the one or more recombinant genes increases the amount of UDP-glucose accumulated by the cell by at least 10%, at least 25%, or at least 50%, at least 100%, at least 150%, at least 200%, or at least 250% relative to a corresponding host lacking the one or more recombinant genes, wherein expression of the one or more recombinant genes increases an amount of the one or more steviol glycosides or the steviol glycoside composition produced by the cell relative to a corresponding host lacking the one or more recombinant genes, wherein expression of the one or more recombinant genes increases the amount of the one or more steviol glycosides produced by the cell by at least 5%, or at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% relative to a corresponding host cell lacking the one or more recombinant genes, wherein expression of the one or more recombinant genes increases an amount of RebA, RebD, and/or RebM produced by the cell by at least 5%, or at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% relative to a corresponding host cell lacking the one or more recombinant genes, wherein expression of the one or more recombinant genes decreases the amount of the one of one or more steviol glycosides or the steviol glycoside composition accumulated by the cell relative to a corresponding host lacking the one or more recombinant genes, wherein expression of the one or more recombinant genes decreases the amount of the one or more steviol glycosides accumulated by the cell by at least 5%, or at least 10%, or at least 15%, or at least 20%, or at least 30%, or at least 40%, or at least 50% relative to a corresponding host cell lacking the one or more recombinant genes relative to a corresponding host lacking the one or more recombinant genes, wherein expression of the one or more recombinant genes decreases an amount of 13-SMG accumulated by the cell relative to a corresponding host lacking the one or more recombinant genes, wherein expression of the one or more recombinant genes increases the amount of total steviol glycosides produced by the cell by at least 5%, or at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% relative to a corresponding host lacking the one or more recombinant genes, and/or wherein expression of the one or more recombinant genes decreases the amount of total steviol glycosides produced by the cell by less than 10%, or less than 5%, or less than 2.5% relative to a corresponding host lacking the one or more recombinant genes.

In one embodiment of the recombinant host cells discussed above, the one or more steviol glycosides is, or the steviol glycoside composition comprises, steviol-13-O-glucoside (13-SMG), steviol-1,2-Bioside, steviol-1,3-Bioside, steviol-19-O-glucoside (19-SMG), 1,2-Stevioside, 1,3-stevioside (RebG), rubusoside, rebaudioside A (RebA), rebaudioside B (RebB), rebaudioside C (RebC), rebaudioside D (RebD), rebaudioside E (RebE), rebaudioside F (RebF), rebaudioside M (RebM), rebaudioside Q (RebQ), rebaudioside I (RebI), dulcoside A, and/or an isomer thereof.

In one embodiment of the recombinant host cells discussed above, the recombinant host cell is a plant cell, a mammalian cell, an insect cell, a fungal cell, an algal cell or a bacterial cell.

In one embodiment, a method of producing one or more steviol glycosides or a steviol glycoside composition in a cell culture, comprises culturing the recombinant host cells discussed above in the cell culture, under conditions in which the genes are expressed, and wherein the one or more steviol glycosides or the steviol glycoside composition is produced by the recombinant host cells, wherein the genes are constitutively expressed or wherein the expression of the genes is induced, wherein the amount of RebA, RebD, and/or RebM produced by the cell is increased by at least 5%, or at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% relative to a corresponding host lacking the one or more recombinant genes, wherein the amount of 13-SMG accumulated by the cell is decreased by at least 10%, at least 25%, or at least 50% relative to a corresponding host lacking the one or more recombinant genes, wherein the amount of total steviol glycosides produced by the cell is increased by at least 5%, or at least 10%, or at least 15%, or at least 20%, or at least 30%, or at least 40%, or at least 50%, or at least 60%, or at least 70%, or at least 80%, or at least 90%, or at least 100%, or at least 125%, or at least 150%, or at least 175%, or at least 200% relative to a corresponding host lacking the one or more recombinant genes, wherein the amount of total steviol glycosides produced by the cell decreases the amount of total steviol glycosides produced by the cell by less than 10%, or less than 5%, or less than 2.5% relative to a corresponding host lacking the one or more recombinant genes, wherein the recombinant host cell is grown in a fermentor at a temperature for a period of time, wherein the temperature and period of time facilitate the production of the one or more steviol glycosides or the steviol glycoside composition, and/or wherein the amount of UDP-glucose accumulated by the cell by at least 10%, at least 25%, or at least 50%, at least 100%, at least 150%, at least 200%, or at least 250% relative to a corresponding host lacking the one or more recombinant genes.

In one embodiment, the method of producing one or more steviol glycosides or a steviol glycoside composition in a cell culture further comprises isolating the produced one or more steviol glycosides or the steviol glycoside composition from the cell culture, wherein the isolating step comprises separating a liquid phase of the cell culture from a solid phase of the cell culture to obtain a supernatant comprising the produced one or more steviol glycosides or the steviol glycoside composition, and contacting the supernatant with one or more adsorbent resins in order to obtain at least a portion of the produced one or more steviol glycosides or the steviol glycoside composition; or contacting the supernatant with one or more ion exchange or reversed-phase chromatography columns in order to obtain at least a portion of the produced one or more steviol glycosides or the steviol glycoside composition; or crystallizing or extracting the produced one or more steviol glycosides or the steviol glycoside composition; thereby isolating the produced one or more steviol glycosides or the steviol glycoside composition.

In one embodiment, the method of producing one or more steviol glycosides or a steviol glycoside composition in a cell culture further comprises recovering the one or more steviol glycosides or the steviol glycoside composition from the cell culture, wherein the produced one or more steviol glycosides or the steviol glycoside composition is enriched for the one or more steviol glycosides relative to a steviol glycoside composition of Stevia plant and has a reduced level of Stevia plant-derived components relative to a steviol glycoside composition obtained from a plant-derived Stevia extract.

In one embodiment, the method of producing one or more steviol glycosides or a steviol glycoside composition comprises whole-cell bioconversion of a plant-derived or synthetic steviol and/or steviol glycosides in a cell culture of a recombinant host cell using a polypeptide capable of debranching glycogen, comprising a polypeptide having at least 60% sequence identity to the amino acid sequence set forth in SEQ ID NO:157; and/or a polypeptide capable of synthesizing glucose-1-phosphate from phosphate and glycogen, comprising a polypeptide having at least 55% sequence identity to the amino acid sequence set forth in SEQ ID NO:159; and optionally, one or more of a polypeptide capable of synthesizing UTP from UDP, comprising a polypeptide having at least 60% sequence identity to the amino acid sequence set forth in SEQ ID NO:123; a polypeptide capable of converting glucose-6-phosphate to glucose-1-phosphate, comprising a polypeptide having at least 60% sequence identity to the amino acid sequence set forth in any one of SEQ ID NO:2, 119, or 143; or at least 55% sequence identity to the amino acid sequence set forth in any one of SEQ ID NO:141, 145, or 147; and/or a polypeptide capable of synthesizing UDP-glucose from UTP and glucose-1-phosphate, comprising a polypeptide having at least 60% sequence identity to the amino acid sequence set forth in any one of SEQ ID NO:121 or 127; at least 55% sequence identity to the amino acid sequence set forth in any one of SEQ ID NOs:125, 129, 133, 135, 137, or 139; or at least 70% sequence identity to the amino acid sequence set forth in SEQ ID NO:131, and one or more of a polypeptide capable of glycosylating a steviol or the steviol glycoside at its C-13 hydroxyl group thereof; a polypeptide capable of beta 1,3 glycosylation of the C3′ of the 13-O-glucose, 19-O-glucose, or both 13-O-glucose and 19-O-glucose of a steviol glycoside; a polypeptide capable of glycosylating the steviol or the steviol glycoside at its C-19 carboxyl group thereof; and/or a polypeptide capable of beta 1,2 glycosylation of the C2′ of the 13-O-glucose, 19-O-glucose, or both 13-O-glucose and 19-O-glucose of a steviol glycoside; wherein at least one of the polypeptides is a recombinant polypeptide expressed in the recombinant host cell; and producing the one or more steviol glycosides or the steviol glycoside composition thereby, wherein the polypeptide capable of glycosylating the steviol or the steviol glycoside at its C-13 hydroxyl group thereof comprises a polypeptide having at least 55% sequence identity to the amino acid sequence set forth in SEQ ID NO:7; the polypeptide capable of beta 1,3 glycosylation of the C3′ of the 13-O-glucose, 19-O-glucose, or both 13-O-glucose and 19-O-glucose of the steviol glycoside comprises a polypeptide having at least 50% sequence identity to the amino acid sequence set forth in SEQ ID NO:9; the polypeptide capable of glycosylating the steviol or the steviol glycoside at its C-19 carboxyl group thereof comprises a polypeptide having at least 55% sequence identity to the amino acid sequence set forth in SEQ ID NO:4; the polypeptide capable of beta 1,2 glycosylation of the C2′ of the 13-O-glucose, 19-O-glucose, or both 13-O-glucose and 19-O-glucose of the steviol glycoside comprises a polypeptide having at least 80% sequence identity to the amino acid sequence set forth in SEQ ID NO:11; a polypeptide having at least 80% sequence identity to the amino acid sequence set forth in SEQ ID NO:13; or a polypeptide having at least 65% sequence identity to the amino acid sequence set forth in SEQ ID NO:16.

In one embodiment, the recombinant host cell used in the method of producing one or more steviol glycosides or a steviol glycoside composition in a cell culture is a plant cell, a mammalian cell, an insect cell, a fungal cell, an algal cell, or a bacterial cell, wherein the one or more steviol glycosides is, or the steviol glycoside composition comprises, steviol-13-O-glucoside (13-SMG), steviol-1,2-Bioside, steviol-1,3-Bioside, steviol-19-O-glucoside (19-SMG), 1,2-stevioside, 1,3-stevioside (RebG), rubusoside, rebaudioside A (RebA), rebaudioside B (RebB), rebaudioside C (RebC), rebaudioside D (RebD), rebaudioside E (RebE), rebaudioside F (RebF), rebaudioside M (RebM), rebaudioside Q (RebQ), rebaudioside I (RebI), dulcoside A, and/or an isomer thereof.

As used herein, the terms “or” and “and/or” is utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” In some embodiments, “and/or” is used to refer to the exogenous nucleic acids that a recombinant cell comprises, wherein a recombinant cell comprises one or more exogenous nucleic acids selected from a group. In some embodiments, “and/or” is used to refer to production of steviol glycosides and/or steviol glycoside precursors. In some embodiments, “and/or” is used to refer to production of steviol glycosides, wherein one or more steviol glycosides are produced. In some embodiments, “and/or” is used to refer to production of steviol glycosides, wherein one or more steviol glycosides are produced through one or more of the following steps: culturing a recombinant microorganism, synthesizing one or more steviol glycosides in a recombinant microorganism, and/or isolating one or more steviol glycosides.

### Functional Homologs

Functional homologs of the polypeptides described above are also suitable for use in producing steviol glycosides in a recombinant host. A functional homolog is a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of steviol glycoside biosynthesis polypeptides. Sequence analysis can involve BLAST, Reciprocal BLAST, or PSI-BLAST analysis of non-redundant databases using a UGT amino acid sequence as the reference sequence. Amino acid sequence is, in some instances, deduced from the nucleotide sequence. Those polypeptides in the database that have greater than 40% sequence identity are candidates for further evaluation for suitability as a steviol glycoside biosynthesis polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in steviol glycoside biosynthesis polypeptides, e.g., conserved functional domains. In some embodiments, nucleic acids and polypeptides are identified from transcriptome data based on expression levels rather than by using BLAST analysis.

Conserved regions can be identified by locating a region within the primary amino acid sequence of a steviol glycoside biosynthesis polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. The information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some embodiments, alignment of sequences from two different species is adequate to identify such homologs.

Typically, polypeptides that exhibit at least 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

For example, polypeptides suitable for producing steviol in a recombinant host include functional homologs of UGTs.

Methods to modify the substrate specificity of, for example, a UGT, are known to those skilled in the art, and include without limitation site-directed/rational mutagenesis approaches, random directed evolution approaches and combinations in which random mutagenesis/saturation techniques are performed near the active site of the enzyme. For example see Osmani et al., 2009, Phytochemistry 70: 325-347.

A candidate sequence typically has a length that is from 80% to 200% of the length of the reference sequence, e.g., 82, 85, 87, 89, 90, 93, 95, 97, 99, 100, 105, 110, 115, 120, 130, 140, 150, 160, 170, 180, 190, or 200% of the length of the reference sequence. A functional homolog polypeptide typically has a length that is from 95% to 105% of the length of the reference sequence, e.g., 90, 93, 95, 97, 99, 100, 105, 110, 115, or 120% of the length of the reference sequence, or any range between. A % identity for any candidate nucleic acid or polypeptide relative to a reference nucleic acid or polypeptide can be determined as follows. A reference sequence (e.g., a nucleic acid sequence or an amino acid sequence described herein) is aligned to one or more candidate sequences using the computer program Clustal Omega (version 1.2.1, default parameters), which allows alignments of nucleic acid or polypeptide sequences to be carried out across their entire length (global alignment). Chenna et al., 2003, Nucleic Acids Res. 31(13):3497-500.

Clustal Omega calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: %age; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: % age; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The Clustal Omega output is a sequence alignment that reflects the relationship between sequences. Clustal Omega can be run, for example, at the Baylor College of Medicine Search Launcher site on the World Wide Web (searchlauncher.bcm.tmc.edu/multi-align/multi-align.html) and at the European Bioinformatics Institute site at http://www.ebi.ac.uk/Tools/msa/clustalo/.

To determine a % identity of a candidate nucleic acid or amino acid sequence to a reference sequence, the sequences are aligned using Clustal Omega, the number of identical matches in the alignment is divided by the length of the reference sequence, and the result is multiplied by 100. It is noted that the % identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

It will be appreciated that functional UGT proteins (e.g., a polypeptide capable of glycosylating a steviol or a steviol glycoside at its C-19 carboxyl group) can include additional amino acids that are not involved in the enzymatic activities carried out by the enzymes. In some embodiments, UGT proteins are fusion proteins. The terms “chimera,” “fusion polypeptide,” “fusion protein,” “fusion enzyme,” “fusion construct,” “chimeric protein,” “chimeric polypeptide,” “chimeric construct,” and “chimeric enzyme” can be used interchangeably herein to refer to proteins engineered through the joining of two or more genes that code for different proteins.

In some embodiments, a nucleic acid sequence encoding a UGT polypeptide (e.g., a polypeptide capable of glycosylating the steviol or the steviol glycoside at its C-19 carboxyl group) can include a tag sequence that encodes a “tag” designed to facilitate subsequent manipulation (e.g., to facilitate purification or detection), secretion, or localization of the encoded polypeptide. Tag sequences can be inserted in the nucleic acid sequence encoding the polypeptide such that the encoded tag is located at either the carboxyl or amino terminus of the polypeptide. Non-limiting examples of encoded tags include green fluorescent protein (GFP), human influenza hemagglutinin (HA), glutathione S transferase (GST), polyhistidine-tag (HIS tag), and Flag™ tag (Kodak, New Haven, Conn.). Other examples of tags include a chloroplast transit peptide, a mitochondrial transit peptide, an amyloplast peptide, signal peptide, or a secretion tag.

In some embodiments, a fusion protein is a protein altered by domain swapping. As used herein, the term “domain swapping” is used to describe the process of replacing a domain of a first protein with a domain of a second protein. In some embodiments, the domain of the first protein and the domain of the second protein are functionally identical or functionally similar. In some embodiments, the structure and/or sequence of the domain of the second protein differs from the structure and/or sequence of the domain of the first protein. In some embodiments, a UGT polypeptide (e.g., a polypeptide capable of glycosylating a steviol or a steviol glycoside at its C-19 carboxyl group) is altered by domain swapping.

In some embodiments, a fusion protein is a protein altered by circular permutation, which consists in the covalent attachment of the ends of a protein that would be opened elsewhere afterwards. Thus, the order of the sequence is altered without causing changes in the amino acids of the protein. In some embodiments, a targeted circular permutation can be produced, for example but not limited to, by designing a spacer to join the ends of the original protein. Once the spacer has been defined, there are several possibilities to generate permutations through generally accepted molecular biology techniques, for example but not limited to, by producing concatemers by means of PCR and subsequent amplification of specific permutations inside the concatemer or by amplifying discrete fragments of the protein to exchange to join them in a different order. The step of generating permutations can be followed by creating a circular gene by binding the fragment ends and cutting back at random, thus forming collections of permutations from a unique construct.

### Steviol and Steviol Glycoside Biosynthesis Nucleic Acids

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In many cases, the coding sequence for a polypeptide described herein is identified in a species other than the recombinant host, i.e., is a heterologous nucleic acid. Thus, if the recombinant host is a microorganism, the coding sequence can be from other prokaryotic or eukaryotic microorganisms, from plants or from animals. In some case, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. “Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements.

One or more genes can be combined in a recombinant nucleic acid construct in “modules” useful for a discrete aspect of steviol and/or steviol glycoside production. Combining a plurality of genes in a module, particularly a polycistronic module, facilitates the use of the module in a variety of species. For example, a steviol biosynthesis gene cluster, or a UGT gene cluster, can be combined in a polycistronic module such that, after insertion of a suitable regulatory region, the module can be introduced into a wide variety of species. As another example, a UGT gene cluster can be combined such that each UGT coding sequence is operably linked to a separate regulatory region, to form a UGT module. Such a module can be used in those species for which monocistronic expression is necessary or desirable. In addition to genes useful for a steviol or steviol glycoside production, a recombinant construct typically also contains an origin of replication, and one or more selectable markers for maintenance of the construct in appropriate species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host is obtained, using appropriate codon bias tables for that host (e.g., microorganism). As isolated nucleic acids, these modified sequences can exist as purified molecules and can be incorporated into a vector or a virus for use in constructing modules for recombinant nucleic acid constructs.

In some cases, it is desirable to inhibit one or more functions of an endogenous polypeptide in order to divert metabolic intermediates towards a steviol or steviol glycoside biosynthesis. For example, it may be desirable to downregulate synthesis of sterols in a yeast strain in order to further increase the steviol or the steviol glycoside production, e.g., by downregulating squalene epoxidase. As another example, it may be desirable to inhibit degradative functions of certain endogenous gene products, e.g., glycohydrolases that remove glucose moieties from secondary metabolites or phosphatases as discussed herein. In such cases, a nucleic acid that overexpresses the polypeptide or gene product may be included in a recombinant construct that is transformed into the strain. Alternatively, mutagenesis can be used to generate mutants in genes for which it is desired to increase or enhance function.

### Host Microorganisms

Recombinant hosts can be used to express polypeptides for producing steviol glycosides, including, but not limited to, a plant cell, comprising a plant cell that is grown in a plant, a mammalian cell, an insect cell, a fungal cell, an algal cell, or a bacterial cell.

A number of prokaryotes and eukaryotes are also suitable for use in constructing the recombinant microorganisms described herein, e.g., gram-negative bacteria, yeast, and fungi. A species and strain selected for use as a steviol glycoside production strain is first analyzed to determine which production genes are endogenous to the strain and which genes are not present. Genes for which an endogenous counterpart is not present in the strain are advantageously assembled in one or more recombinant constructs, which are then transformed into the strain in order to supply the missing function(s).

Typically, the recombinant microorganism is grown in a fermenter at a temperature(s) for a period of time, wherein the temperature and period of time facilitate the production of a steviol glycoside. The constructed and genetically engineered microorganisms provided by the invention can be cultivated using conventional fermentation processes, including, inter alia, chemostat, batch, fed-batch cultivations, semi-continuous fermentations such as draw and fill, continuous perfusion fermentation, and continuous perfusion cell culture. Depending on the particular microorganism used in the method, other recombinant genes such as isopentenyl biosynthesis genes and terpene synthase and cyclase genes may also be present and expressed. Levels of substrates and intermediates, e.g., isopentenyl diphosphate, dimethylallyl diphosphate, GGPP, ent-kaurene and ent-kaurenoic acid, can be determined by extracting samples from culture media for analysis according to published methods.

In some aspects, the recombinant microorganism is grown in a deep well plate. It will be understood that while data on production of steviol glycosides by the recombinant microorganism grown in deep well cultures, in some aspects, may be more easily collected than that in fermentation cultures, the small culture volume of the deep well (e.g., 1 ml or 0.5 ml) can effect differences in the environment of the microorganism and, therefore its efficiency and effectiveness in producing steviol glycosides. For example, nutrient availability, cellular waste product buildup, pH, temperature, agitation, and aeration may differ significantly between fermentation and deep well cultures. Accordingly, uptake of nutrients or other enzyme substrates may vary, affecting the cellular metabolism (e.g., changing the amount and/or profile of products accumulated by a recombinant microorganism). See, e.g., Duetz, Trends Microbiol 15(10):469-75 (2007).

Carbon sources of use in the instant method include any molecule that can be metabolized by the recombinant host cell to facilitate growth and/or production of the steviol glycosides. Examples of suitable carbon sources include, but are not limited to, sucrose (e.g., as found in molasses), fructose, xylose, ethanol, glycerol, glucose, cellulose, starch, cellobiose or other glucose-comprising polymer. In embodiments employing yeast as a host, for example, carbons sources such as sucrose, fructose, xylose, ethanol, glycerol, and glucose are suitable. The carbon source can be provided to the host organism throughout the cultivation period or alternatively, the organism can be grown for a period of time in the presence of another energy source, e.g., protein, and then provided with a source of carbon only during the fed-batch phase.

It will be appreciated that the various genes and modules discussed herein can be present in two or more recombinant hosts rather than a single host. When a plurality of recombinant hosts is used, they can be grown in a mixed culture to accumulate steviol and/or steviol glycosides.

Alternatively, the two or more hosts each can be grown in a separate culture medium and the product of the first culture medium, e.g., steviol, can be introduced into second culture medium to be converted into a subsequent intermediate, or into an end product such as, for example, RebA. The product produced by the second, or final host is then recovered. It will also be appreciated that in some embodiments, a recombinant host is grown using nutrient sources other than a culture medium and utilizing a system other than a fermenter.

Exemplary prokaryotic and eukaryotic species are described in more detail below. However, it will be appreciated that other species can be suitable. However, it will be appreciated that other species can be suitable to express polypeptides for the producing steviol glycosides.

For example, suitable species can be in a genus such as Agaricus, Aspergillus, Bacillus, Candida, Corynebacterium, Eremothecium, Escherichia, Fusarium/Gibberella, Kluyveromyces, Laetiporus, Lentinus, Phaffia, Phanerochaete, Pichia (formally known as Hansuela), Scheffersomyces, Physcomitrella, Rhodoturula, Saccharomyces, Schizosaccharomyces, Sphaceloma, Xanthophyllomyces, Humicola, lssatchenkia, Brettanomyces, Yamadazyma, Lachancea, Zygosaccharomyces, Komagataella, Kazachstania, Xanthophyllomyces, Geotrichum, Blakeslea, Dunaliella, Haematococcus, Chlorella, Undaria, Sargassum, Laminaria, Scenedesmus, Pachysolen, Trichosporon, Acremonium, Aureobasidium, Cryptococcus, Corynascus, Chrysosporium, Filibasidium, Fusarium, Magnaporthe, Monascus, Mucor, Myceliophthora, Mortierella, Neocallimastix, Neurospora, Paecilomyces, Penicillium, Piromyces, Pachysolen, Phanerochaete, Podospora, Pycnoporus, Rhizopus, Schizophyllum, Sordaria, Talaromyces, Rasmsonia, Thermoascus, Thielavia, Tolypocladium, Kloeckera, Pachysolen, Schwanniomyces, Trametes, Trichoderma, Acinetobacter, Nocardia, Xanthobacter, Streptomyces, Erwinia, Klebsiella, Serratia, Pseudomonas, Salmonella, Choroflexus, Chloronema, Chlorobium, Pelodictyon, Chromatium, Rhode-spirillum, Rhodobacter, Rhodomicrobium, or Yarrowia.

Exemplary species from such genera include Lentinus tigrinus, Laetiporus sulphureus, Phanerochaete chrysosporium, Pichia pastoris, Pichia kudriavzevii, Cyberlindnera jadinii, Physcomitrella patens, Rhodoturula glutinis, Rhodoturula mucilaginosa, Phaffia rhodozyma, Xanthophyllomyces dendrorhous, lssatchenkia orientalis, Saccharomyces cerevisiae, Saccharomyces bayanus, Saccharomyces pastorianus, Saccharomyces carlsbergensis, Hansuela polymorpha, Brettanomyces anomalus, Yamadazyma philogaea, Fusarium fujikuroil Gibberella fujikuroi, Candida utilis, Candida glabrata, Candida krusei, Candida revkaufi, Candida pulcherrima, Candida tropicalis, Aspergillus niger, Aspergillus oryzae, Aspergillus fumigatus, Penicillium chrysogenum, Penicillium citrinum, Acremonium chrysogenum, Trichoderma reesei, Rasamsonia emersonfi (formerly known as Talaromyces emersonfi), Aspergillus sojae, Chrysosporium lucknowense, Myceliophtora thermophyla, Candida albicans, Bacillus subtilis, Bacillus amyloliquefaciens, Bacillius licheniformis, Bacillus puntis, Bacillius megaterium, Bacillius halofurans, Baciilius punilus, Serratia marcessans, Pseudomonas aeruginosa, Salmonella typhimurium, Blakeslea trispora, Dunaliella salina, Haematococcus pluvialis, Chlorella sp., Undaria pinnatifida, Sargassum, Laminaria japonica, Scenedesmus almeriensis, Salmonella typhi, Choroflexus aurantiacus, Chloronema gigateum, Chlorobium limicola, Pelodictyon luteolum, Chromatium okenii, Rhode-spirillum rubrum, Rhodobacter spaeroides, Rhodobacter capsulatus, Rhodomicrobium vanellii, Pachysolen tannophilus, Trichosporon beigelii, and Yarrowia lipolytica.

In some embodiments, a microorganism can be a prokaryote such as Escherichia bacteria cells, for example, Escherichia coli cells; Lactobacillus bacteria cells; Lactococcus bacteria cells; Cornebacterium bacteria cells; Acetobacter bacteria cells; Acinetobacter bacteria cells; or Pseudomonas bacterial cells.

In some embodiments, a microorganism can be an algal cell such as Blakeslea trispora, Dunaliella salina, Haematococcus pluvialis, Chlorella sp., Undaria pinnatifida, Sargassum, Laminaria japonica, Scenedesmus almeriensis species.

In some embodiments, a microorganism can be a fungi from the genera including but not limited to Acremonium, Arxula, Agaricus, Aspergillus, Agaricus, Aureobasidium, Brettanomyces, Candida, Cryptococcus, Corynascus, Chrysosporium, Debaromyces, Filibasidium, Fusarium, Gibberella, Humicola, Magnaporthe, Monascus, Mucor, Myceliophthora, Mortierella, Neocallimastix, Neurospora, Paecilomyces, Penicillium, Piromyces, Phanerochaete Podospora, Pycnoporus, Rhizopus, Schizophyllum, Schizosaccharomyces, Sordaria, Scheffersomyces, Talaromyces, Rhodotorula, Rhodosporidium, Rasmsonia, Zygosaccharomyces, Thermoascus, Thielavia, Trichosporon, Tolypocladium, Trametes, and Trichoderma. Fungal species include, but are not limited to, Aspergillus niger, Aspergillus oryzae, Aspergillus fumigatus, Penicillium chrysogenum, Penicillium citrinum, Acremonium chrysogenum, Trichoderma reesei, Rasamsonia emersonii (formerly known as Talaromyces emersonii), Aspergillus sojae, Chrysosporium lucknowense, Myceliophtora thermophyla.

In some embodiments, a microorganism can be an Ascomycete such as Gibberella fujikuroi, Kluyveromyces lactis, Schizosaccharomyces pombe, Geotrichum Aspergillus niger, Yarrowia lipolytica, Ashbya gossypii, Yamadazyma philogaea, Lachancea kluyveri, Kodamaea ohmeri, or S. cerevisiae.

### Agaricus, Gibberella, and Phanerochaete spp.

Agaricus, Gibberella, and Phanerochaete spp. can be useful because they are known to produce large amounts of isoprenoids in culture. Thus, the terpene precursors for producing large amounts of steviol glycosides are already produced by endogenous genes. Thus, modules comprising recombinant genes for steviol glycoside biosynthesis polypeptides can be introduced into species from such genera without the necessity of introducing mevalonate or MEP pathway genes.

### Arxula Adeninivorans (Blastobotrys Adeninivorans)

Arxula adeninivorans is dimorphic yeast (it grows as budding yeast like the baker's yeast up to a temperature of 42° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

### Rhodotorula sp.

Rhodotorula is unicellular, pigmented yeast. The oleaginous red yeast, Rhodotorula glutinis, has been shown to produce lipids and carotenoids from crude glycerol (Saenge et al., 2011, Process Biochemistry 46(1):210-8). Rhodotorula toruloides strains have been shown to be an efficient fed-batch fermentation system for improved biomass and lipid productivity (Li et al., 2007, Enzyme and Microbial Technology 41:312-7).

### Schizosaccharomyces spp.

Schizosaccharomyces is a genus of fission yeasts. Similar to S. cerevisiae, Schizosaccharomyces is a model organism in the study of eukaryotic cell biology. It provides an evolutionary distant comparison to S. cerevisiae. Species include but are not limited to S. cryophilius and S. pombe. (See Hoffman et al., 2015, Genetics. 201(2):403-23).

### Humicola spp.

Humicola is a genus of filamentous fungi. Species include but are not limited to H. alopallonella and H. siamensis.

### Brettanomyces spp.

Brettanomyces is a non-spore forming genus of yeast. It is from the Saccharomycetaceae family and commonly used in the brewing and wine industries. Brettanomyces produces several sensory compounds that contribute to the complexity of wine, specifically red wine. Brettanomyces species include but are not limited to B. bruxellensis and B. claussenii. See, e.g., Fugelsang et al., 1997, Wine Microbiology.

### Trichosporon spp.

Trichosporon is a genus of the fungi family. Trichosporon species are yeast commonly isolated from the soil, but can also be found in the skin microbiota of humans and animals. Species include, for example but are not limited to, T. aquatile, T. beigelii, and T. dermatis.

### Debaromyces spp.

Debaromyces is a genus of the ascomycetous yeast family, in which species are characterized as a salt-tolerant marine species. Species include but are not limited to D. hansenii and D. hansenius.

Physcomitrella spp.

Physcomitrella mosses, when grown in suspension culture, have characteristics similar to yeast or other fungal cultures. This genera can be used for producing plant secondary metabolites, which can be difficult to produce in other types of cells.

Saccharomyces spp.

Saccharomyces is a widely used chassis organism in synthetic biology, and can be used as the recombinant microorganism platform. For example, there are libraries of mutants, plasmids, detailed computer models of metabolism and other information available for S. cerevisiae, allowing for rational design of various modules to enhance product yield. Methods are known for making recombinant microorganisms. Examples of Saccharomyces species include S. castellii, also known as Naumovozyma castelli.

Zygosaccharomyces spp.

Zygosaccharomyces is a genus of yeast. Originally classified under the Saccharomyces genus it has since been reclassified. It is widely known in the food industry because several species are extremely resistant to commercially used food preservation techniques. Species include but are not limited to Z. bisporus and Z. cidri. (See Barnett et al, Yeasts: Characteristics and Identification, 1983).

### Geotrichum spp.

Geotrichum is a fungi commonly found in soil, water and sewage worldwide. It's often identified in plants, cereal and dairy products. Species include, for example but are not limited to, G. candidum and G. klebahnii (see Carmichael et al., Mycologica, 1957, 49(6):820-830.)

### Kazachstania sp

Kazachstania is a yeast genus in the family Sacchromycetaceae.

### Torulaspora spp.

Torulaspora is a genus of yeasts and species include but are not limited to T. franciscae and T. globosa.

### Aspergillus spp.

Aspergillus species such as A. oryzae, A. niger and A. sojae are widely used microorganisms in food production and can also be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for producing steviol glycosides.

### Yarrowia Lipolytica

Yarrowia lipolytica is dimorphic yeast (see Arxula adeninivorans) and belongs to the family Hemiascomycetes. The entire genome of Yarrowia lipolytica is known. Yarrowia species is aerobic and considered to be non-pathogenic. Yarrowia is efficient in using hydrophobic substrates (e.g., alkanes, fatty acids, and oils) and can grow on sugars. It has a high potential for industrial applications and is an oleaginous microorganism. Yarrowia lipolyptica can accumulate lipid content to approximately 40% of its dry cell weight and is a model organism for lipid accumulation and remobilization. See e.g., Nicaud, 2012, Yeast 29(10):409-18; Beopoulos et al., 2009, Biochimie 91(6):692-6; Bankar et al., 2009, Appl Microbiol Biotechnol. 84(5):847-65.

### Rhodosporidium Toruloides

Rhodosporidium toruloides is oleaginous yeast and useful for engineering lipid-production pathways (See e.g. Zhu et al., 2013, Nature Commun. 3:1112; Ageitos et al., 2011, Applied Microbiology and Biotechnology 90(4):1219-27).

### Candida Boidinii

Candida boidinii is methylotrophic yeast (it can grow on methanol). Like other methylotrophic species such as Hansenula polymorpha and Pichia pastoris, it provides an excellent platform for producing heterologous proteins. Yields in a multigram range of a secreted foreign protein have been reported. A computational method, IPRO, recently predicted mutations that experimentally switched the cofactor specificity of Candida boidinii xylose reductase from NADPH to NADH. See, e.g., Mattanovich et al., 2012, Methods Mol Biol. 824:329-58; Khoury et al., 2009, Protein Sci. 18(10):2125-38.

### Hansenula Polymorpha (Pichia Angusta)

Hansenula polymorpha is methylotrophic yeast (see Candida boidinii). It can furthermore grow on a wide range of other substrates; it is thermo-tolerant and can assimilate nitrate (see also, Kluyveromyces lactis). It has been applied to producing hepatitis B vaccines, insulin and interferon alpha-2a for the treatment of hepatitis C, furthermore to a range of technical enzymes. See, e.g., Xu et al., 2014, Virol Sin. 29(6):403-9.

### Candida Krusei (Issatchenkia Orientalis)

Candida krusei , scientific name Issatchenkia orientalis, is widely used in chocolate production. C. krusei is used to remove the bitter taste of and break down cacao beans. In addition to this species involvement in chocolate production, C. krusei is commonly found in the immunocompromised as a fungal nosocomial pathogen (see Mastromarino et al., New Microbiolgica, 36:229-238; 2013)

### Kluyveromyces Lactis

Kluyveromyces lactis is yeast regularly applied to the production of kefir. It can grow on several sugars, most importantly on lactose which is present in milk and whey. It has successfully been applied among others for producing chymosin (an enzyme that is usually present in the stomach of calves) for producing cheese. Production takes place in fermenters on a 40,000 L scale. See, e.g., van Ooyen et al., 2006, FEMS Yeast Res. 6(3):381-92.

### Pichia Pastoris

Pichia pastoris is methylotrophic yeast (see Candida boidinii and Hansenula polymorpha). It is also commonly referred to as Komagataella pastoris. It provides an efficient platform for producing foreign proteins. Platform elements are available as a kit and it is worldwide used in academia for producing proteins. Strains have been engineered that can produce complex human N-glycan (yeast glycans are similar but not identical to those found in humans). See, e.g., Piirainen et al., 2014, N Biotechnol. 31(6):532-7.

### Scheffersomyces Stipitis

Scheffersomyces stipitis also known as Pichia stipitis is a homothallic yeast found in haploid form. Commonly used instead of S. cerevisiae due to its enhanced respiratory capacity that results from and alternative respiratory system (see Papini et al., Microbial Cell Factories, 11:136 (2012)).

In some embodiments, a microorganism can be an insect cell such as Drosophilia, specifically, Drosophilia melanogaster.

In some embodiments, a microorganism can be an algal cell such as, for example but not limited to, Blakeslea trispora, Dunaliella salina, Haematococcus pluvialis, Chlorella sp.,

In some embodiments, a microorganism can be a cyanobacterial cell such as, for example but not limited to, Blakeslea trispora, Dunaliella salina, Haematococcus pluvialis, Chlorella sp., Undaria pinnatifida, Sargassum, Laminaria japonica, and Scenedesmus almeriensis.

In some embodiments, a microorganism can be a bacterial cell. Examples of bacteria include, but are not limited to, the genera Bacillus (e.g., B. subtilis, B. amyloliquefaciens, B. licheniformis, B. puntis, B. megaterium, B. halodurans, B. pumilus), Acinetobacter, Nocardia, Xanthobacter, Escherichia (e.g., E. coli), Streptomyces, Erwinia, Klebsiella, Serratia (e.g., S. marcessans), Pseudomonas (e.g., P. aeruginosa), Salmonella (e.g., S. typhimurium, and S. typhi). Bacterial cells may also include, but are not limited to, photosynthetic bacteria (e.g., green non-sulfur bacteria (e.g., Choroflexus bacteria (e.g., C. aurantiacus), Chloronema (e.g., C. gigateum), green sulfur bacteria (e.g., Chlorobium bacteria (e.g., C. limicola), Pelodictyon (e.g., P. luteolum), purple sulfur bacteria (e.g., Chromatium (e.g., C. okenii)), and purple non-sulfur bacteria (e.g., Rhode-spirillum (e.g., R. rubrum), Rhodobacter (e.g., R. sphaeroides, R. capsulatus), and Rhodomicrobium bacteria (e.g., R. vanellii)).

### E. Coli

E. coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

It can be appreciated that the recombinant host cell disclosed herein can comprise a plant cell, comprising a plant cell that is grown in a plant, a mammalian cell, an insect cell, a fungal cell from Aspergillus genus; a yeast cell from Saccharomyces (e.g., S. cerevisiae, S. bayanus, S. pastorianus, and S. carlsbergensis), Schizosaccharomyces (e.g., S. pombe), Yarrowia (e.g., Y. lipolytica), Candida (e.g., C. glabrata, C. albicans, C. krusei, C. revkaufi, C. pulcherrima, Candida tropicalis, C. utilis, and C. boidinii), Ashbya (e.g., A. gossypii), Cyberlindnera (e.g., C. jadinii), Pichia (e.g., P. pastoris and P. kudriavzevii), Kluyveromyces (e.g., K. lactis), Hansenual (e.g., H. polymorpha), Arxula (e.g., A. adeninivorans), Xanthophyllomyces (e.g., X. dendrorhous), Issatchenkia (e.g., I. orientali), Torulaspora (e.g., T. franciscae and T. globosa), Geotrichum (e.g., G. candidum and G. klebahni), Zygosaccharomyces (e.g., Z. bisporus and Z. cidri), Yamadazyma (e.g., Y. philogaea), Lanchancea (e.g., L. kluyven), Kodamaea (e.g., K. ohmen), Brettanomyces (e.g., B. anomalus), Trichosporon (e.g., T. aquatile, T. beigelii, and T. dermatis), Debaromyces (e.g., D. hansenuis and D. hansenii), Scheffersomyces (e.g., S. stipis), Rhodosporidium (e.g., R. toruloides), Pachysolen (e.g., P. tannophilus), and Physcomitrella, Rhodotorula, Kazachstania, Gibberella, Agaricus, and Phanerochaete genera; an insect cell including, but not limited to, Drosophilia melanogaster, an algal cell including, but not limited to, Blakeslea trispora, Dunaliella salina, Haematococcus pluvialis, Chlorella sp., Undaria pinnatifida, Sargassum, Laminaria japonica, and Scenedesmus almeriensis species; or a bacterial cell from Bacillus genus (e.g., B. subtilis, B. amyloliquefaciens, B. licheniformis, B. puntis, B. megaterium, B. halodurans, and B. pumilus) Acinetobacter, Nocardia, Xanthobacter genera, Escherichia (e.g., E. coli), Streptomyces, Erwinia, Klebsiella, Serratia (e.g., S. marcessans), Pseudomonas (e.g., P. aeruginosa), Salmonella (e.g., S. typhimurium and S. typhi), and further including, Choroflexus bacteria (e.g., C. aurantiacus), Chloronema (e.g., C. gigateum), green sulfur bacteria (e.g., Chlorobium bacteria (e.g., C. limicola), Pelodictyon (e.g., P. luteolum)), purple sulfur bacteria (e.g., Chromatium (e.g., C. okenii)), and purple non-sulfur bacteria (e.g., Rhode-spirillum (e.g., R. rubrum), Rhodobacter (e.g., R. sphaeroides and R. capsulatus), and Rhodomicrobium bacteria (e.g., R. vanellii).

### Steviol Glycoside Compositions

Steviol glycosides do not necessarily have equivalent performance in different food systems. It is therefore desirable to have the ability to direct the synthesis to steviol glycoside compositions of choice. Recombinant hosts described herein can produce compositions that are selectively enriched for specific steviol glycosides (e.g., RebD or RebM) and have a consistent taste profile. As used herein, the term “enriched” is used to describe a steviol glycoside composition with an increased proportion of a particular steviol glycoside, compared to a steviol glycoside composition (extract) from a Stevia plant. Thus, the recombinant hosts described herein can facilitate the production of compositions that are tailored to meet the sweetening profile desired for a given food product and that have a proportion of each steviol glycoside that is consistent from batch to batch. In some embodiments, hosts described herein do not produce or produce a reduced amount of undesired plant by-products found in Stevia extracts. Thus, steviol glycoside compositions produced by the recombinant hosts described herein are distinguishable from compositions derived from Stevia plants.

The amount of an individual steviol glycoside (e.g., RebA, RebB, RebD, or RebM) accumulated can be from about 1 to about 7,000 mg/L, e.g., about 1 to about 10 mg/L, about 3 to about 10 mg/L, about 5 to about 20 mg/L, about 10 to about 50 mg/L, about 10 to about 100 mg/L, about 25 to about 500 mg/L, about 100 to about 1,500 mg/L, or about 200 to about 1,000 mg/L, at least 1,000 mg/L, at least 1,200 mg/L, at least at least 1,400 mg/L, at least 1,600 mg/L, at least 1,800 mg/L, at least 2,800 mg/L, or at least 7,000 mg/L. In some aspects, the amount of an individual steviol glycoside can exceed 7,000 mg/L. The amount of a combination of steviol glycosides (e.g., RebA, RebB, RebD, or RebM) accumulated can be from about 1 mg/L to about 7,000 mg/L, e.g., about 200 to about 1,500, at least 2,000 mg/L, at least 3,000 mg/L, at least 4,000 mg/L, at least 5,000 mg/L, at least 6,000 mg/L, or at least 7,000 mg/L. In some aspects, the amount of a combination of steviol glycosides can exceed 7,000 mg/L. In general, longer culture times will lead to greater amounts of product. Thus, the recombinant microorganism can be cultured for from 1 day to 7 days, from 1 day to 5 days, from 3 days to 5 days, about 3 days, about 4 days, or about 5 days.

The amount of compounds accumulated by the recombinant host may be reported as a “flux.” For example, the “total flux” may be calculated as a sum (in g/L RebD equivalents) of measured RebA, RebB, RebD, RebE, RebM, 13-SMG, rubusoside, steviol-1,2-bioside, di-glycosylated steviol, tri-glycosylated steviol, tetra-glycosylated steviol, penta-glycosylated steviol, hexa-glycosylated steviol, hepta-glycosylated steviol, copalol, ent-kaurenoic acid, glycosylated ent-kaurenoic acid, glycosylated ent-kaurenol, ent-kaurenal, geranylgeraniol, ent-kaurenal, and ent-kaurene levels. Individual compounds, such as individual steviol glycosides, or groups of compounds, such as the group of steviol glycosides, may be reported as a fraction of total flux. For example, “steviol glycoside/flux” may calculated as ((“total flux”−(geranylgeraniol+copalol+ent-kaurene+glycosylated ent-kaurenol+ent-kaurenol+ent-kaurenal+ent-kaurenoic acid+glycosylated ent-kaurenoic acid)/“total flux”).

It will be appreciated that the various genes and modules discussed herein can be present in two or more recombinant microorganisms rather than a single microorganism. When a plurality of recombinant microorganisms is used, they can be grown in a mixed culture to produce steviol and/or steviol glycosides. For example, a first microorganism can comprise one or more biosynthesis genes for producing a steviol glycoside precursor, while a second microorganism comprises steviol glycoside biosynthesis genes. The product produced by the second, or final microorganism is then recovered. It will also be appreciated that in some embodiments, a recombinant microorganism is grown using nutrient sources other than a culture medium and utilizing a system other than a fermenter.

Alternatively, the two or more microorganisms each can be grown in a separate culture medium and the product of the first culture medium, e.g., steviol, can be introduced into second culture medium to be converted into a subsequent intermediate, or into an end product such as RebA. The product produced by the second, or final microorganism is then recovered. It will also be appreciated that in some embodiments, a recombinant microorganism is grown using nutrient sources other than a culture medium and utilizing a system other than a fermenter.

Steviol glycosides and compositions obtained by the methods disclosed herein can be used to make food products, dietary supplements and sweetener compositions. See, e.g., WO 2011/153378, WO 2013/022989, WO 2014/122227, and WO 2014/122328.

For example, substantially pure steviol or steviol glycoside such as RebM or RebD can be included in food products such as ice cream, carbonated drinks, fruit juices, yogurts, baked goods, chewing gums, hard and soft candies, and sauces. Substantially pure steviol or the steviol glycoside can also be included in non-food products such as pharmaceutical products, medicinal products, dietary supplements and nutritional supplements. Substantially pure steviol or the steviol glycosides may also be included in animal feed products for both the agriculture industry and the companion animal industry. Alternatively, a mixture of steviol and/or steviol glycosides can be made by culturing recombinant microorganisms separately, each producing a specific steviol glycoside, recovering the steviol or the steviol glycoside in substantially pure form from each microorganism and then combining the compounds to obtain a mixture comprising each compound in the desired proportion. The recombinant microorganisms described herein permit more precise and consistent mixtures to be obtained compared to current Stevia products.

In another alternative, a substantially pure steviol or steviol glycoside can be incorporated into a food product along with other sweeteners, e.g., saccharin, dextrose, sucrose, fructose, erythritol, aspartame, sucralose, monatin, or acesulfame potassium. The weight ratio of the steviol or the steviol glycoside relative to other sweeteners can be varied as desired to achieve a satisfactory taste in the final food product. See, e.g., U.S. 2007/0128311. In some embodiments, the steviol or the steviol glycoside may be provided with a flavor (e.g., citrus) as a flavor modulator.

Compositions produced by a recombinant microorganism described herein can be incorporated into food products. For example, a steviol glycoside composition produced by a recombinant microorganism can be incorporated into a food product in an amount ranging from about 20 mg steviol glycoside/kg food product to about 1800 mg steviol glycoside/kg food product on a dry weight basis, depending on the type of steviol glycoside and food product. For example, a steviol glycoside composition produced by a recombinant microorganism can be incorporated into a dessert, cold confectionary (e.g., ice cream), dairy product (e.g., yogurt), or beverage (e.g., a carbonated beverage) such that the food product has a maximum of 500 mg steviol glycoside/kg food on a dry weight basis. A steviol glycoside composition produced by a recombinant microorganism can be incorporated into a baked good (e.g., a biscuit) such that the food product has a maximum of 300 mg steviol glycoside/kg food on a dry weight basis. A steviol glycoside composition produced by a recombinant microorganism can be incorporated into a sauce (e.g., chocolate syrup) or vegetable product (e.g., pickles) such that the food product has a maximum of 1000 mg steviol glycoside/kg food on a dry weight basis. A steviol glycoside composition produced by a recombinant microorganism can be incorporated into bread such that the food product has a maximum of 160 mg steviol glycoside/kg food on a dry weight basis. A steviol glycoside composition produced by a recombinant microorganism, plant, or plant cell can be incorporated into a hard or soft candy such that the food product has a maximum of 1600 mg steviol glycoside/kg food on a dry weight basis. A steviol glycoside composition produced by a recombinant microorganism, plant, or plant cell can be incorporated into a processed fruit product (e.g., fruit juices, fruit filling, jams, and jellies) such that the food product has a maximum of 1000 mg steviol glycoside/kg food on a dry weight basis. In some embodiments, a steviol glycoside composition produced herein is a component of a pharmaceutical composition. See, e.g., Steviol Glycosides Chemical and Technical Assessment 69th JECFA, 2007, prepared by Harriet Wallin, Food Agric. Org.; EFSA Panel on Food Additives and Nutrient Sources added to Food (ANS), “Scientific Opinion on the safety of steviol glycosides for the proposed uses as a food additive,” 2010, EFSA Journal 8(4):1537; U.S. Food and Drug Administration GRAS Notice 323; U.S Food and Drug Administration GRAS Notice 329; WO 2011/037959; WO 2010/146463; WO 2011/046423; and WO 2011/056834.

For example, such a steviol glycoside composition can have from 90-99 weight % RebA and an undetectable amount of Stevia plant-derived contaminants, and be incorporated into a food product at from 25-1600 mg/kg, e.g., 100-500 mg/kg, 25-100 mg/kg, 250-1000 mg/kg, 50-500 mg/kg or 500-1000 mg/kg on a dry weight basis.

Such a steviol glycoside composition can be a RebB-enriched composition having greater than 3 weight % RebB and be incorporated into the food product such that the amount of RebB in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the RebB-enriched composition has an undetectable amount of Stevia plant-derived contaminants.

Such a steviol glycoside composition can be a RebD-enriched composition having greater than 3 weight % RebD and be incorporated into the food product such that the amount of RebD in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the RebD-enriched composition has an undetectable amount of Stevia plant-derived contaminants.

Such a steviol glycoside composition can be a RebE-enriched composition having greater than 3 weight % RebE and be incorporated into the food product such that the amount of RebE in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the RebE-enriched composition has an undetectable amount of Stevia plant-derived contaminants.

Such a steviol glycoside composition can be a RebM-enriched composition having greater than 3 weight % RebM and be incorporated into the food product such that the amount of RebM in the product is from 25-1600 mg/kg, e.g., 100-500 mg/kg, 25-100 mg/kg, 250-1000 mg/kg, 50-500 mg/kg or 500-1000 mg/kg on a dry weight basis. Typically, the RebM-enriched composition has an undetectable amount of Stevia plant-derived contaminants.

In some embodiments, a substantially pure steviol or steviol glycoside is incorporated into a tabletop sweetener or “cup-for-cup” product. Such products typically are diluted to the appropriate sweetness level with one or more bulking agents, e.g., maltodextrins, known to those skilled in the art. Steviol glycoside compositions enriched for RebA, RebB, RebD, RebE, or RebM, can be package in a sachet, for example, at from 10,000 to 30,000 mg steviol glycoside/kg product on a dry weight basis, for tabletop use. In some embodiments, a steviol glycoside produced in vitro, in vivo, or by whole cell bioconversion.

The invention also provides an isolated nucleic acid molecule encoding a polypeptide or a catalytically active portion thereof capable of debranching glycogen comprising a polypeptide or a catalytically active portion thereof having at least 60% sequence identity to the amino acid sequence set forth in SEQ ID NO:157 or a polypeptide or a catalytically active portion thereof capable of synthesizing glucose-1-phosphate comprising a polypeptide or a catalytically active portion thereof having at least 55% sequence identity to the amino acid sequence set forth in SEQ ID NO:159.

In one aspect of the isolated nucleic acids disclosed herein, the nucleic acid is cDNA.

The invention also provides a polypeptide or a catalytically active portion thereof capable of debranching glycogen comprising a polypeptide or a catalytically active portion thereof having at least 60% sequence identity to the amino acid sequence set forth in SEQ ID NO:157 or a polypeptide or a catalytically active portion thereof capable of synthesizing glucose-1-phosphate comprising a polypeptide or a catalytically active portion thereof having at least 55% sequence identity to the amino acid sequence set forth in SEQ ID NO:159.

In one aspect of the polypeptides or the catalytically active portion thereof disclosed herein, the polypeptide or the catalytically active portion thereof is a purified polypeptide or a catalytically active portion thereof.

The invention will be further described in the following examples, which do not limit the scope of the invention described in the claims.

### EXAMPLES

The Examples that follow are illustrative of specific embodiments of the invention, and various uses thereof. They are set forth for explanatory purposes only, and are not to be taken as limiting the invention.

### Example 1: Strain Engineering

Steviol glycoside-producing S. cerevisiae strains were constructed as described in WO 2011/153378, WO 2013/022989, WO 2014/122227, and WO 2014/122328, each of which is incorporated by reference in its entirety. For example, yeast strains comprising and expressing a native gene encoding a YNK1 polypeptide (SEQ ID NO:122, SEQ ID NO:123), a native gene encoding a PGM1 polypeptide (SEQ ID NO:1, SEQ ID NO:2), a native gene encoding a PGM2 polypeptide (SEQ ID NO:118, SEQ ID NO:119), a native gene encoding a UGP1 polypeptide (SEQ ID NO:120, SEQ ID NO:121), a native gene encoding a GDB1 polypeptide (SEQ ID NO:156, SEQ ID NO:157), a native gene encoding a GPH1 polypeptide (SEQ ID NO:158, SEQ ID NO:159), a recombinant gene encoding a GGPPS polypeptide (SEQ ID NO:19, SEQ ID NO:20), a recombinant gene encoding a truncated CDPS polypeptide (SEQ ID NO:39, SEQ ID NO:40), a recombinant gene encoding a KS polypeptide (SEQ ID NO:51, SEQ ID NO:52), a recombinant gene encoding a KO polypeptide (SEQ ID NO:59, SEQ ID NO:60), a recombinant gene encoding a KO polypeptide (SEQ ID NO:63, SEQ ID NO:64), a recombinant gene encoding an ATR2 polypeptide (SEQ ID NO:91, SEQ ID NO:92), a recombinant gene encoding a KAHe1 polypeptide (SEQ ID NO:93, SEQ ID NO:94), a recombinant gene encoding a CPR8 polypeptide (SEQ ID NO:85, SEQ ID NO:86), a recombinant gene encoding a CPR1 polypeptide (SEQ ID NO:77, SEQ ID NO:78), a recombinant gene encoding a UGT76G1 polypeptide (SEQ ID NO:8, SEQ ID NO:9), a recombinant gene encoding a UGT85C2 polypeptide (SEQ ID NO:5/SEQ ID NO:6, SEQ ID NO:7), a recombinant gene encoding a UGT74G1 polypeptide (SEQ ID NO:3, SEQ ID NO:4), a recombinant gene encoding a UGT91d2e-b polypeptide (SEQ ID NO:12, SEQ ID NO:13), a recombinant gene encoding an EUGT11 polypeptide (SEQ ID NO:14, SEQ ID NO:15, SEQ ID NO:16), a recombinant gene encoding a KAH polypeptide (SEQ ID NO:96, SEQ ID NO:97), a recombinant gene encoding a KO polypeptide (SEQ ID NO:117, SEQ ID NO:64), and additional copies of the gene encoding a YNK1 polypeptide (SEQ ID NO:122, SEQ ID NO:123), the gene encoding a PGM1 polypeptide (SEQ ID NO:1, SEQ ID NO:2), the gene encoding a PGM2 polypeptide (SEQ ID NO:118, SEQ ID NO:119), the gene encoding a UGP1 polypeptide (SEQ ID NO:120, SEQ ID NO:121), and the gene encoding an ERC1 transporter polypeptide (i.e., of the MATE family) (SEQ ID NO:160, SEQ ID NO:161) were engineered to accumulate steviol glycosides.

### Example 2: Overexpression of GDB1 and GPH1

A steviol glycoside-producing S. cerevisiae strain as described in Example 1 was transformed with vectors comprising additional copies of the gene encoding a GDB1 polypeptide (SEQ ID NO:156, SEQ ID NO:157), operably linked to a TPI1 promoter (SEQ ID NO:152) and a ADH1 terminator (SEQ ID NO:155) and the gene encoding a GPH1 polypeptide (SEQ ID NO:158, SEQ ID NO:159), operably linked to a pPDC1 promoter (SEQ ID NO:153) and a tCYC1 terminator (SEQ ID NO:154).

Fed-batch fermentation with cultures of the transformed S. cerevisiae strain and a control S. cerevisiae strain (a steviol glycoside-producing S. cerevisiae strain as described in Example 1) was carried out aerobically in 2L fermenters at 30° C. with an approximate 16 h growth phase in minimal medium comprising glucose, ammonium sulfate, trace metals, vitamins, salts, and buffer followed by an approximate 100 h feeding phase with a glucose-comprising defined feed medium. A pH near 6.0 and glucose-limiting conditions were maintained. Extractions of whole culture samples (without cell removal) were performed and extracts were analyzed by LC-UV to determine levels of steviol glycosides.

LC-UV was conducted with an Agilent 1290 instrument comprising a variable wavelength detector (VWD), a thermostatted column compartment (TCC), an autosampler, an autosampler cooling unit, and a binary pump, using SB-C18 rapid resolution high definition (RRHD) 2.1 mm×300 mm, 1.8 μm analytical columns (two 150 mm columns in series; column temperature of 65° C.). Steviol glycosides were separated by a reversed-phase C18 column followed by detection by UV absorbance at 210 mm. Quantification of steviol glycosides was done by comparing the peak area of each analyte to standards of RebA and applying a correction factor for species with differing molar absorptivities. For LC-UV, 0.5 mL cultures were spun down, the supernatant was removed, and the wet weight of the pellets was calculated. The LC-UV results were normalized by pellet wet weight. Total steviol glycoside values of the fed-batch fermentation were calculated based upon the measured levels of steviol glycosides calculated as a sum (in g/L RebD equivalents) of measured RebA, RebB, RebD, RebE, RebM, 13-SMG, rubusoside, steviol-1,2-bioside, di-glycosylated steviol, tri-glycosylated steviol, tetra-glycosylated steviol, penta-glycosylated steviol, hexa-glycosylated steviol, and hepta-glycosylated steviol. Total flux was calculated as a sum (in g/L RebD equivalents) of measured RebA, RebB, RebD, RebE, RebM, 13-SMG, rubusoside, steviol-1,2-bioside, di-glycosylated steviol, tri-glycosylated steviol, tetra-glycosylated steviol, penta-glycosylated steviol, hexa-glycosylated steviol, hepta-glycosylated steviol, copalol, ent-kaurenoic acid, glycosylated ent-kaurenoic acid, glycosylated ent-kaurenol, ent-kaurenal, geranylgeraniol, ent-kaurenal, and ent-kaurene levels. Results are shown in Table 1.

TABLE 1
Steviol Glycoside accumulation by transformed S. cerevisiae
strain and S. cerevisiae control strain.

| | | | | | RebD + | Total | Total |
|---|---|---|---|---|---|---|---|
| | 13-SMG | RebA | RebD | RebM | RebM | SGs | Flux |
| Strains | (g/L) | (g/L) | (g/L) | (g/L) | (g/L) | (g/L) | (g/L) |
| Control | 1.59 | 0.49 | 1.26 | 5.91 | 7.2 | 11.53 | 23.08 |
| +GDB1 | 1.13 | 0.53 | 1.63 | 6.60 | 8.2 | 11.60 | 26.29 |
| +GPH1 | | | | | | | |
| Change | −29% | 8% | 29% | 12% | 14% | 1% | 14% |

End point fermentation titer (120 h) g/L as in RebD equivalent

Percent change in steviol glycoside production (% increase or % decrease) was calculated as follows. The amount of a particular steviol glycoside (e.g., RebM) produced by the control strain (in g/L) was subtracted from the amount of the particular steviol glycoside produced by the experimental strain overexpressing GPH1 and GDB1 (in g/L). That resulting value was then divided by the amount of the particular steviol glycoside produced by the control strain (in g/L) and multiplied by 100. A positive number using this equation signifies a percent increase in a particular steviol glycoside produced by the strain overexpressing GPH1 and GDB1 (in g/L), whereas a negative number using this equation signifies a percent decrease in a particular steviol glycoside (e.g., 13-SMG) produced by the strain overexpressing GPH1 and GDB1 (in g/L).

Overexpression of GPH1 and GDB1 resulted in a 29% decrease in 13-SMG accumulation, and an increase of 8%, 29% and 12% in RebA, RebD and RebM accumulation, respectively, in comparison to the control strain. There was also a 14% increase in RebD+RebM accumulation. Furthermore, there was a 14% increase in total flux accumulated by the strain overexpressing GPH1 and GDB1 genes, compared to the control strain. The total amount of steviol glycosides accumulated changed negligibly. Without being bound by theory, the lack of a significant change in total steviol glycoside accumulation and the decrease in 13-SMG accumulation suggests that overexpression of GPH1 and GDB1 in a steviol glycoside producing recombinant host enhances the flux of glycosylation pathways towards higher molecular weight steviol glycosides, e.g. RebD and RebM, altering the production profile, rather than simply increasing steviol glycoside production, generally.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

TABLE 3
Sequences disclosed herein.

SEQ ID NO: 1
S. cerevisiae

| | |
|---|---|
| atgtcacttc taatagattc tgtaccaaca gttgcttata aggaccaaaa accgggtact | 60 |
| tcaggtttac gtaagaagac caaggttttc atggatgagc ctcattatac tgagaacttc | 120 |
| attcaagcaa caatgcaatc tatccctaat ggctcagagg gaaccacttt agttgttgga | 180 |
| ggagatggtc gtttctacaa cgatgttatc atgaacaaga ttgccgcagt aggtgctgca | 240 |
| aacggtgtca gaaagttagt cattggtcaa ggcggtttac tttcaacacc agctgcttct | 300 |
| catataatta gaacatacga ggaaaagtgt accggtggtg gtatcatatt aactgcctca | 360 |
| cacaacccag gcggtccaga gaatgattta ggtatcaagt ataatttacc taatggtggg | 420 |
| ccagctccag agagtgtcac taacgctatc tgggaagcgt ctaaaaaatt aactcactat | 480 |
| aaaattataa agaacttccc caagttgaat ttgaacaagc ttggtaaaaa ccaaaaatat | 540 |
| ggcccattgt tagtggacat aattgatcct gccaaagcat acgttcaatt tctgaaggaa | 600 |
| atttttgatt ttgacttaat taaaagcttc ttagcgaaac agcgcaaaga caaagggtgg | 660 |
| aagttgttgt ttgactcctt aaatggtatt acaggaccat atggtaaggc tatatttgtt | 720 |
| gatgaatttg gtttaccggc agaggaagtt cttcaaaatt ggcacccttt acctgatttc | 780 |
| ggcggtttac atcccgatcc gaatctaacc tatgcacgaa ctcttgttga cagggttgac | 840 |
| cgcgaaaaaa ttgcctttgg agcagcctcc gatggtgatg gtgataggaa tatgatttac | 900 |
| ggttatggcc ctgctttcgt ttcgccaggt gattctgttg ccattattgc cgaatatgca | 960 |
| cccgaaattc catacttcgc caaacaaggt atttatggct tggcacgttc atttcctaca | 1020 |
| tcctcagcca ttgatcgtgt tgcagcaaaa aagggattaa gatgttacga agttccaacc | 1080 |
| ggctggaaat tcttctgtgc cttatttgat gctaaaaagc tatcaatctg tggtgaagaa | 1140 |
| tccttcggta caggttccaa tcatatcaga gaaaaggacg gtctatgggc cattattgct | 1200 |
| tggttaaata tcttggctat ctaccatagg cgtaaccctg aaaaggaagc ttcgatcaaa | 1260 |
| actattcagg acgaattttg gaacgagtat ggccgtactt tcttcacaag atacgattac | 1320 |
| gaacatatcg aatgcgagca ggccgaaaaa gttgtagctc ttttgagtga atttgtatca | 1380 |
| aggccaaacg tttgtggctc ccacttccca gctgatgagt ctttaaccgt tatcgattgt | 1440 |
| ggtgattttt cgtatagaga tctagatggc tccatctctg aaaatcaagg ccttttcgta | 1500 |
| aagttttcga atgggactaa atttgttttg aggttatccg gcacaggcag ttctggtgca | 1560 |
| acaataagat tatacgtaga aaagtatact gataaaaagg agaactatgg ccaaacagct | 1620 |
| gacgtcttct tgaaacccgt catcaactcc attgtaaaat tcttaagatt taaagaaatt | 1680 |
| ttaggaacag acgaaccaac agtccgcaca tag | 1713 |

SEQ ID NO: 2
S. cerevisiae

| | |
|---|---|
| MSLLIDSVPT VAYKDQKPGT SGLRKKTKVF MDEPHYTENF IQATMQSIPN GSEGTTLVVG | 60 |
| GDGRFYNDVI MNKIAAVGAA NGVRKLVIGQ GGLLSTPAAS HIIRTYEEKC TGGGIILTAS | 120 |
| HNPGGPENDL GIKYNLPNGG PAPESVTNAI WEASKKLTHY KIIKNFPKLN LNKLGKNQKY | 180 |
| GPLLVDIIDP AKAYVQFLKE IFDFDLIKSF LAKQRKDKGW KLLFDSLNGI TGPYGKAIFV | 240 |
| DEFGLPAEEV LQNWHPLPDF GGLHPDPNLT YARTLVDRVD REKIAFGAAS DGDGDRNMIY | 300 |
| GYGPAFVSPG DSVAIIAEYA PEIPYFAKQG IYGLARSFPT SSAIDRVAAK KGLRCYEVPT | 360 |
| GWKFFCALFD AKKLSICGEE SFGTGSNHIR EKDGLWAIIA WLNILAIYHR RNPEKEASIK | 420 |
| TIQDEFWNEY GRTFFTRYDY EHIECEQAEK VVALLSEFVS RPNVCGSHFP ADESLTVIDC | 480 |
| GDFSYRDLDG SISENQGLFV KFSNGTKFVL RLSGTGSSGA TIRLYVEKYT DKKENYGQTA | 540 |
| DVFLKPVINS IVKFLRFKEI LGTDEPTVRT | 570 |

SEQ ID NO: 3
S. rebaudiana

| | |
|---|---|
| atggcagagc aacaaaagat caaaaagtca cctcacgtct tacttattcc atttcctctg | 60 |
| caaggacata tcaacccatt catacaattt gggaaaagat tgattagtaa gggtgtaaag | 120 |
| acaacactgg taaccactat ccacactttg aattctactc tgaaccactc aaatactact | 180 |
| actacaagta tagaaattca agctatatca gacggatgcg atgagggtgg ctttatgtct | 240 |
| gccggtgaat cttacttgga aacattcaag caagtgggat ccaagtctct ggccgatcta | 300 |
| atcaaaaagt tacagagtga aggcaccaca attgacgcca taatctacga ttctatgaca | 360 |
| gagtgggttt tagacgttgc tatcgaattt ggtattgatg gaggttcctt tttcacacaa | 420 |
| gcatgtgttg tgaattctct atactaccat gtgcataaag ggttaatctc tttaccattg | 480 |
| ggtgaaactg tttcagttcc aggttttcca gtgttacaac gttgggaaac cccattgatc | 540 |
| ttacaaaatc atgaacaaat acaatcacct tggtcccaga tgttgtttgg tcaattcgct | 600 |
| aacatcgatc aagcaagatg ggtctttact aattcattct ataagttaga ggaagaggta | 660 |
| attgaatgga ctaggaagat ctggaatttg aaagtcattg gtccaacatt gccatcaatg | 720 |
| tatttggaca aaagacttga tgatgataaa gataatggtt tcaatttgta caaggctaat | 780 |
| catcacgaat gtatgaattg gctggatgac aaaccaaagg aatcagttgt atatgttgct | 840 |
| ttcggctctc ttgttaaaca tggtccagaa caagttgagg agattacaag agcacttata | 900 |
| gactctgacg taaacttttt gtgggtcatt aagcacaaag aggaggggaa actgccagaa | 960 |
| aacctttctg aagtgataaa gaccggaaaa ggtctaatcg ttgcttggtg taaacaattg | 1020 |
| gatgttttag ctcatgaatc tgtaggctgt tttgtaacac attgcggatt caactctaca | 1080 |
| ctagaagcca tttccttagg cgtacctgtc gttgcaatgc ctcagttctc cgatcagaca | 1140 |
| accaacgcta aacttttgga cgaaatacta ggggtgggtg tcagagttaa agcagacgag | 1200 |
| aatggtatcg tcagaagagg gaacctagct tcatgtatca aaatgatcat ggaagaggaa | 1260 |
| agaggagtta tcataaggaa aaacgcagtt aagtggaagg atcttgcaaa ggttgccgtc | 1320 |
| catgaaggcg gctcttcaga taatgatatt gttgaatttg tgtccgaact aatcaaagcc | 1380 |
| taa | 1383 |

SEQ ID NO: 4
S. rebaudiana

| | |
|---|---|
| MAEQQKIKKS PHVLLIPFPL QGHINPFIQF GKRLISKGVK TTLVTTIHTL NSTLNHSNTT | 60 |
| TTSIEIQAIS DGCDEGGFMS AGESYLETFK QVGSKSLADL IKKLQSEGTT IDAIIYDSMT | 120 |
| EWVLDVAIEF GIDGGSFFTQ ACVVNSLYYH VHKGLISLPL GETVSVPGFP VLQRWETPLI | 180 |
| LQNHEQIQSP WSQMLFGQFA NIDQARWVFT NSFYKLEEEV IEWTRKIWNL KVIGPTLPSM | 240 |
| YLDKRLDDDK DNGFNLYKAN HHECMNWLDD KPKESVVYVA FGSLVKHGPE QVEEITRALI | 300 |
| DSDVNFLWVI KHKEEGKLPE NLSEVIKTGK GLIVAWCKQL DVLAHESVGC FVTHCGFNST | 360 |
| LEAISLGVPV VAMPQFSDQT TNAKLLDEIL GVGVRVKADE NGIVRRGNLA SCIKMIMEEE | 420 |
| RGVIIRKNAV KWKDLAKVAV HEGGSSDNDI VEFVSELIKA | 460 |

SEQ ID NO: 5
S. rebaudiana

| | |
|---|---|
| atggatgcaa tggctacaac tgagaagaaa ccacacgtca tcttcatacc atttccagca | 60 |
| caaagccaca ttaaagccat gctcaaacta gcacaacttc tccaccacaa aggactccag | 120 |
| ataaccttcg tcaacaccga cttcatccac aaccagtttc ttgaatcatc gggcccacat | 180 |
| tgtctagacg gtgcaccggg tttccggttc gaaaccattc cggatggtgt ttctcacagt | 240 |
| ccggaagcga gcatcccaat cagagaatca ctcttgagat ccattgaaac caacttcttg | 300 |
| gatcgtttca ttgatcttgt aaccaaactt ccggatcctc cgacttgtat tatctcagat | 360 |
| gggttcttgt cggttttcac aattgacgct gcaaaaaagc ttggaattcc ggtcatgatg | 420 |
| tattggacac ttgctgcctg tgggttcatg ggtttttacc atattcattc tctcattgag | 480 |
| aaaggatttg caccacttaa agatgcaagt tacttgacaa atgggtattt ggacaccgtc | 540 |
| attgattggg ttccgggaat ggaaggcatc cgtctcaagg atttcccgct ggactggagc | 600 |
| actgacctca atgacaaagt tttgatgttc actacggaag ctcctcaaag gtcacacaag | 660 |
| gtttcacatc atattttcca cacgttcgat gagttggagc ctagtattat aaaaactttg | 720 |
| tcattgaggt ataatcacat ttacaccatc ggcccactgc aattacttct tgatcaaata | 780 |
| cccgaagaga aaaagcaaac tggaattacg agtctccatg gatacagttt agtaaaagaa | 840 |
| gaaccagagt gtttccagtg gcttcagtct aaagaaccaa attccgtcgt ttatgtaaat | 900 |
| tttggaagta ctacagtaat gtctttagaa gacatgacgg aatttggttg gggacttgct | 960 |
| aatagcaacc attatttcct ttggatcatc cgatcaaact tggtgatagg ggaaaatgca | 1020 |
| gttttgcccc ctgaacttga ggaacatata aagaaaagag gctttattgc tagctggtgt | 1080 |
| tcacaagaaa aggtcttgaa gcacccttcg gttggagggt tcttgactca ttgtgggtgg | 1140 |
| ggatcgacca tcgagagctt gtctgctggg gtgccaatga tatgctggcc ttattcgtgg | 1200 |
| gaccagctga ccaactgtag gtatatatgc aaagaatggg aggttgggct cgagatggga | 1260 |
| accaaagtga aacgagatga agtcaagagg cttgtacaag agttgatggg agaaggaggt | 1320 |
| cacaaaatga ggaacaaggc taaagattgg aaagaaaagg ctcgcattgc aatagctcct | 1380 |
| aacggttcat cttctttgaa catagacaaa atggtcaagg aaatcaccgt gctagcaaga | 1440 |
| aactagttac aaagttgttt cacattgtgc tttctattta agatgtaact ttgttctaat | 1500 |
| ttaatattgt ctagatgtat tgaaccataa gtttagttgg tctcaggaat tgatttttaa | 1560 |
| tgaaataatg gtcattaggg gtgagt | 1586 |

SEQ ID NO: 6
Artificial Sequence

| | |
|---|---|
| atggatgcaa tggcaactac tgagaaaaag cctcatgtga tcttcattcc atttcctgca | 60 |
| caatctcaca taaaggcaat gctaaagtta gcacaactat tacaccataa gggattacag | 120 |
| ataactttcg tgaataccga cttcatccat aatcaatttc tggaatctag tggccctcat | 180 |
| tgtttggacg gagccccagg gtttagattc gaaacaattc ctgacggtgt ttcacattcc | 240 |
| ccagaggcct ccatcccaat aagagagagt ttactgaggt caatagaaac caactttttg | 300 |
| gatcgtttca ttgacttggt cacaaaactt ccagacccac caacttgcat aatctctgat | 360 |
| ggctttctgt cagtgtttac tatcgacgct gccaaaaagt tgggtatccc agttatgatg | 420 |
| tactggactc ttgctgcatg cggtttcatg ggtttctatc acatccattc tcttatcgaa | 480 |
| aagggttttg ctccactgaa agatgcatca tacttaacca acggctacct ggatactgtt | 540 |
| attgactggg taccaggtat ggaaggtata agacttaaag attttccttt ggattggtct | 600 |
| acagacctta atgataaagt attgatgttt actacagaag ctccacaaag atctcataag | 660 |
| gtttcacatc atatctttca cacctttgat gaattggaac catcaatcat caaaaccttg | 720 |
| tctctaagat acaatcatat ctacactatt ggtccattac aattacttct agatcaaatt | 780 |
| cctgaagaga aaaagcaaac tggtattaca tccttacacg gctactcttt agtgaaagag | 840 |
| gaaccagaat gttttcaatg gctacaaagt aaagagccta attctgtggt ctacgtcaac | 900 |
| ttcggaagta caacagtcat gtccttggaa gatatgactg aatttggttg gggccttgct | 960 |
| aattcaaatc attactttct atggattatc aggtccaatt tggtaatagg ggaaaacgcc | 1020 |
| gtattacctc cagaattgga ggaacacatc aaaaagagag gtttcattgc ttcctggtgt | 1080 |
| tctcaggaaa aggtattgaa acatccttct gttggtggtt tccttactca ttgcggttgg | 1140 |
| ggctctacaa tcgaatcact aagtgcagga gttccaatga tttgttggcc atattcatgg | 1200 |
| gaccaactta caaattgtag gtatatctgt aaagagtggg aagttggatt agaaatggga | 1260 |
| acaaaggtta aacgtgatga agtgaaaaga ttggttcagg agttgatggg ggaaggtggc | 1320 |
| cacaagatga gaaacaaggc caaagattgg aaggaaaaag ccagaattgc tattgctcct | 1380 |
| aacgggtcat cctctctaaa cattgataag atggtcaaag agattacagt cttagccaga | 1440 |
| aactaa | 1446 |

SEQ ID NO: 7
S. rebaudiana

| | |
|---|---|
| MDAMATTEKK PHVIFIPFPA QSHIKAMLKL AQLLHHKGLQ ITFVNTDFIH NQFLESSGPH | 60 |
| CLDGAPGFRF ETIPDGVSHS PEASIPIRES LLRSIETNFL DRFIDLVTKL PDPPTCIISD | 120 |
| GFLSVFTIDA AKKLGIPVMM YWTLAACGFM GFYHIHSLIE KGFAPLKDAS YLTNGYLDTV | 180 |
| IDWVPGMEGI RLKDFPLDWS TDLNDKVLMF TTEAPQRSHK VSHHIFHTFD ELEPSIIKTL | 240 |
| SLRYNHIYTI GPLQLLLDQI PEEKKQTGIT SLHGYSLVKE EPECFQWLQS KEPNSVVYVN | 300 |
| FGSTTVMSLE DMTEFGWGLA NSNHYFLWII RSNLVIGENA VLPPELEEHI KKRGFIASWC | 360 |
| SQEKVLKHPS VGGFLTHCGW GSTIESLSAG VPMICWPYSW DQLTNCRYIC KEWEVGLEMG | 420 |
| TKVKRDEVKR LVQELMGEGG HKMRNKAKDW KEKARIAIAP NGSSSLNIDK MVKEITVLAR | 480 |
| N | 481 |

SEQ ID NO: 8
Artificial Sequence

| | |
|---|---|
| atggaaaaca agaccgaaac aacagttaga cgtaggcgta gaatcattct gtttccagta | 60 |
| ccttttcaag ggcacatcaa tccaatacta caactagcca acgttttgta ctctaaaggt | 120 |
| ttttctatta caatctttca caccaatttc aacaaaccaa aaacatccaa ttacccacat | 180 |
| ttcacattca gattcatact tgataatgat ccacaagatg aacgtatttc aaacttacct | 240 |
| acccacggtc ctttagctgg aatgagaatt ccaatcatca atgaacatgg tgccgatgag | 300 |
| cttagaagag aattagagtt acttatgttg gcatccgaag aggacgagga agtctcttgt | 360 |
| ctgattactg acgctctatg gtactttgcc caatctgtgg ctgatagttt gaatttgagg | 420 |
| agattggtac taatgacatc cagtctgttt aactttcacg ctcatgttag tttaccacaa | 480 |
| tttgacgaat tgggatactt ggaccctgat gacaagacta ggttagagga acaggcctct | 540 |
| ggttttccta tgttgaaagt caaagatatc aagtctgcct attctaattg gcaaatcttg | 600 |
| aaagagatct taggaaagat gatcaaacag acaaaggctt catctggagt gatttggaac | 660 |
| agtttcaaag agttagaaga gtctgaattg gagactgtaa tcagagaaat tccagcacct | 720 |
| tcattcctga taccattacc aaaacatttg actgcttcct cttcctcttt gttggatcat | 780 |
| gacagaacag tttttcaatg gttggaccaa caaccaccta gttctgtttt gtacgtgtca | 840 |
| tttggtagta cttctgaagt cgatgaaaag gacttccttg aaatcgcaag aggcttagtc | 900 |
| gatagtaagc agtcattcct ttgggtcgtg cgtccaggtt tcgtgaaagg ctcaacatgg | 960 |
| gtcgaaccac ttccagatgg ttttctaggc gaaagaggta gaatagtcaa atgggttcct | 1020 |
| caacaggaag ttttagctca tggcgctatt ggggcattct ggactcattc cggatggaat | 1080 |
| tcaactttag aatcagtatg cgaaggggta cctatgatct tttcagattt tggtcttgat | 1140 |
| caaccactga acgcaagata catgtctgat gttttgaaag tgggtgtata tctagaaaat | 1200 |
| ggctgggaaa ggggtgaaat agctaatgca ataagacgtg ttatggttga tgaagagggg | 1260 |
| gagtatatca gacaaaacgc aagagtgctg aagcaaaagg ccgacgtttc tctaatgaag | 1320 |
| ggaggctctt catacgaatc cttagaatct cttgtttcct acatttcatc actgtaa | 1377 |

SEQ ID NO: 9
S. rebaudiana

| | |
|---|---|
| MENKTETTVR RRRRIILFPV PFQGHINPIL QLANVLYSKG FSITIFHTNF NKPKTSNYPH | 60 |
| FTFRFILDND PQDERISNLP THGPLAGMRI PIINEHGADE LRRELELLML ASEEDEEVSC | 120 |
| LITDALWYFA QSVADSLNLR RLVLMTSSLF NFHAHVSLPQ FDELGYLDPD DKTRLEEQAS | 180 |
| GFPMLKVKDI KSAYSNWQIL KEILGKMIKQ TKASSGVIWN SFKELEESEL ETVIREIPAP | 240 |
| SFLIPLPKHL TASSSSLLDH DRTVFQWLDQ QPPSSVLYVS FGSTSEVDEK DFLEIARGLV | 300 |
| DSKQSFLWVV RPGFVKGSTW VEPLPDGFLG ERGRIVKWVP QQEVLAHGAI GAFWTHSGWN | 360 |
| STLESVCEGV PMIFSDFGLD QPLNARYMSD VLKVGVYLEN GWERGEIANA IRRVMVDEEG | 420 |
| EYIRQNARVL KQKADVSLMK GGSSYESLES LVSYISSL | 458 |

SEQ ID NO: 10
Artificial Sequence

| | |
|---|---|
| atggctacat ctgattctat tgttgatgac aggaagcagt tgcatgtggc tactttccct | 60 |
| tggcttgctt tcggtcatat actgccttac ctacaactat caaaactgat agctgaaaaa | 120 |
| ggacataaag tgtcattcct ttcaacaact agaaacattc aaagattatc ttcccacata | 180 |
| tcaccattga ttaacgtcgt tcaattgaca cttccaagag tacaggaatt accagaagat | 240 |
| gctgaagcta caacagatgt gcatcctgaa gatatccctt acttgaaaaa ggcatccgat | 300 |
| ggattacagc ctgaggtcac tagattcctt gagcaacaca gtccagattg gatcatatac | 360 |
| gactacactc actattggtt gccttcaatt gcagcatcac taggcatttc tagggcacat | 420 |
| ttcagtgtaa ccacaccttg ggccattgct tacatgggtc catccgctga tgctatgatt | 480 |
| aacggcagtg atggtagaac taccgttgaa gatttgacaa ccccaccaaa gtggtttcca | 540 |
| tttccaacta aagtctgttg gagaaaacac gacttagcaa gactggttcc atacaaggca | 600 |
| ccaggaatct cagacggcta tagaatgggt ttagtcctta aagggtctga ctgcctattg | 660 |
| tctaagtgtt accatgagtt tgggacacaa tggctaccac ttttggaaac attacaccaa | 720 |
| gttcctgtcg taccagttgg tctattacct ccagaaatcc ctggtgatga gaaggacgag | 780 |
| acttgggttt caatcaaaaa gtggttagac gggaagcaaa aaggctcagt ggtatatgtg | 840 |
| gcactgggtt ccgaagtttt agtatctcaa acagaagttg tggaacttgc cttaggtttg | 900 |
| gaactatctg gattgccatt tgtctgggcc tacagaaaac caaaaggccc tgcaaagtcc | 960 |
| gattcagttg aattgccaga cggctttgtc gagagaacta gagatagagg gttggtatgg | 1020 |
| acttcatggg ctccacaatt gagaatcctg agtcacgaat ctgtgtgcgg tttcctaaca | 1080 |
| cattgtggtt ctggttctat agttgaagga ctgatgtttg gtcatccact tatcatgttg | 1140 |
| ccaatctttg gtgaccagcc tttgaatgca cgtctgttag aagataaaca agttggaatt | 1200 |
| gaaatcccac gtaatgagga agatggatgt ttaaccaagg agtctgtggc cagatcatta | 1260 |
| cgttccgttg tcgttgaaaa ggaaggcgaa atctacaagg ccaatgcccg tgaactttca | 1320 |
| aagatctaca atgacacaaa agtagagaag gaatatgttt ctcaatttgt agattaccta | 1380 |
| gagaaaaacg ctagagccgt agctattgat catgaatcct aa | 1422 |

SEQ ID NO: 11
S. rebaudiana

| | |
|---|---|
| MATSDSIVDD RKQLHVATFP WLAFGHILPY LQLSKLIAEK GHKVSFLSTT RNIQRLSSHI | 60 |
| SPLINVVQLT LPRVQELPED AEATTDVHPE DIPYLKKASD GLQPEVTRFL EQHSPDWIIY | 120 |
| DYTHYWLPSI AASLGISRAH FSVTTPWAIA YMGPSADAMI NGSDGRTTVE DLTTPPKWFP | 180 |
| FPTKVCWRKH DLARLVPYKA PGISDGYRMG LVLKGSDCLL SKCYHEFGTQ WLPLLETLHQ | 240 |
| VPVVPVGLLP PEIPGDEKDE TWVSIKKWLD GKQKGSVVYV ALGSEVLVSQ TEVVELALGL | 300 |
| ELSGLPFVWA YRKPKGPAKS DSVELPDGFV ERTRDRGLVW TSWAPQLRIL SHESVCGFLT | 360 |
| HCGSGSIVEG LMFGHPLIML PIFGDQPLNA RLLEDKQVGI EIPRNEEDGC LTKESVARSL | 420 |
| RSVVVEKEGE IYKANARELS KIYNDTKVEK EYVSQFVDYL EKNARAVAID HES | 473 |

SEQ ID NO: 12
Artificial Sequence

| | |
|---|---|
| atggctactt ctgattccat cgttgacgat agaaagcaat tgcatgttgc tacttttcca | 60 |
| tggttggctt tcggtcatat tttgccatac ttgcaattgt ccaagttgat tgctgaaaag | 120 |
| ggtcacaagg tttcattctt gtctaccacc agaaacatcc aaagattgtc ctctcatatc | 180 |
| tccccattga tcaacgttgt tcaattgact ttgccaagag tccaagaatt gccagaagat | 240 |
| gctgaagcta ctactgatgt tcatccagaa gatatccctt acttgaaaaa ggcttccgat | 300 |
| ggtttacaac cagaagttac tagattcttg gaacaacatt ccccagattg gatcatctac | 360 |
| gattatactc attactggtt gccatccatt gctgcttcat tgggtatttc tagagcccat | 420 |
| ttctctgtta ctactccatg ggctattgct tatatgggtc catctgctga tgctatgatt | 480 |
| aacggttctg atggtagaac taccgttgaa gatttgacta ctccaccaaa gtggtttcca | 540 |
| tttccaacaa aagtctgttg gagaaaacac gatttggcta gattggttcc atacaaagct | 600 |
| ccaggtattt ctgatggtta cagaatgggt atggttttga aaggttccga ttgcttgttg | 660 |
| tctaagtgct atcatgaatt cggtactcaa tggttgcctt tgttggaaac attgcatcaa | 720 |
| gttccagttg ttccagtagg tttgttgcca ccagaaattc caggtgacga aaaagacgaa | 780 |
| acttgggttt ccatcaaaaa gtggttggat ggtaagcaaa agggttctgt tgtttatgtt | 840 |
| gctttgggtt ccgaagcttt ggtttctcaa accgaagttg ttgaattggc tttgggtttg | 900 |
| gaattgtctg gtttgccatt tgtttgggct tacagaaaac ctaaaggtcc agctaagtct | 960 |
| gattctgttg aattgccaga tggtttcgtt gaaagaacta gagatagagg tttggtttgg | 1020 |
| acttcttggg ctccacaatt gagaattttg tctcatgaat ccgtctgtgg tttcttgact | 1080 |
| cattgtggtt ctggttctat cgttgaaggt ttgatgtttg gtcacccatt gattatgttg | 1140 |
| ccaatctttg gtgaccaacc attgaacgct agattattgg aagataagca agtcggtatc | 1200 |
| gaaatcccaa gaaatgaaga agatggttgc ttgaccaaag aatctgttgc tagatctttg | 1260 |
| agatccgttg tcgttgaaaa agaaggtgaa atctacaagg ctaacgctag agaattgtcc | 1320 |
| aagatctaca acgataccaa ggtcgaaaaa gaatacgttt cccaattcgt tgactacttg | 1380 |
| gaaaagaatg ctagagctgt tgccattgat catgaatctt ga | 1422 |

SEQ ID NO: 13
Artificial Sequence

| | |
|---|---|
| MATSDSIVDD RKQLHVATFP WLAFGHILPY LQLSKLIAEK GHKVSFLSTT RNIQRLSSHI | 60 |
| SPLINVVQLT LPRVQELPED AEATTDVHPE DIPYLKKASD GLQPEVTRFL EQHSPDWIIY | 120 |
| DYTHYWLPSI AASLGISRAH FSVTTPWAIA YMGPSADAMI NGSDGRTTVE DLTTPPKWFP | 180 |
| FPTKVCWRKH DLARLVPYKA PGISDGYRMG MVLKGSDCLL SKCYHEFGTQ WLPLLETLHQ | 240 |
| VPVVPVGLLP PEIPGDEKDE TWVSIKKWLD GKQKGSVVYV ALGSEALVSQ TEVVELALGL | 300 |
| ELSGLPFVWA YRKPKGPAKS DSVELPDGFV ERTRDRGLVW TSWAPQLRIL SHESVCGFLT | 360 |
| HCGSGSIVEG LMFGHPLIML PIFGDQPLNA RLLEDKQVGI EIPRNEEDGC LTKESVARSL | 420 |
| RSVVVEKEGE IYKANARELS KIYNDTKVEK EYVSQFVDYL EKNARAVAID HES | 473 |

SEQ ID NO: 14
O. sativa

| | |
|---|---|
| atggactccg gctactcctc ctcctacgcc gccgccgccg ggatgcacgt cgtgatctgc | 60 |
| ccgtggctcg ccttcggcca cctgctcccg tgcctcgacc tcgcccagcg cctcgcgtcg | 120 |
| cggggccacc gcgtgtcgtt cgtctccacg ccgcggaaca tatcccgcct cccgccggtg | 180 |
| cgccccgcgc tcgcgccgct cgtcgccttc gtggcgctgc cgctcccgcg cgtcgagggg | 240 |
| ctccccgacg gcgccgagtc caccaacgac gtcccccacg acaggccgga catggtcgag | 300 |
| ctccaccgga gggccttcga cgggctcgcc gcgcccttct cggagttctt gggcaccgcg | 360 |
| tgcgccgact gggtcatcgt cgacgtcttc caccactggg ccgcagccgc cgctctcgag | 420 |
| cacaaggtgc catgtgcaat gatgttgttg ggctctgcac atatgatcgc ttccatagca | 480 |
| gacagacggc tcgagcgcgc ggagacagag tcgcctgcgg ctgccgggca gggacgccca | 540 |
| gcggcggcgc caacgttcga ggtggcgagg atgaagttga tacgaaccaa aggctcatcg | 600 |
| ggaatgtccc tcgccgagcg cttctccttg acgctctcga ggagcagcct cgtcgtcggg | 660 |
| cggagctgcg tggagttcga gccggagacc gtcccgctcc tgtcgacgct ccgcggtaag | 720 |
| cctattacct tccttggcct tatgccgccg ttgcatgaag gccgccgcga ggacggcgag | 780 |
| gatgccaccg tccgctggct cgacgcgcag ccggccaagt ccgtcgtgta cgtcgcgcta | 840 |
| ggcagcgagg tgccactggg agtggagaag gtccacgagc tcgcgctcgg gctggagctc | 900 |
| gccgggacgc gcttcctctg ggctcttagg aagcccactg gcgtctccga cgccgacctc | 960 |
| ctccccgccg gcttcgagga gcgcacgcgc ggccgcggcg tcgtggcgac gagatgggtt | 1020 |
| cctcagatga gcatactggc gcacgccgcc gtgggcgcgt tcctgaccca ctgcggctgg | 1080 |
| aactcgacca tcgaggggct catgttcggc cacccgctta tcatgctgcc gatcttcggc | 1140 |
| gaccagggac cgaacgcgcg gctaatcgag gcgaagaacg ccggattgca ggtggcaaga | 1200 |
| aacgacggcg atggatcgtt cgaccgagaa ggcgtcgcgg cggcgattcg tgcagtcgcg | 1260 |
| gtggaggaag aaagcagcaa agtgtttcaa gccaaagcca agaagctgca ggagatcgtc | 1320 |
| gcggacatgg cctgccatga gaggtacatc gacggattca ttcagcaatt gagatcttac | 1380 |
| aaggattga | 1389 |

SEQ ID NO: 15
Artificial Sequence

| | |
|---|---|
| atggatagtg gctactcctc atcttatgct gctgccgctg gtatgcacgt tgtgatctgc | 60 |
| ccttggttgg cctttggtca cctgttacca tgtctggatt tagcccaaag actggcctca | 120 |
| agaggccata gagtatcatt tgtgtctact cctagaaata tctctcgttt accaccagtc | 180 |
| agacctgctc tagctcctct agttgcattc gttgctcttc cacttccaag agtagaagga | 240 |
| ttgccagacg gcgctgaatc tactaatgac gtaccacatg atagacctga catggtcgaa | 300 |
| ttgcatagaa gagcctttga tggattggca gctccatttt ctgagttcct gggcacagca | 360 |
| tgtgcagact gggttatagt cgatgtattt catcactggg ctgctgcagc cgcattggaa | 420 |
| cataaggtgc cttgtgctat gatgttgtta gggtcagcac acatgatcgc atccatagct | 480 |
| gatagaagat tggaaagagc tgaaacagaa tccccagccg cagcaggaca aggtaggcca | 540 |
| gctgccgccc caacctttga agtggctaga atgaaattga ttcgtactaa aggtagttca | 600 |
| gggatgagtc ttgctgaaag gttttctctg acattatcta gatcatcatt agttgtaggt | 660 |
| agatcctgcg tcgagttcga acctgaaaca gtacctttac tatctacttt gagaggcaaa | 720 |
| cctattactt tccttggtct aatgcctcca ttacatgaag gaaggagaga agatggtgaa | 780 |
| gatgctactg ttaggtggtt agatgcccaa cctgctaagt ctgttgttta cgttgcattg | 840 |
| ggttctgagg taccactagg ggtggaaaag gtgcatgaat tagcattagg acttgagctg | 900 |
| gccggaacaa gattcctttg ggctttgaga aaaccaaccg gtgtttctga cgccgacttg | 960 |
| ctaccagctg ggttcgaaga gagaacaaga ggccgtggtg tcgttgctac tagatgggtc | 1020 |
| ccacaaatga gtattctagc tcatgcagct gtaggggcct ttctaaccca ttgcggttgg | 1080 |
| aactcaacaa tagaaggact gatgtttggt catccactta ttatgttacc aatctttggc | 1140 |
| gatcagggac ctaacgcaag attgattgag gcaaagaacg caggtctgca ggttgcacgt | 1200 |
| aatgatggtg atggttcctt tgatagagaa ggcgttgcag ctgccatcag agcagtcgcc | 1260 |
| gttgaggaag agtcatctaa agttttccaa gctaaggcca aaaaattaca agagattgtg | 1320 |
| gctgacatgg cttgtcacga aagatacatc gatggtttca tccaacaatt gagaagttat | 1380 |
| aaagactaa | 1389 |

SEQ ID NO: 16
O. sativa

| | |
|---|---|
| MDSGYSSSYA AAAGMHVVIC PWLAFGHLLP CLDLAQRLAS RGHRVSFVST PRNISRLPPV | 60 |
| RPALAPLVAF VALPLPRVEG LPDGAESTND VPHDRPDMVE LHRRAFDGLA APFSEFLGTA | 120 |
| CADWVIVDVF HHWAAAAALE HKVPCAMMLL GSAHMIASIA DRRLERAETE SPAAAGQGRP | 180 |
| AAAPTFEVAR MKLIRTKGSS GMSLAERFSL TLSRSSLVVG RSCVEFEPET VPLLSTLRGK | 240 |
| PITFLGLMPP LHEGRREDGE DATVRWLDAQ PAKSVVYVAL GSEVPLGVEK VHELALGLEL | 300 |
| AGTRFLWALR KPTGVSDADL LPAGFEERTR GRGVVATRWV PQMSILAHAA VGAFLTHCGW | 360 |
| NSTIEGLMFG HPLIMLPIFG DQGPNARLIE AKNAGLQVAR NDGDGSFDRE GVAAAIRAVA | 420 |
| VEEESSKVFQ AKAKKLQEIV ADMACHERYI DGFIQQLRSY KD | 462 |

SEQ ID NO: 17
Artificial Sequence

| | |
|---|---|
| MDSGYSSSYA AAAGMHVVIC PWLAFGHLLP CLDLAQRLAS RGHRVSFVST PRNISRLPPV | 60 |
| RPALAPLVAF VALPLPRVEG LPDGAESTND VPHDRPDMVE LHRRAFDGLA APFSEFLGTA | 120 |
| CADWVIVDVF HHWAAAAALE HKVPCAMMLL GSAHMIASIA DRRLERAETE SPAAAGQGRP | 180 |
| AAAPTFEVAR MKLIRTKGSS GMSLAERFSL TLSRSSLVVG RSCVEFEPET VPLLSTLRGK | 240 |
| PITFLGLLPP EIPGDEKDET WVSIKKWLDG KQKGSVVYVA LGSEALVSQT EVVELALGLE | 300 |
| LSGLPFVWAY RKPKGPAKSD SVELPDGFVE RTRDRGLVWT SWAPQLRILS HESVCGFLTH | 360 |
| CGSGSIVEGL MFGHPLIMLP IFGDQPLNAR LLEDKQVGIE IARNDGDGSF DREGVAAAIR | 420 |
| AVAVEEESSK VFQAKAKKLQ EIVADMACHE RYIDGFIQQL RSYKD | 465 |

SEQ ID NO: 18
Artificial Sequence

| | |
|---|---|
| MATSDSIVDD RKQLHVATFP WLAFGHILPY LQLSKLIAEK GHKVSFLSTT RNIQRLSSHI | 60 |
| SPLINVVQLT LPRVQELPED AEATTDVHPE DIPYLKKASD GLQPEVTRFL EQHSPDWIIY | 120 |
| DYTHYWLPSI AASLGISRAH FSVTTPWAIA YMGPSADAMI NGSDGRTTVE DLTTPPKWFP | 180 |
| FPTKVCWRKH DLARLVPYKA PGISDGYRMG MVLKGSDCLL SKCYHEFGTQ WLPLLETLHQ | 240 |
| VPVVPVGLMP PLHEGRREDG EDATVRWLDA QPAKSVVYVA LGSEVPLGVE KVHELALGLE | 300 |
| LAGTRFLWAL RKPTGVSDAD LLPAGFEERT RGRGVVATRW VPQMSILAHA AVGAFLTHCG | 360 |
| WNSTIEGLMF GHPLIMLPIF GDQGPNARLI EAKNAGLQVP RNEEDGCLTK ESVARSLRSV | 420 |
| VVEKEGEIYK ANARELSKIY NDTKVEKEYV SQFVDYLEKN ARAVAIDHES | 470 |

SEQ ID NO: 19
Artificial Sequence

| | |
|---|---|
| atggctttgg taaacccaac cgctcttttc tatggtacct ctatcagaac aagacctaca | 60 |
| aacttactaa atccaactca aaagctaaga ccagtttcat catcttcctt accttctttc | 120 |
| tcatcagtta gtgcgattct tactgaaaaa catcaatcta atccttctga gaacaacaat | 180 |
| ttgcaaactc atctagaaac tcctttcaac tttgatagtt atatgttgga aaaagtcaac | 240 |
| atggttaacg aggcgcttga tgcatctgtc ccactaaaag acccaatcaa aatccatgaa | 300 |
| tccatgagat actctttatt ggcaggcggt aagagaatca gaccaatgat gtgtattgca | 360 |
| gcctgcgaaa tagtcggagg taatatcctt aacgccatgc cagccgcatg tgccgtggaa | 420 |
| atgattcata ctatgtcttt ggtgcatgac gatcttccat gtatggataa tgatgacttc | 480 |
| agaagaggta aacctatttc acacaaggtc tacggggagg aaatggcagt attgaccggc | 540 |
| gatgctttac taagtttatc tttcgaacat atagctactg ctacaaaggg tgtatcaaag | 600 |
| gatagaatcg tcagagctat aggggagttg gcccgttcag ttggctccga aggtttagtg | 660 |
| gctggacaag ttgtagatat cttgtcagag ggtgctgatg ttggattaga tcacctagaa | 720 |
| tacattcaca tccacaaaac agcaatgttg cttgagtcct cagtagttat tggcgctatc | 780 |
| atgggaggag gatctgatca gcagatcgaa aagttgagaa aattcgctag atctattggt | 840 |
| ctactattcc aagttgtgga tgacattttg gatgttacaa aatctaccga agagttgggg | 900 |
| aaaacagctg gtaaggattt gttgacagat aagacaactt acccaaagtt gttaggtata | 960 |
| gaaaagtcca gagaatttgc cgaaaaactt aacaaggaag cacaagagca attaagtggc | 1020 |
| tttgatagac gtaaggcagc tcctttgatc gcgttagcca actacaatgc gtaccgtcaa | 1080 |
| aattga | 1086 |

SEQ ID NO: 20
S. rebaudiana

| | |
|---|---|
| MALVNPTALF YGTSIRTRPT NLLNPTQKLR PVSSSSLPSF SSVSAILTEK HQSNPSENNN | 60 |
| LQTHLETPFN FDSYMLEKVN MVNEALDASV PLKDPIKIHE SMRYSLLAGG KRIRPMMCIA | 120 |
| ACEIVGGNIL NAMPAACAVE MIHTMSLVHD DLPCMDNDDF RRGKPISHKV YGEEMAVLTG | 180 |
| DALLSLSFEH IATATKGVSK DRIVRAIGEL ARSVGSEGLV AGQVVDILSE GADVGLDHLE | 240 |
| YIHIHKTAML LESSVVIGAI MGGGSDQQIE KLRKFARSIG LLFQVVDDIL DVTKSTEELG | 300 |
| KTAGKDLLTD KTTYPKLLGI EKSREFAEKL NKEAQEQLSG FDRRKAAPLI ALANYNAYRQ | 360 |
| N | 361 |

SEQ ID NO: 21
Artificial Sequence

| | |
|---|---|
| atggctgagc aacaaatatc taacttgctg tctatgtttg atgcttcaca tgctagtcag | 60 |
| aaattagaaa ttactgtcca aatgatggac acataccatt acagagaaac gcctccagat | 120 |
| tcctcatctt ctgaaggcgg ttcattgtct agatacgacg agagaagagt ctctttgcct | 180 |
| ctcagtcata atgctgcctc tccagatatt gtatcacaac tatgtttttc cactgcaatg | 240 |
| tcttcagagt tgaatcacag atggaaatct caaagattaa aggtggccga ttctccttac | 300 |
| aactatatcc taacattacc atcaaaagga attagaggtg cctttatcga ttccctgaac | 360 |
| gtatggttgg aggttccaga ggatgaaaca tcagtcatca aggaagttat tggtatgctc | 420 |
| cacaactctt cattaatcat tgatgacttc caagataatt ctccacttag aagaggaaag | 480 |
| ccatctaccc atacagtctt cggccctgcc caggctatca atactgctac ttacgttata | 540 |
| gttaaagcaa tcgaaaagat acaagacata gtgggacacg atgcattggc agatgttacg | 600 |
| ggtactatta caactatttt ccaaggtcag gccatggact tgtggtggac agcaaatgca | 660 |
| atcgttccat caatacagga atacttactt atggtaaacg ataaaaccgg tgctctcttt | 720 |
| agactgagtt tggagttgtt agctctgaat tccgaagcca gtatttctga ctctgcttta | 780 |
| gaaagtttat ctagtgctgt ttccttgcta ggtcaatact tccaaatcag agacgactat | 840 |
| atgaacttga tcgataacaa gtatacagat cagaaaggct tctgcgaaga tcttgatgaa | 900 |
| ggcaagtact cactaacact tattcatgcc ctccaaactg attcatccga tctactgacc | 960 |
| aacatccttt caatgagaag agtgcaagga aagttaacgg cacaaaagag atgttggttc | 1020 |
| tggaaatga | 1029 |

SEQ ID NO: 22
G. fujikuroi

| | |
|---|---|
| MAEQQISNLL SMFDASHASQ KLEITVQMMD TYHYRETPPD SSSSEGGSLS RYDERRVSLP | 60 |
| LSHNAASPDI VSQLCFSTAM SSELNHRWKS QRLKVADSPY NYILTLPSKG IRGAFIDSLN | 120 |
| VWLEVPEDET SVIKEVIGML HNSSLIIDDF QDNSPLRRGK PSTHTVFGPA QAINTATYVI | 180 |
| VKAIEKIQDI VGHDALADVT GTITTIFQGQ AMDLWWTANA IVPSIQEYLL MVNDKTGALF | 240 |
| RLSLELLALN SEASISDSAL ESLSSAVSLL GQYFQIRDDY MNLIDNKYTD QKGFCEDLDE | 300 |
| GKYSLTLIHA LQTDSSDLLT NILSMRRVQG KLTAQKRCWF WK | 342 |

SEQ ID NO: 23
Artificial Sequence

| | |
|---|---|
| atggaaaaga ctaaggagaa agcagaacgt atcttgctgg agccatacag atacttatta | 60 |
| caactaccag gaaagcaagt ccgttctaaa ctatcacaag cgttcaatca ctggttaaaa | 120 |
| gttcctgaag ataagttaca aatcattatt gaagtcacag aaatgctaca caatgcttct | 180 |
| ttactgatcg atgatataga ggattcttcc aaactgagaa gaggttttcc tgtcgctcat | 240 |
| tccatatacg gggtaccaag tgtaatcaac tcagctaatt acgtctactt cttgggattg | 300 |
| gaaaaagtat tgacattaga tcatccagac gctgtaaagc tattcaccag acaacttctt | 360 |
| gaattgcatc aaggtcaagg tttggatatc tattggagag acacttatac ttgcccaaca | 420 |
| gaagaggagt acaaagcaat ggttctacaa aagactggcg gtttgttcgg acttgccgtt | 480 |
| ggtctgatgc aacttttctc tgattacaag gaggacttaa agcctctgtt ggataccttg | 540 |
| ggcttgtttt tccagattag agatgactac gctaacttac attcaaagga atattcagaa | 600 |
| aacaaatcat tctgtgaaga tttgactgaa gggaagttta gttttccaac aatccacgcc | 660 |
| atttggtcaa gaccagaatc tactcaagtg caaaacattc tgcgtcagag aacagagaat | 720 |
| attgacatca aaaagtattg tgttcagtac ttggaagatg ttggttcttt tgcttacaca | 780 |
| agacatacac ttagagaatt agaggcaaaa gcatacaagc aaatagaagc ctgtggaggc | 840 |
| aatccttctc tagtggcatt ggttaaacat ttgtccaaaa tgttcaccga ggaaaacaag | 900 |
| taa | 903 |

SEQ ID NO: 24
M. musculus

| | |
|---|---|
| MEKTKEKAER ILLEPYRYLL QLPGKQVRSK LSQAFNHWLK VPEDKLQIII EVTEMLHNAS | 60 |
| LLIDDIEDSS KLRRGFPVAH SIYGVPSVIN SANYVYFLGL EKVLTLDHPD AVKLFTRQLL | 120 |
| ELHQGQGLDI YWRDTYTCPT EEEYKAMVLQ KTGGLFGLAV GLMQLFSDYK EDLKPLLDTL | 180 |
| GLFFQIRDDY ANLHSKEYSE NKSFCEDLTE GKFSFPTIHA IWSRPESTQV QNILRQRTEN | 240 |
| IDIKKYCVQY LEDVGSFAYT RHTLRELEAK AYKQIEACGG NPSLVALVKH LSKMFTEENK | 300 |

SEQ ID NO: 25
Artificial Sequence

| | |
|---|---|
| atggcaagat tctattttct taacgcacta ttgatggtta tctcattaca atcaactaca | 60 |
| gccttcactc cagctaaact tgcttatcca acaacaacaa cagctctaaa tgtcgcctcc | 120 |
| gccgaaactt ctttcagtct agatgaatac ttggcctcta agataggacc tatagagtct | 180 |
| gccttggaag catcagtcaa atccagaatt ccacagaccg ataagatctg cgaatctatg | 240 |
| gcctactctt tgatggcagg aggcaagaga attagaccag tgttgtgtat cgctgcatgt | 300 |
| gagatgttcg gtggatccca agatgtcgct atgcctactg ctgtggcatt agaaatgata | 360 |
| cacacaatgt ctttgattca tgatgatttg ccatccatgg ataacgatga cttgagaaga | 420 |
| ggtaaaccaa caaaccatgt cgttttcggc gaagatgtag ctattcttgc aggtgactct | 480 |
| ttattgtcaa cttccttcga gcacgtcgct agagaaacaa aaggagtgtc agcagaaaag | 540 |
| atcgtggatg ttatcgctag attaggcaaa tctgttggtg ccgagggcct tgctggcggt | 600 |
| caagttatgg acttagaatg tgaagctaaa ccaggtacca cattagacga cttgaaatgg | 660 |
| attcatatcc ataaaaccgc tacattgtta caagttgctg tagcttctgg tgcagttcta | 720 |
| ggtggtgcaa ctcctgaaga ggttgctgca tgcgagttgt ttgctatgaa tataggtctt | 780 |
| gcctttcaag ttgccgacga tatccttgat gtaaccgctt catcagaaga tttgggtaaa | 840 |
| actgcaggca aagatgaagc tactgataag acaacttacc caaagttatt aggattagaa | 900 |
| gagagtaagg catacgcaag acaactaatc gatgaagcca aggaaagttt ggctcctttt | 960 |
| ggagatagag ctgccccttt attggccatt gcagatttca ttattgatag aaagaattga | 1020 |

SEQ ID NO: 26
T. pseudonana

| | |
|---|---|
| MARFYFLNAL LMVISLQSTT AFTPAKLAYP TTTTALNVAS AETSFSLDEY LASKIGPIES | 60 |
| ALEASVKSRI PQTDKICESM AYSLMAGGKR IRPVLCIAAC EMFGGSQDVA MPTAVALEMI | 120 |
| HTMSLIHDDL PSMDNDDLRR GKPTNHVVFG EDVAILAGDS LLSTSFEHVA RETKGVSAEK | 180 |
| IVDVIARLGK SVGAEGLAGG QVMDLECEAK PGTTLDDLKW IHIHKTATLL QVAVASGAVL | 240 |
| GGATPEEVAA CELFAMNIGL AFQVADDILD VTASSEDLGK TAGKDEATDK TTYPKLLGLE | 300 |
| ESKAYARQLI DEAKESLAPF GDRAAPLLAI ADFIIDRKN | 339 |

SEQ ID NO: 27
Artificial Sequence

| | |
|---|---|
| atgcacttag caccacgtag agtccctaga ggtagaagat caccacctga cagagttcct | 60 |
| gaaagacaag gtgccttggg tagaagacgt ggagctggct ctactggctg tgcccgtgct | 120 |
| gctgctggtg ttcaccgtag aagaggagga ggcgaggctg atccatcagc tgctgtgcat | 180 |
| agaggctggc aagccggtgg tggcaccggt ttgcctgatg aggtggtgtc taccgcagcc | 240 |
| gccttagaaa tgtttcatgc ttttgcttta atccatgatg atatcatgga tgatagtgca | 300 |
| actagaagag gctccccaac tgttcacaga gccctagctg atcgtttagg cgctgctctg | 360 |
| gacccagatc aggccggtca actaggagtt tctactgcta tcttggttgg agatctggct | 420 |
| ttgacatggt ccgatgaatt gttatacgct ccattgactc cacatagact ggcagcagta | 480 |
| ctaccattgg taacagctat gagagctgaa accgttcatg gccaatatct tgatataact | 540 |
| agtgctagaa gacctgggac cgatacttct cttgcattga gaatagccag atataagaca | 600 |
| gcagcttaca caatggaacg tccactgcac attggtgcag ccctggctgg ggcaagacca | 660 |
| gaactattag cagggctttc agcatacgcc ttgccagctg gagaagcctt ccaattggca | 720 |
| gatgacctgc taggcgtctt cggtgatcca agacgtacag ggaaacctga cctagatgat | 780 |
| cttagaggtg gaaagcatac tgtcttagtc gccttggcaa gagaacatgc cactccagaa | 840 |
| cagagacaca cattggatac attattgggt acaccaggtc ttgatagaca aggcgcttca | 900 |
| agactaagat gcgtattggt agcaactggt gcaagagccg aagccgaaag acttattaca | 960 |
| gagagaagag atcaagcatt aactgcattg aacgcattaa cactgccacc tcctttagct | 1020 |
| gaggcattag caagattgac attagggtct acagctcatc ctgcctaa | 1068 |

SEQ ID NO: 28
S. clavuligerus

| | |
|---|---|
| MHLAPRRVPR GRRSPPDRVP ERQGALGRRR GAGSTGCARA AAGVHRRRGG GEADPSAAVH | 60 |
| RGWQAGGGTG LPDEVVSTAA ALEMFHAFAL IHDDIMDDSA TRRGSPTVHR ALADRLGAAL | 120 |
| DPDQAGQLGV STAILVGDLA LTWSDELLYA PLTPHRLAAV LPLVTAMRAE TVHGQYLDIT | 180 |
| SARRPGTDTS LALRIARYKT AAYTMERPLH IGAALAGARP ELLAGLSAYA LPAGEAFQLA | 240 |
| DDLLGVFGDP RRTGKPDLDD LRGGKHTVLV ALAREHATPE QRHTLDTLLG TPGLDRQGAS | 300 |
| RLRCVLVATG ARAEAERLIT ERRDQALTAL NALTLPPPLA EALARLTLGS TAHPA | 355 |

SEQ ID NO: 29
Artificial Sequence

| | |
|---|---|
| atgtcatatt tcgataacta cttcaatgag atagttaatt ccgtgaacga catcattaag | 60 |
| tcttacatct ctggcgacgt accaaaacta tacgaagcct cctaccattt gtttacatca | 120 |
| ggaggaaaga gactaagacc attgatcctt acaatttctt ctgatctttt cggtggacag | 180 |
| agagaaagag catactatgc tggcgcagca atcgaagttt tgcacacatt cactttggtt | 240 |
| cacgatgata tcatggatca agataacatt cgtagaggtc ttcctactgt acatgtcaag | 300 |
| tatggcctac ctttggccat tttagctggt gacttattgc atgcaaaagc ctttcaattg | 360 |
| ttgactcagg cattgagagg tctaccatct gaaactatca tcaaggcgtt tgatatcttt | 420 |
| acaagatcta tcattatcat atcagaaggt caagctgtcg atatggaatt cgaagataga | 480 |
| attgatatca aggaacaaga gtatttggat atgatatctc gtaaaaccgc tgccttattc | 540 |
| tcagcttctt cttccattgg ggcgttgata gctggagcta atgataacga tgtgagatta | 600 |
| atgtccgatt tcggtacaaa tcttgggatc gcatttcaaa ttgtagatga tatacttggt | 660 |
| ttaacagctg atgaaaaaga gctaggaaaa cctgttttca gtgatatcag agaaggtaaa | 720 |
| aagaccatat tagtcattaa gactttagaa ttgtgtaagg aagacgagaa aaagattgtg | 780 |
| ttaaaagcgc taggcaacaa gtcagcatca aaggaagagt tgatgagttc tgctgacata | 840 |
| atcaaaaagt actcattgga ttacgcctac aacttagctg agaaatacta caaaaacgcc | 900 |
| atcgattctc taaatcaagt ttcaagtaaa agtgatattc cagggaaggc attgaaatat | 960 |
| cttgctgaat tcaccatcag aagacgtaag taa | 993 |

SEQ ID NO: 30
S. acidocaldarius

| | |
|---|---|
| MSYFDNYFNE IVNSVNDIIK SYISGDVPKL YEASYHLFTS GGKRLRPLIL TISSDLFGGQ | 60 |
| RERAYYAGAA IEVLHTFTLV HDDIMDQDNI RRGLPTVHVK YGLPLAILAG DLLHAKAFQL | 120 |
| LTQALRGLPS ETIIKAFDIF TRSIIIISEG QAVDMEFEDR IDIKEQEYLD MISRKTAALF | 180 |
| SASSSIGALI AGANDNDVRL MSDFGTNLGI AFQIVDDILG LTADEKELGK PVFSDIREGK | 240 |
| KTILVIKTLE LCKEDEKKIV LKALGNKSAS KEELMSSADI IKKYSLDYAY NLAEKYYKNA | 300 |
| IDSLNQVSSK SDIPGKALKY LAEFTIRRRK | 330 |

SEQ ID NO: 31
Artificial Sequence

| | |
|---|---|
| atggtcgcac aaactttcaa cctggatacc tacttatccc aaagacaaca acaagttgaa | 60 |
| gaggccctaa gtgctgctct tgtgccagct tatcctgaga gaatatacga agctatgaga | 120 |
| tactccctcc tggcaggtgg caaaagatta agacctatct tatgtttagc tgcttgcgaa | 180 |
| ttggcaggtg gttctgttga acaagccatg ccaactgcgt gtgcacttga aatgatccat | 240 |
| acaatgtcac taattcatga tgacctgcca gccatggata acgatgattt cagaagagga | 300 |
| aagccaacta atcacaaggt gttcggggaa gatatagcca tcttagcggg tgatgcgctt | 360 |
| ttagcttacg cttttgaaca tattgcttct caaacaagag gagtaccacc tcaattggtg | 420 |
| ctacaagtta ttgctagaat cggacacgcc gttgctgcaa caggcctcgt tggaggccaa | 480 |
| gtcgtagacc ttgaatctga aggtaaagct atttccttag aaacattgga gtatattcac | 540 |
| tcacataaga ctggagcctt gctggaagca tcagttgtct caggcggtat tctcgcaggg | 600 |
| gcagatgaag agcttttggc cagattgtct cattacgcta gagatatagg cttggctttt | 660 |
| caaatcgtcg atgatatcct ggatgttact gctacatctg aacagttggg gaaaaccgct | 720 |
| ggtaaagacc aggcagccgc aaaggcaact tatccaagtc tattgggttt agaagcctct | 780 |
| agacagaaag cggaagagtt gattcaatct gctaaggaag ccttaagacc ttacggttca | 840 |
| caagcagagc cactcctagc gctggcagac ttcatcacac gtcgtcagca ttaa | 894 |

SEQ ID NO: 32
Synechococcus sp.

| | |
|---|---|
| MVAQTFNLDT YLSQRQQQVE EALSAALVPA YPERIYEAMR YSLLAGGKRL RPILCLAACE | 60 |
| LAGGSVEQAM PTACALEMIH TMSLIHDDLP AMDNDDFRRG KPTNHKVFGE DIAILAGDAL | 120 |
| LAYAFEHIAS QTRGVPPQLV LQVIARIGHA VAATGLVGGQ VVDLESEGKA ISLETLEYIH | 180 |
| SHKTGALLEA SVVSGGILAG ADEELLARLS HYARDIGLAF QIVDDILDVT ATSEQLGKTA | 240 |
| GKDQAAAKAT YPSLLGLEAS RQKAEELIQS AKEALRPYGS QAEPLLALAD FITRRQH | 297 |

SEQ ID NO: 33
Artificial Sequence

| | |
|---|---|
| atgaaaaccg ggtttatctc accagcaaca gtatttcatc acagaatctc accagcgacc | 60 |
| actttcagac atcacttatc acctgctact acaaactcta caggcattgt cgccttaaga | 120 |
| gacatcaact tcagatgtaa agcagtttct aaagagtact ctgatctgtt gcagaaagat | 180 |
| gaggcttctt tcacaaaatg ggacgatgac aaggtgaaag atcatcttga taccaacaaa | 240 |
| aacttatacc caaatgatga gattaaggaa tttgttgaat cagtaaaggc tatgttcggt | 300 |
| agtatgaatg acggggagat aaacgtctct gcatacgata ctgcatgggt tgctttggtt | 360 |
| caagatgtcg atggatcagg tagtcctcag ttcccttctt ctttagaatg gattgccaac | 420 |
| aatcaattgt cagatggatc atggggagat catttgctgt tctcagctca cgatagaatc | 480 |
| atcaacacat tagcatgcgt tattgcactt acaagttgga atgttcatcc ttctaagtgt | 540 |
| gaaaaaggtt tgaattttct gagagaaaac atttgcaaat tagaagatga aaacgcagaa | 600 |
| catatgccaa ttggttttga agtaacattc ccatcactaa ttgatatcgc gaaaaagttg | 660 |
| aacattgaag tacctgagga tactccagca cttaaagaga tctacgcacg tagagatatc | 720 |
| aagttaacta agatcccaat ggaagttctt cacaaggtac ctactacttt gttacattct | 780 |
| ttggaaggaa tgcctgattt ggagtgggaa aaactgttaa agctacaatg taaagatggt | 840 |
| agtttcttgt tttccccatc tagtaccgca ttcgccctaa tgcaaacaaa agatgagaaa | 900 |
| tgcttacagt atctaacaaa tatcgtcact aagttcaacg gtggcgtgcc taatgtgtac | 960 |
| ccagtcgatt tgtttgaaca tatttgggtt gttgatagac tgcagagatt ggggattgcc | 1020 |
| agatacttca aatcagagat aaaagattgt gtagagtata tcaataagta ctggaccaaa | 1080 |
| aatggaattt gttgggctag aaatactcac gttcaagata tcgatgatac agccatggga | 1140 |
| ttcagagtgt tgagagcgca cggttatgac gtcactccag atgtttttag acaatttgaa | 1200 |
| aaagatggta aattcgtttg ctttgcaggg caatcaacac aagccgtgac aggaatgttt | 1260 |
| aacgtttaca gagcctctca aatgttgttc ccaggggaga gaattttgga agatgccaaa | 1320 |
| aagttctctt acaattactt aaaggaaaag caaagtacca acgaattgct ggataaatgg | 1380 |
| ataatcgcta aagatctacc tggtgaagtt ggttatgctc tggatatccc atggtatgct | 1440 |
| tccttaccaa gattggaaac tcgttattac cttgaacaat acggcggtga agatgatgtc | 1500 |
| tggataggca agacattata cagaatgggt tacgtgtcca ataacacata tctagaaatg | 1560 |
| gcaaagctgg attacaataa ctatgttgca gtccttcaat tagaatggta cacaatacaa | 1620 |
| caatggtacg tcgatattgg tatagagaag ttcgaatctg acaacatcaa gtcagtcctg | 1680 |

SEQ ID NO: 34
S. rebaudiana

| | |
|---|---|
| MKTGFISPAT VFHHRISPAT TFRHHLSPAT TNSTGIVALR DINFRCKAVS KEYSDLLQKD | 60 |
| EASFTKWDDD KVKDHLDTNK NLYPNDEIKE FVESVKAMFG SMNDGEINVS AYDTAWVALV | 120 |
| QDVDGSGSPQ FPSSLEWIAN NQLSDGSWGD HLLFSAHDRI INTLACVIAL TSWNVHPSKC | 180 |
| EKGLNFLREN ICKLEDENAE HMPIGFEVTF PSLIDIAKKL NIEVPEDTPA LKEIYARRDI | 240 |
| KLTKIPMEVL HKVPTTLLHS LEGMPDLEWE KLLKLQCKDG SFLFSPSSTA FALMQTKDEK | 300 |
| CLQYLTNIVT KFNGGVPNVY PVDLFEHIWV VDRLQRLGIA RYFKSEIKDC VEYINKYWTK | 360 |
| NGICWARNTH VQDIDDTAMG FRVLRAHGYD VTPDVFRQFE KDGKFVCFAG QSTQAVTGMF | 420 |
| NVYRASQMLF PGERILEDAK KFSYNYLKEK QSTNELLDKW IIAKDLPGEV GYALDIPWYA | 480 |
| SLPRLETRYY LEQYGGEDDV WIGKTLYRMG YVSNNTYLEM AKLDYNNYVA VLQLEWYTIQ | 540 |
| QWYVDIGIEK FESDNIKSVL VSYYLAAASI FEPERSKERI AWAKTTILVD KITSIFDSSQ | 600 |
| SSKEDITAFI DKFRNKSSSK KHSINGEPWH EVMVALKKTL HGFALDALMT HSQDIHPQLH | 660 |
| QAWEMWLTKL QDGVDVTAEL MVQMINMTAG RWVSKELLTH PQYQRLSTVT NSVCHDITKL | 720 |
| HNFKENSTTV DSKVQELVQL VFSDTPDDLD QDMKQTFLTV MKTFYYKAWC DPNTINDHIS | 780 |
| KVFEIVI | 787 |

SEQ ID NO: 35
Artificial Sequence

| | |
|---|---|
| atgcctgatg cacacgatgc tccacctcca caaataagac agagaacact agtagatgag | 60 |
| gctacccaac tgctaactga gtccgcagaa gatgcatggg gtgaagtcag tgtgtcagaa | 120 |
| tacgaaacag caaggctagt tgcccatgct acatggttag gtggacacgc cacaagagtg | 180 |
| gccttccttc tggagagaca acacgaagac gggtcatggg gtccaccagg tggatatagg | 240 |
| ttagtcccta cattatctgc tgttcacgca ttattgacat gtcttgcctc tcctgctcag | 300 |
| gatcatggcg ttccacatga tagactttta agagctgttg acgcaggctt gactgccttg | 360 |
| agaagattgg ggacatctga ctccccacct gatactatag cagttgagct ggttatccca | 420 |
| tctttgctag agggcattca acacttactg gaccctgctc atcctcatag tagaccagcc | 480 |
| ttctctcaac atagaggctc tcttgtttgt cctggtggac tagatgggag aactctagga | 540 |
| gctttgagat cacacgccgc agcaggtaca ccagtaccag gaaaagtctg gcacgcttcc | 600 |
| gagactttgg gcttgagtac cgaagctgct tctcacttgc aaccagccca aggtataatc | 660 |
| ggtggctctg ctgctgccac agcaacatgg ctaaccaggg ttgcaccatc tcaacagtca | 720 |
| gattctgcca gaagatacct tgaggaatta caacacagat actctggccc agttccttcc | 780 |
| attaccccta tcacatactt cgaaagagca tggttattga acaattttgc agcagccggt | 840 |
| gttccttgtg aggctccagc tgctttgttg gattccttag aagcagcact tacaccacaa | 900 |
| ggtgctcctg ctggagcagg attgcctcca gatgctgatg atacagccgc tgtgttgctt | 960 |
| gcattggcaa cacatgggag aggtagaaga ccagaagtac tgatggatta caggactgac | 1020 |
| gggtatttcc aatgctttat tggggaaagg actccatcaa tttcaacaaa cgctcacgta | 1080 |
| ttggaaacat tagggcatca tgtggcccaa catccacaag atagagccag atacggatca | 1140 |
| gccatggata ccgcatcagc ttggctgctg gcagctcaaa agcaagatgg ctcttggtta | 1200 |
| gataaatggc atgcctcacc atactacgct actgtttgtt gcacacaagc cctagccgct | 1260 |
| catgcaagtc ctgcaactgc accagctaga cagagagctg tcagatgggt tttagccaca | 1320 |
| caaagatccg atggcggttg gggtctatgg cattcaactg ttgaagagac tgcttatgcc | 1380 |
| ttacagatct tggccccacc ttctggtggt ggcaatatcc cagtccaaca agcacttact | 1440 |
| agaggcagag caagattgtg tggagccttg ccactgactc ctttatggca tgataaggat | 1500 |
| ttgtatactc cagtaagagt agtcagagct gccagagctg ctgctctgta cactaccaga | 1560 |
| gatctattgt taccaccatt gtaa | 1584 |

SEQ ID NO: 36
S. clavuligerus

| | |
|---|---|
| MPDAHDAPPP QIRQRTLVDE ATQLLTESAE DAWGEVSVSE YETARLVAHA TWLGGHATRV | 60 |
| AFLLERQHED GSWGPPGGYR LVPTLSAVHA LLTCLASPAQ DHGVPHDRLL RAVDAGLTAL | 120 |
| RRLGTSDSPP DTIAVELVIP SLLEGIQHLL DPAHPHSRPA FSQHRGSLVC PGGLDGRTLG | 180 |
| ALRSHAAAGT PVPGKVWHAS ETLGLSTEAA SHLQPAQGII GGSAAATATW LTRVAPSQQS | 240 |
| DSARRYLEEL QHRYSGPVPS ITPITYFERA WLLNNFAAAG VPCEAPAALL DSLEAALTPQ | 300 |
| GAPAGAGLPP DADDTAAVLL ALATHGRGRR PEVLMDYRTD GYFQCFIGER TPSISTNAHV | 360 |
| LETLGHHVAQ HPQDRARYGS AMDTASAWLL AAQKQDGSWL DKWHASPYYA TVCCTQALAA | 420 |
| HASPATAPAR QRAVRWVLAT QRSDGGWGLW HSTVEETAYA LQILAPPSGG GNIPVQQALT | 480 |
| RGRARLCGAL PLTPLWHDKD LYTPVRVVRA ARAAALYTTR DLLLPPL | 527 |

SEQ ID NO: 37
Artificial Sequence

| | |
|---|---|
| atgaacgccc tatccgaaca cattttgtct gaattgagaa gattattgtc tgaaatgagt | 60 |
| gatggcggat ctgttggtcc atctgtgtat gatacggccc aggccctaag attccacggt | 120 |
| aacgtaacag gtagacaaga tgcatatgct tggttgatcg cccagcaaca agcagatgga | 180 |
| ggttggggct ctgccgactt tccactcttt agacatgctc caacatgggc tgcacttctc | 240 |
| gcattacaaa gagctgatcc acttcctggc gcagcagacg cagttcagac cgcaacaaga | 300 |
| ttcttgcaaa gacaaccaga tccatacgct catgccgttc ctgaggatgc ccctattggt | 360 |
| gctgaactga tcttgcctca gttttgtgga gaggctgctt ggttgttggg aggtgtggcc | 420 |
| ttccctagac acccagccct attaccatta agacaggctt gtttagtcaa actgggtgca | 480 |
| gtcgccatgt tgccttcagg acacccattg ctccactcct gggaggcatg gggtacttct | 540 |
| ccaacaacag cctgtccaga cgatgatggt tctataggta tctcaccagc agctacagcc | 600 |
| gcctggagag cccaggctgt gaccagaggc tcaactcctc aagtgggcag agctgacgca | 660 |
| tacttacaaa tggcttcaag agcaacgaga tcaggcatag aaggagtctt ccctaatgtt | 720 |
| tggcctataa acgtattcga accatgctgg tcactgtaca ctctccatct tgccggtctg | 780 |
| ttcgcccatc cagcactggc tgaggctgta agagttatcg ttgctcaact tgaagcaaga | 840 |
| ttgggagtgc atggcctcgg accagcttta cattttgctg ccgacgctga tgatactgca | 900 |
| gttgccttat gcgttctgca tttggctggc agagatcctg cagttgacgc attgagacat | 960 |
| tttgaaattg gtgagctctt tgttacattc ccaggagaga gaaatgctag tgtctctacg | 1020 |
| aacattcacg ctcttcatgc tttgagattg ttaggtaaac cagctgccgg agcaagtgca | 1080 |
| tacgtcgaag caaatagaaa tccacatggt ttgtgggaca acgaaaaatg gcacgtttca | 1140 |
| tggctttatc caactgcaca cgccgttgca gctctagctc aaggcaagcc tcaatggaga | 1200 |
| gatgaaagag cactagccgc tctactacaa gctcaaagag atgatggtgg ttggggagct | 1260 |
| ggtagaggat ccactttcga ggaaaccgcc tacgctcttt tcgctttaca cgttatggac | 1320 |
| ggatctgagg aagccacagg cagaagaaga atcgctcaag tcgtcgcaag agccttagaa | 1380 |
| tggatgctag ctagacatgc cgcacatgga ttaccacaaa caccactctg gattggtaag | 1440 |
| gaattgtact gtcctactag agtcgtaaga gtagctgagc tagctggcct gtggttagca | 1500 |
| ttaagatggg gtagaagagt attagctgaa ggtgctggtg ctgcacctta a | 1551 |

SEQ ID NO: 38
B. japonicum

| | |
|---|---|
| MNALSEHILS ELRRLLSEMS DGGSVGPSVY DTAQALRFHG NVTGRQDAYA WLIAQQQADG | 60 |
| GWGSADFPLF RHAPTWAALL ALQRADPLPG AADAVQTATR FLQRQPDPYA HAVPEDAPIG | 120 |
| AELILPQFCG EAAWLLGGVA FPRHPALLPL RQACLVKLGA VAMLPSGHPL LHSWEAWGTS | 180 |
| PTTACPDDDG SIGISPAATA AWRAQAVTRG STPQVGRADA YLQMASRATR SGIEGVFPNV | 240 |
| WPINVFEPCW SLYTLHLAGL FAHPALAEAV RVIVAQLEAR LGVHGLGPAL HFAADADDTA | 300 |
| VALCVLHLAG RDPAVDALRH FEIGELFVTF PGERNASVST NIHALHALRL LGKPAAGASA | 360 |
| YVEANRNPHG LWDNEKWHVS WLYPTAHAVA ALAQGKPQWR DERALAALLQ AQRDDGGWGA | 420 |
| GRGSTFEETA YALFALHVMD GSEEATGRRR IAQVVARALE WMLARHAAHG LPQTPLWIGK | 480 |
| ELYCPTRVVR VAELAGLWLA LRWGRRVLAE GAGAAP | 516 |

SEQ ID NO: 39
Artificial Sequence

| | |
|---|---|
| atggttttgt cttcttcttg tactacagta ccacacttat cttcattagc tgtcgtgcaa | 60 |
| cttggtcctt ggagcagtag gattaaaaag aaaaccgata ctgttgcagt accagccgct | 120 |
| gcaggaaggt ggagaagggc cttggctaga gcacagcaca catcagaatc cgcagctgtc | 180 |
| gcaaagggca gcagtttgac ccctatagtg agaactgacg ctgagtcaag gagaacaaga | 240 |
| tggccaaccg atgacgatga cgccgaacct ttagtggatg agatcagggc aatgcttact | 300 |
| tccatgtctg atggtgacat ttccgtgagc gcatacgata cagcctgggt cggattggtt | 360 |
| ccaagattag acggcggtga aggtcctcaa tttccagcag ctgtgagatg gataagaaat | 420 |
| aaccagttgc ctgacggaag ttggggcgat gccgcattat tctctgccta tgacaggctt | 480 |
| atcaataccc ttgcctgcgt tgtaactttg acaaggtggt ccctagaacc agagatgaga | 540 |
| ggtagaggac tatctttttt gggtaggaac atgtggaaat tagcaactga agatgaagag | 600 |
| tcaatgccta ttggcttcga attagcattt ccatctttga tagagcttgc taagagccta | 660 |
| ggtgtccatg acttccctta tgatcaccag gccctacaag gaatctactc ttcaagagag | 720 |
| atcaaaatga agaggattcc aaaagaagtg atgcataccg ttccaacatc aatattgcac | 780 |
| agtttggagg gtatgcctgg cctagattgg gctaaactac ttaaactaca gagcagcgac | 840 |
| ggaagttttt tgttctcacc agctgccact gcatatgctt taatgaatac cggagatgac | 900 |
| aggtgtttta gctacatcga tagaacagta aagaaattca acggcggcgt ccctaatgtt | 960 |
| tatccagtgg atctatttga acatatttgg gccgttgata gacttgaaag attaggaatc | 1020 |
| tccaggtact tccaaaagga gatcgaacaa tgcatggatt atgtaaacag gcattggact | 1080 |
| gaggacggta tttgttgggc aaggaactct gatgtcaaag aggtggacga cacagctatg | 1140 |
| gcctttagac ttcttaggtt gcacggctac agcgtcagtc ctgatgtgtt taaaaacttc | 1200 |
| gaaaaggacg gtgaattttt cgcatttgtc ggacagtcta atcaagctgt taccggtatg | 1260 |
| tacaacttaa acagagcaag ccagatatcc ttcccaggcg aggatgtgct tcatagagct | 1320 |
| ggtgccttct catatgagtt cttgaggaga aaagaagcag agggagcttt gagggacaag | 1380 |
| tggatcattt ctaaagatct acctggtgaa gttgtgtata ctttggattt tccatggtac | 1440 |
| ggcaacttac ctagagtcga ggccagagac tacctagagc aatacggagg tggtgatgac | 1500 |
| gtttggattg gcaagacatt gtataggatg ccacttgtaa acaatgatgt atatttggaa | 1560 |
| ttggcaagaa tggatttcaa ccactgccag gctttgcatc agttagagtg gcaaggacta | 1620 |
| aaaagatggt atactgaaaa taggttgatg gactttggtg tcgcccaaga agatgccctt | 1680 |
| agagcttatt ttcttgcagc cgcatctgtt tacgagcctt gtagagctgc cgagaggctt | 1740 |
| gcatgggcta gagccgcaat actagctaac gccgtgagca cccacttaag aaatagccca | 1800 |
| tcattcagag aaaggttaga gcattctctt aggtgtagac ctagtgaaga gacagatggc | 1860 |
| tcctggttta actcctcaag tggctctgat gcagttttag taaaggctgt cttaagactt | 1920 |
| actgattcat tagccaggga agcacagcca atccatggag gtgacccaga agatattata | 1980 |
| cacaagttgt taagatctgc ttgggccgag tgggttaggg aaaaggcaga cgctgccgat | 2040 |
| agcgtgtgca atggtagttc tgcagtagaa caagagggat caagaatggt ccatgataaa | 2100 |
| cagacctgtc tattattggc tagaatgatc gaaatttctg ccggtagggc agctggtgaa | 2160 |
| gcagccagtg aggacggcga tagaagaata attcaattaa caggctccat ctgcgacagt | 2220 |
| cttaagcaaa aaatgctagt ttcacaggac cctgaaaaaa atgaagagat gatgtctcac | 2280 |
| gtggatgacg aattgaagtt gaggattaga gagttcgttc aatatttgct tagactaggt | 2340 |
| gaaaaaaaga ctggatctag cgaaaccagg caaacatttt taagtatagt gaaatcatgt | 2400 |
| tactatgctg ctcattgccc acctcatgtc gttgatagac acattagtag agtgattttc | 2460 |
| gagccagtaa gtgccgcaaa gtaaccgcgg | 2490 |

SEQ ID NO: 40
Z. mays

| | |
|---|---|
| MVLSSSCTTV PHLSSLAVVQ LGPWSSRIKK KTDTVAVPAA AGRWRRALAR AQHTSESAAV | 60 |
| AKGSSLTPIV RTDAESRRTR WPTDDDDAEP LVDEIRAMLT SMSDGDISVS AYDTAWVGLV | 120 |
| PRLDGGEGPQ FPAAVRWIRN NQLPDGSWGD AALFSAYDRL INTLACVVTL TRWSLEPEMR | 180 |
| GRGLSFLGRN MWKLATEDEE SMPIGFELAF PSLIELAKSL GVHDFPYDHQ ALQGIYSSRE | 240 |
| IKMKRIPKEV MHTVPTSILH SLEGMPGLDW AKLLKLQSSD GSFLFSPAAT AYALMNTGDD | 300 |
| RCFSYIDRTV KKFNGGVPNV YPVDLFEHIW AVDRLERLGI SRYFQKEIEQ CMDYVNRHWT | 360 |
| EDGICWARNS DVKEVDDTAM AFRLLRLHGY SVSPDVFKNF EKDGEFFAFV GQSNQAVTGM | 420 |
| YNLNRASQIS FPGEDVLHRA GAFSYEFLRR KEAEGALRDK WIISKDLPGE VVYTLDFPWY | 480 |
| GNLPRVEARD YLEQYGGGDD VWIGKTLYRM PLVNNDVYLE LARMDFNHCQ ALHQLEWQGL | 540 |
| KRWYTENRLM DFGVAQEDAL RAYFLAAASV YEPCRAAERL AWARAAILAN AVSTHLRNSP | 600 |
| SFRERLEHSL RCRPSEETDG SWFNSSSGSD AVLVKAVLRL TDSLAREAQP IHGGDPEDII | 660 |
| HKLLRSAWAE WVREKADAAD SVCNGSSAVE QEGSRMVHDK QTCLLLARMI EISAGRAAGE | 720 |
| AASEDGDRRI IQLTGSICDS LKQKMLVSQD PEKNEEMMSH VDDELKLRIR EFVQYLLRLG | 780 |
| EKKTGSSETR QTFLSIVKSC YYAAHCPPHV VDRHISRVIF EPVSAAK | 827 |

SEQ ID NO: 41
Artificial Sequence

| | |
|---|---|
| cttcttcact aaatacttag acagagaaaa cagagctttt taaagccatg tctcttcagt | 60 |
| atcatgttct aaactccatt ccaagtacaa cctttctcag ttctactaaa acaacaatat | 120 |
| cttcttcttt ccttaccatc tcaggatctc ctctcaatgt cgctagagac aaatccagaa | 180 |
| gcggttccat acattgttca aagcttcgaa ctcaagaata cattaattct caagaggttc | 240 |
| aacatgattt gcctctaata catgagtggc aacagcttca aggagaagat gctcctcaga | 300 |
| ttagtgttgg aagtaatagt aatgcattca aagaagcagt gaagagtgtg aaaacgatct | 360 |
| tgagaaacct aacggacggg gaaattacga tatcggctta cgatacagct tgggttgcat | 420 |
| tgatcgatgc cggagataaa actccggcgt ttccctccgc cgtgaaatgg atcgccgaga | 480 |
| accaactttc cgatggttct tggggagatg cgtatctctt ctcttatcat gatcgtctca | 540 |
| tcaataccct tgcatgcgtc gttgctctaa gatcatggaa tctctttcct catcaatgca | 600 |
| acaaaggaat cacgtttttc cgggaaaata ttgggaagct agaagacgaa aatgatgagc | 660 |
| atatgccaat cggattcgaa gtagcattcc catcgttgct tgagatagct cgaggaataa | 720 |
| acattgatgt accgtacgat tctccggtct taaaagatat atacgccaag aaagagctaa | 780 |
| agcttacaag gataccaaaa gagataatgc acaagatacc aacaacattg ttgcatagtt | 840 |
| tggaggggat gcgtgattta gattgggaaa agctcttgaa acttcaatct caagacggat | 900 |
| ctttcctctt ctctccttcc tctaccgctt ttgcattcat gcagacccga gacagtaact | 960 |
| gcctcgagta tttgcgaaat gccgtcaaac gtttcaatgg aggagttccc aatgtctttc | 1020 |
| ccgtggatct tttcgagcac atatggatag tggatcggtt acaacgttta gggatatcga | 1080 |
| gatactttga agaagagatt aaagagtgtc ttgactatgt ccacagatat tggaccgaca | 1140 |
| atggcatatg ttgggctaga tgttcccatg tccaagacat cgatgataca gccatggcat | 1200 |
| ttaggctctt aagacaacat ggataccaag tgtccgcaga tgtattcaag aactttgaga | 1260 |
| aagagggaga gtttttctgc tttgtggggc aatcaaacca agcagtaacc ggtatgttca | 1320 |
| acctataccg ggcatcacaa ttggcgtttc caagggaaga gatattgaaa aacgccaaag | 1380 |
| agttttctta taattatctg ctagaaaaac gggagagaga ggagttgatt gataagtgga | 1440 |
| ttataatgaa agacttacct ggcgagattg ggtttgcgtt agagattcca tggtacgcaa | 1500 |
| gcttgcctcg agtagagacg agattctata ttgatcaata tggtggagaa aacgacgttt | 1560 |
| ggattggcaa gactctttat aggatgccat acgtgaacaa taatggatat ctggaattag | 1620 |
| caaaacaaga ttacaacaat tgccaagctc agcatcagct cgaatgggac atattccaaa | 1680 |
| agtggtatga agaaaatagg ttaagtgagt ggggtgtgcg cagaagtgag cttctcgagt | 1740 |
| gttactactt agcggctgca actatatttg aatcagaaag gtcacatgag agaatggttt | 1800 |
| gggctaagtc aagtgtattg gttaaagcca tttcttcttc ttttggggaa tcctctgact | 1860 |
| ccagaagaag cttctccgat cagtttcatg aatacattgc caatgctcga cgaagtgatc | 1920 |
| atcactttaa tgacaggaac atgagattgg accgaccagg atcggttcag gccagtcggc | 1980 |
| ttgccggagt gttaatcggg actttgaatc aaatgtcttt tgaccttttc atgtctcatg | 2040 |
| gccgtgacgt taacaatctc ctctatctat cgtggggaga ttggatggaa aaatggaaac | 2100 |
| tatatggaga tgaaggagaa ggagagctca tggtgaagat gataattcta atgaagaaca | 2160 |
| atgacctaac taacttcttc acccacactc acttcgttcg tctcgcggaa atcatcaatc | 2220 |
| gaatctgtct tcctcgccaa tacttaaagg caaggagaaa cgatgagaag gagaagacaa | 2280 |
| taaagagtat ggagaaggag atggggaaaa tggttgagtt agcattgtcg gagagtgaca | 2340 |
| catttcgtga cgtcagcatc acgtttcttg atgtagcaaa agcattttac tactttgctt | 2400 |
| tatgtggcga tcatctccaa actcacatct ccaaagtctt gtttcaaaaa gtctagtaac | 2460 |
| ctcatcatca tcatcgatcc attaacaatc agtggatcga tgtatccata gatgcgtgaa | 2520 |
| taatatttca tgtagagaag gagaacaaat tagatcatgt agggttatca | 2570 |

SEQ ID NO: 42
A. thaliana

| | |
|---|---|
| MSLQYHVLNS IPSTTFLSST KTTISSSFLT ISGSPLNVAR DKSRSGSIHC SKLRTQEYIN | 60 |
| SQEVQHDLPL IHEWQQLQGE DAPQISVGSN SNAFKEAVKS VKTILRNLTD GEITISAYDT | 120 |
| AWVALIDAGD KTPAFPSAVK WIAENQLSDG SWGDAYLFSY HDRLINTLAC VVALRSWNLF | 180 |
| PHQCNKGITF FRENIGKLED ENDEHMPIGF EVAFPSLLEI ARGINIDVPY DSPVLKDIYA | 240 |
| KKELKLTRIP KEIMHKIPTT LLHSLEGMRD LDWEKLLKLQ SQDGSFLFSP SSTAFAFMQT | 300 |
| RDSNCLEYLR NAVKRFNGGV PNVFPVDLFE HIWIVDRLQR LGISRYFEEE IKECLDYVHR | 360 |
| YWTDNGICWA RCSHVQDIDD TAMAFRLLRQ HGYQVSADVF KNFEKEGEFF CFVGQSNQAV | 420 |
| TGMFNLYRAS QLAFPREEIL KNAKEFSYNY LLEKREREEL IDKWIIMKDL PGEIGFALEI | 480 |
| PWYASLPRVE TRFYIDQYGG ENDVWIGKTL YRMPYVNNNG YLELAKQDYN NCQAQHQLEW | 540 |
| DIFQKWYEEN RLSEWGVRRS ELLECYYLAA ATIFESERSH ERMVWAKSSV LVKAISSSFG | 600 |
| ESSDSRRSFS DQFHEYIANA RRSDHHFNDR NMRLDRPGSV QASRLAGVLI GTLNQMSFDL | 660 |
| FMSHGRDVNN LLYLSWGDWM EKWKLYGDEG EGELMVKMII LMKNNDLTNF FTHTHFVRLA | 720 |
| EIINRICLPR QYLKARRNDE KEKTIKSMEK EMGKMVELAL SESDTFRDVS ITFLDVAKAF | 780 |
| YYFALCGDHL QTHISKVLFQ KV | 802 |

SEQ ID NO: 43
Artificial Sequence

| | |
|---|---|
| atgaatttga gtttgtgtat agcatctcca ctattgacca aatctaatag accagctgct | 60 |
| ttatcagcaa ttcatacagc tagtacatcc catggtggcc aaaccaaccc tacgaatctg | 120 |
| ataatcgata cgaccaagga gagaatacaa aaacaattca aaaatgttga aatttcagtt | 180 |
| tcttcttatg atactgcgtg ggttgccatg gttccatcac ctaattctcc aaagtctcca | 240 |
| tgtttcccag aatgtttgaa ttggctgatt aacaaccagt tgaatgatgg atcttggggt | 300 |
| ttagtcaatc acacgcacaa tcacaaccat ccacttttga aagattcttt atcctcaact | 360 |
| ttggcttgca tcgtggccct aaagagatgg aacgtaggtg aggatcagat taacaagggg | 420 |
| cttagtttca ttgaatctaa cttggcttcc gcgactgaaa aatctcaacc atctccaata | 480 |
| ggattcgata tcatctttcc aggtctgtta gagtacgcca aaaatctaga tatcaactta | 540 |
| ctgtctaagc aaactgattt ctcactaatg ttacacaaga gagaattaga acaaaagaga | 600 |
| tgtcattcaa acgaaatgga tggttaccta gcttatatct ctgaaggtct tggtaatctt | 660 |
| tacgattgga atatggtgaa aaagtaccag atgaaaaatg gctcagtttt caattcccct | 720 |
| tctgcaactg cggcagcatt cattaaccat caaaatccag gatgcctgaa ctatttgaat | 780 |
| tcactactag acaaattcgg caacgcagtt ccaactgtat accctcacga tttgtttatc | 840 |
| agattgagta tggtggatac aattgaaaga cttggtatat cccaccactt tagagtcgag | 900 |
| atcaaaaatg ttttggatga gacataccgt tgttgggtgg agagagatga acaaatcttt | 960 |
| atggatgttg tgacgtgcgc gttggccttt agattgttgc gtattaacgg ttacgaagtt | 1020 |
| agtccagatc cacttgccga aattacaaac gaattagctt taaaggatga atacgccgct | 1080 |
| cttgaaacat atcatgcgtc acatatcctt taccaagagg acttatcatc tggaaaacaa | 1140 |
| attcttaaat ctgctgattt cctgaaggaa atcatatcca ctgatagtaa tagactgtcc | 1200 |
| aaactgatcc ataaagaggt tgaaaatgca cttaagttcc ctattaacac cggcttagaa | 1260 |
| cgtattaaca caagacgtaa catccagctt tacaacgtag acaatactag aatcttgaaa | 1320 |
| accacttacc attcttccaa catatcaaac actgattacc taagattagc tgttgaagat | 1380 |
| ttctacacat gtcagtctat ctatagagaa gagctgaaag gattagagag atgggtcgtt | 1440 |
| gagaataagc tagatcaatt gaaatttgcc agacaaaaga cagcttattg ttacttctca | 1500 |
| gttgccgcca ctttatcaag tccagaattg tcagatgcac gtatttcttg ggctaaaaac | 1560 |
| ggaattttga caactgttgt tgatgatttc tttgatattg gcgggacaat cgacgaattg | 1620 |
| acaaacctga ttcaatgcgt tgaaaagtgg aatgtcgatg tcgataaaga ctgttgctca | 1680 |
| gaacatgtta gaatactgtt cttggctctg aaagatgcta tctgttggat cggggatgag | 1740 |
| gctttcaaat ggcaagctag agatgtgacg tctcacgtca ttcaaacctg gctagaactg | 1800 |
| atgaactcta tgttgagaga agcaatttgg actagagatg catacgttcc tacattaaac | 1860 |
| gagtatatgg aaaacgctta tgtctccttt gctttgggtc ctatcgttaa gcctgccata | 1920 |
| tactttgtag gaccaaagct atccgaggaa atcgtcgaat catcagaata ccataacttg | 1980 |
| ttcaagttaa tgtccacaca aggcagatta cttaatgata ttcattcttt caaaagagag | 2040 |
| tttaaggaag gaaagttaaa tgctgttgct ctgcatcttt ctaatggcga aagtggtaaa | 2100 |
| gtcgaagagg aagtagttga ggaaatgatg atgatgatca aaaacaagag aaaggagttg | 2160 |
| atgaaactaa tcttcgaaga gaacggttca attgttccta gagcatgtaa ggatgcattt | 2220 |
| tggaacatgt gtcatgtgct aaactttttc tacgcaaacg acgatggttt tactgggaac | 2280 |
| acaatactag atacagtaaa agacatcata tacaaccctt tggtcttagt aaacgaaaac | 2340 |
| gaggagcaaa gataa | 2355 |

SEQ ID NO: 44
S. rebaudiana

| | |
|---|---|
| MNLSLCIASP LLTKSNRPAA LSAIHTASTS HGGQTNPTNL IIDTTKERIQ KQFKNVEISV | 60 |
| SSYDTAWVAM VPSPNSPKSP CFPECLNWLI NNQLNDGSWG LVNHTHNHNH PLLKDSLSST | 120 |
| LACIVALKRW NVGEDQINKG LSFIESNLAS ATEKSQPSPI GFDIIFPGLL EYAKNLDINL | 180 |
| LSKQTDFSLM LHKRELEQKR CHSNEMDGYL AYISEGLGNL YDWNMVKKYQ MKNGSVFNSP | 240 |
| SATAAAFINH QNPGCLNYLN SLLDKFGNAV PTVYPHDLFI RLSMVDTIER LGISHHFRVE | 300 |
| IKNVLDETYR CWVERDEQIF MDVVTCALAF RLLRINGYEV SPDPLAEITN ELALKDEYAA | 360 |
| LETYHASHIL YQEDLSSGKQ ILKSADFLKE IISTDSNRLS KLIHKEVENA LKFPINTGLE | 420 |
| RINTRRNIQL YNVDNTRILK TTYHSSNISN TDYLRLAVED FYTCQSIYRE ELKGLERWVV | 480 |
| ENKLDQLKFA RQKTAYCYFS VAATLSSPEL SDARISWAKN GILTTVVDDF FDIGGTIDEL | 540 |
| TNLIQCVEKW NVDVDKDCCS EHVRILFLAL KDAICWIGDE AFKWQARDVT SHVIQTWLEL | 600 |
| MNSMLREAIW TRDAYVPTLN EYMENAYVSF ALGPIVKPAI YFVGPKLSEE IVESSEYHNL | 660 |
| FKLMSTQGRL LNDIHSFKRE FKEGKLNAVA LHLSNGESGK VEEEVVEEMM MMIKNKRKEL | 720 |
| MKLIFEENGS IVPRACKDAF WNMCHVLNFF YANDDGFTGN TILDTVKDII YNPLVLVNEN | 780 |
| EEQR | 784 |

SEQ ID NO: 45
Artificial Sequence

| | |
|---|---|
| atgaatctgt ccctttgtat agctagtcca ctgttgacaa aatcttctag accaactgct | 60 |
| ctttctgcaa ttcatactgc cagtactagt catggaggtc aaacaaaccc aacaaatttg | 120 |
| ataatcgata ctactaagga gagaatccaa aagctattca aaaatgttga aatctcagta | 180 |
| tcatcttatg acaccgcatg ggttgcaatg gtgccatcac ctaattcccc aaaaagtcca | 240 |
| tgttttccag agtgcttgaa ttggttaatc aataatcagt taaacgatgg ttcttggggt | 300 |
| ttagtcaacc acactcataa ccacaatcat ccattattga aggactcttt atcatcaaca | 360 |
| ttagcctgta ttgttgcatt gaaaagatgg aatgtaggtg aagatcaaat caacaagggt | 420 |
| ttatcattca tagaatccaa tctagcttct gctaccgaca aatcacaacc atctccaatc | 480 |
| gggttcgaca taatcttccc tggtttgctg gagtatgcca aaaaccttga tatcaactta | 540 |
| ctgtctaaac aaacagattt ctctttgatg ctacacaaaa gagagttaga gcagaaaaga | 600 |
| tgccattcta acgaaattga cgggtactta gcatatatct cagaaggttt gggtaatttg | 660 |
| tatgactgga acatggtcaa aaagtatcag atgaaaaatg gatccgtatt caattctcct | 720 |
| tctgcaactg ccgcagcatt cattaatcat caaaaccctg ggtgtcttaa ctacttgaac | 780 |
| tcactattag ataagtttgg aaatgcagtt ccaacagtct atcctttgga cttgtacatc | 840 |
| agattatcta tggttgacac tatagagaga ttaggtattt ctcatcattt cagagttgag | 900 |
| atcaaaaatg ttttggacga gacatacaga tgttgggtcg aaagagatga gcaaatcttt | 960 |
| atggatgtcg tgacctgcgc tctggctttt agattgctaa ggatacacgg atacaaagta | 1020 |
| tctcctgatc aactggctga gattacaaac gaactggctt tcaaagacga atacgccgca | 1080 |
| ttagaaacat accatgcatc ccaaatactt taccaggaag acctaagttc aggaaaacaa | 1140 |
| atcttgaagt ctgcagattt cctgaaaggc attctgtcta cagatagtaa taggttgtct | 1200 |
| aaattgatac acaaggaagt agaaaacgca ctaaagtttc ctattaacac tggtttagag | 1260 |
| agaatcaata ctaggagaaa cattcagctg tacaacgtag ataatacaag gattcttaag | 1320 |
| accacctacc atagttcaaa catttccaac acctattact taagattagc tgtcgaagac | 1380 |
| ttttacactt gtcaatcaat ctacagagag gagttaaagg gcctagaaag atgggtagtt | 1440 |
| caaaacaagt tggatcaact gaagtttgct agacagaaga cagcatactg ttatttctct | 1500 |
| gttgctgcta ccctttcatc cccagaattg tctgatgcca gaataagttg ggccaaaaat | 1560 |
| ggtattctta caactgtagt cgatgatttc tttgatattg gaggtactat tgatgaactg | 1620 |
| acaaatctta ttcaatgtgt tgaaaagtgg aacgtggatg tagataagga ttgctgcagt | 1680 |
| gaacatgtga gaatactttt cctggctcta aaagatgcaa tatgttggat tggcgacgag | 1740 |
| gccttcaagt ggcaagctag agatgttaca tctcatgtca tccaaacttg gcttgaactg | 1800 |
| atgaactcaa tgctaagaga agcaatctgg acaagagatg catacgttcc aacattgaac | 1860 |
| gaatacatgg aaaacgctta cgtctcattt gccttgggtc ctattgttaa gccagccata | 1920 |
| tactttgttg ggccaaagtt atccgaagag attgttgagt cttccgaata tcataaccta | 1980 |
| ttcaagttaa tgtcaacaca aggcagactt ctgaacgata tccactcctt caaaagagaa | 2040 |
| ttcaaggaag gtaagctaaa cgctgttgct ttgcacttgt ctaatggtga atctggcaaa | 2100 |
| gtggaagagg aagtcgttga ggaaatgatg atgatgatca aaaacaagag aaaggaattg | 2160 |
| atgaaattga ttttcgagga aaatggttca atcgtaccta gagcttgtaa agatgctttt | 2220 |
| tggaatatgt gccatgttct taacttcttt tacgctaatg atgatggctt cactggaaat | 2280 |
| acaatattgg atacagttaa agatatcatc tacaacccac ttgttttggt caatgagaac | 2340 |
| gaggaacaaa gataa | 2355 |

SEQ ID NO: 46
S. rebaudiana

| | |
|---|---|
| MNLSLCIASP LLTKSSRPTA LSAIHTASTS HGGQTNPTNL IIDTTKERIQ KLFKNVEISV | 60 |
| SSYDTAWVAM VPSPNSPKSP CFPECLNWLI NNQLNDGSWG LVNHTHNHNH PLLKDSLSST | 120 |
| LACIVALKRW NVGEDQINKG LSFIESNLAS ATDKSQPSPI GFDIIFPGLL EYAKNLDINL | 180 |
| LSKQTDFSLM LHKRELEQKR CHSNEIDGYL AYISEGLGNL YDWNMVKKYQ MKNGSVFNSP | 240 |
| SATAAAFINH QNPGCLNYLN SLLDKFGNAV PTVYPLDLYI RLSMVDTIER LGISHHFRVE | 300 |
| IKNVLDETYR CWVERDEQIF MDVVTCALAF RLLRIHGYKV SPDQLAEITN ELAFKDEYAA | 360 |
| LETYHASQIL YQEDLSSGKQ ILKSADFLKG ILSTDSNRLS KLIHKEVENA LKFPINTGLE | 420 |
| RINTRRNIQL YNVDNTRILK TTYHSSNISN TYYLRLAVED FYTCQSIYRE ELKGLERWVV | 480 |
| QNKLDQLKFA RQKTAYCYFS VAATLSSPEL SDARISWAKN GILTTVVDDF FDIGGTIDEL | 540 |
| TNLIQCVEKW NVDVDKDCCS EHVRILFLAL KDAICWIGDE AFKWQARDVT SHVIQTWLEL | 600 |
| MNSMLREAIW TRDAYVPTLN EYMENAYVSF ALGPIVKPAI YFVGPKLSEE IVESSEYHNL | 660 |
| FKLMSTQGRL LNDIHSFKRE FKEGKLNAVA LHLSNGESGK VEEEVVEEMM MMIKNKRKEL | 720 |
| MKLIFEENGS IVPRACKDAF WNMCHVLNFF YANDDGFTGN TILDTVKDII YNPLVLVNEN | 780 |
| EEQR | 784 |

SEQ ID NO: 47
Artificial Sequence

| | |
|---|---|
| atggctatgc cagtgaagct aacacctgcg tcattatcct taaaagctgt gtgctgcaga | 60 |
| ttctcatccg gtggccatgc tttgagattc gggagtagtc tgccatgttg gagaaggacc | 120 |
| cctacccaaa gatctacttc ttcctctact actagaccag ctgccgaagt gtcatcaggt | 180 |
| aagagtaaac aacatgatca ggaagctagt gaagcgacta tcagacaaca attacaactt | 240 |
| gtggatgtcc tggagaatat gggaatatcc agacattttg ctgcagagat aaagtgcata | 300 |
| ctagacagaa cttacagatc ttggttacaa agacacgagg aaatcatgct ggacactatg | 360 |
| acatgtgcta tggcttttag aatcctaaga ttgaacggat acaacgtttc atcagatgaa | 420 |
| ctataccacg ttgtagaggc atctggtctg cataattctt tgggtgggta tcttaacgat | 480 |
| accagaacac tacttgaatt acacaaggct tcaacagtta gtatctctga ggatgaatct | 540 |
| atcttagatt caattggctc tagatccaga acattgctta gagaacaatt ggagtctggt | 600 |
| ggcgcactga gaaagccttc tttattcaaa gaggttgaac atgcactgga tggacctttt | 660 |
| tacaccacac ttgatagact tcatcatagg tggaatattg aaaacttcaa cattattgag | 720 |
| caacacatgt tggagactcc atacttatct aaccagcata catcaaggga tatcctagca | 780 |
| ttgtcaatta gagatttttc ctcctcacaa ttcacttatc aacaagagct acagcatctg | 840 |
| gagagttggg ttaaggaatg tagattagat caactacagt tcgcaagaca gaaattagcg | 900 |
| tacttttacc tatcagccgc aggcaccatg ttttctcctg agctttctga tgcgagaaca | 960 |
| ttatgggcca aaaacggggt gttgacaact attgttgatg atttctttga tgttgccggt | 1020 |
| tctaaagagg aattggaaaa cttagtcatg ctggtcgaaa tgtgggatga acatcacaaa | 1080 |
| gttgaattct attctgagca ggtcgaaatc atcttctctt ccatctacga ttctgtcaac | 1140 |
| caattgggtg agaaggcctc tttggttcaa gacagatcaa ttacaaaaca ccttgttgaa | 1200 |
| atatggttag acttgttaaa gtccatgatg acggaagttg aatggagact gtcaaaatac | 1260 |
| gtgcctacag aaaaggaata catgattaat gcctctctta tcttcggcct aggtccaatc | 1320 |
| gttttaccag ctttgtattt cgttggtcca aagatttcag aaagtatagt aaaggaccca | 1380 |
| gaatatgatg aattgttcaa actaatgtca acatgtggta gattgttgaa tgacgtgcaa | 1440 |
| acgttcgaaa gagaatacaa tgagggtaaa ctgaattctg tcagtctatt ggttcttcac | 1500 |
| ggaggcccaa tgtctatttc agacgcaaag aggaaattac aaaagcctat tgatacgtgt | 1560 |
| agaagagatc ttctttcttt ggtccttaga gaagagtctg tagtaccaag accatgtaag | 1620 |
| gaactattct ggaaaatgtg taaagtgtgc tatttctttt actcaacaac tgatgggttt | 1680 |
| tctagtcaag tcgaaagagc aaaagaggta gacgctgtca taaatgagcc actgaagttg | 1740 |
| caaggttctc atacactggt atctgatgtt taa | 1773 |

SEQ ID NO: 48
Z. mays

| | |
|---|---|
| MAMPVKLTPA SLSLKAVCCR FSSGGHALRF GSSLPCWRRT PTQRSTSSST TRPAAEVSSG | 60 |
| KSKQHDQEAS EATIRQQLQL VDVLENMGIS RHFAAEIKCI LDRTYRSWLQ RHEEIMLDTM | 120 |
| TCAMAFRILR LNGYNVSSDE LYHVVEASGL HNSLGGYLND TRTLLELHKA STVSISEDES | 180 |
| ILDSIGSRSR TLLREQLESG GALRKPSLFK EVEHALDGPF YTTLDRLHHR WNIENFNIIE | 240 |
| QHMLETPYLS NQHTSRDILA LSIRDFSSSQ FTYQQELQHL ESWVKECRLD QLQFARQKLA | 300 |
| YFYLSAAGTM FSPELSDART LWAKNGVLTT IVDDFFDVAG SKEELENLVM LVEMWDEHHK | 360 |
| VEFYSEQVEI IFSSIYDSVN QLGEKASLVQ DRSITKHLVE IWLDLLKSMM TEVEWRLSKY | 420 |
| VPTEKEYMIN ASLIFGLGPI VLPALYFVGP KISESIVKDP EYDELFKLMS TCGRLLNDVQ | 480 |
| TFEREYNEGK LNSVSLLVLH GGPMSISDAK RKLQKPIDTC RRDLLSLVLR EESVVPRPCK | 540 |
| ELFWKMCKVC YFFYSTTDGF SSQVERAKEV DAVINEPLKL QGSHTLVSDV | 590 |

SEQ ID NO: 49
Artificial Sequence

| | |
|---|---|
| atgcagaact tccatggtac aaaggaaagg atcaaaaaga tgtttgacaa gattgaattg | 60 |
| tccgtttctt cttatgatac agcctgggtt gcaatggtcc catcccctga ttgcccagaa | 120 |
| acaccttgtt ttccagaatg tactaaatgg atcctagaaa atcagttggg tgatggtagt | 180 |
| tggtcacttc ctcatggcaa tccacttcta gttaaagatg cattatcttc cactcttgct | 240 |
| tgtattctgg ctcttaaaag atggggaatc ggtgaggaac agattaacaa aggactgaga | 300 |
| ttcatagaac tcaactctgc tagtgtaacc gataacgaac aacacaaacc aattggattt | 360 |
| gacattatct ttccaggtat gattgaatac gctatagact tagacctgaa tctaccacta | 420 |
| aaaccaactg acattaactc catgttgcat cgtagagccc ttgaattgac atcaggtgga | 480 |
| ggcaaaaatc tagaaggtag aagagcttac ttggcctacg tctctgaagg aatcggtaag | 540 |
| ctgcaagatt gggaaatggc tatgaaatac caacgtaaaa acggatctct gttcaatagt | 600 |
| ccatcaacaa ctgcagctgc attcatccat atacaagatg ctgaatgcct ccactatatt | 660 |
| cgttctcttc tccagaaatt tggaaacgca gtccctacaa tataccctct cgatatctat | 720 |
| gccagacttt caatggtaga tgccctggaa cgtcttggta ttgatagaca tttcagaaag | 780 |
| gagagaaagt tcgttctgga tgaaacatac agattttggt tgcaaggaga agaggagatt | 840 |
| ttctccgata acgcaacctg tgctttggcc ttcagaatat tgagacttaa tggttacgat | 900 |
| gtctctcttg aagatcactt ctctaactct ctgggcggtt acttaaagga ctcaggagca | 960 |
| gctttagaac tgtacagagc cctccaattg tcttacccag acgagtccct cctggaaaag | 1020 |
| caaaattcta gaacttctta cttcttaaaa caaggtttat ccaatgtctc cctctgtggt | 1080 |
| gacagattgc gtaaaaacat aattggagag gtgcatgatg ctttaaactt ttccgaccac | 1140 |
| gctaacttac aaagattagc tattcgtaga aggattaagc attacgctac tgacgataca | 1200 |
| aggattctaa aaacttccta cagatgctca acaatcggta accaagattt tctaaaactt | 1260 |
| gcagtggaag atttcaatat ctgtcaatca atacaaagag aggaattcaa gcatattgaa | 1320 |
| agatgggtcg ttgaaagacg tctagacaag ttaaagttcg ctagacaaaa agaggcctat | 1380 |
| tgctatttct cagccgcagc aacattgttt gcccctgaat tgtctgatgc tagaatgtct | 1440 |
| tgggccaaaa atggtgtatt gacaactgtg gttgatgatt tcttcgatgt cggaggctct | 1500 |
| gaagaggaat tagttaactt gatagaattg atcgagcgtt gggatgtgaa tggcagtgca | 1560 |
| gatttttgta gtgaggaagt tgagattatc tattctgcta tccactcaac tatctctgaa | 1620 |
| ataggtgata agtcatttgg ctggcaaggt agagatgtaa agtctcaagt tatcaagatc | 1680 |
| tggctggact tattgaaatc aatgttaact gaagctcaat ggtcttcaaa caagtctgtt | 1740 |
| cctaccctag atgagtatat gacaaccgcc catgtttcat tcgcacttgg tccaattgta | 1800 |
| cttccagcct tatacttcgt tggcccaaag ttgtcagaag aggttgcagg tcatcctgaa | 1860 |
| ctactaaacc tctacaaagt cacatctact tgtggcagac tactgaatga ttggagaagt | 1920 |
| tttaagagag aatccgagga aggtaagctc aacgctatta gtttatacat gatccactcc | 1980 |
| ggtggtgctt ctacagaaga ggaaacaatc gaacatttca aaggtttgat tgattctcag | 2040 |
| agaaggcaac tgttacaatt ggtgttgcaa gagaaggata gtatcatacc tagaccatgt | 2100 |
| aaagatctat tttggaatat gattaagtta ttacacactt tctacatgaa agatgatggc | 2160 |
| ttcacctcaa atgagatgag gaatgtagtt aaggcaatca ttaacgaacc aatctcactg | 2220 |
| gatgaattat ga | 2232 |

SEQ ID NO: 50
P. trichocarpa

| | |
|---|---|
| MSCIRPWFCP SSISATLTDP ASKLVTGEFK TTSLNFHGTK ERIKKMFDKI ELSVSSYDTA | 60 |
| WVAMVPSPDC PETPCFPECT KWILENQLGD GSWSLPHGNP LLVKDALSST LACILALKRW | 120 |
| GIGEEQINKG LRFIELNSAS VTDNEQHKPI GFDIIFPGMI EYAKDLDLNL PLKPTDINSM | 180 |
| LHRRALELTS GGGKNLEGRR AYLAYVSEGI GKLQDWEMAM KYQRKNGSLF NSPSTTAAAF | 240 |
| IHIQDAECLH YIRSLLQKFG NAVPTIYPLD IYARLSMVDA LERLGIDRHF RKERKFVLDE | 300 |
| TYRFWLQGEE EIFSDNATCA LAFRILRLNG YDVSLEDHFS NSLGGYLKDS GAALELYRAL | 360 |
| QLSYPDESLL EKQNSRTSYF LKQGLSNVSL CGDRLRKNII GEVHDALNFP DHANLQRLAI | 420 |
| RRRIKHYATD DTRILKTSYR CSTIGNQDFL KLAVEDFNIC QSIQREEFKH IERWVVERRL | 480 |
| DKLKFARQKE AYCYFSAAAT LFAPELSDAR MSWAKNGVLT TVVDDFFDVG GSEEELVNLI | 540 |
| ELIERWDVNG SADFCSEEVE IIYSAIHSTI SEIGDKSFGW QGRDVKSHVI KIWLDLLKSM | 600 |
| LTEAQWSSNK SVPTLDEYMT TAHVSFALGP IVLPALYFVG PKLSEEVAGH PELLNLYKVM | 660 |
| STCGRLLNDW RSFKRESEEG KLNAISLYMI HSGGASTEEE TIEHFKGLID SQRRQLLQLV | 720 |
| LQEKDSIIPR PCKDLFWNMI KLLHTFYMKD DGFTSNEMRN VVKAIINEPI SLDEL | 775 |

SEQ ID NO: 51
Artificial Sequence

| | |
|---|---|
| atgtctatca accttcgctc ctccggttgt tcgtctccga tctcagctac tttggaacga | 60 |
| ggattggact cagaagtaca gacaagagct aacaatgtga gctttgagca aacaaaggag | 120 |
| aagattagga agatgttgga gaaagtggag ctttctgttt cggcctacga tactagttgg | 180 |
| gtagcaatgg ttccatcacc gagctcccaa aatgctccac ttttcccaca gtgtgtgaaa | 240 |
| tggttattgg ataatcaaca tgaagatgga tcttggggac ttgataacca tgaccatcaa | 300 |
| tctcttaaga aggatgtgtt atcatctaca ctggctagta tcctcgcgtt aaagaagtgg | 360 |
| ggaattggtg aaagacaaat aaacaagggt ctccagttta ttgagctgaa ttctgcatta | 420 |
| gtcactgatg aaaccataca gaaaccaaca gggtttgata ttatatttcc tgggatgatt | 480 |
| aaatatgcta gagatttgaa tctgacgatt ccattgggct cagaagtggt ggatgacatg | 540 |
| atacgaaaaa gagatctgga tcttaaatgt gatagtgaaa agttttcaaa gggaagagaa | 600 |
| gcatatctgg cctatgtttt agaggggaca agaaacctaa aagattggga tttgatagtc | 660 |
| aaatatcaaa ggaaaaatgg gtcactgttt gattctccag ccacaacagc agctgctttt | 720 |
| actcagtttg ggaatgatgg ttgtctccgt tatctctgtt ctctccttca gaaattcgag | 780 |
| gctgcagttc cttcagttta tccatttgat caatatgcac gccttagtat aattgtcact | 840 |
| cttgaaagct taggaattga tagagatttc aaaaccgaaa tcaaaagcat attggatgaa | 900 |
| acctatagat attggcttcg tggggatgaa gaaatatgtt tggacttggc cacttgtgct | 960 |
| ttggctttcc gattattgct tgctcatggc tatgatgtgt cttacgatcc gctaaaacca | 1020 |
| tttgcagaag aatctggttt ctctgatact ttggaaggat atgttaagaa tacgttttct | 1080 |
| gtgttagaat tatttaaggc tgctcaaagt tatccacatg aatcagcttt gaagaagcag | 1140 |
| tgttgttgga ctaaacaata tctggagatg gaattgtcca gctgggttaa gacctctgtt | 1200 |
| cgagataaat acctcaagaa agaggtcgag gatgctcttg cttttccctc ctatgcaagc | 1260 |
| ctagaaagat cagatcacag gagaaaaata ctcaatggtt ctgctgtgga aaacaccaga | 1320 |
| gttacaaaaa cctcatatcg tttgcacaat atttgcacct ctgatatcct gaagttagct | 1380 |
| gtggatgact tcaatttctg ccagtccata caccgtgaag aaatggaacg tcttgatagg | 1440 |
| tggattgtgg agaatagatt gcaggaactg aaatttgcca gacagaagct ggcttactgt | 1500 |
| tatttctctg gggctgcaac tttattttct ccagaactat ctgatgctcg tatatcgtgg | 1560 |
| gccaaaggtg gagtacttac aacggttgta gacgacttct ttgatgttgg agggtccaaa | 1620 |
| gaagaactgg aaaacctcat acacttggtc gaaaagtggg atttgaacgg tgttcctgag | 1680 |
| tacagctcag aacatgttga gatcatattc tcagttctaa gggacaccat tctcgaaaca | 1740 |
| ggagacaaag cattcaccta tcaaggacgc aatgtgacac accacattgt gaaaatttgg | 1800 |
| ttggatctgc tcaagtctat gttgagagaa gccgagtggt ccagtgacaa gtcaacacca | 1860 |
| agcttggagg attacatgga aaatgcgtac atatcatttg cattaggacc aattgtcctc | 1920 |
| ccagctacct atctgatcgg acctccactt ccagagaaga cagtcgatag ccaccaatat | 1980 |
| aatcagctct acaagctcgt gagcactatg ggtcgtcttc taaatgacat acaaggtttt | 2040 |
| aagagagaaa gcgcggaagg gaagctgaat gcggtttcat tgcacatgaa acacgagaga | 2100 |
| gacaatcgca gcaaagaagt gatcatagaa tcgatgaaag gtttagcaga gagaaagagg | 2160 |
| gaagaattgc ataagctagt tttggaggag aaaggaagtg tggttccaag ggaatgcaaa | 2220 |
| gaagcgttct tgaaaatgag caaagtgttg aacttatttt acaggaagga cgatggattc | 2280 |
| acatcaaatg atctgatgag tcttgttaaa tcagtgatct acgagcctgt tagcttacag | 2340 |
| aaagaatctt taacttga | 2358 |

SEQ ID NO: 52
A. thaliana

| | |
|---|---|
| MSINLRSSGC SSPISATLER GLDSEVQTRA NNVSFEQTKE KIRKMLEKVE LSVSAYDTSW | 60 |
| VAMVPSPSSQ NAPLFPQCVK WLLDNQHEDG SWGLDNHDHQ SLKKDVLSST LASILALKKW | 120 |
| GIGERQINKG LQFIELNSAL VTDETIQKPT GFDIIFPGMI KYARDLNLTI PLGSEVVDDM | 180 |
| IRKRDLDLKC DSEKFSKGRE AYLAYVLEGT RNLKDWDLIV KYQRKNGSLF DSPATTAAAF | 240 |
| TQFGNDGCLR YLCSLLQKFE AAVPSVYPFD QYARLSIIVT LESLGIDRDF KTEIKSILDE | 300 |
| TYRYWLRGDE EICLDLATCA LAFRLLLAHG YDVSYDPLKP FAEESGFSDT LEGYVKNTFS | 360 |
| VLELFKAAQS YPHESALKKQ CCWTKQYLEM ELSSWVKTSV RDKYLKKEVE DALAFPSYAS | 420 |
| LERSDHRRKI LNGSAVENTR VTKTSYRLHN ICTSDILKLA VDDFNFCQSI HREEMERLDR | 480 |
| WIVENRLQEL KFARQKLAYC YFSGAATLFS PELSDARISW AKGGVLTTVV DDFFDVGGSK | 540 |
| EELENLIHLV EKWDLNGVPE YSSEHVEIIF SVLRDTILET GDKAFTYQGR NVTHHIVKIW | 600 |
| LDLLKSMLRE AEWSSDKSTP SLEDYMENAY ISFALGPIVL PATYLIGPPL PEKTVDSHQY | 660 |
| NQLYKLVSTM GRLLNDIQGF KRESAEGKLN AVSLHMKHER DNRSKEVIIE SMKGLAERKR | 720 |
| EELHKLVLEE KGSVVPRECK EAFLKMSKVL NLFYRKDDGF TSNDLMSLVK SVIYEPVSLQ | 780 |
| KESLT | 785 |

SEQ ID NO: 53
Artificial Sequence

| | |
|---|---|
| atggaatttg atgaaccatt ggttgacgaa gcaagatctt tagtgcagcg tactttacaa | 60 |
| gattatgatg acagatacgg cttcggtact atgtcatgtg ctgcttatga tacagcctgg | 120 |
| gtgtctttag ttacaaaaac agtcgatggg agaaaacaat ggcttttccc agagtgtttt | 180 |
| gaatttctac tagaaacaca atctgatgcc ggaggatggg aaatcgggaa ttcagcacca | 240 |
| atcgacggta tattgaatac agctgcatcc ttacttgctc taaaacgtca cgttcaaact | 300 |
| gagcaaatca tccaacctca acatgaccat aaggatctag caggtagagc tgaacgtgcc | 360 |
| gctgcatctt tgagagcaca attggctgca ttggatgtgt ctacaactga acacgtcggt | 420 |
| tttgagataa ttgttcctgc aatgctagac ccattagaag ccgaagatcc atctctagtt | 480 |
| ttcgattttc cagctaggaa acctttgatg aagattcatg atgctaagat gagtagattc | 540 |
| aggccagaat acttgtatgg caaacaacca atgaccgcct tacattcatt agaggctttc | 600 |
| ataggcaaaa tcgacttcga taaggtaaga caccaccgta cccatgggtc tatgatgggt | 660 |
| tctccttcat ctaccgcagc ctacttaatg cacgcttcac aatgggatgg tgactcagag | 720 |
| gcttacctta gacacgtgat taaacacgca gcagggcagg gaactggtgc tgtaccatct | 780 |
| gctttcccat caacacattt tgagtcatct tggattctta ccacattgtt tagagctgga | 840 |
| ttttcagctt ctcatcttgc ctgtgatgag ttgaacaagt tggtcgagat acttgagggc | 900 |
| tcattcgaga aggaaggtgg ggcaatcggt tacgctccag ggtttcaagc agatgttgat | 960 |
| gatactgcta aaacaataag tacattagca gtccttggaa gagatgctac accaagacaa | 1020 |
| atgatcaagg tatttgaagc taatacacat tttagaacat accctggtga aagagatcct | 1080 |
| tctttgacag ctaattgtaa tgctctatca gccttactac accaaccaga tgcagcaatg | 1140 |
| tatggatctc aaattcaaaa gattaccaaa tttgtctgtg actattggtg gaagtctgat | 1200 |
| ggtaagatta aagataagtg gaacacttgc tacttgtacc catctgtctt attagttgag | 1260 |
| gttttggttg atcttgttag tttattggag cagggtaaat tgcctgatgt tttggatcaa | 1320 |
| gagcttcaat acagagtcgc catcacattg ttccaagcat gtttaaggcc attactagac | 1380 |
| caagatgccg aaggatcatg gaacaagtct atcgaagcca cagcctacgg catccttatc | 1440 |
| ctaactgaag ctaggagagt ttgtttcttc gacagattgt ctgagccatt gaatgaggca | 1500 |
| atccgtagag gtatcgcttt cgccgactct atgtctggaa ctgaagctca gttgaactac | 1560 |
| atttggatcg aaaaggttag ttacgcacct gcattattga ctaaatccta tttgttagca | 1620 |
| gcaagatggg ctgctaagtc tcctttaggc gcttccgtag gctcttcttt gtggactcca | 1680 |
| ccaagagaag gattggataa gcatgtcaga ttattccatc aagctgagtt attcagatcc | 1740 |
| cttccagaat gggaattaag agcctccatg attgaagcag ctttgttcac accacttcta | 1800 |
| agagcacata gactagacgt tttccctaga caagatgtag gtgaagacaa atatcttgat | 1860 |
| gtagttccat tcttttggac tgccgctaac aacagagata gaacttacgc ttccactcta | 1920 |
| ttcctttacg atatgtgttt tatcgcaatg ttaaacttcc agttagacga attcatggag | 1980 |
| gccacagccg gtatcttatt cagagatcat atggatgatt tgaggcaatt gattcatgat | 2040 |
| cttttggcag agaaaacttc cccaaagagt tctggtagaa gtagtcaggg cacaaaagat | 2100 |
| gctgactcag gtatagagga agacgtgtca atgtccgatt cagcttcaga ttcccaggat | 2160 |
| agaagtccag aatacgactt ggttttcagt gcattgagta cctttacaaa acatgtcttg | 2220 |
| caacacccat ctatacaaag tgcctctgta tgggatagaa aactacttgc tagagagatg | 2280 |
| aaggcttact tacttgctca tatccaacaa gcagaagatt caactccatt gtctgaattg | 2340 |
| aaagatgtgc ctcaaaagac tgatgtaaca agagtttcta catctactac taccttcttt | 2400 |
| aactgggtta gaacaacttc cgcagaccat atatcctgcc catactcctt ccactttgta | 2460 |
| gcatgccatc taggcgcagc attgtcacct aaagggtcta acggtgattg ctatccttca | 2520 |
| gctggtgaga agttcttggc agctgcagtc tgcagacatt tggccaccat gtgtagaatg | 2580 |
| tacaacgatc ttggatcagc tgaacgtgat tctgatgaag gtaatttgaa ctccttggac | 2640 |
| ttccctgaat tcgccgattc cgcaggaaac ggagggatag aaattcagaa ggccgctcta | 2700 |
| ttaaggttag ctgagtttga gagagattca tacttagagg ccttccgtcg tttacaagat | 2760 |
| gaatccaata gagttcacgg tccagccggt ggtgatgaag ccagattgtc cagaaggaga | 2820 |
| atggcaatcc ttgaattctt cgcccagcag gtagatttgt acggtcaagt atacgtcatt | 2880 |
| agggatattt ccgctcgtat tcctaaaaac gaggttgaga aaaagagaaa attggatgat | 2940 |
| gctttcaatt ga | 2952 |
| SEQ ID NO: 54 | |
| P. amygdali | |
| MEFDEPLVDE ARSLVQRTLQ DYDDRYGFGT MSCAAYDTAW VSLVTKTVDG RKQWLFPECF | 60 |
| EFLLETQSDA GGWEIGNSAP IDGILNTAAS LLALKRHVQT EQIIQPQHDH KDLAGRAERA | 120 |
| AASLRAQLAA LDVSTTEHVG FEIIVPAMLD PLEAEDPSLV FDFPARKPLM KIHDAKMSRF | 180 |
| RPEYLYGKQP MTALHSLEAF IGKIDFDKVR HHRTHGSMMG SPSSTAAYLM HASQWDGDSE | 240 |
| AYLRHVIKHA AGQGTGAVPS AFPSTHFESS WILTTLFRAG FSASHLACDE LNKLVEILEG | 300 |
| SFEKEGGAIG YAPGFQADVD DTAKTISTLA VLGRDATPRQ MIKVFEANTH FRTYPGERDP | 360 |
| SLTANCNALS ALLHQPDAAM YGSQIQKITK FVCDYWWKSD GKIKDKWNTC YLYPSVLLVE | 420 |
| VLVDLVSLLE QGKLPDVLDQ ELQYRVAITL FQACLRPLLD QDAEGSWNKS IEATAYGILI | 480 |
| LTEARRVCFF DRLSEPLNEA IRRGIAFADS MSGTEAQLNY IWIEKVSYAP ALLTKSYLLA | 540 |
| ARWAAKSPLG ASVGSSLWTP PREGLDKHVR LFHQAELFRS LPEWELRASM IEAALFTPLL | 600 |
| RAHRLDVFPR QDVGEDKYLD VVPFFWTAAN NRDRTYASTL FLYDMCFIAM LNFQLDEFME | 660 |
| ATAGILFRDH MDDLRQLIHD LLAEKTSPKS SGRSSQGTKD ADSGIEEDVS MSDSASDSQD | 720 |
| RSPEYDLVFS ALSTFTKHVL QHPSIQSASV WDRKLLAREM KAYLLAHIQQ AEDSTPLSEL | 780 |
| KDVPQKTDVT RVSTSTTTFF NWVRTTSADH ISCPYSFHFV ACHLGAALSP KGSNGDCYPS | 840 |
| AGEKFLAAAV CRHLATMCRM YNDLGSAERD SDEGNLNSLD FPEFADSAGN GGIEIQKAAL | 900 |
| LRLAEFERDS YLEAFRRLQD ESNRVHGPAG GDEARLSRRR MAILEFFAQQ VDLYGQVYVI | 960 |
| RDISARIPKN EVEKKRKLDD AFN | 983 |

SEQ ID NO: 55
Artificial Sequence

| | |
|---|---|
| atggcttcta gtacacttat ccaaaacaga tcatgtggcg tcacatcatc tatgtcaagt | 60 |
| tttcaaatct tcagaggtca accactaaga tttcctggca ctagaacccc agctgcagtt | 120 |
| caatgcttga aaaagaggag atgccttagg ccaaccgaat ccgtactaga atcatctcct | 180 |
| ggctctggtt catatagaat agtaactggc ccttctggaa ttaaccctag ttctaacggg | 240 |
| cacttgcaag agggttcctt gactcacagg ttaccaatac caatggaaaa atctatcgat | 300 |
| aacttccaat ctactctata tgtgtcagat atttggtctg aaacactaca gagaactgaa | 360 |
| tgtttgctac aagtaactga aaacgtccag atgaatgagt ggattgagga aattagaatg | 420 |
| tactttagaa atatgacttt aggtgaaatt tccatgtccc cttacgacac tgcttgggtg | 480 |
| gctagagttc cagcgttgga cggttctcat gggcctcaat tccacagatc tttgcaatgg | 540 |
| attatcgaca accaattacc agatggggac tggggcgaac cttctctttt cttgggttac | 600 |
| gatagagttt gtaatacttt agcctgtgtg attgcgttga aaacatgggg tgttggggca | 660 |
| caaaacgttg aaagaggaat tcagttccta caatctaaca tatacaagat ggaggaagat | 720 |
| gacgctaatc atatgccaat aggattcgaa atcgtattcc ctgctatgat ggaagatgcc | 780 |
| aaagcattag gtttggattt gccatacgat gctactattt tgcaacagat ttcagccgaa | 840 |
| agagagaaaa agatgaaaaa gatcccaatg gcaatggtgt acaaataccc aaccacttta | 900 |
| cttcactcct tagaaggctt gcatagagaa gttgattgga ataagttgtt acaattacaa | 960 |
| tctgaaaatg gtagttttct ttattcacct gcttcaaccg catgcgcctt aatgtacact | 1020 |
| aaggacgtta aatgttttga ttacttaaac cagttgttga tcaagttcga ccacgcatgc | 1080 |
| ccaaatgtat atccagtcga tctattcgaa agattatgga tggttgacag attgcagaga | 1140 |
| ttagggatct ccagatactt tgaaagagag attagagatt gtttacaata cgtctacaga | 1200 |
| tattggaaag attgtggaat cggatgggct tctaactctt ccgtacaaga tgttgatgat | 1260 |
| acagccatgg cgtttagact tttaaggact catggtttcg acgtaaagga agattgcttt | 1320 |
| agacagtttt tcaaggacgg agaattcttc tgcttcgcag gccaatcatc tcaagcagtt | 1380 |
| acaggcatgt ttaatctttc aagagccagt caaacattgt ttccaggaga atctttattg | 1440 |
| aaaaaggcta gaaccttctc tagaaacttc ttgagaacaa agcatgagaa caacgaatgt | 1500 |
| ttcgataaat ggatcattac taaagatttg gctggtgaag tcgagtataa cttgaccttc | 1560 |
| ccatggtatg cctctttgcc tagattagaa cataggacat acttagatca atatggaatc | 1620 |
| gatgatatct ggataggcaa atctttatac aaaatgcctg ctgttaccaa cgaagttttc | 1680 |
| ctaaagttgg caaaggcaga ctttaacatg tgtcaagctc tacacaaaaa ggaattggaa | 1740 |
| caagtgataa agtggaacgc gtcctgtcaa ttcagagatc ttgaattcgc cagacaaaaa | 1800 |
| tcagtagaat gctattttgc tggtgcagcc acaatgttcg aaccagaaat ggttcaagct | 1860 |
| agattagtct gggcaagatg ttgtgtattg acaactgtct tagacgatta ctttgaccac | 1920 |
| gggacacctg ttgaggaact tagagtgttt gttcaagctg tcagaacatg gaatccagag | 1980 |
| ttgatcaacg gtttgccaga gcaagctaaa atcttgttta tgggcttata caaaacagtt | 2040 |
| aacacaattg cagaggaagc attcatggca cagaaaagag acgtccatca tcatttgaaa | 2100 |
| cactattggg acaagttgat aacaagtgcc ctaaaggagg ccgaatgggc agagtcaggt | 2160 |
| tacgtcccaa catttgatga atacatggaa gtagctgaaa tttctgttgc tctagaacca | 2220 |
| attgtctgta gtaccttgtt ctttgcgggt catagactag atgaggatgt tctagatagt | 2280 |
| tacgattacc atctagttat gcatttggta aacagagtcg gtagaatctt gaatgatata | 2340 |
| caaggcatga agagggaggc ttcacaaggt aagatctcat cagttcaaat ctacatggag | 2400 |
| gaacatccat ctgttccatc tgaggccatg gcgatcgctc atcttcaaga gttagttgat | 2460 |
| aattcaatgc agcaattgac atacgaagtt cttaggttca ctgcggttcc aaaaagttgt | 2520 |
| aagagaatcc acttgaatat ggctaaaatc atgcatgcct tctacaagga tactgatgga | 2580 |
| ttctcatccc ttactgcaat gacaggattc gtcaaaaagg ttcttttcga acctgtgcct | 2640 |
| gagtaa | 2646 |

SEQ ID NO: 56
P. patens

| | |
|---|---|
| MASSTLIQNR SCGVTSSMSS FQIFRGQPLR FPGTRTPAAV QCLKKRRCLR PTESVLESSP | 60 |
| GSGSYRIVTG PSGINPSSNG HLQEGSLTHR LPIPMEKSID NFQSTLYVSD IWSETLQRTE | 120 |
| CLLQVTENVQ MNEWIEEIRM YFRNMTLGEI SMSPYDTAWV ARVPALDGSH GPQFHRSLQW | 180 |
| IIDNQLPDGD WGEPSLFLGY DRVCNTLACV IALKTWGVGA QNVERGIQFL QSNIYKMEED | 240 |
| DANHMPIGFE IVFPAMMEDA KALGLDLPYD ATILQQISAE REKKMKKIPM AMVYKYPTTL | 300 |
| LHSLEGLHRE VDWNKLLQLQ SENGSFLYSP ASTACALMYT KDVKCFDYLN QLLIKFDHAC | 360 |
| PNVYPVDLFE RLWMVDRLQR LGISRYFERE IRDCLQYVYR YWKDCGIGWA SNSSVQDVDD | 420 |
| TAMAFRLLRT HGFDVKEDCF RQFFKDGEFF CFAGQSSQAV TGMFNLSRAS QTLFPGESLL | 480 |
| KKARTFSRNF LRTKHENNEC FDKWIITKDL AGEVEYNLTF PWYASLPRLE HRTYLDQYGI | 540 |
| DDIWIGKSLY KMPAVTNEVF LKLAKADFNM CQALHKKELE QVIKWNASCQ FRDLEFARQK | 600 |
| SVECYFAGAA TMFEPEMVQA RLVWARCCVL TTVLDDYFDH GTPVEELRVF VQAVRTWNPE | 660 |
| LINGLPEQAK ILFMGLYKTV NTIAEEAFMA QKRDVHHHLK HYWDKLITSA LKEAEWAESG | 720 |
| YVPTFDEYME VAEISVALEP IVCSTLFFAG HRLDEDVLDS YDYHLVMHLV NRVGRILNDI | 780 |
| QGMKREASQG KISSVQIYME EHPSVPSEAM AIAHLQELVD NSMQQLTYEV LRFTAVPKSC | 840 |
| KRIHLNMAKI MHAFYKDTDG FSSLTAMTGF VKKVLFEPVP E | 881 |

SEQ ID NO: 57
Artificial Sequence

| | |
|---|---|
| atgcctggta aaattgaaaa tggtacccca aaggacctca agactggaaa tgattttgtt | 60 |
| tctgctgcta agagtttact agatcgagct ttcaaaagtc atcattccta ctacggatta | 120 |
| tgctcaactt catgtcaagt ttatgataca gcttgggttg caatgattcc aaaaacaaga | 180 |
| gataatgtaa aacagtggtt gtttccagaa tgtttccatt acctcttaaa aacacaagcc | 240 |
| gcagatggct catggggttc attgcctaca acacagacag cgggtatcct agatacagcc | 300 |
| tcagctgtgc tggcattatt gtgccacgca caagagcctt tacaaatatt ggatgtatct | 360 |
| ccagatgaaa tggggttgag aatagaacac ggtgtcacat ccttgaaacg tcaattagca | 420 |
| gtttggaatg atgtggagga caccaaccat attggcgtcg agtttatcat accagcctta | 480 |
| ctttccatgc tagaaaagga attagatgtt ccatcttttg aatttccatg taggtccatc | 540 |
| ttagagagaa tgcacgggga gaaattaggt catttcgacc tggaacaagt ttacggcaag | 600 |
| ccaagctcat tgttgcactc attggaagca tttctcggta agctagattt tgatcgacta | 660 |
| tcacatcacc tataccacgg cagtatgatg gcatctccat cttcaacggc tgcttatctt | 720 |
| attggggcta caaaatggga tgacgaagcc gaagattacc taagacatgt aatgcgtaat | 780 |
| ggtgcaggac atgggaatgg aggtatttct ggtacatttc caactactca tttcgaatgt | 840 |
| agctggatta tagcaacgtt gttaaaggtt ggctttactt tgaagcaaat tgacggcgat | 900 |
| ggcttaagag gtttatcaac catcttactt gaggcgcttc gtgatgagaa tggtgtcata | 960 |
| ggctttgccc ctagaacagc agatgtagat gacacagcca aagctctatt ggccttgtca | 1020 |
| ttggtaaacc agccagtgtc acctgatatc atgattaagg tctttgaggg caaagaccat | 1080 |
| tttaccactt ttggttcaga aagagatcca tcattgactt ccaacctgca cgtcctttta | 1140 |
| tctttactta aacaatctaa cttgtctcaa taccatcctc aaatcctcaa aacaacatta | 1200 |
| ttcacttgta gatggtggtg gggttccgat cattgtgtca aagacaaatg gaatttgagt | 1260 |
| cacctatatc caactatgtt gttggttgaa gccttcactg aagtgctcca tctcattgac | 1320 |
| ggtggtgaat tgtctagtct gtttgatgaa tcctttaagt gtaagattgg tcttagcatc | 1380 |
| tttcaagcgg tacttagaat aatcctcacc caagacaacg acggctcttg gagaggatac | 1440 |
| agagaacaga cgtgttacgc aatattggct ttagttcaag cgagacatgt atgctttttc | 1500 |
| actcacatgg ttgacagact gcaatcatgt gttgatcgag gtttctcatg gttgaaatct | 1560 |
| tgctcttttc attctcaaga cctgacttgg acctctaaaa cagcttatga agtgggtttc | 1620 |
| gtagctgaag catataaact agctgcttta caatctgctt ccctggaggt tcctgctgcc | 1680 |
| accattggac attctgtcac gtctgccgtt ccatcaagtg atcttgaaaa atacatgaga | 1740 |
| ttggtgagaa aaactgcgtt attctctcca ctggatgagt ggggtctaat ggcttctatc | 1800 |
| atcgaatctt catttttcgt accattactg caggcacaaa gagttgaaat ataccctaga | 1860 |
| gataatatca aggtggacga agataagtac ttgtctatta tcccattcac atgggtcgga | 1920 |
| tgcaataata ggtctagaac tttcgcaagt aacagatggc tatacgatat gatgtacctt | 1980 |
| tcattactcg gctatcaaac cgacgagtac atggaagctg tagctgggcc agtgtttggg | 2040 |
| gatgtttcct tgttacatca aacaattgat aaggtgattg ataatacaat gggtaacctt | 2100 |
| gcgagagcca atggaacagt acacagtggt aatggacatc agcacgaatc tcctaatata | 2160 |
| ggtcaagtcg aggacacctt gactcgtttc acaaattcag tcttgaatca caaagacgtc | 2220 |
| cttaactcta gctcatctga tcaagatact ttgagaagag agtttagaac attcatgcac | 2280 |
| gctcatataa cacaaatcga agataactca cgattcagta agcaagcctc atccgatgcg | 2340 |
| ttttcctctc ctgaacaatc ttactttcaa tgggtgaact caactggtgg ctcacatgtc | 2400 |
| gcttgcgcct attcatttgc cttctctaat tgcctcatgt ctgcaaattt gttgcagggt | 2460 |
| aaagacgcat ttccaagcgg aacgcaaaag tacttaatct cctctgttat gagacatgcc | 2520 |
| acaaacatgt gtagaatgta taacgacttt ggctctattg ccagagacaa cgctgagaga | 2580 |
| aatgttaata gtattcattt tcctgagttt actctctgta acggaacttc tcaaaaccta | 2640 |
| gatgaaagga aggaaagact tctgaaaatc gcaacttacg aacaagggta tttggataga | 2700 |
| gcactagagg ccttggaaag acagagtaga gatgatgccg gagacagagc tggatctaaa | 2760 |
| gatatgagaa agttgaaaat cgttaagtta ttctgtgatg ttacggactt atacgatcag | 2820 |
| ctctacgtta tcaaagattt gtcatcctct atgaagtaa | 2859 |

SEQ ID NO: 58
G. fujikuroi

| | |
|---|---|
| MPGKIENGTP KDLKTGNDFV SAAKSLLDRA FKSHHSYYGL CSTSCQVYDT AWVAMIPKTR | 60 |
| DNVKQWLFPE CFHYLLKTQA ADGSWGSLPT TQTAGILDTA SAVLALLCHA QEPLQILDVS | 120 |
| PDEMGLRIEH GVTSLKRQLA VWNDVEDTNH IGVEFIIPAL LSMLEKELDV PSFEFPCRSI | 180 |
| LERMHGEKLG HFDLEQVYGK PSSLLHSLEA FLGKLDFDRL SHHLYHGSMM ASPSSTAAYL | 240 |
| IGATKWDDEA EDYLRHVMRN GAGHGNGGIS GTFPTTHFEC SWIIATLLKV GFTLKQIDGD | 300 |
| GLRGLSTILL EALRDENGVI GFAPRTADVD DTAKALLALS LVNQPVSPDI MIKVFEGKDH | 360 |
| FTTFGSERDP SLTSNLHVLL SLLKQSNLSQ YHPQILKTTL FTCRWWWGSD HCVKDKWNLS | 420 |
| HLYPTMLLVE AFTEVLHLID GGELSSLFDE SFKCKIGLSI FQAVLRIILT QDNDGSWRGY | 480 |
| REQTCYAILA LVQARHVCFF THMVDRLQSC VDRGFSWLKS CSFHSQDLTW TSKTAYEVGF | 540 |
| VAEAYKLAAL QSASLEVPAA TIGHSVTSAV PSSDLEKYMR LVRKTALFSP LDEWGLMASI | 600 |
| IESSFFVPLL QAQRVEIYPR DNIKVDEDKY LSIIPFTWVG CNNRSRTFAS NRWLYDMMYL | 660 |
| SLLGYQTDEY MEAVAGPVFG DVSLLHQTID KVIDNTMGNL ARANGTVHSG NGHQHESPNI | 720 |
| GQVEDTLTRF TNSVLNHKDV LNSSSSDQDT LRREFRTFMH AHITQIEDNS RFSKQASSDA | 780 |
| FSSPEQSYFQ WVNSTGGSHV ACAYSFAFSN CLMSANLLQG KDAFPSGTQK YLISSVMRHA | 840 |
| TNMCRMYNDF GSIARDNAER NVNSIHFPEF TLCNGTSQNL DERKERLLKI ATYEQGYLDR | 900 |
| ALEALERQSR DDAGDRAGSK DMRKLKIVKL FCDVTDLYDQ LYVIKDLSSS MK | 952 |

SEQ ID NO: 59
Artificial Sequence

| | |
|---|---|
| atggatgctg tgacgggttt gttaactgtc ccagcaaccg ctataactat tggtggaact | 60 |
| gctgtagcat tggcggtagc gctaatcttt tggtacctga aatcctacac atcagctaga | 120 |
| agatcccaat caaatcatct tccaagagtg cctgaagtcc caggtgttcc attgttagga | 180 |
| aatctgttac aattgaagga gaaaaagcca tacatgactt ttacgagatg ggcagcgaca | 240 |
| tatggaccta tctatagtat caaaactggg gctacaagta tggttgtggt atcatctaat | 300 |
| gagatagcca aggaggcatt ggtgaccaga ttccaatcca tatctacaag gaacttatct | 360 |
| aaagccctga aagtacttac agcagataag acaatggtcg caatgtcaga ttatgatgat | 420 |
| tatcataaaa cagttaagag acacatactg accgccgtct tgggtcctaa tgcacagaaa | 480 |
| aagcatagaa ttcacagaga tatcatgatg gataacatat ctactcaact tcatgaattc | 540 |
| gtgaaaaaca acccagaaca ggaagaggta gaccttagaa aaatctttca atctgagtta | 600 |
| ttcggcttag ctatgagaca agccttagga aaggatgttg aaagtttgta cgttgaagac | 660 |
| ctgaaaatca ctatgaatag agacgaaatc tttcaagtcc ttgttgttga tccaatgatg | 720 |
| ggagcaatcg atgttgattg gagagacttc tttccatacc taaagtgggt cccaaacaaa | 780 |
| aagttcgaaa atactattca acaaatgtac atcagaagag aagctgttat gaaatcttta | 840 |
| atcaaagagc acaaaaagag aatagcgtca ggcgaaaagc taaatagtta tatcgattac | 900 |
| cttttatctg aagctcaaac tttaaccgat cagcaactat tgatgtcctt gtgggaacca | 960 |
| atcattgaat cttcagatac aacaatggtc acaacagaat gggcaatgta cgaattagct | 1020 |
| aaaaacccta aattgcaaga taggttgtac agagacatta agtccgtctg tggatctgaa | 1080 |
| aagataaccg aagagcatct atcacagctg ccttacatta cagctatttt ccacgaaaca | 1140 |
| ctgagaagac actcaccagt tcctatcatt cctctaagac atgtacatga agataccgtt | 1200 |
| ctaggcggct accatgttcc tgctggcaca gaacttgccg ttaacatcta cggttgcaac | 1260 |
| atggacaaaa acgtttggga aaatccagag gaatggaacc cagaaagatt catgaaagag | 1320 |
| aatgagacaa ttgattttca aaagacgatg gccttcggtg gtggtaagag agtttgtgct | 1380 |
| ggttccttgc aagccctttt aactgcatct attgggattg ggagaatggt tcaagagttc | 1440 |
| gaatggaaac tgaaggatat gactcaagag gaagtgaaca cgataggcct aactacacaa | 1500 |
| atgttaagac cattgagagc tattatcaaa cctaggatct aa | 1542 |

SEQ ID NO: 60
S. rebaudiana

| | |
|---|---|
| MDAVTGLLTV PATAITIGGT AVALAVALIF WYLKSYTSAR RSQSNHLPRV PEVPGVPLLG | 60 |
| NLLQLKEKKP YMTFTRWAAT YGPIYSIKTG ATSMVVVSSN EIAKEALVTR FQSISTRNLS | 120 |
| KALKVLTADK TMVAMSDYDD YHKTVKRHIL TAVLGPNAQK KHRIHRDIMM DNISTQLHEF | 180 |
| VKNNPEQEEV DLRKIFQSEL FGLAMRQALG KDVESLYVED LKITMNRDEI FQVLVVDPMM | 240 |
| GAIDVDWRDF FPYLKWVPNK KFENTIQQMY IRREAVMKSL IKEHKKRIAS GEKLNSYIDY | 300 |
| LLSEAQTLTD QQLLMSLWEP IIESSDTTMV TTEWAMYELA KNPKLQDRLY RDIKSVCGSE | 360 |
| KITEEHLSQL PYITAIFHET LRRHSPVPII PLRHVHEDTV LGGYHVPAGT ELAVNIYGCN | 420 |
| MDKNVWENPE EWNPERFMKE NETIDFQKTM AFGGGKRVCA GSLQALLTAS IGIGRMVQEF | 480 |
| EWKLKDMTQE EVNTIGLTTQ MLRPLRAIIK PRI | 513 |

SEQ ID NO: 61
Artificial Sequence

| | |
|---|---|
| aagcttacta gtaaaatgga cggtgtcatc gatatgcaaa ccattccatt gagaaccgct | 60 |
| attgctattg gtggtactgc tgttgctttg gttgttgcat tatacttttg gttcttgaga | 120 |
| tcctacgctt ccccatctca tcattctaat catttgccac cagtacctga agttccaggt | 180 |
| gttccagttt tgggtaattt gttgcaattg aaagaaaaaa agccttacat gaccttcacc | 240 |
| aagtgggctg aaatgtatgg tccaatctac tctattagaa ctggtgctac ttccatggtt | 300 |
| gttgtctctt ctaacgaaat cgccaaagaa gttgttgtta ccagattccc atctatctct | 360 |
| accagaaaat tgtcttacgc cttgaaggtt ttgaccgaag ataagtctat ggttgccatg | 420 |
| tctgattatc acgattacca taagaccgtc aagagacata ttttgactgc tgttttgggt | 480 |
| ccaaacgccc aaaaaaagtt tagagcacat agagacacca tgatggaaaa cgtttccaat | 540 |
| gaattgcatg ccttcttcga aaagaaccca aatcaagaag tcaacttgag aaagatcttc | 600 |
| caatcccaat tattcggttt ggctatgaag caagccttgg gtaaagatgt tgaatccatc | 660 |
| tacgttaagg atttggaaac caccatgaag agagaagaaa tcttcgaagt tttggttgtc | 720 |
| gatccaatga tgggtgctat tgaagttgat tggagagact ttttcccata cttgaaatgg | 780 |
| gttccaaaca agtccttcga aaacatcatc catagaatgt acactagaag agaagctgtt | 840 |
| atgaaggcct tgatccaaga acacaagaaa agaattgcct ccggtgaaaa cttgaactcc | 900 |
| tacattgatt acttgttgtc tgaagcccaa accttgaccg ataagcaatt attgatgtct | 960 |
| ttgtgggaac ctattatcga atcttctgat accactatgg ttactactga atgggctatg | 1020 |
| tacgaattgg ctaagaatcc aaacatgcaa gacagattat acgaagaaat ccaatccgtt | 1080 |
| tgcggttccg aaaagattac tgaagaaaac ttgtcccaat tgccatactt gtacgctgtt | 1140 |
| ttccaagaaa ctttgagaaa gcactgtcca gttcctatta tgccattgag atatgttcac | 1200 |
| gaaaacaccg ttttgggtgg ttatcatgtt ccagctggta ctgaagttgc tattaacatc | 1260 |
| tacggttgca acatggataa gaaggtctgg gaaaatccag aagaatggaa tccagaaaga | 1320 |
| ttcttgtccg aaaaagaatc catggacttg tacaaaacta tggcttttgg tggtggtaaa | 1380 |
| agagtttgcg ctggttcttt acaagccatg gttatttctt gcattggtat cggtagattg | 1440 |
| gtccaagatt ttgaatggaa gttgaaggat gatgccgaag aagatgttaa cactttgggt | 1500 |
| ttgactaccc aaaagttgca tccattattg gccttgatta acccaagaaa gtaactcgag | 1560 |
| ccgcgg | 1566 |

SEQ ID NO: 62
L. sativa

| | |
|---|---|
| MDGVIDMQTI PLRTAIAIGG TAVALVVALY FWFLRSYASP SHHSNHLPPV PEVPGVPVLG | 60 |
| NLLQLKEKKP YMTFTKWAEM YGPIYSIRTG ATSMVVVSSN EIAKEVVVTR FPSISTRKLS | 120 |
| YALKVLTEDK SMVAMSDYHD YHKTVKRHIL TAVLGPNAQK KFRAHRDTMM ENVSNELHAF | 180 |
| FEKNPNQEVN LRKIFQSQLF GLAMKQALGK DVESIYVKDL ETTMKREEIF EVLVVDPMMG | 240 |
| AIEVDWRDFF PYLKWVPNKS FENIIHRMYT RREAVMKALI QEHKKRIASG ENLNSYIDYL | 300 |
| LSEAQTLTDK QLLMSLWEPI IESSDTTMVT TEWAMYELAK NPNMQDRLYE EIQSVCGSEK | 360 |
| ITEENLSQLP YLYAVFQETL RKHCPVPIMP LRYVHENTVL GGYHVPAGTE VAINIYGCNM | 420 |
| DKKVWENPEE WNPERFLSEK ESMDLYKTMA FGGGKRVCAG SLQAMVISCI GIGRLVQDFE | 480 |
| WKLKDDAEED VNTLGLTTQK LHPLLALINP RK | 512 |

SEQ ID NO: 63
R. suavissimus

| | |
|---|---|
| atggccaccc tccttgagca tttccaagct atgccctttg ccatccctat tgcactggct | 60 |
| gctctgtctt ggctgttcct cttttacatc aaagtttcat tcttttccaa caagagtgct | 120 |
| caggctaagc tccctcctgt gccagtggtt cctgggctgc cggtgattgg gaatttactg | 180 |
| caactcaagg agaagaaacc ctaccagact tttacaaggt gggctgagga gtatggacca | 240 |
| atctattcta tcaggactgg tgcttccacc atggtcgttc tcaataccac ccaagttgca | 300 |
| aaagaggcca tggtgaccag atatttatcc atctcaacca gaaagctatc aaacgcacta | 360 |
| aagattctta ctgctgataa atgtatggtt gcaataagtg actacaacga ttttcacaag | 420 |
| atgataaagc gatacatact ctcaaatgtt cttggaccta gtgctcagaa gcgtcaccgg | 480 |
| agcaacagag ataccttgag agctaatgtc tgcagccgat tgcattctca agtaaagaac | 540 |
| tctcctcgag aagctgtgaa tttcagaaga gtttttgagt gggaactctt tggaattgca | 600 |
| ttgaagcaag cctttggaaa ggacatagaa aagcccattt atgtggagga acttggcact | 660 |
| acactgtcaa gagatgagat ctttaaggtt ctagtgcttg acataatgga gggtgcaatt | 720 |
| gaggttgatt ggagagattt cttcccttac ctgagatgga ttccgaatac gcgcatggaa | 780 |
| acaaaaattc agcgactcta tttccgcagg aaagcagtga tgactgccct gatcaacgag | 840 |
| cagaagaagc gaattgcttc aggagaggaa atcaactgtt atatcgactt cttgcttaag | 900 |
| gaagggaaga cactgacaat ggaccaaata agtatgttgc tttgggagac ggttattgaa | 960 |
| acagcagata ctacaatggt aacgacagaa tgggctatgt atgaagttgc taaagactca | 1020 |
| aagcgtcagg atcgtctcta tcaggaaatc caaaaggttt gtggatcgga gatggttaca | 1080 |
| gaggaatact tgtcccaact gccgtacctg aatgcagttt tccatgaaac gctaaggaag | 1140 |
| cacagtccgg ctgcgttagt tcctttaaga tatgcacatg aagataccca actaggaggt | 1200 |
| tactacattc cagctggaac tgagattgct ataaacatat acgggtgtaa catggacaag | 1260 |
| catcaatggg aaagccctga ggaatggaaa ccggagagat ttttggaccc gaaatttgat | 1320 |
| cctatggatt tgtacaagac catggctttt ggggctggaa agagggtatg tgctggttct | 1380 |
| cttcaggcaa tgttaatagc gtgcccgacg attggtaggc tggtgcagga gtttgagtgg | 1440 |
| aagctgagag atggagaaga agaaaatgta gatactgttg ggctcaccac tcacaaacgc | 1500 |
| tatccaatgc atgcaatcct gaagccaaga agtta | 1535 |

SEQ ID NO: 64
Artificial Sequence

| | |
|---|---|
| atggctacct tgttggaaca ttttcaagct atgccattcg ctattccaat tgctttggct | 60 |
| gctttgtctt ggttgttttt gttctacatc aaggtttctt tcttctccaa caaatccgct | 120 |
| caagctaaat tgccaccagt tccagttgtt ccaggtttgc cagttattgg taatttgttg | 180 |
| caattgaaag aaaagaagcc ataccaaacc ttcactagat gggctgaaga atatggtcca | 240 |
| atctactcta ttagaactgg tgcttctact atggttgtct tgaacactac tcaagttgcc | 300 |
| aaagaagcta tggttaccag atacttgtct atctctacca gaaagttgtc caacgccttg | 360 |
| aaaattttga ccgctgataa gtgcatggtt gccatttctg attacaacga tttccacaag | 420 |
| atgatcaaga gatatatctt gtctaacgtt ttgggtccat ctgcccaaaa aagacataga | 480 |
| tctaacagag ataccttgag agccaacgtt tgttctagat tgcattccca agttaagaac | 540 |
| tctccaagag aagctgtcaa ctttagaaga gttttcgaat gggaattatt cggtatcgct | 600 |
| ttgaaacaag ccttcggtaa ggatattgaa aagccaatct acgtcgaaga attgggtact | 660 |
| actttgtcca gagatgaaat cttcaaggtt ttggtcttgg acattatgga aggtgccatt | 720 |
| gaagttgatt ggagagattt tttcccatac ttgcgttgga ttccaaacac cagaatggaa | 780 |
| actaagatcc aaagattata ctttagaaga aaggccgtta tgaccgcctt gattaacgaa | 840 |
| caaaagaaaa gaattgcctc cggtgaagaa atcaactgct acatcgattt cttgttgaaa | 900 |
| gaaggtaaga ccttgaccat ggaccaaatc tctatgttgt tgtgggaaac cgttattgaa | 960 |
| actgctgata ccacaatggt tactactgaa tgggctatgt acgaagttgc taaggattct | 1020 |
| aaaagacaag acagattata ccaagaaatc caaaaggtct gcggttctga aatggttaca | 1080 |
| gaagaatact tgtcccaatt gccatacttg aatgctgttt tccacgaaac tttgagaaaa | 1140 |
| cattctccag ctgctttggt tccattgaga tatgctcatg aagatactca attgggtggt | 1200 |
| tattacattc cagccggtac tgaaattgcc attaacatct acggttgcaa catggacaaa | 1260 |
| caccaatggg aatctccaga agaatggaag ccagaaagat ttttggatcc taagtttgac | 1320 |
| ccaatggact tgtacaaaac tatggctttt ggtgctggta aaagagtttg cgctggttct | 1380 |
| ttacaagcta tgttgattgc ttgtccaacc atcggtagat tggttcaaga atttgaatgg | 1440 |
| aagttgagag atggtgaaga agaaaacgtt gatactgttg gtttgaccac ccataagaga | 1500 |
| tatccaatgc atgctatttt gaagccaaga tcttaa | 1536 |

SEQ ID NO: 65
Artificial Sequence

| | |
|---|---|
| aagcttacta gtaaaatggc ctccatcacc catttcttac aagattttca agctactcca | 60 |
| ttcgctactg cttttgctgt tggtggtgtt tctttgttga tattcttctt cttcatccgt | 120 |
| ggtttccact ctactaagaa aaacgaatat tacaagttgc caccagttcc agttgttcca | 180 |
| ggtttgccag ttgttggtaa tttgttgcaa ttgaaagaaa agaagccata caagactttc | 240 |
| ttgagatggg ctgaaattca tggtccaatc tactctatta gaactggtgc ttctaccatg | 300 |
| gttgttgtta actctactca tgttgccaaa gaagctatgg ttaccagatt ctcttcaatc | 360 |
| tctaccagaa agttgtccaa ggctttggaa ttattgacct ccaacaaatc tatggttgcc | 420 |
| acctctgatt acaacgaatt tcacaagatg gtcaagaagt acatcttggc cgaattattg | 480 |
| ggtgctaatg ctcaaaagag acacagaatt catagagaca ccttgatcga aaacgtcttg | 540 |
| aacaaattgc atgcccatac caagaattct ccattgcaag ctgttaactt cagaaagatc | 600 |
| ttcgaatctg aattattcgg tttggctatg aagcaagcct tgggttatga tgttgattcc | 660 |
| ttgttcgttg aagaattggg tactaccttg tccagagaag aaatctacaa cgttttggtc | 720 |
| agtgacatgt tgaagggtgc tattgaagtt gattggagag actttttccc atacttgaaa | 780 |
| tggatcccaa acaagtcctt cgaaatgaag attcaaagat tggcctctag aagacaagcc | 840 |
| gttatgaact ctattgtcaa agaacaaaag aagtccattg cctctggtaa gggtgaaaac | 900 |
| tgttacttga attacttgtt gtccgaagct aagactttga ccgaaaagca aatttccatt | 960 |
| ttggcctggg aaaccattat tgaaactgct gatacaactg ttgttaccac tgaatgggct | 1020 |
| atgtacgaat tggctaaaaa cccaaagcaa caagacagat tatacaacga aatccaaaac | 1080 |
| gtctgcggta ctgataagat taccgaagaa catttgtcca agttgcctta cttgtctgct | 1140 |
| gtttttcacg aaaccttgag aaagtattct ccatctccat tggttccatt gagatacgct | 1200 |
| catgaagata ctcaattggg tggttattat gttccagccg gtactgaaat tgctgttaat | 1260 |
| atctacggtt gcaacatgga caagaatcaa tgggaaactc cagaagaatg gaagccagaa | 1320 |
| agatttttgg acgaaaagta cgatccaatg gacatgtaca agactatgtc ttttggttcc | 1380 |
| ggtaaaagag tttgcgctgg ttctttacaa gctagtttga ttgcttgtac ctccatcggt | 1440 |
| agattggttc aagaatttga atggagattg aaagacggtg aagttgaaaa cgttgatacc | 1500 |
| ttgggtttga ctacccataa gttgtatcca atgcaagcta tcttgcaacc tagaaactga | 1560 |
| ctcgagccgc gg | 1572 |

SEQ ID NO: 66
C. mollissima

| | |
|---|---|
| MASITHFLQD FQATPFATAF AVGGVSLLIF FFFIRGFHST KKNEYYKLPP VPVVPGLPVV | 60 |
| GNLLQLKEKK PYKTFLRWAE IHGPIYSIRT GASTMVVVNS THVAKEAMVT RFSSISTRKL | 120 |
| SKALELLTSN KSMVATSDYN EFHKMVKKYI LAELLGANAQ KRHRIHRDTL IENVLNKLHA | 180 |
| HTKNSPLQAV NFRKIFESEL FGLAMKQALG YDVDSLFVEE LGTTLSREEI YNVLVSDMLK | 240 |
| GAIEVDWRDF FPYLKWIPNK SFEMKIQRLA SRRQAVMNSI VKEQKKSIAS GKGENCYLNY | 300 |
| LLSEAKTLTE KQISILAWET IIETADTTVV TTEWAMYELA KNPKQQDRLY NEIQNVCGTD | 360 |
| KITEEHLSKL PYLSAVFHET LRKYSPSPLV PLRYAHEDTQ LGGYYVPAGT EIAVNIYGCN | 420 |
| MDKNQWETPE EWKPERFLDE KYDPMDMYKT MSFGSGKRVC AGSLQASLIA CTSIGRLVQE | 480 |
| FEWRLKDGEV ENVDTLGLTT HKLYPMQAIL QPRN | 514 |

SEQ ID NO: 67
Artificial Sequence

| | |
|---|---|
| atgatttcct tgttgttggg ttttgttgtc tcctccttct tgtttatctt cttcttgaaa | 60 |
| aaattgttgt tcttcttcag tcgtcacaaa atgtccgaag tttctagatt gccatctgtt | 120 |
| ccagttccag gttttccatt gattggtaac ttgttgcaat tgaaagaaaa gaagccacac | 180 |
| aagactttca ccaagtggtc tgaattatat ggtccaatct actctatcaa gatgggttcc | 240 |
| tcttctttga tcgtcttgaa ctctattgaa accgccaaag aagctatggt cagtagattc | 300 |
| tcttcaatct ctaccagaaa gttgtctaac gctttgactg ttttgacctg caacaaatct | 360 |
| atggttgcta cctctgatta cgatgacttt cataagttcg tcaagagatg cttgttgaac | 420 |
| ggtttgttgg gtgctaatgc tcaagaaaga aaaagacatt acagagatgc cttgatcgaa | 480 |
| aacgttacct ctaaattgca tgcccatacc agaaatcatc cacaagaacc agttaacttc | 540 |
| agagccattt tcgaacacga attattcggt gttgctttga aacaagcctt cggtaaagat | 600 |
| gtcgaatcca tctatgtaaa agaattgggt gtcaccttgt ccagagatga aattttcaag | 660 |
| gttttggtcc acgacatgat ggaaggtgct attgatgttg attggagaga tttcttccca | 720 |
| tacttgaaat ggatcccaaa caactctttc gaagccagaa ttcaacaaaa gcacaagaga | 780 |
| agattggctg ttatgaacgc cttgatccaa gacagattga atcaaaacga ttccgaatcc | 840 |
| gatgatgact gctacttgaa tttcttgatg tctgaagcta agaccttgac catggaacaa | 900 |
| attgctattt tggtttggga aaccattatc gaaactgctg ataccacttt ggttactact | 960 |
| gaatgggcta tgtacgaatt ggccaaacat caatctgttc aagatagatt attcaaagaa | 1020 |
| atccaatccg tctgcggtgg tgaaaagatc aaagaagaac aattgccaag attgccttac | 1080 |
| gtcaatggtg tttttcacga aaccttgaga aagtattctc cagctccatt ggttccaatt | 1140 |
| agatacgctc atgaagatac ccaaattggt ggttatcata ttccagccgg ttctgaaatt | 1200 |
| gccattaaca tctacggttg caacatggat aagaagagat gggaaagacc tgaagaatgg | 1260 |
| tggccagaaa gatttttgga agatagatac gaatcctccg acttgcataa gactatggct | 1320 |
| tttggtgctg gtaaaagagt ttgtgctggt gctttacaag ctagtttgat ggctggtatt | 1380 |
| gctatcggta gattggttca agaattcgaa tggaagttga gagatggtga agaagaaaac | 1440 |
| gttgatactt acggtttgac ctcccaaaag ttgtatccat tgatggccat tatcaaccca | 1500 |
| agaagatctt aa | 1512 |

SEQ ID NO: 68
T. halophila

| | |
|---|---|
| MASMISLLLG FVVSSFLFIF FLKKLLFFFS RHKMSEVSRL PSVPVPGFPL IGNLLQLKEK | 60 |
| KPHKTFTKWS ELYGPIYSIK MGSSSLIVLN SIETAKEAMV SRFSSISTRK LSNALTVLTC | 120 |
| NKSMVATSDY DDFHKFVKRC LLNGLLGANA QERKRHYRDA LIENVTSKLH AHTRNHPQEP | 180 |
| VNFRAIFEHE LFGVALKQAF GKDVESIYVK ELGVTLSRDE IFKVLVHDMM EGAIDVDWRD | 240 |
| FFPYLKWIPN NSFEARIQQK HKRRLAVMNA LIQDRLNQND SESDDDCYLN FLMSEAKTLT | 300 |
| MEQIAILVWE TIIETADTTL VTTEWAMYEL AKHQSVQDRL FKEIQSVCGG EKIKEEQLPR | 360 |
| LPYVNGVFHE TLRKYSPAPL VPIRYAHEDT QIGGYHIPAG SEIAINIYGC NMDKKRWERP | 420 |
| EEWWPERFLE DRYESSDLHK TMAFGAGKRV CAGALQASLM AGIAIGRLVQ EFEWKLRDGE | 480 |
| EENVDTYGLT SQKLYPLMAI INPRRS | 506 |

SEQ ID NO: 69
Artificial Sequence

| | |
|---|---|
| aagcttacta gtaaaatgga catgatgggt attgaagctg ttccatttgc tactgctgtt | 60 |
| gttttgggtg gtatttcctt ggttgttttg atcttcatca gaagattcgt ttccaacaga | 120 |
| aagagatccg ttgaaggttt gccaccagtt ccagatattc caggtttacc attgattggt | 180 |
| aacttgttgc aattgaaaga aaagaagcca cataagacct ttgctagatg ggctgaaact | 240 |
| tacggtccaa ttttctctat tagaactggt gcttctacca tgatcgtctt gaattcttct | 300 |
| gaagttgcca aagaagctat ggtcactaga ttctcttcaa tctctaccag aaagttgtcc | 360 |
| aacgccttga agattttgac cttcgataag tgtatggttg ccacctctga ttacaacgat | 420 |
| tttcacaaaa tggtcaaggg tttcatcttg agaaacgttt taggtgctcc agcccaaaaa | 480 |
| agacatagat gtcatagaga taccttgatc gaaaacatct ctaagtactt gcatgcccat | 540 |
| gttaagactt ctccattgga accagttgtc ttgaagaaga ttttcgaatc cgaaattttc | 600 |
| ggtttggctt tgaaacaagc cttgggtaag gatatcgaat ccatctatgt tgaagaattg | 660 |
| ggtactacct tgtccagaga agaaattttt gccgttttgg ttgttgatcc aatggctggt | 720 |
| gctattgaag ttgattggag agattttttc ccatacttgt cctggattcc aaacaagtct | 780 |
| atggaaatga agatccaaag aatggatttt agaagaggtg ctttgatgaa ggccttgatt | 840 |
| ggtgaacaaa agaaaagaat cggttccggt gaagaaaaga actcctacat tgatttcttg | 900 |
| ttgtctgaag ctaccacttt gaccgaaaag caaattgcta tgttgatctg ggaaaccatc | 960 |
| atcgaaattt ccgatacaac tttggttacc tctgaatggg ctatgtacga attggctaaa | 1020 |
| gacccaaata gacaagaaat cttgtacaga gaaatccaca aggtttgcgg ttctaacaag | 1080 |
| ttgactgaag aaaacttgtc caagttgcca tacttgaact ctgttttcca cgaaaccttg | 1140 |
| agaaagtatt ctccagctcc aatggttcca gttagatatg ctcatgaaga tactcaattg | 1200 |
| ggtggttacc atattccagc tggttctcaa attgccatta acatctacgg ttgcaacatg | 1260 |
| aacaaaaagc aatgggaaaa tcctgaagaa tggaagccag aaagattctt ggacgaaaag | 1320 |
| tatgacttga tggacttgca taagactatg gcttttggtg gtggtaaaag agtttgtgct | 1380 |
| ggtgctttac aagcaatgtt gattgcttgc acttccatcg gtagattcgt tcaagaattt | 1440 |
| gaatggaagt tgatgggtgg tgaagaagaa aacgttgata ctgttgcttt gacctcccaa | 1500 |
| aaattgcatc caatgcaagc cattattaag gccagagaat gactcgagcc gcgg | 1554 |

SEQ ID NO: 70
V. vinifera

| | |
|---|---|
| MDMMGIEAVP FATAVVLGGI SLVVLIFIRR FVSNRKRSVE GLPPVPDIPG LPLIGNLLQL | 60 |
| KEKKPHKTFA RWAETYGPIF SIRTGASTMI VLNSSEVAKE AMVTRFSSIS TRKLSNALKI | 120 |
| LTFDKCMVAT SDYNDFHKMV KGFILRNVLG APAQKRHRCH RDTLIENISK YLHAHVKTSP | 180 |
| LEPVVLKKIF ESEIFGLALK QALGKDIESI YVEELGTTLS REEIFAVLVV DPMAGAIEVD | 240 |
| WRDFFPYLSW IPNKSMEMKI QRMDFRRGAL MKALIGEQKK RIGSGEEKNS YIDFLLSEAT | 300 |
| TLTEKQIAML IWETIIEISD TTLVTSEWAM YELAKDPNRQ EILYREIHKV CGSNKLTEEN | 360 |
| LSKLPYLNSV FHETLRKYSP APMVPVRYAH EDTQLGGYHI PAGSQIAINI YGCNMNKKQW | 420 |
| ENPEEWKPER FLDEKYDLMD LHKTMAFGGG KRVCAGALQA MLIACTSIGR FVQEFEWKLM | 480 |
| GGEEENVDTV ALTSQKLHPM QAIIKARE | 508 |

SEQ ID NO: 71
Artificial Sequence

| | |
|---|---|
| aagcttaaaa tgagtaagtc taatagtatg aattctacat cacacgaaac cctttttcaa | 60 |
| caattggtct tgggtttgga ccgtatgcca ttgatggatg ttcactggtt gatctacgtt | 120 |
| gctttcggcg catggttatg ttcttatgtg atacatgttt tatcatcttc ctctacagta | 180 |
| aaagtgccag ttgttggata caggtctgta ttcgaaccta catggttgct tagacttaga | 240 |
| ttcgtctggg aaggtggctc tatcataggt caagggtaca ataagtttaa agactctatt | 300 |
| ttccaagtta ggaaattggg aactgatatt gtcattatac cacctaacta tattgatgaa | 360 |
| gtgagaaaat tgtcacagga caagactaga tcagttgaac ctttcattaa tgattttgca | 420 |
| ggtcaataca caagaggcat ggttttcttg caatctgact tacaaaaccg tgttatacaa | 480 |
| caaagactaa ctccaaaatt ggtttccttg accaaggtca tgaaggaaga gttggattat | 540 |
| gctttaacaa aagagatgcc tgatatgaaa aatgacgaat gggtagaagt agatatcagt | 600 |
| agtataatgg tgagattgat ttccaggatc tccgccagag tctttctagg gcctgaacac | 660 |
| tgtcgtaacc aggaatggtt gactactaca gcagaatatt cagaatcact tttcattaca | 720 |
| gggtttatct taagagttgt acctcatatc ttaagaccat tcatcgcccc tctattacct | 780 |
| tcatacagga ctctacttag aaacgtttca agtggtagaa gagtcatcgg tgacatcata | 840 |
| agatctcagc aaggggatgg taacgaagat atactttcct ggatgagaga tgctgccaca | 900 |
| ggagaggaaa agcaaatcga taacattgct cagagaatgt taattctttc tttagcatca | 960 |
| atccacacta ctgcgatgac catgacacat gccatgtacg atctatgtgc ttgccctgag | 1020 |
| tacattgaac cattaagaga tgaagttaaa tctgttgttg gggcttctgg ctgggacaag | 1080 |
| acagcgttaa acagatttca taagttggac tccttcctaa aagagtcaca aagattcaac | 1140 |
| ccagtattct tattgacatt caatagaatc taccatcaat ctatgacctt atcagatggc | 1200 |
| actaacattc catctggaac acgtattgct gttccatcac acgcaatgtt gcaagattct | 1260 |
| gcacatgtcc caggtccaac cccacctact gaatttgatg gattcagata tagtaagata | 1320 |
| cgttctgata gtaactacgc acaaaagtac ctattctcca tgaccgattc ttcaaacatg | 1380 |
| gctttcggat acggcaagta tgcttgtcca ggtagatttt acgcgtctaa tgagatgaaa | 1440 |
| ctaacattag ccattttgtt gctacaattt gagttcaaac taccagatgg taaaggtcgt | 1500 |
| cctagaaata tcactatcga ttctgatatg attccagacc caagagctag actttgcgtc | 1560 |
| agaaaaagat cacttagaga tgaatgaccg cgg | 1593 |

SEQ ID NO: 72
G. fujikuroi

| | |
|---|---|
| MSKSNSMNST SHETLFQQLV LGLDRMPLMD VHWLIYVAFG AWLCSYVIHV LSSSSTVKVP | 60 |
| VVGYRSVFEP TWLLRLRFVW EGGSIIGQGY NKFKDSIFQV RKLGTDIVII PPNYIDEVRK | 120 |
| LSQDKTRSVE PFINDFAGQY TRGMVFLQSD LQNRVIQQRL TPKLVSLTKV MKEELDYALT | 180 |
| KEMPDMKNDE WVEVDISSIM VRLISRISAR VFLGPEHCRN QEWLTTTAEY SESLFITGFI | 240 |
| LRVVPHILRP FIAPLLPSYR TLLRNVSSGR RVIGDIIRSQ QGDGNEDILS WMRDAATGEE | 300 |
| KQIDNIAQRM LILSLASIHT TAMTMTHAMY DLCACPEYIE PLRDEVKSVV GASGWDKTAL | 360 |
| NRFHKLDSFL KESQRFNPVF LLTFNRIYHQ SMTLSDGTNI PSGTRIAVPS HAMLQDSAHV | 420 |
| PGPTPPTEFD GFRYSKIRSD SNYAQKYLFS MTDSSNMAFG YGKYACPGRF YASNEMKLTL | 480 |
| AILLLQFEFK LPDGKGRPRN ITIDSDMIPD PRARLCVRKR SLRDE | 525 |

SEQ ID NO: 73
Artificial Sequence

| | |
|---|---|
| aagcttaaaa tggaagatcc tactgtctta tatgcttgtc ttgccattgc agttgcaact | 60 |
| ttcgttgtta gatggtacag agatccattg agatccatcc caacagttgg tggttccgat | 120 |
| ttgcctattc tatcttacat cggcgcacta agatggacaa gacgtggcag agagatactt | 180 |
| caagagggat atgatggcta cagaggatct acattcaaaa tcgcgatgtt agaccgttgg | 240 |
| atcgtgatcg caaatggtcc taaactagct gatgaagtca gacgtagacc agatgaagag | 300 |
| ttaaacttta tggacggatt aggagcattc gtccaaacta agtacacctt aggtgaagct | 360 |
| attcataacg atccatacca tgtcgatatc ataagagaaa aactaacaag aggccttcca | 420 |
| gccgtgcttc ctgatgtcat tgaagagttg acacttgcgg ttagacagta cattccaaca | 480 |
| gaaggtgatg aatgggtgtc cgtaaactgt tcaaaggccg caagagatat tgttgctaga | 540 |
| gcttctaata gagtctttgt aggtttgcct gcttgcagaa accaaggtta cttagatttg | 600 |
| gcaatagact ttacattgtc tgttgtcaag gatagagcca tcatcaatat gtttccagaa | 660 |
| ttgttgaagc caatagttgg cagagttgta ggtaacgcca ccagaaatgt tcgtagagct | 720 |
| gttccttttg ttgctccatt ggtggaggaa agacgtagac ttatggaaga gtacggtgaa | 780 |
| gactggtctg aaaaacctaa tgatatgtta cagtggataa tggatgaagc tgcatccaga | 840 |
| gatagttcag tgaaggcaat cgcagagaga ttgttaatgg tgaacttcgc ggctattcat | 900 |
| acctcatcaa acactatcac tcatgctttg taccaccttg ccgaaatgcc tgaaactttg | 960 |
| caaccactta gagaagagat cgaaccatta gtcaaagagg agggctggac caaggctgct | 1020 |
| atgggaaaaa tgtggtggtt agattcattt ctaagagaat ctcaaagata caatggcatt | 1080 |
| aacatcgtat ctttaactag aatggctgac aaagatatta cattgagtga tggcacattt | 1140 |
| ttgccaaaag gtactctagt ggccgttcca gcgtattcta ctcatagaga tgatgctgtc | 1200 |
| tacgctgatg ccttagtatt cgatcctttc agattctcac gtatgagagc gagagaaggt | 1260 |
| gaaggtacaa agcaccagtt cgttaatact tcagtcgagt acgttccatt tggtcacgga | 1320 |
| aagcatgctt gtccaggaag attcttcgcc gcaaacgaat tgaaagcaat gttggcttac | 1380 |
| attgttctaa actatgatgt aaagttgcct ggtgacggta aacgtccatt gaacatgtat | 1440 |
| tggggtccaa cagttttgcc tgcaccagca ggccaagtat tgttcagaaa gagacaagtt | 1500 |
| agtctataac cgcgg | 1515 |

SEQ ID NO: 74
T. versicolor

| | |
|---|---|
| MEDPTVLYAC LAIAVATFVV RWYRDPLRSI PTVGGSDLPI LSYIGALRWT RRGREILQEG | 60 |
| YDGYRGSTFK IAMLDRWIVI ANGPKLADEV RRRPDEELNF MDGLGAFVQT KYTLGEAIHN | 120 |
| DPYHVDIIRE KLTRGLPAVL PDVIEELTLA VRQYIPTEGD EWVSVNCSKA ARDIVARASN | 180 |
| RVFVGLPACR NQGYLDLAID FTLSVVKDRA IINMFPELLK PIVGRVVGNA TRNVRRAVPF | 240 |
| VAPLVEERRR LMEEYGEDWS EKPNDMLQWI MDEAASRDSS VKAIAERLLM VNFAAIHTSS | 300 |
| NTITHALYHL AEMPETLQPL REEIEPLVKE EGWTKAAMGK MWWLDSFLRE SQRYNGINIV | 360 |
| SLTRMADKDI TLSDGTFLPK GTLVAVPAYS THRDDAVYAD ALVFDPFRFS RMRAREGEGT | 420 |
| KHQFVNTSVE YVPFGHGKHA CPGRFFAANE LKAMLAYIVL NYDVKLPGDG KRPLNMYWGP | 480 |
| TVLPAPAGQV LFRKRQVSL | 499 |

SEQ ID NO: 75
Artificial Sequence

| | |
|---|---|
| atggcatttt tctctatgat ttcaattttg ttgggatttg ttatttcttc tttcatcttc | 60 |
| atctttttct tcaaaaagtt acttagtttt agtaggaaaa acatgtcaga agtttctact | 120 |
| ttgccaagtg ttccagtagt gcctggtttt ccagttattg ggaatttgtt gcaactaaag | 180 |
| gagaaaaagc ctcataaaac tttcactaga tggtcagaga tatatggacc tatctactct | 240 |
| ataaagatgg gttcttcatc tcttattgta ttgaacagta cagaaactgc taaggaagca | 300 |
| atggtcacta gattttcatc aatatctacc agaaaattgt caaacgccct aacagttcta | 360 |
| acctgcgata agtctatggt cgccacttct gattatgatg acttccacaa attagttaag | 420 |
| agatgtttgc taaatggact tcttggtgct aatgctcaaa agagaaaaag acactacaga | 480 |
| gatgctttga ttgaaaatgt gagttccaag ctacatgcac acgctagaga tcatccacaa | 540 |
| gagccagtta actttagagc aattttcgaa cacgaattgt ttggtgtagc attaaagcaa | 600 |
| gccttcggta aagacgtaga atccatatac gtcaaggagt taggcgtaac attatcaaaa | 660 |
| gatgaaatct ttaaggtgct tgtacatgat atgatggagg gtgcaattga tgtagattgg | 720 |
| agagatttct tcccatattt gaaatggatc cctaataagt cttttgaagc taggatacaa | 780 |
| caaaagcaca agagaagact agctgttatg aacgcactta tacaggacag attgaagcaa | 840 |
| aatgggtctg aatcagatga tgattgttac cttaacttct taatgtctga ggctaaaaca | 900 |
| ttgactaagg aacagatcgc aatccttgtc tgggaaacaa tcattgaaac agcagatact | 960 |
| accttagtca caactgaatg ggccatatac gagctagcca aacatccatc tgtgcaagat | 1020 |
| aggttgtgta aggagatcca gaacgtgtgt ggtggagaga aattcaagga agagcagttg | 1080 |
| tcacaagttc cttaccttaa cggcgttttc catgaaacct tgagaaaata ctcacctgca | 1140 |
| ccattagttc ctattagata cgcccacgaa gatacacaaa tcggtggcta ccatgttcca | 1200 |
| gctgggtccg aaattgctat aaacatctac gggtgcaaca tggacaaaaa gagatgggaa | 1260 |
| agaccagaag attggtggcc agaaagattc ttagatgatg gcaaatatga aacatctgat | 1320 |
| ttgcataaaa caatggcttt cggagctggc aaaagagtgt gtgccggtgc tctacaagcc | 1380 |
| tccctaatgg ctggtatcgc tattggtaga ttggtccaag agttcgaatg gaaacttaga | 1440 |
| gatggtgaag aggaaaatgt cgatacttat gggttaacat ctcaaaagtt atacccacta | 1500 |
| atggcaatca tcaatcctag aagatcctaa | 1530 |

SEQ ID NO: 76
A. thaliana

| | |
|---|---|
| MAFFSMISIL LGFVISSFIF IFFFKKLLSF SRKNMSEVST LPSVPVVPGF PVIGNLLQLK | 60 |
| EKKPHKTFTR WSEIYGPIYS IKMGSSSLIV LNSTETAKEA MVTRFSSIST RKLSNALTVL | 120 |
| TCDKSMVATS DYDDFHKLVK RCLLNGLLGA NAQKRKRHYR DALIENVSSK LHAHARDHPQ | 180 |
| EPVNFRAIFE HELFGVALKQ AFGKDVESIY VKELGVTLSK DEIFKVLVHD MMEGAIDVDW | 240 |
| RDFFPYLKWI PNKSFEARIQ QKHKRRLAVM NALIQDRLKQ NGSESDDDCY LNFLMSEAKT | 300 |
| LTKEQIAILV WETIIETADT TLVTTEWAIY ELAKHPSVQD RLCKEIQNVC GGEKFKEEQL | 360 |
| SQVPYLNGVF HETLRKYSPA PLVPIRYAHE DTQIGGYHVP AGSEIAINIY GCNMDKKRWE | 420 |
| RPEDWWPERF LDDGKYETSD LHKTMAFGAG KRVCAGALQA SLMAGIAIGR LVQEFEWKLR | 480 |
| DGEEENVDTY GLTSQKLYPL MAIINPRRS | 509 |

SEQ ID NO: 77
Artificial Sequence

| | |
|---|---|
| atgcaatcag attcagtcaa agtctctcca tttgatttgg tttccgctgc tatgaatggc | 60 |
| aaggcaatgg aaaagttgaa cgctagtgaa tctgaagatc caacaacatt gcctgcacta | 120 |
| aagatgctag ttgaaaatag agaattgttg acactgttca caacttcctt cgcagttctt | 180 |
| attgggtgtc ttgtatttct aatgtggaga cgttcatcct ctaaaaagct ggtacaagat | 240 |
| ccagttccac aagttatcgt tgtaaagaag aaagagaagg agtcagaggt tgatgacggg | 300 |
| aaaaagaaag tttctatttt ctacggcaca caaacaggaa ctgccgaagg ttttgctaaa | 360 |
| gcattagtcg aggaagcaaa agtgagatat gaaaagacct ctttcaaggt tatcgatcta | 420 |
| gatgactacg ctgcagatga tgatgaatat gaggaaaaac tgaaaaagga atccttagcc | 480 |
| ttcttcttct tggccacata cggtgatggt gaacctactg ataatgctgc taacttctac | 540 |
| aagtggttca cagaaggcga cgataaaggt gaatggctga aaaagttaca atacggagta | 600 |
| tttggtttag gtaacagaca atatgaacat ttcaacaaga tcgctattgt agttgatgat | 660 |
| aaacttactg aaatgggagc caaaagatta gtaccagtag gattagggga tgatgatcag | 720 |
| tgtatagaag atgacttcac cgcctggaag gaattggtat ggccagaatt ggatcaactt | 780 |
| ttaagggacg aagatgatac ttctgtgact accccataca ctgcagccgt attggagtac | 840 |
| agagtggttt accatgataa accagcagac tcatatgctg aagatcaaac ccatacaaac | 900 |
| ggtcatgttg ttcatgatgc acagcatcct tcaagatcta atgtggcttt caaaaaggaa | 960 |
| ctacacacct ctcaatcaga taggtcttgt actcacttag aattcgatat ttctcacaca | 1020 |
| ggactgtctt acgaaactgg cgatcacgtt ggcgtttatt ccgagaactt gtccgaagtt | 1080 |
| gtcgatgaag cactaaaact gttagggtta tcaccagaca catacttctc agtccatgct | 1140 |
| gataaggagg atgggacacc tatcggtggt gcttcactac caccaccttt tcctccttgc | 1200 |
| acattgagag acgctctaac cagatacgca gatgtcttat cctcacctaa aaaggtagct | 1260 |
| ttgctggcat tggctgctca tgctagtgat cctagtgaag ccgataggtt aaagttcctg | 1320 |
| gcttcaccag ccggaaaaga tgaatatgca caatggatcg tcgccaacca acgttctttg | 1380 |
| ctagaagtga tgcaaagttt tccatctgcc aagcctccat taggtgtgtt cttcgcagca | 1440 |
| gtagctccac gtttacaacc aagatactac tctatcagtt catctcctaa gatgtctcct | 1500 |
| aacagaatac atgttacatg tgctttggtg tacgagacta ctccagcagg cagaattcac | 1560 |
| agaggattgt gttcaacctg gatgaaaaat gctgtccctt taacagagtc acctgattgc | 1620 |
| tctcaagcat ccattttcgt tagaacatca aatttcagac ttccagtgga tccaaaagtt | 1680 |
| ccagtcatta tgataggacc aggcactggt cttgccccat tcaggggctt tcttcaagag | 1740 |
| agattggcct tgaaggaatc tggtacagaa ttgggttctt ctatcttttt ctttggttgc | 1800 |
| cgtaatagaa aagttgactt tatctacgag gacgagctta acaattttgt tgagacagga | 1860 |
| gcattgtcag aattgatcgt cgcattttca agagaaggga ctgccaaaga gtacgttcag | 1920 |
| cacaagatga gtcaaaaagc ctccgatata tggaaacttc taagtgaagg tgcctatctt | 1980 |
| tatgtctgtg gcgatgcaaa gggcatggcc aaggatgtcc atagaactct gcatacaatt | 2040 |
| gttcaggaac aagggagtct ggattcttcc aaggctgaat tgtacgtcaa aaacttacag | 2100 |
| atgtctggaa gatacttaag agatgtttgg taa | 2133 |

SEQ ID NO: 78
S. rebaudiana

| | |
|---|---|
| MQSDSVKVSP FDLVSAAMNG KAMEKLNASE SEDPTTLPAL KMLVENRELL TLFTTSFAVL | 60 |
| IGCLVFLMWR RSSSKKLVQD PVPQVIVVKK KEKESEVDDG KKKVSIFYGT QTGTAEGFAK | 120 |
| ALVEEAKVRY EKTSFKVIDL DDYAADDDEY EEKLKKESLA FFFLATYGDG EPTDNAANFY | 180 |
| KWFTEGDDKG EWLKKLQYGV FGLGNRQYEH FNKIAIVVDD KLTEMGAKRL VPVGLGDDDQ | 240 |
| CIEDDFTAWK ELVWPELDQL LRDEDDTSVT TPYTAAVLEY RVVYHDKPAD SYAEDQTHTN | 300 |
| GHVVHDAQHP SRSNVAFKKE LHTSQSDRSC THLEFDISHT GLSYETGDHV GVYSENLSEV | 360 |
| VDEALKLLGL SPDTYFSVHA DKEDGTPIGG ASLPPPFPPC TLRDALTRYA DVLSSPKKVA | 420 |
| LLALAAHASD PSEADRLKFL ASPAGKDEYA QWIVANQRSL LEVMQSFPSA KPPLGVFFAA | 480 |
| VAPRLQPRYY SISSSPKMSP NRIHVTCALV YETTPAGRIH RGLCSTWMKN AVPLTESPDC | 540 |
| SQASIFVRTS NFRLPVDPKV PVIMIGPGTG LAPFRGFLQE RLALKESGTE LGSSIFFFGC | 600 |
| RNRKVDFIYE DELNNFVETG ALSELIVAFS REGTAKEYVQ HKMSQKASDI WKLLSEGAYL | 660 |
| YVCGDAKGMA KDVHRTLHTI VQEQGSLDSS KAELYVKNLQ MSGRYLRDVW | 710 |

SEQ ID NO: 79
S. grosvenorii

| | |
|---|---|
| atgaaggtca gtccattcga attcatgtcc gctattatca agggtagaat ggacccatct | 60 |
| aactcctcat ttgaatctac tggtgaagtt gcctccgtta tctttgaaaa cagagaattg | 120 |
| gttgccatct tgaccacttc tattgctgtt atgattggtt gcttcgttgt cttgatgtgg | 180 |
| agaagagctg gttctagaaa ggttaagaat gtcgaattgc caaagccatt gattgtccat | 240 |
| gaaccagaac ctgaagttga agatggtaag aagaaggttt ccatcttctt cggtactcaa | 300 |
| actggtactg ctgaaggttt tgctaaggct ttggctgatg aagctaaagc tagatacgaa | 360 |
| aaggctacct tcagagttgt tgatttggat gattatgctg ccgatgatga ccaatacgaa | 420 |
| gaaaaattga agaacgaatc cttcgccgtt ttcttgttgg ctacttatgg tgatggtgaa | 480 |
| cctactgata atgctgctag attttacaag tggttcgccg aaggtaaaga aagaggtgaa | 540 |
| tggttgcaaa acttgcacta tgctgttttt ggtttgggta acagacaata cgaacacttc | 600 |
| aacaagattg ctaaggttgc cgacgaatta ttggaagctc aaggtggtaa tagattggtt | 660 |
| aaggttggtt taggtgatga cgatcaatgc atcgaagatg atttttctgc ttggagagaa | 720 |
| tctttgtggc cagaattgga tatgttgttg agagatgaag atgatgctac tactgttact | 780 |
| actccatata ctgctgctgt cttggaatac agagttgtct ttcatgattc tgctgatgtt | 840 |
| gctgctgaag ataagtcttg gattaacgct aatggtcatg ctgttcatga tgctcaacat | 900 |
| ccattcagat ctaacgttgt cgtcagaaaa gaattgcata cttctgcctc tgatagatcc | 960 |
| tgttctcatt tggaattcaa catttccggt tccgctttga attacgaaac tggtgatcat | 1020 |
| gttggtgtct actgtgaaaa cttgactgaa actgttgatg aagccttgaa cttgttgggt | 1080 |
| ttgtctccag aaacttactt ctctatctac accgataacg aagatggtac tccattgggt | 1140 |
| ggttcttcat tgccaccacc atttccatca tgtactttga gaactgcttt gaccagatac | 1200 |
| gctgatttgt tgaactctcc aaaaaagtct gctttgttgg ctttagctgc tcatgcttct | 1260 |
| aatccagttg aagctgatag attgagatac ttggcttctc cagctggtaa agatgaatat | 1320 |
| gcccaatctg ttatcggttc ccaaaagtct ttgttggaag ttatggctga attcccatct | 1380 |
| gctaaaccac cattaggtgt tttttttgct gctgttgctc caagattgca acctagattc | 1440 |
| tactccattt catcctctcc aagaatggct ccatctagaa tccatgttac ttgtgctttg | 1500 |
| gtttacgata agatgccaac tggtagaatt cataagggtg tttgttctac ctggatgaag | 1560 |
| aattctgttc caatggaaaa gtcccatgaa tgttcttggg ctccaatttt cgttagacaa | 1620 |
| tccaatttta agttgccagc cgaatccaag gttccaatta tcatggttgg tccaggtact | 1680 |
| ggtttggctc cttttagagg ttttttacaa gaaagattgg ccttgaaaga atccggtgtt | 1740 |
| gaattgggtc catccatttt gtttttcggt tgcagaaaca gaagaatgga ttacatctac | 1800 |
| gaagatgaat tgaacaactt cgttgaaacc ggtgctttgt ccgaattggt tattgctttt | 1860 |
| tctagagaag gtcctaccaa agaatacgtc caacataaga tggctgaaaa ggcttctgat | 1920 |
| atctggaact tgatttctga aggtgcttac ttgtacgttt gtggtgatgc taaaggtatg | 1980 |
| gctaaggatg ttcatagaac cttgcatacc atcatgcaag aacaaggttc tttggattct | 2040 |
| tccaaagctg aatccatggt caagaacttg caaatgaatg gtagatactt aagagatgtt | 2100 |
| tggtaa | 2106 |

SEQ ID NO: 80
S. grosvenorii

| | |
|---|---|
| MKVSPFEFMS AIIKGRMDPS NSSFESTGEV ASVIFENREL VAILTTSIAV MIGCFVVLMW | 60 |
| RRAGSRKVKN VELPKPLIVH EPEPEVEDGK KKVSIFFGTQ TGTAEGFAKA LADEAKARYE | 120 |
| KATFRVVDLD DYAADDDQYE EKLKNESFAV FLLATYGDGE PTDNAARFYK WFAEGKERGE | 180 |
| WLQNLHYAVF GLGNRQYEHF NKIAKVADEL LEAQGGNRLV KVGLGDDDQC IEDDFSAWRE | 240 |
| SLWPELDMLL RDEDDATTVT TPYTAAVLEY RVVFHDSADV AAEDKSWINA NGHAVHDAQH | 300 |
| PFRSNVVVRK ELHTSASDRS CSHLEFNISG SALNYETGDH VGVYCENLTE TVDEALNLLG | 360 |
| LSPETYFSIY TDNEDGTPLG GSSLPPPFPS CTLRTALTRY ADLLNSPKKS ALLALAAHAS | 420 |
| NPVEADRLRY LASPAGKDEY AQSVIGSQKS LLEVMAEFPS AKPPLGVFFA AVAPRLQPRF | 480 |
| YSISSSPRMA PSRIHVTCAL VYDKMPTGRI HKGVCSTWMK NSVPMEKSHE CSWAPIFVRQ | 540 |
| SNFKLPAESK VPIIMVGPGT GLAPFRGFLQ ERLALKESGV ELGPSILFFG CRNRRMDYIY | 600 |
| EDELNNFVET GALSELVIAF SREGPTKEYV QHKMAEKASD IWNLISEGAY LYVCGDAKGM | 660 |
| AKDVHRTLHT IMQEQGSLDS SKAESMVKNL QMNGRYLRDV W | 701 |

SEQ ID NO: 81
Artificial Sequence

| | |
|---|---|
| atggcagaat tagatacact tgatatagta gtattaggtg ttatcttttt gggtactgtg | 60 |
| gcatacttta ctaagggtaa attgtggggt gttaccaagg atccatacgc taacggattc | 120 |
| gctgcaggtg gtgcttccaa gcctggcaga actagaaaca tcgtcgaagc tatggaggaa | 180 |
| tcaggtaaaa actgtgttgt tttctacggc agtcaaacag gtacagcgga ggattacgca | 240 |
| tcaagacttg caaaggaagg aaagtccaga ttcggtttga acactatgat cgccgatcta | 300 |
| gaagattatg acttcgataa cttagacact gttccatctg ataacatcgt tatgtttgta | 360 |
| ttggctactt acggtgaagg cgaaccaaca gataacgccg tggatttcta tgagttcatt | 420 |
| actggcgaag atgcctcttt caatgagggc aacgatcctc cactaggtaa cttgaattac | 480 |
| gttgcgttcg gtctgggcaa caatacctac gaacactaca actcaatggt caggaacgtt | 540 |
| aacaaggctc tagaaaagtt aggagctcat agaattggag aagcaggtga gggtgacgac | 600 |
| ggagctggaa ctatggaaga ggacttttta gcttggaaag atccaatgtg ggaagccttg | 660 |
| gctaaaaaga tgggcttgga ggaaagagaa gctgtatatg aacctatttt cgctatcaat | 720 |
| gagagagatg atttgacccc tgaagcgaat gaggtatact tgggagaacc taataagcta | 780 |
| cacttggaag gtacagcgaa aggtccattc aactcccaca acccatatat cgcaccaatt | 840 |
| gcagaatcat acgaactttt ctcagctaag gatagaaatt gtctgcatat ggaaattgat | 900 |
| atttctggta gtaatctaaa gtatgaaaca ggcgaccata tcgcgatctg gcctaccaac | 960 |
| ccaggtgaag aggtcaacaa atttcttgac attctagatc tgtctggtaa gcaacattcc | 1020 |
| gtcgtaacag tgaaagcctt agaacctaca gccaaagttc cttttccaaa tccaactacc | 1080 |
| tacgatgcta tattgagata ccatctggaa atatgcgctc cagtttctag acagtttgtc | 1140 |
| tcaactttag cagcattcgc ccctaatgat gatatcaaag ctgagatgaa ccgtttggga | 1200 |
| tcagacaaag attacttcca cgaaaagaca ggaccacatt actacaatat cgctagattt | 1260 |
| ttggcctcag tctctaaagg tgaaaaatgg acaaagatac cattttctgc tttcatagaa | 1320 |
| ggccttacaa aactacaacc aagatactat tctatctctt cctctagttt agttcagcct | 1380 |
| aaaaagatta gtattactgc tgttgtcgaa tctcagcaaa ttccaggtag agatgaccca | 1440 |
| ttcagaggtg tagcgactaa ctacttgttc gctttgaagc agaaacaaaa cggtgatcca | 1500 |
| aatccagctc cttttggcca atcatacgag ttgacaggac caaggaataa gtatgatggt | 1560 |
| atacatgttc cagtccatgt aagacattct aactttaagc taccatctga tccaggcaaa | 1620 |
| cctattatca tgatcggtcc aggtaccggt gttgcccctt ttagaggctt cgtccaagag | 1680 |
| agggcaaaac aagccagaga tggtgtagaa gttggtaaaa cactgctgtt ctttggatgt | 1740 |
| agaaagagta cagaagattt catgtatcaa aaagagtggc aagagtacaa ggaagctctt | 1800 |
| ggcgacaaat tcgaaatgat tacagctttt tcaagagaag gatctaaaaa ggtttatgtt | 1860 |
| caacacagac tgaaggaaag atcaaaggaa gtttctgatc ttctatccca aaaagcatac | 1920 |
| ttctacgttt gcggagacgc cgcacatatg gcacgtgaag tgaacactgt gttagcacag | 1980 |
| atcatagcag aaggccgtgg tgtatcagaa gccaagggtg aggaaattgt caaaaacatg | 2040 |
| agatcagcaa atcaatacca agtgtgttct gatttcgtaa ctttacactg taaagagaca | 2100 |
| acatacgcga attcagaatt gcaagaggat gtctggagtt aa | 2142 |

SEQ ID NO: 82
G. fujikuroi

| | |
|---|---|
| MAELDTLDIV VLGVIFLGTV AYFTKGKLWG VTKDPYANGF AAGGASKPGR TRNIVEAMEE | 60 |
| SGKNCVVFYG SQTGTAEDYA SRLAKEGKSR FGLNTMIADL EDYDFDNLDT VPSDNIVMFV | 120 |
| LATYGEGEPT DNAVDFYEFI TGEDASFNEG NDPPLGNLNY VAFGLGNNTY EHYNSMVRNV | 180 |
| NKALEKLGAH RIGEAGEGDD GAGTMEEDFL AWKDPMWEAL AKKMGLEERE AVYEPIFAIN | 240 |
| ERDDLTPEAN EVYLGEPNKL HLEGTAKGPF NSHNPYIAPI AESYELFSAK DRNCLHMEID | 300 |
| ISGSNLKYET GDHIAIWPTN PGEEVNKFLD ILDLSGKQHS VVTVKALEPT AKVPFPNPTT | 360 |
| YDAILRYHLE ICAPVSRQFV STLAAFAPND DIKAEMNRLG SDKDYFHEKT GPHYYNIARF | 420 |
| LASVSKGEKW TKIPFSAFIE GLTKLQPRYY SISSSSLVQP KKISITAVVE SQQIPGRDDP | 480 |
| FRGVATNYLF ALKQKQNGDP NPAPFGQSYE LTGPRNKYDG IHVPVHVRHS NFKLPSDPGK | 540 |
| PIIMIGPGTG VAPFRGFVQE RAKQARDGVE VGKTLLFFGC RKSTEDFMYQ KEWQEYKEAL | 600 |
| GDKFEMITAF SREGSKKVYV QHRLKERSKE VSDLLSQKAY FYVCGDAAHM AREVNTVLAQ | 660 |
| IIAEGRGVSE AKGEEIVKNM RSANQYQVCS DFVTLHCKET TYANSELQED VWS | 713 |

SEQ ID NO: 83
S. rebaudiana

| | |
|---|---|
| atgcaatcgg aatccgttga agcatcgacg attgatttga tgactgctgt tttgaaggac | 60 |
| acagtgatcg atacagcgaa cgcatctgat aacggagact caaagatgcc gccggcgttg | 120 |
| gcgatgatgt tcgaaattcg tgatctgttg ctgattttga ctacgtcagt tgctgttttg | 180 |
| gtcggatgtt tcgttgtttt ggtgtggaag agatcgtccg ggaagaagtc cggcaaggaa | 240 |
| ttggagccgc cgaagatcgt tgtgccgaag aggcggctgg agcaggaggt tgatgatggt | 300 |
| aagaagaagg ttacgatttt cttcggaaca caaactggaa cggctgaagg tttcgctaag | 360 |
| gcacttttcg aagaagcgaa agcgcgatat gaaaaggcag cgtttaaagt gattgatttg | 420 |
| gatgattatg ctgctgattt ggatgagtat gcagagaagc tgaagaagga aacatatgct | 480 |
| ttcttcttct tggctacata tggagatggt gagccaactg ataatgctgc caaattttat | 540 |
| aaatggttta ctgagggaga cgagaaaggc gtttggcttc aaaaacttca atatggagta | 600 |
| tttggtcttg gcaacagaca atatgaacat ttcaacaaga ttggaatagt ggttgatgat | 660 |
| ggtctcaccg agcagggtgc aaaacgcatt gttcccgttg gtcttggaga cgacgatcaa | 720 |
| tcaattgaag acgatttttc ggcatggaaa gagttagtgt ggcccgaatt ggatctattg | 780 |
| cttcgcgatg aagatgacaa agctgctgca actccttaca cagctgcaat ccctgaatac | 840 |
| cgcgtcgtat ttcatgacaa acccgatgcg ttttctgatg atcatactca aaccaatggt | 900 |
| catgctgttc atgatgctca acatccatgc agatccaatg tggctgttaa aaaagagctt | 960 |
| catactcctg aatccgatcg ttcatgcaca catcttgaat ttgacatttc tcacactgga | 1020 |
| ttatcttatg aaactgggga tcatgttggt gtatactgtg aaaacctaat tgaagtagtg | 1080 |
| gaagaagctg ggaaattgtt aggattatca acagatactt atttctcgtt acatattgat | 1140 |
| aacgaagatg gttcaccact tggtggacct tcattacaac ctccttttcc tccttgtact | 1200 |
| ttaagaaaag cattgactaa ttatgcagat ctgttaagct ctcccaaaaa gtcaactttg | 1260 |
| cttgctctag ctgctcatgc ttccgatccc actgaagctg atcgtttaag atttcttgca | 1320 |
| tctcgcgagg gcaaggatga atatgctgaa tgggttgttg caaaccaaag aagtcttctt | 1380 |
| gaagtcatgg aagctttccc gtcagctaga ccgccacttg gtgttttctt tgcagcggtt | 1440 |
| gcaccgcgtt tacagcctcg ttactactct atttcttcct ccccaaagat ggaaccaaac | 1500 |
| aggattcatg ttacttgcgc gttggtttat gaaaaaactc ccgcaggtcg tatccacaaa | 1560 |
| ggaatctgct caacctggat gaagaacgct gtacctttga ccgaaagtca agattgcagt | 1620 |
| tgggcaccga tttttgttag aacatcaaac ttcagacttc caattgaccc gaaagtcccg | 1680 |
| gttatcatga ttggtcctgg aaccgggttg gctccattta ggggttttct tcaagaaaga | 1740 |
| ttggctctta aagaatccgg aaccgaactc gggtcatcta ttttattctt cggttgtaga | 1800 |
| aaccgcaaag tggattacat atatgagaat gaactcaaca actttgttga aaatggtgcg | 1860 |
| ctttctgagc ttgatgttgc tttctcccgc gatggcccga cgaaagaata cgtgcaacat | 1920 |
| aaaatgaccc aaaaggcttc tgaaatatgg aatatgcttt ctgagggagc atatttatat | 1980 |
| gtatgtggtg atgctaaagg catggctaaa gatgtacacc gtacacttca caccattgtg | 2040 |
| caagaacagg gaagtttgga ctcgtctaaa gcggagttgt atgtgaagaa tctacaaatg | 2100 |
| tcaggaagat acctccgtga tgtttggtaa | 2130 |

SEQ ID NO: 84
S. rebaudiana

| | |
|---|---|
| MQSESVEAST IDLMTAVLKD TVIDTANASD NGDSKMPPAL AMMFEIRDLL LILTTSVAVL | 60 |
| VGCFVVLVWK RSSGKKSGKE LEPPKIVVPK RRLEQEVDDG KKKVTIFFGT QTGTAEGFAK | 120 |
| ALFEEAKARY EKAAFKVIDL DDYAADLDEY AEKLKKETYA FFFLATYGDG EPTDNAAKFY | 180 |
| KWFTEGDEKG VWLQKLQYGV FGLGNRQYEH FNKIGIVVDD GLTEQGAKRI VPVGLGDDDQ | 240 |
| SIEDDFSAWK ELVWPELDLL LRDEDDKAAA TPYTAAIPEY RVVFHDKPDA FSDDHTQTNG | 300 |
| HAVHDAQHPC RSNVAVKKEL HTPESDRSCT HLEFDISHTG LSYETGDHVG VYCENLIEVV | 360 |
| EEAGKLLGLS TDTYFSLHID NEDGSPLGGP SLQPPFPPCT LRKALTNYAD LLSSPKKSTL | 420 |
| LALAAHASDP TEADRLRFLA SREGKDEYAE WVVANQRSLL EVMEAFPSAR PPLGVFFAAV | 480 |
| APRLQPRYYS ISSSPKMEPN RIHVTCALVY EKTPAGRIHK GICSTWMKNA VPLTESQDCS | 540 |
| WAPIFVRTSN FRLPIDPKVP VIMIGPGTGL APFRGFLQER LALKESGTEL GSSILFFGCR | 600 |
| NRKVDYIYEN ELNNFVENGA LSELDVAFSR DGPTKEYVQH KMTQKASEIW NMLSEGAYLY | 660 |
| VCGDAKGMAK DVHRTLHTIV QEQGSLDSSK AELYVKNLQM SGRYLRDVW | 709 |

SEQ ID NO: 85
Artificial Sequence

| | |
|---|---|
| atgcaatcta actccgtgaa gatttcgccg cttgatctgg taactgcgct gtttagcggc | 60 |
| aaggttttgg acacatcgaa cgcatcggaa tcgggagaat ctgctatgct gccgactata | 120 |
| gcgatgatta tggagaatcg tgagctgttg atgatactca caacgtcggt tgctgtattg | 180 |
| atcggatgcg ttgtcgtttt ggtgtggcgg agatcgtcta cgaagaagtc ggcgttggag | 240 |
| ccaccggtga ttgtggttcc gaagagagtg caagaggagg aagttgatga tggtaagaag | 300 |
| aaagttacgg ttttcttcgg cacccaaact ggaacagctg aaggcttcgc taaggcactt | 360 |
| gttgaggaag ctaaagctcg atatgaaaag gctgtcttta aagtaattga tttggatgat | 420 |
| tatgctgctg atgacgatga gtatgaggag aaactaaaga aagaatcttt ggcctttttc | 480 |
| tttttggcta cgtatggaga tggtgagcca acagataatg ctgccagatt ttataaatgg | 540 |
| tttactgagg gagatgcgaa aggagaatgg cttaataagc ttcaatatgg agtatttggt | 600 |
| ttgggtaaca gacaatatga acattttaac aagatcgcaa aagtggttga tgatggtctt | 660 |
| gtagaacagg gtgcaaagcg tcttgttcct gttggacttg gagatgatga tcaatgtatt | 720 |
| gaagatgact tcaccgcatg gaaagagtta gtatggccgg agttggatca attacttcgt | 780 |
| gatgaggatg acacaactgt tgctactcca tacacagctg ctgttgcaga atatcgcgtt | 840 |
| gtttttcatg aaaaaccaga cgcgctttct gaagattata gttatacaaa tggccatgct | 900 |
| gttcatgatg ctcaacatcc atgcagatcc aacgtggctg tcaaaaagga acttcatagt | 960 |
| cctgaatctg accggtcttg cactcatctt gaatttgaca tctcgaacac cggactatca | 1020 |
| tatgaaactg gggaccatgt tggagtttac tgtgaaaact tgagtgaagt tgtgaatgat | 1080 |
| gctgaaagat tagtaggatt accaccagac acttactcct ccatccacac tgatagtgaa | 1140 |
| gacgggtcgc cacttggcgg agcctcattg ccgcctcctt tcccgccatg cactttaagg | 1200 |
| aaagcattga cgtgttatgc tgatgttttg agttctccca agaagtcggc tttgcttgca | 1260 |
| ctagctgctc atgccaccga tcccagtgaa gctgatagat tgaaatttct tgcatccccc | 1320 |
| gccggaaagg atgaatattc tcaatggata gttgcaagcc aaagaagtct ccttgaagtc | 1380 |
| atggaagcat tcccgtcagc taagccttca cttggtgttt tctttgcatc tgttgccccg | 1440 |
| cgcttacaac caagatacta ctctatttct tcctcaccca agatggcacc ggataggatt | 1500 |
| catgttacat gtgcattagt ctatgagaaa acacctgcag gccgcatcca caaaggagtt | 1560 |
| tgttcaactt ggatgaagaa cgcagtgcct atgaccgaga gtcaagattg cagttgggcc | 1620 |
| ccaatatacg tccgaacatc caatttcaga ctaccatctg accctaaggt cccggttatc | 1680 |
| atgattggac ctggcactgg tttggctcct tttagaggtt tccttcaaga gcggttagct | 1740 |
| ttaaaggaag ccggaactga cctcggttta tccattttat tcttcggatg taggaatcgc | 1800 |
| aaagtggatt tcatatatga aaacgagctt aacaactttg tggagactgg tgctctttct | 1860 |
| gagcttattg ttgctttctc ccgtgaaggc ccgactaagg aatatgtgca acacaagatg | 1920 |
| agtgagaagg cttcggatat ctggaacttg ctttctgaag gagcatattt atacgtatgt | 1980 |
| ggtgatgcca aaggcatggc caaagatgta catcgaaccc tccacacaat tgtgcaagaa | 2040 |
| cagggatctc ttgactcgtc aaaggcagaa ctctacgtga agaatctaca aatgtcagga | 2100 |
| agatacctcc gtgacgtttg gtaa | 2124 |

SEQ ID NO: 86
S. rebaudiana

| | |
|---|---|
| MQSNSVKISP LDLVTALFSG KVLDTSNASE SGESAMLPTI AMIMENRELL MILTTSVAVL | 60 |
| IGCVVVLVWR RSSTKKSALE PPVIVVPKRV QEEEVDDGKK KVTVFFGTQT GTAEGFAKAL | 120 |
| VEEAKARYEK AVFKVIDLDD YAADDDEYEE KLKKESLAFF FLATYGDGEP TDNAARFYKW | 180 |
| FTEGDAKGEW LNKLQYGVFG LGNRQYEHFN KIAKVVDDGL VEQGAKRLVP VGLGDDDQCI | 240 |
| EDDFTAWKEL VWPELDQLLR DEDDTTVATP YTAAVAEYRV VFHEKPDALS EDYSYTNGHA | 300 |
| VHDAQHPCRS NVAVKKELHS PESDRSCTHL EFDISNTGLS YETGDHVGVY CENLSEVVND | 360 |
| AERLVGLPPD TYSSIHTDSE DGSPLGGASL PPPFPPCTLR KALTCYADVL SSPKKSALLA | 420 |
| LAAHATDPSE ADRLKFLASP AGKDEYSQWI VASQRSLLEV MEAFPSAKPS LGVFFASVAP | 480 |
| RLQPRYYSIS SSPKMAPDRI HVTCALVYEK TPAGRIHKGV CSTWMKNAVP MTESQDCSWA | 540 |
| PIYVRTSNFR LPSDPKVPVI MIGPGTGLAP FRGFLQERLA LKEAGTDLGL SILFFGCRNR | 600 |
| KVDFIYENEL NNFVETGALS ELIVAFSREG PTKEYVQHKM SEKASDIWNL LSEGAYLYVC | 660 |
| GDAKGMAKDV HRTLHTIVQE QGSLDSSKAE LYVKNLQMSG RYLRDVW | 707 |

SEQ ID NO: 87
Artificial Sequence

| | |
|---|---|
| atgtcctcca actccgattt ggtcagaaga ttggaatctg ttttgggtgt ttctttcggt | 60 |
| ggttctgtta ctgattccgt tgttgttatt gctaccacct ctattgcttt ggttatcggt | 120 |
| gttttggttt tgttgtggag aagatcctct gacagatcta gagaagttaa gcaattggct | 180 |
| gttccaaagc cagttactat cgttgaagaa gaagatgaat tcgaagttgc ttctggtaag | 240 |
| accagagttt ctattttcta cggtactcaa actggtactg ctgaaggttt tgctaaggct | 300 |
| ttggctgaag aaatcaaagc cagatacgaa aaagctgccg ttaaggttat tgatttggat | 360 |
| gattacacag ccgaagatga caaatacggt gaaaagttga agaaagaaac tatggccttc | 420 |
| ttcatgttgg ctacttatgg tgatggtgaa cctactgata atgctgctag attttacaag | 480 |
| tggttcaccg aaggtactga tagaggtgtt tggttggaac atttgagata cggtgtattc | 540 |
| ggtttgggta acagacaata cgaacacttc aacaagattg ccaaggttgt tgatgatttg | 600 |
| ttggttgaac aaggtgccaa gagattggtt actgttggtt tgggtgatga tgatcaatgc | 660 |
| atcgaagatg atttctccgc ttggaaagaa gccttgtggc cagaattgga tcaattattg | 720 |
| caagatgata ccaacaccgt ttctactcca tacactgctg ttattccaga atacagagtt | 780 |
| gttatccacg atccatctgt tacctcttat gaagatccat actctaacat ggctaacggt | 840 |
| aatgcctctt acgatattca tcatccatgt agagctaacg ttgccgtcca aaaagaattg | 900 |
| cataagccag aatctgacag aagttgcatc catttggaat tcgatatttt cgctactggt | 960 |
| ttgacttacg aaaccggtga tcatgttggt gtttacgctg ataattgtga tgatactgta | 1020 |
| gaagaagccg ctaagttgtt gggtcaacca ttggatttgt tgttctccat tcataccgat | 1080 |
| aacaacgacg gtacttcttt gggttcttct ttgccaccac catttccagg tccatgtact | 1140 |
| ttgagaactg ctttggctag atatgccgat ttgttgaatc caccaaaaaa ggctgctttg | 1200 |
| attgctttag ctgctcatgc tgatgaacca tctgaagctg aaagattgaa gttcttgtca | 1260 |
| tctccacaag gtaaggacga atattctaaa tgggttgtcg gttcccaaag atccttggtt | 1320 |
| gaagttatgg ctgaatttcc atctgctaaa ccaccattgg gtgtattttt tgctgctgtt | 1380 |
| gttcctagat tgcaacctag atattactcc atctcttcca gtccaagatt tgctccacat | 1440 |
| agagttcatg ttacttgcgc tttggtttat ggtccaactc caactggtag aattcacaga | 1500 |
| ggtgtatgtt cattctggat gaagaatgtt gtcccattgg aaaagtctca aaactgttct | 1560 |
| tgggccccaa ttttcatcag acaatctaat ttcaagttgc cagccgatca ttctgttcca | 1620 |
| atagttatgg ttggtccagg tactggttta gctcctttta gaggtttctt acaagaaaga | 1680 |
| ttggccttga aagaagaagg tgctcaagtt ggtcctgctt tgttgttttt tggttgcaga | 1740 |
| aacagacaaa tggacttcat ctacgaagtc gaattgaaca actttgtcga acaaggtgct | 1800 |
| ttgtccgaat tgatcgttgc tttttcaaga gaaggtccat ccaaagaata cgtccaacat | 1860 |
| aagatggttg aaaaggcagc ttacatgtgg aacttgattt ctcaaggtgg ttacttctac | 1920 |
| gtttgtggtg atgctaaagg tatggctaga gatgttcata gaacattgca taccatcgtc | 1980 |
| caacaagaag aaaaggttga ttctaccaag gccgaatcca tcgttaagaa attgcaaatg | 2040 |
| gacggtagat acttgagaga tgtttggtga | 2070 |

SEQ ID NO: 88
R. suavissimus

| | |
|---|---|
| MSSNSDLVRR LESVLGVSFG GSVTDSVVVI ATTSIALVIG VLVLLWRRSS DRSREVKQLA | 60 |
| VPKPVTIVEE EDEFEVASGK TRVSIFYGTQ TGTAEGFAKA LAEEIKARYE KAAVKVIDLD | 120 |
| DYTAEDDKYG EKLKKETMAF FMLATYGDGE PTDNAARFYK WFTEGTDRGV WLEHLRYGVF | 180 |
| GLGNRQYEHF NKIAKVVDDL LVEQGAKRLV TVGLGDDDQC IEDDFSAWKE ALWPELDQLL | 240 |
| QDDTNTVSTP YTAVIPEYRV VIHDPSVTSY EDPYSNMANG NASYDIHHPC RANVAVQKEL | 300 |
| HKPESDRSCI HLEFDIFATG LTYETGDHVG VYADNCDDTV EEAAKLLGQP LDLLFSIHTD | 360 |
| NNDGTSLGSS LPPPFPGPCT LRTALARYAD LLNPPKKAAL IALAAHADEP SEAERLKFLS | 420 |
| SPQGKDEYSK WVVGSQRSLV EVMAEFPSAK PPLGVFFAAV VPRLQPRYYS ISSSPRFAPH | 480 |
| RVHVTCALVY GPTPTGRIHR GVCSFWMKNV VPLEKSQNCS WAPIFIRQSN FKLPADHSVP | 540 |
| IVMVGPGTGL APFRGFLQER LALKEEGAQV GPALLFFGCR NRQMDFIYEV ELNNFVEQGA | 600 |
| LSELIVAFSR EGPSKEYVQH KMVEKAAYMW NLISQGGYFY VCGDAKGMAR DVHRTLHTIV | 660 |
| QQEEKVDSTK AESIVKKLQM DGRYLRDVW | 689 |

SEQ ID NO: 89
Artificial Sequence

| | |
|---|---|
| atgacttctg cactttatgc ctccgatctt ttcaaacaat tgaaaagtat catgggaacg | 60 |
| gattctttgt ccgatgatgt tgtattagtt attgctacaa cttctctggc actggttgct | 120 |
| ggtttcgttg tcttattgtg gaaaaagacc acggcagatc gttccggcga gctaaagcca | 180 |
| ctaatgatcc ctaagtctct gatggcgaaa gatgaggatg atgacttaga tctaggttct | 240 |
| ggaaaaacga gagtctctat cttcttcggc acacaaaccg gaacagccga aggattcgct | 300 |
| aaagcacttt cagaagagat caaagcaaga tacgaaaagg cggctgtaaa agtaatcgat | 360 |
| ttggatgatt acgctgccga tgatgaccaa tatgaggaaa agttgaaaaa ggaaacattg | 420 |
| gctttctttt gtgtagccac gtatggtgat ggtgaaccaa ccgataacgc cgcaagattc | 480 |
| tacaagtggt ttactgaaga gaacgaaaga gatatcaagt tgcagcaact tgcttacggc | 540 |
| gtttttgcct taggtaacag acaatacgag cactttaaca agataggtat tgtcttagat | 600 |
| gaagagttat gcaaaaaggg tgcgaagaga ttgattgaag tcggtttagg agatgatgat | 660 |
| caatctatcg aggatgactt taatgcatgg aaggaatctt tgtggtctga attagataag | 720 |
| ttacttaagg acgaagatga taaatccgtt gccactccat acacagccgt cattccagaa | 780 |
| tatagagtag ttactcatga tccaagattc acaacacaga aatcaatgga aagtaatgtg | 840 |
| gctaatggta atactaccat cgatattcat catccatgta gagtagacgt tgcagttcaa | 900 |
| aaggaattgc acactcatga atcagacaga tcttgcatac atcttgaatt tgatatatca | 960 |
| cgtactggta tcacttacga aacaggtgat cacgtgggtg tctacgctga aaaccatgtt | 1020 |
| gaaattgtag aggaagctgg aaagttgttg ggccatagtt tagatcttgt tttctcaatt | 1080 |
| catgccgata aagaggatgg ctcaccacta gaaagtgcag tgcctccacc atttccagga | 1140 |
| ccatgcaccc taggtaccgg tttagctcgt tacgcggatc tgttaaatcc tccacgtaaa | 1200 |
| tcagctctag tggccttggc tgcgtacgcc acagaacctt ctgaggcaga aaaactgaaa | 1260 |
| catctaactt caccagatgg taaggatgaa tactcacaat ggatagtagc tagtcaacgt | 1320 |
| tctttactag aagttatggc tgctttccca tccgctaaac ctcctttggg tgttttcttc | 1380 |
| gccgcaatag cgcctagact gcaaccaaga tactattcaa tttcatcctc acctagactg | 1440 |
| gcaccatcaa gagttcatgt cacatccgct ttagtgtacg gtccaactcc tactggtaga | 1500 |
| atccataagg gcgtttgttc aacatggatg aaaaacgcgg ttccagcaga gaagtctcac | 1560 |
| gaatgttctg gtgctccaat ctttatcaga gcctccaact tcaaactgcc ttccaatcct | 1620 |
| tctactccta ttgtcatggt cggtcctggt acaggtcttg ctccattcag aggtttctta | 1680 |
| caagagagaa tggccttaaa ggaggatggt gaagagttgg gatcttcttt gttgtttttc | 1740 |
| ggctgtagaa acagacaaat ggatttcatc tacgaagatg aactgaataa ctttgtagat | 1800 |
| caaggagtta tttcagagtt gataatggct ttttctagag aaggtgctca gaaggagtac | 1860 |
| gtccaacaca aaatgatgga aaaggccgca caagtttggg acttaatcaa agaggaaggc | 1920 |
| tatctatatg tctgtggtga tgcaaagggt atggcaagag atgttcacag aacacttcat | 1980 |
| actatagtcc aggaacagga aggcgttagt tcttctgaag cggaagcaat tgtgaaaaag | 2040 |
| ttacaaacag agggaagata cttgagagat gtgtggtaa | 2079 |

SEQ ID NO: 90
A. thaliana

| | |
|---|---|
| MTSALYASDL FKQLKSIMGT DSLSDDVVLV IATTSLALVA GFVVLLWKKT TADRSGELKP | 60 |
| LMIPKSLMAK DEDDDLDLGS GKTRVSIFFG TQTGTAEGFA KALSEEIKAR YEKAAVKVID | 120 |
| LDDYAADDDQ YEEKLKKETL AFFCVATYGD GEPTDNAARF YKWFTEENER DIKLQQLAYG | 180 |
| VFALGNRQYE HFNKIGIVLD EELCKKGAKR LIEVGLGDDD QSIEDDFNAW KESLWSELDK | 240 |
| LLKDEDDKSV ATPYTAVIPE YRVVTHDPRF TTQKSMESNV ANGNTTIDIH HPCRVDVAVQ | 300 |
| KELHTHESDR SCIHLEFDIS RTGITYETGD HVGVYAENHV EIVEEAGKLL GHSLDLVFSI | 360 |
| HADKEDGSPL ESAVPPPFPG PCTLGTGLAR YADLLNPPRK SALVALAAYA TEPSEAEKLK | 420 |
| HLTSPDGKDE YSQWIVASQR SLLEVMAAFP SAKPPLGVFF AAIAPRLQPR YYSISSSPRL | 480 |
| APSRVHVTSA LVYGPTPTGR IHKGVCSTWM KNAVPAEKSH ECSGAPIFIR ASNFKLPSNP | 540 |
| STPIVMVGPG TGLAPFRGFL QERMALKEDG EELGSSLLFF GCRNRQMDFI YEDELNNFVD | 600 |
| QGVISELIMA FSREGAQKEY VQHKMMEKAA QVWDLIKEEG YLYVCGDAKG MARDVHRTLH | 660 |
| TIVQEQEGVS SSEAEAIVKK LQTEGRYLRD VW | 692 |

SEQ ID NO: 91
Artificial Sequence

| | |
|---|---|
| atgtcttcct cttcctcttc cagtacctct atgattgatt tgatggctgc tattattaaa | 60 |
| ggtgaaccag ttatcgtctc cgacccagca aatgcctctg cttatgaatc agttgctgca | 120 |
| gaattgtctt caatgttgat cgaaaacaga caattcgcca tgatcgtaac tacatcaatc | 180 |
| gctgttttga tcggttgtat tgtcatgttg gtatggagaa gatccggtag tggtaattct | 240 |
| aaaagagtcg aacctttgaa accattagta attaagccaa gagaagaaga aatagatgac | 300 |
| ggtagaaaga aagttacaat atttttcggt acccaaactg gtacagctga aggttttgca | 360 |
| aaagccttag gtgaagaagc taaggcaaga tacgaaaaga ctagattcaa gatagtcgat | 420 |
| ttggatgact atgccgctga tgacgatgaa tacgaagaaa agttgaagaa agaagatgtt | 480 |
| gcatttttct ttttggcaac ctatggtgac ggtgaaccaa ctgacaatgc agccagattc | 540 |
| tacaaatggt ttacagaggg taatgatcgt ggtgaatggt tgaaaaactt aaagtacggt | 600 |
| gttttcggtt tgggtaacag acaatacgaa catttcaaca aagttgcaaa ggttgtcgac | 660 |
| gatattttgg tcgaacaagg tgctcaaaga ttagtccaag taggtttggg tgacgatgac | 720 |
| caatgtatag aagatgactt tactgcctgg agagaagctt tgtggcctga attagacaca | 780 |
| atcttgagag aagaaggtga caccgccgtt gctaccccat atactgctgc agtattagaa | 840 |
| tacagagttt ccatccatga tagtgaagac gcaaagttta atgatatcac tttggccaat | 900 |
| ggtaacggtt atacagtttt cgatgcacaa cacccttaca aagctaacgt tgcagtcaag | 960 |
| agagaattac atacaccaga atccgacaga agttgtatac acttggaatt tgatatcgct | 1020 |
| ggttccggtt taaccatgaa gttgggtgac catgtaggtg ttttatgcga caatttgtct | 1080 |
| gaaactgttg atgaagcatt gagattgttg gatatgtccc ctgacactta ttttagtttg | 1140 |
| cacgctgaaa aagaagatgg tacaccaatt tccagttctt taccacctcc attccctcca | 1200 |
| tgtaacttaa gaacagcctt gaccagatac gcttgcttgt tatcatcccc taaaaagtcc | 1260 |
| gccttggttg ctttagccgc tcatgctagt gatcctactg aagcagaaag attgaaacac | 1320 |
| ttagcatctc cagccggtaa agatgaatat tcaaagtggg tagttgaatc tcaaagatca | 1380 |
| ttgttagaag ttatggcaga atttccatct gccaagcctc cattaggtgt cttctttgct | 1440 |
| ggtgtagcac ctagattgca accaagattc tactcaatca gttcttcacc taagatcgct | 1500 |
| gaaactagaa ttcatgttac atgtgcatta gtctacgaaa agatgccaac cggtagaatt | 1560 |
| cacaagggtg tatgctctac ttggatgaaa aatgctgttc cttacgaaaa atcagaaaag | 1620 |
| ttgttcttag gtagaccaat cttcgtaaga caatcaaact tcaagttgcc ttctgattca | 1680 |
| aaggttccaa taatcatgat aggtcctggt acaggtttag ccccattcag aggtttcttg | 1740 |
| caagaaagat tggctttagt tgaatctggt gtcgaattag gtccttcagt tttgttcttt | 1800 |
| ggttgtagaa acagaagaat ggatttcatc tatgaagaag aattgcaaag attcgtcgaa | 1860 |
| tctggtgcat tggccgaatt atctgtagct ttttcaagag aaggtccaac taaggaatac | 1920 |
| gttcaacata agatgatgga taaggcatcc gacatatgga acatgatcag tcaaggtgct | 1980 |
| tatttgtacg tttgcggtga cgcaaagggt atggccagag atgtccatag atctttgcac | 2040 |
| acaattgctc aagaacaagg ttccatggat agtaccaaag ctgaaggttt cgtaaagaac | 2100 |
| ttacaaactt ccggtagata cttgagagat gtctggtga | 2139 |

SEQ ID NO: 92
A. thaliana

| | |
|---|---|
| MSSSSSSSTS MIDLMAAIIK GEPVIVSDPA NASAYESVAA ELSSMLIENR QFAMIVTTSI | 60 |
| AVLIGCIVML VWRRSGSGNS KRVEPLKPLV IKPREEEIDD GRKKVTIFFG TQTGTAEGFA | 120 |
| KALGEEAKAR YEKTRFKIVD LDDYAADDDE YEEKLKKEDV AFFFLATYGD GEPTDNAARF | 180 |
| YKWFTEGNDR GEWLKNLKYG VFGLGNRQYE HFNKVAKVVD DILVEQGAQR LVQVGLGDDD | 240 |
| QCIEDDFTAW REALWPELDT ILREEGDTAV ATPYTAAVLE YRVSIHDSED AKFNDITLAN | 300 |
| GNGYTVFDAQ HPYKANVAVK RELHTPESDR SCIHLEFDIA GSGLTMKLGD HVGVLCDNLS | 360 |
| ETVDEALRLL DMSPDTYFSL HAEKEDGTPI SSSLPPPFPP CNLRTALTRY ACLLSSPKKS | 420 |
| ALVALAAHAS DPTEAERLKH LASPAGKDEY SKWVVESQRS LLEVMAEFPS AKPPLGVFFA | 480 |
| GVAPRLQPRF YSISSSPKIA ETRIHVTCAL VYEKMPTGRI HKGVCSTWMK NAVPYEKSEK | 540 |
| LFLGRPIFVR QSNFKLPSDS KVPIIMIGPG TGLAPFRGFL QERLALVESG VELGPSVLFF | 600 |
| GCRNRRMDFI YEEELQRFVE SGALAELSVA FSREGPTKEY VQHKMMDKAS DIWNMISQGA | 660 |
| YLYVCGDAKG MARDVHRSLH TIAQEQGSMD STKAEGFVKN LQTSGRYLRD VW | 712 |

SEQ ID NO: 93
Artificial Sequence

| | |
|---|---|
| atggaagcct cttacctata catttctatt ttgcttttac tggcatcata cctgttcacc | 60 |
| actcaactta gaaggaagag cgctaatcta ccaccaaccg tgtttccatc aataccaatc | 120 |
| attggacact tatacttact caaaaagcct ctttatagaa ctttagcaaa aattgccgct | 180 |
| aagtacggac caatactgca attacaactc ggctacagac gtgttctggt gatttcctca | 240 |
| ccatcagcag cagaagagtg ctttaccaat aacgatgtaa tcttcgcaaa tagacctaag | 300 |
| acattgtttg gcaaaatagt gggtggaaca tcccttggca gtttatccta cggcgatcaa | 360 |
| tggcgtaatc taaggagagt agcttctatc gaaatcctat cagttcatag gttgaacgaa | 420 |
| tttcatgata tcagagtgga tgagaacaga ttgttaatta gaaaacttag aagttcatct | 480 |
| tctcctgtta ctcttataac agtcttttat gctctaacat tgaacgtcat tatgagaatg | 540 |
| atctctggca aaagatattt cgacagtggg gatagagaat tggaggagga aggtaagaga | 600 |
| tttcgagaaa tcttagacga aacgttgctt ctagccggtg cttctaatgt tggcgactac | 660 |
| ttaccaatat tgaactggtt gggagttaag tctcttgaaa agaaattgat cgctttgcag | 720 |
| aaaaagagag atgacttttt ccagggtttg attgaacagg ttagaaaatc tcgtggtgct | 780 |
| aaagtaggca aaggtagaaa aacgatgatc gaactcttat tatctttgca agagtcagaa | 840 |
| cctgagtact atacagatgc tatgataaga tcttttgtcc taggtctgct ggctgcaggt | 900 |
| agtgatactt cagcgggcac tatggaatgg gccatgagct tactggtcaa tcacccacat | 960 |
| gtattgaaga aagctcaagc tgaaatcgat agagttatcg gtaataacag attgattgac | 1020 |
| gagtcagaca ttggaaatat cccttacatc gggtgtatta tcaatgaaac tctaagactc | 1080 |
| tatccagcag ggccattgtt gttcccacat gaaagttctg ccgactgcgt tatttccggt | 1140 |
| tacaatatac ctagaggtac aatgttaatc gtaaaccaat gggcgattca tcacgatcct | 1200 |
| aaagtctggg atgatcctga aacctttaaa cctgaaagat ttcaaggatt agaaggaact | 1260 |
| agagatggtt tcaaacttat gccattcggt tctgggagaa gaggatgtcc aggtgaaggt | 1320 |
| ttggcaataa ggctgttagg gatgacacta ggctcagtga tccaatgttt tgattgggag | 1380 |
| agagtaggag atgagatggt tgacatgaca gaaggtttgg gtgtcacact tcctaaggcc | 1440 |
| gttccattag ttgccaaatg taagccacgt tccgaaatga ctaatctcct atccgaactt | 1500 |
| taa | 1503 |
| SEQ ID NO: 94 | |
| S. rebaudiana | |
| MEASYLYISI LLLLASYLFT TQLRRKSANL PPTVFPSIPI IGHLYLLKKP LYRTLAKIAA | 60 |
| KYGPILQLQL GYRRVLVISS PSAAEECFTN NDVIFANRPK TLFGKIVGGT SLGSLSYGDQ | 120 |
| WRNLRRVASI EILSVHRLNE FHDIRVDENR LLIRKLRSSS SPVTLITVFY ALTLNVIMRM | 180 |
| ISGKRYFDSG DRELEEEGKR FREILDETLL LAGASNVGDY LPILNWLGVK SLEKKLIALQ | 240 |
| KKRDDFFQGL IEQVRKSRGA KVGKGRKTMI ELLLSLQESE PEYYTDAMIR SFVLGLLAAG | 300 |
| SDTSAGTMEW AMSLLVNHPH VLKKAQAEID RVIGNNRLID ESDIGNIPYI GCIINETLRL | 360 |
| YPAGPLLFPH ESSADCVISG YNIPRGTMLI VNQWAIHHDP KVWDDPETFK PERFQGLEGT | 420 |
| RDGFKLMPFG SGRRGCPGEG LAIRLLGMTL GSVIQCFDWE RVGDEMVDMT EGLGVTLPKA | 480 |
| VPLVAKCKPR SEMTNLLSEL | 500 |

SEQ ID NO: 95
R. suavissimus

| | |
|---|---|
| atggaagtaa cagtagctag tagtgtagcc ctgagcctgg tctttattag catagtagta | 60 |
| agatgggcat ggagtgtggt gaattgggtg tggtttaagc cgaagaagct ggaaagattt | 120 |
| ttgagggagc aaggccttaa aggcaattcc tacaggtttt tatatggaga catgaaggag | 180 |
| aactctatcc tgctcaaaca agcaagatcc aaacccatga acctctccac ctcccatgac | 240 |
| atagcacctc aagtcacccc ttttgtcgac caaaccgtga aagcttacgg taagaactct | 300 |
| tttaattggg ttggccccat accaagggtg aacataatga atccagaaga tttgaaggac | 360 |
| gtcttaacaa aaaatgttga ctttgttaag ccaatatcaa acccacttat caagttgcta | 420 |
| gctacaggta ttgcaatcta tgaaggtgag aaatggacta aacacagaag gattatcaac | 480 |
| ccaacattcc attcggagag gctaaagcgt atgttacctt catttcacca aagttgtaat | 540 |
| gagatggtca aggaatggga gagcttggtg tcaaaagagg gttcatcatg tgagttggat | 600 |
| gtctggcctt ttcttgaaaa tatgtcggca gatgtgatct cgagaacagc atttggaact | 660 |
| agctacaaaa aaggacagaa aatctttgaa ctcttgagag agcaagtaat atatgtaacg | 720 |
| aaaggctttc aaagttttta cattccagga tggaggtttc tcccaactaa gatgaacaag | 780 |
| aggatgaatg agattaacga agaaataaaa ggattaatca ggggtattat aattgacaga | 840 |
| gagcaaatca ttaaggcagg tgaagaaacc aacgatgact tattaggtgc acttatggag | 900 |
| tcaaacttga aggacattcg ggaacatggg aaaaacaaca aaaatgttgg gatgagtatt | 960 |
| gaagatgtaa ttcaggagtg taagctgttt tactttgctg ggcaagaaac cacttcagtg | 1020 |
| ttgctggctt ggacaatggt tttacttggt caaaatcaga actggcaaga tcgagcaaga | 1080 |
| caagaggttt tgcaagtctt tggaagcagc aagccagatt ttgatggtct agctcacctt | 1140 |
| aaagtcgtaa ccatgatttt gcttgaagtt cttcgattat acccaccagt cattgaactt | 1200 |
| attcgaacca ttcacaagaa aacacaactt gggaagctct cactaccaga aggagttgaa | 1260 |
| gtccgcttac caacactgct cattcaccat gacaaggaac tgtggggtga tgatgcaaac | 1320 |
| cagttcaatc cagagaggtt ttcggaagga gtttccaaag caacaaagaa ccgactctca | 1380 |
| ttcttcccct tcggagccgg tccacgcatt tgcattggac agaacttttc tatgatggaa | 1440 |
| gcaaagttgg ccttagcatt gatcttgcaa cacttcacct ttgagctttc tccatctcat | 1500 |
| gcacatgctc cttcccatcg tataaccctt caaccacagt atggtgttcg tatcatttta | 1560 |
| catcgacgtt ag | 1572 |

SEQ ID NO: 96
Artificial Sequence

| | |
|---|---|
| atggaagtca ctgtcgcctc ttctgtcgct ttatccttag tcttcatttc cattgtcgtc | 60 |
| agatgggctt ggtccgttgt caactgggtt tggttcaaac caaagaagtt ggaaagattc | 120 |
| ttgagagagc aaggtttgaa gggtaattct tatagattct tgtacggtga catgaaggaa | 180 |
| aattctattt tgttgaagca agccagatcc aaaccaatga acttgtctac ctctcatgat | 240 |
| attgctccac aagttactcc attcgtcgat caaactgtta aagcctacgg taagaactct | 300 |
| ttcaattggg ttggtccaat tcctagagtt aacatcatga acccagaaga tttgaaggat | 360 |
| gtcttgacca agaacgttga cttcgttaag ccaatttcca acccattgat taaattgttg | 420 |
| gctactggta ttgccattta cgaaggtgaa aagtggacta agcatagaag aatcatcaac | 480 |
| cctaccttcc actctgaaag attgaagaga atgttaccat ctttccatca atcctgtaat | 540 |
| gaaatggtta aggaatggga atccttggtt tctaaagaag gttcttcttg cgaattggat | 600 |
| gtttggccat tcttggaaaa tatgtctgct gatgtcattt ccagaaccgc tttcggtacc | 660 |
| tcctacaaga agggtcaaaa gattttcgaa ttgttgagag agcaagttat ttacgttacc | 720 |
| aagggtttcc aatccttcta catcccaggt tggagattct tgccaactaa aatgaacaag | 780 |
| cgtatgaacg agatcaacga agaaattaaa ggtttgatca gaggtattat tatcgacaga | 840 |
| gaacaaatta ttaaagctgg tgaagaaacc aacgatgatt tgttgggtgc tttgatggag | 900 |
| tccaacttga aggatattag agaacatggt aagaacaaca agaatgttgg tatgtctatt | 960 |
| gaagatgtta ttcaagaatg taagttattc tacttcgctg gtcaagagac cacttctgtt | 1020 |
| ttgttagcct ggactatggt cttgttaggt caaaaccaaa attggcaaga tagagctaga | 1080 |
| caagaagttt tgcaagtctt cggttcttcc aagccagact ttgatggttt ggcccacttg | 1140 |
| aaggttgtta ctatgatttt gttagaagtt ttgagattgt acccaccagt cattgagtta | 1200 |
| atcagaacca ttcataaaaa gactcaattg ggtaaattat ctttgccaga aggtgttgaa | 1260 |
| gtcagattac caaccttgtt gattcaccac gataaggaat tatggggtga cgacgctaat | 1320 |
| caatttaatc cagaaagatt ttccgaaggt gtttccaagg ctaccaaaaa ccgtttgtcc | 1380 |
| ttcttcccat ttggtgctgg tccacgtatt tgtatcggtc aaaacttttc catgatggaa | 1440 |
| gccaagttgg ctttggcttt aatcttgcaa cacttcactt tcgaattgtc tccatcccat | 1500 |
| gcccacgctc cttctcatag aatcacttta caaccacaat acggtgtcag aatcatctta | 1560 |
| cacagaagat aa | 1572 |

SEQ ID NO: 97
R. suavissimus

| | |
|---|---|
| MEVTVASSVA LSLVFISIVV RWAWSVVNWV WFKPKKLERF LREQGLKGNS YRFLYGDMKE | 60 |
| NSILLKQARS KPMNLSTSHD IAPQVTPFVD QTVKAYGKNS FNWVGPIPRV NIMNPEDLKD | 120 |
| VLTKNVDFVK PISNPLIKLL ATGIAIYEGE KWTKHRRIIN PTFHSERLKR MLPSFHQSCN | 180 |
| EMVKEWESLV SKEGSSCELD VWPFLENMSA DVISRTAFGT SYKKGQKIFE LLREQVIYVT | 240 |
| KGFQSFYIPG WRFLPTKMNK RMNEINEEIK GLIRGIIIDR EQIIKAGEET NDDLLGALME | 300 |
| SNLKDIREHG KNNKNVGMSI EDVIQECKLF YFAGQETTSV LLAWTMVLLG QNQNWQDRAR | 360 |
| QEVLQVFGSS KPDFDGLAHL KVVTMILLEV LRLYPPVIEL IRTIHKKTQL GKLSLPEGVE | 420 |
| VRLPTLLIHH DKELWGDDAN QFNPERFSEG VSKATKNRLS FFPFGAGPRI CIGQNFSMME | 480 |
| AKLALALILQ HFTFELSPSH AHAPSHRITL QPQYGVRIIL HRR | 523 |

SEQ ID NO: 98
P. avium

| | |
|---|---|
| atggaagcat caagggctag ttgtgttgcg ctatgtgttg tttgggtgag catagtaatt | 60 |
| acattggcat ggagggtgct gaattgggtg tggttgaggc caaagaaact agaaagatgc | 120 |
| ttgagggagc aaggccttac aggcaattct tacaggcttt tgtttggaga caccaaggat | 180 |
| ctctcgaaga tgctggaaca aacacaatcc aaacccatca aactctccac ctcccatgat | 240 |
| atagcgccac gagtcacccc atttttccat cgaactgtga actctaatgg caagaattct | 300 |
| tttgtttgga tgggccctat accaagagtg cacatcatga atccagaaga tttgaaagat | 360 |
| gccttcaaca gacatgatga ttttcataag acagtaaaaa atcctatcat gaagtctcca | 420 |
| ccaccgggca ttgtaggcat tgaaggtgag caatgggcta aacacagaaa gattatcaac | 480 |
| ccagcattcc atttagagaa gctaaagggt atggtaccaa tattttacca aagttgtagc | 540 |
| gagatgatta acaaatggga gagcttggtg tccaaagaga gttcatgtga gttggatgtg | 600 |
| tggccttatc ttgaaaattt taccagcgat gtgatttccc gagctgcatt tggaagtagc | 660 |
| tatgaagagg gaaggaaaat atttcaacta ctaagagagg aagcaaaagt ttattcggta | 720 |
| gctctacgaa gtgtttacat tccaggatgg aggtttctac caaccaagca gaacaagaag | 780 |
| acgaaggaaa ttcacaatga aattaaaggc ttacttaagg gcattataaa taaaagggaa | 840 |
| gaggcgatga aggcagggga agccactaaa gatgacttac taggaatact tatggagtcc | 900 |
| aacttcaggg aaattcagga acatgggaac aacaaaaatg ctggaatgag tattgaagat | 960 |
| gtaattggag agtgtaagtt gttttacttt gctgggcaag agaccacttc ggtgttgctt | 1020 |
| gtttggacaa tgattttact aagccaaaat caggattggc aagctcgtgc aagagaagag | 1080 |
| gtcttgaaag tctttggaag caacatccca acctatgaag agctaagtca cctaaaagtt | 1140 |
| gtgaccatga ttttacttga agttcttcga ttatacccat cagtcgttgc gcttcctcga | 1200 |
| accactcaca agaaaacaca gcttggaaaa ttatcattac cagctggagt ggaagtctcc | 1260 |
| ttgcccatac tgcttgttca ccatgacaaa gagttgtggg gtgaggatgc aaatgagttc | 1320 |
| aagccagaga ggttttcaga gggagtttca aaggcaacaa agaacaaatt tacatactta | 1380 |
| cctttcggag ggggtccaag gatttgcatt ggacaaaact ttgccatggt ggaagctaaa | 1440 |
| ttggccttgg ccctgatttt acaacacttt gcctttgagc tttctccatc ctatgctcat | 1500 |
| gctccttctg cagttataac ccttcaacct caatttggtg ctcatatcat tttgcataaa | 1560 |
| cgttga | 1566 |

SEQ ID NO: 99
Artificial Sequence

| | |
|---|---|
| atggaagctt ctagagcatc ttgtgttgct ttgtgtgttg tttgggtttc catcgttatt | 60 |
| actttggctt ggagagtttt gaattgggtc tggttaagac caaaaaagtt ggaaagatgc | 120 |
| ttgagagaac aaggtttgac tggtaactct tacagattgt tgttcggtga taccaaggac | 180 |
| ttgtctaaga tgttggaaca aactcaatcc aagcctatca agttgtctac ctctcatgat | 240 |
| attgctccaa gagttactcc attcttccat agaactgtta actccaacgg taagaactct | 300 |
| tttgtttgga tgggtccaat tccaagagtc catattatga accctgaaga tttgaaggac | 360 |
| gctttcaaca gacatgatga tttccataag accgtcaaga acccaattat gaagtctcca | 420 |
| ccaccaggta tagttggtat tgaaggtgaa caatgggcca aacatagaaa gattattaac | 480 |
| ccagccttcc acttggaaaa gttgaaaggt atggttccaa tcttctacca atcctgctct | 540 |
| gaaatgatta acaagtggga atccttggtt tccaaagaat cttcctgtga attggatgtc | 600 |
| tggccatatt tggaaaactt cacctccgat gttatttcca gagctgcttt tggttcttct | 660 |
| tacgaagaag gtagaaagat cttccaatta ttgagagaag aagccaaggt ttactccgtt | 720 |
| gctttgagat ctgtttacat tccaggttgg agattcttgc caactaagca aaacaaaaag | 780 |
| accaaagaaa tccacaacga aatcaagggt ttgttgaagg gtatcatcaa caagagagaa | 840 |
| gaagctatga aggctggtga agctacaaaa gatgatttgt tgggtatctt gatggaatcc | 900 |
| aacttcagag aaatccaaga acacggtaac aacaagaatg ccggtatgtc tattgaagat | 960 |
| gttatcggtg aatgcaagtt gttctacttt gctggtcaag aaactacctc cgttttgttg | 1020 |
| gtttggacca tgattttgtt gtcccaaaat caagattggc aagctagagc tagagaagaa | 1080 |
| gtcttgaaag ttttcggttc taacatccca acctacgaag aattgtctca cttgaaggtt | 1140 |
| gtcactatga tcttgttgga agtattgaga ttatacccat ccgttgttgc attgccaaga | 1200 |
| actactcata agaaaactca attgggtaaa ttgtccttgc cagctggtgt tgaagtttct | 1260 |
| ttgccaattt tgttagtcca ccacgacaaa gaattgtggg gtgaagatgc taatgaattc | 1320 |
| aagccagaaa gattctccga aggtgtttct aaagctacca agaacaagtt cacttacttg | 1380 |
| ccatttggtg gtggtccaag aatatgtatt ggtcaaaatt tcgctatggt cgaagctaaa | 1440 |
| ttggctttgg ctttgatctt gcaacatttc gctttcgaat tgtcaccatc ttatgctcat | 1500 |
| gctccatctg ctgttattac attgcaacca caatttggtg cccatatcat cttgcataag | 1560 |
| agataac | 1567 |

SEQ ID NO: 100
P. avium

| | |
|---|---|
| MEASRASCVA LCVVWVSIVI TLAWRVLNWV WLRPKKLERC LREQGLTGNS YRLLFGDTKD | 60 |
| LSKMLEQTQS KPIKLSTSHD IAPRVTPFFH RTVNSNGKNS FVWMGPIPRV HIMNPEDLKD | 120 |
| AFNRHDDFHK TVKNPIMKSP PPGIVGIEGE QWAKHRKIIN PAFHLEKLKG MVPIFYQSCS | 180 |
| EMINKWESLV SKESSCELDV WPYLENFTSD VISRAAFGSS YEEGRKIFQL LREEAKVYSV | 240 |
| ALRSVYIPGW RFLPTKQNKK TKEIHNEIKG LLKGIINKRE EAMKAGEATK DDLLGILMES | 300 |
| NFREIQEHGN NKNAGMSIED VIGECKLFYF AGQETTSVLL VWTMILLSQN QDWQARAREE | 360 |
| VLKVFGSNIP TYEELSHLKV VTMILLEVLR LYPSVVALPR TTHKKTQLGK LSLPAGVEVS | 420 |
| LPILLVHHDK ELWGEDANEF KPERFSEGVS KATKNKFTYL PFGGGPRICI GQNFAMVEAK | 480 |
| LALALILQHF AFELSPSYAH APSAVITLQP QFGAHIILHK R | 521 |

SEQ ID NO: 101
P. mume

| | |
|---|---|
| ASWVAVLSVV WVSMVIAWAW RVLNWVWLRP KKLEKCLREQ GLAGNSYRLL FGDTKDLSKM | 60 |
| LEQTQSKPIK LSTSHDIAPH VTPFFHQTVN SYGKNSFVWM GPIPRVHIMN PEDLKDTFNR | 120 |
| HDDFHKVVKN PIMKSLPQGI VGIEGEQWAK HRKIINPAFH LEKLKGMVPI FYRSCSEMIN | 180 |
| KWESLVSKES SCELDVWPYL ENFTSDVISR AAFGSSYEEG RKIFQLLREE AKIYTVAMRS | 240 |
| VYIPGWRFLP TKQNKKAKEI HNEIKGLLKG IINKREEAMK AGEATKDDLL GILMESNFRE | 300 |
| IQEHGNNKNA GMSIEDVIGE CKLFYFAGQE TTSVLLVWTM VLLSQNQDWQ ARAREEVLQV | 360 |
| FGSNIPTYEE LSQLKVVTMI LLEVLRLYPS VVALPRTTHK KTQLGKLSLP AGVEVSLPIL | 420 |
| LVHHDKELWG EDANEFKPER FSEGVSKATK NQFTYFPFGG GPRICIGQNF AMMEAKLALS | 480 |
| LILRHFALEL SPLYAHAPSV TITLQPQYGA HIILHKR | 517 |

SEQ ID NO: 102
P. mume

| | |
|---|---|
| MEASRPSCVA LSVVLVSIVI AWAWRVLNWV WLRPNKLERC LREQGLTGNS YRLLFGDTKE | 60 |
| ISMMVEQAQS KPIKLSTTHD IAPRVIPFSH QIVYTYGRNS FVWMGPTPRV TIMNPEDLKD | 120 |
| AFNKSDEFQR AISNPIVKSI SQGLSSLEGE KWAKHRKIIN PAFHLEKLKG MLPTFYQSCS | 180 |
| EMINKWESLV FKEGSREMDV WPYLENLTSD VISRAAFGSS YEEGRKIFQL LREEAKFYTI | 240 |
| AARSVYIPGW RFLPTKQNKR MKEIHKEVRG LLKGIINKRE DAIKAGEAAK GNLLGILMES | 300 |
| NFREIQEHGN NKNAGMSIED VIGECKLFYF AGQETTSVLL VWTLVLLSQN QDWQARAREE | 360 |
| VLQVFGTNIP TYDQLSHLKV VTMILLEVLR LYPAVVELPR TTYKKTQLGK FLLPAGVEVS | 420 |
| LHIMLAHHDK ELWGEDAKEF KPERFSEGVS KATKNQFTYF PFGAGPRICI GQNFAMLEAK | 480 |
| LALSLILQHF TFELSPSYAH APSVTITLHP QFGAHFILHK R | 521 |

SEQ ID NO: 103
P. mume

| | |
|---|---|
| CVALSVVLVS IVIAWAWRVL NWVWLRPNKL ERCLREQGLT GNSYRLLFGD TKEISMMVEQ | 60 |
| AQSKPIKLST THDIAPRVIP FSHQIVYTYG RNSFVWMGPT PRVTIMNPED LKDAFNKSDE | 120 |
| FQRAISNPIV KSISQGLSSL EGEKWAKHRK IINPAFHLEK LKGMLPTFYQ SCSEMINKWE | 180 |
| SLVFKEGSRE MDVWPYLENL TSDVISRAAF GSSYEEGRKI FQLLREEAKF YTIAARSVYI | 240 |
| PGWRFLPTKQ NKRMKEIHKE VRGLLKGIIN KREDAIKAGE AAKGNLLGIL MESNFREIQE | 300 |
| HGNNKNAGMS IEDVIGECKL FYFAGQETTS VLLVWTLVLL SQNQDWQARA REEVLQVFGT | 360 |
| NIPTYDQLSH LKVVTMILLE VLRLYPAVVE LPRTTYKKTQ LGKFLLPAGV EVSLHIMLAH | 420 |
| HDKELWGEDA KEFKPERFSE GVSKATKNQF TYFPFGAGPR ICIGQNFAML EAKLALSLIL | 480 |
| QHFTFELSPS YAHAPSVTIT LHPQFGAHFI LHKR | 514 |

SEQ ID NO: 104
P. persica

| | |
|---|---|
| MGPIPRVHIM NPEDLKDTFN RHDDFHKVVK NPIMKSLPQG IVGIEGDQWA KHRKIINPAF | 60 |
| HLEKLKGMVP IFYQSCSEMI NIWKSLVSKE SSCELDVWPY LENFTSDVIS RAAFGSSYEE | 120 |
| GRKIFQLLRE EAKVYTVAVR SVYIPGWRFL PTKQNKKTKE IHNEIKGLLK GIINKREEAM | 180 |
| KAGEATKDDL LGILMESNFR EIQEHGNNKN AGMSIEDVIG ECKLFYFAGQ ETTSVLLVWT | 240 |
| MVLLSQNQDW QARAREEVLQ VFGSNIPTYE ELSHLKVVTM ILLEVLRLYP SVVALPRTTH | 300 |
| KKTQLGKLSL PAGVEVSLPI LLVHHDKELW GEDANEFKPE RFSEGVSKAT KNQFTYFPFG | 360 |
| GGPRICIGQN FAMMEAKLAL SLILQHFTFE LSPQYSHAPS VTITLQPQYG AHLILHKR | 418 |

SEQ ID NO: 105
Artificial Sequence

| | |
|---|---|
| atgggtttgt tcccattaga ggattcctac gcgctggtct ttgaaggact agcaataaca | 60 |
| ctggctttgt actatctact gtctttcatc tacaaaacat ctaaaaagac atgtacacct | 120 |
| cctaaagcat ctggtgaaat cattccaatt acaggaatca tattgaatct gctatctggc | 180 |
| tcaagtggtc tacctattat cttagcactt gcctctttag cagacagatg tggtcctatt | 240 |
| ttcaccatta ggctgggtat taggagagtg ctagtagtat caaattggga aatcgctaag | 300 |
| gagattttca ctacccacga tttgatagtt tctaatagac caaaatactt agccgctaag | 360 |
| attcttggtt tcaattatgt ttcattctct ttcgctccat acggcccata ttgggtcgga | 420 |
| atcagaaaga ttattgctac aaaactaatg tcttcttcca gacttcagaa gttgcaattt | 480 |
| gtaagagttt ttgaactaga aaactctatg aaatctatca gagaatcatg gaaggagaaa | 540 |
| aaggatgaag agggaaaggt attagttgag atgaaaaagt ggttctggga actgaatatg | 600 |
| aacatagtgt taaggacagt tgctggtaaa caatacactg gtacagttga tgatgccgat | 660 |
| gcaaagcgta tctccgagtt attcagagaa tggtttcact acactggcag atttgtcgtt | 720 |
| ggagacgctt ttccttttct aggttggttg gacctgggcg gatacaaaaa gacaatggaa | 780 |
| ttagttgcta gtagattgga ctcaatggtc agtaaatggt tagatgagca tcgtaaaaag | 840 |
| caagctaacg atgacaaaaa ggaggatatg gatttcatgg atatcatgat ctccatgaca | 900 |
| gaagcaaatt caccacttga aggatacggc actgatacta ttatcaagac cacatgtatg | 960 |
| actttgattg tttcaggagt tgatacaacc tcaatcgtac ttacttgggc cttatcactt | 1020 |
| ttgttaaaca acagagatac tttgaaaaag gcacaagagg aattagatat gtgcgtaggt | 1080 |
| aaaggaagac aagtcaacga gtctgatctt gttaacttga tatacttgga agcagtgctt | 1140 |
| aaagaggctt taagacttta cccagcagcg ttcttaggcg gaccaagagc attcttggaa | 1200 |
| gattgtactg ttgctggtta tagaattcca aagggcacct gcttgttgat taacatgtgg | 1260 |
| aaactgcata gagatccaaa catttggagt gatccttgcg aattcaagcc agaaagattt | 1320 |
| ttgacaccta atcaaaagga tgttgatgtg atcggtatgg atttcgaatt gataccattt | 1380 |
| ggtgccggca gaagatattg tccaggtact agattggctt tacagatgtt gcatatcgta | 1440 |
| ttagcgacat tgctgcaaaa cttcgaaatg tcaacaccaa acgatgcgcc agtcgatatg | 1500 |
| actgcttctg ttggcatgac aaatgccaaa gcatcacctt tagaagtctt gctatcacct | 1560 |
| cgtgttaaat ggtcctaa | 1578 |

SEQ ID NO: 106
S. rebaudiana

| | |
|---|---|
| MGLFPLEDSY ALVFEGLAIT LALYYLLSFI YKTSKKTCTP PKASGEHPIT GHLNLLSGSS | 60 |
| GLPHLALASL ADRCGPIFTI RLGIRRVLVV SNWEIAKEIF TTHDLIVSNR PKYLAAKILG | 120 |
| FNYVSFSFAP YGPYWVGIRK IIATKLMSSS RLQKLQFVRV FELENSMKSI RESWKEKKDE | 180 |
| EGKVLVEMKK WFWELNMNIV LRTVAGKQYT GTVDDADAKR ISELFREWFH YTGRFVVGDA | 240 |
| FPFLGWLDLG GYKKTMELVA SRLDSMVSKW LDEHRKKQAN DDKKEDMDFM DIMISMTEAN | 300 |
| SPLEGYGTDT IIKTTCMTLI VSGVDTTSIV LTWALSLLLN NRDTLKKAQE ELDMCVGKGR | 360 |
| QVNESDLVNL IYLEAVLKEA LRLYPAAFLG GPRAFLEDCT VAGYRIPKGT CLLINMWKLH | 420 |
| RDPNIWSDPC EFKPERFLTP NQKDVDVIGM DFELIPFGAG RRYCPGTRLA LQMLHIVLAT | 480 |
| LLQNFEMSTP NDAPVDMTAS VGMTNAKASP LEVLLSPRVK WS | 522 |

SEQ ID NO: 107
Artificial Sequence

| | |
|---|---|
| atgatacaag ttttaactcc aattctactc ttcctcatct tcttcgtttt ctggaaagtc | 60 |
| tacaaacatc aaaagactaa aatcaatcta ccaccaggtt ccttcggctg gccatttttg | 120 |
| ggtgaaacct tagccttact tagagcaggc tgggattctg agccagaaag attcgtaaga | 180 |
| gagcgtatca aaaagcatgg atctccactt gttttcaaga catcactatt tggagacaga | 240 |
| ttcgctgttc tttgcggtcc agctggtaat aagtttttgt tctgcaacga aaacaaatta | 300 |
| gtggcatctt ggtggccagt ccctgtaagg aagttgttcg gtaaaagttt actcacaata | 360 |
| agaggagatg aagcaaaatg gatgagaaaa atgctattgt cttacttggg tccagatgca | 420 |
| tttgccacac attatgccgt tactatggat gttgtaacac gtagacatat tgatgtccat | 480 |
| tggaggggca aggaggaagt taatgtattt caaacagtta agttgtacgc attcgaatta | 540 |
| gcttgtagat tattcatgaa cctagatgac ccaaaccaca tcgcgaaact cggtagtctt | 600 |
| ttcaacattt tcctcaaagg gatcatcgag cttcctatag acgttcctgg aactagattt | 660 |
| tactccagta aaaaggccgc agctgccatt agaattgaat tgaaaaagct cattaaagct | 720 |
| agaaaactcg aattgaagga gggtaaggcg tcttcttcac aggacttgct ttctcatcta | 780 |
| ttaacatcac ctgatgagaa tgggatgttc ttgacagaag aggaaatagt cgataacatt | 840 |
| ctacttttgt tattcgctgg tcacgatacc tctgcactat caataacact tttgatgaaa | 900 |
| accttaggtg aacacagtga tgtgtacgac aaggttttga aggaacaatt agaaatttcc | 960 |
| aaaacaaagg aggcttggga atcactaaag tgggaagata tccagaagat gaagtactca | 1020 |
| tggtcagtaa tctgtgaagt catgagattg aatcctcctg tcatagggac atacagagag | 1080 |
| gcgttggttg atatcgacta tgctggttac actatcccaa aaggatggaa gttgcattgg | 1140 |
| tcagctgttt ctactcaaag agacgaagcc aatttcgaag atgtaactag attcgatcca | 1200 |
| tccagatttg aaggggcagg ccctactcca ttcacatttg tgcctttcgg tggaggtcct | 1260 |
| agaatgtgtt taggcaaaga gtttgccagg ttagaagtgt tagcatttct ccacaacatt | 1320 |
| gttaccaact ttaagtggga tcttctaatc cctgatgaga agatcgaata tgatccaatg | 1380 |
| gctactccag ctaagggctt gccaattaga cttcatccac accaagtcta a | 1431 |

SEQ ID NO: 108
S. rebaudiana

| | |
|---|---|
| MIQVLTPILL FLIFFVFWKV YKHQKTKINL PPGSFGWPFL GETLALLRAG WDSEPERFVR | 60 |
| ERIKKHGSPL VFKTSLFGDR FAVLCGPAGN KFLFCNENKL VASWWPVPVR KLFGKSLLTI | 120 |
| RGDEAKWMRK MLLSYLGPDA FATHYAVTMD VVTRRHIDVH WRGKEEVNVF QTVKLYAFEL | 180 |
| ACRLFMNLDD PNHIAKLGSL FNIFLKGIIE LPIDVPGTRF YSSKKAAAAI RIELKKLIKA | 240 |
| RKLELKEGKA SSSQDLLSHL LTSPDENGMF LTEEEIVDNI LLLLFAGHDT SALSITLLMK | 300 |
| TLGEHSDVYD KVLKEQLEIS KTKEAWESLK WEDIQKMKYS WSVICEVMRL NPPVIGTYRE | 360 |
| ALVDIDYAGY TIPKGWKLHW SAVSTQRDEA NFEDVTRFDP SRFEGAGPTP FTFVPFGGGP | 420 |
| RMCLGKEFAR LEVLAFLHNI VTNFKWDLLI PDEKIEYDPM ATPAKGLPIR LHPHQV | 476 |

SEQ ID NO: 109
Artificial Sequence

| | |
|---|---|
| atggagtctt tagtggttca tacagtaaat gctatctggt gtattgtaat cgtcgggatt | 60 |
| ttctcagttg gttatcacgt ttacggtaga gctgtggtcg aacaatggag aatgagaaga | 120 |
| tcactgaagc tacaaggtgt taaaggccca ccaccatcca tcttcaatgg taacgtctca | 180 |
| gaaatgcaac gtatccaatc cgaagctaaa cactgctctg gcgataacat tatctcacat | 240 |
| gattattctt cttcattatt cccacacttc gatcactgga gaaaacagta cggcagaatc | 300 |
| tacacatact ctactggatt aaagcaacac ttgtacatca atcatccaga aatggtgaag | 360 |
| gagctatctc agactaacac attgaacttg ggtagaatca cccatataac caaaagattg | 420 |
| aatcctatct taggtaacgg aatcataacc tctaatggtc ctcattgggc ccatcagcgt | 480 |
| agaattatcg cctacgagtt tactcatgat aagatcaagg gtatggttgg tttgatggtt | 540 |
| gagtctgcta tgcctatgtt gaataagtgg gaggagatgg taaagagagg cggagaaatg | 600 |
| ggatgcgaca taagagttga tgaggacttg aaagatgttt cagcagatgt gattgcaaaa | 660 |
| gcctgtttcg gatcctcatt ttctaaaggt aaggctattt tctctatgat aagagatttg | 720 |
| cttacagcta tcacaaagag aagtgttcta ttcagattca acggattcac tgatatggtc | 780 |
| tttgggagta aaaagcatgg tgacgttgat atagacgctt tagaaatgga attggaatca | 840 |
| tccatttggg aaactgtcaa ggaacgtgaa atagaatgta aagatactca caaaaaggat | 900 |
| ctgatgcaat tgattttgga aggggcaatg cgttcatgtg acggtaacct ttgggataaa | 960 |
| tcagcatata gaagatttgt tgtagataat tgtaaatcta tctacttcgc agggcatgat | 1020 |
| agtacagctg tctcagtgtc atggtgtttg atgttactgg ccctaaaccc atcatggcaa | 1080 |
| gttaagatcc gtgatgaaat tctgtcttct tgcaaaaatg gtattccaga tgccgaaagt | 1140 |
| atcccaaacc ttaaaacagt gactatggtt attcaagaga caatgagatt ataccctcca | 1200 |
| gcaccaatcg tcgggagaga agcctctaaa gatatcagat tgggcgatct agttgttcct | 1260 |
| aaaggcgtct gtatatggac actaatacca gctttacaca gagatcctga gatttgggga | 1320 |
| ccagatgcaa acgatttcaa accagaaaga ttttctgaag gaatttcaaa ggcttgtaag | 1380 |
| tatcctcaaa gttacattcc atttggtctg ggtcctagaa catgcgttgg taaaaacttt | 1440 |
| ggcatgatgg aagtaaaggt tcttgtttcc ctgattgtct ccaagttctc tttcactcta | 1500 |
| tctcctacct accaacatag tcctagtcac aaacttttag tagaaccaca acatggggtg | 1560 |
| gtaattagag tggtttaa | 1578 |

SEQ ID NO: 110
A. thaliana

| | |
|---|---|
| MESLVVHTVN AIWCIVIVGI FSVGYHVYGR AVVEQWRMRR SLKLQGVKGP PPSIFNGNVS | 60 |
| EMQRIQSEAK HCSGDNIISH DYSSSLFPHF DHWRKQYGRI YTYSTGLKQH LYINHPEMVK | 120 |
| ELSQTNTLNL GRITHITKRL NPILGNGIIT SNGPHWAHQR RIIAYEFTHD KIKGMVGLMV | 180 |
| ESAMPMLNKW EEMVKRGGEM GCDIRVDEDL KDVSADVIAK ACFGSSFSKG KAIFSMIRDL | 240 |
| LTAITKRSVL FRFNGFTDMV FGSKKHGDVD IDALEMELES SIWETVKERE IECKDTHKKD | 300 |
| LMQLILEGAM RSCDGNLWDK SAYRRFVVDN CKSIYFAGHD STAVSVSWCL MLLALNPSWQ | 360 |
| VKIRDEILSS CKNGIPDAES IPNLKTVTMV IQETMRLYPP APIVGREASK DIRLGDLVVP | 420 |
| KGVCIWTLIP ALHRDPEIWG PDANDFKPER FSEGISKACK YPQSYIPFGL GPRTCVGKNF | 480 |
| GMMEVKVLVS LIVSKFSFTL SPTYQHSPSH KLLVEPQHGV VIRVV | 525 |

SEQ ID NO: 111
Artificial Sequence

| | |
|---|---|
| atgtacttcc tactacaata cctcaacatc acaaccgttg gtgtctttgc cacattgttt | 60 |
| ctctcttatt gtttacttct ctggagaagt agagcgggta acaaaaagat tgccccagaa | 120 |
| gctgccgctg catggcctat tatcggccac ctccacttac ttgcaggtgg atcccatcaa | 180 |
| ctaccacata ttacattggg taacatggca gataagtacg gtcctgtatt cacaatcaga | 240 |
| ataggcttgc atagagctgt agttgtctca tcttgggaaa tggcaaagga atgttcaaca | 300 |
| gctaatgatc aagtgtcttc ttcaagacct gaactattag cttctaagtt gttgggttat | 360 |
| aactacgcca tgtttggttt ttcaccatac ggttcatact ggagagaaat gagaaagatc | 420 |
| atctctctcg aattactatc taattccaga ttggaactat tgaaagatgt tagagcctca | 480 |
| gaagttgtca catctattaa ggaactatac aaattgtggg cggaaaagaa gaatgagtca | 540 |
| ggattggttt ctgtcgagat gaaacaatgg ttcggagatt tgactttaaa cgtgatcttg | 600 |
| agaatggtgg ctggtaaaag atacttctcc gcgagtgacg cttcagaaaa caaacaggcc | 660 |
| cagcgttgta gaagagtctt cagagaattc ttccatctct ccggcttgtt tgtggttgct | 720 |
| gatgctatac cttttcttgg atggctcgat tggggaagac acgagaagac cttgaaaaag | 780 |
| accgccatag aaatggattc catcgcccag gagtggcttg aggaacatag acgtagaaaa | 840 |
| gattctggag atgataattc tacccaagat ttcatggacg ttatgcaatc tgtgctagat | 900 |
| ggcaaaaatc taggcggata cgatgctgat acgattaaca aggctacatg cttaactctt | 960 |
| atatcaggtg gcagtgatac tactgtagtt tctttgacat gggctcttag tcttgtgtta | 1020 |
| aacaatagag atactttgaa aaaggcacag gaagagttag acatccaagt cggtaaggaa | 1080 |
| agattggtta acgagcaaga catcagtaag ttagtttact tgcaagcaat agtaaaagag | 1140 |
| acactcagac tttatccacc aggtcctttg ggtggtttga gacaattcac tgaagattgt | 1200 |
| acactaggtg gctatcacgt ttcaaaagga actagattaa tcatgaactt atccaagatt | 1260 |
| caaaaagatc cacgtatttg gtctgatcct actgaattcc aaccagagag attccttacg | 1320 |
| actcataaag atgtcgatcc acgtggtaaa cactttgaat tcattccatt cggtgcagga | 1380 |
| agacgtgcat gtcctggtat cacattcgga ttacaagtac tacatctaac attggcatct | 1440 |
| ttcttgcatg cgtttgaatt ttcaacacca tcaaatgagc aggttaacat gagagaatca | 1500 |
| ttaggtctta cgaatatgaa atctacccca ttagaagttt tgatttctcc aagactatcc | 1560 |
| cttaattgct tcaaccttat gaaaatttga | 1590 |

SEQ ID NO: 112
V. vinifera

| | |
|---|---|
| MYFLLQYLNI TTVGVFATLF LSYCLLLWRS RAGNKKIAPE AAAAWPIIGH LHLLAGGSHQ | 60 |
| LPHITLGNMA DKYGPVFTIR IGLHRAVVVS SWEMAKECST ANDQVSSSRP ELLASKLLGY | 120 |
| NYAMFGFSPY GSYWREMRKI ISLELLSNSR LELLKDVRAS EVVTSIKELY KLWAEKKNES | 180 |
| GLVSVEMKQW FGDLTLNVIL RMVAGKRYFS ASDASENKQA QRCRRVFREF FHLSGLFVVA | 240 |
| DAIPFLGWLD WGRHEKTLKK TAIEMDSIAQ EWLEEHRRRK DSGDDNSTQD FMDVMQSVLD | 300 |
| GKNLGGYDAD TINKATCLTL ISGGSDTTVV SLTWALSLVL NNRDTLKKAQ EELDIQVGKE | 360 |
| RLVNEQDISK LVYLQAIVKE TLRLYPPGPL GGLRQFTEDC TLGGYHVSKG TRLIMNLSKI | 420 |
| QKDPRIWSDP TEFQPERFLT THKDVDPRGK HFEFIPFGAG RRACPGITFG LQVLHLTLAS | 480 |
| FLHAFEFSTP SNEQVNMRES LGLTNMKSTP LEVLISPRLS SCSLYN | 526 |

SEQ ID NO: 113
Artificial Sequence

| | |
|---|---|
| atggaaccta acttttactt gtcattacta ttgttgttcg tgaccttcat ttctttaagt | 60 |
| ctgtttttca tcttttacaa acaaaagtcc ccattgaatt tgccaccagg gaaaatgggt | 120 |
| taccctatca taggtgaaag tttagaattc ctatccacag gctggaaggg acatcctgaa | 180 |
| aagttcatat ttgatagaat gcgtaagtac agtagtgagt tattcaagac ttctattgta | 240 |
| ggcgaatcca cagttgtttg ctgtggggca gctagtaaca aattcctatt ctctaacgaa | 300 |
| aacaaactgg taactgcctg gtggccagat tctgttaaca aaatcttccc aacaacttca | 360 |
| ctggattcta atttgaagga ggaatctata aagatgagaa agttgctgcc acagttcttc | 420 |
| aaaccagaag cacttcaaag atacgtcggc gttatggatg taatcgcaca aagacatttt | 480 |
| gtcactcact gggacaacaa aaatgagatc acagtttatc cacttgctaa aagatacact | 540 |
| ttcttgcttg cgtgtagact gttcatgtct gttgaggatg aaaatcatgt ggcgaaattc | 600 |
| tcagacccat tccaactaat cgctgcaggc atcatttcac ttcctatcga tcttcctggt | 660 |
| actccattca acaaggccat aaaggcttca aatttcatta gaaaagagct gataaagatt | 720 |
| atcaaacaaa gacgtgttga tctggcagag ggtacagcat ctccaaccca ggatatcttg | 780 |
| tcacatatgc tattaacatc tgatgaaaac ggtaaatcta tgaacgagtt gaacattgcc | 840 |
| gacaagattc ttggactatt gataggaggc cacgatacag cttcagtagc ttgcacattt | 900 |
| ctagtgaagt acttaggaga attaccacat atctacgata aagtctacca agagcaaatg | 960 |
| gaaattgcca agtccaaacc tgctggggaa ttgttgaatt gggatgactt gaaaaagatg | 1020 |
| aagtattcat ggaatgtggc atgtgaggta atgagattgt caccaccttt acaaggtggt | 1080 |
| tttagagagg ctataactga ctttatgttt aacggtttct ctattccaaa agggtggaag | 1140 |
| ttatactggt ccgccaactc tacacacaaa aatgcagaat gtttcccaat gcctgagaaa | 1200 |
| ttcgatccta ccagatttga aggtaatggt ccagcgcctt atacatttgt accattcggt | 1260 |
| ggaggcccta gaatgtgtcc tggaaaggaa tacgctagat tagaaatctt ggttttcatg | 1320 |
| cataatctgg tcaaacgttt taagtgggaa aaggttattc cagacgaaaa gattattgtc | 1380 |
| gatccattcc caatcccagc taaagatctt ccaatccgtt tgtatcctca caaagcttaa | 1440 |

SEQ ID NO: 114
M. truncatula

| | |
|---|---|
| MEPNFYLSLL LLFVTFISLS LFFIFYKQKS PLNLPPGKMG YPIIGESLEF LSTGWKGHPE | 60 |
| KFIFDRMRKY SSELFKTSIV GESTVVCCGA ASNKFLFSNE NKLVTAWWPD SVNKIFPTTS | 120 |
| LDSNLKEESI KMRKLLPQFF KPEALQRYVG VMDVIAQRHF VTHWDNKNEI TVYPLAKRYT | 180 |
| FLLACRLFMS VEDENHVAKF SDPFQLIAAG IISLPIDLPG TPFNKAIKAS NFIRKELIKI | 240 |
| IKQRRVDLAE GTASPTQDIL SHMLLTSDEN GKSMNELNIA DKILGLLIGG HDTASVACTF | 300 |
| LVKYLGELPH IYDKVYQEQM EIAKSKPAGE LLNWDDLKKM KYSWNVACEV MRLSPPLQGG | 360 |
| FREAITDFMF NGFSIPKGWK LYWSANSTHK NAECFPMPEK FDPTRFEGNG PAPYTFVPFG | 420 |
| GGPRMCPGKE YARLEILVFM HNLVKRFKWE KVIPDEKIIV DPFPIPAKDL PIRLYPHKA | 479 |

SEQ ID NO: 115
Artificial Sequence

| | |
|---|---|
| atggcctctg ttactttggg ttcctggatc gtcgtccacc accataacca tcaccatcca | 60 |
| tcatctatcc taactaaatc tcgttcaaga tcctgtccta ttacactaac caaaccaatc | 120 |
| tcttttcgtt caaagagaac agtttcctct agtagttcta tcgtgtcctc tagtgtcgtc | 180 |
| actaaggaag acaatctgag acagtctgaa ccttcttcct ttgatttcat gtcatatatc | 240 |
| attactaagg cagaactagt gaataaggct cttgattcag cagttccatt aagagagcca | 300 |
| ttgaaaatcc atgaagcaat gagatactct cttctagctg gcgggaagag agtcagacct | 360 |
| gtactctgca tagcagcgtg cgaattagtt ggtggcgagg aatcaaccgc tatgcctgcc | 420 |
| gcttgtgctg tagaaatgat tcatacaatg tcactgatac acgatgattt gccatgtatg | 480 |
| gataacgatg atctgagaag gggtaagcca actaaccata aggttttcgg cgaagatgtt | 540 |
| gccgtcttag ctggtgatgc tttgttatct ttcgcgttcg aacatttggc atccgcaaca | 600 |
| tcaagtgatg ttgtgtcacc agtaagagta gttagagcag ttggagaact ggctaaagct | 660 |
| attggaactg agggtttagt tgcaggtcaa gtcgtcgata tctcttccga aggtcttgat | 720 |
| ttgaatgatg taggtcttga acatctcgaa ttcatccatc ttcacaagac agctgcactt | 780 |
| ttagaagcca gtgcggttct cggcgcaatt gttggcggag ggagtgatga cgaaattgag | 840 |
| agattgagga agtttgctag atgtatagga ttactgttcc aagtagtaga cgatatacta | 900 |
| gatgtgacaa agtcttccaa agagttggga aaaacagctg gtaaagattt gattgccgac | 960 |
| aaattgacct accctaagat tatggggcta gaaaaatcaa gagaatttgc cgagaaactc | 1020 |
| aatagagagg cgcgtgatca actgttgggt ttcgattctg ataaagttgc accactctta | 1080 |
| gccttagcca actacatcgc ttacagacaa aactaa | 1116 |

SEQ ID NO: 116
A. thaliana

| | |
|---|---|
| MASVTLGSWI VVHHHNHHHP SSILTKSRSR SCPITLTKPI SFRSKRTVSS SSSIVSSSVV | 60 |
| TKEDNLRQSE PSSFDFMSYI ITKAELVNKA LDSAVPLREP LKIHEAMRYS LLAGGKRVRP | 120 |
| VLCIAACELV GGEESTAMPA ACAVEMIHTM SLIHDDLPCM DNDDLRRGKP TNHKVFGEDV | 180 |
| AVLAGDALLS FAFEHLASAT SSDVVSPVRV VRAVGELAKA IGTEGLVAGQ VVDISSEGLD | 240 |
| LNDVGLEHLE FIHLHKTAAL LEASAVLGAI VGGGSDDEIE RLRKFARCIG LLFQVVDDIL | 300 |
| DVTKSSKELG KTAGKDLIAD KLTYPKIMGL EKSREFAEKL NREARDQLLG FDSDKVAPLL | 360 |
| ALANYIAYRQ N | 371 |

SEQ ID NO: 117
R. suavissimus

| | |
|---|---|
| MATLLEHFQA MPFAIPIALA ALSWLFLFYI KVSFFSNKSA QAKLPPVPVV PGLPVIGNLL | 60 |
| QLKEKKPYQT FTRWAEEYGP IYSIRTGAST MVVLNTTQVA KEAMVTRYLS ISTRKLSNAL | 120 |
| KILTADKCMV AISDYNDFHK MIKRYILSNV LGPSAQKRHR SNRDTLRANV CSRLHSQVKN | 180 |
| SPREAVNFRR VFEWELFGIA LKQAFGKDIE KPIYVEELGT TLSRDEIFKV LVLDIMEGAI | 240 |
| EVDWRDFFPY LRWIPNTRME TKIQRLYFRR KAVMTALINE QKKRIASGEE INCYIDFLLK | 300 |
| EGKTLTMDQI SMLLWETVIE TADTTMVTTE WAMYEVAKDS KRQDRLYQEI QKVCGSEMVT | 360 |
| EEYLSQLPYL NAVFHETLRK HSPAALVPLR YAHEDTQLGG YYIPAGTEIA INIYGCNMDK | 420 |
| HQWESPEEWK PERFLDPKFD PMDLYKTMAF GAGKRVCAGS LQAMLIACPT IGRLVQEFEW | 480 |
| KLRDGEEENV DTVGLTTHKR YPMHAILKPR S | 511 |

SEQ ID NO: 118
S. cerevisiae

| | |
|---|---|
| atgtcatttc aaattgaaac ggttcccacc aaaccatatg aagaccaaaa gcctggtacc | 60 |
| tctggtttgc gtaagaagac aaaggtgttt aaagacgaac ctaactacac agaaaatttc | 120 |
| attcaatcga tcatggaagc tattccagag ggttctaaag gtgccactct tgttgtcggt | 180 |
| ggtgatgggc gttactacaa tgatgtcatt cttcataaga ttgccgctat cggtgctgcc | 240 |
| aacggtatta aaaagttagt tattggccag catggtcttc tgtctacgcc agccgcttct | 300 |
| cacatcatga gaacctacga ggaaaaatgt actggtggta ttatcttaac cgcctcacat | 360 |
| aatccaggtg gtccagaaaa tgacatgggt attaagtata acttatccaa tgggggtcct | 420 |
| gctcctgaat ccgtcacaaa tgctatttgg gagatttcca aaaagcttac cagctataag | 480 |
| attatcaaag acttcccaga actagacttg ggtacgatag gcaagaacaa gaaatacggt | 540 |
| ccattactcg ttgacattat cgatattaca aaagattatg tcaacttctt gaaggaaatc | 600 |
| ttcgatttcg acttaatcaa gaaattcatc gataatcaac gttctactaa gaattggaag | 660 |
| ttactgtttg acagtatgaa cggtgtaact ggaccatacg gtaaggctat tttcgttgat | 720 |
| gaatttggtt taccggcgga tgaggtttta caaaactggc atccttctcc ggattttggt | 780 |
| ggtatgcatc cagatccaaa cttaacttat gccagttcgt tagtgaaaag agtagatcgt | 840 |
| gaaaagattg agtttggtgc tgcatccgat ggtgatggtg atagaaatat gatttacggt | 900 |
| tacggcccat ctttcgtttc tccaggtgac tccgtcgcaa ttattgccga atatgcagct | 960 |
| gaaatcccat atttcgccaa gcaaggtata tatggtctgg cccgttcatt ccctacctca | 1020 |
| ggagccatag accgtgttgc caaggcccat ggtctaaact gttatgaggt cccaactggc | 1080 |
| tggaaatttt tctgtgcttt gttcgacgct aaaaaattat ctatttgtgg tgaagaatcg | 1140 |
| tttggtactg gttccaacca cgtaagggaa aaggacggtg tttgggccat tatggcgtgg | 1200 |
| ttgaacatct tggccattta caacaagcat catccggaga acgaagcttc tattaagacg | 1260 |
| atacagaatg aattctgggc aaagtacggc cgtactttct tcactcgtta tgattttgaa | 1320 |
| aaagttgaaa cagaaaaagc taacaagatt gtcgatcaat tgagagcata tgttaccaaa | 1380 |
| tcgggtgttg ttaattccgc cttcccagcc gatgagtctc ttaaggtcac cgattgtggt | 1440 |
| gatttttcat acacagattt ggacggttct gtttctgacc atcaaggttt atatgtcaag | 1500 |
| ctttccaatg gtgcaagatt cgttctaaga ttgtcaggta caggttcttc aggtgctacc | 1560 |
| attagattgt acattgaaaa atactgcgat gataaatcac aataccaaaa gacagctgaa | 1620 |
| gaatacttga agccaattat taactcggtc atcaagttct tgaactttaa acaagtttta | 1680 |
| ggaactgaag aaccaacggt tcgtacttaa | 1710 |

SEQ ID NO: 119
S. cerevisiae

| | |
|---|---|
| MSFQIETVPT KPYEDQKPGT SGLRKKTKVF KDEPNYTENF IQSIMEAIPE GSKGATLVVG | 60 |
| GDGRYYNDVI LHKIAAIGAA NGIKKLVIGQ HGLLSTPAAS HIMRTYEEKC TGGIILTASH | 120 |
| NPGGPENDMG IKYNLSNGGP APESVTNAIW EISKKLTSYK IIKDFPELDL GTIGKNKKYG | 180 |
| PLLVDIIDIT KDYVNFLKEI FDFDLIKKFI DNQRSTKNWK LLFDSMNGVT GPYGKAIFVD | 240 |
| EFGLPADEVL QNWHPSPDFG GMHPDPNLTY ASSLVKRVDR EKIEFGAASD GDGDRNMIYG | 300 |
| YGPSFVSPGD SVAIIAEYAA EIPYFAKQGI YGLARSFPTS GAIDRVAKAH GLNCYEVPTG | 360 |
| WKFFCALFDA KKLSICGEES FGTGSNHVRE KDGVWAIMAW LNILAIYNKH HPENEASIKT | 420 |
| IQNEFWAKYG RTFFTRYDFE KVETEKANKI VDQLRAYVTK SGVVNSAFPA DESLKVTDCG | 480 |
| DFSYTDLDGS VSDHQGLYVK LSNGARFVLR LSGTGSSGAT IRLYIEKYCD DKSQYQKTAE | 540 |
| EYLKPIINSV IKFLNFKQVL GTEEPTVRT | 569 |

SEQ ID NO: 120
S. cerevisiae

| | |
|---|---|
| atgtccacta agaagcacac caaaacacat tccacttatg cattcgagag caacacaaac | 60 |
| agcgttgctg cctcacaaat gagaaacgcc ttaaacaagt tggcggactc tagtaaactt | 120 |
| gacgatgctg ctcgcgctaa gtttgagaac gaactggatt cgtttttcac gcttttcagg | 180 |
| agatatttgg tagagaagtc ttctagaacc accttggaat gggacaagat caagtctccc | 240 |
| aacccggatg aagtggttaa gtatgaaatt atttctcagc agcccgagaa tgtctcaaac | 300 |
| ctttccaaat tggctgtttt gaagttgaac ggtgggctgg gtacctccat gggctgcgtt | 360 |
| ggccctaaat ctgttattga agtgagagag ggaaacacct ttttggattt gtctgttcgt | 420 |
| caaattgaat acttgaacag acagtacgat agcgacgtgc cattgttatt gatgaattct | 480 |
| ttcaacactg acaaggatac ggaacacttg attaagaagt attccgctaa cagaatcaga | 540 |
| atcagatctt tcaatcaatc caggttccca agagtctaca aggattcttt attgcctgtc | 600 |
| cccaccgaat acgattctcc actggatgct tggtatccac caggtcacgg tgatttgttt | 660 |
| gaatctttac acgtatctgg tgaactggat gccttaattg cccaaggaag agaaatatta | 720 |
| tttgtttcta acggtgacaa cttgggtgct accgtcgact taaaaatttt aaaccacatg | 780 |
| atcgagactg gtgccgaata tataatggaa ttgactgata agaccagagc cgatgttaaa | 840 |
| ggtggtactt tgatttctta cgatggtcaa gtccgtttat tggaagtcgc ccaagttcca | 900 |
| aaagaacaca ttgacgaatt caaaaatatc agaaagttta ccaacttcaa cacgaataac | 960 |
| ttatggatca atctgaaagc agtaaagagg ttgatcgaat cgagcaattt ggagatggaa | 1020 |
| atcattccaa accaaaaaac tataacaaga gacggtcatg aaattaatgt cttacaatta | 1080 |
| gaaaccgctt gtggtgctgc tatcaggcat tttgatggtg ctcacggtgt tgtcgttcca | 1140 |
| agatcaagat tcttgcctgt caagacctgt tccgatttgt tgctggttaa atcagatcta | 1200 |
| ttccgtctgg aacacggttc tttgaagtta gacccatccc gttttggtcc aaacccatta | 1260 |
| atcaagttgg gctcgcattt caaaaaggtt tctggtttta acgcaagaat ccctcacatc | 1320 |
| ccaaaaatcg tcgagctaga tcatttgacc atcactggta acgtcttttt aggtaaagat | 1380 |
| gtcactttga ggggtactgt catcatcgtt tgctccgacg gtcataaaat cgatattcca | 1440 |
| aacggctcca tattggaaaa tgttgtcgtt actggtaatt tgcaaatctt ggaacattga | 1500 |

SEQ ID NO: 121
S. cerevisiae

| | |
|---|---|
| MSTKKHTKTH STYAFESNTN SVAASQMRNA LNKLADSSKL DDAARAKFEN ELDSFFTLFR | 60 |
| RYLVEKSSRT TLEWDKIKSP NPDEVVKYEI ISQQPENVSN LSKLAVLKLN GGLGTSMGCV | 120 |
| GPKSVIEVRE GNTFLDLSVR QIEYLNRQYD SDVPLLLMNS FNTDKDTEHL IKKYSANRIR | 180 |
| IRSFNQSRFP RVYKDSLLPV PTEYDSPLDA WYPPGHGDLF ESLHVSGELD ALIAQGREIL | 240 |
| FVSNGDNLGA TVDLKILNHM IETGAEYIME LTDKTRADVK GGTLISYDGQ VRLLEVAQVP | 300 |
| KEHIDEFKNI RKFTNFNTNN LWINLKAVKR LIESSNLEME IIPNQKTITR DGHEINVLQL | 360 |
| ETACGAAIRH FDGAHGVVVP RSRFLPVKTC SDLLLVKSDL FRLEHGSLKL DPSRFGPNPL | 420 |
| IKLGSHFKKV SGFNARIPHI PKIVELDHLT ITGNVFLGKD VTLRGTVIIV CSDGHKIDIP | 480 |
| NGSILENVVV TGNLQILEH | 499 |

SEQ ID NO: 122
S. cerevisiae

| | |
|---|---|
| atgtctagtc aaacagaaag aacttttatt gcggtaaaac cagatggtgt ccagaggggc | 60 |
| ttagtatctc aaattctatc tcgttttgaa aaaaaaggtt acaaactagt tgctattaaa | 120 |
| ttagttaaag cggatgataa attactagag caacattacg cagagcatgt tggtaaacca | 180 |
| tttttcccaa agatggtatc ctttatgaag tctggtccca ttttggccac ggtctgggag | 240 |
| ggaaaagatg tggttagaca aggaagaact attcttggtg ctactaatcc tttgggcagt | 300 |
| gcaccaggta ccattagagg tgatttcggt attgacctag gcagaaacgt ctgtcacggc | 360 |
| agtgattctg ttgatagcgc tgaacgtgaa atcaatttgt ggtttaagaa ggaagagtta | 420 |
| gttgattggg aatctaatca agctaagtgg atttatgaat ga | 462 |

SEQ ID NO: 123
S. cerevisiae

| | |
|---|---|
| MSSQTERTFI AVKPDGVQRG LVSQILSRFE KKGYKLVAIK LVKADDKLLE QHYAEHVGKP | 60 |
| FFPKMVSFMK SGPILATVWE GKDVVRQGRT ILGATNPLGS APGTIRGDFG IDLGRNVCHG | 120 |
| SDSVDSAERE INLWFKKEEL VDWESNQAKW IYE | 153 |

SEQ ID NO: 124
S. rebaudiana

| | |
|---|---|
| atggctgctg ctgatactga aaagttgaac aatttgagat ccgccgtttc tggtttgacc | 60 |
| caaatttctg ataacgaaaa gtccggtttc atcaacttgg tcagtagata tttgtctggt | 120 |
| gaagctcaac acgttgaatg gtctaaaatt caaactccaa ccgataagat cgttgttcca | 180 |
| tacgatactt tgtctgctgt tccagaagat gctgctcaaa caaaatcttt gttggataag | 240 |
| ttggtcgtct tgaagttgaa cggtggtttg ggtactacta tgggttgtac tggtccaaag | 300 |
| tctgttatcg aagttagaaa cggtttgacc ttcttggatt tgatcgtcat ccaaatcgaa | 360 |
| tccttgaaca agaagtacgg ttgttctgtt cctttgttgt tgatgaactc tttcaacacc | 420 |
| catgaagata cccaaaagat cgtcgaaaag tactccggtt ctaacattga agttcacacc | 480 |
| ttcaatcaat cccaataccc aagattggtt gtcgatgaat ttttgccatt gccatctaaa | 540 |
| ggtgaaactg gtaaagatgg ttggtatcca ccaggtcatg gtgatgtttt tccatccttg | 600 |
| atgaattccg gtaagttgga tgctttgttg tcccaaggta aagaatacgt tttcgttgcc | 660 |
| aactctgata acttgggtgc agttgttgat ttgaagatct tgaaccactt gatccaaaac | 720 |
| aagaacgaat actgcatgga agttactcca aagactttgg ctgatgttaa gggtggtact | 780 |
| ttgatttctt acgatggtaa ggttcaatta ttggaaatcg cccaagttcc agatgaacac | 840 |
| gttaatgaat tcaagtccat cgaaaagttt aagatcttta acactaacaa cttgtgggtc | 900 |
| aacttgaacg ccattaagag attggttcaa gctgatgctt tgaagatgga aattattcca | 960 |
| aatccaaaag aagtcaacgg tgtcaaggta ttgcaattgg aaactgctgc tggtgctgct | 1020 |
| attaagtttt tcgataatgc catcggtatc aacgtcccaa gatctagatt tttgcctgtt | 1080 |
| aaggcttcct ctgacttgtt gttagttcaa tcagacttgt acaccgaaaa ggatggttac | 1140 |
| gttattagaa acccagctag aaaggatcca gctaacccat ctattgaatt gggtccagaa | 1200 |
| ttcaaaaagg tcggtgattt cttgaagaga ttcaagtcta tcccatccat catcgaattg | 1260 |
| gactcattga aagtttctgg tgatgtctgg tttggttcca acgttgtttt gaaaggtaag | 1320 |
| gttgttgttg ctgccaaatc cggtgaaaaa ttggaaattc cagatggtgc cttgattgaa | 1380 |
| aacaaagaag ttcatggtgc ctccgacatt tga | 1413 |

SEQ ID NO: 125
S. rebaudiana

| | |
|---|---|
| MAAADTEKLN NLRSAVSGLT QISDNEKSGF INLVSRYLSG EAQHVEWSKI QTPTDKIVVP | 60 |
| YDTLSAVPED AAQTKSLLDK LVVLKLNGGL GTTMGCTGPK SVIEVRNGLT FLDLIVIQIE | 120 |
| SLNKKYGCSV PLLLMNSFNT HEDTQKIVEK YSGSNIEVHT FNQSQYPRLV VDEFLPLPSK | 180 |
| GETGKDGWYP PGHGDVFPSL MNSGKLDALL SQGKEYVFVA NSDNLGAVVD LKILNHLIQN | 240 |
| KNEYCMEVTP KTLADVKGGT LISYDGKVQL LEIAQVPDEH VNEFKSIEKF KIFNTNNLWV | 300 |
| NLNAIKRLVQ ADALKMEIIP NPKEVNGVKV LQLETAAGAA IKFFDNAIGI NVPRSRFLPV | 360 |
| KASSDLLLVQ SDLYTEKDGY VIRNPARKDP ANPSIELGPE FKKVGDFLKR FKSIPSIIEL | 420 |
| DSLKVSGDVW FGSNVVLKGK VVVAAKSGEK LEIPDGALIE NKEVHGASDI | 470 |

SEQ ID NO: 126
A. pullulans

| | |
|---|---|
| atgtcctctg aaatggctac tcatttgaaa cctaatggtg gtgccgaatt cgaaaaaaga | 60 |
| catcatggta agacccaatc ccatgttgct tttgaaaaca cttctacatc tgttgctgcc | 120 |
| tcccaaatga gaaatgcttt gaatactttg tgcgattccg ttactgatcc agctgaaaag | 180 |
| caaagattcg aaaccgaaat ggataacttc ttcgccttgt ttagaagata cttgaacgat | 240 |
| aaggctaagg gtaacgaaat cgaatggtct agaattgctc caccaaaacc agaacaagtt | 300 |
| gttgcttatc aagacttgcc tgaacaagaa tccgttgaat tcttgaacaa attggccgtc | 360 |
| ttgaagttga atggtggttt gggtacttct atgggttgtg ttggtccaaa gtctgttatc | 420 |
| gaagttagag atggtatgtc cttcttggat ttgtccgtta gacaaatcga atacttgaat | 480 |
| agaacctacg gtgttaacgt tccattcgtc ttgatgaatt ctttcaacac tgatgctgat | 540 |
| accgccaaca ttatcaaaaa gtacgaaggt cacaacatcg acatcatgac cttcaatcaa | 600 |
| tctagatacc caagaatctt gaaggattct ttgttgccag ctccaaaatc tgccaactct | 660 |
| caaatttctg attggtatcc accaggtcat ggtgacgttt ttgaatcctt gtacaactct | 720 |
| ggtatcttgg ataagttgtt ggaaagaggt gtcgaaatcg ttttcttgtc caatgctgat | 780 |
| aatttgggtg ccgttgttga tttgaagatc ttgcaacata tggttgatac caaggccgaa | 840 |
| tatatcatgg aattgactga taagactaag gccgatgtta agggtggtac tattattgac | 900 |
| tatgaaggtc aagccagatt attggaaatt gcccaagttc caaaagaaca cgtcaacgaa | 960 |
| ttcaagtcca tcaagaagtt taagtacttc aacaccaaca acatctggat gaacttgaga | 1020 |
| gctgttaaga gaatcgtcga aaacaacgaa ttggccatgg aaattatccc aaacggtaaa | 1080 |
| tctattccag ccgacaaaaa aggtgaagcc gatgtttcta tagttcaatt ggaaactgct | 1140 |
| gttggtgctg ccattagaca ttttaacaat gctcatggtg tcaacgtccc aagaagaaga | 1200 |
| tttttgccag ttaagacctg ctccgatttg atgttggtta agtctgactt gtacactttg | 1260 |
| aagcacggtc aattgattat ggacccaaat agatttggtc cagccccatt gattaagttg | 1320 |
| ggtggtgatt ttaagaaggt ttcctcattc caatccagaa tcccatccat tcctaaaatc | 1380 |
| ttggaattgg atcatttgac cattaccggt ccagttaact tgggtagagg tgttactttt | 1440 |
| aagggtactg ttattatcgt tgcctccgaa ggtcaaacca ttgatattcc acctggttcc | 1500 |
| attttggaaa acgttgttgt tcaaggttcc ttgagattat tagaacatta a | 1551 |

SEQ ID NO: 127
A. pullulans

| | |
|---|---|
| MSSEMATHLK PNGGAEFEKR HHGKTQSHVA FENTSTSVAA SQMRNALNTL CDSVTDPAEK | 60 |
| QRFETEMDNF FALFRRYLND KAKGNEIEWS RIAPPKPEQV VAYQDLPEQE SVEFLNKLAV | 120 |
| LKLNGGLGTS MGCVGPKSVI EVRDGMSFLD LSVRQIEYLN RTYGVNVPFV LMNSFNTDAD | 180 |
| TANIIKKYEG HNIDIMTFNQ SRYPRILKDS LLPAPKSANS QISDWYPPGH GDVFESLYNS | 240 |
| GILDKLLERG VEIVFLSNAD NLGAVVDLKI LQHMVDTKAE YIMELTDKTK ADVKGGTIID | 300 |
| YEGQARLLEI AQVPKEHVNE FKSIKKFKYF NTNNIWMNLR AVKRIVENNE LAMEIIPNGK | 360 |
| SIPADKKGEA DVSIVQLETA VGAAIRHFNN AHGVNVPRRR FLPVKTCSDL MLVKSDLYTL | 420 |
| KHGQLIMDPN RFGPAPLIKL GGDFKKVSSF QSRIPSIPKI LELDHLTITG PVNLGRGVTF | 480 |
| KGTVIIVASE GQTIDIPPGS ILENVVVQGS LRLLEH | 516 |

SEQ ID NO: 128
A. thaliana

| | |
|---|---|
| atggctgcta ctactgaaaa cttgccacaa ttgaaatctg ccgttgatgg tttgactgaa | 60 |
| atgtccgaat ctgaaaagtc cggtttcatc tctttggtca gtagatattt gtctggtgaa | 120 |
| gcccaacata tcgaatggtc taaaattcaa actccaaccg acgaaatcgt tgtcccatac | 180 |
| gaaaaaatga ctccagtttc tcaagatgtc gccgaaacta agaatttgtt ggataagttg | 240 |
| gtcgtcttga agttgaatgg tggtttgggt actactatgg gttgtactgg tccaaagtct | 300 |
| gttatcgaag ttagagatgg tttaaccttc ttggacttga tcgtcatcca aatcgaaaac | 360 |
| ttgaacaaca agtacggttg caaggttcca ttggtcttga tgaattcttt caacacccat | 420 |
| gatgataccc acaagatcgt tgaaaagtac accaactcca acgttgatat ccacaccttc | 480 |
| aatcaatcta agtacccaag agttgttgcc gatgaatttg ttccatggcc atctaaaggt | 540 |
| aagactgaca aagaaggttg gtatccacca ggtcatggtg atgtttttcc agctttaatg | 600 |
| aactccggta agttggatac tttcttgtcc caaggtaaag aatacgtttt cgttgccaac | 660 |
| tctgataact tgggtgctat agttgatttg accatcttga agcacttgat ccaaaacaag | 720 |
| aacgaatact gcatggaagt tactccaaag actttggctg atgttaaggg tggtactttg | 780 |
| atttcttacg aaggtaaggt tcaattattg gaaatcgccc aagttccaga tgaacacgtt | 840 |
| aatgaattca agtccatcga aaagttcaag atcttcaaca ccaacaactt gtgggttaac | 900 |
| ttgaaggcca tcaagaaatt ggttgaagct gatgctttga agatggaaat tatcccaaac | 960 |
| ccaaaagaag ttgacggtgt taaggtattg caattggaaa ctgctgctgg tgctgctatt | 1020 |
| agatttttcg ataatgccat cggtgttaac gtcccaagat ctagattttt gccagttaag | 1080 |
| gcttcctccg atttgttgtt ggttcaatct gacttgtaca ccttggttga cggttttgtt | 1140 |
| acaagaaaca aggctagaac taacccatcc aacccatcta ttgaattggg tccagaattc | 1200 |
| aaaaaggttg ccacattctt gtccagattc aagtctattc catccatcgt cgaattggac | 1260 |
| tcattgaaag tttctggtga tgtctggttt ggttcctcta tagttttgaa gggtaaggtt | 1320 |
| actgttgctg ctaaatctgg tgttaagttg gaaattccag atagagccgt tgtcgaaaac | 1380 |
| aaaaacatta acggtcctga agatttgtga | 1410 |

SEQ ID NO: 129
A. thaliana

| | |
|---|---|
| MAATTENLPQ LKSAVDGLTE MSESEKSGFI SLVSRYLSGE AQHIEWSKIQ TPTDEIVVPY | 60 |
| EKMTPVSQDV AETKNLLDKL VVLKLNGGLG TTMGCTGPKS VIEVRDGLTF LDLIVIQIEN | 120 |
| LNNKYGCKVP LVLMNSFNTH DDTHKIVEKY TNSNVDIHTF NQSKYPRVVA DEFVPWPSKG | 180 |
| KTDKEGWYPP GHGDVFPALM NSGKLDTFLS QGKEYVFVAN SDNLGAIVDL TILKHLIQNK | 240 |
| NEYCMEVTPK TLADVKGGTL ISYEGKVQLL EIAQVPDEHV NEFKSIEKFK IFNTNNLWVN | 300 |
| LKAIKKLVEA DALKMEIIPN PKEVDGVKVL QLETAAGAAI RFFDNAIGVN VPRSRFLPVK | 360 |
| ASSDLLLVQS DLYTLVDGFV TRNKARTNPS NPSIELGPEF KKVATFLSRF KSIPSIVELD | 420 |
| SLKVSGDVWF GSSIVLKGKV TVAAKSGVKL EIPDRAVVEN KNINGPEDL | 469 |

SEQ ID NO: 130
E. coli

| | |
|---|---|
| atggctgcta ttaacaccaa ggttaagaag gctgttattc cagttgctgg tttgggtact | 60 |
| agaatgttgc cagctacaaa agccattcca aaagaaatgt taccattggt cgataagcca | 120 |
| ttgatccaat acgttgtcaa cgaatgtatt gctgctggta ttaccgaaat cgttttggtt | 180 |
| actcactcct ccaagaactc cattgaaaat catttcgaca cctcattcga attggaagcc | 240 |
| atgttggaaa agagagtcaa gagacaatta ttggacgaag tccaatctat ttgcccacca | 300 |
| catgttacta tcatgcaagt tagacaaggt ttggctaaag gtttgggtca tgctgttttg | 360 |
| tgtgctcatc cagttgttgg tgatgaacca gttgcagtta ttttgccaga tgttatcttg | 420 |
| gacgaatacg aatccgattt gtctcaagat aacttggctg aaatgatcag aagattcgac | 480 |
| gaaactggtc actcccaaat tatggttgaa cctgttgctg atgttactgc ttatggtgtt | 540 |
| gttgattgca agggtgttga attggctcca ggtgaatctg ttccaatggt tggtgttgta | 600 |
| gaaaagccaa aagctgatgt tgctccatct aatttggcta tcgttggtag atatgttttg | 660 |
| tccgctgata tttggccttt gttggctaaa actccaccag gtgctggtga cgaaattcaa | 720 |
| ttgactgatg ctatcgacat gttgatcgaa aaagaaaccg ttgaagccta ccacatgaag | 780 |
| ggtaaatctc atgattgtgg taacaagttg ggttacatgc aagcttttgt tgaatacggt | 840 |
| atcagacata acaccttagg tactgaattc aaggcttggt tggaagaaga aatgggtatc | 900 |
| aagaagtaa | 909 |

SEQ ID NO: 131
E. coli

| | |
|---|---|
| MAAINTKVKK AVIPVAGLGT RMLPATKAIP KEMLPLVDKP LIQYVVNECI AAGITEIVLV | 60 |
| THSSKNSIEN HFDTSFELEA MLEKRVKRQL LDEVQSICPP HVTIMQVRQG LAKGLGHAVL | 120 |
| CAHPVVGDEP VAVILPDVIL DEYESDLSQD NLAEMIRRFD ETGHSQIMVE PVADVTAYGV | 180 |
| VDCKGVELAP GESVPMVGVV EKPKADVAPS NLAIVGRYVL SADIWPLLAK TPPGAGDEIQ | 240 |
| LTDAIDMLIE KETVEAYHMK GKSHDCGNKL GYMQAFVEYG IRHNTLGTEF KAWLEEEMGI | 300 |
| KK | 302 |

SEQ ID NO: 132
R. suavissimus

| | |
|---|---|
| atggctgctg ttgctactga taagatctct aagttgaagt ctgaagttgc tgccttgtcc | 60 |
| caaatttctg aaaacgaaaa gtccggtttc atcaacttgg tcagtagata tttgtctggt | 120 |
| actgaagcta ctcacgttga atggtctaaa attcaaactc caaccgatga agttgttgtt | 180 |
| ccatatgata ctttggctcc aactccagaa gatccagctg aaactaagaa gttgttagat | 240 |
| aagttggtcg tcttgaagtt gaacggtggt ttgggtacta ctatgggttg tactggtcca | 300 |
| aagtctgtta tcgaagttag aaacggtttg accttcttgg atttgatcgt cattcaaatc | 360 |
| gaaaccttga acaacaagta cggttgtaac gttcctttgt tgttgatgaa ctctttcaac | 420 |
| acccatgatg acaccttcaa gatcgttgaa agatacacca agtccaacgt tcaaatccat | 480 |
| accttcaatc aatcccaata cccaagattg gttgtcgaag ataattctcc attgccatct | 540 |
| aagggtcaaa ctggtaaaga tggttggtat ccaccaggtc atggtgatgt ttttccatct | 600 |
| ttgagaaact ccggtaagtt ggatttgttg ttatcccaag gtaaagaata cgttttcatc | 660 |
| tccaactctg ataacttggg tgcagttgtt gatttgaaga tcttgtccca tttggtccaa | 720 |
| aaaaagaacg aatactgcat ggaagttacc ccaaaaactt tggctgatgt taagggtggt | 780 |
| actttgattt cttacgaagg tagaacccaa ttattggaaa ttgcccaagt tccagatcaa | 840 |
| cacgttaacg aattcaagtc catcgaaaag ttcaagatct ttaacaccaa caatttgtgg | 900 |
| gtcaacttga acgccattaa gagattagtt gaagctgatg ccttgaaaat ggaaatcatc | 960 |
| ccaaatccaa aagaagtcga cggtattaag gtcttgcaat tggaaactgc tgctggtgct | 1020 |
| gctattagat ttttcaatca tgccatcggt atcaacgtcc caagatctag atttttgcca | 1080 |
| gttaaggcta cctccgattt gttattggtt caatctgact tgtacaccgt cgaagatggt | 1140 |
| ttcgttatta gaaacactgc tagaaagaat ccagccaacc catctgttga attgggtcca | 1200 |
| gaattcaaaa aggttgccaa cttcttgtcc agattcaagt ctattccatc catcatcgaa | 1260 |
| ttggactcat tgaaggttgt tggtgatgta tggtttggtg ctggtgttgt tttgaaaggt | 1320 |
| aaggttacta ttactgctaa gccaggtgtt aagttggaaa ttccagataa ggctgtcttg | 1380 |
| gaaaacaagg atattaacgg tcctgaagat ttgtga | 1416 |

SEQ ID NO: 133
R. suavissimus

| | |
|---|---|
| MAAVATDKIS KLKSEVAALS QISENEKSGF INLVSRYLSG TEATHVEWSK IQTPTDEVVV | 60 |
| PYDTLAPTPE DPAETKKLLD KLVVLKLNGG LGTTMGCTGP KSVIEVRNGL TFLDLIVIQI | 120 |
| ETLNNKYGCN VPLLLMNSFN THDDTFKIVE RYTKSNVQIH TFNQSQYPRL VVEDNSPLPS | 180 |
| KGQTGKDGWY PPGHGDVFPS LRNSGKLDLL LSQGKEYVFI SNSDNLGAVV DLKILSHLVQ | 240 |
| KKNEYCMEVT PKTLADVKGG TLISYEGRTQ LLEIAQVPDQ HVNEFKSIEK FKIFNTNNLW | 300 |
| VNLNAIKRLV EADALKMEII PNPKEVDGIK VLQLETAAGA AIRFFNHAIG INVPRSRFLP | 360 |
| VKATSDLLLV QSDLYTVEDG FVIRNTARKN PANPSVELGP EFKKVANFLS RFKSIPSIIE | 420 |
| LDSLKVVGDV WFGAGVVLKG KVTITAKPGV KLEIPDKAVL ENKDINGPED L | 471 |

SEQ ID NO: 134
H. vulgare

| | |
|---|---|
| atggctgctg ctgcagttgc tgctgattct aaaattgatg gtttgagaga tgctgttgcc | 60 |
| aagttgggtg aaatttctga aaacgaaaag gccggtttca tctccttggt ttctagatat | 120 |
| ttgtctggtg aagccgaaca aatcgaatgg tctaaaattc aaactccaac cgatgaagtt | 180 |
| gttgttccat atgatacttt ggctccacca cctgaagatt tggatgctat gaaggctttg | 240 |
| ttggataagt tggttgtctt gaagttgaat ggtggtttgg gtactactat gggttgtact | 300 |
| ggtccaaagt ctgttatcga agttagaaac ggtttcacct tcttggattt gatcgttatc | 360 |
| caaattgaat ccttgaacaa gaagtacggt tgctctgttc ctttgttgtt gatgaactct | 420 |
| ttcaacaccc atgatgacac ccaaaagatc gttgaaaagt actccaactc caacatcgaa | 480 |
| atccacacct tcaatcaatc tcaataccca agaatcgtca ccgaagattt tttgccattg | 540 |
| ccatctaaag gtcaaactgg taaagatggt tggtatccac caggtcatgg tgatgttttt | 600 |
| ccatctttga acaactccgg taagttggat accttgttgt ctcaaggtaa agaatacgtt | 660 |
| ttcgttgcca actctgataa cttgggtgct atcgttgata ttaagatctt gaaccacttg | 720 |
| atccacaatc aaaacgaata ctgcatggaa gttactccaa agactttggc tgatgttaag | 780 |
| ggtggtactt tgatttctta cgaaggtaga gttcaattat tggaaatcgc ccaagttcca | 840 |
| gatgaacacg ttgatgaatt caagtccatc gaaaagttca aaatcttcaa caccaacaac | 900 |
| ttgtgggtta acttgaaggc cattaagaga ttggttgatg ctgaagcttt gaaaatggaa | 960 |
| atcatcccaa accctaaaga agttgacggt gttaaggtat tgcaattgga aactgctgct | 1020 |
| ggtgctgcta ttagattctt tgaaaaagcc atcggtatca acgtcccaag atctagattt | 1080 |
| ttgccagtta aggctacctc tgacttgttg ttggttcaat cagacttgta caccttggtt | 1140 |
| gacggttacg ttattagaaa tccagctaga gttaagccat ccaacccatc tattgaattg | 1200 |
| ggtccagaat tcaagaaggt cgctaatttc ttggctagat tcaagtctat cccatccatc | 1260 |
| gttgaattgg actcattgaa agtttctggt gatgtctctt ttggttccgg tgttgttttg | 1320 |
| aagggtaatg ttactattgc tgctaaggct ggtgttaagt tggaaattcc agatggtgct | 1380 |
| gttttggaaa acaaggatat taacggtcca gaagatattt ga | 1422 |

SEQ ID NO: 135
H. vulgare

| | |
|---|---|
| MAAAAVAADS KIDGLRDAVA KLGEISENEK AGFISLVSRY LSGEAEQIEW SKIQTPTDEV | 60 |
| VVPYDTLAPP PEDLDAMKAL LDKLVVLKLN GGLGTTMGCT GPKSVIEVRN GFTFLDLIVI | 120 |
| QIESLNKKYG CSVPLLLMNS FNTHDDTQKI VEKYSNSNIE IHTFNQSQYP RIVTEDFLPL | 180 |
| PSKGQTGKDG WYPPGHGDVF PSLNNSGKLD TLLSQGKEYV FVANSDNLGA IVDIKILNHL | 240 |
| IHNQNEYCME VTPKTLADVK GGTLISYEGR VQLLEIAQVP DEHVDEFKSI EKFKIFNTNN | 300 |
| LWVNLKAIKR LVDAEALKME IIPNPKEVDG VKVLQLETAA GAAIRFFEKA IGINVPRSRF | 360 |
| LPVKATSDLL LVQSDLYTLV DGYVIRNPAR VKPSNPSIEL GPEFKKVANF LARFKSIPSI | 420 |
| VELDSLKVSG DVSFGSGVVL KGNVTIAAKA GVKLEIPDGA VLENKDINGP EDI | 473 |

SEQ ID NO: 136
O. sativa

| | |
|---|---|
| atggctgacg aaaaattggc caaattgaga gaagctgttg ctggtttgtc tcaaatctct | 60 |
| gataacgaaa agtccggttt catttccttg gttgctagat atttgtccgg tgaagaagaa | 120 |
| catgttgaat gggctaaaat tcatacccca accgatgaag ttgttgttcc atatgatact | 180 |
| ttggaagctc caccagaaga tttggaagaa acaaaaaagt tgttgaacaa gttggccgtc | 240 |
| ttgaagttga atggtggttt gggtactact atgggttgta ctggtccaaa gtctgttatc | 300 |
| gaagttagaa acggtttcac cttcttggat ttgatcgtca tccaaatcga atccttgaac | 360 |
| aaaaagtacg gttccaacgt tcctttgttg ttgatgaact ctttcaacac ccatgaagat | 420 |
| accttgaaga tcgttgaaaa gtacaccaac tccaacatcg aagttcacac cttcaatcaa | 480 |
| tctcaatacc caagagttgt tgccgatgaa tttttgccat ggccatctaa aggtaagact | 540 |
| tgtaaagatg gttggtatcc accaggtcat ggtgatattt ttccatcctt gatgaacagt | 600 |
| ggtaagttgg acttgttgtt gtcccaaggt aaagaatacg ttttcattgc caactccgat | 660 |
| aacttgggtg ctatagttga tatgaagatt ttgaaccact tgatccacaa gcaaaacgaa | 720 |
| tactgtatgg aagttactcc aaagactttg gctgatgtta agggtggtac tttgatctct | 780 |
| tacgaagata aggttcaatt attggaaatc gcccaagttc cagatgctca tgttaatgaa | 840 |
| ttcaagtcca tcgaaaagtt caagatcttt aacaccaaca acttgtgggt taacttgaag | 900 |
| gccattaaga gattagttga agctgacgct ttgaagatgg aaattatccc aaacccaaaa | 960 |
| gaagttgacg gtgttaaggt attgcaattg gaaactgctg ctggtgctgc tattagattt | 1020 |
| ttcgatcatg ctatcggtat caacgtccca agatctagat ttttaccagt taaggctacc | 1080 |
| tccgacttgc aattagttca atctgacttg tacaccttgg ttgatggttt cgttactaga | 1140 |
| aatccagcta gaactaatcc atccaaccca tctattgaat tgggtccaga attcaagaag | 1200 |
| gttggttgtt ttttgggtag attcaagtct atcccatcca tcgttgaatt ggacactttg | 1260 |
| aaagtttctg gtgatgtttg gttcggttcc tccattacat tgaaaggtaa ggttactatt | 1320 |
| accgctcaac caggtgttaa gttggaaatt ccagatggtg ctgtcatcga aaacaaggat | 1380 |
| attaacggtc ctgaagattt gtga | 1404 |

SEQ ID NO: 137
O. sativa

| | |
|---|---|
| MADEKLAKLR EAVAGLSQIS DNEKSGFISL VARYLSGEEE HVEWAKIHTP TDEVVVPYDT | 60 |
| LEAPPEDLEE TKKLLNKLAV LKLNGGLGTT MGCTGPKSVI EVRNGFTFLD LIVIQIESLN | 120 |
| KKYGSNVPLL LMNSFNTHED TLKIVEKYTN SNIEVHTFNQ SQYPRVVADE FLPWPSKGKT | 180 |
| CKDGWYPPGH GDIFPSLMNS GKLDLLLSQG KEYVFIANSD NLGAIVDMKI LNHLIHKQNE | 240 |
| YCMEVTPKTL ADVKGGTLIS YEDKVQLLEI AQVPDAHVNE FKSIEKFKIF NTNNLWVNLK | 300 |
| AIKRLVEADA LKMEIIPNPK EVDGVKVLQL ETAAGAAIRF FDHAIGINVP RSRFLPVKAT | 360 |
| SDLQLVQSDL YTLVDGFVTR NPARTNPSNP SIELGPEFKK VGCFLGRFKS IPSIVELDTL | 420 |
| KVSGDVWFGS SITLKGKVTI TAQPGVKLEI PDGAVIENKD INGPEDL | 467 |

SEQ ID NO: 138
S. tuberosum

| | |
|---|---|
| atggctactg ctactacttt gtctccagct gatgctgaaa agttgaacaa tttgaaatct | 60 |
| gctgtcgccg gtttgaatca aatctctgaa aacgaaaagt ccggtttcat caacttggtt | 120 |
| ggtagatatt tgtctggtga agcccaacat attgactggt ctaaaattca aactccaacc | 180 |
| gatgaagttg ttgtcccata tgataagttg gctccattgt ctgaagatcc agctgaaaca | 240 |
| aaaaagttgt tggacaagtt ggtcgtcttg aagttgaatg gtggtttggg tactactatg | 300 |
| ggttgtactg gtccaaagtc tgttatcgaa gttagaaacg gtttgacctt cttggatttg | 360 |
| atcgtcaagc aaattgaagc tttgaacgct aagttcggtt gttctgttcc tttgttgttg | 420 |
| atgaactctt tcaacaccca tgatgacacc ttgaagatcg ttgaaaagta cgccaactcc | 480 |
| aacattgata tccacacctt caatcaatcc caatacccaa gattggttac cgaagatttt | 540 |
| gctccattgc catgtaaagg taactctggt aaagatggtt ggtatccacc aggtcatggt | 600 |
| gatgtttttc catccttgat gaattccggt aagttggatg ctttgttggc taagggtaaa | 660 |
| gaatacgttt tcgttgccaa ctctgataac ttgggtgcta tcgttgattt gaaaatcttg | 720 |
| aaccacttga tcttgaacaa gaacgaatac tgcatggaag ttactccaaa gactttggct | 780 |
| gatgttaagg gtggtacttt gatttcttac gaaggtaagg ttcaattatt ggaaatcgcc | 840 |
| caagttccag atgaacacgt taatgaattc aagtccatcg aaaagtttaa gatcttcaac | 900 |
| actaacaact tgtgggtcaa cttgtctgcc attaagagat tggttgaagc tgatgccttg | 960 |
| aaaatggaaa ttattccaaa cccaaaagaa gtcgatggtg tcaaagtatt gcaattggaa | 1020 |
| actgctgctg gtgctgctat taagtttttc gatagagcta ttggtgccaa cgttccaaga | 1080 |
| tctagatttt tgccagttaa ggctacctct gacttgttgt tggttcaatc agacttgtac | 1140 |
| actttgactg atgaaggtta cgttattaga aacccagcta gatccaatcc atccaaccca | 1200 |
| tctattgaat tgggtccaga attcaagaag gtagccaatt ttttgggtag attcaagtct | 1260 |
| atcccatcca tcatcgattt ggattctttg aaagttactg gtgatgtctg gtttggttct | 1320 |
| ggtgttactt tgaaaggtaa agttaccgtt gctgctaagt caggtgttaa gttggaaatt | 1380 |
| ccagatggtg ctgttattgc caacaaggat attaacggtc cagaagatat ctaa | 1434 |

SEQ ID NO: 139
S. tuberosum

| | |
|---|---|
| MATATTLSPA DAEKLNNLKS AVAGLNQISE NEKSGFINLV GRYLSGEAQH IDWSKIQTPT | 60 |
| DEVVVPYDKL APLSEDPAET KKLLDKLVVL KLNGGLGTTM GCTGPKSVIE VRNGLTFLDL | 120 |
| IVKQIEALNA KFGCSVPLLL MNSFNTHDDT LKIVEKYANS NIDIHTFNQS QYPRLVTEDF | 180 |
| APLPCKGNSG KDGWYPPGHG DVFPSLMNSG KLDALLAKGK EYVFVANSDN LGAIVDLKIL | 240 |
| NHLILNKNEY CMEVTPKTLA DVKGGTLISY EGKVQLLEIA QVPDEHVNEF KSIEKFKIFN | 300 |
| TNNLWVNLSA IKRLVEADAL KMEIIPNPKE VDGVKVLQLE TAAGAAIKFF DRAIGANVPR | 360 |
| SRFLPVKATS DLLLVQSDLY TLTDEGYVIR NPARSNPSNP SIELGPEFKK VANFLGRFKS | 420 |
| IPSIIDLDSL KVTGDVWFGS GVTLKGKVTV AAKSGVKLEI PDGAVIANKD INGPEDI | 477 |

SEQ ID NO: 140
A. thaliana

| | |
|---|---|
| atgttcttgt tggttacctc ttgcttcttg ccagattctg gttcttctgt taaggtcagt | 60 |
| ttgttcatct tcggtgtctc attggtttct acctctccaa ttgatggtca aaaaccaggt | 120 |
| acttctggtt tgagaaagaa ggtcaaggtt ttcaagcaac ctaactactt ggaaaacttc | 180 |
| gttcaagcta ctttcaacgc tttgactacc gaaaaagtta agggtgctac tttggttgtt | 240 |
| tctggtgatg gtagatatta ctccgaacaa gccattcaaa tcatcgttaa gatggctgct | 300 |
| gctaacggtg ttagaagagt ttgggttggt caaaactctt tgttgtctac tccagctgtt | 360 |
| tccgccatta ttagagaaag agttggtgct gatggttcta aagctactgg tgctttcatt | 420 |
| ttgactgctt ctcataatcc aggtggtcca actgaagatt tcggtattaa gtacaacatg | 480 |
| gaaaatggtg gtccagcccc agaatctatt actgataaga tatacgaaaa caccaagacc | 540 |
| atcaaagaat acccaattgc agaagatttg ccaagagttg atatctctac tatcggtatc | 600 |
| acttctttcg aaggtcctga aggtaaattc gacgttgaag tttttgattc cgctgatgat | 660 |
| tacgtcaagt tgatgaagtc catcttcgac ttcgaatcca tcaagaagtt gttgtcttac | 720 |
| ccaaagttca ccttttgtta cgatgcattg catggtgttg ctggtgctta tgctcataga | 780 |
| attttcgttg aagaattggg tgctccagaa tcctctttat tgaactgtgt tccaaaagaa | 840 |
| gattttggtg gtggtcatcc agatccaaat ttgacttatg ccaaagaatt ggttgccaga | 900 |
| atgggtttgt ctaagactga tgatgctggt ggtgaaccac ctgaatttgg tgctgctgca | 960 |
| gatggtgatg ctgatagaaa tatgatcttg ggtaaaagat tcttcgtcac cccatctgat | 1020 |
| tccgttgcta ttattgctgc taatgctgtt ggtgctattc catacttttc atccggtttg | 1080 |
| aaaggtgttg ctagatctat gccaacttct gctgctttgg atgttgttgc taagaatttg | 1140 |
| ggtttgaagt tcttcgaagt tccaactggt tggaaattct tcggtaattt gatggatgca | 1200 |
| ggtatgtgtt ctgtttgcgg tgaagaatca tttggtactg gttccgatca tatcagagaa | 1260 |
| aaggatggta tttgggctgt tttggcttgg ttgtctattt tggctcacaa gaacaaagaa | 1320 |
| accttggatg gtaatgccaa gttggttact gttgaagata tcgttagaca acattgggct | 1380 |
| acttacggta gacattacta cactagatac gactacgaaa acgttgatgc tacagctgct | 1440 |
| aaagaattga tgggtttatt ggtcaagttg caatcctcat tgccagaagt taacaagatc | 1500 |
| atcaagggta tccatcctga agttgctaat gttgcttctg ctgatgaatt cgaatacaag | 1560 |
| gatccagttg atggttccgt ttctaaacat caaggtatca gatacttgtt tgaagatggt | 1620 |
| tccagattgg ttttcagatt gtctggtaca ggttctgaag gtgctactat tagattgtac | 1680 |
| atcgaacaat acgaaaagga cgcctctaag attggtagag attctcaaga tgctttgggt | 1740 |
| ccattggttg atgttgcttt gaagttgtcc aagatgcaag aattcactgg tagatcttct | 1800 |
| ccaaccgtta ttacctga | 1818 |

SEQ ID NO: 141
A. thaliana

| | |
|---|---|
| MFLLVTSCFL PDSGSSVKVS LFIFGVSLVS TSPIDGQKPG TSGLRKKVKV FKQPNYLENF | 60 |
| VQATFNALTT EKVKGATLVV SGDGRYYSEQ AIQIIVKMAA ANGVRRVWVG QNSLLSTPAV | 120 |
| SAIIRERVGA DGSKATGAFI LTASHNPGGP TEDFGIKYNM ENGGPAPESI TDKIYENTKT | 180 |
| IKEYPIAEDL PRVDISTIGI TSFEGPEGKF DVEVFDSADD YVKLMKSIFD FESIKKLLSY | 240 |
| PKFTFCYDAL HGVAGAYAHR IFVEELGAPE SSLLNCVPKE DFGGGHPDPN LTYAKELVAR | 300 |
| MGLSKTDDAG GEPPEFGAAA DGDADRNMIL GKRFFVTPSD SVAIIAANAV GAIPYFSSGL | 360 |
| KGVARSMPTS AALDVVAKNL GLKFFEVPTG WKFFGNLMDA GMCSVCGEES FGTGSDHIRE | 420 |
| KDGIWAVLAW LSILAHKNKE TLDGNAKLVT VEDIVRQHWA TYGRHYYTRY DYENVDATAA | 480 |
| KELMGLLVKL QSSLPEVNKI IKGIHPEVAN VASADEFEYK DPVDGSVSKH QGIRYLFEDG | 540 |
| SRLVFRLSGT GSEGATIRLY IEQYEKDASK IGRDSQDALG PLVDVALKLS KMQEFTGRSS | 600 |
| PTVIT | 605 |

SEQ ID NO: 142
E. coli

| | |
|---|---|
| atggccattc ataatagagc tggtcaacca gcacaacaat ccgatttgat taacgttgct | 60 |
| caattgaccg cccaatatta cgttttgaaa cctgaagctg gtaacgctga acatgctgtt | 120 |
| aagtttggta cttctggtca tagaggttct gctgctagac attcttttaa cgaaccacat | 180 |
| attttggcta tcgctcaagc tattgctgaa gaaagagcta agaacggtat tactggtcca | 240 |
| tgttacgttg gtaaagatac ccatgctttg tctgaaccag ctttcatttc tgttttggaa | 300 |
| gttttggctg ctaacggtgt tgatgttatc gttcaagaaa acaacggttt cactccaact | 360 |
| ccagctgttt ctaatgctat tttggttcac aacaaaaagg gtggtccatt ggctgatggt | 420 |
| atagttatta ctccatctca taacccacct gaagatggtg gtattaagta caatccacca | 480 |
| aatggtggtc cagctgatac aaatgttact aaggttgttg aagatagagc caacgctttg | 540 |
| ttagctgatg gtttgaaagg tgtcaagaga atctctttgg atgaagctat ggcttcaggt | 600 |
| catgtcaaag aacaagattt ggttcaacca ttcgttgaag gtttggctga tatagttgat | 660 |
| atggctgcta ttcaaaaggc tggtttgact ttgggtgttg atccattggg tggttctggt | 720 |
| attgaatact ggaaaagaat cggtgaatat tacaacttga acttgaccat cgtcaacgat | 780 |
| caagttgacc aaactttcag attcatgcac ttggataagg atggtgctat tagaatggac | 840 |
| tgttcttctg aatgtgctat ggctggttta ttggctttga gagataagtt cgatttggct | 900 |
| tttgctaacg atccagatta cgatagacat ggtatcgtta ctccagcagg tttgatgaat | 960 |
| ccaaatcatt acttggctgt tgccatcaac tacttgtttc aacatagacc acaatggggt | 1020 |
| aaggatgttg ctgttggtaa aactttggtt tcctccgcta tgatcgatag agttgttaac | 1080 |
| gatttgggta gaaagttggt tgaagttcca gttggtttca agtggtttgt tgacggtttg | 1140 |
| tttgatggtt cttttggttt tggtggtgaa gaatctgctg gtgcttcatt tttgagattt | 1200 |
| gatggtactc catggtccac tgacaaagat ggtattatca tgtgtttgtt ggctgctgaa | 1260 |
| attactgctg ttactggtaa gaatccacaa gaacactaca acgaattggc taagagattt | 1320 |
| ggtgctccat cttacaatag attgcaagct gctgctactt ctgctcaaaa agctgcttta | 1380 |
| tctaagttgt ccccagaaat ggtttctgct tctactttag ctggtgatcc aattacagct | 1440 |
| agattgactg ctgctccagg taatggtgct tctattggtg gtttaaaggt tatgactgat | 1500 |
| aacggttggt ttgctgcaag accatctggt actgaagatg cttacaaaat ctactgcgaa | 1560 |
| tccttcttgg gtgaagaaca tagaaagcaa attgaaaaag aagccgtcga aatcgtcagt | 1620 |
| gaagttttga agaatgccta a | 1641 |

SEQ ID NO: 143
E. coli

| | |
|---|---|
| MAIHNRAGQP AQQSDLINVA QLTAQYYVLK PEAGNAEHAV KFGTSGHRGS AARHSFNEPH | 60 |
| ILAIAQAIAE ERAKNGITGP CYVGKDTHAL SEPAFISVLE VLAANGVDVI VQENNGFTPT | 120 |
| PAVSNAILVH NKKGGPLADG IVITPSHNPP EDGGIKYNPP NGGPADTNVT KVVEDRANAL | 180 |
| LADGLKGVKR ISLDEAMASG HVKEQDLVQP FVEGLADIVD MAAIQKAGLT LGVDPLGGSG | 240 |
| IEYWKRIGEY YNLNLTIVND QVDQTFRFMH LDKDGAIRMD CSSECAMAGL LALRDKFDLA | 300 |
| FANDPDYDRH GIVTPAGLMN PNHYLAVAIN YLFQHRPQWG KDVAVGKTLV SSAMIDRVVN | 360 |
| DLGRKLVEVP VGFKWFVDGL FDGSFGFGGE ESAGASFLRF DGTPWSTDKD GIIMCLLAAE | 420 |
| ITAVTGKNPQ EHYNELAKRF GAPSYNRLQA AATSAQKAAL SKLSPEMVSA STLAGDPITA | 480 |
| RLTAAPGNGA SIGGLKVMTD NGWFAARPSG TEDAYKIYCE SFLGEEHRKQ IEKEAVEIVS | 540 |
| EVLKNA | 546 |

SEQ ID NO: 144
R. suavissimus

| | |
|---|---|
| atgtcctccg gtaagattaa gagagttcaa actactccat tcgacggtca aaaaccaggt | 60 |
| acttctggtt tgagaaagaa ggttaaggtt ttcacccaac ctaactactt gcaaaacttc | 120 |
| gttcaatcta ccttcaacgc tttgccatct gataaggtaa aaggtgctag attggttgtt | 180 |
| tctggtgatg gtagatactt ctccaaagaa gccattcaaa tcatcattaa gatggctgct | 240 |
| ggtaacggtg ttaagtctgt ttgggttggt caaaatggtt tgttgtctac tccagctgtt | 300 |
| tctgctgttg ttagagaaag agttggtgct gatggttgta aagcttctgg tgctttcatt | 360 |
| ttgactgctt ctcataatcc aggtggtcca aatgaagatt tcggtatcaa gtacaacatg | 420 |
| gaaaatggtg gtccagctcc agaatctatt accaacaaaa tctacgaaaa caccacccaa | 480 |
| atcaaagaat acttgaccgt tgatttgcca gaagttgata ttactaagcc aggtgttact | 540 |
| accttcgaag ttgaaggtgg tactttcact gttgatgttt tcgattctgc ttccgattac | 600 |
| gtcaagttga tgaagtccat tttcgacttc gaatccatca gaaagttgtt gtcctctcca | 660 |
| aagttcacct tttgttttga tgcattgcat ggtgttggtg gtgcttacgc taaaagaatt | 720 |
| ttcgttgaag aattgggtgc caaagaatcc tctttgttga actgtgttcc taaagaagat | 780 |
| tttggtggtg gtcatccaga tccaaatttg acatatgcta aagaattggt cgccagaatg | 840 |
| ggtttgtcta agtctaatac tcaaaacgaa ccaccagaat ttggtgctgc tgcagatggt | 900 |
| gatgctgata gaaatatggt tttgggtaag agattcttcg ttaccccatc tgattccgtt | 960 |
| gctattattg ctgctaatgc tgttgaagct atcccatact tttctactgg tttgaaaggt | 1020 |
| gttgctagat ctatgccaac ttctgctgct ttggatgttg ttgctaaaca cttgaacttg | 1080 |
| aagttcttcg aagtaccaac tggttggaag tttttcggta atttgatgga tgctggtttg | 1140 |
| tgttctgttt gcggtgaaga atcttttggt actggttccg atcatatcag agaaaaggat | 1200 |
| ggtatttggg ctgttttggc ttggttgtca attattgcca tcaagaacaa ggataacatc | 1260 |
| ggtggtgata agttggttac cgttgaagat atcgttagaa aacattgggc tacttacggt | 1320 |
| agacattact acactagata cgattacgaa aacgttgatg ctggtaaggc taaagatttg | 1380 |
| atggcatcat tggtcaactt gcaatcatct ttgcctgaag ttaacaagat cgttaagggt | 1440 |
| atctgttccg atgttgcaaa tgttgttggt gccgatgaat tcgaatacaa ggattctgtt | 1500 |
| gatggttcca tctccaaaca tcaaggtatc agatacttgt tcgaagatgg ttcaagattg | 1560 |
| gttttcagat tgtctggtac aggttctgaa ggtgctacta ttagattgta catcgaacaa | 1620 |
| tacgaaaatg acccatccaa gatctccaga gaatcttctg aagctttggc tccattggtt | 1680 |
| gaagttgctt tgaaattgtc caagatgcaa gaattcactg gtagatcagc tccaactgtt | 1740 |
| attacctga | 1749 |

SEQ ID NO: 145
R. suavissimus

| | |
|---|---|
| MSSGKIKRVQ TTPFDGQKPG TSGLRKKVKV FTQPNYLQNF VQSTFNALPS DKVKGARLVV | 60 |
| SGDGRYFSKE AIQIIIKMAA GNGVKSVWVG QNGLLSTPAV SAVVRERVGA DGCKASGAFI | 120 |
| LTASHNPGGP NEDFGIKYNM ENGGPAPESI TNKIYENTTQ IKEYLTVDLP EVDITKPGVT | 180 |
| TFEVEGGTFT VDVFDSASDY VKLMKSIFDF ESIRKLLSSP KFTFCFDALH GVGGAYAKRI | 240 |
| FVEELGAKES SLLNCVPKED FGGGHPDPNL TYAKELVARM GLSKSNTQNE PPEFGAAADG | 300 |
| DADRNMVLGK RFFVTPSDSV AIIAANAVEA IPYFSTGLKG VARSMPTSAA LDVVAKHLNL | 360 |
| KFFEVPTGWK FFGNLMDAGL CSVCGEESFG TGSDHIREKD GIWAVLAWLS IIAIKNKDNI | 420 |
| GGDKLVTVED IVRKHWATYG RHYYTRYDYE NVDAGKAKDL MASLVNLQSS LPEVNKIVKG | 480 |
| ICSDVANVVG ADEFEYKDSV DGSISKHQGI RYLFEDGSRL VFRLSGTGSE GATIRLYIEQ | 540 |
| YENDPSKISR ESSEALAPLV EVALKLSKMQ EFTGRSAPTV IT | 582 |

SEQ ID NO: 146
S. rebaudiana

| | |
|---|---|
| atggcctctt tcaaggttaa cagagttgaa tcctctccaa tcgaaggtca aaaaccaggt | 60 |
| acttctggtt tgagaaagaa ggttaaggtt ttcacccaac cacattactt gcacaacttc | 120 |
| gttcaatcta ctttcaacgc tttgtctgcc gaaaaagtta agggttctac tttggttgtt | 180 |
| tccggtgatg gtagatatta ctccaaggat gccattcaaa tcatcattaa gatggctgct | 240 |
| gctaacggtg ttagaagagt ttgggttggt caaaatggtt tgttgtctac tccagctgtt | 300 |
| tctgctgttg ttagagaaag agttggtgct gatggttcta aatctaacgg tgctttcatt | 360 |
| ttgactgcct ctcataatcc aggtggtcca aatgaagatt tcggtatcaa gtacaacatg | 420 |
| gaaaatggtg gtccagctcc agaaggtatt actgataaga tttttgaaaa caccaagacc | 480 |
| atcaaagaat acttcattgc tgaaggtttg ccagacgttg atatttccgc tattggtatc | 540 |
| tcttcattct ctggtccaga tggtcaattc gatgttgatg ttttcgattc ctcttccgac | 600 |
| tacgtcaaat tgatgaagtc catcttcgac ttccaatcca tcaagaagtt gattacctcc | 660 |
| ccacaatttt ctttctgtta cgatgcttta catggtgttg gtggtgctta tgctaagcca | 720 |
| atttttgttg atgaattggg tgccaaagaa tcctctttgt tgaactgtgt tcctaaagaa | 780 |
| gattttggtg gtggtcatcc agatccaaat ttgacttacg ctaaagaatt ggtttccaga | 840 |
| atgggtttgg gtaagaatcc agattctaat ccaccagaat ttggtgctgc tgcagatggt | 900 |
| gatgctgata gaaatatgat cttgggtaaa agattcttcg tcaccccatc tgattccgtt | 960 |
| gctattattg ctgctaatgc cgttcaatca atcccatact tttcatccgg tttgaaaggt | 1020 |
| gttgctagat ctatgccaac ttctgctgct ttggatgttg ttgctaagtc tttgaacttg | 1080 |
| aagttcttcg aagttccaac tggttggaag tttttcggta atttgatgga tgctggtttg | 1140 |
| tgttctgttt gcggtgaaga atcatttggt actggttccg atcatatcag agaaaaggat | 1200 |
| ggtatttggg ctgttttggc ttggttgtct attttggctc ataagaacaa ggacaacttg | 1260 |
| aacggtggta acttggttac tgttgaagat atcgttaagc aacattgggc tacttacggt | 1320 |
| agacattact acactagata cgactacgaa aacgttgatg ctggtgctgc aaaagaattg | 1380 |
| atggctcatt tggttaagtt gcaatcctcc atctctgatg ttaacacctt cattaagggt | 1440 |
| atcagatccg atgttgctaa tgttgcatct gctgatgaat tcgaatacaa ggatccagtt | 1500 |
| gacggttcta tttccaaaca tcaaggtatt agatacttgt ttgaagatgg ttccagattg | 1560 |
| gttttcagat tgtctggtac aggttctgaa ggtgctacta ttagattgta catcgaacaa | 1620 |
| tacgaaaagg attcctctaa gaccggtaga gattctcaag aagctttggc tccattagtt | 1680 |
| gaagttgcct tgaaattgtc caagatgcaa gaattcactg gtagatctgc tccaactgtt | 1740 |
| attacctga | 1749 |

SEQ ID NO: 147
S. rebaudiana

| | |
|---|---|
| MASFKVNRVE SSPIEGQKPG TSGLRKKVKV FTQPHYLHNF VQSTFNALSA EKVKGSTLVV | 60 |
| SGDGRYYSKD AIQIIIKMAA ANGVRRVWVG QNGLLSTPAV SAVVRERVGA DGSKSNGAFI | 120 |
| LTASHNPGGP NEDFGIKYNM ENGGPAPEGI TDKIFENTKT IKEYFIAEGL PDVDISAIGI | 180 |
| SSFSGPDGQF DVDVFDSSSD YVKLMKSIFD FQSIKKLITS PQFSFCYDAL HGVGGAYAKP | 240 |
| IFVDELGAKE SSLLNCVPKE DFGGGHPDPN LTYAKELVSR MGLGKNPDSN PPEFGAAADG | 300 |
| DADRNMILGK RFFVTPSDSV AIIAANAVQS IPYFSSGLKG VARSMPTSAA LDVVAKSLNL | 360 |
| KFFEVPTGWK FFGNLMDAGL CSVCGEESFG TGSDHIREKD GIWAVLAWLS ILAHKNKDNL | 420 |
| NGGNLVTVED IVKQHWATYG RHYYTRYDYE NVDAGAAKEL MAHLVKLQSS ISDVNTFIKG | 480 |
| IRSDVANVAS ADEFEYKDPV DGSISKHQGI RYLFEDGSRL VFRLSGTGSE GATIRLYIEQ | 540 |
| YEKDSSKTGR DSQEALAPLV EVALKLSKMQ EFTGRSAPTV IT | 582 |

SEQ ID NO: 148
Artificial Sequence

| | |
|---|---|
| gcacacacca tagcttcaaa atgtttctac tcctttttta ctcttccaga ttttctcgga | 60 |
| ctccgcgcat cgccgtacca cttcaaaaca cccaagcaca gcatactaaa tttcccctct | 120 |
| ttcttcctct agggtgtcgt taattacccg tactaaaggt ttggaaaaga aaaaagagac | 180 |
| cgcctcgttt ctttttcttc gtcgaaaaag gcaataaaaa tttttatcac gtttcttttt | 240 |
| cttgaaaatt tttttttttg atttttttct ctttcgatga cctcccattg atatttaagt | 300 |
| taataaacgg tcttcaattt ctcaagtttc agtttcattt ttcttgttct attacaactt | 360 |
| tttttacttc ttgctcatta gaaagaaagc atagcaatct aatctaagtt ttaattacaa | 420 |
| ggatcc | 426 |

SEQ ID NO: 149
Artificial Sequence

| | |
|---|---|
| ggaagtacct tcaaagaatg gggtcttatc ttgttttgca agtaccactg agcaggataa | 60 |
| taatagaaat gataatatac tatagtagag ataacgtcga tgacttccca tactgtaatt | 120 |
| gcttttagtt gtgtattttt agtgtgcaag tttctgtaaa tcgattaatt tttttttctt | 180 |
| tcctcttttt attaacctta atttttattt tagattcctg acttcaactc aagacgcaca | 240 |
| gatattataa catctgcata ataggcattt gcaagaatta ctcgtgagta aggaaagagt | 300 |
| gaggaactat cgcatacctg catttaaaga tgccgatttg ggcgcgaatc ctttattttg | 360 |
| gcttcaccct catactatta tcagggccag aaaaaggaag tgtttccctc cttcttgaat | 420 |
| tgatgttacc ctcataaagc acgtggcctc ttatcgagaa agaaattacc gtcgctcgtg | 480 |
| atttgtttgc aaaaagaaca aaactgaaaa aacccagaca cgctcgactt cctgtcttcc | 540 |
| tattgattgc agcttccaat ttcgtcacac aacaaggtcc tagcgacggc tcacaggttt | 600 |
| tgtaacaagc aatcgaaggt tctggaatgg cgggaaaggg tttagtacca catgctatga | 660 |
| tgcccactgt gatctccaga gcaaagttcg ttcgatcgta ctgttactct ctctctttca | 720 |
| aacagaattg tccgaatcgt gtgacaacaa cagcctgttc tcacacactc ttttcttcta | 780 |
| accaaggggg tggtttagtt tagtagaacc tcgtgaaact tacatttaca tatatataaa | 840 |
| cttgcataaa ttggtcaatg caagaaatac atatttggtc ttttctaatt cgtagttttt | 900 |
| caagttctta gatgctttct ttttctcttt tttacagatc atcaaggaag taattatcta | 960 |
| ctttttacaa caaatataaa acaa | 984 |

SEQ ID NO: 150
Artificial Sequence

| | |
|---|---|
| cattatcaat actgccattt caaagaatac gtaaataatt aatagtagtg attttcctaa | 60 |
| ctttatttag tcaaaaaatt agccttttaa ttctgctgta acccgtacat gcccaaaata | 120 |
| gggggcgggt tacacagaat atataacatc gtaggtgtct gggtgaacag tttattcctg | 180 |
| gcatccacta aatataatgg agcccgcttt ttaagctggc atccagaaaa aaaaagaatc | 240 |
| ccagcaccaa aatattgttt tcttcaccaa ccatcagttc ataggtccat tctcttagcg | 300 |
| caactacaga gaacaggggc acaaacaggc aaaaaacggg cacaacctca atggagtgat | 360 |
| gcaacctgcc tggagtaaat gatgacacaa ggcaattgac ccacgcatgt atctatctca | 420 |
| ttttcttaca ccttctatta ccttctgctc tctctgattt ggaaaaagct gaaaaaaaag | 480 |
| gttgaaacca gttccctgaa attattcccc tacttgacta ataagtatat aaagacggta | 540 |
| ggtattgatt gtaattctgt aaatctattt cttaaacttc ttaaattcta cttttatagt | 600 |
| tagtcttttt tttagtttta aaacaccaag aacttagttt cgaataaaca cacataaaca | 660 |
| aacaaa | 666 |

SEQ ID NO: 151
Artificial Sequence

| | |
|---|---|
| gatctgggcc gtatacttac atatagtaga tgtcaagcgt aggcgcttcc cctgccggct | 60 |
| gtgagggcgc cataaccaag gtatctatag accgccaatc agcaaactac ctccgtacat | 120 |
| tcatgttgca cccacacatt tatacaccca gaccgcgaca aattacccat aaggttgttt | 180 |
| gtgacggcgt cgtacaagag aacgtgggaa ctttttaggc tcaccaaaaa agaaagaaaa | 240 |
| aatacgagtt gctgacagaa gcctcaagaa aaaaaaaatt cttcttcgac tatgctggag | 300 |
| gcagagatga tcgagccggt agttaactat atatagctaa attggttcca tcaccttctt | 360 |
| ttctggtgtc gctccttcta gtgctatttc tggcttttcc tatttttttt tttccatttt | 420 |
| tctttctctc tttctaatat ataaattctc ttgcattttc tatttttctc tctatctatt | 480 |
| ctacttgttt attcccttca aggttttttt ttaaggagta cttgttttta gaatatacgg | 540 |
| tcaacgaact ataattaact aaaca | 565 |

SEQ ID NO: 152
Artificial Sequence

| | |
|---|---|
| agttataata atcctacgtt agtgtgagcg ggatttaaac tgtgaggacc ttaatacatt | 60 |
| cagacacttc tgcggtatca ccctacttat tcccttcgag attatatcta ggaacccatc | 120 |
| aggttggtgg aagattaccc gttctaagac ttttcagctt cctctattga tgttacacct | 180 |
| ggacacccct tttctggcat ccagttttta atcttcagtg gcatgtgaga ttctccgaaa | 240 |
| ttaattaaag caatcacaca attctctcgg ataccacctc ggttgaaact gacaggtggt | 300 |
| ttgttacgca tgctaatgca aaggagccta tatacctttg gctcggctgc tgtaacaggg | 360 |
| aatataaagg gcagcataat ttaggagttt agtgaacttg caacatttac tattttccct | 420 |
| tcttacgtaa atatttttct ttttaattct aaatcaatct ttttcaattt tttgtttgta | 480 |
| ttcttttctt gcttaaatct ataactacaa aaaacacata cataaactaa aa | 532 |

SEQ ID NO: 153
Artificial Sequence

| | |
|---|---|
| gatctatgcg actgggtgag catatgttcc gctgatgtga tgtgcaagat aaacaagcaa | 60 |
| ggcagaaact aacttcttct tcatgtaata aacacacccc gcgtttattt acctatctct | 120 |
| aaacttcaac accttatatc ataactaata tttcttgaga taagcacact gcacccatac | 180 |
| cttccttaaa aacgtagctt ccagtttttg gtggttccgg cttccttccc gattccgccc | 240 |
| gctaaacgca tatttttgtt gcctggtggc atttgcaaaa tgcataacct atgcatttaa | 300 |
| aagattatgt atgctcttct gacttttcgt gtgatgaggc tcgtggaaaa aatgaataat | 360 |
| ttatgaattt gagaacaatt ttgtgttgtt acggtatttt actatggaat aatcaatcaa | 420 |
| ttgaggattt tatgcaaata tcgtttgaat atttttccga ccctttgagt acttttcttc | 480 |
| ataattgcat aatattgtcc gctgcccctt tttctgttag acggtgtctt gatctacttg | 540 |
| ctatcgttca acaccacctt attttctaac tatttttttt ttagctcatt tgaatcagct | 600 |
| tatggtgatg gcacattttt gcataaacct agctgtcctc gttgaacata ggaaaaaaaa | 660 |
| atatataaac aaggctcttt cactctcctt gcaatcagat ttgggtttgt tccctttatt | 720 |
| ttcatatttc ttgtcatatt cctttctcaa ttattatttt ctactcataa cctcacgcaa | 780 |
| aataacacag tcaaatctat caaaa | 805 |

SEQ ID NO: 154
Artificial Sequence

| | |
|---|---|
| atccgctcta accgaaaagg aaggagttag acaacctgaa gtctaggtcc ctatttattt | 60 |
| tttttaatag ttatgttagt attaagaacg ttatttatat ttcaaatttt tctttttttt | 120 |
| ctgtacaaac gcgtgtacgc atgtaacatt atactgaaaa ccttgcttga gaaggttttg | 180 |
| ggacgctcga ag | 192 |

SEQ ID NO: 155
Artificial Sequence

| | |
|---|---|
| gtagatacgt tgttgacact tctaaataag cgaatttctt atgatttatg atttttatta | 60 |
| ttaaataagt tataaaaaaa ataagtgtat acaaatttta aagtgactct taggttttaa | 120 |
| aacgaaaatt cttattcttg agtaactctt tcctgtaggt caggttgctt tctcaggtat | 180 |
| agcatgaggt cgctc | 195 |

SEQ ID NO: 156
S. cerevisiae

| | |
|---|---|
| atgaatagat cattactgct acgtttgtcg gataccggtg aacccattac aagctgctct | 60 |
| tacggaaaag gtgtcttgac gctaccacca attccgctcc ctaaggacgc cccaaaggac | 120 |
| caaccgctct atacggtcaa gctactggta tctgcaggtt cccctgtcgc tagggatggg | 180 |
| ctagtttgga ctaattgccc accagatcac aacacgccct tcaagaggga caaattttac | 240 |
| aaaaaaatca ttcattccag ctttcacgag gatgactgca ttgacctgaa tgtctacgct | 300 |
| ccaggctcgt actgctttta tctatctttc aggaacgata acgaaaaact tgagacaaca | 360 |
| aggaaatact actttgttgc cttgcccatg ctttatataa acgatcagtt cctacctttg | 420 |
| aattccatcg ctttacaaag tgttgtatcg aaatggctgg gctctgactg ggagcccatc | 480 |
| ctatcgaaaa ttgccgctaa aaactacaat atggtacatt tcacccctct acaggaaaga | 540 |
| ggcgagtcta actcgcctta ctctatatac gaccaattgc agttcgacca ggaacacttt | 600 |
| aagtctcctg aagacgtgaa aaatttagtt gagcatatac atcgcgattt aaacatgctt | 660 |
| tcattaacag atattgtttt taaccacaca gctaataatt ctccttggtt agttgagcac | 720 |
| ccggaggctg ggtataacca catcactgcg ccacatctaa tcagcgccat agagctcgac | 780 |
| caagaattgc tcaattttag taggaatttg aaatcctggg gctatcctac cgaactgaaa | 840 |
| aatatagaag atctcttcaa gatcatggac ggtattaaag tgcatgtttt agggtcgttg | 900 |
| aaactgtggg aatattatgc ggtaaacgtg caaacagctc ttcgggatat caaagcccat | 960 |
| tggaatgacg aatctaacga aagttacagt tttcccgaga atattaaaga catctcgtcc | 1020 |
| gatttcgtaa aactagcttc ctttgtgaag gacaacgtca ctgagcctaa cttcggcact | 1080 |
| cttggtgaaa gaaactcaaa caggattaac gtgccaaaat ttattcaact actgaagctc | 1140 |
| attaacgatg gtggtagtga tgacagtgaa tcttcgttgg ccacggctca aaacatcttg | 1200 |
| aacgaggtca acttaccctt atatagagaa tacgacgatg atgtcagtga gatactcgag | 1260 |
| caactgttca atcgtatcaa atatttgaga ttagatgacg gtgggcccaa gcaaggtcca | 1320 |
| gtgaccgttg acgtgccctt aacagagcct tattttacga ggttcaaagg aaaagatggt | 1380 |
| actgattatg ccctcgccaa caatggctgg atatggaatg gtaacccact agtggatttt | 1440 |
| gcatcgcaga attcaagagc ttatttacgt agagaagtta tcgtgtgggg ggactgtgtc | 1500 |
| aagttaagat acggtaaaag ccctgaagac tctccgtatc tgtgggaaag aatgtccaag | 1560 |
| tatatagaaa tgaacgccaa gatatttgac gggttcagaa ttgacaactg ccattctact | 1620 |
| ccaatacatg ttggcgaata tttcctagat ttggcaagaa aatacaaccc gaacctatat | 1680 |
| gtcgttgcag agctgttttc tggttccgaa acactagatt gtctgtttgt tgaacggttg | 1740 |
| ggtatctcct ctttaatcag agaggcaatg caagcctggt ccgaagaaga gttgtctaga | 1800 |
| ttagtccata agcatggcgg gaggcccatt ggctcctata agtttgttcc tatggatgac | 1860 |
| ttctcatatc ctgcggatat taatttaaac gaggagcatt gtttcaacga ctccaacgat | 1920 |
| aactccataa gatgtgtatc agagatcatg attccaaaga ttttaaccgc cactccgcca | 1980 |
| cacgctttat tcatggactg tacccatgat aatgaaactc cctttgaaaa aagaacagtg | 2040 |
| gaggatactt tgcccaatgc tgcattggtg gctctttgct cgtccgccat tggatctgtt | 2100 |
| tatggctacg acgaaatttt tccacattta ctgaatttgg tcactgaaaa aagacattat | 2160 |
| gacatttcta cgcctactgg tagcccctcg ataggaataa ccaaagtcaa ggccactttg | 2220 |
| aattcgatta gaacgagtat aggagaaaag gcgtatgaca ttgaagactc agaaatgcat | 2280 |
| gtgcatcacc agggccagta cattactttt catcgtatgg atgttaaatc cggaaaaggt | 2340 |
| tggtacttga tagcaaggat gaaattttct gacaatgatg accctaacga gactttacca | 2400 |
| ccagtggtgt taaaccaatc cacctgttct ctcaggtttt cgtatgcttt ggaaagagtt | 2460 |
| ggcgatgaaa ttcccaacga cgataaattc attaaaggta ttcccacgaa attaaaggag | 2520 |
| cttgaagggt ttgacatttc ttatgatgat tctaagaaga tttcaacgat aaaactgccc | 2580 |
| aatgaattcc ctcaaggatc tattgccatt tttgagaccc aacagaatgg tgtggacgaa | 2640 |
| tccttagatc attttataag gtcaggtgct ttaaaggcca cttcaagttt gactctagag | 2700 |
| tcaataaatt ccgtcttgta tcgtagtgag ccggaagaat acgatgttag cgccggcgaa | 2760 |
| ggtggtgctt atattattcc taattttgga aagcctgtgt attgtggtct gcaaggttgg | 2820 |
| gtttccgtat taagaaaaat tgtgttttac aatgatttag cacatcccct cagtgcaaat | 2880 |
| ttaagaaatg gacattgggc tttagactac actatcagta gacttaatta ctatagcgat | 2940 |
| gaagcaggaa tcaatgaagt gcagaactgg ctgcgttcaa ggtttgatag agtgaaaaag | 3000 |
| ttaccgagct acttagtgcc cagttatttc gccttaatta tcggcatcct ctatggttgt | 3060 |
| tgtcgcttaa aagcaataca gctaatgtcc cgtaatattg gtaaatctac attgtttgta | 3120 |
| caaagcttat ctatgacatc aatccagatg gtttccagaa tgaagtcaac ctctatttta | 3180 |
| ccaggcgaaa atgttccatc tatggctgca gggttgccac actttagcgt aaactacatg | 3240 |
| agatgttggg ggagagatgt attcatatcg ctaagaggta tgctattaac aacaggtaga | 3300 |
| tttgatgaag ctaaagctca tatactagcc tttgcaaaga ctttgaagca tggtttaatt | 3360 |
| ccaaacttgc tggatgccgg tagaaacccg agatataatg ctcgtgatgc tgcctggttc | 3420 |
| ttcttgcaag ctgtacagga ttatgtttat attgttcctg atggcgaaaa aatattacaa | 3480 |
| gagcaagtaa caaggagatt cccactggat gatacttaca ttcctgtaga tgatccaagg | 3540 |
| gcatttagtt actctagtac cttggaggag atcatttatg aaattttgag taggcatgcc | 3600 |
| aagggaatta aattcagaga ggctaatgca ggtccaaatt tagatcgtgt tatgactgat | 3660 |
| aaagggttta atgttgaaat tcatgtcgat tggtcgactg gcttaattca tggtggatct | 3720 |
| cagtataact gtggtacttg gatggataag atgggtgaaa gtgaaaaagc agggtctgtt | 3780 |
| ggtattcctg gaacacccag agatggagcc gcaatagaaa tcaatgggct tttaaaaagt | 3840 |
| gctttaaggt ttgttattga actaaaaaac aagggattgt ttaagttttc cgatgtggag | 3900 |
| acgcaggacg gcgggaggat cgatttcact gaatggaatc aattacttca agacaatttc | 3960 |
| gaaaaaagat attatgttcc ggaggatcca tcacaggatg cagattatga cgtgagcgct | 4020 |
| aaattgggtg ttaatagacg ggggatatac agagatttgt acaaatcagg aaagccttat | 4080 |
| gaagattatc agttaagacc aaattttgct attgccatga ctgtggcacc agagttattt | 4140 |
| gtgcctgagc atgccataaa agcaatcacc attgcagatg aagtcttaag aggtccagta | 4200 |
| ggtatgcgta ctttagaccc aagcgattac aattaccgtc cgtactacaa caacggagaa | 4260 |
| gattcggatg attttgccac ctcaaagggt agaaactatc accaaggccc tgagtgggtc | 4320 |
| tggctttacg gctacttttt aagagcgttc catcatttcc actttaaaac cagtccacgt | 4380 |
| tgtcagaatg ctgccaaaga gaaaccatcc tcttatttgt atcaacaatt atactacaga | 4440 |
| ttaaaaggcc atagaaaatg gatttttgaa agtgtgtggg caggattgac agagctaacc | 4500 |
| aataaagatg gtgaagtatg caatgactca agccccacgc aagcctggag ttctgcttgt | 4560 |
| ttgttagatc tattttatga tttatgggat gcctacgaag atgattcctg a | 4611 |

SEQ ID NO: 157
S. cerevisiae

| | |
|---|---|
| MNRSLLLRLS DTGEPITSCS YGKGVLTLPP IPLPKDAPKD QPLYTVKLLV SAGSPVARDG | 60 |
| LVWTNCPPDH NTPFKRDKFY KKIIHSSFHE DDCIDLNVYA PGSYCFYLSF RNDNEKLETT | 120 |
| RKYYFVALPM LYINDQFLPL NSIALQSVVS KWLGSDWEPI LSKIAAKNYN MVHFTPLQER | 180 |
| GESNSPYSIY DQLQFDQEHF KSPEDVKNLV EHIHRDLNML SLTDIVFNHT ANNSPWLVEH | 240 |
| PEAGYNHITA PHLISAIELD QELLNFSRNL KSWGYPTELK NIEDLFKIMD GIKVHVLGSL | 300 |
| KLWEYYAVNV QTALRDIKAH WNDESNESYS FPENIKDISS DFVKLASFVK DNVTEPNFGT | 360 |
| LGERNSNRIN VPKFIQLLKL INDGGSDDSE SSLATAQNIL NEVNLPLYRE YDDDVSEILE | 420 |
| QLFNRIKYLR LDDGGPKQGP VTVDVPLTEP YFTRFKGKDG TDYALANNGW IWNGNPLVDF | 480 |
| ASQNSRAYLR REVIVWGDCV KLRYGKSPED SPYLWERMSK YIEMNAKIFD GFRIDNCHST | 540 |
| PIHVGEYFLD LARKYNPNLY VVAELFSGSE TLDCLFVERL GISSLIREAM QAWSEEELSR | 600 |
| LVHKHGGRPI GSYKFVPMDD FSYPADINLN EEHCFNDSND NSIRCVSEIM IPKILTATPP | 660 |
| HALFMDCTHD NETPFEKRTV EDTLPNAALV ALCSSAIGSV YGYDEIFPHL LNLVTEKRHY | 720 |
| DISTPTGSPS IGITKVKATL NSIRTSIGEK AYDIEDSEMH VHHQGQYITF HRMDVKSGKG | 780 |
| WYLIARMKFS DNDDPNETLP PVVLNQSTCS LRFSYALERV GDEIPNDDKF IKGIPTKLKE | 840 |
| LEGFDISYDD SKKISTIKLP NEFPQGSIAI FETQQNGVDE SLDHFIRSGA LKATSSLTLE | 900 |
| SINSVLYRSE PEEYDVSAGE GGAYIIPNFG KPVYCGLQGW VSVLRKIVFY NDLAHPLSAN | 960 |
| LRNGHWALDY TISRLNYYSD EAGINEVQNW LRSRFDRVKK LPSYLVPSYF ALIIGILYGC | 1020 |
| CRLKAIQLMS RNIGKSTLFV QSLSMTSIQM VSRMKSTSIL PGENVPSMAA GLPHFSVNYM | 1080 |
| RCWGRDVFIS LRGMLLTTGR FDEAKAHILA FAKTLKHGLI PNLLDAGRNP RYNARDAAWF | 1140 |
| FLQAVQDYVY IVPDGEKILQ EQVTRRFPLD DTYIPVDDPR AFSYSSTLEE IIYEILSRHA | 1200 |
| KGIKFREANA GPNLDRVMTD KGFNVEIHVD WSTGLIHGGS QYNCGTWMDK MGESEKAGSV | 1260 |
| GIPGTPRDGA AIEINGLLKS ALRFVIELKN KGLFKFSDVE TQDGGRIDFT EWNQLLQDNF | 1320 |
| EKRYYVPEDP SQDADYDVSA KLGVNRRGIY RDLYKSGKPY EDYQLRPNFA IAMTVAPELF | 1380 |
| VPEHAIKAIT IADEVLRGPV GMRTLDPSDY NYRPYYNNGE DSDDFATSKG RNYHQGPEWV | 1440 |
| WLYGYFLRAF HHFHFKTSPR CQNAAKEKPS SYLYQQLYYR LKGHRKWIFE SVWAGLTELT | 1500 |
| NKDGEVCNDS SPTQAWSSAC LLDLFYDLWD AYEDDS | 1536 |

SEQ ID NO: 158
S. cerevisiae

| | |
|---|---|
| atgccgccag ctagtactag tactaccaat gatatgataa ccgaagaacc tacttctcca | 60 |
| caccaaatcc caaggcttac aaggagactt acggggtttc ttccccaaga aatcaagtca | 120 |
| attgacacga tgattccttt aaagtcaaga gcgttatgga ataagcatca agtcaaaaaa | 180 |
| tttaacaagg cagaagattt tcaagataga ttcattgacc atgtggaaac tacattagca | 240 |
| cgttccctat ataattgtga tgacatggct gcttatgaag ctgcttcgat gagtattcgt | 300 |
| gacaatttgg tcattgactg gaacaaaact cagcagaaat tcaccacaag agacccaaag | 360 |
| agagtttact atttgtcttt ggagtttttg atgggtaggg ctttggataa tgccctgatt | 420 |
| aatatgaaga ttgaagatcc ggaagaccct gctgcctcaa agggaaaacc aagagaaatg | 480 |
| attaaagggg ctttggatga tttaggtttc aagttagagg atgtcttgga ccaagaaccg | 540 |
| gacgcaggtt taggtaatgg tggtctaggt cgtcttgcag cttgcttcgt cgactcaatg | 600 |
| gcaacggaag gcatccctgc ctggggttat ggtctacgtt atgagtatgg tatctttgct | 660 |
| caaaagatta ttgacggtta ccaggtggaa actccagatt actggttaaa ttctggtaat | 720 |
| ccatgggaaa ttgaacgtaa cgaagtgcaa attcctgtca ccttttatgg ttatgttgat | 780 |
| agaccagaag gcggtaaaac tacactgagt gcgtcacaat ggatcggtgg ggaaagagtt | 840 |
| cttgctgtcg cgtatgattt cccagttccg ggtttcaaga cttccaatgt aaataactta | 900 |
| agactatggc aagcaaggcc aacaacagaa tttgattttg caaaattcaa taatggtgac | 960 |
| tataaaaact ctgtggctca gcaacaacgc gcagagtcta taaccgctgt gttgtatcca | 1020 |
| aacgataact ttgctcaagg taaggagttg aggttgaaac agcagtactt ctggtgtgct | 1080 |
| gcatccttac acgacatctt aagaagattc aaaaaatcca agaggccatg gactgaattt | 1140 |
| cctgaccaag tggctattca gttgaatgat actcatccaa ctttagccat cgttgaatta | 1200 |
| cagagagttt tggtcgatct agaaaaacta gattggcacg aggcttggga catcgtgacc | 1260 |
| aagacttttg cttatactaa ccacactgtt atgcaagagg ccctggaaaa atggcccgtc | 1320 |
| ggcctctttg gccatttgct acccagacat ttggaaatta tatatgatat caactggttc | 1380 |
| ttcttgcaag atgtggccaa aaaattcccc aaggatgttg atcttttgtc tcgtatatcc | 1440 |
| atcatcgaag aaaactctcc agaaagacag atcagaatgg cctttttggc tattgttggt | 1500 |
| tcacacaagg ttaatggtgt tgctgaattg cactctgaat taatcaaaac gaccatattt | 1560 |
| aaagattttg tcaagttcta tggtccatca aagtttgtca atgtcactaa cggtatcaca | 1620 |
| ccaaggagat ggttgaagca agctaaccct tcattggcta aactgatcag tgaaaccctt | 1680 |
| aacgatccaa cagaggagta tttgttggac atggccaaac tgacccagtt gggaaaatat | 1740 |
| gttgaagata aggagttttt gaaaaaatgg aaccaagtca agcttaataa taagatcaga | 1800 |
| ttagtagatt taatcaaaaa ggaaaatgat ggagtagaca tcattaacag agagtatttg | 1860 |
| gacgacacct tgtttgatat gcaagttaaa cgtattcatg aatataagcg tcaacagcta | 1920 |
| aacgtctttg gtattatata ccgttacctg gcaatgaaga atatgctgaa gaacggtgct | 1980 |
| tcgatcgaag aagttgccaa gaaatatcca cgcaaggttt caatctttgg tggtaagagt | 2040 |
| gctcctggtt actacatggc taagctgatc ataaaattga tcaactgtgt tgctgacatt | 2100 |
| gttaataacg acgagtcaat tgagcatttg ttgaaggttg tctttgttgc tgattataat | 2160 |
| gtttctaagg ctgaaatcat tattccagca agtgacttga gtgagcatat ttctactgct | 2220 |
| ggtactgaag cgtctggtac ttctaatatg aagtttgtta tgaacggtgg tttgattatt | 2280 |
| ggtactgttg atggtgccaa tgtggaaatc acaagggaaa ttggtgaaga taatgtcttc | 2340 |
| ttgtttggta acctaagtga aaatgtcgaa gaattgagat acaaccatca ataccatcca | 2400 |
| caagatttac catctagttt ggattctgtt ttatcctaca ttgaaagtgg acaattttct | 2460 |
| ccagaaaatc caaatgaatt caaaccttta gtcgacagta ttaagtacca cggcgattat | 2520 |
| tacctggtca gtgatgactt tgaatcctat ctggccaccc atgaattagt ggaccaggag | 2580 |
| ttccacaatc aaaggtcaga atggttaaaa aagagtgtcc tgagcgttgc aaacgtcggc | 2640 |
| ttctttagca gtgatcgttg tatcgaggaa tactccgata ccatttggaa cgttgaacca | 2700 |
| gtgacttag | 2709 |

SEQ ID NO: 159
S. cerevisiae

| | |
|---|---|
| MPPASTSTTN DMITEEPTSP HQIPRLTRRL TGFLPQEIKS IDTMIPLKSR ALWNKHQVKK | 60 |
| FNKAEDFQDR FIDHVETTLA RSLYNCDDMA AYEAASMSIR DNLVIDWNKT QQKFTTRDPK | 120 |
| RVYYLSLEFL MGRALDNALI NMKIEDPEDP AASKGKPREM IKGALDDLGF KLEDVLDQEP | 180 |
| DAGLGNGGLG RLAACFVDSM ATEGIPAWGY GLRYEYGIFA QKIIDGYQVE TPDYWLNSGN | 240 |
| PWEIERNEVQ IPVTFYGYVD RPEGGKTTLS ASQWIGGERV LAVAYDFPVP GFKTSNVNNL | 300 |
| RLWQARPTTE FDFAKFNNGD YKNSVAQQQR AESITAVLYP NDNFAQGKEL RLKQQYFWCA | 360 |
| ASLHDILRRF KKSKRPWTEF PDQVAIQLND THPTLAIVEL QRVLVDLEKL DWHEAWDIVT | 420 |
| KTFAYTNHTV MQEALEKWPV GLFGHLLPRH LEIIYDINWF FLQDVAKKFP KDVDLLSRIS | 480 |
| IIEENSPERQ IRMAFLAIVG SHKVNGVAEL HSELIKTTIF KDFVKFYGPS KFVNVTNGIT | 540 |
| PRRWLKQANP SLAKLISETL NDPTEEYLLD MAKLTQLGKY VEDKEFLKKW NQVKLNNKIR | 600 |
| LVDLIKKEND GVDIINREYL DDTLFDMQVK RIHEYKRQQL NVFGIIYRYL AMKNMLKNGA | 660 |
| SIEEVAKKYP RKVSIFGGKS APGYYMAKLI IKLINCVADI VNNDESIEHL LKVVFVADYN | 720 |
| VSKAEIIIPA SDLSEHISTA GTEASGTSNM KFVMNGGLII GTVDGANVEI TREIGEDNVF | 780 |
| LFGNLSENVE ELRYNHQYHP QDLPSSLDSV LSYIESGQFS PENPNEFKPL VDSIKYHGDY | 840 |
| YLVSDDFESY LATHELVDQE FHNQRSEWLK KSVLSVANVG FFSSDRCIEE YSDTIWNVEP | 900 |
| VT | 902 |

SEQ ID NO: 160
S. cerevisiae

| | |
|---|---|
| MSKQFSHTTN DRRSSIIYST SVGKAGLFTP ADYIPQESEE NLIEGEEQEG SEEEPSYTGN | 60 |
| DDETEREGEY HSLLDANNSR TLQQEAWQQG YDSHDRKRLL DEERDLLIDN KLLSQHGNGG | 120 |
| GDIESHGHGQ AIGPDEEERP AEIANTWESA IESGQKISTT FKRETQVITM NALPLIFTFI | 180 |
| LQNSLSLASI FSVAHLGTKE LGGVTLGSMT ANITGLAAIQ GLCTCLGTLC AQAYGAKNYH | 240 |
| LVGVLVQRCA VITILAFLPM MYVWFVWSEK ILALMIPERE LCALAANYLR VTAFGVPGFI | 300 |
| LFECGKRFLQ CQGIFHASTI VLFVCAPLNA LMNYLLVWND KIGIGYLGAP LSVVINYWLM | 360 |
| TLGLLIYAMT TKHKERPLKC WNGIIPKEQA FKNWRKMINL AIPGVVMVEA EFLGFEVLTI | 420 |
| FASHLGTDAL GAQSIVATIA SLAYQVPFSI SVSTSTRVAN FIGASLYDSC MITCRVSLLL | 480 |
| SFVCSSMNMF VICRYKEQIA SLFSTESAVV KMVVDTLPLL AFMQLFDAFN ASTAGCLRGQ | 540 |
| GRQKIGGYIN LVAFYCLGVP MAYVLAFLYH LGVGGLWLGI TSALVMMSVC QGYAVFHGDR | 600 |
| RRILGAARKR NAETHTS | 617 |

SEQ ID NO: 161
S. cerevisiae

| | |
|---|---|
| atgtctaaac aatttagtca taccaccaac gacagaagat catcgattat ctactccacc | 60 |
| agtgtcggaa aggcagggct tttcacgcct gcagactaca tcccacagga gtcagaagaa | 120 |
| aacttaattg agggcgaaga gcaagagggt agcgaagaag aaccttccta taccggcaat | 180 |
| gacgatgaga cggagaggga aggtgaatac cattcgttat tagatgccaa caattcgcgg | 240 |
| acattgcaac aagaagcgtg gcaacaaggt tatgactctc acgaccgtaa gcgtttgctt | 300 |
| gacgaagaac gggacctgct aatagacaac a | |

1. A recombinant host cell capable of producing one or more steviol glycosides or a steviol glycoside composition in a cell culture, comprising:
(a) a recombinant gene encoding a polypeptide capable of debranching glycogen; and/or
(b) a recombinant gene encoding a polypeptide capable of synthesizing glucose-1-phosphate.

2. The recombinant host cell of claim 1, wherein the polypeptide capable of debranching glycogen is capable of 4-α-glucanotransferase activity and α-1,6-amyloglucosidase activity.

3. The recombinant host cell of claim 1, further comprising:
(c) a gene encoding a polypeptide capable of synthesizing uridine 5′-triphosphate (UTP) from uridine diphosphate (UDP);
wherein the polypeptide capable of synthesizing UTP from UDP comprises a polypeptide having at least 60% sequence identity to the amino acid sequence set forth in SEQ ID NO:123;
(d) a gene encoding a polypeptide capable of converting glucose-6-phosphate to glucose-1-phosphate;
wherein the polypeptide capable of converting glucose-6-phosphate to glucose-1-phosphate comprises a polypeptide having at least 60% sequence identity to the amino acid sequence set forth in any one of SEQ ID NOs:2, 119, or 143 or a polypeptide having at least 55% sequence identity to the amino acid sequence set forth in any one of SEQ ID NOs:141, 145, or 147; and/or
(e) a gene encoding a polypeptide capable of synthesizing uridine diphosphate glucose (UDP-glucose) from UTP and glucose-1-phosphate;
wherein the polypeptide capable of synthesizing UDP-glucose from UTP and glucose-1-phosphate comprises a polypeptide having at least 60% sequence identity to the amino acid sequence set forth in any one of SEQ ID NOs:121 or 127, a polypeptide having at least 55% sequence identity to the amino acid sequence set forth in any one of SEQ ID NOs:125, 129, 133, 135, 137, or 139 or a polypeptide having at least 70% sequence identity to the amino acid sequence set forth in SEQ ID NO:131.

4. The recombinant host cell of claim 1, wherein:
(a) the polypeptide capable of debranching glycogen comprises a polypeptide having at least 60% sequence identity to the amino acid sequence set forth in SEQ ID NO:157; and/or
(b) the polypeptide capable of synthesizing glucose-1-phosphate comprises a polypeptide having at least 55% sequence identity to the amino acid sequence set forth in SEQ ID NO:159.

5. The recombinant host cell of claim 1, further comprising:
(a) a gene encoding a polypeptide capable of glycosylating the steviol or the steviol glycoside at its C-13 hydroxyl group thereof;
wherein the polypeptide capable of glycosylating the steviol or the steviol glycoside at its C-13 hydroxyl group thereof comprises a polypeptide having at least 55% sequence identity to the amino acid sequence set forth in SEQ ID NO:7;
(b) a gene encoding a polypeptide capable of beta 1,3 glycosylation of the C3′ of the 13-O-glucose, 19-O-glucose, or both 13-O-glucose and 19-O-glucose of the steviol glycoside;
wherein the polypeptide capable of beta 1,3 glycosylation of the C3′ of the 13-O-glucose, 19-O-glucose, or both 13-O-glucose and 19-O-glucose of the steviol glycoside comprises a polypeptide having at least 50% sequence identity to the amino acid sequence set forth in SEQ ID NO:9;
(c) a gene encoding a polypeptide capable of glycosylating the steviol or the steviol glycoside at its C-19 carboxyl group thereof;
wherein the polypeptide capable of glycosylating the steviol or the steviol glycoside at its C-19 carboxyl group thereof comprises a polypeptide having at least 55% sequence identity to the amino acid sequence set forth in SEQ ID NO:4;
(d) a gene encoding a polypeptide capable of beta 1,2 glycosylation of the C2′ of the 13-O-glucose, 19-O-glucose, or both 13-O-glucose and 19-O-glucose of the steviol glycoside;
wherein the polypeptide capable of beta 1,2 glycosylation of the C2′ of the 13-O-glucose, 19-O-glucose, or both 13-O-glucose and 19-O-glucose of the steviol glycoside comprises a polypeptide having at least 80% sequence identity to the amino acid sequence set forth in SEQ ID NO:11; a polypeptide having at least 80% sequence identity to the amino acid sequence set forth in SEQ ID NO:13; or a polypeptide having at least 65% sequence identity to the amino acid sequence set forth in SEQ ID NO:16;
(e) a gene encoding a polypeptide capable of synthesizing geranylgeranyl pyrophosphate (GGPP) from farnesyl diphosphate (FPP) and isopentenyl diphosphate (IPP);
wherein the polypeptide capable of synthesizing GGPP comprises a polypeptide having at least 70% sequence identity to the amino acid sequence set forth in any one of SEQ ID NOs:20, 22, 24, 26, 28, 30, 32, or 116:
(f) a gene encoding a polypeptide capable of synthesizing ent-copalyl diphosphate from GGPP;
wherein the polypeptide capable of synthesizing ent-copalyl diphosphate comprises a polypeptide having at least 70% sequence identity to the amino acid sequence set forth in any one of SEQ ID NOs:34, 36, 38, 40, 42, or 120;
(g) a gene encoding an a polypeptide capable of synthesizing ent-kaurene from ent-copalyl diphosphate;
wherein the polypeptide capable of synthesizing ent-kaurene comprises a polypeptide having at least 70% sequence identity to the amino acid sequence set forth in any one of SEQ ID NOs:44, 46, 48, 50, or 52;
(h) a gene encoding a polypeptide capable of synthesizing ent-kaurenoic acid from ent-kaurene;
wherein the polypeptide capable of synthesizing ent-kaurenoic acid comprises a polypeptide having at least 70% sequence identity to the amino acid sequence set forth in any one of SEQ ID NOs:60, 62, 66, 68, 70, 72, 74, 76, or 117;
(i) a gene encoding a polypeptide capable of reducing cytochrome P450 complex;
wherein the polypeptide capable of reducing cytochrome P450 complex comprises a polypeptide having at least 70% sequence identity to the amino acid sequence set forth in any one of SEQ ID NOs:78, 80, 82, 84, 86, 88, 90, 92; and/or
(j) a gene encoding a polypeptide capable of synthesizing steviol from ent-kaurenoic acid;
wherein the polypeptide capable of synthesizing steviol comprises a polypeptide having at least 70% sequence identity to the amino acid sequence set forth in any one of SEQ ID NOs:94, 97, 100, 101, 102, 103, 104, 106, 108, 110, 112, or 114;
wherein at least one of the genes is a recombinant gene.

6. (canceled)

7. The recombinant host cell of claim 1, comprising:
(a) the gene encoding the polypeptide capable of debranching glycogen having at least 60% sequence identity to the amino acid sequence set forth in SEQ ID NO:157;
(b) the gene encoding the polypeptide capable of synthesizing glucose-1-phosphate having at least 55% sequence identity to the amino acid sequence set forth in SEQ ID NO:159;
(c) the gene encoding the polypeptide capable of synthesizing uridine 5′-triphosphate (UTP) from uridine diphosphate (UDP) having at least 60% sequence identity to the amino acid sequence set forth in SEQ ID NO:123;
(d) the gene encoding the polypeptide capable of converting glucose-6-phosphate to glucose-1-phosphate having at least 60% sequence identity to the amino acid sequences set forth in SEQ ID NO:2 or 119; and
(e) the gene encoding the polypeptide capable of synthesizing UDP-glucose from UTP and glucose-1-phosphate having at least 60% sequence identity to the amino acid sequence set forth in SEQ ID NO:121; and
further comprising:
(f) the gene encoding the polypeptide capable of glycosylating the steviol or the steviol glycoside at its C-13 hydroxyl group thereof having at least 55% sequence identity to the amino acid sequence set forth in SEQ ID NO:7;
(g) the gene encoding the polypeptide capable of beta 1,3 glycosylation of the C3′ of the 13-O-glucose, 19-O-glucose, or both 13-O-glucose and 19-O-glucose of the steviol glycoside having at least 50% sequence identity to the amino acid sequence set forth in SEQ ID NO:9;
(h) the gene encoding the polypeptide capable of glycosylating the steviol or the steviol glycoside at its C-19 carboxyl group thereof having at least 55% sequence identity to the amino acid sequence set forth in SEQ ID NO:4; and/or
(i) the gene encoding the polypeptide capable of beta 1,2 glycosylation of the C2′ of the 13-O-glucose, 19-O-glucose, or both 13-O-glucose and 19-O-glucose of the steviol glycoside comprises the polypeptide having at least 80% sequence identity to the amino acid sequence set forth in SEQ ID NO:11; the polypeptide having at least 80% sequence identity to the amino acid sequence set forth in SEQ ID NO:13; or the polypeptide having at least 65% sequence identity to the amino acid sequence set forth in SEQ ID NO:16;
wherein at least one of the genes is a recombinant gene.

8. The recombinant host cell of claim 1, comprising:
(a) the recombinant gene encoding the polypeptide capable of debranching glycogen having at least 60% sequence identity to the amino acid sequence set forth in SEQ ID NO:157; and/or
(b) the recombinant gene encoding the polypeptide capable of synthesizing glucose-1-phosphate having at least 55% sequence identity to the amino acid sequence set forth in SEQ ID NO:159;
wherein the recombinant gene encoding the polypeptide capable of debranching glycogen and/or the recombinant gene encoding the polypeptide capable of synthesizing glucose-1-phosphate are overexpressed relative to a corresponding host cell lacking the one or more recombinant genes.

9. The recombinant host cell of claim 8, wherein:
(a) the gene encoding the polypeptide capable of debranching glycogen and/or the gene encoding the polypeptide capable of synthesizing glucose-1-phosphate are overexpressed by at least 10% relative to a corresponding host cell lacking the one or more recombinant genes;
(b) expression of the one or more recombinant genes increases the amount of UDP-glucose accumulated by the cell by at least 10% relative to a corresponding host lacking the one or more recombinant genes;
(c) expression of the one or more recombinant genes increases the amount of the one or more steviol glycosides produced by the cell by at least 5% relative to a corresponding host cell lacking the one or more recombinant genes;
(d) expression of the one or more recombinant genes increases an amount of RebA, RebD, and/or RebM produced by the cell by at least 5% relative to a corresponding host cell lacking the one or more recombinant genes; and/or
(e) expression of the one or more recombinant genes increases the amount of total steviol glycosides produced by the cell by at least 5% relative to a corresponding host lacking the one or more recombinant genes.

10-14. (canceled)

15. The recombinant host cell of claim 1, wherein:
(a) expression of the one or more recombinant genes decreases the amount of the one of one or more steviol glycosides accumulated by the cell by at least 5% relative to a corresponding host lacking the one or more recombinant genes;
(b) expression of the one or more recombinant genes decreases an amount of 13-SMG accumulated by the cell relative to a corresponding host lacking the one or more recombinant genes; and/or
(c) expression of the one or more recombinant genes decreases the amount of total steviol glycosides produced by the cell by less than 2.5% relative to a corresponding host lacking the one or more recombinant genes.

16-19. (canceled)

20. The recombinant host cell of claim 1, wherein the one or more steviol glycosides is, or the steviol glycoside composition comprises, steviol-13-O-glucoside (13-SMG), steviol-1,2-Bioside, steviol-1,3-Bioside, steviol-19-O-glucoside (19-SMG), 1,2-Stevioside, 1,3-stevioside (RebG), rubusoside, rebaudioside A (RebA), rebaudioside B (RebB), rebaudioside C (RebC), rebaudioside D (RebD), rebaudioside E (RebE), rebaudioside F (RebF), rebaudioside M (RebM), rebaudioside Q (RebQ), rebaudioside I (RebI), dulcoside A, and/or an isomer thereof.

21. The recombinant host cell of claim 1, wherein the recombinant host cell is a plant cell, an insect cell, a fungal cell from Aspergillus genus or a yeast cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species, an algal cell or a bacterial cell from Escherichia coli species or Bacillus genus.

22-23. (canceled)

24. A method of producing one or more steviol glycosides or a steviol glycoside composition in a cell culture, comprising culturing the recombinant host cell of claim 1 in the cell culture, under conditions in which the genes are expressed, and wherein the one or more steviol glycosides or the steviol glycoside composition is produced by the recombinant host cell.

25. The method of claim 24, wherein the genes are constitutively expressed.

26. The method of claim 24, wherein the expression of the genes is induced.

27. The method of claim 24, wherein:
(a) the amount of RebA, RebD, and/or RebM produced by the cell is increased by at least 5% relative to a corresponding host lacking the one or more recombinant genes;
(b) the amount of total steviol glycosides produced by the cell is increased by at least 5% relative to a corresponding host lacking the one or more recombinant genes; and/or
(c) the amount of UDP-glucose accumulated by the cell increases by at least 10% relative to a corresponding host lacking the one or more recombinant genes.

28. The method of claim 24, wherein:
(a) the amount of 13-SMG accumulated by the cell is decreased by at least 10% relative to a corresponding host lacking the one or more recombinant genes; and/or
(b) the amount of total steviol glycosides produced by the cell is decreased by less than 2.5% relative to a corresponding host lacking the one or more recombinant genes.

29-32. (canceled)

33. The method of claim 24, further comprising isolating the produced one or more steviol glycosides or the steviol glycoside composition from the cell culture; wherein the isolating step comprises separating a liquid phase of the cell culture from a solid phase of the cell culture to obtain a supernatant comprising the produced one or more steviol glycosides or the steviol glycoside composition, and:
(a) contacting the supernatant with one or more adsorbent resins in order to obtain at least a portion of the produced one or more steviol glycosides or the steviol glycoside composition; or
(b) contacting the supernatant with one or more ion exchange or reversed-phase chromatography columns in order to obtain at least a portion of the produced one or more steviol glycosides or the steviol glycoside composition; or
(c) crystallizing or extracting the produced one or more steviol glycosides or the steviol glycoside composition;
thereby isolating the produced one or more steviol glycosides or the steviol glycoside composition.

34. (canceled)

35. The method of claim 24, further comprising recovering the one or more steviol glycosides or the steviol glycoside composition from the cell culture; wherein the recovered one or more steviol glycosides or the steviol glycoside composition is enriched for the one or more steviol glycosides relative to a steviol glycoside composition of Stevia plant and has a reduced level of Stevia plant-derived components relative to a steviol glycoside composition obtained from a plant-derived Stevia extract.

36. (canceled)

37. A method for producing one or more steviol glycosides or a steviol glycoside composition, comprising whole-cell bioconversion of a plant-derived or synthetic steviol and/or steviol glycosides in a cell culture of a recombinant host cell using:
(a) a polypeptide capable of debranching glycogen, comprising a polypeptide having at least 60% sequence identity to the amino acid sequence set forth in SEQ ID NO:157; and/or
(b) a polypeptide capable of synthesizing glucose-1-phosphate, comprising a polypeptide having at least 55% sequence identity to the amino acid sequence set forth in SEQ ID NO:159; and further using
(c) a polypeptide capable of synthesizing UTP from UDP, comprising a polypeptide having at least 60% sequence identity to the amino acid sequence set forth in SEQ ID NO:123;
(d) a polypeptide capable of converting glucose-6-phosphate to glucose-1-phosphate, comprising a polypeptide having at least 60% sequence identity to the amino acid sequence set forth in any one of SEQ ID NO:2, 119, or 143; or at least 55% sequence identity to the amino acid sequence set forth in any one of SEQ ID NOs:141, 145, or 147; and/or
(e) a polypeptide capable of synthesizing UDP-glucose from UTP and glucose-1-phosphate, comprising a polypeptide having at least 60% sequence identity to the amino acid sequence set forth in SEQ ID NO:121 or 127; at least 55% sequence identity to the amino acid sequence set forth in any one of SEQ ID NOs:125, 129, 133, 135, 137, or 139; or at least 70% sequence identity to the amino acid sequence set forth in SEQ ID NO:131, and further using:
(f) a polypeptide capable of glycosylating a steviol or a steviol glycoside at its C-13 hydroxyl group thereof;
wherein the polypeptide capable of glycosylating the steviol or the steviol glycoside at its C-13 hydroxyl group thereof comprises a polypeptide having at least 55% sequence identity to the amino acid sequence set forth in SEQ ID NO:7;
(g) a polypeptide capable of beta 1,3 glycosylation of the C3′ of the 13-O-glucose, 19-O-glucose, or both 13-O-glucose and 19-O-glucose of the steviol glycoside;
wherein the polypeptide capable of beta 1,3 glycosylation of the C3′ of the 13-O-glucose, 19-O-glucose, or both 13-O-glucose and 19-O-glucose of the steviol glycoside comprises a polypeptide having at least 50% sequence identity to the amino acid sequence set forth in SEQ ID NO:9;
(h) a polypeptide capable of glycosylating the steviol or the steviol glycoside at its C-19 carboxyl group thereof;
wherein the polypeptide capable of glycosylating the steviol or the steviol glycoside at its C-19 carboxyl group thereof comprises a polypeptide having at least 55% sequence identity to the amino acid sequence set forth in SEQ ID NO:4;
and/or
(i) a polypeptide capable of beta 1,2 glycosylation of the C2′ of the 13-O-glucose, 19-O-glucose, or both 13-O-glucose and 19-O-glucose of a steviol glycoside;
wherein the polypeptide capable of beta 1,2 glycosylation of the C2′ of the 13-O-glucose, 19-O-glucose, or both 13-O-glucose and 19-O-glucose of the steviol glycoside comprises a polypeptide having at least 80% sequence identity to the amino acid sequence set forth in SEQ ID NO:11; a polypeptide having at least 80% sequence identity to the amino acid sequence set forth in SEQ ID NO:13; or a polypeptide having at least 65% sequence identity to the amino acid sequence set forth in SEQ ID NO:16;
wherein at least one of the polypeptides is a recombinant polypeptide expressed in the recombinant host cell; and producing the one or more steviol glycosides or the steviol glycoside composition thereby.

38. (canceled)

39. The method of claim 37, wherein the host cell is a plant cell, a mammalian cell, an insect cell, a fungal cell from Aspergillus genus or a yeast cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species, an algal cell or a bacterial cell from Escherichia coli species or Bacillus genus.

40-41. (canceled)

42. The method of claim 37, wherein the one or more steviol glycosides is, or the steviol glycoside composition comprises, steviol-13-O-glucoside (13-SMG), steviol-1,2-Bioside, steviol-1,3-Bioside, steviol-19-O-glucoside (19-SMG), 1,2-stevioside, 1,3-stevioside (RebG), rubusoside, rebaudioside A (RebA), rebaudioside B (RebB), rebaudioside C (RebC), rebaudioside D (RebD), rebaudioside E (RebE), rebaudioside F (RebF), rebaudioside M (RebM), rebaudioside Q (RebQ), rebaudioside I (RebI), dulcoside A, and/or an isomer thereof.

43. A cell culture, comprising the recombinant host cell of claim 1, the cell culture further comprising:
(a) the one or more steviol glycosides or the steviol glycoside composition produced by the recombinant host cell;
(b) glucose, fructose, sucrose, xylose, rhamnose, UDP-glucose, UDP-rhamnose, UDP-xylose, and/or N-acetyl-glucosamine; and
(c) supplemental nutrients comprising trace metals, vitamins, salts, YNB, and/or amino acids;
wherein the one or more steviol glycosides or the steviol glycoside composition is present at a concentration of at least 1 mg/liter of the cell culture;
wherein UDP-glucose is present in the cell culture at a concentration of at least 100 μM; and
wherein the cell culture is enriched for the one or more steviol glycosides or the steviol glycoside composition relative to a steviol glycoside composition from a Stevia plant and has a reduced level of Stevia plant-derived components relative to a plant-derived Stevia extract.

44. (canceled)

45. A cell lysate from the recombinant host cell of claim 1 grown in the cell culture, comprising:
(a) the one or more steviol glycosides or the steviol glycoside composition produced by the recombinant host cell;
(b) glucose, fructose, sucrose, xylose, rhamnose, UDP-glucose, UDP-rhamnose, UDP-xylose, and/or N-acetyl-glucosamine; and/or
(c) supplemental nutrients comprising trace metals, vitamins, salts, yeast nitrogen base, YNB, and/or amino acids;
wherein the one or more steviol glycosides or the steviol glycoside composition produced by the recombinant host cell is present at a concentration of at least 1 mg/liter of the cell culture.

46. One or more steviol glycosides produced by the recombinant host cell of claim 1;
wherein the one or more steviol glycosides produced by the recombinant host cell are present in relative amounts that are different from a steviol glycoside composition from a Stevia plant and have a reduced level of Stevia plant-derived components relative to a plant-derived Stevia extract.

47. One or more steviol glycosides produced by the method of claim 37;
wherein the one or more steviol glycosides produced are present in relative amounts that are different from a steviol glycoside composition from a Stevia plant and have a reduced level of Stevia plant-derived components relative to a plant-derived Stevia extract.

48. A sweetener composition, comprising the one or more steviol glycosides of claim 46.

49. A food product, a beverage, or a beverage concentrate comprising, the sweetener composition of claim 48.

50. (canceled)
